(19) United States
(12) Patent Application Publication
(10) Pub. No.: US 2025/0163022 A1
(43) Pub. Date: May 22, 2025

(54) PARP1 INHIBITORS

(71) Applicant: Synnovation Therapeutics, Inc., Wilmington, DE (US)

(72) Inventors: Jun Pan, Wilmington, DE (US); Yu Bai, Wilmington, DE (US); Hewen Zheng, Wilmington, DE (US); Yu Li, Wilmington, DE (US); Jeffrey Yang, Wilmington, DE (US); Liangxing Wu, Wilmington, DE (US); Wenqing Yao, Wilmington, DE (US)

(21) Appl. No.: 18/728,407

(22) PCT Filed: Dec. 21, 2022
(86) PCT No.: PCT/US2022/053591

§ 371 Date: Jul. 11, 2024

Related U.S. Application Data
(60) Provisional application No. 63/403,459, filed on Sep. 2, 2022.
(60) Provisional application No. 63/327,228, filed on Apr. 4, 2022.
(60) Provisional application No. 63/292,903, filed on Dec. 22, 2021.

(51) Int. Cl. C07D 401/12; A61K 31/496; A61K 31/517; A61K 31/519; C07D 471/04; C07D 487/06; C07D 491/06; C07D 495/04

(52) U.S. Cl. CPC C07D 401/12; A61K 31/496; A61K 31/517; A61K 31/519; C07D 471/04; C07D 487/06; C07D 491/06; C07D 495/04

(57) ABSTRACT

The present disclosure provides compounds, compositions, and methods useful for inhibiting PARP1, and/or treating a disease, disorder, or condition associated with PARP1, and/or treating cancer.

### TECHNICAL FIELD

The present disclosure provides heterocyclic compounds as well as their pharmaceutical compositions that modulate the activity of PARP1 and are useful in the treatment of various diseases related to PARP1, including cancer.

### BACKGROUND

Poly ADP-Ribose Polymerases (PARPs) are a superfamily of enzymes that comprise at least 17 family members. Some of these PARP enzymes, including PARP1, PARP2, PARP5A, and PARP5B, catalyze NAD+ substrate to covalently attach poly ADP-ribose (PAR), a linear or branched, heterogeneous polymer to acceptor proteins, while other members attach mono ADP-ribose (MAR) to acceptor proteins. Accumulating evidence suggests that PARP enzymes have distinct functions. Among those identified PARPs, PARP1, PARP2 and PARP3 are DNA-dependent of which enzymatic activity is strongly stimulated by endogenous and exogenous DNA damage (van Beek, L. et al. Int. J. Mol. Sci., 2021, 22, 5112). These first three PARP enzyme members are therefore important for the regulation of DNA damage repair through a mechanism called Poly ADP-ribosylation (PARylation).

PARylation is a dynamic, short-lived post-translational modification, which can take place in very few minutes. The polymer generated by PARylation can then be degraded through another enzyme called poly ADP-ribose glycohydrolase (PARG). These key enzymes protect cells from DNA damage-induced cell dysfunction and cell death. Because of the large size and highly charged property of PAR, PARylation dramatically alters the regulation of DNA damage response (DDR), cellular stress response and RNA transcription/processing in various biological systems (Feng, X. et al. Int. Rev. Cell Mol. Biol., 2013, 304, 227; Kraus, W. L., Mol. Cell, 2015, 58, 902; Cohen, M. S., et al. Nat. Chem. Biol., 2018, 14, 236). PARP1, the founding member of the PARP superfamily, contributing to over 90% of PARylation, has been extensively studied for its pivotal role in DNA damage response, especially for the repair of DNA single strand breaks (SSBs) (Durkacz, B. W., et al. Nature, 1980, 283, 593). The basal level of PARylation in quiescent cells is typically below detection. When exposed to genotoxic stress, PARP1 is rapidly activated by self-modification (auto-PARylation), which initiates the DNA damage-response signaling pathways. This process includes a complex cascade of signaling events starting from binding of PARP proteins to the damage sites, to PARylating and recruiting of repair factors, and eventually dissociating from the damage sites (Bai, P., Mol. Cell, 2015, 58, 947). PARP2 is involved in DNA damage repair as well. However, distinct from PARP1, mounting evidence suggests that PARP2 also plays crucial roles in the development and maintenance of hematopoietic cells and some other tissues.

Clinical data have clearly demonstrated the effectiveness of PARP inhibitors in treating a variety of human cancers, particularly the BRCA1/2-mutated, homologous recombination deficient (HRD) cancers. PARP inhibition compromises repair of SSBs by blocking PARylation. On the other hand, PARP inhibitors also trap the PARP protein onto DNA damage sites. PARP trapping leads to blockade of DNA replication, resulting in single-ended DNA double strand breaks (DSBs) due to collapse of replication forks. These breaks require homologous recombination (HR) for faithful repair. Otherwise, these cells would die due to accumulated DNA damage and genomic instability. PARP hyperactivation is frequently observed in cancer patients with HRD tumors. This correlation clearly indicates a hyper-reliance of these tumors on PARP mediated DNA repair pathways (Helleday, T., Mol. Oncol., 2011, 5, 387). These mechanistic studies provide the rationale for targeting HRD cancers with PARP inhibitors.

Although PARP1 is the primary target for developing PARP inhibitors, most if not all current PARP inhibitors also suppress enzymatic activities of other PARPs, particularly PARP2, a close paralog of PARP1 that sharing a 69% identity of its catalytic domain. PARP2 catalyzes only about 10% of cellular PARylation in the presence of PARP1 (Ame, J. C., et al. Bioessays, 2004, 26, 882; Ame, J. C., et al. J. Biol. Chem., 1999, 274, 17860). Despite the functional redundancy with PARP1, PARP2 also has its own unique functions in controlling hematopoiesis, spermatogenesis, adipogenesis and transcriptional regulation. Therefore, pharmacologic inhibition of the PARP2 enzyme may lead to unfavorable effects in aforementioned tissues, consequently resulting in adverse effects in clinical applications (Farres, J., et al. Blood, 2013, 122, 44; Chen, Q., et al. Nat. Commun., 2018, 9, 3233; Gui, B., et al. PNAS, 2019, 116, 14573). Taken together, selective inhibition of PARP1 while retaining the essential functions of PARP2 and other PARP family members is expected to maximize efficacy of PARP inhibitors in treating human cancers while minimizing its unfavorable side effects.

### SUMMARY

The present disclosure provides compounds and/or compositions useful for inhibiting PARP1. In some embodiments, provided compounds and/or compositions are useful for, among other things, treating and/or preventing diseases, disorders, or conditions associated with PARP1.

In some embodiments, the present disclosure provides certain compounds and/or compositions that are useful in medicine, and particularly for treating cancer.

In some embodiments, the present disclosure provides a compound of Formula I:

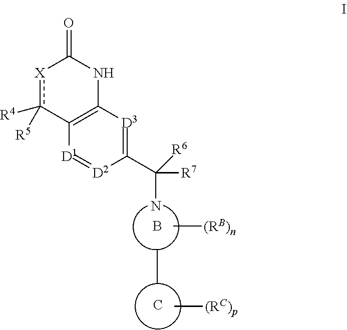

or a pharmaceutically acceptable salt thereof, wherein each of X, R⁴, R⁵, R⁶, R⁷, D¹, D², D³, Ring B, Ring C, R^{B}, R^{C}, n and p is as defined herein.

In some embodiments, provided compounds have structures of any of Formulae II, II-a, II-a-i, III, IV, V, VI, VI-a, VI-b, VII, VIII, VIII-a, VIII-b, VIII-c, VIII-d, IX, IX-a, IX-b, IX-c, and IX-d as described herein.

In some embodiments, the present disclosure provides compositions that comprise and/or deliver a provided compound. In some embodiments, such compositions are pharmaceutical compositions comprising a pharmaceutically acceptable carrier.

The present disclosure further provides methods of inhibiting PARP1 activity, comprising contacting the PARP1 with a compound described herein, or a pharmaceutically acceptable salt thereof.

The present disclosure further provides methods of treating a disease or a disorder associated with PARP1 in a patient by administering to the patient a therapeutically effective amount of a compound of the disclosure, or a pharmaceutically acceptable salt thereof.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

### DETAILED DESCRIPTION

### Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomic, and geometric (or conformational) mixtures of provided compounds are within the scope of the disclosure. For example, in some case, Table 1 shows one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. The isotopically-labeled compounds may have one or more atoms replaced by an atom having an atomic mass or mass number usually found in nature. Examples of isotopes present in compounds of the present disclosure include isotopes of hydrogen, carbon, nitrogen, oxygen, phosphorus, fluorine and chlorine, such as, but not limited to, ²H, ³H, ¹³C, ¹⁴C, ¹⁵N, ¹⁷O, ¹⁸O, ³⁵S and ¹⁸F. Certain isotopically-labeled compounds of the present disclosure, in addition to being useful as therapeutic agents, are also useful in drug and/or substrate tissue distribution assays, as analytical tools or as probes in other biological assays. In one aspect of the present disclosure, tritiated (e.g., ³H) and carbon-14 (e.g., ¹⁴C) isotopes are useful given their ease of detectability. In another aspect of the present invention, replacement of one or more hydrogen atoms with heavier isotopes such as deuterium, (e.g., ²H) can afford certain therapeutic advantages.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocyclylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

Aliphatic: As used herein, the term “aliphatic” refers to a straight-chain (i.e., unbranched) or branched, optionally substituted hydrocarbon chain that is completely saturated or that contains one or more units of unsaturation, or a monocyclic or bicyclic hydrocarbon that is completely saturated or that contains one or more units of unsaturation but which is not aromatic (also referred to herein as “carbocyclic” or “cycloaliphatic”), that, unless otherwise specified, has a single point of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, alkynyl groups and hybrids thereof. In some embodiments, “aliphatic” refers to a straight-chain (i.e., unbranched) or branched, optionally substituted hydrocarbon chain that is completely saturated or that contains one or more units of unsaturation that has a single point of attachment to the rest of the molecule.

Alkyl: The term “alkyl”, used alone or as part of a larger moiety, refers to a saturated, optionally substituted straight or branched hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkenyl: The term “alkenyl”, used alone or as part of a larger moiety, refers to an optionally substituted straight or branched hydrocarbon chain having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Aryl: As used herein, the term “aryl” refers to monocyclic, bicyclic, and polycyclic ring systems having a total of six to fourteen ring members (e.g., C₆₋₁₄), wherein at least one ring in the system is aromatic and wherein each ring in the system contains three to seven ring members. The term “aryl” may be used interchangeably with the term “aryl ring”. In some embodiments, “aryl” refers to an aromatic ring system which includes, but not limited to, phenyl, naphthyl, anthracyl and the like, which may bear one or more substituents. Unless otherwise specified, “aryl” groups are hydrocarbons.

Bivalent: As used herein, the term “bivalent” refers to a chemical moiety with two points of attachment to the rest of the molecule. For example, “bivalent C₁₋₆ aliphatic,” refers to bivalent aliphatic groups that are as defined herein, containing 1-6 aliphatic carbon atoms.

Carbocyclyl: As used herein, the terms “carbocyclyl,” “carbocycle,” and “carbocyclic ring” refer to saturated or partially unsaturated cyclic aliphatic monocyclic, bicyclic, or polycyclic ring systems, as described herein, having from 3 to 14 members, wherein the aliphatic ring system is optionally substituted as described herein. Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₆-C10 bicyclic hydrocarbon that is completely saturated or that contains one or more units of unsaturation, but which is not aromatic, that has a single point of attachment to the rest of the molecule. The term “cycloalkyl” refers to an optionally substituted saturated ring system of about 3 to about 10 ring carbon atoms. In some embodiments, cycloalkyl groups have 3-6 carbons. Exemplary monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Carrier: As used herein, the term “carrier” refers to a diluent, adjuvant, excipient, or vehicle with which a composition is administered. In some embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaryl: As used herein, the terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3 (4H)-one, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: As used herein, the term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, and “heterocyclic ring” are used interchangeably and refer to a stable 3- to 8-membered monocyclic or 6- to 10-membered bicyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)).

Partially Unsaturated: As used herein, the term “partially unsaturated”, when referring to a ring moiety, means a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: As used herein, the term “pharmaceutically acceptable salt” refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

Prevent or prevention: As used herein, the term “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Substituted or optionally substituted: As described herein, compounds of this disclosure may contain “optionally substituted” moieties. In general, the term “substituted,” whether preceded by the term “optionally” or not, means that one or more hydrogens of the designated moiety are replaced with a suitable substituent (i.e., as described below for optionally substituted groups). “Substituted” applies to one or more hydrogens that are either explicit or implicit from the structure (e.g.,

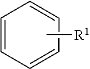

refers to at least

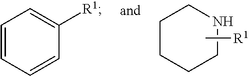

refers to at least

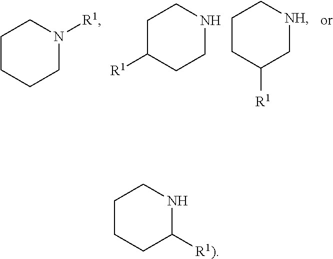

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘}) 2; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)(═NR^{∘})R^{∘}; —(CH₂)₀₋₄S(O)₂OR^{∘}; —(CH₂)₀₋₄OS(O)₂R^{∘}; —(CH₂)₀₋₄—S(O)₂NR^{∘}₂; —(CH₂)₀₋₄S(O)(═NR^{∘})NR^{∘}₂; —(CH₂)₀₋₄S(O)R^{∘}; —N(R^{∘})S(O)₂NR^{∘}₂; —N(R^{∘})S(O)₂R^{∘}; —N(R^{∘})S(O)(═NR^{∘})R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched) alkylene)O—N(R^{∘}2; or —(C₁₋₄ straight or branched) alkylene) C(O)O—N(R^{∘}2, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{∘}, —(CH₂)₀₋₂CH(OR^{•})₂, —O(haloR^{•}), —(CH₂)₀₋₂CN, —N₃, —(CH₂)₀₋₂C(O)R^{•}, —(CH₂)₀₋₂C(O) OH, —(CH₂)₀₋₂C(O)OR^{•}, —(CH₂)₀₋₂C(O)NH₂, —(CH₂)₀₋₂C(O)NHR^{•}, —(CH₂)₀₋₂C(O)NR^{•}₂, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —(CH₂)₀₋₂NHC(O)R^{•}, —(CH₂)₀₋₂NR^{•})C(O)R^{•}, —NO₂, —SiR^{•}₃, —OSiR^{•}₃, —C(O)SR^{•}, —(C₁₋₄ straight or branched alkylene) C(O)OR^{•}, or —SSR^{•} wherein each R^{•}is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†}) S(O)₂R^{†}; wherein each R^{†} is independently hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{∘}is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Treat: As used herein, the term “treat” (also “treatment” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition.

### Provided Compounds

In some embodiments, the present disclosure provides a compound of Formula I:

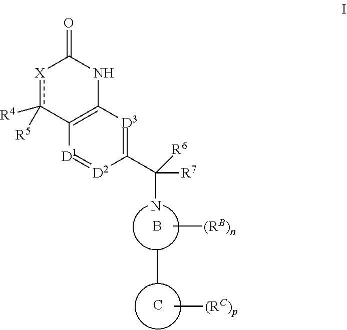

or a pharmaceutically acceptable salt thereof, wherein:
[symbol] is a single or double bond;
X is —C(R¹)═, —C(R¹R²)—, or —N(R^{a})—, as valency allows;
when X is —C(R¹)═, then one of (i)-(iii) applies:
(i)R⁵ is absent;
R¹ and R⁴ are taken together with the carbon atoms to which they are attached to form

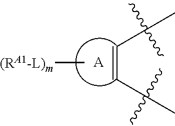

fused to the depicted lactam ring, wherein
Ring A is 5-membered partially unsaturated monocyclic carbocyclyl or 5-membered monocyclic heteroaryl having 1-4 heteroatoms independently selected from nitrogen, oxygen, and sulfur;
(ii)R⁵ is absent;
R⁴ and L^{D1}-R⁸ are taken together with the carbon atoms to which they are attached to form an optionally substituted ring selected from 5- to 7-membered partially unsaturated carbocyclyl or 5- to 7-membered partially unsaturated monocyclic heterocyclyl having 1-3 heteroatoms independently selected from nitrogen, oxygen, and sulfur; or
(iii)R⁵ is absent;
D¹ is S or NR, and D² is absent;
when X is —C(R¹R²)— or —N(R^{a})—:
R¹ and R² are each independently hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or
R¹ and R² are taken together with the carbon atom to which they are attached to form an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur; or
R² and R⁴ are taken together with the carbon atoms to which they are attached to form

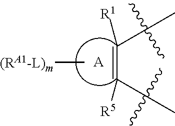

fused to the depicted lactam ring, wherein
Ring A′ is 3- to 7-membered saturated or partially unsaturated monocyclic carbocyclyl or 3- to 7-membered saturated or partially unsaturated monocyclic heterocyclyl having 1-3 heteroatoms independently selected from nitrogen, oxygen, and sulfur;
R^{a} is hydrogen or —L^{R3}-R³,
L^{R3} is a covalent bond or optionally substituted bivalent C₁₋₆ aliphatic;
R³ is hydrogen or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
R⁴ and R⁵ are each independently hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or
R⁴ and R⁵ are taken together with the carbon atom *C to which they are attached to form *C═O, *C═S, *C═NR^{L}, or an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur; or
R⁵ is absent; R⁴ and L^{D1}_R⁸ are taken together with the carbon to which they are attached to form an optionally substituted ring selected from phenyl, 5- to 6-membered monocyclic heteroaryl having 1-4 heteroatoms independently selected from nitrogen, oxygen, and sulfur;
R^{L} is hydrogen, —CN, —OR^{L1}, or optionally substituted C₁₋₆ alkyl;
R^{L1} is hydrogen, C₁₋₆ alkyl, or C₁₋₆ haloalkyl;
each L is independently a covalent bond or optionally substituted bivalent C₁₋₆ aliphatic;
each R^{A1} is independently halogen, —CN, —OR, —SR, —N(R)₂, —N(R)₃, —NO₂, —C(O)R′, —C(O)OR, —C(O)N(R)₂, —OC(O)R′, —OC(O)N(R)₂, —OC(O)OR, —OSO₂R′, —OSO₂R′, N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
R⁶ and R⁷ are each independently hydrogen, halogen, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or
R⁶ and R⁷ are taken together with the carbon to which they are attached to form an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur;
D¹ is C-L^{D1}-R⁸, N, NR, or S;
D² is absent, C-L^{D2}-R⁹, or N, wherein when D¹ is S or NR, D² is absent;
D³ is CR¹⁰ or N;
L^{D1} is a covalent bond or optionally substituted bivalent C₁₋₆ aliphatic;
L^{D2} is a covalent bond or optionally substituted bivalent C₁₋₆ aliphatic;
R⁸ is hydrogen, halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
R⁹ and R¹⁰ are each independently hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur;
Ring B is 3- to 7-membered saturated or partially unsaturated monocyclic heterocyclylene having 1-3 heteroatoms independently selected from nitrogen, oxygen, and sulfur, 6- to 10-membered saturated or partially unsaturated bicyclic heterocyclylene having 1-4 heteroatoms independently selected from nitrogen, oxygen, and sulfur, or 9- to 16-membered saturated or partially unsaturated polycyclic heterocyclylene having 1-5 heteroatoms independently selected from nitrogen, oxygen, and sulfur;
Ring C is phenyl, 8- to 10-membered bicyclic aryl, 10- to 14-membered polycyclic aryl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 10- to 16-membered polycyclic heteroaryl having 1-5 heteroatoms independently selected from nitrogen, oxygen, and sulfur, or 6- to 10-membered saturated or partially unsaturated bicyclic heterocyclyl having 1-4 heteroatoms independently selected from nitrogen, oxygen, and sulfur;
each R^{B} is independently -L^{RB}-R¹¹;
each L^{RB} is independently a covalent bond or optionally substituted bivalent C₁₋₆ aliphatic;
each R^{C} is independently -L^{RC}-R¹²;
each L^{RC} is independently a covalent bond or optionally substituted bivalent C₁₋₆ aliphatic;
R¹¹ and R¹² are each independently halogen, ═O, —CN, —OR, —SR, —N(R)₂, —N′ (R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
a R^{B} and a R^{C} are taken together with their intervening atoms to form Ring D fused with one or both of Ring B and Ring C, wherein
Ring D is an optionally substituted ring selected from 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 6- to 8-membered saturated or partially unsaturated bicyclic heterocyclyl having 1-4 heteroatoms independently selected from nitrogen, oxygen, and sulfur, phenyl, and 5- to 6-membered monocyclic heteroaryl having 1-4 heteroatoms independently selected from nitrogen, oxygen, and sulfur;
each R is independently hydrogen or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
two R when attached to the same nitrogen atom are taken together to form optionally substituted 3- to 7-membered saturated or partially unsaturated monocyclic heterocyclyl having 0-2 additional heteroatoms independently selected from nitrogen, oxygen, and sulfur;
each R′ is independently an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
two R′ when attached to the same nitrogen atom are taken together to form optionally substituted 3- to 7-membered saturated or partially unsaturated monocyclic heterocyclyl having 0-2 additional heteroatoms independently selected from nitrogen, oxygen, and sulfur;
each R^{m} is independently —OH, —CN, or R;
m is 0, 1, 2, 3, or 4;
n is 0, 1, 2, 3, or 4; and
p is 0, 1, 2, 3, 4, or 5.

In some embodiments:
[symbol] is a single or double bond;
X is —C(R¹)—, —C(R¹R²)—, or —N(R^{a})—, as valency allows;
when X is —C(R¹)═, then one of (i)-(iii) applies:
(i)R⁵ is absent;
R¹ and R⁴ are taken together with the carbon atoms to which they are attached to form

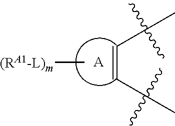

fused to the depicted lactam ring, wherein
Ring A is 5-membered partially unsaturated monocyclic carbocyclyl or 5-membered monocyclic heteroaryl having 1-4 heteroatoms independently selected from nitrogen, oxygen, and sulfur;
(ii)R⁵ is absent;
R⁴ and L^{D1}-R⁸ are taken together with the carbon atoms to which they are attached to form a 5- to 7-membered partially unsaturated carbocyclyl or 5- to 7-membered partially unsaturated monocyclic heterocyclyl having 1-3 heteroatoms independently selected from nitrogen, oxygen, and sulfur, wherein the 5- to 7-membered partially unsaturated carbocyclyl or 5- to 7-membered partially unsaturated monocyclic heterocyclyl are each optionally substituted by 1, 2, 3, or 4 independently selected R^{4A} substituents; or
(iii)R⁵ is absent;
D¹ is S or NR, and D² is absent;
when X is —C(R¹R²)— or —N(R^{a})—;
R¹ and R² are each independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{1A} substituents; or
R¹ and R² are taken together with the carbon atom to which they are attached to form a 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl having 1-3 heteroatoms independently selected from nitrogen, oxygen, and sulfur, or a 6- to 10-membered saturated or partially unsaturated bicyclic heterocyclyl having 1-4 heteroatoms independently selected from nitrogen, oxygen, and sulfur, wherein the 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl, and 6- to 10-membered saturated or partially unsaturated bicyclic heterocyclyl are each optionally substituted with 1, 2, 3, or 4 independently selected R^{1A} substituents; or
R² and R⁴ are taken together with the carbon atoms to which they are attached to form

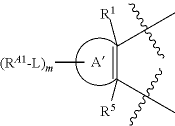

fused to the depicted lactam ring, wherein
Ring A′ is 3- to 7-membered saturated or partially unsaturated monocyclic carbocyclyl or 3- to 7-membered saturated or partially unsaturated monocyclic heterocyclyl having 1-3 heteroatoms independently selected from nitrogen, oxygen, and sulfur;
R^{a} is hydrogen or —L^{R3}-R³,
L^{R3} is a covalent bond or a bivalent C₁₋₆ aliphatic, wherein the bivalent C₁₋₆ aliphatic is optionally substituted with 1, 2, 3, or 4 independently selected RN substituents;
R³ is selected from hydrogen, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{3A} substituents;
R⁴ and R⁵ are each independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{4A} substituents; or
R⁴ and R⁵ are taken together with the carbon atom *C to which they are attached to form *C═O, *C═S, *C═NR^{L}, a 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl having 1-3 heteroatoms independently selected from nitrogen, oxygen, and sulfur, or 6- to 10-membered saturated or partially unsaturated bicyclic heterocyclyl having 1-4 heteroatoms independently selected from nitrogen, oxygen, and sulfur, wherein the 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl, and 6- to 10-membered saturated or partially unsaturated bicyclic heterocyclyl are each optionally substituted with 1, 2, 3, or 4 independently selected R^{4A} substituents; or
R⁵ is absent, and R⁴ and L^{D1}-R⁸, taken together with the carbon to which they are attached, form a group selected from phenyl and 5- to 6-membered monocyclic heteroaryl having 1-4 heteroatoms independently selected from nitrogen, oxygen, and sulfur, wherein the phenyl and 5- to 6-membered monocyclic heteroaryl having 1-4 heteroatoms independently selected from nitrogen, oxygen, and sulfur are each optionally substituted with 1, 2, 3, or 4 independently selected R^{4A} substituents;
R^{L} is hydrogen, —CN, —OR^{L1}, or C₁₋₆ alkyl, wherein the C₁₋₆ alkyl is optionally substituted with 1, 2, 3, or 4 independently selected RN substituents;
R^{L1} is hydrogen, C₁₋₆ alkyl, or C₁₋₆ haloalkyl;
each L is independently a covalent bond or a bivalent C₁₋₆ aliphatic, wherein the bivalent C₁₋₆ aliphatic is optionally substituted with 1, 2, 3, or 4 independently selected R^{N} substituents;
each R^{A1} is independently selected from halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{B1} substituents;
R⁶ and R⁷ are each independently hydrogen, halogen, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{6A} substituents; or
R⁶ and R⁷ are taken together with the carbon to which they are attached to form a 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl having 1-3 heteroatoms independently selected from nitrogen, oxygen, and sulfur, or a 6- to 10-membered saturated or partially unsaturated bicyclic heterocyclyl having 1-4 heteroatoms independently selected from nitrogen, oxygen, and sulfur, wherein the 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl, and 6- to 10-membered saturated or partially unsaturated bicyclic heterocyclyl are each optionally substituted with 1, 2, 3, or 4 independently selected R^{6A} substituents;
D¹ is C-L^{D1}-R⁸, N, NR, or S;
D² is absent, C-L^{D2}-R⁹, or N, wherein when D¹ is S or NR, D² is absent;
D³ is CR¹⁰ or N;
L^{D1} is a covalent bond or a bivalent C₁₋₆ aliphatic, wherein the bivalent C₁₋₆ aliphatic is optionally substituted with 1, 2, 3, or 4 independently selected RN substituents;
L^{D2} is a covalent bond or a bivalent C₁₋₆ aliphatic, wherein the bivalent C₁₋₆ aliphatic is optionally substituted with 1, 2, 3, or 4 independently selected RN substituents;
R⁸ is selected from hydrogen, halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{8A} substituents;
R⁹ and R¹⁰ are each independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{9A} substituents;
Ring B is 3- to 7-membered saturated or partially unsaturated monocyclic heterocyclylene having 1-3 heteroatoms independently selected from nitrogen, oxygen, and sulfur, 6- to 10-membered saturated or partially unsaturated bicyclic heterocyclylene having 1-4 heteroatoms independently selected from nitrogen, oxygen, and sulfur, or 9- to 16-membered saturated or partially unsaturated polycyclic heterocyclylene having 1-5 heteroatoms independently selected from nitrogen, oxygen, and sulfur;
Ring C is phenyl, 8- to 10-membered bicyclic aryl, 10- to 14-membered polycyclic aryl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 10- to 16-membered polycyclic heteroaryl having 1-5 heteroatoms independently selected from nitrogen, oxygen, and sulfur, or 6- to 10-membered saturated or partially unsaturated bicyclic heterocyclyl having 1-4 heteroatoms independently selected from nitrogen, oxygen, and sulfur;
each R^{B} is independently -L^{RB}-R¹¹;
each L^{RB} is independently a covalent bond or a bivalent C₁₋₆ aliphatic, wherein the bivalent C₁₋₆ aliphatic is optionally substituted with 1, 2, 3, or 4 independently selected R^{N} substituents;
each R^{C} is independently -L^{RC}-R 12,
each L^{RC} is independently a covalent bond or a bivalent C₁₋₆ aliphatic, wherein the bivalent C₁₋₆ aliphatic is optionally substituted with 1, 2, 3, or 4 independently selected R^{N} substituents;
R¹¹ and R¹² are each independently selected from halogen, —O, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{11A} substituents; or
a R^{B} and a R^{C} are taken together with their intervening atoms to form Ring D fused with one or both of Ring B and Ring C, wherein
Ring D is selected from 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 6- to 8-membered saturated or partially unsaturated bicyclic heterocyclyl having 1-4 heteroatoms independently selected from nitrogen, oxygen, and sulfur, phenyl, and 5- to 6-membered monocyclic heteroaryl having 1-4 heteroatoms independently selected from nitrogen, oxygen, and sulfur, wherein the 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, 6- to 8-membered saturated or partially unsaturated bicyclic heterocyclyl, phenyl, and 5- to 6-membered monocyclic heteroaryl are each optionally substituted with 1, 2, 3, or 4 independently selected R^{D1} substituents;
each R is independently selected from hydrogen, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected RN substituents; or
two R when attached to the same nitrogen atom are taken together to form a 3- to 7-membered saturated or partially unsaturated monocyclic heterocyclyl having 0-2 additional heteroatoms independently selected from nitrogen, oxygen, and sulfur, wherein the 3- to 7-membered saturated or partially unsaturated monocyclic heterocyclyl is optionally substituted with 1, 2, 3, or 4 independently selected RN substituents;
each R′ is independently selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected RN substituents; or
two R′ when attached to the same nitrogen atom are taken together to a 3- to 7-membered saturated or partially unsaturated monocyclic heterocyclyl having 0-2 additional heteroatoms independently selected from nitrogen, oxygen, and sulfur, wherein the 3- to 7-membered saturated or partially unsaturated monocyclic heterocyclyl is optionally substituted with 1, 2, 3, or 4 independently selected RN substituents;
each R^{1A}, R^{3A}, R^{4A}, R^{6A}, R^{8A}, R^{9A}, R^{11A}, R^{B1}, R^{D1} and R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR∘)NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘})₂, —P(O)₂R^{∘}, —P(O)R^{∘}₂, —OP(O)R^{∘}₂, —OP(O)(OR^{∘}₂, —SiR^{∘}₃, —(C₁₋₄ straight or branched) alkylene)O—N(R^{∘})₂, and —(C₁₋₄ straight or branched) alkylene) C(O)O—N(R^{∘})₂;
each R^{∘} is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur;
or two independent occurrences of R^{∘}, taken together with their intervening atoms, form a 3- to 12-membered saturated, partially unsaturated, or aryl mono- or bicyclic ring having 0-4 heteroatoms independently selected from nitrogen, oxygen, or sulfur
each R^{m} is independently —OH, —CN, or R;
m is 0, 1, 2, 3, or 4;
n is 0, 1, 2, 3, or 4; and
p is 0, 1, 2, 3, 4, or 5.

In some embodiments, the present disclosure provides a compound of Formula II:

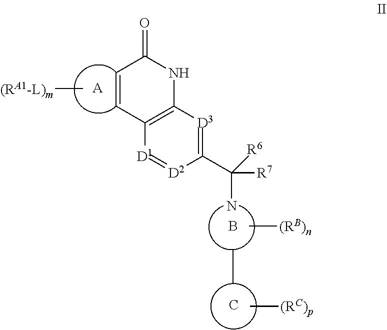

or a pharmaceutically acceptable salt thereof, wherein each of R⁶, R⁷, D¹, D², D³, Ring A, Ring B, Ring C, R^{A1}, R^{B}, R^{C}, L, m, n, and p is as defined above for Formula I and described in classes and subclasses herein, both singly and in combination.

In some embodiments, the present disclosure provides a compound of Formula II-a:

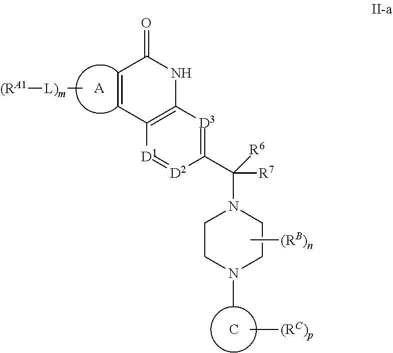

or a pharmaceutically acceptable salt thereof, wherein each of R⁶, R⁷, D¹, D², D³, Ring A, Ring C, R^{A1}, R^{B}, R^{C}, L, m, n, and p is as defined above for Formula I and described in classes and subclasses herein, both singly and in combination.

In some embodiments, the present disclosure provides a compound of Formula II-a-i:

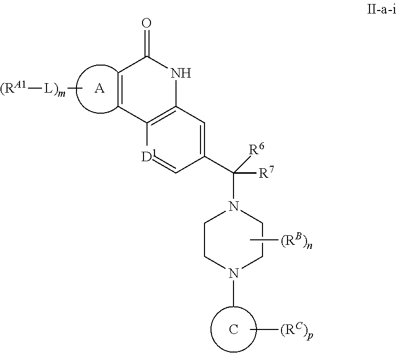

or a pharmaceutically acceptable salt thereof, wherein each of R⁶, R⁷, D¹, Ring A, Ring C, R^{A1}, R^{B}, R^{C}, L, m, n, and p is as defined above for Formula I and described in classes and subclasses herein, both singly and in combination.

In some embodiments, the present disclosure provides a compound of Formula III:

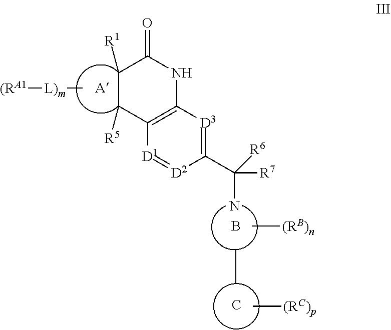

or a pharmaceutically acceptable salt thereof, wherein each of R¹, R⁵, R⁶, R⁷, D¹, D², D³, Ring A′, Ring B, Ring C, R^{A1}, R^{B}, R^{C}, L, m, n, and p is as defined above for Formula I and described in classes and subclasses herein, both singly and in combination.

In some embodiments, the present disclosure provides a compound of Formula IV:

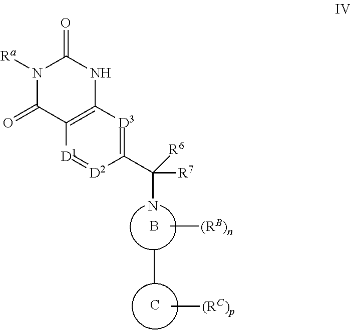

or a pharmaceutically acceptable salt thereof, wherein each of R^{a}, R⁶, R⁷, D¹, D², D³, Ring B, Ring C, R^{B}, R^{C}, n, and p is as defined above for Formula I and described in classes and subclasses herein, both singly and in combination.

In some embodiments, the present disclosure provides a compound of Formula V:

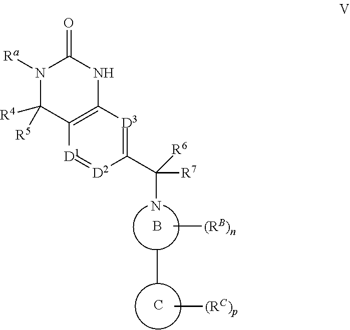

or a pharmaceutically acceptable salt thereof, wherein each of R^{a}, R⁴, R⁵, R⁶, R⁷, D¹, D², D³, Ring B, Ring C, R^{B}, R^{C}, n, and p is as defined above for Formula I and described in classes and subclasses herein, both singly and in combination.

In some embodiments, the present disclosure provides a compound of Formula VI:

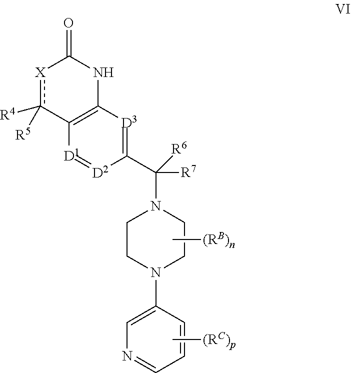

or a pharmaceutically acceptable salt thereof, wherein each of X, R⁴, R⁵, R⁶, R⁷, D¹, D², D³, R^{B}, R^{C}, n, and p is as defined above for Formula I and described in classes and subclasses herein, both singly and in combination.

In some embodiments, the present disclosure provides a compound of Formula VI-a:

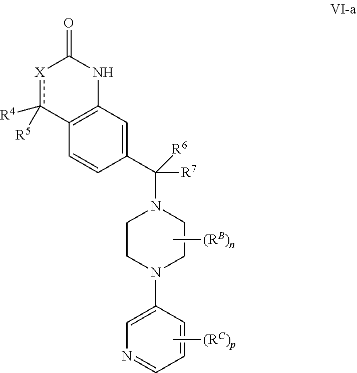

or a pharmaceutically acceptable salt thereof, wherein each of X, R⁴, R⁵, R⁶, R⁷, R^{B}, R^{C}, n, and p is as defined above for Formula I and described in classes and subclasses herein, both singly and in combination.

In some embodiments, the present disclosure provides a compound of Formula VI-b:

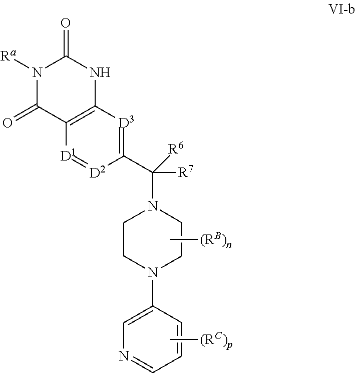

or a pharmaceutically acceptable salt thereof, wherein each of R^{a}, R⁶, R⁷, D¹, D², D³, R^{B}, R^{C}, n, and p is as defined above for Formula I and described in classes and subclasses herein, both singly and in combination.

In some embodiments, the present disclosure provides a compound of Formula VII:

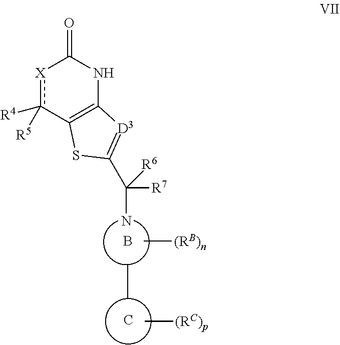

or a pharmaceutically acceptable salt thereof, wherein each of X, R⁴, R⁵, R⁶, R⁷, D³, Ring B, Ring C, R^{B}, R^{C}, n, and p is as defined above for Formula I and described in classes and subclasses herein, both singly and in combination.

In some embodiments, the present disclosure provides a compound of Formula VIII:

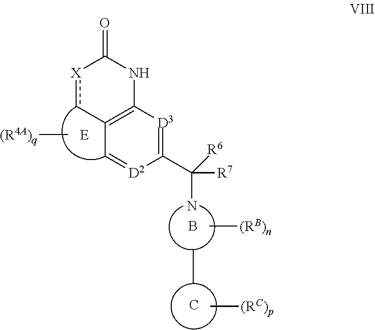

or a pharmaceutically acceptable salt thereof, wherein each of X, D², D³, R^{4A}, R⁶, R⁷, Ring B, Ring C, R^{B}, R^{C}, n, and p are as defined above for Formula I and described in classes and subclasses herein, both singly and in combination;
when X is —C(R¹)—, Ring E is selected from 5- to 7-membered partially unsaturated carbocyclyl and 5- to 7-membered partially unsaturated monocyclic heterocyclyl having 1-3 heteroatoms independently selected from nitrogen, oxygen, and sulfur;

when X is —C(R¹R²)— or —N(R^{a})—, Ring E is selected from phenyl and 5- to 6-membered monocyclic heteroaryl having 1-4 heteroatoms independently selected from nitrogen, oxygen, and sulfur; and
q is 0, 1, 2, 3, or 4.

In some embodiments, the compound provided herein is a compound of Formula VIII-a, VIII-b, VIII-c, or VIII-d:

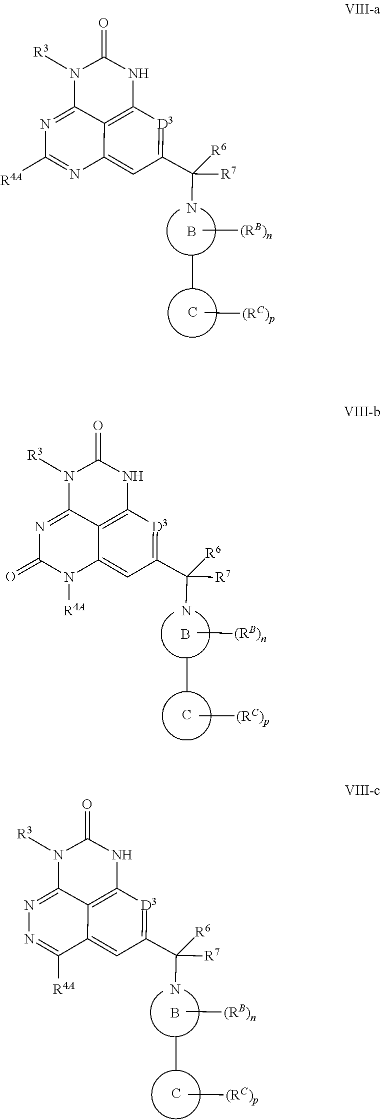

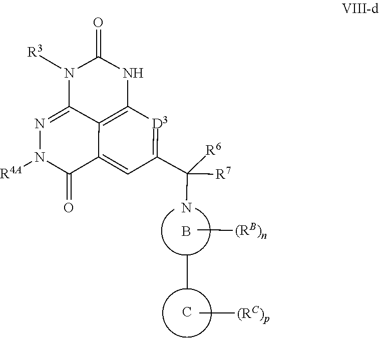

or a pharmaceutically acceptable salt thereof, wherein each of D³, R³, R^{4A}, R⁶, R⁷, Ring B, Ring C, R^{B}, R^{C}, n, and p are as defined above for Formula I and described in classes and subclasses herein, both singly and in combination.

In some embodiments, the present disclosure provides a compound of Formula IX:

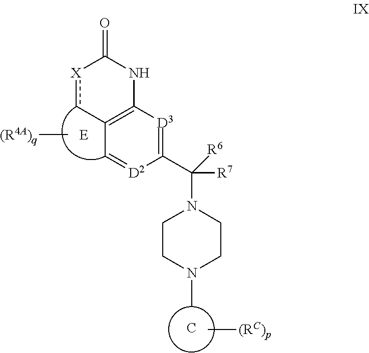

or a pharmaceutically acceptable salt thereof, wherein each of X, D², D³, R^{4A}, R⁶, R⁷, Ring C, R^{C}, and p are as defined above for Formula I and described in classes and subclasses herein, both singly and in combination;
when X is —C(R¹)═, Ring E is selected from 5- to 7-membered partially unsaturated carbocyclyl and 5- to 7-membered partially unsaturated monocyclic heterocyclyl having 1-3 heteroatoms independently selected from nitrogen, oxygen, and sulfur;
when X is —C(R¹R²)— or —N(R^{a})—, Ring E is selected from phenyl and 5- to 6-membered monocyclic heteroaryl having 1-4 heteroatoms independently selected from nitrogen, oxygen, and sulfur; and
q is 0, 1, 2, 3, or 4.

In some embodiments of any of Formulae I, VIII, and IX, Ring E is a pyrimidine, pyrimidinone, pyridazine, or pyridazinone ring.

In some embodiments of any of Formulae I, VIII, and IX, Ring E is a pyrimidine ring.

In some embodiments of any of Formulae I, VIII, and IX, q is 0, 1, or 2.

In some embodiments of any of Formulae I, VIII, and IX, q is 0.

In some embodiments of any of Formulae I, VIII, and IX, q is 1.

In some embodiments of any of Formulae I, VIII, and IX, q is 2.

In some embodiments, the compound provided herein is a compound of Formula IX-a, IX-b, IX-c, or IX-d:

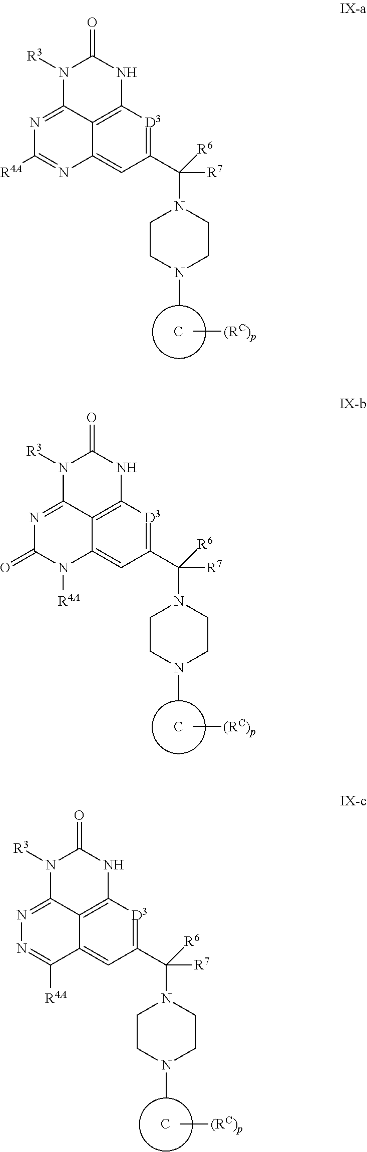

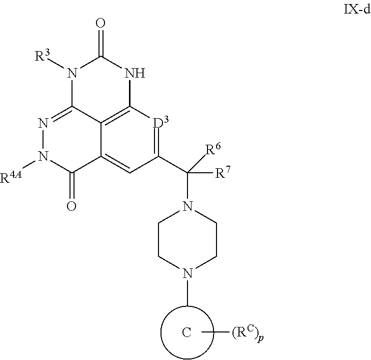

or a pharmaceutically acceptable salt thereof, wherein each of D³, R³, R^{4A}, R⁶, R⁷, Ring C, R^{C}, and p are as defined above for Formula I and described in classes and subclasses herein, both singly and in combination.

As described above, in some embodiments of any of Formulae I, VI, VI-a, and VII [symbol] is a single or double bond. In some embodiments, [symbol] is a single bond. In some embodiments, [symbol] is a double bond.

As described above, in some embodiments of any of Formulae I, VI, VI-a, and VII, X is —C(R¹)═, —C(R¹R²)—, or —N(R^{a})—, as valency allows. In some embodiments, X is —C(R¹)—. In some embodiments, X is —C(R¹R²)—. In some embodiments, X is —N(R^{a})—. In some embodiments of any of Formulae I, VIII, VIII-a, VIII-b, VIII-c, VIII-d, IX, IX-a, IX-b, IX-c, and IX-d, X is —N(R^{a})—.

As described above, in some embodiments of any of Formulae I, VI, VI-a, and VII, when X is —C(R¹)═, R⁵ is absent.

As described above, in some embodiments of any of Formulae I, VI, VI-a, and VII, when X is —C(R¹)—, R¹ and R⁴ are taken together with the carbon atoms to which they are attached to form

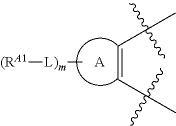

fused to the depicted lactam ring.

As described above, in some embodiments of any of Formulae I, II, II-a, II-a-i, VI, ad VI-a, and VII, Ring A is 5-membered partially unsaturated monocyclic carbocyclyl or 5-membered monocyclic heteroaryl having 1-4 heteroatoms independently selected from nitrogen, oxygen, and sulfur.

In some embodiments, Ring A is 5-membered partially unsaturated monocyclic carbocyclyl. In some embodiments, Ring A is 5-membered monocyclic heteroaryl having 1-4 heteroatoms independently selected from nitrogen, oxygen, and sulfur. In some embodiments, Ring A is 5-membered monocyclic heteroaryl having 1-2 heteroatoms independently selected from nitrogen, oxygen, and sulfur. In some embodiments, Ring A is 5-membered monocyclic heteroaryl having 1-2 nitrogen atoms. In some embodiments, Ring A is 5-membered monocyclic heteroaryl having 1 nitrogen atom. In some embodiments, Ring A is pyrrolyl or pyrazolyl. In some embodiments, Ring A is pyrrolyl. In some embodiments, Ring A is pyrazolyl.

In some embodiments,

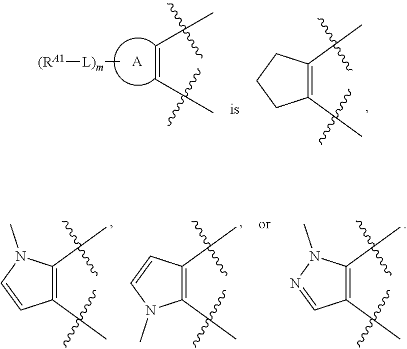

In some embodiments,

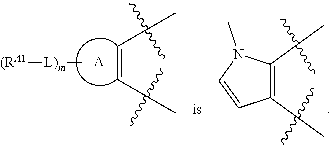

As described above, in some embodiments of any of Formulae I, II, II-a, II-a-i, VI, VI-a, and VII, R⁴ and L^{D1}-R⁸ are taken together with the carbon atoms to which they are attached to form an optionally substituted ring selected from 5- to 7-membered partially unsaturated carbocyclyl or 5- to 7-membered partially unsaturated monocyclic heterocyclyl having 1-3 heteroatoms independently selected from nitrogen, oxygen, and sulfur. In some embodiments, R⁴ and L^{D1}-R⁸ are taken together with the carbon atoms to which they are attached to form an optionally substituted ring selected from 6-membered partially unsaturated monocyclic heterocyclyl having 1-2 heteroatoms independently selected from nitrogen, oxygen, and sulfur. In some embodiments, R⁴ and L^{D1}-R⁸ are taken together with the carbon atoms to which they are attached to form an optionally substituted ring selected from 6-membered partially unsaturated monocyclic heterocyclyl having 1 oxygen heteroatom.

In some embodiments, R⁴ and L^{D1}-R⁸ are taken together with the carbon atoms to which they are attached to form a 5- to 7-membered partially unsaturated carbocyclyl or 5- to 7-membered partially unsaturated monocyclic heterocyclyl having 1-3 heteroatoms independently selected from nitrogen, oxygen, and sulfur, wherein the 5- to 7-membered partially unsaturated carbocyclyl or 5- to 7-membered partially unsaturated monocyclic heterocyclyl are each optionally substituted by 1, 2, 3, or 4 independently selected R^{4A} substituents; and
each R^{4A} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}), —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched alkylene)O—N(R^{∘})₂, and —(C₁₋₄ straight or branched) alkylene) C(O)O—N(R^{∘})₂.

As described above, in some embodiments of any of Formulae I, V, and VI, R⁵ is absent, and R⁴ and L^{D1}-R⁸ are taken together with the carbon atoms to which they are attached to form an optionally substituted ring selected from phenyl, or 5- to 6-membered monocyclic heteroaryl having 1-4 heteroatoms independently selected from nitrogen, oxygen, and sulfur.

In some embodiments, X is NR^{a}, R⁵ is absent, and R⁴ and L^{D1}-R⁸ are taken together with the carbon atoms to which they are attached to form an optionally substituted ring selected from phenyl, or 5- to 6-membered monocyclic heteroaryl having 1-4 heteroatoms independently selected from nitrogen, oxygen, and sulfur.

In some embodiments, R⁵ is absent, R⁴ and L^{D1}-R⁸ are taken together with the carbon atoms to which they are attached to form a ring selected from phenyl, or 5- to 6-membered monocyclic heteroaryl having 1-4 heteroatoms independently selected from nitrogen, oxygen, and sulfur, wherein the phenyl, or 5- to 6-membered monocyclic heteroaryl are each optionally substituted by 1, 2, 3, or 4 independently selected R^{4A} substituents;

In some embodiments, X is NR^{a}, R⁵ is absent, R⁴ and L^{D1}-R⁸ are taken together with the carbon atoms to which they are attached to form a ring selected from phenyl, or 5- to 6-membered monocyclic heteroaryl having 1-4 heteroatoms independently selected from nitrogen, oxygen, and sulfur, wherein the phenyl, or 5- to 6-membered monocyclic heteroaryl are each optionally substituted by 1, 2, 3, or 4 independently selected R^{4A} substituents;

In some embodiments, R⁴ and L^{D1}-R⁸ are taken together with the carbon atoms to which they are attached form a ring selected from

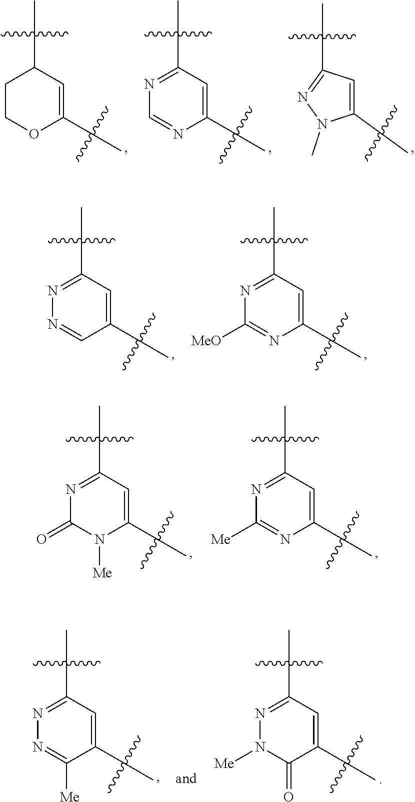

In some embodiments of any of Formulae I, VIII, VIII-a, VIII-b, VIII-c, VIII-d, IX, IX-a, IX-b, IX-c, and IX-d, R⁴ and L^{D1}-R⁸ are taken together with the carbon atoms to which they are attached form a ring selected from

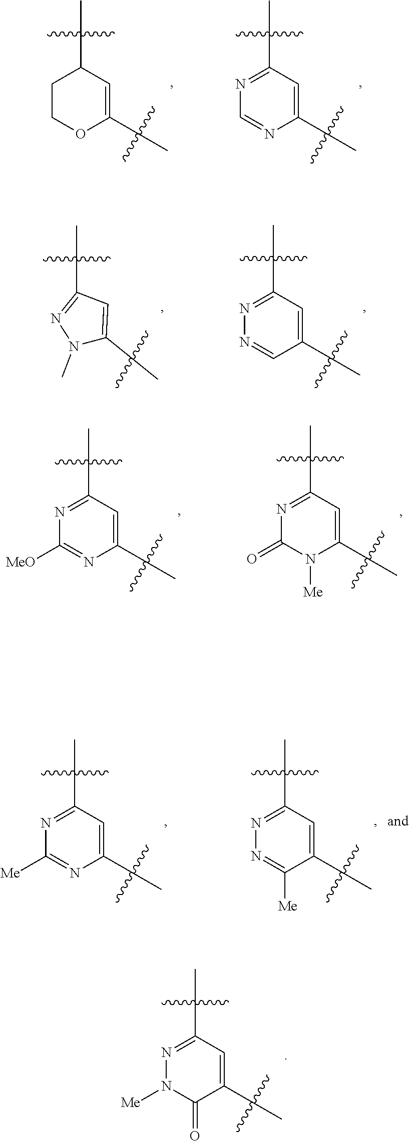

In some embodiments, R⁴ and L^{D1}-R⁸ are taken together with the carbon atoms to which they are attached form a ring selected from

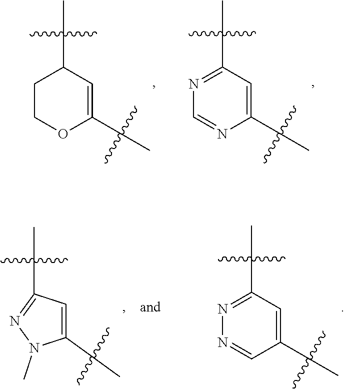

As described above, in some embodiments of any of Formulae I, VI, VI-a, and VII, when X is —C(R¹R²)—, R¹ and R² are each independently hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or R¹ and R² are taken together with the carbon atom to which they are attached to form an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur; or R² and R⁴ are taken together with the carbon atoms to which they are attached to form

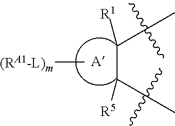

fused to the depicted lactam ring.

In some embodiments of any of Formulae I, VI, VI-a, and VII, when X is-C(R¹R²)—, or in some embodiments of Formula III, R¹ is hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R¹ is selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{1A} substituents; and
each R^{1A} independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘})₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, R¹ is hydrogen.

In some embodiments of any of Formulae I, VI, VI-a, and VII, when X is —C(R¹R²)—, R² is hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R² is selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{1A} substituents; and
each R^{1A} independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR∘)NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘}2, and —(C₁₋₄ straight or branched) alkylene) C(O)O—N(R^{∘}₂.

In some embodiments, R¹ and R² are taken together with the carbon atom to which they are attached to form a 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl having 1-3 heteroatoms independently selected from nitrogen, oxygen, and sulfur, or a 6- to 10-membered saturated or partially unsaturated bicyclic heterocyclyl having 1-4 heteroatoms independently selected from nitrogen, oxygen, and sulfur, wherein the 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl, and 6- to 10-membered saturated or partially unsaturated bicyclic heterocyclyl are each optionally substituted with 1, 2, 3, or 4 independently selected R^{1A} substituents; and
each R^{1A} independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘})₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, R² is hydrogen.

In some embodiments of any of Formulae I, VI, VI-a, and VII, when X is-C(R¹R²)—, R¹ and R² are taken together with the carbon atom to which they are attached to form an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.

In some embodiments, R¹ and R² are taken together with the carbon atom to which they are attached to form optionally substituted 3- to 7-membered saturated or partially unsaturated monocyclic carbocyclyl. In some embodiments, R¹ and R² are taken together with the carbon atom to which they are attached to form optionally substituted 3-membered saturated or partially unsaturated monocyclic carbocyclyl. In some embodiments, R¹ and R² are taken together with the carbon atom to which they are attached to form optionally substituted cyclopropyl.

In some embodiments of any of Formulae I, VI, VI-a, and VII, when X is-C(R¹R²)—, R² and R⁴ are taken together with the carbon atoms to which they are attached to form

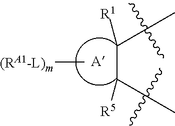

fused to the depicted lactam ring.

As described above, in some embodiments of any of Formulae I, III, VI, and VI-a, Ring A′ is 3- to 7-membered saturated or partially unsaturated monocyclic carbocyclyl or 3- to 7-membered saturated or partially unsaturated monocyclic heterocyclyl having 1-3 heteroatoms independently selected from nitrogen, oxygen, and sulfur.

In some embodiments, Ring A′ is 3- to 7-membered saturated or partially unsaturated monocyclic carbocyclyl. In some embodiments, Ring A′ is 3-membered saturated or partially unsaturated monocyclic carbocyclyl. In some embodiments, Ring A′ is cyclopropyl.

In some embodiments,

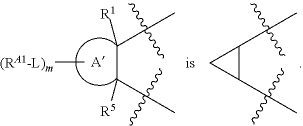

As described above, in some embodiments of any of Formulae I, VI, VI-a, and VII, when X is —N(R^{a})—, or in some embodiments of Formula IV, V, or VI-b, R^{a} is hydrogen or —L^{R3}-R³.

In some embodiments, R^{a} is -L^{R3}-R³. In some embodiments of any of Formulae I, VIII, VIII-a, VIII-b, VIII-c, VIII-d, IX, IX-a, IX-b, IX-c, and IX-d, R^{a} is-L^{R3}-R³.

As described above, in some embodiments of any of Formulae I, VI, VI-a, and VII, when X is —N(R^{a})—, or in some embodiments of Formula IV, V, or VI-b, L^{R3} is a covalent bond or optionally substituted bivalent C₁₋₆ aliphatic.

In some embodiments of any of Formulae I, VIII, VIII-a, VIII-b, VIII-c, VIII-d, IX, IX-a, IX-b, IX-c, and IX-d, L^{R3} is a covalent bond.

In some embodiments, L^{R3} is a covalent bond. In some embodiments, L^{R3} is optionally substituted bivalent C₁₋₆ aliphatic. In some embodiments, L^{R3} is optionally substituted bivalent C₁₋₃ aliphatic. In some embodiments, L^{R3} is optionally substituted bivalent C₁₋₂ aliphatic. In some embodiments, L^{R3} is optionally substituted bivalent C₂ aliphatic. In some embodiments, L^{R3} is optionally substituted bivalent C₁ aliphatic.

In some embodiments, L^{R3} is a covalent bond or a bivalent C₁₋₆ aliphatic, wherein the bivalent C₁₋₆ aliphatic is optionally substituted with 1, 2, 3, or 4 independently selected R^{N} substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

As described above, in some embodiments of any of Formulae I, VI, VI-a, and VII, when X is —N(R^{a})—, or in some embodiments of Formula IV, V, or VI-b, R³ is hydrogen or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments of any of Formulae I, VIII, VIII-a, VIII-b, VIII-c, VIII-d, IX, IX-a, IX-b, IX-c, and IX-d, R³ is hydrogen or optionally substituted C₁₋₆ aliphatic.

In some embodiments, R³ is hydrogen or optionally substituted C₁₋₆ aliphatic.

In some embodiments, R³ is hydrogen or C₁₋₆ aliphatic, wherein the C₁₋₆ aliphatic is optionally substituted by 1, 2, 3, or 4 independently selected R^{3A} substituents.

In some embodiments, R³ is hydrogen or C₁₋₆ aliphatic, wherein the C₁₋₆ aliphatic is optionally substituted by 1, 2, or 3 independently selected R^{3A} substituents.

In some embodiments, R³ is hydrogen or C₁₋₆ aliphatic, wherein the C₁₋₆ aliphatic is optionally substituted by 1 or 2 independently selected R^{3A} substituents.

In some embodiments, R³ is hydrogen. In some embodiments, R³ is optionally substituted C₁₋₆ aliphatic. In some embodiments, R³ is optionally substituted C₁₋₃ aliphatic. In some embodiments, R³ is optionally substituted C₁₋₂ aliphatic. In some embodiments, R³ is optionally substituted C₂ aliphatic. In some embodiments, R³ is optionally substituted ethyl. In some embodiments, R³ is optionally substituted methyl.

In some embodiments, R³ is C₁-6 aliphatic, which is optionally substituted by 1, 2, 3, or 4 independently selected R^{3A} substituents. In some embodiments, R³ is C₁-3 aliphatic, which is optionally substituted by 1, 2, 3, or 4 independently selected R^{3A} substituents. In some embodiments, R³ is C₁-2 aliphatic, which is optionally substituted by 1, 2, 3, or 4 independently selected R^{3A} substituents. In some embodiments, R³ is optionally substituted C₂ aliphatic, which is optionally substituted by 1, 2, 3, or 4 independently selected R^{3A} substituents. In some embodiments, R³ is ethyl, which is optionally substituted by 1, 2, 3, or 4 independently selected R^{3A} substituents. In some embodiments, R³ is methyl, which is optionally substituted by 1, 2, or 3 independently selected R^{3A} substituents.

In some embodiments, each R^{3A} is an independently selected halogen. In some embodiments, each R^{3A} is fluoro.

In some embodiments, R^{a} is —CH₂CH₃ or —CH₂CF₂H. In some embodiments, R^{a} is —CH₂CH₃. In some embodiments, R^{a} is —CH₃, —CH₂CH₃, or —CH₂CF₂H.

In some embodiments of any of Formulae I, VIII, VIII-a, VIII-b, VIII-c, VIII-d, IX, IX-a, IX-b, IX-c, and IX-d, R^{a} is —CH₂CH₃.

In some embodiments, R⁴ and R⁵ are each independently hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or R⁴ and R⁵ are taken together with the carbon atom *C to which they are attached to form *C═O, *C═S, *C═NR^{L}, or an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.

In some embodiments, R⁴ is hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂R′, N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R⁴ is hydrogen.

In some embodiments, R⁵ is hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂R′, N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, and —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R⁵ is independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{4A} substituents; and
each R^{4A} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}), —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched alkylene)O—N(R^{∘})₂, and —(C₁₋₄ straight or branched) alkylene) C(O)O—N(R^{∘})₂.

In some embodiments of any of Formulae I, VIII, VIII-a, VIII-b, VIII-c, VIII-d, IX, IX-a, IX-b, IX-c, and IX-d, each R^{4A} is independently selected from C₁₋₆ aliphatic and —OC₁₋₆ aliphatic.

In some embodiments of any of Formulae I, VIII, VIII-a, VIII-b, VIII-c, VIII-d, IX, IX-a, IX-b, IX-c, and IX-d, each R^{4A} is independently selected from methyl and methoxy.

In some embodiments, R⁵ is hydrogen.

In some embodiments, R⁴ and R⁵ are taken together with the carbon atom *C to which they are attached to form *C═O, *C═S, *C═NR^{L}, or an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.

In some embodiments, R⁴ and R⁵ are taken together with the carbon atom *C to which they are attached to form *C═O or optionally substituted 3- to 7-membered saturated or partially unsaturated monocyclic carbocyclyl. In some embodiments, R⁴ and R⁵ are taken together with the carbon atom *C to which they are attached to form *C═O. In some embodiments, R⁴ and R⁵ are taken together with the carbon atom *C to which they are attached to form optionally substituted 3- to 7-membered saturated or partially unsaturated monocyclic carbocyclyl. In some embodiments, R⁴ and R⁵ are taken together with the carbon atom *C to which they are attached to form optionally substituted 3-membered saturated or partially unsaturated monocyclic carbocyclyl. In some embodiments, R⁴ and R⁵ are taken together with the carbon atom *C to which they are attached to form optionally substituted cyclopropyl.

As described above, in some embodiments of any of Formulae I, V, VI, VI-a, and VII, R⁴ and L^{D1}-R⁸ are taken together with the carbon to which they are attached to form an optionally substituted ring selected from phenyl, 5- to 6-membered monocyclic heteroaryl having 1-4 heteroatoms independently selected from nitrogen, oxygen, and sulfur. In some embodiments, R⁴ and L^{D1}-R⁸ are taken together with the carbon to which they are attached to form an optionally substituted 6-membered monocyclic heteroaryl having 1-2 nitrogen heteroatoms. In some embodiments, R⁴ and L^{D1}-R⁸ are taken together with the carbon to which they are attached to form optionally substituted pyrimidinyl.

In some embodiments, R^{L} is hydrogen, —CN, —OR^{L1}, or optionally substituted C₁₋₆ alkyl.

In some embodiments, R^{L} is hydrogen, —CN, —OR^{L1}, or C₁₋₆ alkyl, wherein the C₁₋₆ alkyl is optionally substituted with 1, 2, 3, or 4 independently selected RN substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, R^{L1} is hydrogen, C₁₋₆ alkyl, or C₁₋₆ haloalkyl.

In some embodiments, each L is independently a covalent bond or optionally substituted bivalent C₁₋₆ aliphatic.

In some embodiments, each L is a covalent bond.

In some embodiments, each L is independently a covalent bond or a bivalent C₁₋₆ aliphatic, wherein the bivalent C₁₋₆ aliphatic is optionally substituted with 1, 2, 3, or 4 independently selected RN substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, each RAI is independently halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, each RAI is independently selected from halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{B1} substituents; and
each R^{B1} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, each R^{A1} is independently optionally substituted C₁₋₆ aliphatic. In some embodiments, each R^{A1} is independently optionally substituted C₁₋₃ aliphatic. In some embodiments, each R^{A1} is independently optionally substituted C₁₋₂ aliphatic. In some embodiments, each R^{A1} is independently optionally substituted methyl.

In some embodiments, R⁶ and R⁷ are each independently hydrogen, halogen, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or R⁶ and R⁷ are taken together with the carbon to which they are attached to form an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.

In some embodiments, R⁶ and R⁷ are each independently hydrogen, halogen, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{6A} substituents; or
R⁶ and R⁷ are taken together with the carbon to which they are attached to form a 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl having 1-3 heteroatoms independently selected from nitrogen, oxygen, and sulfur, or a 6- to 10-membered saturated or partially unsaturated bicyclic heterocyclyl having 1-4 heteroatoms independently selected from nitrogen, oxygen, and sulfur, wherein the 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl, and 6- to 10-membered saturated or partially unsaturated bicyclic heterocyclyl are each optionally substituted with 1, 2, 3, or 4 independently selected R^{6A} substituents; and
each R^{6A} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, R⁶ is hydrogen, halogen, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R⁶ is hydrogen, deuterium, or optionally substituted C₁₋₆ aliphatic. In some embodiments, R⁶ is hydrogen or optionally substituted C₁₋₆ aliphatic. In some embodiments, R⁶ is hydrogen or deuterium. In some embodiments, R⁶ is hydrogen. In some embodiments, R⁶ is deuterium. In some embodiments, R⁶ is optionally substituted C₁₋₆ aliphatic. In some embodiments, R⁶ is optionally substituted C₁₋₃ aliphatic. In some embodiments, R⁶ is optionally substituted C₁₋₂ aliphatic. In some embodiments, R⁶ is optionally substituted C₂ aliphatic. In some embodiments, R⁶ is optionally substituted ethyl. In some embodiments, R⁶ is —CH₂CF₃.

In some embodiments of any of Formulae I, VIII, VIII-a, VIII-b, VIII-c, VIII-d, IX, IX-a, IX-b, IX-c, and IX-d, R⁶ is hydrogen, deuterium, or optionally substituted C₁₋₆ aliphatic.

In some embodiments of any of Formulae I, VIII, VIII-a, VIII-b, VIII-c, VIII-d, IX, IX-a, IX-b, IX-c, and IX-d, R⁶ is hydrogen or deuterium.

In some embodiments, R⁷ is hydrogen, halogen, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R⁷ is hydrogen, deuterium, or optionally substituted C₁₋₆ aliphatic. In some embodiments, R⁷ is hydrogen or optionally substituted C₁₋₆ aliphatic. In some embodiments, R⁷ is hydrogen or deuterium. In some embodiments, R⁷ is hydrogen. In some embodiments, R⁷ is deuterium. In some embodiments, R⁷ is optionally substituted C₁₋₆ aliphatic. In some embodiments, R⁷ is optionally substituted C₁₋₃ aliphatic. In some embodiments, R⁷ is optionally substituted C₁₋₂ aliphatic. In some embodiments, R⁷ is optionally substituted C₂ aliphatic. In some embodiments, R⁷ is optionally substituted ethyl. In some embodiments, R⁷ is —CH₂CF₃.

In some embodiments of any of Formulae I, VIII, VIII-a, VIII-b, VIII-c, VIII-d, IX, IX-a, IX-b, IX-c, and IX-d, R⁷ is hydrogen, deuterium, or optionally substituted C₁₋₆ aliphatic.

In some embodiments of any of Formulae I, VIII, VIII-a, VIII-b, VIII-c, VIII-d, IX, IX-a, IX-b, IX-c, and IX-d, R⁷ is hydrogen or deuterium.

In some embodiments, R⁶ and R⁷ are taken together with the carbon to which they are attached to form an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.

In some embodiments, R⁶ and R⁷ are taken together with the carbon to which they are attached to form optionally substituted 3- to 7-membered saturated or partially unsaturated monocyclic carbocyclyl. In some embodiments, R⁶ and R⁷ are taken together with the carbon to which they are attached to form optionally substituted 3-membered saturated or partially unsaturated monocyclic carbocyclyl. In some embodiments, R⁶ and R⁷ are taken together with the carbon to which they are attached to form optionally substituted cyclopropyl.

In some embodiments, R⁶ and R⁷ are each hydrogen.

In some embodiments, R⁶ and R⁷ are each deuterium.

In some embodiments of any of Formulae I, VIII, VIII-a, VIII-b, VIII-c, VIII-d, IX, IX-a, IX-b, IX-c, and IX-d, R⁶ and R⁷ are each hydrogen.

In some embodiments of any of Formulae I, VIII, VIII-a, VIII-b, VIII-c, VIII-d, IX, IX-a, IX-b, IX-c, and IX-d, R⁶ and R⁷ are each deuterium.

In some embodiments of any of Formulae I, VIII, VIII-a, VIII-b, VIII-c, VIII-d, IX, IX-a, IX-b, IX-c, and IX-d, R⁶ and R⁷ are each hydrogen.

In some embodiments of any of Formulae I, VIII, VIII-a, VIII-b, VIII-c, VIII-d, IX, IX-a, IX-b, IX-c, and IX-d, R⁶ and R⁷ are each deuterium.

As described above, in some embodiments of any of Formulae I, II, II-a, II-a-i, III, IV, V, VI, and VI-b, D¹ is C-L^{D1}-R⁸ or N.

In some embodiments, D¹ is C-L^{D1}-R⁸. In some embodiments, D¹ is N.

As described above, in some embodiments of any of Formulae I, II, II-a, II-a-i, VI, VI-b, and VII, D¹ is S or NR, and D² is absent. In some embodiments, D¹ is S, and D² is absent.

As described above, in some embodiments of any of Formulae I, II, II-a, II-a-i, III, IV, V, VI, and VI-b, L^{D1} is a covalent bond or optionally substituted bivalent C₁₋₆ aliphatic.

In some embodiments, L^{D1} is a covalent bond or a bivalent C₁₋₆ aliphatic, wherein the bivalent C₁₋₆ aliphatic is optionally substituted with 1, 2, 3, or 4 independently selected RN substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, L^{D1} is a covalent bond. In some embodiments, L^{D1} is optionally substituted bivalent C₁₋₆ aliphatic. In some embodiments, L^{D1} is optionally substituted bivalent C₁₋₃ aliphatic. In some embodiments, L^{D1} is optionally substituted bivalent C₁₋₂ aliphatic. In some embodiments, L^{D1} is optionally substituted bivalent C₁ aliphatic. In some embodiments, L^{D1} is —CH₂—.

In some embodiments, R⁸ is selected from hydrogen, halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, R⁸ is selected from hydrogen, halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{8A} substituents; and
each R^{8A} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, C(S)SR^{∘}, —SC(S)SR^{∘}, —(CH₂)₀₋₄OC(O)NR^{∘})₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR∘)NR^{∘}₂, —(CH₂)₀₋₄S(O)R^{∘}, —N(R^{∘})S(O)₂NR^{∘})₂, —N(R^{∘})S(O)₂R^{∘}, —N(R^{∘})S(O)(═NR^{∘})R^{∘}, —N(OR^{∘})R^{∘}, —C(NH)NR^{∘})₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘}2, and —(C₁₋₄ straight or branched) alkylene) C(O)O—N(R^{∘})₂.

In some embodiments, R⁸ is hydrogen, halogen, or optionally substituted 3- to 7-membered saturated or partially unsaturated monocyclic carbocyclyl. In some embodiments, R⁸ is hydrogen or optionally substituted 3- to 7-membered saturated or partially unsaturated monocyclic carbocyclyl. In some embodiments, R⁸ is hydrogen. In some embodiments, R⁸ is halogen. In some embodiments, R⁸ is —F or —Cl. In some embodiments, R⁸ is —F. In some embodiments, R⁸ is —C1. In some embodiments, R⁸ is optionally substituted 3- to 7-membered saturated or partially unsaturated monocyclic carbocyclyl. In some embodiments, R⁸ is optionally substituted 3-membered saturated or partially unsaturated monocyclic carbocyclyl. In some embodiments, R⁸ is optionally substituted cyclopropyl.

In some embodiments, C-L^{D1}-R⁸ is selected from —CH, —CF, —CCl, —CCHF₂, —COCH₃, —C-cyclopropyl, —C(hydroxymethyl), —C(cyanomethyl), and —C(methoxymethyl).

In some embodiments, D² is absent, C-L^{D2}-R⁹, or N. In some embodiments, D² is absent, C-L^{D2}-R⁹, or N, wherein when D¹ is S or NR, D² is absent.

In some embodiments of any of Formulae I, VIII, VIII-a, VIII-b, VIII-c, VIII-d, IX, IX-a, IX-b, IX-c, and IX-d, D² is C-L^{D2}-R⁹. In some embodiments of any of Formulae I, VIII, VIII-a, VIII-b, VIII-c, VIII-d, IX, IX-a, IX-b, IX-c, and IX-d, L^{D2} is a covalent bond. In some embodiments of any of Formulae I, VIII, VIII-a, VIII-b, VIII-c, VIII-d, IX, IX-a, IX-b, IX-c, and IX-d, R⁹ is hydrogen. In some embodiments of any of Formulae I, VIII, VIII-a, VIII-b, VIII-c, VIII-d, IX, IX-a, IX-b, IX-c, and IX-d, D² is CH. In some embodiments of any of Formulae I, VIII, VIII-a, VIII-b, VIII-c, VIII-d, IX, IX-a, IX-b, IX-c, and IX-d, D² is N.

In some embodiments, D² is C-L^{D2}-R⁹. In some embodiments, D² is N.

In some embodiments, L^{D2} is a covalent bond or optionally substituted bivalent C₁₋₆ aliphatic.

In some embodiments, L^{D2} is a covalent bond or a bivalent C₁₋₆ aliphatic, wherein the bivalent C₁₋₆ aliphatic is optionally substituted with 1, 2, 3, or 4 independently selected RN substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, L^{D2} is a covalent bond. In some embodiments, L^{D2} is optionally substituted bivalent C₁₋₆ aliphatic. In some embodiments, L^{D2} is optionally substituted bivalent C₁₋₃ aliphatic. In some embodiments, L^{D2} is optionally substituted bivalent C₁₋₂ aliphatic. In some embodiments, L^{D2} is optionally substituted bivalent C₁ aliphatic. In some embodiments, L^{D2} is —CH₂—.

In some embodiments, D² is CH.

In some embodiments, D³ is CR¹⁰ or N.

In some embodiments, D³ is CR¹⁰.

In some embodiments, R⁹ and R¹⁰ are each independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R⁹ and R¹⁰ are each independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{9A} substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, R⁹ is selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R⁹ is hydrogen or —CN. In some embodiments, R⁹ is hydrogen. In some embodiments, R⁹ is —CN.

In some embodiments, R¹⁰ is selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, and —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur.

In some embodiments, R¹⁰ is hydrogen. In some embodiments, R¹⁰ is halogen. In some embodiments, R¹⁰ is —F.

In some embodiments, D³ is —CH. In some embodiments, D³ is —CF.

In some embodiments of any of Formulae I, VIII, VIII-a, VIII-b, VIII-c, VIII-d, IX, IX-a, IX-b, IX-c, and IX-d, D³ is CR¹⁰. In some embodiments of any of Formulae I, VIII, VIII-a, VIII-b, VIII-c, VIII-d, IX, IX-a, IX-b, IX-c, and IX-d, R¹⁰ is hydrogen or halogen. In some embodiments of any of Formulae I, VIII, VIII-a, VIII-b, VIII-c, VIII-d, IX, IX-a, IX-b, IX-c, and IX-d, R¹⁰ is fluoro. In some embodiments of any of Formulae I, VIII, VIII-a, VIII-b, VIII-c, VIII-d, IX, IX-a, IX-b, IX-c, and IX-d, D³ is CF.

In some embodiments, Ring B is 3- to 7-membered saturated or partially unsaturated monocyclic heterocyclylene having 1-3 heteroatoms independently selected from nitrogen, oxygen, and sulfur, 6- to 10-membered saturated or partially unsaturated bicyclic heterocyclylene having 1-4 heteroatoms independently selected from nitrogen, oxygen, and sulfur, or 9- to 16-membered saturated or partially unsaturated polycyclic heterocyclylene having 1-5 heteroatoms independently selected from nitrogen, oxygen, and sulfur.

In some embodiments, Ring B is 3- to 7-membered saturated or partially unsaturated monocyclic heterocyclylene having 1-3 heteroatoms independently selected from nitrogen, oxygen, and sulfur. In some embodiments, Ring B is 3- to 7-membered saturated or partially unsaturated monocyclic heterocyclylene having 1-3 heteroatoms independently selected from nitrogen, oxygen, and sulfur. In some embodiments, Ring B is 5- to 6-membered saturated or partially unsaturated monocyclic heterocyclylene having 1-3 heteroatoms independently selected from nitrogen, oxygen, and sulfur. In some embodiments, Ring B is 6-membered saturated or partially unsaturated monocyclic heterocyclylene having 1-3 heteroatoms independently selected from nitrogen, oxygen, and sulfur. In some embodiments, Ring B is 6-membered saturated or partially unsaturated monocyclic heterocyclylene having 1-3 nitrogen atoms. In some embodiments, Ring B is 6-membered saturated or partially unsaturated monocyclic heterocyclylene having 1-2 nitrogen atoms. In some embodiments, Ring B is piperazinylene (i.e., a piperazinyl ring). In some embodiments, Ring B is piperidinylene (i.e., piperidinyl).

In some embodiments, Ring B is 6- to 10-membered saturated or partially unsaturated bicyclic heterocyclylene having 1-4 heteroatoms independently selected from nitrogen, oxygen, and sulfur. In some embodiments, Ring B is 7- to 9-membered saturated or partially unsaturated bicyclic heterocyclylene having 1-4 heteroatoms independently selected from nitrogen, oxygen, and sulfur. In some embodiments, Ring B is 7-membered saturated or partially unsaturated bicyclic heterocyclylene having 1-4 heteroatoms independently selected from nitrogen, oxygen, and sulfur. In some embodiments, Ring B is 7-membered saturated or partially unsaturated bicyclic heterocyclylene having 1-4 nitrogen atoms. In some embodiments, Ring B is 7-membered saturated or partially unsaturated bicyclic heterocyclylene having 1-2 nitrogen atoms. In some embodiments, Ring B is 8-membered saturated or partially unsaturated bicyclic heterocyclylene having 1-4 heteroatoms independently selected from nitrogen, oxygen, and sulfur. In some embodiments, Ring B is 8-membered saturated or partially unsaturated bicyclic heterocyclylene having 1-4 nitrogen atoms. In some embodiments, Ring B is 8-membered saturated or partially unsaturated bicyclic heterocyclylene having 1-3 nitrogen atoms. In some embodiments, Ring B is 8-membered saturated or partially unsaturated bicyclic heterocyclylene having 1-2 nitrogen atoms. In some embodiments, Ring B is 9-membered saturated or partially unsaturated bicyclic heterocyclylene having 1-4 heteroatoms independently selected from nitrogen, oxygen, and sulfur. In some embodiments, Ring B is 9-membered saturated or partially unsaturated bicyclic heterocyclylene having 1-4 nitrogen atoms. In some embodiments, Ring B is 9-membered saturated or partially unsaturated bicyclic heterocyclylene having 1-2 nitrogen atoms.

In some embodiments, Ring B is 9- to 16-membered saturated or partially unsaturated polycyclic heterocyclylene having 1-5 heteroatoms independently selected from nitrogen, oxygen, and sulfur. In some embodiments, Ring B is 9-membered saturated or partially unsaturated polycyclic heterocyclylene having 1-5 heteroatoms independently selected from nitrogen, oxygen, and sulfur. In some embodiments, Ring B is 9-membered saturated or partially unsaturated polycyclic heterocyclylene having 1-5 nitrogen atoms. In some embodiments, Ring B is 9-membered saturated or partially unsaturated polycyclic heterocyclylene having 1-2 nitrogen atoms.

In some embodiments,

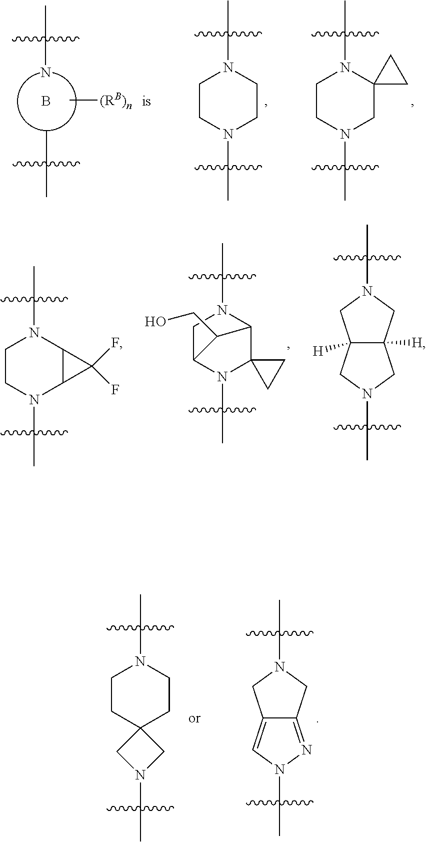

In some embodiments,

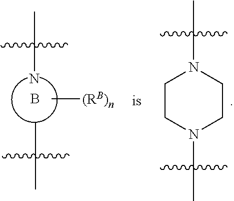

In some embodiments of any of Formulae I, VIII, VIII-a, VIII-b, VIII-c, VIII-d, IX, IX-a, IX-b, IX-c, and IX-d, Ring B is 3- to 7-membered saturated or partially unsaturated monocyclic heterocyclylene having 1-3 heteroatoms independently selected from nitrogen, oxygen, and sulfur.

In some embodiments of any of Formulae I, VIII, VIII-a, VIII-b, VIII-c, VIII-d, IX, IX-a, IX-b, IX-c, and IX-d, Ring B is 6-membered saturated or partially unsaturated monocyclic heterocyclylene having 1-3 nitrogen atoms.

In some embodiments of any of Formulae I, VIII, VIII-a, VIII-b, VIII-c, VIII-d, IX, IX-a, IX-b, IX-c, and IX-d, n is 0 or 2. In some embodiments of any of Formulae I, VIII, VIII-a, VIII-b, VIII-c, VIII-d, IX, IX-a, IX-b, IX-c, and IX-d, n is 0.

In some embodiments of any of Formulae I, VIII, VIII-a, VIII-b, VIII-c, VIII-d, IX, IX-a, IX-b, IX-c, and IX-d,

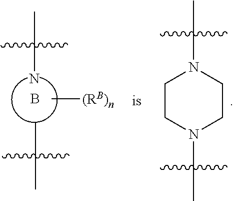

In some embodiments, Ring C is phenyl, 8- to 10-membered bicyclic aryl, 10- to 14-membered polycyclic aryl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 10- to 16-membered polycyclic heteroaryl having 1-5 heteroatoms independently selected from nitrogen, oxygen, and sulfur, or 6- to 10-membered saturated or partially unsaturated bicyclic heterocyclyl having 1-4 heteroatoms independently selected from nitrogen, oxygen, and sulfur.

In some embodiments, Ring C is phenyl.

In some embodiments, Ring C is 5- to 6-membered monocyclic heteroaryl having 1-4 heteroatoms independently selected from nitrogen, oxygen, and sulfur. In some embodiments, Ring C is 6-membered monocyclic heteroaryl having 1-4 nitrogen atoms. In some embodiments, Ring C is 6-membered monocyclic heteroaryl having 1-2 nitrogen atoms. In some embodiments, Ring C is pyridyl.

In some embodiments, Ring C is 6- to 10-membered saturated or partially unsaturated bicyclic heterocyclyl having 1-4 heteroatoms independently selected from nitrogen, oxygen, and sulfur. In some embodiments, Ring C is 9-membered saturated or partially unsaturated bicyclic heterocyclyl having 1-4 heteroatoms independently selected from nitrogen, oxygen, and sulfur. In some embodiments, Ring C is 9-membered saturated or partially unsaturated bicyclic heterocyclyl having 1-4 nitrogen atoms. In some embodiments, Ring C is 9-membered saturated or partially unsaturated bicyclic heterocyclyl having 1-2 nitrogen atoms.

In some embodiments,

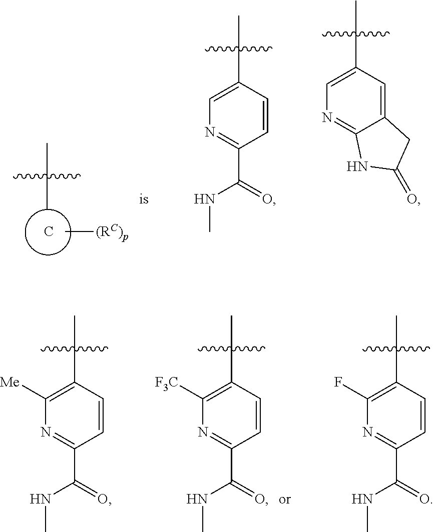

In some embodiments, Ring C is

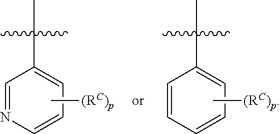

In some embodiments, Ring C is

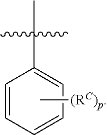

In some embodiments of any of Formulae I, VIII, VIII-a, VIII-b, VIII-c, VIII-d, IX, IX-a, IX-b, IX-c, and IX-d, Ring C is

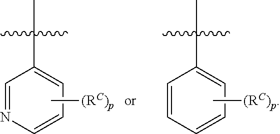

In some embodiments of any of Formulae I, VIII, VIII-a, VIII-b, VIII-c, VIII-d, IX, IX-a, IX-b, IX-c, and IX-d, Ring C is

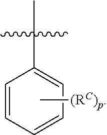

In some embodiments, Ring C is

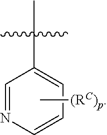

In some embodiments, Ring C is selected from

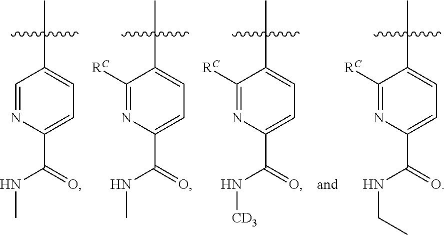

In some embodiments, each R^{B} is independently -L^{RB}-R¹¹.

In some embodiments, each L^{RB} is independently a covalent bond or optionally substituted bivalent C₁₋₆ aliphatic.

In some embodiments, each L^{RB} is independently a covalent bond or a bivalent C₁₋₆ aliphatic, wherein the bivalent C₁₋₆ aliphatic is optionally substituted with 1, 2, 3, or 4 independently selected RN substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, L^{RB} is a covalent bond. In some embodiments, L^{RB} is independently optionally substituted bivalent C₁₋₆ aliphatic. In some embodiments, L^{RB} is independently optionally substituted bivalent C₁₋₃ aliphatic. In some embodiments, L^{RB} is independently optionally substituted bivalent C₁₋₂ aliphatic. In some embodiments, L^{RB} is independently optionally substituted bivalent C₁ aliphatic.

In some embodiments, L^{RB} is —CH₂—.

In some embodiments, each R^{C} is independently -L^{RC}-R¹².

In some embodiments, each L^{RC} is independently a covalent bond or optionally substituted bivalent C₁₋₆ aliphatic.

In some embodiments, each L^{RC} is independently a covalent bond or a bivalent C₁₋₆ aliphatic, wherein the bivalent C₁₋₆ aliphatic is optionally substituted with 1, 2, 3, or 4 independently selected RN substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, L^{RC} is a covalent bond.

In some embodiments, R¹¹ and R¹² are each independently halogen, ═O, —CN, —OR, —SR, —N(R)₂, —N(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or a RB and a R^{C} can be taken together with their intervening atoms to form Ring D fused with one or both of Ring B and Ring C.

In some embodiments, R¹¹ and R¹² are each independently selected from halogen, ═O, —CN, —OR, —SR, —N(R)₂, —N(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{11A} substituents; and
each R^{11A} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O) CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘}2, and —(C₁₋₄ straight or branched) alkylene) C(O)O—N(R^{∘})₂.

In some embodiments, each R¹¹ is independently halogen, ═O, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, R¹¹ is halogen. In some embodiments, R¹¹ is —F. In some embodiments, R¹¹ is-OR. In some embodiments, R¹¹ is-OH. In some embodiments, R¹¹ is optionally substituted C₁₋₆ aliphatic.

In some embodiments, each R¹² is independently halogen, ═O, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur.

In some embodiments, R¹² is —C(O)N(R)₂. In some embodiments, R¹² is —C(O)N(Me)₂. In some embodiments, R¹² is —O.

In some embodiments, R¹² is optionally substituted C₁₋₆ aliphatic. In some embodiments, R¹² is optionally substituted C₁₋₃ aliphatic. In some embodiments, R¹² is optionally substituted C₁₋₂ aliphatic. In some embodiments, R¹² is optionally substituted methyl. In some embodiments, R¹² is methyl substituted with 1-3 halogen atoms. In some embodiments, R¹² is methyl substituted with 1-3 fluorine atoms. In some embodiments, R¹² is methyl. In some embodiments, R¹² is —CF₃.

In some embodiments, R¹² is halogen. In some embodiments, R¹² is —F.

In some embodiments, each R¹² is independently selected from —C(O)NHCH₃, —C(O)NHCD₃, C(O)NHCH₂CH₂, methyl, trifluoromethyl, fluoro, and trideuteromethyl (—CD₃).

In some embodiments, each R^{C} is independently selected from —C(O)NHCH₃, —C(O)NHCD₃, C(O)NHCH₂CH₂, methyl, trifluoromethyl, fluoro, and trideuteromethyl (—CD₃).

In some embodiments, Ring C is substituted by one R^{C} substituent selected from —C(O)NHCH₃, —C(O)NHCD₃, C(O)NHCH₂CH₂, and optionally substituted by a second R^{C} substituent selected from methyl, trifluoromethyl, fluoro, and trideuteromethyl (—CD₃).

In some embodiments, Ring C is substituted by one R^{C} substituent selected from —C(O)NHCH₃, —C(O)NHCD₃, C(O)NHCH₂CH₂.

In some embodiments, Ring C is substituted by one R^{C} substituent selected from —C(O)NHCH₃, —C(O)NHCD₃, C(O)NHCH₂CH₂, and a second R^{C} substituent selected from methyl, trifluoromethyl, fluoro, and trideuteromethyl (—CD₃).

In some embodiments, Ring C is substituted by one or two R^{C} substituents selected from methyl and fluoro, and a third R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments, Ring C is substituted by one R^{C} substituent which is methyl, and a second R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments, Ring C is substituted by one R^{C} substituent which is fluoro, and a second R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments, Ring C is substituted by two R^{C} substituents which are each fluoro, and a third R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments, Ring C is phenyl or pyridinyl, each of which is substituted by one or two R^{C} substituents selected from methyl and fluoro, and a third R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments, Ring C is phenyl or pyridinyl, each of which is substituted by one R^{C} substituent which is methyl, and a second R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments, Ring C is phenyl or pyridinyl, substituted by one RC substituent which is fluoro, and a second R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments, Ring C is phenyl, which is substituted by two RC substituents which are each fluoro, and a third R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments,

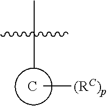

is selected from

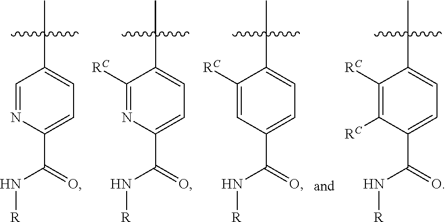

In some embodiments of the previous embodiment:
each R^{C} is independently selected from methyl and fluoro; and
each R is independently selected from methyl, cyclopropyl, methoxy, cyclopropylmethoxy, cyanocyclopropyl, cyanomethylcyclopropyl, hydroxymethylcyclopropyl, methoxymethylcyclopropyl, cyclobutyl, cyanocyclobutyl, hydroxycyclobutyl, difluorocyclobutyl, cyanocyclohexyl, tetrahydropyranyl, tetrahydrofuranyl, 3-oxabicyclo[3.1.0]hexanyl, methylpyrrolidinyl, and methylpiperidinyl.

In some embodiments,

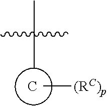

is selected from

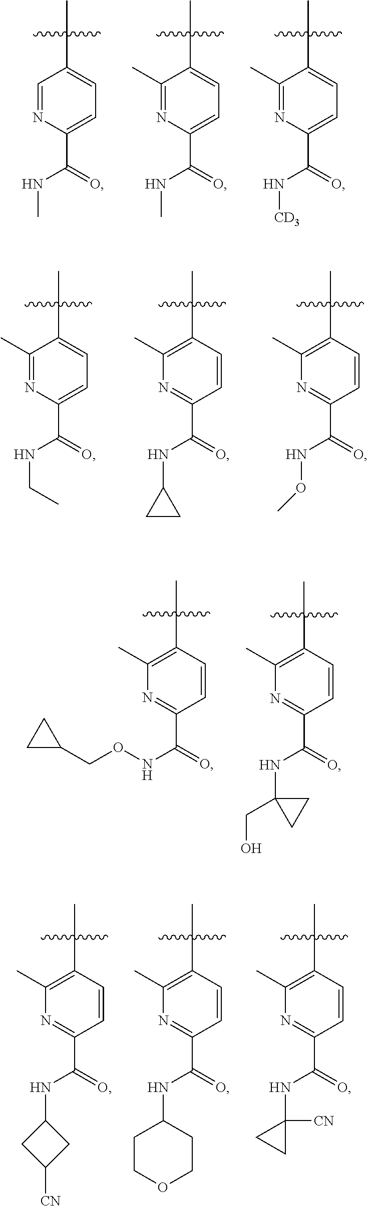

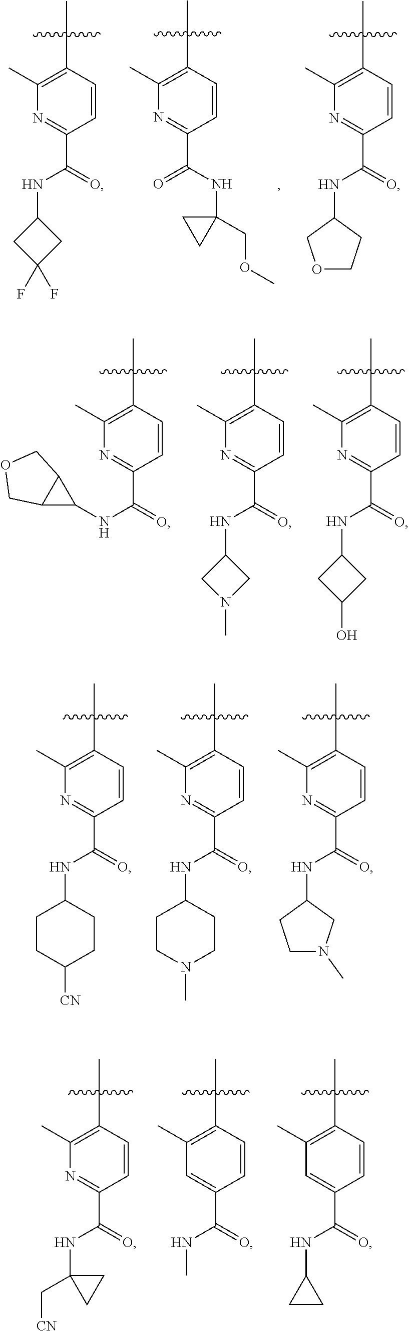

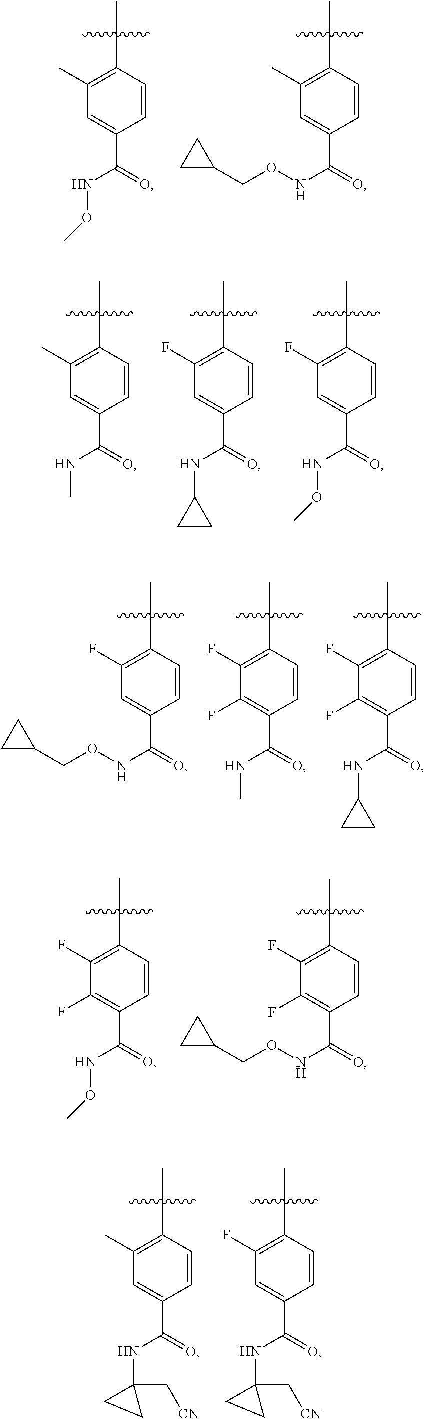

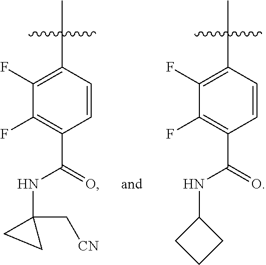 [SMILES: *c1ccc(C(=O)NC2(CC#N)CC2)c(F)c1F ; "and" ; *c1ccc(C(=O)NC2CCC2)c(F)c1F]

In some embodiments,

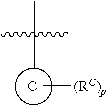

is selected from

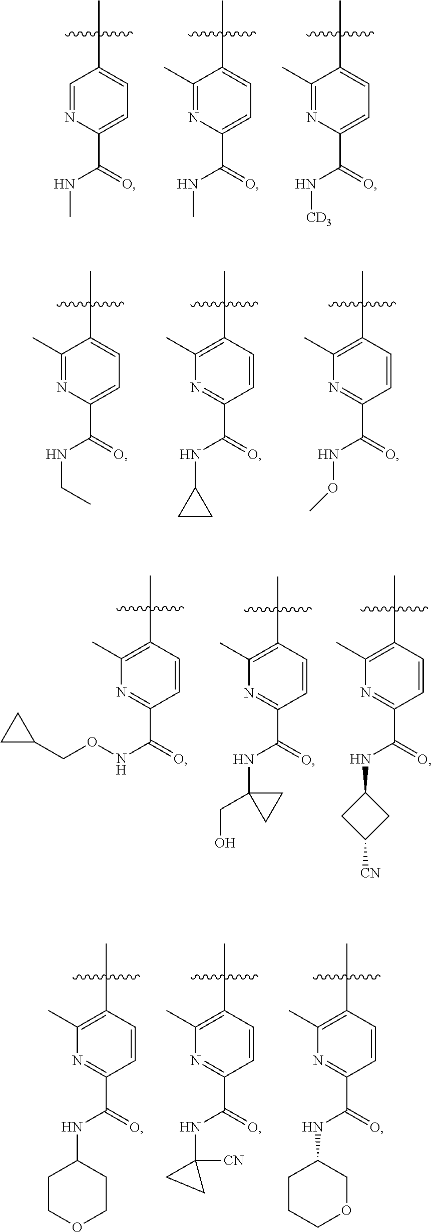

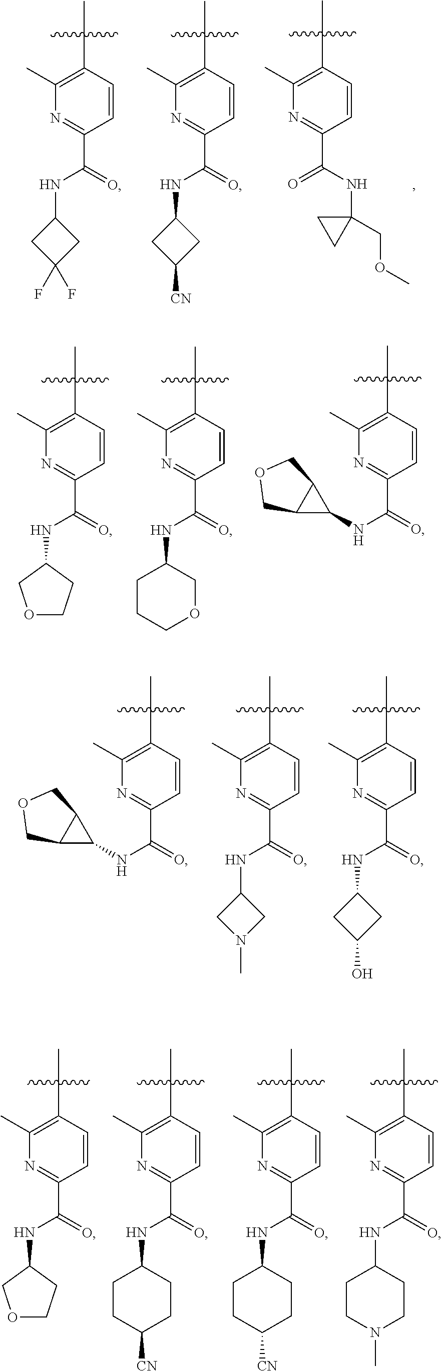

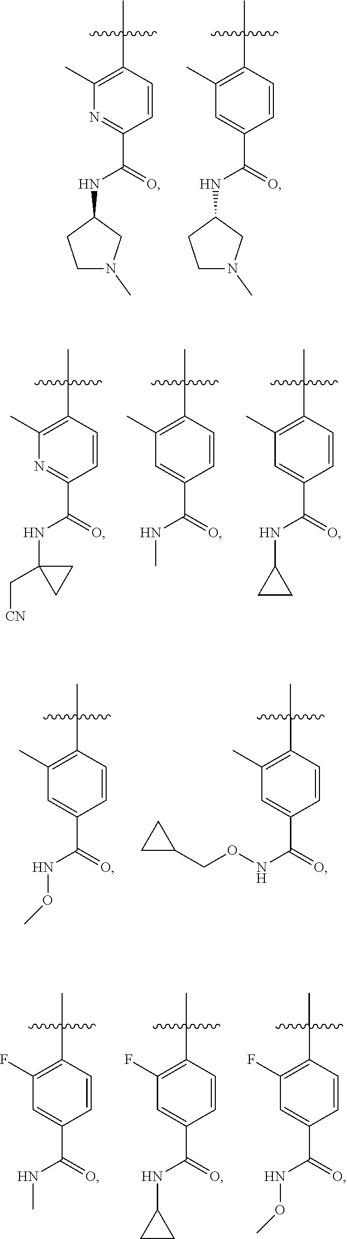

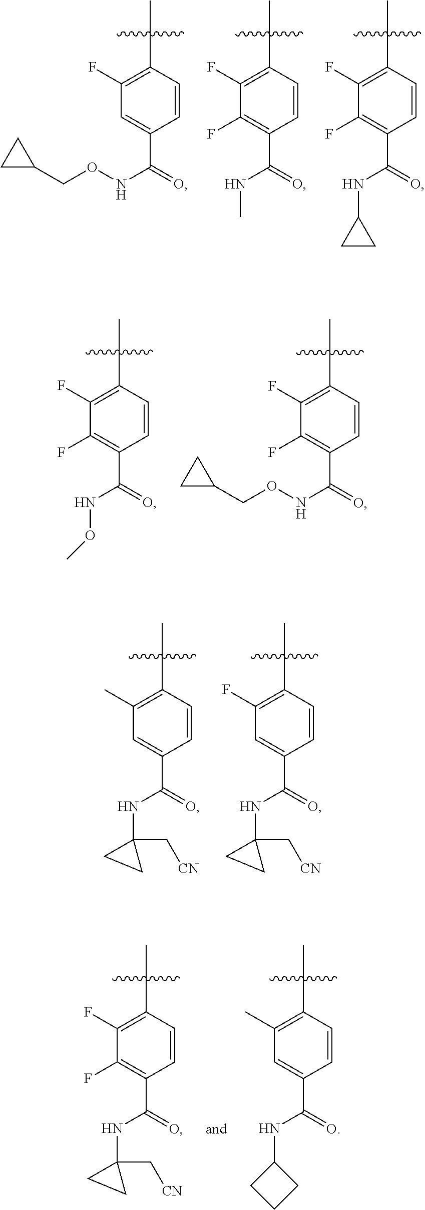

In some embodiments of any of Formulae I, VIII, VIII-a, VIII-b, VIII-c, VIII-d, IX, IX-a, IX-b, IX-c, and IX-d, Ring C is 5- to 6-membered monocyclic heteroaryl having 1-4 heteroatoms independently selected from nitrogen, oxygen, and sulfur.

In some embodiments of any of Formulae I, VIII, VIII-a, VIII-b, VIII-c, VIII-d, IX, IX-a, IX-b, IX-c, and IX-d, Ring C is 6-membered monocyclic heteroaryl having 1-2 nitrogen atoms.

In some embodiments of any of Formulae I, VIII, VIII-a, VIII-b, VIII-c, VIII-d, IX, IX-a, IX-b, IX-c, and IX-d, Ring C is phenyl.

In some embodiments of any of Formulae I, VIII, VIII-a, VIII-b, VIII-c, VIII-d, IX, IX-a, IX-b, IX-c, and IX-d, Ring C is

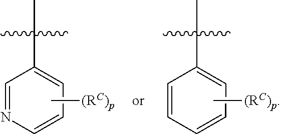

In some embodiments of any of Formulae I, VIII, VIII-a, VIII-b, VIII-c, VIII-d, IX, IX-a, IX-b, IX-c, and IX-d, p is 1, 2, or 3. In some embodiments of any of Formulae I, VIII, VIII-a, VIII-b, VIII-c, VIII-d, IX, IX-a, IX-b, IX-c, and IX-d, p is 2. In some embodiments of any of Formulae I, VIII, VIII-a, VIII-b, VIII-c, VIII-d, IX, IX-a, IX-b, IX-c, and IX-d, p is 3. In some embodiments of any of Formulae I, VIII, VIII-a, VIII-b, VIII-c, VIII-d, IX, IX-a, IX-b, IX-c, and IX-d,

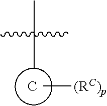

is selected from

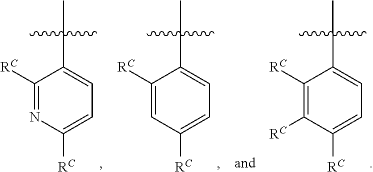

In some embodiments of any of Formulae I, VIII, VIII-a, VIII-b, VIII-c, VIII-d, IX, IX-a, IX-b, IX-c, and IX-d, each L^{RC} is a covalent bond.

In some embodiments of any of Formulae I, VIII, VIII-a, VIII-b, VIII-c, VIII-d, IX, IX-a, IX-b, IX-c, and IX-d, each R¹² is independently selected from halogen, C₁₋₆ aliphatic, —C(O)N(R)₂, and —C(O)NR(OR).

In some embodiments of any of Formulae I, VIII, VIII-a, VIII-b, VIII-c, VIII-d, IX, IX-a, IX-b, IX-c, and IX-d, each R¹² is independently selected from fluoro, methyl, —C(O)NHR, and —C(O)NH(OR).

In some embodiments of any of Formulae I, VIII, VIII-a, VIII-b, VIII-c, VIII-d, IX, IX-a, IX-b, IX-c, and IX-d, each R is independently selected from hydrogen, C₁₋₆ aliphatic, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.
each R^{C} is independently selected from methyl and fluoro; and
each R is independently selected from hydrogen, C₁₋₆ aliphatic, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.

In some embodiments of any of Formulae I, VIII, VIII-a, VIII-b, VIII-c, VIII-d, IX, IX-a, IX-b, IX-c, and IX-d, each R is independently selected from methyl, cyclopropyl, methoxy, cyclopropylmethoxy, cyanocyclopropyl, cyanomethylcyclopropyl, hydroxymethylcyclopropyl, methoxymethylcyclopropyl, cyclobutyl, cyanocyclobutyl, hydroxycyclobutyl, difluorocyclobutyl, cyanocyclohexyl, tetrahydropyranyl, tetrahydrofuranyl, 3-oxabicyclo[3.1.0]hexanyl, methylpyrrolidinyl, and methylpiperidinyl.

In some embodiments of any of Formulae I, VIII, VIII-a, VIII-b, VIII-c, VIII-d, IX, IX-a, IX-b, IX-c, and IX-d, Ring C is substituted by one or two R^{C} substituents selected from methyl and fluoro, and a third R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments of any of Formulae I, VIII, VIII-a, VIII-b, VIII-c, VIII-d, IX, IX-a, IX-b, IX-c, and IX-d, Ring C is substituted by one R^{C} substituent which is methyl, and a second R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments of any of Formulae I, VIII, VIII-a, VIII-b, VIII-c, VIII-d, IX, IX-a, IX-b, IX-c, and IX-d, Ring C is substituted by one R^{C} substituent which is fluoro, and a second R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments of any of Formulae I, VIII, VIII-a, VIII-b, VIII-c, VIII-d, IX, IX-a, IX-b, IX-c, and IX-d, Ring C is substituted by two R^{C} substituents which are each fluoro, and a third R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments of any of Formulae I, VIII, VIII-a, VIII-b, VIII-c, VIII-d, IX, IX-a, IX-b, IX-c, and IX-d, Ring C is phenyl or pyridinyl, each of which is substituted by one or two R^{C} substituents selected from methyl and fluoro, and a third R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments of any of Formulae I, VIII, VIII-a, VIII-b, VIII-c, VIII-d, IX, IX-a, IX-b, IX-c, and IX-d, Ring C is phenyl or pyridinyl, each of which is substituted by one R^{C} substituent which is methyl, and a second R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments of any of Formulae I, VIII, VIII-a, VIII-b, VIII-c, VIII-d, IX, IX-a, IX-b, IX-c, and IX-d, Ring C is phenyl or pyridinyl, substituted by one RC substituent which is fluoro, and a second R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments of any of Formulae I, VIII, VIII-a, VIII-b, VIII-c, VIII-d, IX, IX-a, IX-b, IX-c, and IX-d, Ring C is phenyl, which is substituted by two RC substituents which are each fluoro, and a third R^{C} substituent selected from methylcarbamyl, cyclopropylcarbamyl, methoxycarbamyl, (cyclopropylmethoxy)carbamyl, (cyanocyclopropyl)carbamyl, (cyanomethylcyclopropyl)carbamyl, (hydroxymethylcyclopropyl)carbamyl, (methoxymethylcyclopropyl)carbamyl, cyclobutylcarbamyl, (cyanocyclobutyl)carbamyl, (hydroxycyclobutyl)carbamyl, (difluorocyclobutyl)carbamyl, (cyanocyclohexyl)carbamyl, tetrahydropyranylcarbamyl, tetrahydrofuranylcarbamyl, 3-oxabicyclo[3.1.0]hexanylcarbamyl, (methylpyrrolidinyl)carbamyl, and (methylpiperidinyl)carbamyl.

In some embodiments of any of Formulae I, VIII, VIII-a, VIII-b, VIII-c, VIII-d, IX, IX-a, IX-b, IX-c, and IX-d,

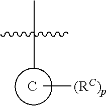

is selected from

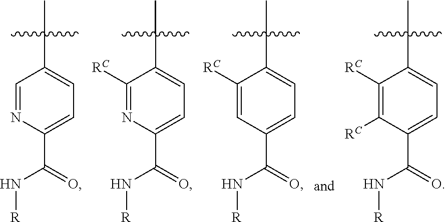

In some embodiments of the previous embodiment:
each R^{C} is independently selected from methyl and fluoro; and
each R is independently selected from methyl, cyclopropyl, methoxy, cyclopropylmethoxy, cyanocyclopropyl, cyanomethylcyclopropyl, hydroxymethylcyclopropyl, methoxymethylcyclopropyl, cyclobutyl, cyanocyclobutyl, hydroxycyclobutyl, difluorocyclobutyl, cyanocyclohexyl, tetrahydropyranyl, tetrahydrofuranyl, 3-oxabicyclo[3.1.0]hexanyl, methylpyrrolidinyl, and methylpiperidinyl.

In some embodiments of any of Formulae I, VIII, VIII-a, VIII-b, VIII-c, VIII-d, IX, IX-a, IX-b, IX-c, and IX-d,

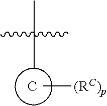

is selected from

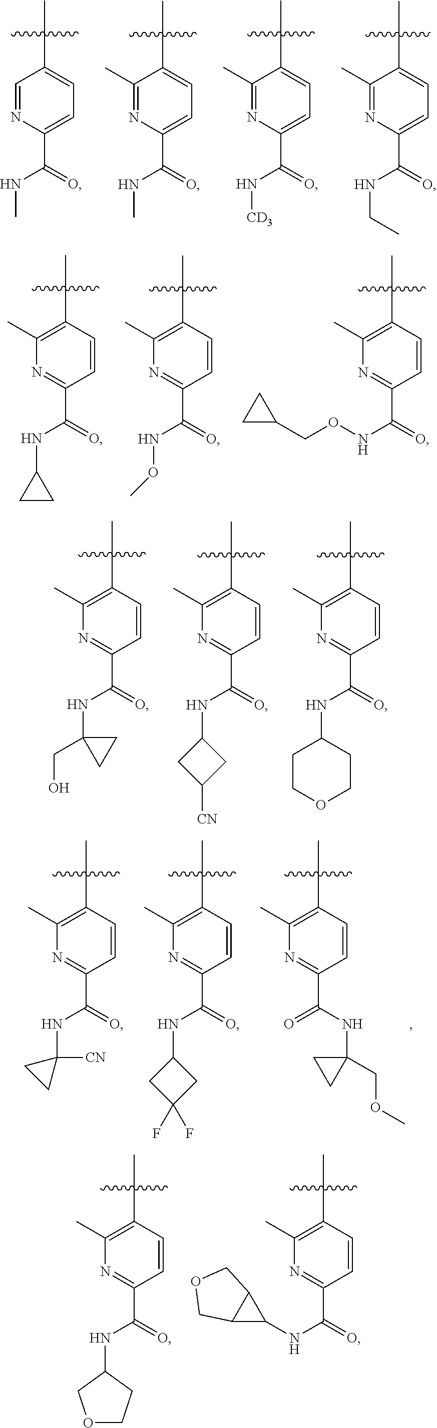

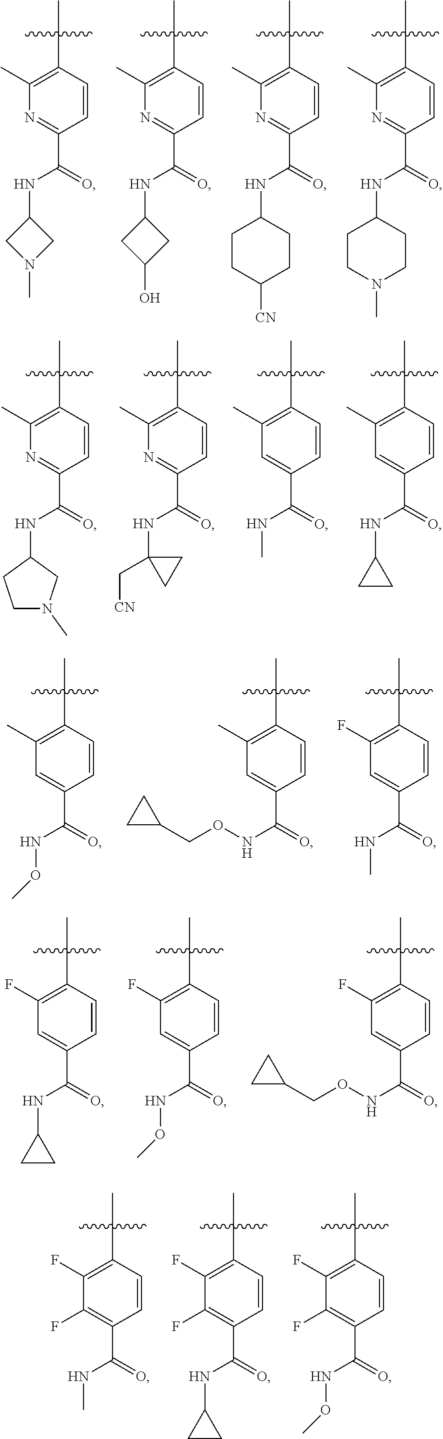

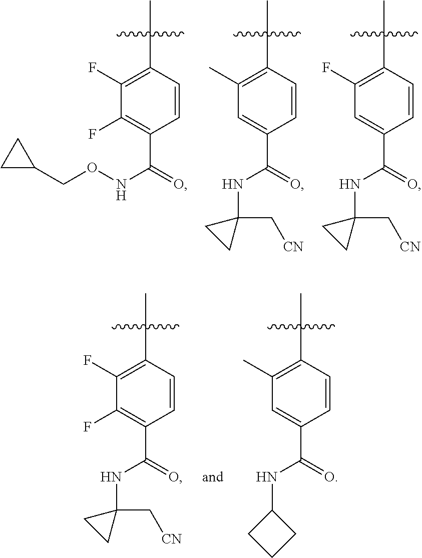

In some embodiments of any of Formulae I, VIII, VIII-a, VIII-b, VIII-c, VIII-d, IX, IX-a, IX-b, IX-c, and IX-d,

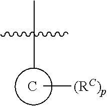

is selected from

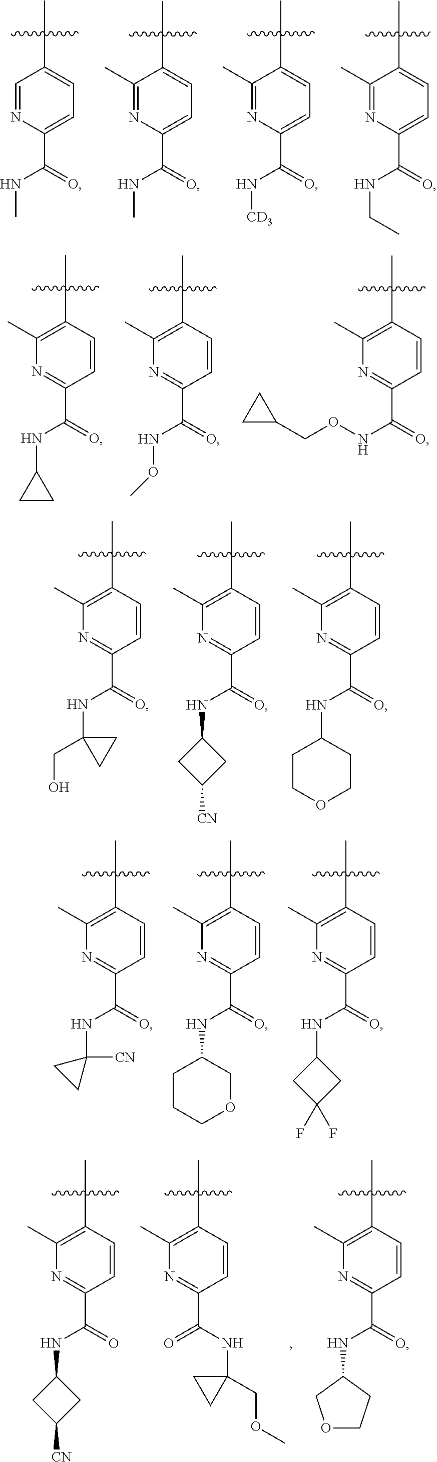

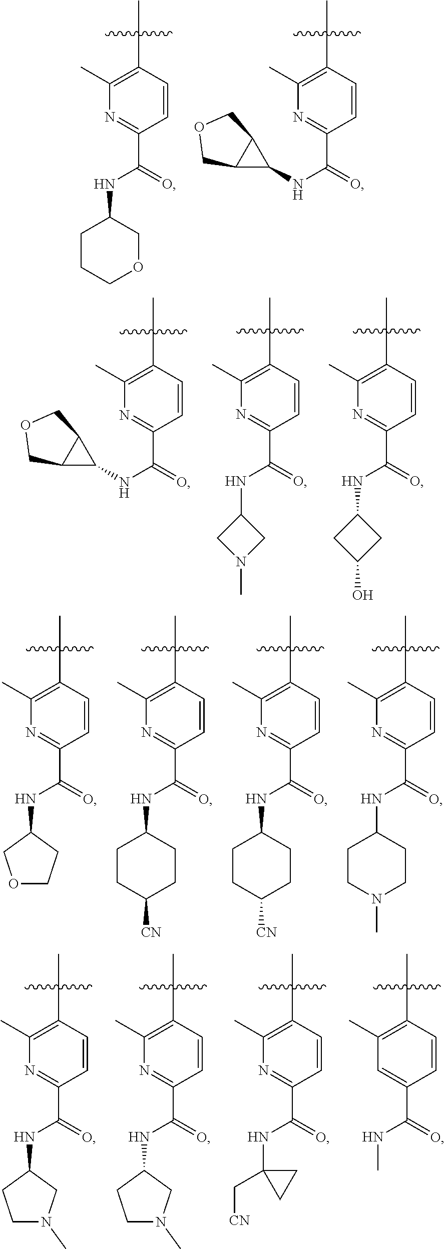

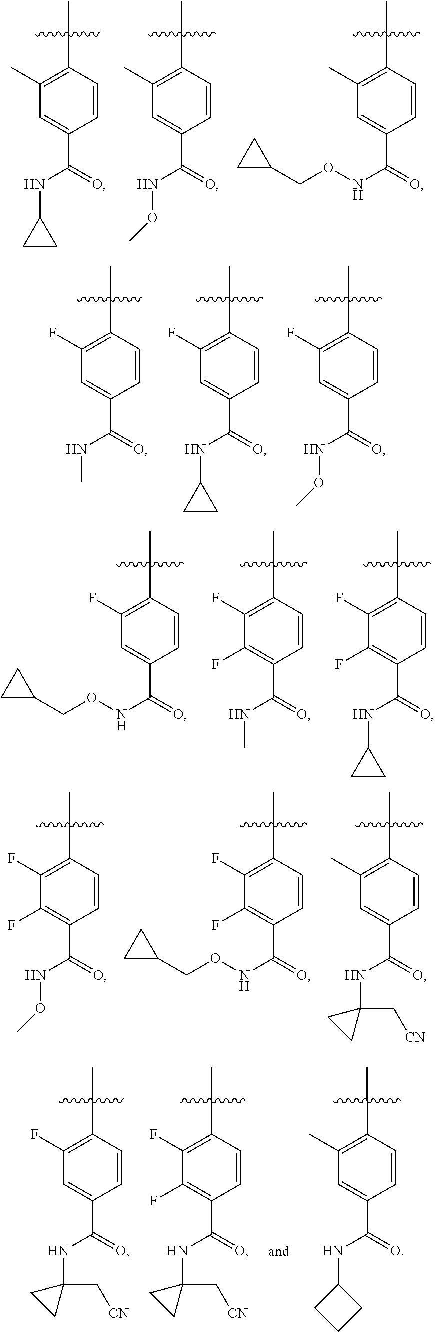

In some embodiments, Ring C is selected from

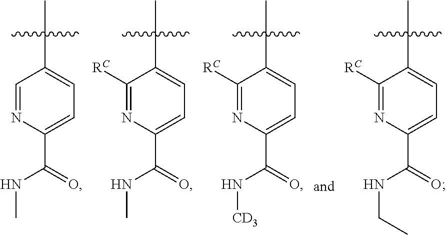

and

R^{C} is selected from methyl, trifluoromethyl, fluoro, and trideuteromethyl (—CD₃).

In some embodiments, a RB and a R^{C} are taken together with their intervening atoms to form Ring D fused with one or both of Ring B and Ring C.

In some embodiments, Ring D is an optionally substituted ring selected from 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 6- to 8-membered saturated or partially unsaturated bicyclic heterocyclyl having 1-4 heteroatoms independently selected from nitrogen, oxygen, and sulfur, phenyl, and 5- to 6-membered monocyclic heteroaryl having 1-4 heteroatoms independently selected from nitrogen, oxygen, and sulfur. In some embodiments, Ring D is an optionally substituted ring selected from 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 5- to 6-membered monocyclic heteroaryl having 1-4 heteroatoms independently selected from nitrogen, oxygen, and sulfur.

In some embodiments, Ring D is selected from 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 6- to 8-membered saturated or partially unsaturated bicyclic heterocyclyl having 1-4 heteroatoms independently selected from nitrogen, oxygen, and sulfur, phenyl, and 5- to 6-membered monocyclic heteroaryl having 1-4 heteroatoms independently selected from nitrogen, oxygen, and sulfur, wherein the 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, 6- to 8-membered saturated or partially unsaturated bicyclic heterocyclyl, phenyl, and 5- to 6-membered monocyclic heteroaryl are each optionally substituted with 1, 2, 3, or 4 independently selected R^{D1} substituents; and
each R^{D1} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘}2, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘}R^{∘}, —C(O)C(O)R^{∘}, —C(O) CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR∘)NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘}2, and —(C₁₋₄ straight or branched) alkylene) C(O)O—N(R^{∘})₂.

In some embodiments, Ring D is optionally substituted 3- to 7-membered saturated or partially unsaturated monocyclic carbocyclyl. In some embodiments, Ring D is optionally substituted 5- to 6-membered saturated or partially unsaturated monocyclic carbocyclyl. In some embodiments, Ring D is optionally substituted 5-membered saturated or partially unsaturated monocyclic carbocyclyl.

In some embodiments, Ring D is optionally substituted 3- to 7-membered saturated or partially unsaturated monocyclic heterocyclyl having 1-3 heteroatoms independently selected from nitrogen, oxygen, and sulfur. In some embodiments, Ring D is optionally substituted 5- to 6-membered saturated or partially unsaturated monocyclic heterocyclyl having 1-3 heteroatoms independently selected from nitrogen, oxygen, and sulfur. In some embodiments, Ring D is optionally substituted 6-membered saturated or partially unsaturated monocyclic heterocyclyl having 1-3 heteroatoms independently selected from nitrogen, oxygen, and sulfur. In some embodiments, Ring D is optionally substituted 6-membered saturated or partially unsaturated monocyclic heterocyclyl having 1-2 heteroatoms independently selected from nitrogen, oxygen, and sulfur.

In some embodiments, Ring D is optionally substituted 5- to 6-membered monocyclic heteroaryl having 1-4 heteroatoms independently selected from nitrogen, oxygen, and sulfur. In some embodiments, Ring D is optionally substituted 5-membered monocyclic heteroaryl having 1-4 heteroatoms independently selected from nitrogen, oxygen, and sulfur. In some embodiments, Ring D is optionally substituted 5-membered monocyclic heteroaryl having 1-2 heteroatoms independently selected from nitrogen, oxygen, and sulfur.

In some embodiments, each R is independently hydrogen or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or two R when attached to the same nitrogen atom are taken together to form optionally substituted 3- to 7-membered saturated or partially unsaturated monocyclic heterocyclyl having 0-2 additional heteroatoms independently selected from nitrogen, oxygen, and sulfur.

In some embodiments, each R is independently selected from hydrogen, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected RN substituents; or
two R when attached to the same nitrogen atom are taken together to form a 3- to 7-membered saturated or partially unsaturated monocyclic heterocyclyl having 0-2 additional heteroatoms independently selected from nitrogen, oxygen, and sulfur, wherein the 3- to 7-membered saturated or partially unsaturated monocyclic heterocyclyl is optionally substituted with 1, 2, 3, or 4 independently selected RN substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O) OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘}₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, each R is independently hydrogen or optionally substituted C₁₋₆ aliphatic. In some embodiments, R is hydrogen. In some embodiments, R is optionally substituted C₁₋₆ aliphatic. In some embodiments, R is optionally substituted C₁₋₃ aliphatic. In some embodiments, R is optionally substituted C₁₋₂ aliphatic. In some embodiments, R is optionally substituted C₁ aliphatic. In some embodiments, R is methyl.

In some embodiments, each R′ is independently an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or two R′ when attached to the same nitrogen atom are taken together to form optionally substituted 3- to 7-membered saturated or partially unsaturated monocyclic heterocyclyl having 0-2 additional heteroatoms independently selected from nitrogen, oxygen, and sulfur.

In some embodiments, each R′ is independently selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected RN substituents; or
two R′ when attached to the same nitrogen atom are taken together to a 3- to 7-membered saturated or partially unsaturated monocyclic heterocyclyl having 0-2 additional heteroatoms independently selected from nitrogen, oxygen, and sulfur, wherein the 3- to 7-membered saturated or partially unsaturated monocyclic heterocyclyl is optionally substituted with 1, 2, 3, or 4 independently selected RN substituents; and
each R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂.

In some embodiments, each R^{m} is independently —OH, —CN, or R.

In some embodiments, m is 1, 2, 3, or 4. In some embodiments, m is 0 or 1. In some embodiments, m is 0. In some embodiments, m is 1.

In some embodiments, n is 1, 2, 3, or 4. In some embodiments, n is 0 or 2. In some embodiments, n is 0. In some embodiments, n is 1. In some embodiments, n is 2.

In some embodiments, p is 1, 2, 3, or 4. In some embodiments, p is 1 or 2. In some embodiments, p is 1. In some embodiments, p is 2.

In some embodiments of any of Formulae I, VIII, VIII-a, VIII-b, VIII-c, VIII-d, IX, IX-a, IX-b, IX-c, and IX-d:
X is —N(R^{a})—;
R^{a} is -L^{R3}-R³;
L^{R3} is a covalent bond;
R³ is hydrogen or optionally substituted C₁₋₆ aliphatic;
Ring E is selected from phenyl and 5- to 6-membered monocyclic heteroaryl having 1-4 heteroatoms independently selected from nitrogen, oxygen, and sulfur;
each R^{4A} is independently selected from C₁₋₆ aliphatic and —OC₁₋₆ aliphatic;
R⁶ is hydrogen, deuterium, or optionally substituted C₁₋₆ aliphatic;
R⁷ is hydrogen, deuterium, or optionally substituted C₁₋₆ aliphatic;
D² is CH;
D³ is CR¹⁰;
R¹⁰ is hydrogen or halogen;
Ring B is 3- to 7-membered saturated or partially unsaturated monocyclic heterocyclylene having 1-3 heteroatoms independently selected from nitrogen, oxygen, and sulfur;
Ring C is 5- to 6-membered monocyclic heteroaryl having 1-4 heteroatoms independently selected from nitrogen, oxygen, and sulfur;
each L^{RC} is a covalent bond;
each R¹² is independently selected from halogen, C₁₋₆ aliphatic, —C(O)N(R)₂, and —C(O)NR(OR);
each R is independently selected from hydrogen, C₁₋₆ aliphatic, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur;
n is 0 or 2;
p is 1, 2, or 3; and
q is 0, 1, or 2.

In some embodiments of any of Formulae I, VIII, VIII-a, VIII-b, VIII-c, VIII-d, IX, IX-a, IX-b, IX-c, and IX-d:
X is —N(R^{a})—;
R^{a} is -L^{R3}-R³,
L^{R3} is a covalent bond;
R³ is hydrogen or optionally substituted C₁₋₆ aliphatic;
Ring E is a pyrimidine, pyrimidinone, pyridazine, or pyridazinone ring;
each R^{4A} is independently selected from C₁₋₆ aliphatic and —OC₁₋₆ aliphatic;
R⁶ is hydrogen, deuterium, or optionally substituted C₁₋₆ aliphatic;
R⁷ is hydrogen, deuterium, or optionally substituted C₁₋₆ aliphatic;
D² is CH;
D³ is CR¹⁰,
R¹⁰ is hydrogen or halogen;
Ring B is

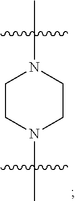 [SMILES: *N1CCN(*)CC1]

Ring C is phenyl or 5- to 6-membered monocyclic heteroaryl having 1-4 heteroatoms independently selected from nitrogen, oxygen, and sulfur;
each L^{RC} is a covalent bond;
each R¹² is independently selected from halogen, C₁₋₆ aliphatic, —C(O)N(R)₂, and —C(O)NR(OR);
each R is independently selected from hydrogen, C₁₋₆ aliphatic, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur;
n is 0;
p is 1, 2, or 3; and
q is 0, 1, or 2.

In some embodiments, the present disclosure provides compounds selected from Table 1:

TABLE 1

| | |
|---|---|
| 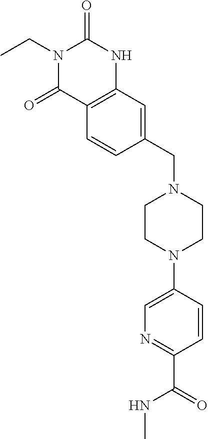 [SMILES: CCn1c(=O)[nH]c2cc(CN3CCN(c4ccc(C(=O)NC)nc4)CC3)ccc2c1=O] | I-1 |
| 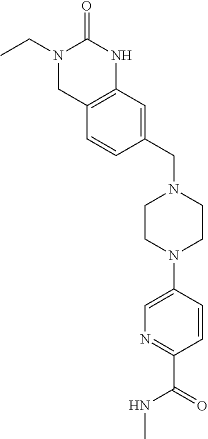 [SMILES: CCN1Cc2ccc(CN3CCN(c4ccc(C(=O)NC)nc4)CC3)cc2NC1=O] | I-2 |
| 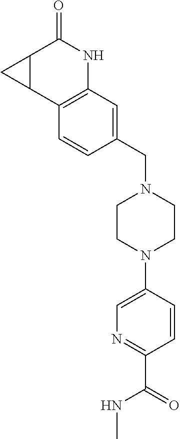 [SMILES: CNC(=O)c1ccc(N2CCN(Cc3ccc4c(c3)NC(=O)C3CC43)CC2)cn1] | I-3 |
| 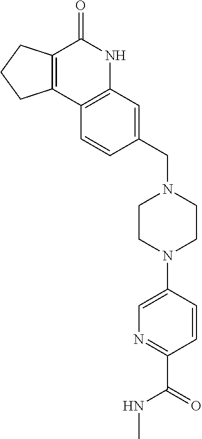 [SMILES: CNC(=O)c1ccc(N2CCN(Cc3ccc4c5c(c(=O)[nH]c4c3)CCC5)CC2)cn1] | I-4 |
| 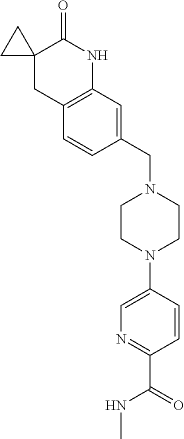 [SMILES: CNC(=O)c1ccc(N2CCN(Cc3ccc4c(c3)NC(=O)C3(CC3)C4)CC2)cn1] | I-5 |
| 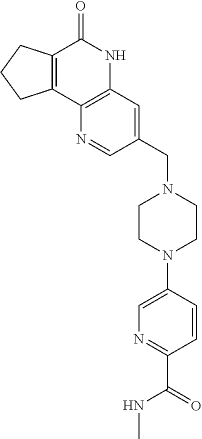 [SMILES: CNC(=O)c1ccc(N2CCN(Cc3cnc4c5c(c(=O)[nH]c4c3)CCC5)CC2)cn1] | I-6 |
| 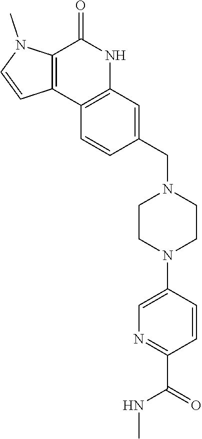 [SMILES: CNC(=O)c1ccc(N2CCN(Cc3ccc4c(c3)[nH]c(=O)c3c4ccn3C)CC2)cn1] | I-7 |
| 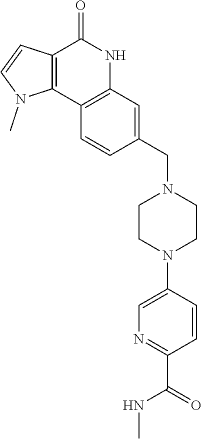 [SMILES: CNC(=O)c1ccc(N2CCN(Cc3ccc4c(c3)[nH]c(=O)c3ccn(C)c34)CC2)cn1] | I-8 |
| 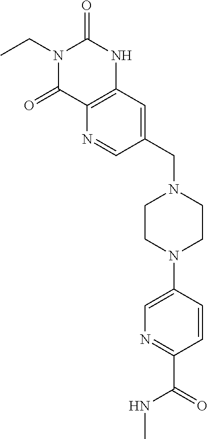 [SMILES: CCn1c(=O)[nH]c2cc(CN3CCN(c4ccc(C(=O)NC)nc4)CC3)cnc2c1=O] | I-9 |
| 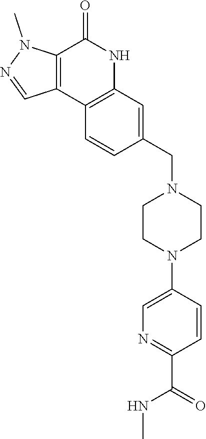 [SMILES: CNC(=O)c1ccc(N2CCN(Cc3ccc4c(c3)[nH]c(=O)c3c4cnn3C)CC2)cn1] | I-10 |
| 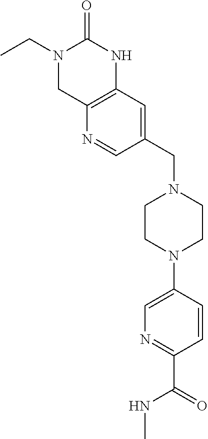 [SMILES: CCN1Cc2ncc(CN3CCN(c4ccc(C(=O)NC)nc4)CC3)cc2NC1=O] | I-11 |
| 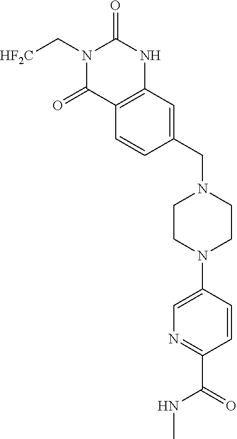 | I-12 |
| 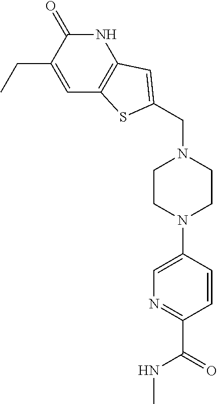 [SMILES: CCc1cc2sc(CN3CCN(c4ccc(C(=O)NC)nc4)CC3)cc2[nH]c1=O] | I-13 |
| 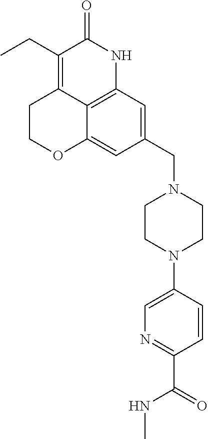 [SMILES: CCc1c2c3c(cc(CN4CCN(c5ccc(C(=O)NC)nc5)CC4)cc3[nH]c1=O)OCC2] | I-14 |
| 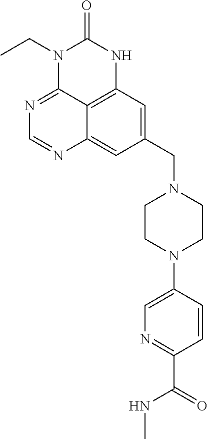 [SMILES: CCN1C(=O)Nc2cc(CN3CCN(c4ccc(C(=O)NC)nc4)CC3)cc3ncnc1c23] | I-15 |
| 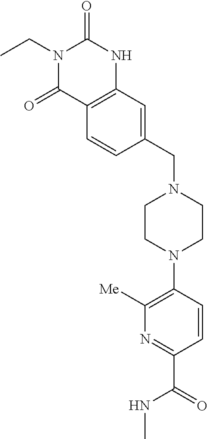 [SMILES: CCn1c(=O)[nH]c2cc(CN3CCN(c4ccc(C(=O)NC)nc4C)CC3)ccc2c1=O] | I-16 |
| 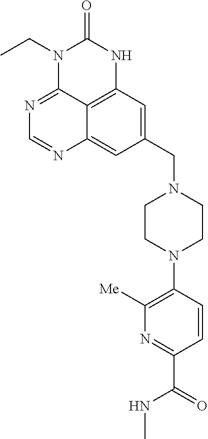 [SMILES: CCN1C(=O)Nc2cc(CN3CCN(c4ccc(C(=O)NC)nc4C)CC3)cc3ncnc1c23] | I-17 |
| 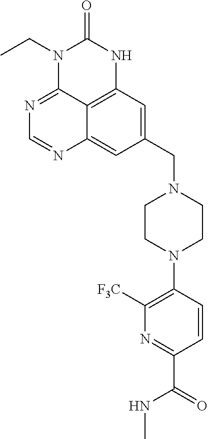 [SMILES: CCN1C(=O)Nc2cc(CN3CCN(c4ccc(C(=O)NC)nc4C(F)(F)F)CC3)cc3ncnc1c23] | I-18 |
| 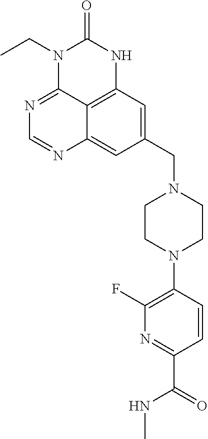 [SMILES: CCN1C(=O)Nc2cc(CN3CCN(c4ccc(C(=O)NC)nc4F)CC3)cc3ncnc1c23] | I-19 |
| 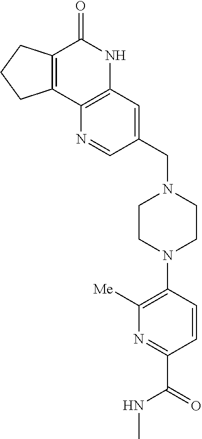 [SMILES: CNC(=O)c1ccc(N2CCN(Cc3cnc4c5c(c(=O)[nH]c4c3)CCC5)CC2)c(C)n1] | I-20 |
| 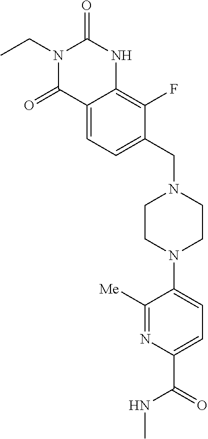 [SMILES: CCn1c(=O)[nH]c2c(F)c(CN3CCN(c4ccc(C(=O)NC)nc4C)CC3)ccc2c1=O] | I-21 |
| 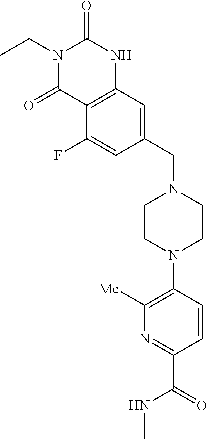 [SMILES: CCn1c(=O)[nH]c2cc(CN3CCN(c4ccc(C(=O)NC)nc4C)CC3)cc(F)c2c1=O] | I-22 |
| 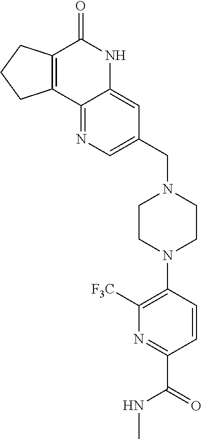 [SMILES: CNC(=O)c1ccc(N2CCN(Cc3cnc4c5c(c(=O)[nH]c4c3)CCC5)CC2)c(C(F)(F)F)n1] | I-23 |
| 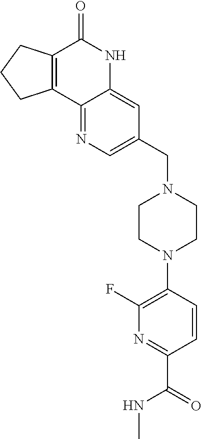 [SMILES: CNC(=O)c1ccc(N2CCN(Cc3cnc4c5c(c(=O)[nH]c4c3)CCC5)CC2)c(F)n1] | I-24 |
| 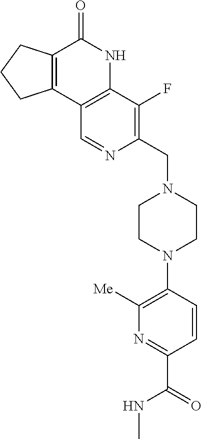 [SMILES: CNC(=O)c1ccc(N2CCN(Cc3ncc4c5c(c(=O)[nH]c4c3F)CCC5)CC2)c(C)n1] | I-25 |
| 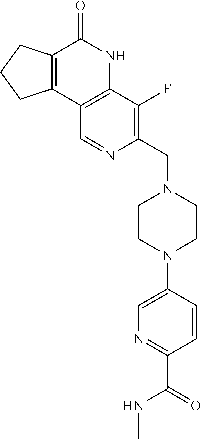 [SMILES: CNC(=O)c1ccc(N2CCN(Cc3ncc4c5c(c(=O)[nH]c4c3F)CCC5)CC2)cn1] | I-26 |
| 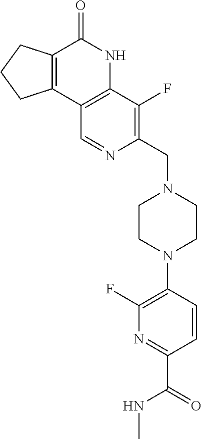 [SMILES: CNC(=O)c1ccc(N2CCN(Cc3ncc4c5c(c(=O)[nH]c4c3F)CCC5)CC2)c(F)n1] | I-27 |
| 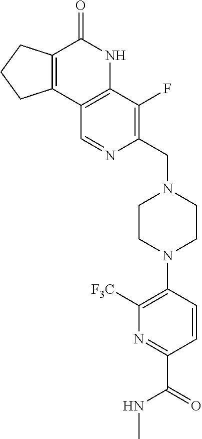 [SMILES: CNC(=O)c1ccc(N2CCN(Cc3ncc4c5c(c(=O)[nH]c4c3F)CCC5)CC2)c(C(F)(F)F)n1] | I-28 |
| 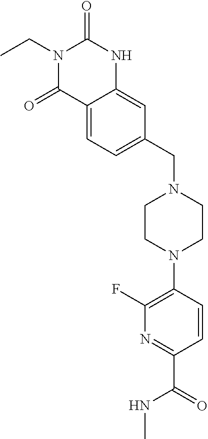 [SMILES: CCn1c(=O)[nH]c2cc(CN3CCN(c4ccc(C(=O)NC)nc4F)CC3)ccc2c1=O] | I-29 |
| 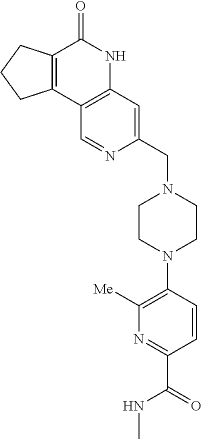 [SMILES: CNC(=O)c1ccc(N2CCN(Cc3cc4[nH]c(=O)c5c(c4cn3)CCC5)CC2)c(C)n1] | I-30 |
| 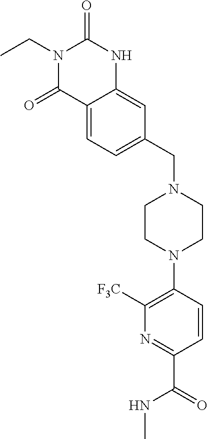 [SMILES: CCn1c(=O)[nH]c2cc(CN3CCN(c4ccc(C(=O)NC)nc4C(F)(F)F)CC3)ccc2c1=O] | I-31 |
| 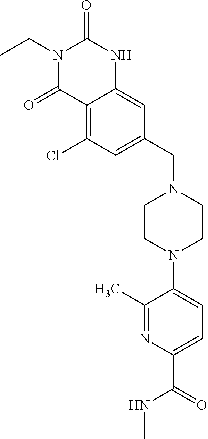 [SMILES: CCn1c(=O)[nH]c2cc(CN3CCN(c4ccc(C(=O)NC)nc4C)CC3)cc(Cl)c2c1=O] | I-32 |
| 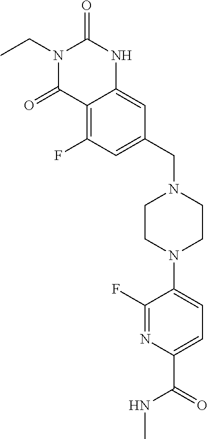 [SMILES: CCn1c(=O)[nH]c2cc(CN3CCN(c4ccc(C(=O)NC)nc4F)CC3)cc(F)c2c1=O] | I-33 |

or a pharmaceutically acceptable salt thereof.

In some embodiments, the compound provided herein is selected from:
5-(4-((3-ethyl-2,4-dioxo-1,2,3,4-tetrahydroquinazolin-7-yl)methyl)piperazin-1-yl)-N-methylpicolinamide;
5-(4-((3-ethyl-2-oxo-1,2,3,4-tetrahydroquinazolin-7-yl)methyl)piperazin-1-yl)-N-methylpicolinamide;
N-methyl-5-(4-((2-oxo-1a,2,3,7b-tetrahydro-1H-cyclopropa[c]quinolin-5-yl)methyl)piperazin-1-yl)picolinamide;
N-methyl-5-(4-((4-oxo-2,3,4,5-tetrahydro-1H-cyclopenta[c]quinolin-7-yl)methyl)piperazin-1-yl)picolinamide;
N-methyl-5-(4-((2′-oxo-1′,4′-dihydro-2′H-spiro[cyclopropane-1,3′-quinolin]-7′-yl)methyl)piperazin-1-yl)picolinamide;
N-methyl-5-(4-((6-oxo-6,7,8,9-tetrahydro-5H-cyclopenta[c][1,5]naphthyridin-3-yl)methyl)piperazin-1-yl)picolinamide;
N-methyl-5-(4-((3-methyl-4-oxo-4,5-dihydro-3H-pyrrolo[2,3-c]quinolin-7-yl)methyl)piperazin-1-yl)picolinamide;
N-methyl-5-(4-((1-methyl-4-oxo-4,5-dihydro-1H-pyrrolo[3,2-c]quinolin-7-yl)methyl)piperazin-1-yl)picolinamide;
5-(4-((3-ethyl-2,4-dioxo-1,2,3,4-tetrahydropyrido[3,2-d]pyrimidin-7-yl)methyl)piperazin-1-yl)-N-methylpicolinamide;
N-methyl-5-(4-((3-methyl-4-oxo-4,5-dihydro-3H-pyrazolo[3,4-c]quinolin-7-yl)methyl)piperazin-1-yl)picolinamide;
5-(4-((3-ethyl-2-oxo-1,2,3,4-tetrahydropyrido[3,2-d]pyrimidin-7-yl)methyl)piperazin-1-yl)-N-methylpicolinamide;
5-(4-((3-(2,2-difluoroethyl)-2,4-dioxo-1,2,3,4-tetrahydroquinazolin-7-yl)methyl)piperazin-1-yl)-N-methylpicolinamide;
5-(4-((6-ethyl-5-oxo-4,5-dihydrothieno[3,2-b]pyridin-2-yl)methyl)piperazin-1-yl)-N-methylpicolinamide;
5-(4-((4-ethyl-5-oxo-2,3,5,6-tetrahydropyrano[4,3,2-de]quinolin-8-yl)methyl)piperazin-1-yl)-N-methylpicolinamide;
5-(4-((3-ethyl-2-oxo-2,3-dihydro-1H-pyrimido[4,5,6-de]quinazolin-8-yl)methyl)piperazin-1-yl)-N-methylpicolinamide;
5-(4-((3-ethyl-2,4-dioxo-1,2,3,4-tetrahydroquinazolin-7-yl)methyl)piperazin-1-yl)-N,6-dimethylpicolinamide;
5-(4-((3-ethyl-2-oxo-2,3-dihydro-1H-pyrimido[4,5,6-de]quinazolin-8-yl)methyl)piperazin-1-yl)-N,6-dimethylpicolinamide;
5-(4-((3-ethyl-2-oxo-2,3-dihydro-1H-pyrimido[4,5,6-de]quinazolin-8-yl)methyl)piperazin-1-yl)-N-methyl-6-(trifluoromethyl)picolinamide;
5-(4-((3-ethyl-2-oxo-2,3-dihydro-1H-pyrimido[4,5,6-de]quinazolin-8-yl)methyl)piperazin-1-yl)-6-fluoro-N-methylpicolinamide;
N,6-dimethyl-5-(4-((6-oxo-6,7,8,9-tetrahydro-5H-cyclopenta[c][1,5]naphthyridin-3-yl)methyl)piperazin-1-yl)picolinamide;
5-(4-((3-ethyl-8-fluoro-2,4-dioxo-1,2,3,4-tetrahydroquinazolin-7-yl)methyl)piperazin-1-yl)-N,6-dimethylpicolinamide;
5-(4-((3-ethyl-5-fluoro-2,4-dioxo-1,2,3,4-tetrahydroquinazolin-7-yl)methyl)piperazin-1-yl)-N,6-dimethylpicolinamide;
N-methyl-5-(4-((6-oxo-6,7,8,9-tetrahydro-5H-cyclopenta[c][1,5]naphthyridin-3-yl)methyl)piperazin-1-yl)-6-(trifluoromethyl)picolinamide;
6-fluoro-N-methyl-5-(4-((6-oxo-6,7,8,9-tetrahydro-5H-cyclopenta[c][1,5]naphthyridin-3-yl)methyl)piperazin-1-yl)picolinamide;
5-(4-((4-fluoro-6-oxo-6,7,8,9-tetrahydro-5H-cyclopenta[c][1,6]naphthyridin-3-yl)methyl)piperazin-1-yl)-N,6-dimethylpicolinamide;
5-(4-((4-fluoro-6-oxo-6,7,8,9-tetrahydro-5H-cyclopenta[c][1,6]naphthyridin-3-yl)methyl)piperazin-1-yl)-N-methylpicolinamide;
6-fluoro-5-(4-((4-fluoro-6-oxo-6,7,8,9-tetrahydro-5H-cyclopenta[c][1,6]naphthyridin-3-yl)methyl)piperazin-1-yl)-N-methylpicolinamide;
5-(4-((4-fluoro-6-oxo-6,7,8,9-tetrahydro-5H-cyclopenta[c][1,6]naphthyridin-3-yl)methyl)piperazin-1-yl)-N-methyl-6-(trifluoromethyl)picolinamide;
5-(4-((3-ethyl-2,4-dioxo-1,2,3,4-tetrahydroquinazolin-7-yl)methyl)piperazin-1-yl)-6-fluoro-N-methylpicolinamide;
N,6-dimethyl-5-(4-((6-oxo-6,7,8,9-tetrahydro-5H-cyclopenta[c][1,6]naphthyridin-3-yl)methyl)piperazin-1-yl)picolinamide;
5-(4-((3-ethyl-2,4-dioxo-1,2,3,4-tetrahydroquinazolin-7-yl)methyl)piperazin-1-yl)-N-methyl-6-(trifluoromethyl)picolinamide;
5-(4-((5-chloro-3-ethyl-2,4-dioxo-1,2,3,4-tetrahydroquinazolin-7-yl)methyl)piperazin-1-yl)-N,6-dimethylpicolinamide;
5-(4-((3-ethyl-5-fluoro-2,4-dioxo-1,2,3,4-tetrahydroquinazolin-7-yl)methyl)piperazin-1-yl)-6-fluoro-N-methylpicolinamide;
5-(4-((3-ethyl-6-fluoro-1-methyl-4-oxo-1,3,4,5-tetrahydropyrazolo[3,4,5-de]quinazolin-7-yl)methyl)piperazin-1-yl)-N,6-dimethylpicolinamide;
5-(4-((3-ethyl-9-fluoro-2-oxo-2,3-dihydro-1H-pyrimido[4,5,6-de]quinazolin-8-yl)methyl)piperazin-1-yl)-N,6-dimethylpicolinamide;
5-(4-((3-ethyl-5-fluoro-2,4-dioxo-1,2,3,4-tetrahydroquinazolin-7-yl)methyl)piperazin-1-yl)-N,6-bis(methyl-d3)picolinamide;
5-(4-((5-(difluoromethyl)-3-ethyl-2,4-dioxo-1,2,3,4-tetrahydroquinazolin-7-yl)methyl)piperazin-1-yl)-N,6-dimethylpicolinamide;
5-(4-((8-fluoro-5-methoxy-3-methyl-2,4-dioxo-1,2,3,4-tetrahydroquinazolin-7-yl)methyl)piperazin-1-yl)-N,6-dimethylpicolinamide;
5-(4-((5-chloro-3-ethyl-8-fluoro-2,4-dioxo-1,2,3,4-tetrahydroquinazolin-7-yl)methyl)piperazin-1-yl)-N,6-dimethylpicolinamide;
5-(4-((5-cyclopropyl-3-ethyl-8-fluoro-2,4-dioxo-1,2,3,4-tetrahydroquinazolin-7-yl)methyl)piperazin-1-yl)-N,6-dimethylpicolinamide;
5-(4-((3-ethyl-8-fluoro-5-(hydroxymethyl)-2,4-dioxo-1,2,3,4-tetrahydroquinazolin-7-yl)methyl)piperazin-1-yl)-N,6-dimethylpicolinamide;
5-(4-((5-(cyanomethyl)-3-ethyl-8-fluoro-2,4-dioxo-1,2,3,4-tetrahydroquinazolin-7-yl)methyl)piperazin-1-yl)-N,6-dimethylpicolinamide;
5-(4-((5-chloro-3-methyl-2,4-dioxo-1,2,3,4-tetrahydroquinazolin-7-yl)methyl)piperazin-1-yl)-N-ethyl-6-methylpicolinamide;
5-(4-((3-ethyl-2,4-dioxo-1,2,3,4-tetrahydroquinazolin-7-yl)methyl)piperazin-1-yl)-N,6-bis(methyl-d3)picolinamide;
5-[4-[(12-ethyl-11-oxo-2,3,10,12-tetrazatricyclo[7.3.1.05,13]trideca-1,3,5,7,9 (13)-pentaen-7-yl)methyl]piperazin-1-yl]-N,6-dimethyl-pyridine-2-carboxamide;
5-(4-((3-ethyl-8-fluoro-5-(methoxymethyl)-2,4-dioxo-1,2,3,4-tetrahydroquinazolin-7-yl)methyl)piperazin-1-yl)-N,6-dimethylpicolinamide;
5-(4-((5-(difluoromethyl)-3-methyl-2,4-dioxo-1,2,3,4-tetrahydroquinazolin-7-yl)methyl)piperazin-1-yl)-N,6-dimethylpicolinamide;
5-(4-((5-chloro-3-ethyl-2,4-dioxo-1,2,3,4-tetrahydroquinazolin-7-yl)methyl)piperazin-1-yl)-N-ethyl-6-methylpicolinamide;
5-(4-((5-cyclopropyl-8-fluoro-3-methyl-2,4-dioxo-1,2,3,4-tetrahydroquinazolin-7-yl)methyl)piperazin-1-yl)-N,6-dimethylpicolinamide;
5-(4-((3-ethyl-9-fluoro-5-methoxy-2-oxo-2,3-dihydro-1H-pyrimido[4,5,6-de]quinazolin-8-yl)methyl)piperazin-1-yl)-N,6-dimethylpicolinamide;
5-(4-((3-ethyl-9-fluoro-6-methyl-2,5-dioxo-2,3,5,6-tetrahydro-1H-pyrimido[4,5,6-de]quinazolin-8-yl)methyl)piperazin-1-yl)-N,6-dimethylpicolinamide;
5-(4-((3-ethyl-9-fluoro-5-methyl-2-oxo-2,3-dihydro-1H-pyrimido[4,5,6-de]quinazolin-8-yl)methyl)piperazin-1-yl)-N,6-dimethylpicolinamide;
5-(4-((3-ethyl-9-fluoro-2-oxo-2,3-dihydro-1H-pyrimido[4,5,6-de]quinazolin-8-yl)methyl-d2)piperazin-1-yl)-N,6-dimethylpicolinamide;
5-(4-((9-ethyl-6-fluoro-3-methyl-8-oxo-8,9-dihydro-7H-pyridazino[3,4,5-de]quinazolin-5-yl)methyl)piperazin-1-yl)-N,6-dimethylpicolinamide;
N-cyclopropyl-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-cyclopropyl-5-(4-((9-ethyl-6-fluoro-3-methyl-8-oxo-8,9-dihydro-7H-pyridazino[3,4,5-de]quinazolin-5-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-N-methoxy-6-methylpicolinamide;
N-(cyclopropylmethoxy)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-N-(1-(hydroxymethyl)cyclopropyl)-6-methylpicolinamide;
N-((1r,3r)-3-cyanocyclobutyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydro-2H-pyran-4-yl)picolinamide;
N-(1-cyanocyclopropyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
(S)-5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydro-2H-pyran-3-yl)picolinamide;
N-(3,3-difluorocyclobutyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-((1s,3s)-3-cyanocyclobutyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-N-(1-(methoxymethyl)cyclopropyl)-6-methylpicolinamide;
(R)-5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydrofuran-3-yl)picolinamide;
(R)-5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydro-2H-pyran-3-yl)picolinamide;
N-((1R,5S,6s)-3-oxabicyclo[3.1.0]hexan-6-yl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-((1R,5S,6r)-3-oxabicyclo[3.1.0]hexan-6-yl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-6-methyl-N-(1-methylazetidin-3-yl)picolinamide;
5-(4-((3-ethyl-9-fluoro-2-oxo-2,3-dihydro-1H-pyrimido[4,5,6-de]quinazolin-8-yl)methyl)piperazin-1-yl)-N-((1s,3s)-3-hydroxycyclobutyl)-6-methylpicolinamide;
(S)-5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydrofuran-3-yl)picolinamide;
N-((1s,4s)-4-cyanocyclohexyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-((1r,4r)-4-cyanocyclohexyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-6-methyl-N-(1-methylpiperidin-4-yl)picolinamide;
(R)-5-(4-((3-ethyl-9-fluoro-2-oxo-2,3-dihydro-1H-pyrimido[4,5,6-de]quinazolin-8-yl)methyl)piperazin-1-yl)-6-methyl-N-(1-methylpyrrolidin-3-yl)picolinamide;
(S)-5-(4-((3-ethyl-9-fluoro-2-oxo-2,3-dihydro-1H-pyrimido[4,5,6-de]quinazolin-8-yl)methyl)piperazin-1-yl)-6-methyl-N-(1-methylpyrrolidin-3-yl)picolinamide;
N-(1-(cyanomethyl)cyclopropyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-cyclopropyl-5-(4-((9-ethyl-6-fluoro-2-methyl-3,8-dioxo-2,7,8,9-tetrahydro-3H-pyridazino[3,4,5-de]quinazolin-5-yl)methyl)piperazin-1-yl)-6-methylpicolinamide;
4-[4-[(6-ethyl-10-fluoro-7-oxo-2,4,6,8-tetrazatricyclo[7.3.1.05,13]trideca-1,3,5 (13),9,11-pentaen-11-yl)methyl]piperazin-1-yl]-N,3-dimethyl-benzamide;
N-cyclopropyl-4-(4-((3-ethyl-9-fluoro-2-oxo-2,3-dihydro-1H-pyrimido[4,5,6-de]quinazolin-8-yl)methyl)piperazin-1-yl)-3-methylbenzamide;
4-(4-((3-ethyl-9-fluoro-2-oxo-2,3-dihydro-1H-pyrimido[4,5,6-de]quinazolin-8-yl)methyl)piperazin-1-yl)-N-methoxy-3-methylbenzamide;
N-(cyclopropylmethoxy)-4-(4-((3-ethyl-9-fluoro-2-oxo-2,3-dihydro-1H-pyrimido[4,5,6-de]quinazolin-8-yl)methyl)piperazin-1-yl)-3-methylbenzamide;
4-(4-((3-ethyl-9-fluoro-2-oxo-2,3-dihydro-1H-pyrimido[4,5,6-de]quinazolin-8-yl)methyl)piperazin-1-yl)-3-fluoro-N-methylbenzamide;
N-cyclopropyl-4-(4-((3-ethyl-9-fluoro-2-oxo-2,3-dihydro-1H-pyrimido[4,5,6-de]quinazolin-8-yl)methyl)piperazin-1-yl)-3-fluorobenzamide;
4-(4-((3-ethyl-9-fluoro-2-oxo-2,3-dihydro-1H-pyrimido[4,5,6-de]quinazolin-8-yl)methyl)piperazin-1-yl)-3-fluoro-N-methoxybenzamide;
N-(cyclopropylmethoxy)-4-(4-((3-ethyl-9-fluoro-2-oxo-2,3-dihydro-1H-pyrimido[4,5,6-de]quinazolin-8-yl)methyl)piperazin-1-yl)-3-fluorobenzamide;
4-(4-((3-ethyl-9-fluoro-2-oxo-2,3-dihydro-1H-pyrimido[4,5,6-de]quinazolin-8-yl)methyl)piperazin-1-yl)-2,3-difluoro-N-methylbenzamide;
N-cyclopropyl-4-(4-((3-ethyl-9-fluoro-2-oxo-2,3-dihydro-1H-pyrimido[4,5,6-de]quinazolin-8-yl)methyl)piperazin-1-yl)-2,3-difluorobenzamide;
4-(4-((3-ethyl-9-fluoro-2-oxo-2,3-dihydro-1H-pyrimido[4,5,6-de]quinazolin-8-yl)methyl)piperazin-1-yl)-2,3-difluoro-N-methoxybenzamide;
N-(cyclopropylmethoxy)-4-(4-((3-ethyl-9-fluoro-2-oxo-2,3-dihydro-1H-pyrimido[4,5,6-de]quinazolin-8-yl)methyl)piperazin-1-yl)-2,3-difluorobenzamide;
N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-3-methylbenzamide;
N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-3-fluorobenzamide;
N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-2,3-difluorobenzamide; and
N-cyclobutyl-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
   or a pharmaceutically acceptable salt thereof.

In some embodiments, the compound provided herein is 5-(4-((3-ethyl-9-fluoro-2-oxo-2,3-dihydro-1H-pyrimido[4,5,6-de]quinazolin-8-yl)methyl)piperazin-1-yl)-N,6-dimethylpicolinamide, or a pharmaceutically acceptable salt thereof.

In some embodiments, the compound provided herein is N-cyclopropyl-5-(4-((3-ethyl-9-fluoro-2-oxo-2,3-dihydro-1H-pyrimido[4,5,6-de]quinazolin-8-yl)methyl)piperazin-1-yl)-6-methylpicolinamide, or a pharmaceutically acceptable salt thereof.

In some embodiments, the present disclosure encompasses the recognition that provided compounds display certain desirable characteristics, e.g., as compared to other known compounds. For example, in some embodiments, provided compounds are more potent in one or more biochemical or cellular assays described herein, and/or have one or more other characteristics that make them more suitable for drug development, such as better selectivity for PARP1 over other PARP enzymes and/or better ADME (absorption, distribution, metabolism, and excretion) properties including but not limited to better permeability, cytotoxicity, hepatocyte stability, solubility, and/or plasma protein binding profiles, than other known compounds. In some embodiments, provided compounds display certain desirable characteristics in one or more assays described herein, e.g., compared to other known compounds.

In some embodiments, provided compounds are provided and/or utilized in a salt form (e.g., a pharmaceutically acceptable salt form). Reference to a compound provided herein is understood to include reference to salts thereof, unless otherwise indicated.

It will be understood that, unless otherwise specified or prohibited by the foregoing definition of any of Formulae I, II, II-a, II-a-i, III, IV, V, VI, VI-a, VI-b, VII, VIII, VIII-a, VIII-b, VIII-c, VIII-d, IX, IX-a, IX-b, IX-c, and IX-d, embodiments of variables, X, R¹, R², R^{a}, R⁴, R⁵, Ring A, Ring A′, L^{R3}, R³, R^{L}, R^{L1}, L, R^{A1}, R⁶, R⁷, D¹, L^{D1}, R⁸, D², L^{D2}, R⁹, D³, R¹⁰, Ring B, Ring C, R^{B}, L^{RB}, R¹¹, R^{C}, L^{RC}, R¹², Ring D, R, R′, R^{m}, m, n, p, RIA, R^{3A}, R^{4A}, R^{6A}, R^{8A}, R^{9A}, R^{11A}, R^{B1}, R^{D1} and R^{N} as defined above and described in classes and subclasses herein, apply to compounds of any of Formulae I, II, II-a, II-a-i, III, IV, V, VI, VI-a, VI-b, VII, VIII, VIII-a, VIII-b, VIII-c, VIII-d, IX, IX-a, IX-b, IX-c, and IX-d, both singly and in combination.

It will be appreciated that throughout the present disclosure, unless otherwise indicated, reference to a compound of Formula I is intended to also include any of Formulae I, II, II-a, II-a-i, III, IV, V, VI, VI-a, VI-b, VII, VIII, VIII-a, VIII-b, VIII-c, VIII-d, IX, IX-a, IX-b, IX-c, and IX-d, and compound species of such formulae disclosed herein.

### Preparing Provided Compounds

Provided compounds may generally be made by the processes described in the ensuing schemes and examples.

In some embodiments, provided intermediates (e.g., compounds of Formula Int) are prepared according to the following Scheme:

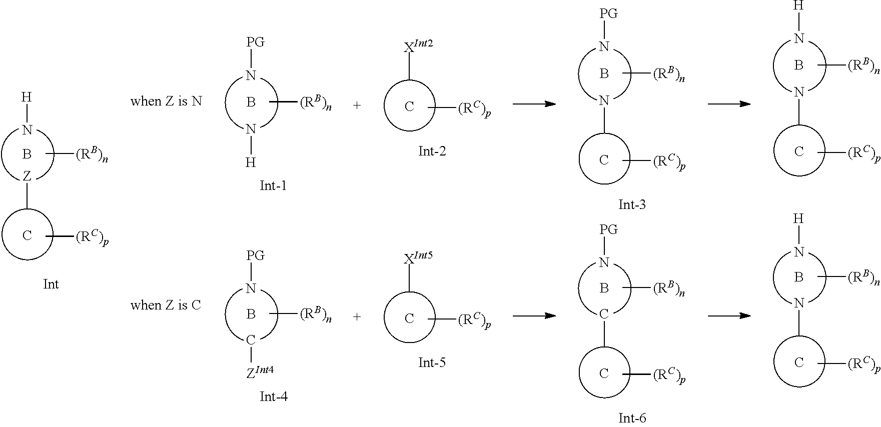

wherein Z is N or C, PG is a suitable protecting group (e.g., -Boc, -Cbz, or -SEM), X^{Int2} includes but is not limited to halogen, —B(OH)₂, and -OTf, each of Z^{Int4} and X^{Int5} includes but is not limited to halogen, -OTf, -Bpin, —Sn(Bu)₃, and —ZnBr, and each of Ring B, Ring C, R^{B}, R^{C}, n, and p is as defined above for Formula I, and described in classes and subclasses herein, both singly and in combination. Accordingly, in some embodiments, when Z is N, intermediate Int-3 is prepared by a process comprising contacting compounds of Formulae Int-1 and Int-2 under suitable conditions (e.g., nucleophilic aromatic substitution, Buchwald-Hartwig cross-coupling, Ullmann coupling, or Chan-Lam coupling). In some embodiments, when Z is C, intermediate Int-6 is prepared by a process comprising contacting compounds of Formulae Int-4 and Int-5 under suitable conditions (e.g., Suzuki, Stille, or Negishi coupling). In some embodiments, compounds of Formula Int are prepared by reacting intermediate Int-1-3 or Int-1-6 under suitable conditions (e.g., to remove PG).

In some embodiments, provided compounds are prepared according to the following Scheme:

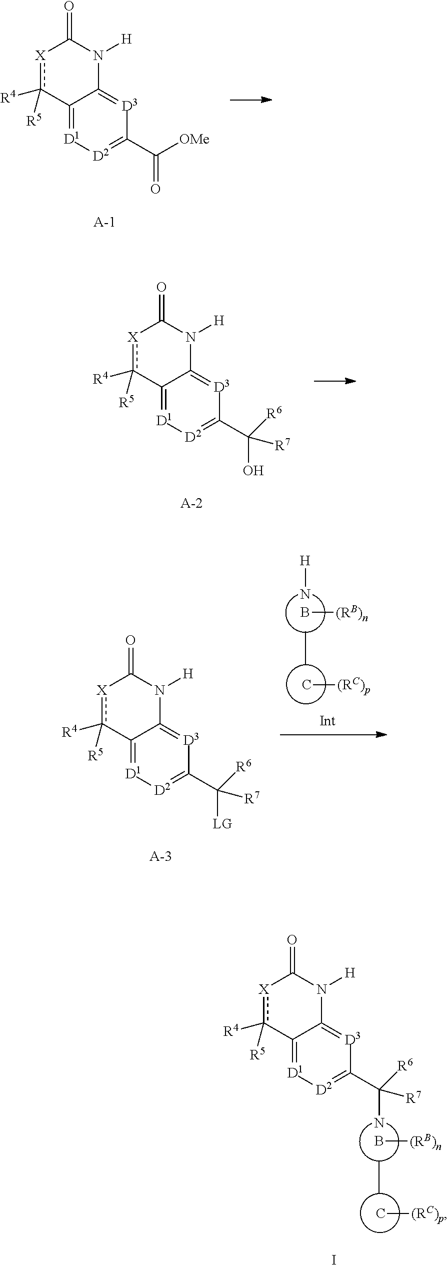

wherein LG is a suitable leaving group (e.g., halogen such as —Cl or —Br, or —OMs), and each of X, R⁴, R⁵, R⁶, R⁷, D¹, D², D³, Ring B, Ring C, R^{B}, R^{C}, n and p is as defined above for Formula I, and described in classes and subclasses herein, both singly and in combination. Accordingly, in some embodiments, intermediate A-2 is prepared by a process comprising contacting compounds of Formula A-1 with an appropriate reagent (e.g., a reducing agent such as LiAlH₄, DIBAL-H, and LiBHEt₃). In some embodiments, intermediate A-3 is prepared by a process comprising reacting intermediate A-2 under suitable conditions (e.g., HBr/AcOH, CBr₄/PPh₃, and MsCl/Et₃N). In some embodiments, compounds of Formula I are prepared by a process comprising contacting intermediates A-3 and Int under suitable conditions.

In some embodiments, provided compounds are prepared according to the following Scheme:

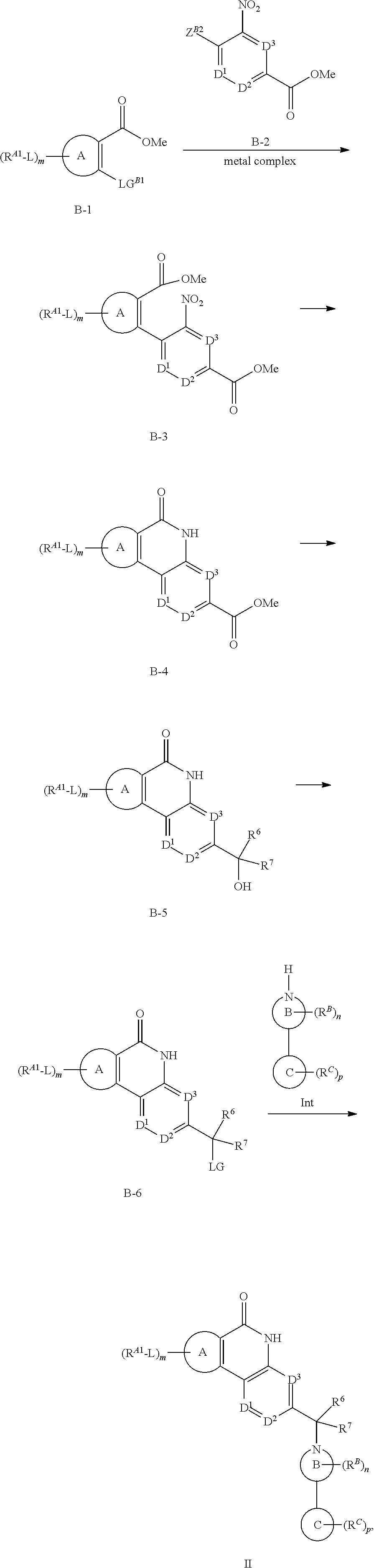

wherein LG^{B1} is a suitable leaving group (e.g., halogen such as —Cl or —Br, or —I, or —OTf), Z^{B2} is -Bpin or —Sn(Bu)₃, LG is a suitable leaving group (e.g., halogen such as —Cl or —Br, or —OMs), and each of R⁶, R⁷, D¹, D², D³, Ring A, Ring B, Ring C, R^{A1}, R^{B}, R^{C}, L, m, n, and p is as defined above for Formula II, and described in classes and subclasses herein, both singly and in combination. Accordingly, in some embodiments, intermediate B-3 is prepared by a process comprising contacting compounds of Formulae B-1 and B-2 in the presence of a suitable metal complex (e.g., a palladium precatalyst complex such as chloro(2-dicyclohexylphosphino-2′,4′,6′-triisoporpyl-1,1′-biphenyl)[2-(2′-amino-1,1′-biphenyl)]palladium(II)), and optionally in the presence of a suitable base (e.g., K₃PO₄, K₂CO₃, or Cs₂CO₃). In some embodiments, intermediate B-4 is prepared by a process comprising reacting compounds of Formula B-3 under suitable conditions (e.g., Fe/NH₄Cl). In some embodiments, intermediate B-5 is prepared by a process comprising contacting compounds of Formula B-4 with an appropriate reagent (e.g., a reducing agent such as LiAlH₄, DIBAL-H, and LiBHEt₃). In some embodiments, intermediate B-6 is prepared by a process comprising reacting compounds of Formula B-5 under suitable conditions (e.g., HBr/AcOH, CBr₄/PPh₃, and MsCl/Et₃N). In some embodiments, compounds of Formula II are prepared by a process comprising contacting intermediates B-6 and Int under suitable conditions.

In some embodiments, provided compounds are prepared according to the following Scheme:

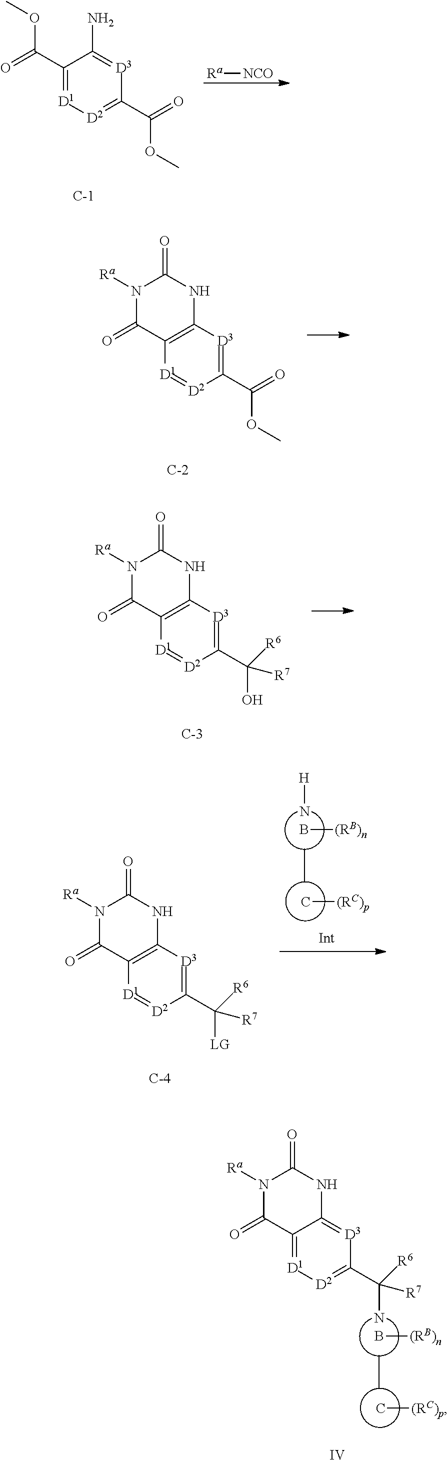

wherein LG is a suitable leaving group (e.g., halogen such as —Cl or —Br, or —OMs), and each of R^{a}, R⁶, R⁷, D¹, D′, D³, Ring B, Ring C, R^{B}, R^{C}, n, and p is as defined above for Formula IV, and described in classes and subclasses herein, both singly and in combination. Accordingly, in some embodiments, intermediate C-2 is prepared by a process comprising contacting compounds of Formula C-1 with an appropriate isocyanate of Formula R^{a}—NCO. In some embodiments, intermediate C-3 is prepared by a process comprising contacting compounds of Formula C-2 with an appropriate reagent (e.g., a reducing agent such as LiAlH₄, DIBAL-H, and LiBHEt₃). In some embodiments, intermediate C-4 is prepared by a process comprising reacting compounds of Formula C-3 under suitable conditions (e.g., HBr/AcOH, CBr₄/PPh₃, and MsCl/Et₃N). In some embodiments, compounds of Formula IV are prepared by a process comprising contacting intermediates C-4 and Int under suitable conditions.

In some embodiments, provided compounds are prepared according to the following Scheme:

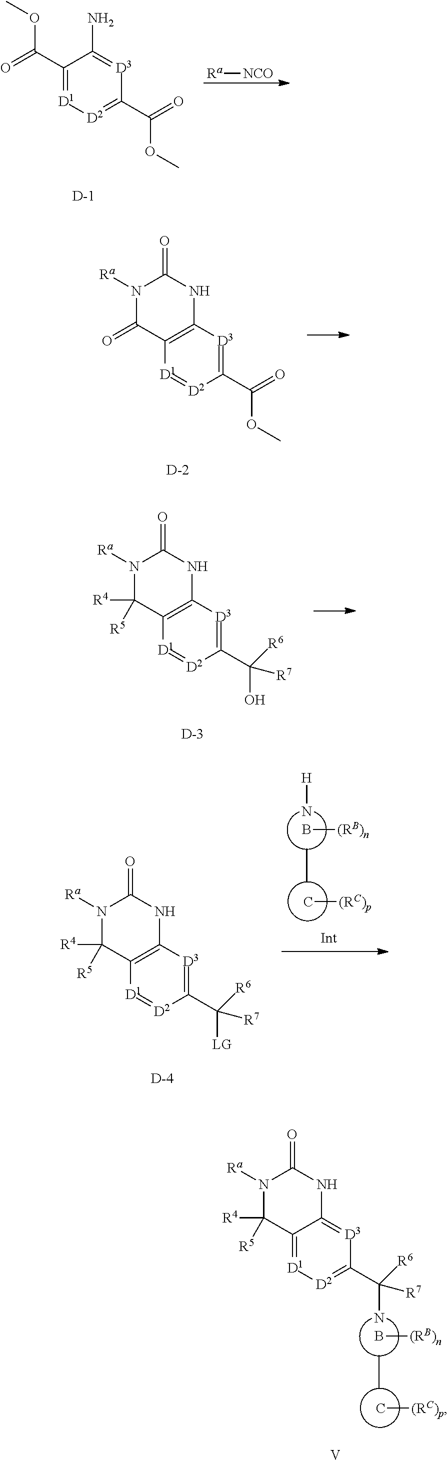

wherein LG is a suitable leaving group (e.g., halogen such as —Cl or —Br, or —OMs), and each of each of R^{a}, R⁴, R⁵, R⁶, R⁷, D¹, D², D³, Ring B, Ring C, R^{B}, R^{C}, n, and p is as defined above for Formula V, and described in classes and subclasses herein, both singly and in combination. Accordingly, in some embodiments, intermediate D-2 is prepared by a process comprising contacting compounds of Formula D-1 with an appropriate isocyanate of Formula R^{a}—NCO. In some embodiments, intermediate D-3 is prepared by a process comprising contacting compounds of Formula D-2 with an appropriate reagent (e.g., a reducing agent such as LiAlH₄, DIBAL-H, and LiBHEt₃). In some embodiments, intermediate D-4 is prepared by a process comprising reacting compounds of Formula D-3 under suitable conditions (e.g., HBr/AcOH, CBr₄/PPh₃, and MsCl/Et₃N). In some embodiments, compounds of Formula V are prepared by a process comprising contacting intermediates D-4 and Int under suitable conditions.

### Compositions

The present disclosure also provides compositions comprising a compound provided herein with one or more other components. In some embodiments, provided compositions comprise and/or deliver a compound described herein (e.g., compounds of any of Formulae I, II, II-a, II-a-i, III, IV, V, VI, VI-a, VI-b, VII, VIII, VIII-a, VIII-b, VIII-c, VIII-d, IX, IX-a, IX-b, IX-c, and IX-d).

In some embodiments, a provided composition is a pharmaceutical composition that comprises and/or delivers a compound provided herein (e.g., compounds of any of Formulae I, II, II-a, II-a-i, III, IV, V, VI, VI-a, VI-b, VII, VIII, VIII-a, VIII-b, VIII-c, VIII-d, IX, IX-a, IX-b, IX-c, and IX-d) and further comprises a pharmaceutically acceptable carrier.

Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injection forms, depot forms, as well as injectable and infusible solutions. Methods of preparing pharmaceutical compositions are well known in the art.

In some embodiments, provided compounds are formulated in a unit dosage form for ease of administration and uniformity of dosage. The expression “unit dosage form” as used herein refers to a physically discrete unit of an active agent (e.g., a compound described herein) for administration to a subject. Typically, each such unit contains a predetermined quantity of active agent. In some embodiments, a unit dosage form contains an entire single dose of the agent. In some embodiments, more than one unit dosage form is administered to achieve a total single dose. In some embodiments, administration of multiple unit dosage forms is required, or expected to be required, in order to achieve an intended effect. A unit dosage form may be, for example, a liquid pharmaceutical composition containing a predetermined quantity of one or more active agents, a solid pharmaceutical composition (e.g., a tablet, a capsule, or the like) containing a predetermined amount of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein.

### Uses

The present disclosure provides uses for compounds and compositions described herein. In some embodiments, provided compounds and compositions are for use in medicine (e.g., as therapy). In some embodiments, provided compounds and compositions are useful in treating a disease, disorder, or condition, wherein an underlying pathology is, wholly or partially, mediated by PARP1. In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with PARP1. In some embodiments, the present disclosure provides methods of administering provided compounds or compositions to a subject suffering from or susceptible to a disease, disorder, or condition, wherein an underlying pathology is, wholly or partially, mediated by PARP1.

In some embodiments, provided compounds are useful as PARP1 inhibitors. In some embodiments, the present disclosure provides methods of inhibiting PARP1 in a subject comprising administering a provided compound or composition. In some embodiments, the present disclosure provides methods of inhibiting PARP1 in a biological sample comprising contacting the sample with a provided compound or composition.

In some embodiments, the present disclosure provides methods of treating a disease, disorder or condition associated with PARP1 in a subject in need thereof, comprising administering to the subject a provided compound or composition. In some embodiments, a disease, disorder or condition is associated with overexpression of PARP1. In some embodiments, the present disclosure provides methods of treating a disease, disorder or condition, wherein an underlying pathology is, wholly or partially, mediated by PARP1, in a subject in need thereof, comprising administering to the subject a provided compound or composition.

In some embodiments, the present disclosure provides methods of treating cancer, comprising administering a provided compound or composition to a subject in need thereof. In some embodiments, the present disclosure provides methods of treating proliferative diseases, comprising administering a provided compound or composition to a subject in need thereof. In some embodiments, the present disclosure provides methods of treating metastatic cancers, comprising administering a provided compound or composition to a subject in need thereof. Exemplary cancers include but are not limited to breast cancer, ovarian cancer, cervical cancer, epithelial ovarian cancer, fallopian tube cancer, primary peritoneal cancer, endometrial cancer, prostate cancer, testicular cancer, pancreatic cancer, esophageal cancer, head and neck cancer, gastric cancer, bladder cancer, lung cancer (e.g., adenocarcinoma, non-small-cell lung carcinoma (NSCLC) and small-cell lung carcinoma (SCLC)), bone cancer (e.g., osteosarcoma), colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma (e.g., liposarcoma), bladder cancer, uterine serous carcinoma, liver cancer (e.g., hepatocellular carcinoma), kidney cancer (e.g., renal cell carcinoma), myeloid disorders (e.g., acute myelogenous leukemia (AML), chronic myelogenous leukemia (CML), myelodysplastic syndrome and promyelocytic leukemia), and lymphoid disorders (e.g., leukemia, multiple myeloma, mantle cell lymphoma, acute lymphocytic leukemia (ALL), chronic lymphocytic leukemia (CLL), B-cell lymphoma, T-cell lymphoma, Hodgkin's lymphoma, and non-Hodgkin's lymphoma, hairy cell lymphoma).

When used as a single agent for monotherapy, provided compounds and compositions of the present disclosure are expected to selectively kill tumor cells characterized by homologous recombination deficiency while generating minimal impact on normal tissues. In some embodiments, the present disclosure provides methods of treating advanced cancer induced by or correlated with a dysregulated DNA repair system, comprising administering a provided compound or composition to a subject in need thereof. In some embodiments, such advanced cancers include but are not limited to breast cancer, ovarian cancer, pancreatic cancer, and prostate cancer. These malignant tumors are features of deleterious or suspected deleterious mutations of key genes involved in DNA damage repair pathways. In some embodiments, such key genes include but are not limited to ATM, ATR, BAP1, BRCA1, BRCA2, CDK12, CHEK2, FANCA, FANCC, FANCD2, FANCE, FANCF, PALB2, NBS1, WRN, RAD51C, RAD51D, MRE11A, CHEK1, BLM, RAD51B, and BRIP1. Cancer patients with such mutations can be identified using companion diagnostics. Advanced cancer patients with a positive status of homologous recombination deficiency are expected to benefit from monotherapy with provided compounds and compositions of the present disclosure.

When used as a frontline maintenance therapy, provided compounds and compositions of the present disclosure are useful in treating cancer featured by dysregulated DNA damage repair. Exemplary cancers include but are not limited to triple-negative breast cancer, high-grade serous ovarian cancer, platinum-sensitive advanced pancreatic cancer, and castration-resistant prostate cancer. These tumors are typically sensitive to platinum-based therapies and other DNA damaging agents. As a maintenance therapy, provided compounds and compositions of the present disclosure may reduce risks of recurrence or relapse and therefore prolong progression free survival of patients with advanced cancers.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapy

Cancer cell growth and survival can be impacted by dysfunction in multiple signaling pathways. It is useful to combine compounds modulating different biological targets to treat such conditions. Targeting more than one signaling pathway or more than one biological molecule involved in a given signaling pathway also may reduce the likelihood of drug resistance.

In some embodiments, a provided compound or composition is administered as part of a combination therapy. As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

For example, in some embodiments, a provided compound or composition is administered to a subject who is receiving or has received one or more additional therapies (e.g., an anti-cancer therapy and/or therapy to address one or more side effects of such anti-cancer therapy, or otherwise to provide palliative care).

Exemplary additional therapies include but are not limited to chemotherapies, radiotherapies, anti-inflammatory agents, steroids, immunosuppressants, immune-oncology agents, metabolic enzyme inhibitors, chemokine receptor inhibitors, phosphatase inhibitors, and targeted therapies such as kinase inhibitors.

In some embodiments, a provided compound or composition of the present disclosure can be combined with one or more agents targeting the following biological targets, including but not limiting to Wee1, ATR, ATM, DNA-PK, CDK4/6, CHK1/2, HER2, PI3K, mTOR, EGFR, VEGFR, FGFR, PDGFR, BTK, IGF-1R, BRAF, MEK, KRAS, EZH2, BCL2, HSP90, HDAC, Topoisomerases, HIF-2a, androgen receptor, estrogen receptor, proteosome, RAD51, RAD52, POLQ, WRN, PD-1, and PD-L1. Hypoxia induced by HIF-2a inhibition results in down-regulated expression of the BRCA gene, consequently making tumor cells more vulnerable to PARP1 inhibition. Exemplary cancers for combination of PARP1 and HIF-2a inhibitors include but not limited to clear cell renal cell carcinoma, particularly for the subgroup with the tumor suppressor von Hippel Lindau (VHL) deficiency.

In some embodiments, a provided compound or composition of the present disclosure can be combined with chemotherapies for treatment of cancer. In some embodiments, a provided compound or composition of the present disclosure can be combined with chemotherapies for treatment of high-grade serous ovarian cancer. Exemplary chemotherapies include but are not limited to platinum-based therapy, taxane-based therapy and some others including albumin bound paclitaxel, altretamine, capecitabine, cyclophosphamide, gemcitabine, ifosfamide, irinotecan, liposomal doxorubicin, melphalan, pemetrexed, topotecan, and vinorelbine.

In some embodiments, a provided compound or composition of the present disclosure can be combined with chemotherapies for treatment of advanced metastatic breast cancer. Exemplary chemotherapies include but are not limited to taxanes such as paclitaxel, docetaxel, and albumin-bound paclitaxel, anthracyclines, platinum agents, vinorelbine, capecitabine, gemcitabine, ixabepilone, and eribulin. In some embodiments, such combination therapies can be used for malignancies derived from other histologies, including but limited to brain, lung, kidney, liver, and hematologic cancers.

Radiotherapies are widely used in clinic for treatment of cancers. Provided compounds and compositions of the present disclosure may improve the effectiveness of radiation therapy through its potent activity in suppressing DNA damage repair. In some embodiments, a provided compound or composition of the present disclosure can be combined with radiotherapies for treatment of cancer. Exemplary cancers that can be treated with radiotherapies include but are not limited to small cell lung cancer, leukemias, lymphomas, germ cell tumors, non-melanoma skin cancer, head and neck cancer, breast cancer, non-small cell lung cancer, cervical cancer, anal cancer, and prostate cancer. In some embodiments, provided compounds or compositions of the present disclosure may overcome the resistance of certain cancer to radiotherapy, particularly for renal cell carcinoma and melanomas.

Immunotherapies including antibodies of PD1, PD-L1, and CTLA4 have been successfully used for treatment of cancer. Despite this huge success, resistance and relapse remain a challenge for the vast majority of cancer patients. In some embodiments, a provided compound or composition of the present disclosure can be combined with immunotherapies to improve the effectiveness of conventional antibody-medicated immunotherapies by promoting DNA damage, increasing mutation burden, and modulating the STING innate immune pathway. In some embodiments, a provided compound or composition of the present disclosure can be combined with immunotherapies for treatment of adult and pediatric patients with unresectable or metastatic tumors. In some embodiments, a provided compound or composition of the present disclosure can be combined with immunotherapies for treatment of cancer. Exemplary cancers include but are not limited to non-small cell lung cancer, melanoma, head and neck squamous cell carcinoma, classical Hodgkin lymphoma, urothelial carcinoma, microsatellite instability-high cancer, gastric cancer, cervical cancer, primary mediastinal large B-cell lymphoma, hepatocellular carcinoma, Merkel cell carcinoma, renal cell carcinoma, esophageal cancer, endometrial cancer, tumor mutational burden-high cancer, cutaneous squamous cell carcinoma, microsatellite instability-high or mismatch repair deficient colorectal cancer, and triple-negative breast cancer.

In some embodiments, a provided compound or composition of the present disclosure can be combined with targeted therapies of well-established therapeutic targets including but not limited to PI3K inhibitors, KRAS inhibitors, CDK4/6 inhibitors, BRAF inhibitors, MEK inhibitors, androgen receptor inhibitors, selective estrogen receptor modulators, proteosome inhibitors, mTOR inhibitors, EGFR inhibitors, FGFR inhibitors, MET inhibitors, PDGFR inhibitors, VEGFR inhibitors, EZH2 inhibitors, BTK inhibitors, and BCL2 inhibitors for treatment of cancer. Exemplary cancers include but are not limited to breast cancer, ovarian cancer, non-small cell lung cancer, hepatocellular carcinoma, clear cell renal cell carcinoma, melanoma, colorectal cancer, bladder cancer, prostate cancer, cholangiocarcinoma, and hematologic cancers.

In some embodiments, a provided compound or composition of the present disclosure can be combined with inhibitors of other DNA damage repair proteins including but not limited to CHEK1, CHEK2, ATM, ATR, DNA-PK, WEE1, RAD51, RAD52, POLQ, and WRN for treatment of cancer sensitive to DNA damage. In some embodiments, a provided compound or composition of the present disclosure can be combined with a WEE1 inhibitor for treatment of uterine serous carcinoma and cancers with mutation of the TP53 genes. In some embodiments, a provided compound or composition of the present disclosure can be combined with a WRN inhibitor for treatment of microsatellite instability-high cancers, such as colon cancer, gastric cancer, endometrium cancer, ovarian cancer, hepatobiliary tract cancer, urinary tract cancer, brain cancer, and skin cancers.

### Labeled Compounds and Assay Methods

Another aspect of the present invention relates to fluorescent dye, spin label, heavy metal or radio-labeled compounds of the invention that would be useful not only in imaging but also in assays, both in vitro and in vivo, for localizing and quantitating the PARP1 enzyme in tissue samples, including human, and for identifying PARP1 enzyme ligands by inhibition binding of a labeled compound. Accordingly, the present invention includes PARP1 enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro FGFR enzyme labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

One or more constituent atoms of the compounds presented herein can be replaced or substituted with isotopes of the atoms in natural or non-natural abundance. In some embodiments, one or more atoms are replaced or substituted by deuterium. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ being substituted for —CH₃). In some embodiments, alkyl groups of the disclosed Formulas (e.g., the compound of any of Formulas I, II, II-a, II-a-i, III, IV, V, VI, VI-a, VI-b, VII, VIII, VIII-a, VIII-b, VIII-c, VIII-d, IX, IX-a, IX-b, IX-c, and IX-d) can be perdeuterated.

In some embodiments, the compound provided herein (e.g., the compound of any of Formulas I, II, II-a, II-a-i, III, IV, V, VI, VI-a, VI-b, VII, VIII, VIII-a, VIII-b, VIII-c, VIII-d, IX, IX-a, IX-b, IX-c, and IX-d), or a pharmaceutically acceptable salt thereof, comprises at least one deuterium atom.

In some embodiments, the compound provided herein (e.g., the compound of any of Formulas I, II, II-a, II-a-i, III, IV, V, VI, VI-a, VI-b, VII, VIII, VIII-a, VIII-b, VIII-c, VIII-d, IX, IX-a, IX-b, IX-c, and IX-d), or a pharmaceutically acceptable salt thereof, comprises two or more deuterium atoms.

In some embodiments, the compound provided herein (e.g., the compound of any of Formulas I, II, II-a, II-a-i, III, IV, V, VI, VI-a, VI-b, VII, VIII, VIII-a, VIII-b, VIII-c, VIII-d, IX, IX-a, IX-b, IX-c, and IX-d), or a pharmaceutically acceptable salt thereof, comprises three or more deuterium atoms.

In some embodiments, for a compound provided herein (e.g., the compound of any of Formulas I, II, II-a, II-a-i, III, IV, V, VI, VI-a, VI-b, VII, VIII, VIII-a, VIII-b, VIII-c, VIII-d, IX, IX-a, IX-b, IX-c, and IX-d), or a pharmaceutically acceptable salt thereof, all of the hydrogen atoms are replaced by deuterium atoms (i.e., the compound is “perdeuterated”).

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the PARP1 enzyme. Accordingly, the ability of a test compound to compete with the radio-labeled compound for binding to the PARP1 enzyme directly correlates to its binding affinity.

### Kits

The present invention also includes pharmaceutical kits useful, for example, in the treatment or prevention of PARP1-associated diseases or disorders referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples were found to be inhibitors of PARP1 as described below.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

### Materials and Methods

The final compounds were purified on a preparative scale reverse-phase high performance liquid chromatography (RP-HPLC) or flash chromatography (silica gel) as indicated in the Examples. Typical preparative reverse-phase high performance liquid chromatography (RP-HPLC) column conditions are as follows:

TFA conditions: column, Waters XSelect CSH C₁₈ 5 μm particle size, 30×150 mm; eluting with mobile phase A: water (0.05% trifluoroacetic acid), mobile Phase B: acetonitrile; the flow rate, 60 mL/min.

NH₄HCO₃ conditions: column, waters XBridge BEH C₁₈ 5 μm particle size, 30×150 mm; eluting with mobile phase A: water (10 mM ammonium bicarbonate), mobile Phase B: acetonitrile; the flow rate, 60 mL/min.

HCOOH conditions: column, Sunfire Prep C₁₈ OBD 5 μm particle size, 30×150 mm; eluting with mobile phase A: water (0.1% formic acid), mobile Phase B: acetonitrile; the flow rate, 60 mL/min.

The separating gradient was optimized for each compound. The separated compounds were typically subjected to analytical liquid chromatography mass spectrometry (LCMS) for purity check under the following conditions: Instrument: Shimadzu LCMS-2020, column: Halo C18 2 μm particle size, 3×30 mm; buffers: mobile phase A: 0.05% TFA in water and mobile phase B: acetonitrile; gradient: 0 to 60% of B in 1.9 min, 60% to 100% of B in 0.35 min with flow rate 1.5 mL/min.

### Preparation of Intermediates

### Intermediate 1: N-methyl-5-(piperazin-1-yl)picolinamide dihydrochloride

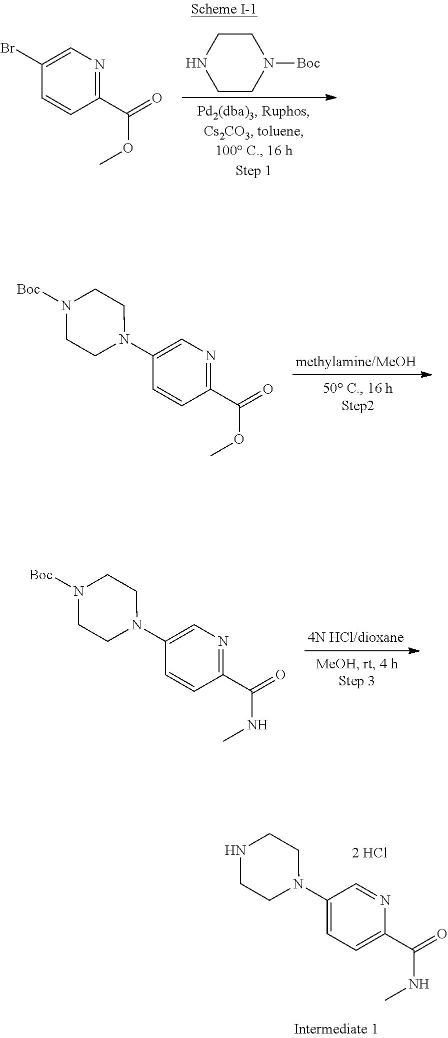 [SMILES: COC(=O)c1ccc(Br)cn1 ; CC(C)(C)OC(=O)N1CCNCC1 ; COC(=O)c1ccc(N2CCN(C(=O)OC(C)(C)C)CC2)cn1 ; CNC(=O)c1ccc(N2CCN(C(=O)OC(C)(C)C)CC2)cn1 ; CNC(=O)c1ccc(N2CCNCC2)cn1]

### Step 1: tert-butyl 4-(6-(methoxycarbonyl)pyridin-3-yl)piperazine-1-carboxylate

The mixture of methyl 5-bromopyridine-2-carboxylate (5 g, 23.1 mmol), tert-butyl piperazine-1-carboxylate (4.53 g, 24.3 mmol), cesium carbonate (22.62 g, 69.4 mmol), tris(dibenzylideneacetone) dipalladium (1.06 g, 1.2 mmol) and 2-dicyclohexylphosphino-2′,6′-diisopropoxy-1,1′-biphenyl (1.08 g, 2.3 mmol) in toluene (200 mL) was heated at 100° C. for 16 h under a nitrogen atmosphere. Upon cooling to room temperature, the mixture was concentrated under reduced pressure, and the residue was purified by silica gel column chromatography, eluted with 50% ethyl acetate in petroleum ether to provide the desired product as a yellow solid (4.7 g, 63%). LCMS calculated for C₁₆H₂₄N₃O₄ (M+H)⁺ m/z=322.2; found 322.1; ¹H NMR (300 MHz, CDCl₃) δ 8.33 (d, J=2.7 Hz, 1H), 8.00 (d, J=9.0 Hz, 1H), 7.15 (dd, J=8.7, 3.0 Hz, 1H), 3.95 (s, 3H), 3.62-3.58 (m, 4H), 3.35-3.32 (m, 4H), 1.47 (s, 9H).

### Step 2: tert-butyl 4-(6-(methylcarbamoyl)pyridin-3-yl)piperazine-1-carboxylate

The mixture of tert-butyl 4-(6-(methoxycarbonyl)pyridin-3-yl)piperazine-1-carboxylate (4.7 g, 14.6 mmol) in methylamine (2 M in methanol, 100 mL) was stirred at 50° C. in sealed tube for 16 h. Upon cooling to room temperature, the mixture was concentrated under reduced pressure to give the desired product as a white solid (4.3 g, 92%). LCMS calculated for C₁₆H₂₅N₄O₃ (M+H)⁺ m/z=321.2; found 321.2; ¹H NMR (400 MHz, CDCl₃) δ 8.16 (d, J=2.8 Hz, 1H), 8.06 (d, J=8.4 Hz, 1H), 8.00 (brs, 1H), 7.22 (dd, J=8.8, 2.8 Hz, 1H), 3.62-3.60 (m, 4H), 3.30-3.28 (m, 4H), 3.01 (d, J=5.2 Hz, 3H), 1.49 (s, 9H).

### Step 3: N-methyl-5-(piperazin-1-yl)picolinamide dihydrochloride

The mixture of tert-butyl 4-(6-(methylcarbamoyl)pyridin-3-yl)piperazine-1-carboxylate (4.3 g, 13.4 mmol) and hydrogen chloride (4 M in 1,4-dioxane, 30 mL) in methanol (10 mL) was stirred at 0° C. for 2 h under nitrogen atmosphere. The mixture was concentrated under vacuum and the residue was diluted with the mixture of diethyl ether and hexane (1/1, 30 mL). The solid was collected by filtration, washed with hexanes, dried under vacuum to give the desired product as a white solid (3.9 g, 99%). LCMS calculated for C₁₁H₁₇N₄O (M+H)⁺ m/z=221.1; found 221.3; ¹H NMR (300 MHz, DMSO-d₆) δ 9.88 (s, 2H), 9.15 (s, 1H), 8.33 (d, J=3.0 Hz, 1H), 8.28 (d, J=8.7 Hz, 1H), 7.84 (dd, J=9.0, 3.0 Hz, 1H), 3.74-3.71 (m, 4H), 3.21-3.18 (m, 4H), 2.81 (s, 3H).

### Intermediate 2: N,6-dimethyl-5-(piperazin-1-yl)picolinamide hydrochloride

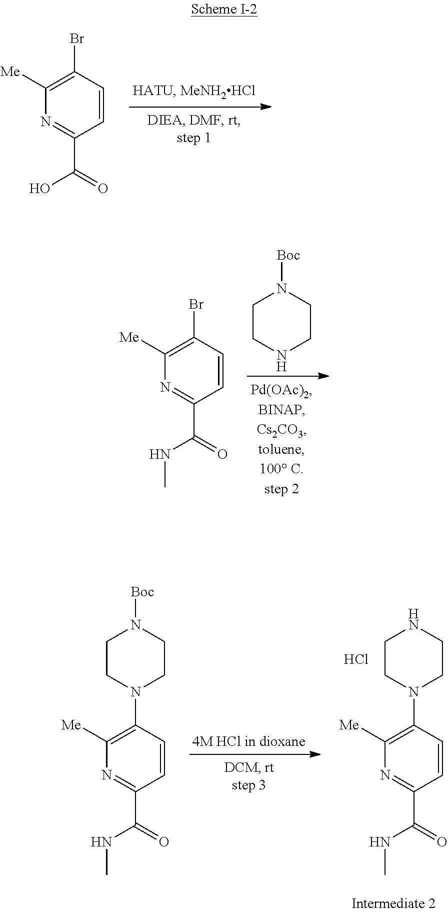

### Step 1: 5-Bromo-N,6-dimethylpicolinamide

The mixture of 5-bromo-6-methylpyridine-2-carboxylic acid (5 g, 23.1 mmol) was treated with O-(7-Azabenzotriazol-1-yl)-N,N,N,N-tetramethyl uronium hexafluorophosphate (10.56 g, 27.8 mmol) in N,N-dimethylformamide (80 mL) at room temperature for 30 min, followed by the addition of N-ethyl-N-isopropylpropan-2-amine (14.96 g, 115.7 mmol) and methylamine hydrochloride (2.34 g, 34.7 mmol). The resulting mixture was stirred at the same temperature for 2 h, and then diluted with ethyl acetate (500 mL). The resulting mixture was washed with water (3×100 mL) and brine (3×100 mL). The combined organics were dried with anhydrous sodium sulfate. After filtered, the filtrate was concentrated under reduced pressure. The residue was purified by silica gel column chromatography, eluted with 50% ethyl acetate in petroleum ether to provide the desired product as a white solid (4.53 g, 85%). LCMS calculated for C₈H₁₀BrN₂O (M+H)⁺ m/z=229.0; found 228.9, 230.9; ¹H NMR (300 MHz, CD₃OD) δ 8.11 (d, J=8.1 Hz, 1H), 7.81 (d, J=8.1 Hz, 1H), 2.97 (s, 3H), 2.72 (s, 3H).

### Step 2: tert-Butyl 4-(2-methyl-6-(methylcarbamoyl)pyridin-3-yl)piperazine-1-carboxylate

The mixture of 5-bromo-N,6-dimethylpyridine-2-carboxamide (700 mg, 3.1 mmol), tert-butyl piperazine-1-carboxylate (854 mg, 4.6 mmol), palladium(II) acetate (69 mg, 0.31 mmol), racemic-2,2′-Bis(diphenylphosphino)-1,1′-binaphthyl (285 mg, 0.46 mmol), and cesium carbonate (1.99 g, 6.1 mmol) in toluene (12 mL) was stirred at 80° C. for 18 h under nitrogen atmosphere. After cooling to room temperature, the resulting mixture was concentrated under reduced pressure, and the residue was purified by silica gel column chromatography, eluted with 60% ethyl acetate in petroleum ether to afford the desired product as a yellow solid (800 mg, 78%). LCMS calculated for C₁₇H₂₇N₄O₃ (M+H)⁺ m/z=335.2; found 335.3; ¹H NMR (300 MHz, CDCl₃) δ 8.12 (brs, 1H), 8.04 (d, J=8.4 Hz, 1H), 7.38 (d, J=8.4 Hz, 1H), 3.64-3.61 (m, 4H), 3.04 (d, J=5.1 Hz, 3H), 2.95-2.92 (m, 4H), 2.58 (s, 3H), 1.51 (s, 9H).

### Step 3: N,6-dimethyl-5-(piperazin-1-yl)picolinamide hydrochloride

The mixture of tert-butyl 4-[2-methyl-6-(methylcarbamoyl)pyridin-3-yl]piperazine-1-carboxylate (150 mg, 0.45 mmol) in dichloromethane (6 mL) was treated with hydrogen chloride (4 mL, 4 M in dioxane). After stirring at room temperature for 2 h, the mixture was concentrated under reduced pressure to provide the desired product as a light-yellow solid (130 mg, crude) which was used in the next step without further purification. LCMS calculated for C₁₂H₁₉N₄O (M+H)⁺ m/z=235.2; found 235.1.

### Intermediate 3: N-methyl-5-(piperazin-1-yl)-6-(trifluoromethyl)picolinamide hydrochloride

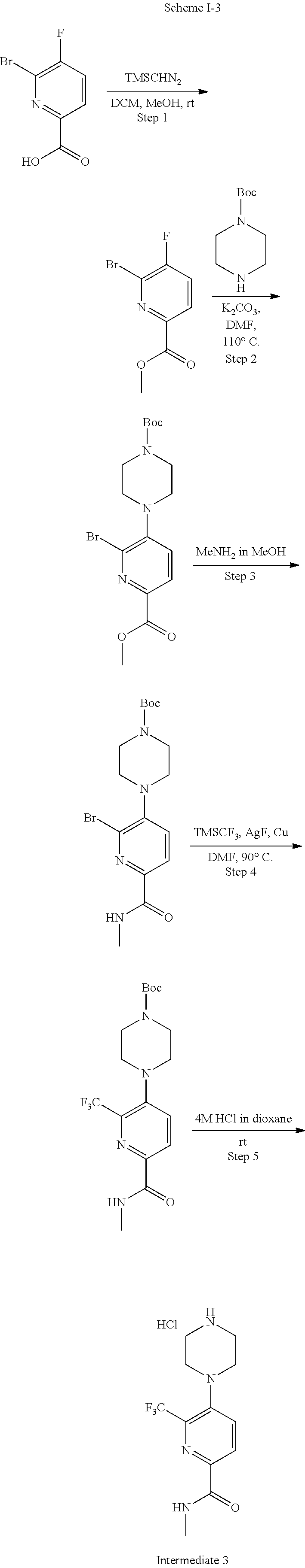

### Step 1: Methyl 6-bromo-5-fluoropicolinate

The mixture of 6-bromo-5-fluoropicolinic acid (5 g, 22.73 mmol) in dichloromethane (50 mL) and methanol (50 mL) was combined with (trimethylsilyl)diazomethane (2M in hexane, 45.46 mL, 90.91 mmol) at room temperature for 16 h under nitrogen atmosphere, and then quenched with saturated aqueous sodium carbonate (100 mL), extracted with ethyl acetate (2×100 mL) and dried with anhydrous sodium sulfate. After filtration, the filtrate was concentrated under reduced pressure. The residue was purified by silica gel column chromatography, eluted with 30% ethyl acetate in petroleum to provide the desired product as a white solid (4.6 g, 86%). LCMS calculated for C₇H₆BrFNO₂ (M+H)⁺ m/z=234.0; found 234.0.

### Step 2: tert-Butyl 4-(2-bromo-6-(methoxycarbonyl)pyridin-3-yl)piperazine-1-carboxylate

The mixture of methyl 6-bromo-5-fluoropicolinate (1.71 g, 7.31 mmol), potassium carbonate (2.02 g, 14.61 mmol) and tert-butyl piperazine-1-carboxylate (1.43 g, 7.67 mmol) in N,N-dimethylformamide (20 mL) was stirred at 110° C. for 5 h. Upon cooling to room temperature, the mixture was diluted with water (200 mL), extracted with ethyl acetate (3×100 mL). The combined organic layers were washed with brine (3×100 mL), dried with anhydrous sodium sulfate. After filtration, the filtrate was concentrated under reduced pressure. The residue was purified by silica gel column chromatography, eluted with 20% ethyl acetate in dichloromethane to provide the desired product as a yellow solid (1.83 g, 63%). LCMS calculated for C₁₆H₂₃BrN₃O₄ (M+H)⁺ m/z=400.1; found 400.1; ¹H NMR (400 MHz, CDCl₃) δ 8.06 (d, J=8.0 Hz, 1H), 7.30 (d, J=8.4 Hz, 1H), 3.97 (s, 3H), 3.66-3.63 (m, 4H), 3.13-3.10 (m, 4H), 1.49 (s, 9H).

### Step 3: tert-Butyl 4-(2-bromo-6-(methylcarbamoyl)pyridin-3-yl)piperazine-1-carboxylate

The mixture of tert-butyl 4-(2-bromo-6-(methoxycarbonyl)pyridin-3-yl)piperazine-1-carboxylate (1.83 g, 4.57 mmol) was combined with methylamine (30 mL, 31% in methanol) at room temperature, and stirred at the same temperature for 16 h. The mixture was then concentrated under reduced pressure. The residue was purified by silica gel column chromatography, eluted with 25% ethyl acetate in dichloromethane to provide the desired product as a yellow solid as a yellow solid (1.6 g, 88%). LCMS calculated for C₁₆H₂₄BrN₄O₃ (M+H)⁺ m/z=399.1; found 399.1; ¹H NMR (300 MHz, CDCl₃) δ 8.12 (dd, J=8.1 Hz, 1H), 7.70 (s, 1H), 7.37 (d, J=8.1 Hz, 1H), 3.68-3.64 (m, 4H), 3.11-3.08 (m, 4H), 3.04 (d, J=5.0 Hz, 3H), 1.51 (s, 9H).

### Step 4: tert-Butyl 4-(6-(methylcarbamoyl)-2-(trifluoromethyl)pyridin-3-yl)piperazine-1-carboxylate

The mixture of tert-butyl 4-(2-bromo-6-(methylcarbamoyl)pyridin-3-yl)piperazine-1-carboxylate (1.6 g, 4.01 mmol), silver fluoride (1.83 g, 14.43 mmol) and copper powder (1.40 g, 22.04 mmol) in N,N-dimethylformamide (20 mL) was stirred at room temperature for 2 h, followed by the addition of trifluoromethyltrimethylsilane (2.51 g, 17.63 mmol) in portions at room temperature. The resulting mixture was stirred at 90° C. for 18 h. Upon cooling to room temperature, the mixture was diluted with water (150 mL), extracted with ethyl acetate (3×100 mL). The combined organic layers were washed with brine (3×100 mL) and dried with anhydrous sodium sulfate. After filtration, the filtrate was concentrated under reduced pressure. The residue was purified by silica gel column chromatography, eluted with 30% dichloromethane in ethyl acetate to provide the desired product as a light-yellow solid (880 mg, 57%). LCMS calculated for C₁₇H₂₄F₃N₄O₃ (M+H)⁺ m/z=389.2; found 389.3; ¹H NMR (400 MHz, CDCl₃) δ 8.32 (d, J=8.4 Hz, 1H), 7.81 (s, 1H), 7.70 (d, J=8.4 Hz, 1H), 3.61-3.59 (m, 4H), 3.04 (d, J=5.0 Hz, 3H), 3.00-2.97 (m, 4H), 1.49 (s, 9H).

### Step 5: N-methyl-5-(piperazin-1-yl)-6-(trifluoromethyl)picolinamide hydrochloride

The mixture of tert-butyl 4-(6-(methylcarbamoyl)-2-(trifluoromethyl)pyridin-3-yl)piperazine-1-carboxylate (30 mg, 0.08 mmol) in hydrochloride (4 M in 1,4-dioxane, 0.5 mL) was stirred at room temperature for 1 h, and then concentrated under reduced pressure to provide the desired product as a yellow solid (20 mg, crude) which was used in the next step without further purification. LCMS calculated for C₁₂H₁₆F₃N₄O (M+H)⁺ m/z=289.1; found 289.3.

### Intermediate 4: 6-Fluoro-N-methyl-5-(piperazin-1-yl)picolinamide hydrochloride

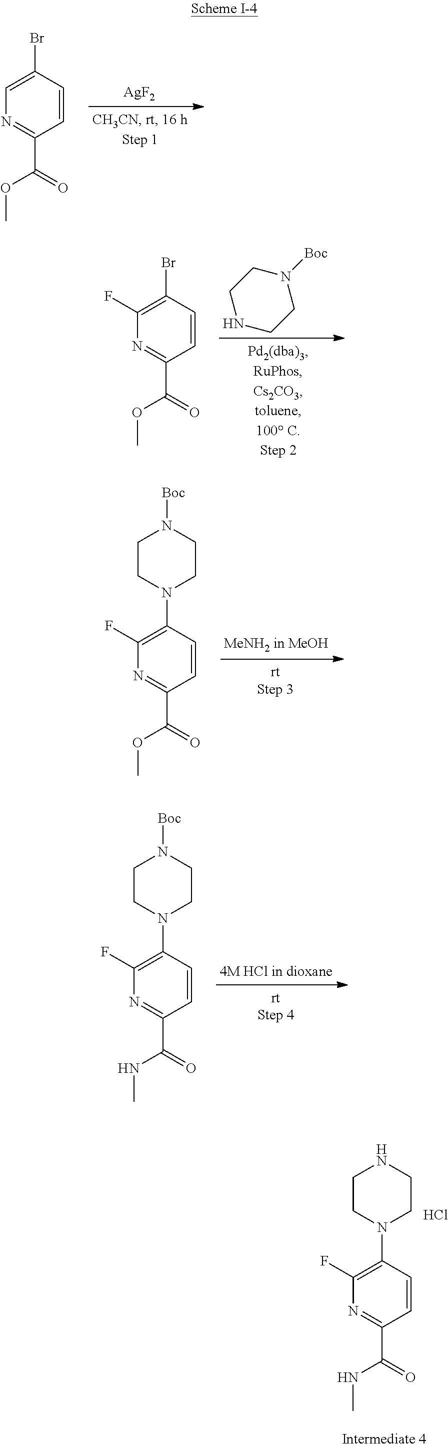

### Step 1: Methyl 5-bromo-6-fluoropicolinate

The mixture of methyl 5-bromopicolinate (5 g, 23.15 mmol) and difluorosilver (11.82 g, 81.01 mmol) in acetonitrile (50 mL) was stirred at room temperature for 16 h. The mixture was filtered, and the filter-cake was washed with dichloromethane (2×100 mL). The filtrate was washed with saturated aqueous ammonium chloride (200 mL) and dried with anhydrous sodium sulfate. After filtration, the filtrate was concentrated under reduced pressure. The residue was purified by silica gel column chromatography, eluted with 30% ethyl acetate in petroleum ether to provide the desired product as a white solid (4.52 g, 83%). LCMS calculated for CH₆BrFNO₂ (M+H)⁺ m/z=234.0; found 233.9; ¹H NMR (300 MHz, CDCl₃) δ 8.14 (dd, J=10.8, 10.8 Hz, 1H), 7.92 (d, J=10.8 Hz, 1H), 4.00 (s, 3H); ¹⁹F NMR (377 MHz, CDCl₃) δ −61.98.

### Step 2: tert-Butyl 4-(2-fluoro-6-(methoxycarbonyl)pyridin-3-yl)piperazine-1-carboxylate

The mixture of methyl 5-bromo-6-fluoropicolinate (3.3 g, 14.1 mmol), tert-butyl piperazine-1-carboxylate (3.94 g, 21.15 mmol), 2-dicyclohexylphosphino-2′,6′-diisopropoxy-1,1′-biphenyl (0.99 g, 2.12 mmol), tris(dibenzylideneacetone) dipalladium (1.29 g, 1.41 mmol) and cesium carbonate (9.19 g, 28.2 mmol) in toluene (40 mL) was stirred at 100° C. for 16 h under nitrogen atmosphere. Upon cooling to room temperature, the mixture was concentrated under reduced pressure. The residue was purified by silica gel column chromatography, eluted with 50% ethyl acetate in petroleum ether to provide the desired product as a yellow solid (2.7 g, 56%). LCMS calculated for C₁₆H₂₃FN₃O₄ (M+H)⁺ m/z=340.2; found 340.1; ¹H NMR (400 MHz, CDCl₃) δ 7.97 (dd, J=8.0, 1.6 Hz, 1H), 7.28-7.23 (m, 1H), 3.96 (s, 3H), 3.63-3.60 (m, 4H), 3.22-3.19 (m, 4H), 1.49 (s, 9H); 1° F. NMR (282 MHz, CDCl₃) δ −69.20.

### Step 3: tert-Butyl 4-(2-fluoro-6-(methylcarbamoyl)pyridin-3-yl)piperazine-1-carboxylate

The mixture of tert-butyl 4-(2-fluoro-6-(methoxycarbonyl)pyridin-3-yl)piperazine-1-carboxylate (2.7 g, 7.96 mmol) was combined with methylamine (30 mL, 31% in methanol) at room temperature, and stirred at the same temperature for 16 h. The mixture was then concentrated under reduced pressure. The residue was purified by silica gel column chromatography, eluted with 30% ethyl acetate in dichloromethane to provide the desired product as a yellow solid (2.3 g, 85%). LCMS calculated for C₁₆H₂₄FN₄O₃ (M+H)⁺ m/z=339.2; found 339.3; ¹H NMR (300 MHz, CDCl₃) δ 8.02 (dd, J=8.1, 1.5 Hz, 1H), 7.52 (s, 1H), 7.37-7.30 (m, 1H), 3.65-3.61 (m, 4H), 3.18-3.15 (m, 4H), 3.02 (d, J=5.0 Hz, 3H), 1.51 (s, 9H); ¹⁹F NMR (282 MHz, CDCl₃) δ −72.71.

### Step 4: 6-Fluoro-N-methyl-5-(piperazin-1-yl)picolinamide hydrochloride

The mixture of tert-butyl 4-(2-fluoro-6-(methylcarbamoyl)pyridin-3-yl)piperazine-1-carboxylate (28 mg, 0.08 mmol) in hydrochloride (4 M in 1,4-dioxane, 0.9 mL) was stirred at room temperature for 2 h, and then concentrated under reduced pressure to provide the desired product as a yellow solid (20 mg, crude) which was used in the next step without further purification. LCMS calculated for C₁₁H₁₆FN₄O (M+H)⁺ m/z=239.1; found 239.2.

### Intermediate 5: N-ethyl-6-methyl-5-(piperazin-1-yl)picolinamide hydrochloride

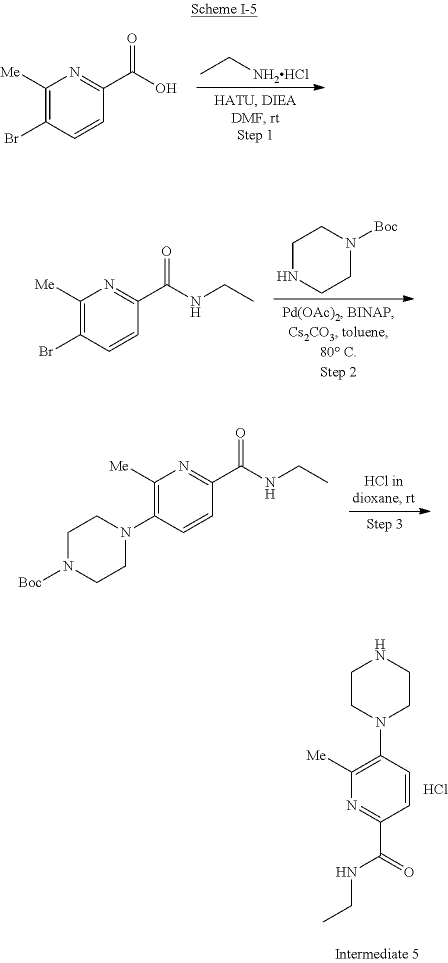

### Step 1: 5-Bromo-N-ethyl-6-methylpicolinamide

The mixture of 5-bromo-6-methylpicolinic acid (2 g, 9.26 mmol) in N,N-dimethylformamide (30 mL) was treated with 2-(7-azabenzotriazol-1-yl)-N,N,N′,N′-tetramethyluronium hexafluorophosphate (4.22 g, 11.11 mmol) at room temperature for 30 min, followed by the addition of ethanamine hydrochloride (1.13 g, 13.89 mmol) and N-ethyl-N-isopropylpropan-2-amine (5.98 g, 46.29 mmol). The resulting mixture was stirred at the same temperature for 16 h, and then diluted with ethyl acetate (300 mL). The resulting mixture was washed with water (3×100 mL) and brine (3×100 mL). The combined organics were dried with anhydrous sodium sulfate. After filtration, the filtrate was concentrated under reduced pressure. The residue was purified by silica gel column chromatography, eluted with 50% ethyl acetate in petroleum ether to provide the desired product as a white solid (1.5 g, 67%). LCMS calculated for C₉H₁₂BrN₂O (M+H)⁺ m/z=243.0; found 242.9; ¹H NMR (300 MHz, CDCl₃) δ 7.97-7.88 (m, 3H), 3.55-3.46 (m, 2H), 2.69 (s, 3H), 1.28 (t, J=7.2 Hz, 3H).

### Step 2: Tert-butyl 4-(6-(ethylcarbamoyl)-2-methylpyridin-3-yl)piperazine-1-carboxylate

The mixture of 5-bromo-N-ethyl-6-methylpicolinamide (320 mg, 1.32 mmol), tert-butyl piperazine-1-carboxylate (294 mg, 1.58 mmol), palladium acetate (30 mg, 0.13 mmol), racemic-2,2′-bis(diphenylphosphino)-1,1′-binaphthyl (123 mg, 0.2 mmol) and cesium carbonate (858 mg, 2.63 mmol) in dry toluene (8 mL) was stirred at 80° C. for 16 h under a nitrogen atmosphere. After cooling down to room temperature, the mixture was filtered, the filter cake was washed with dichloromethane (3×5 mL). The filtrate was concentrated under reduced pressure. The residue was purified by silica gel column chromatography, eluted with 40% ethyl acetate in petroleum ether to provide the desired product as a white solid (360 mg, 78%). LCMS calculated for C₁₈H₂₉N₄O₃ (M+H)⁺ m/z=349.2; found 349.3; ¹H NMR (300 MHz, CDCl₃) δ 8.07 (brs, 1H), 8.02 (d, J=8.4 Hz, 1H), 7.36 (d, J=8.4 Hz, 1H), 3.62-3.59 (m, 4H), 3.55-3.46 (m, 2H), 2.93-2.89 (m, 4H), 2.57 (s, 3H), 1.49 (s, 9H), 1.27 (t, J=7.2 Hz, 3H).

### Step 3: N-ethyl-6-methyl-5-(piperazin-1-yl)picolinamide hydrochloride

The mixture of tert-butyl 4-(6-(ethylcarbamoyl)-2-methylpyridin-3-yl)piperazine-1-carboxylate (50 mg, 0.14 mmol) in dichloromethane (1 mL) was treated with hydrogen chloride (4 M in 1,4-dioxane, 1.0 mL). After stirring at room temperature for 1 h, the mixture was concentrated under reduced pressure to provide the desired product as a light-yellow solid which was used in the next step without further purification. LCMS calculated for C₁₃H₂₁N₄O (M+H)⁺ m/z=249.2; found 249.1.

### Intermediate 6: N,6-bis(methyl-d₃)-5-(piperazin-1-yl)picolinamide

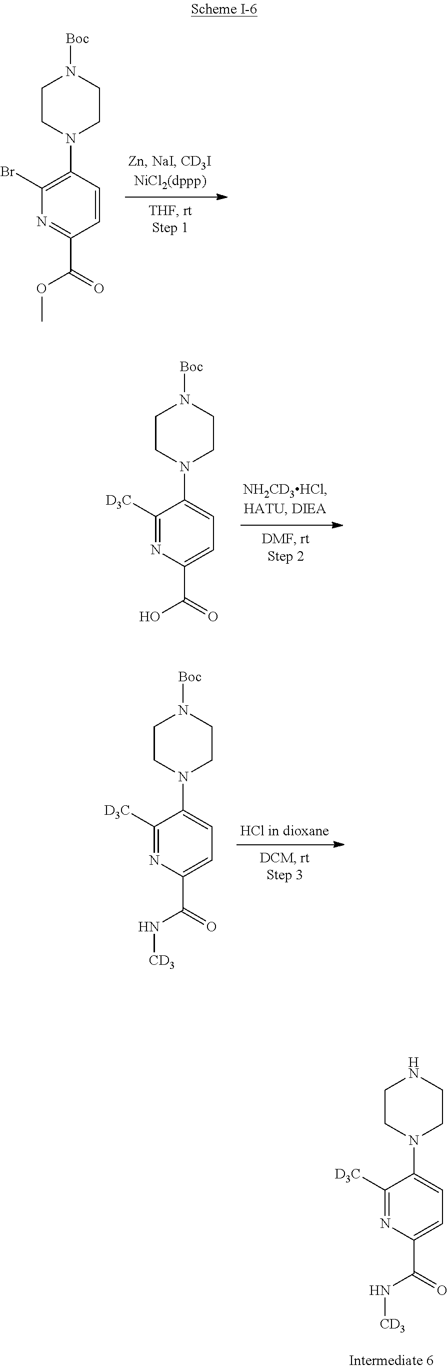 [SMILES: COC(=O)c1ccc(N2CCN(C(=O)OC(C)(C)C)CC2)c(Br)n1 ; [2H]C([2H])([2H])c1nc(C(=O)O)ccc1N1CCN(C(=O)OC(C)(C)C)CC1 ; [2H]C([2H])([2H])NC(=O)c1ccc(N2CCN(C(=O)OC(C)(C)C)CC2)c(C([2H])([2H])[2H])n1 ; [2H]C([2H])([2H])NC(=O)c1ccc(N2CCNCC2)c(C([2H])([2H])[2H])n1]

### Step 1: 5-(4-(Tert-butoxycarbonyl)piperazin-1-yl)-6-(methyl-d₃) picolinic acid

The mixture of zinc (1.63 g, 24.98 mmol), [1,3-bis(diphenylphosphino) propane]nickel (II) chloride (339 mg, 0.63 mmol), sodium iodide (1.4 g, 9.37 mmol), tert-butyl 4-(2-bromo-6-(methoxycarbonyl)pyridin-3-yl)piperazine-1-carboxylate (Intermediate 3, Step 2:2.5 g, 6.25 mmol) and iodomethane-d₃ (4.53 g, 31.23 mmol) in dry tetrahydrofuran (30 mL) was stirred at room temperature for 24 h under nitrogen atmosphere. Then it was concentrated under vacuum. The residue was purified by reverse-phase flash chromatography (column: C18 silica gel; mobile phase: acetonitrile in water: elution: 5% to 70% gradient over 30 min; detector: UV 254 nm); The fractions were collected, combined and lyophilized to provide the desired product (1.0 g, 39%). LCMS calculated for C₁₆H₂₁D₃N₃O₄ (M+H)⁺ m/z=325.2; found 325.1.

### Step 2: Tert-butyl 4-(2-(methyl-d₃)-6-((methyl-d₃)carbamoyl)pyridin-3-yl)piperazine-1-carboxylate

The mixture of 5-(4-(tert-butoxycarbonyl)piperazin-1-yl)-6-(methyl-d₃) picolinic acid (450 mg, 1.39 mmol) in N,N-dimethylformamide (5 mL) was treated with 2-(7-azabenzotriazol-1-yl)-N,N,N′,N′-tetramethyluronium hexafluorophosphate (633 mg, 1.66 mmol) at room temperature for 30 min, followed by the addition of methan-d₃-amine hydrochloride (117 mg, 1.66 mmol) and N-ethyl-N-isopropylpropan-2-amine (538 mg, 4.16 mmol). The resulting mixture was stirred at the same temperature for 2 h, and then diluted with ethyl acetate (50 mL). The resulting mixture was washed with water (3×30 mL) and brine (3×30 mL). The combined organics were dried with anhydrous sodium sulfate. After filtration, the filtrate was concentrated under reduced pressure. The residue was purified by silica gel column chromatography, eluted with 50% ethyl acetate in dichloromethane to provide the desired product as a yellow solid (227 mg, 48%). LCMS calculated for C₁₇H₂₁D₆N₄O₃ (M+H)⁺ m/z=341.2; found 341.2.

### Step 3: N,6-bis(methyl-d₃)-5-(piperazin-1-yl)picolinamide

A solution of tert-butyl 4-(2-(methyl-d₃)-6-((methyl-d₃)carbamoyl)pyridin-3-yl)piperazine-1-carboxylate (227 mg, 0.67 mmol) in dichloromethane (3 mL) was treated with hydrogen chloride (4 M in 1,4-dioxane, 3 mL). After stirring at room temperature for 2 h, the mixture was concentrated under reduced pressure. To the residue was charged a saturated sodium bicarbonate aqueous solution (30 mL). The mixture was extracted with dichloromethane (4×50 mL). The combined organic layers were dried with anhydrous sodium sulfate. After filtered, the filtrate was concentrated under reduced pressure to provide the desired product as a brown solid (150 mg) which was used directly without further purification. LCMS calculated for C₁₂H₁₃D₆N₄O (M+H)⁺ m/z=241.2; found 241.1.

### Intermediate 7: Methyl 6-methyl-5-(piperazin-1-yl) picolinate

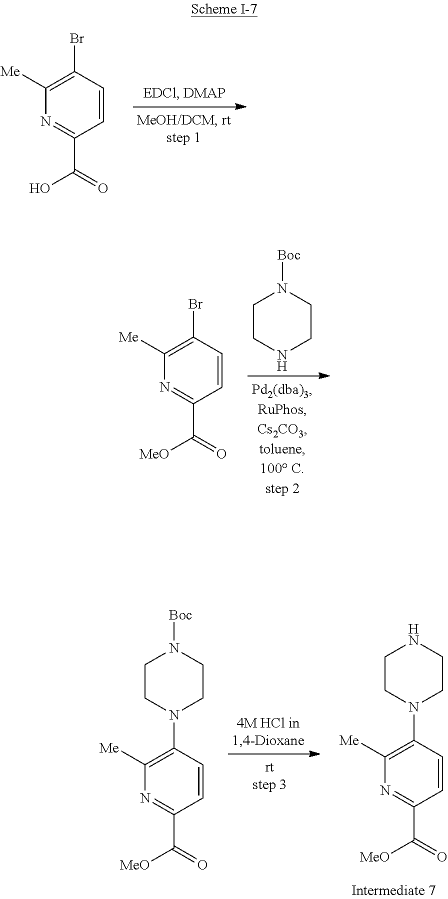

### Step 1: Methyl 5-bromo-6-methylpicolinate

To a stirred mixture of 5-bromo-6-methylpicolinic acid (5 g, 23.15 mmol) in dichloromethane (40 mL) was added N,N-dimethylpyridin-4-amine (4.24 g, 34.71 mmol) at 0° C. 1-Ethyl-3-(3-dimethylaminopropyl) carbodiimide HCl (4.67 g, 24.36 mmol) was added in portions. Then methanol (10 mL, 246.96 mmol) was added. The resulting mixture was stirred at room temperature for additional 16 h. The reaction mixture was diluted with water (100 mL) and extracted with ethyl acetate (3×100 mL). The combined organic layers were dried over anhydrous sodium sulfate. After filtration, the filtrate was concentrated under reduced pressure. The residue was purified by silica gel column chromatography, eluted with 30% ethyl acetate in petroleum ether to give the desired product as a white solid (3.7 g, 69%). LCMS calculated for C₈H₉BrNO₂ (M+H)⁺ m/z=230.0; found 229.9; ¹H NMR (300 MHz, CDCl₃) δ 7.97 (d, J=8.2 Hz, 1H), 7.83 (d, J=8.2 Hz, 1H), 4.00 (s, 3H), 2.77 (s, 3H).

### Step 2: tert-Butyl 4-(6-(methoxycarbonyl)-2-methylpyridin-3-yl)piperazine-1-carboxylate

To a round bottom flask equipped with a magnetic stir bar was added methyl 5-bromo-6-methylpicolinate (3.6 g, 15.65 mmol), cesium carbonate (15.295 g, 46.94 mmol), 2-dicyclohexylphosphino-2′,6′-diisopropoxybiphenyl (1.46 g, 3.13 mmol), tris(dibenzylideneacetone) dipalladium (1.43 g, 1.57 mmol) and tert-butyl piperazine-1-carboxylate (3.21 g, 17.21 mmol). The flask was sealed with a rubber septum, evacuated and backfilled nitrogen (this process was repeated a total of three times). Toluene (100 mL) was added. The reaction was stirred at 100° C. for 3 h. After cooling to room temperature, the mixture was concentrated. The residue was purified by silica gel column chromatography, eluted with 50% ethyl acetate in petroleum ether to give the desired product as a brown solid (5 g, 96%). LCMS calculated for C₁₇H₂₆N₃O₄ (M+H)⁺ m/z=336.2; found 336.1; ¹H NMR (400 MHz, CDCl₃) δ 7.99 (d, J=8.0 Hz, 1H), 7.33 (d, J=8.4 Hz, 1H), 3.99 (s, 3H), 3.63-3.61 (m, 4H), 2.96-2.94 (m, 4H), 2.67 (s, 3H), 1.49 (s, 9H).

### Step 3: Methyl 6-methyl-5-(piperazin-1-yl) picolinate

The solution of tert-butyl 4-(6-(methoxycarbonyl)-2-methylpyridin-3-yl)piperazine-1-carboxylate (5 g, 14.91 mmol) in hydrogen chloride (4 M in 1,4-dioxane, 50 mL) was stirred at room temperature for 1 h. The mixture was neutralized with saturated aqueous sodium bicarbonate. The resulting mixture was extracted with 25% isopropanol in chloroform (3×200 mL). The combined organic layers were washed with brine (2×300 mL), dried over anhydrous sodium sulfate. After filtration, the filtrate was concentrated under reduced pressure. The residue was purified by silica gel column chromatography, eluted with 10% methanol in dichloromethane to give the desired product as a brown oil (3.3 g, 94%). LCMS calculated for C₁₂H₁₈N₃O₂ (M+H)⁺ m/z=236.1; found 236.1; ¹H NMR (400 MHz, CDCl₃) δ 7.97 (d, J=8.4 Hz, 1H), 7.31 (d, J=8.4 Hz, 1H), 3.98 (s, 3H), 3.09-3.06 (m, 4H), 2.99-2.96 (m, 4H), 2.62 (s, 3H).

### Preparation of Provided Compounds

### Example 1: 5-(4-((3-Ethyl-2,4-dioxo-1,2,3,4-tetrahydroquinazolin-7-yl)methyl)piperazin-1-yl)-N-methylpicolinamide (I-1)

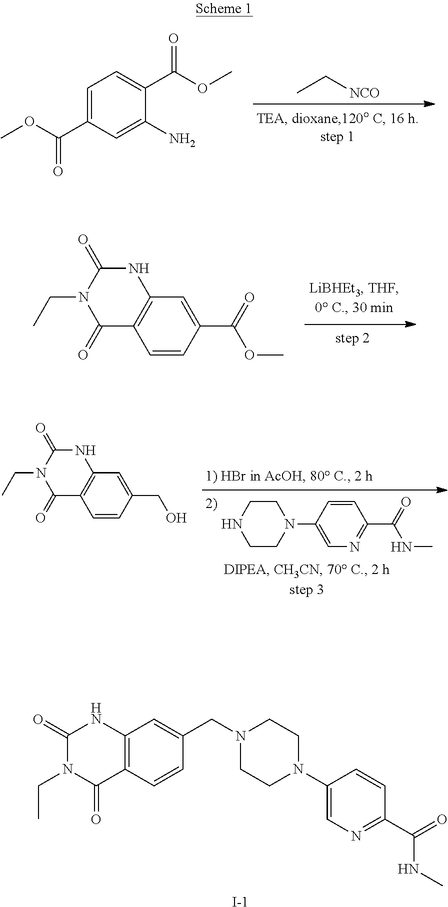

### Step 1: Methyl 3-ethyl-2,4-dioxo-1,2,3,4-tetrahydroquinazoline-7-carboxylate

To a mixture of dimethyl 2-aminoterephthalate (5 g, 23.9 mmol) in toluene (50 mL) were added isocyanatoethane (1.70 g, 23.9 mmol) and triethylamine (3.63 g, 35.9 mmol) at 0° C. under nitrogen atmosphere. The resulting mixture was stirred at 120° C. for 16 h. Upon cooling to room temperature, the crude product precipitated out; it was collected by filtration followed by washing with ethyl acetate (2×10 mL) and dried at room temperature to give the desired product as a white solid (4.8 g, 80%). LCMS calculated for C₁₂H₁₃N₂O₄ (M+H)⁺ m/z=249.1; found 249.2. ¹H NMR (400 MHz, CDCl₃) δ 8.53 (s, 1H), 8.21 (d, J=8.0 Hz, 1H), 7.85 (dd, J=8.0, 1.2 Hz, 1H), 7.71 (d, J=1.2 Hz, 1H), 4.15 (q, J=7.2 Hz, 2H), 3.98 (s, 3H), 1.32 (t, J=7.2 Hz, 3H).

### Step 2:3-Ethyl-7-(hydroxymethyl) quinazoline-2,4(1H,3H)-dione

To a mixture of methyl 3-ethyl-2,4-dioxo-1,2,3,4-tetrahydroquinazoline-7-carboxylate (100 mg, 0.4 mmol) in anhydrous tetrahydrofuran (10 mL) was added lithium triethylborohydride (1.0 M in THF, 1.0 mL, 1.0 mmol) dropwise at 0° C. under nitrogen atmosphere. The resulting mixture was stirred at the same temperature for 30 min, and then quenched by the addition of saturated aqueous solution of ammonium chloride; the mixture was extracted with dichloromethane (2×50 mL). The combined organics were dried with anhydrous sodium sulfate, filtered, and concentrated under reduced pressure. The residue was purified by reverse-phase flash chromatography (column: C18 silica gel; mobile phase: acetonitrile in water: elution: 5% to 50% gradient over 30 min; detector: UV 254 nm). The fractions were collected, combined and lyophilized to provide the desired product as a white solid (70 mg, 78%). LCMS calculated for C₁₁H₁₃N₂O₃ (M+H)⁺ m/z=221.1; found 221.3. ¹H NMR (400 MHz, DMSO-d₆) δ 11.38 (s, 1H), 7.87 (d, J=8.0 Hz, 1H), 7.17 (s, 1H), 7.10 (dd, J=8.0 Hz, 1H), 5.45 (t, J=5.2 Hz, 1H), 4.56 (d, J=5.2 Hz, 2H), 3.92 (q, J=7.2 Hz, 2H), 1.14 (t, J=7.2 Hz, 3H).

### Step 3: 5-(4-((3-Ethyl-2,4-dioxo-1,2,3,4-tetrahydroquinazolin-7-yl)methyl)piperazin-1-yl)-N-methylpicolinamide (I-1)

The mixture of 3-ethyl-7-(hydroxymethyl) quinazoline-2,4(1H,3H)-dione (50 mg, 0.24 mmol) was combined with hydrogen bromide (33 wt. % solution in glacial acid, 2 mL) at room temperature; the reaction was then heated at 80° C. under nitrogen atmosphere for 2 h. Upon cooling to room temperature, the reaction was concentrated under reduced pressure. The residue was taken in acetonitrile (3 mL) followed by addition of N-methyl-5-(piperazin-1-yl)picolinamide dihydrochloride (75 mg, 0.3 mmol) and N-ethyl-N-isopropylpropan-2-amine (235 mg, 1.8 mmol). Then the resulted mixture was heated at 70° C. for additional 2 h. The mixture was allowed to cool to room temperature and concentrated under reduced pressure. The residue was purified by prep-HPLC (column: Sunfire prep C₁₈ column, 30*150 mm, 5 um; mobile phase A: water (0.1% formic acid), mobile phase B: acetonitrile; flow rate: 60 mL/min; gradient: 8% B to 29% B over 7 min); eluted fractions were collected and lyophilized to provide the desired product as a light-yellow solid (33.8 mg). LCMS calculated for C₂₂H₂₇N₆O₃ (M+H)⁺ m/z=423.2; found 423.0. ¹H NMR (400 MHz, DMSO-d₆) δ 11.37 (s, 1H), 8.41-8.38 (m, 1H), 8.27 (d, J=2.8 Hz, 1H), 7.90 (d, J=8.4 Hz, 1H), 7.83 (d, J=8.4 Hz, 1H), 7.39 (dd, J=8.8, 2.8 Hz, 1H), 7.19-7.17 (m, 2H), 3.93 (q, J=7.2 Hz, 2H), 3.60 (s, 2H), 3.38-3.45 (m, 4H), 2.78 (d, J=5.2 Hz, 3H), 2.56-2.53 (m, 4H), 1.14 (t, J=7.2 Hz, 3H).

### Example 2: 5-(4-((3-Ethyl-2-oxo-1,2,3,4-tetrahydroquinazolin-7-yl)methyl)piperazin-1-yl)-N-methylpicolinamide (I-2)

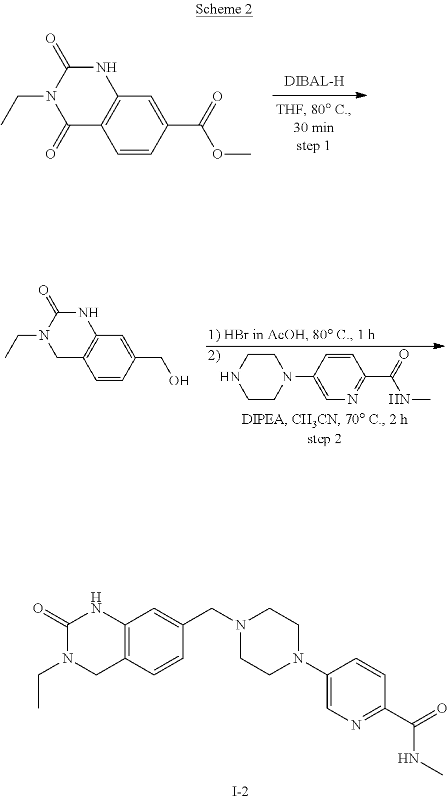

### Step 1:3-Ethyl-7-(hydroxymethyl)-3,4-dihydroquinazolin-2(1H)-one

To a mixture of methyl 3-ethyl-2,4-dioxo-1,2,3,4-tetrahydroquinazoline-7-carboxylate (2 g, 8.1 mmol) in anhydrous tetrahydrofuran (100 mL) was added diisobutylaluminium hydride solution (1.0 M in THF, 40.3 mL, 40.3 mmol) at 0° C. under nitrogen atmosphere. The resulting mixture was stirred at 80° C. for 30 min. Upon cooling to room temperature, the reaction was quenched by with saturated aqueous ammonium chloride solution at 0° C.; the mixture was extracted with dichloromethane (2×300 mL). The combined organics were dried with anhydrous sodium sulfate, filtered, and concentrated under reduced pressure. The residue was purified by silica gel column chromatography, eluted with ethyl acetate to provide the desired product as a white solid (800 mg, 48%). LCMS calculated for C₁₁H₁₅N₂O₂ (M+H)⁺ m/z=207.1; found 207.1. ¹H NMR (400 MHz, CDCl₃) δ 7.03-7.01 (m, 2H), 6.93-6.91 (m, 1H), 6.69 (s, 1H), 4.63 (s, 2H), 4.43 (s, 2H), 3.52-3.46 (m, 2H), 1.21 (t, J=7.2 Hz, 3H).

### Step 2: 5-(4-((3-Ethyl-2-oxo-1,2,3,4-tetrahydroquinazolin-7-yl)methyl)piperazin-1-yl)-N-methylpicolinamide (I-2)

The mixture of 3-ethyl-7-(hydroxymethyl)-1,4-dihydroquinazolin-2-one (50 mg, 0.2 mmol) was combined with hydrogen bromide (33 wt. % solution in glacial acid, 2 mL) at room temperature; the reaction was then heated at 80° C. under nitrogen atmosphere for 2 h. Upon cooling to room temperature, the reaction was concentrated under reduced pressure. The residue was taken in acetonitrile (3 mL) followed by addition of N-methyl-5-(piperazin-1-yl)picolinamide dihydrochloride (70 mg, 0.2 mmol) and N-ethyl-N-isopropylpropan-2-amine (235 mg, 1.8 mmol). Then the resulted mixture was heated at 70° C. for additional 2 h. The mixture was allowed to cool to room temperature and concentrated under reduced pressure. The residue was purified by prep-HPLC (column: Sunfire prep C₁₈ column, 30*150 mm, 5 um; mobile phase A: water (0.05% trifluoroacetic acid), mobile phase B: acetonitrile; flow rate: 60 mL/min; gradient: 5% B to 55% B over 7 min); eluted fractions were collected and lyophilized to provide the TFA salt of the desired product as a white solid (14.3 mg). LCMS calculated for C₂₂H₂₉N₆O₂ (M+H)⁺ m/z=409.2; found 409.0. ¹H NMR (400 MHz, DMSO-d₆+D₂O) δ 8.34 (d, J=2.8 Hz, 1H), 7.92 (d, J=8.8 Hz, 1H), 7.50 (dd, J=8.8, 2.8 Hz, 1H), 7.25 (d, J=7.2 Hz, 1H), 7.06 (dd, J=8.0, 1.6 Hz, 1H), 6.90 (s, 1H), 4.74 (s, 2H), 4.30 (s, 2H), 3.52-3.13 (m, 10H), 2.82 (s, 3H), 1.11 (t, J=7.2 Hz, 3H).

### Example 3: N-Methyl-5-(4-((2-oxo-1a,2,3,7b-tetrahydro-1H-cyclopropa[c]quinolin-5-yl)methyl)piperazin-1-yl)picolinamide (I-3)

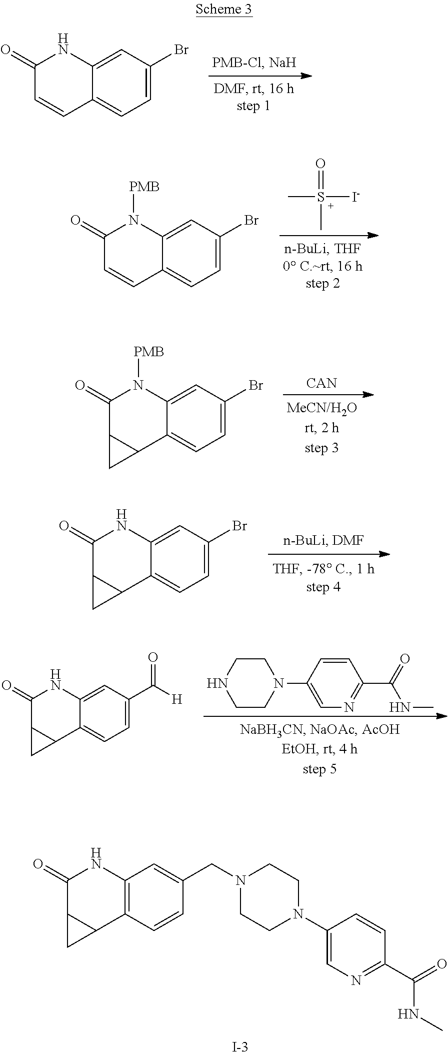

### Step 1: 7-Bromo-1-(4-methoxybenzyl) quinolin-2(1H)-one

To a mixture of 7-bromoquinolin-2(1H)-one (2 g, 8.9 mmol) in N,N-dimethylformamide (10 mL) was added sodium hydride (60%, 0.43 g, 10.7 mmol) in portions at 0° C. under nitrogen atmosphere. The mixture was stirred at the same temperature for 30 min, followed by the addition of 1-(chloromethyl)-4-methoxybenzene (2.1 g, 13.39 mmol) dropwise at 0° C. The resulting mixture was stirred at room temperature for 16 h and then quenched with saturated ammonium chloride solution; the mixture was extracted with ethyl acetate (2×50 mL). The combined organics were washed with brine (3×100 mL), dried with anhydrous sodium sulfate. After filtered, the filtrate was concentrated under reduced pressure. The residue was purified by silica gel column chromatography, eluted with 10% ethyl acetate in petroleum ether to provide the desired product as a white solid (1.6 g, 52%). LCMS calculated for C₁₇H₁₅BrNO₂ (M+H)⁺ m/z=344.0; found 344.0. ¹H NMR (400 MHz, CDCl₃) δ 7.67 (d, J=9.2 Hz, 1H), 7.49 (s, 1H), 7.40 (d, J=7.6 Hz, 1H), 7.31-7.28 (m, 1H), 7.19 (d, J=8.4 Hz, 2H), 6.87-6.84 (m, 2H), 6.79 (d, J=9.6 Hz, 1H), 5.43 (s, 2H), 3.77 (s, 3H).

### Step 2: 5-Bromo-3-(4-methoxybenzyl)-1,1a, 3,7b-tetrahydro-2H-cyclopropa[c]quinolin-2-one

To a mixture of iodotrimethyl-lambda6-sulfanone (5.11 g, 23.2 mmol) in anhydrous tetrahydrofuran (20 mL) was added n-butyllithium (2.5 M in hexanes, 9.30 mL, 23.2 mmol) dropwise at 0° C. under nitrogen atmosphere, followed by the addition of 7-bromo-1-[(4-methoxyphenyl)methyl]quinolin-2-one (1.6 g, 4.6 mmol) in anhydrous tetrahydrofuran (5 mL) dropwise at 0° C. The resulting mixture was stirred at room temperature for 16 h, and then quenched with water at 0° C.; the mixture was extracted with ethyl acetate (2×100 mL). The combined organics were dried with anhydrous sodium sulfate, filtered, and concentrated under reduced pressure. The residue was purified by silica gel column chromatography, eluted with 50% ethyl acetate in petroleum ether to provide the desired product as an off-white solid (1.1 g, 66%). LCMS calculated for C₁₈H₁₇BrNO₂ (M+H)⁺ m/z=358.0; found 358.0. ¹H NMR (400 MHz, CDCl₃) δ 7.20 (d, J=8.0 Hz, 1H), 7.13-7.07 (m, 3H), 7.01 (d, J=2.0 Hz, 1H), 6.86-6.83 (m, 2H), 5.21-5.17 (m, 1H), 4.96-4.92 (m, 1H), 3.77 (s, 3H), 2.55-2.50 (m, 1H), 2.43-2.38 (m, 1H), 1.67-1.62 (m, 1H), 0.68-0.64 (m, 1H).

### Step 3: 5-Bromo-1,1a, 3,7b-tetrahydro-2H-cyclopropa[c]quinolin-2-one

To a mixture of 5-bromo-3-(4-methoxybenzyl)-1,1a,3,7b-tetrahydro-2H-cyclopropa[c]quinolin-2-one (1 g, 2.8 mmol) in acetonitrile (4.5 mL) and water (0.5 mL) was added diammonium cerium (IV) nitrate (5.38 g, 9.8 mmol) in portions. The resulting mixture was stirred at room temperature for 2 h, and quenched with saturated aqueous solution of sodium carbonate; the mixture was extracted with ethyl acetate (2×100 mL). The combined organics were dried with anhydrous sodium sulfate, filtered, and concentrated under reduced pressure. The residue was purified by silica gel column chromatography, eluted with 20% ethyl acetate in petroleum ether to provide the desired product as a brown solid (400 mg, 60%). LCMS calculated for C₁₀H₉BrNO (M+H)⁺ m/z=238.0; found 238.0. ¹H NMR (400 MHz, CDCl₃) δ 8.89 (s, 1H), 7.21 (d, J=8.0 Hz, 1H), 7.11 (dd, J=8.0, 2.0 Hz, 1H), 6.97 (d, J=2.0 Hz, 1H), 2.53-2.48 (m, 1H), 2.21-2.15 (m, 1H), 1.70-1.64 (m, 1H), 0.84-0.70 (m, 1H).

### Step 4:2-Oxo-1a, 2,3,7b-tetrahydro-1H-cyclopropa[c]quinoline-5-carbaldehyde

To a mixture of 5-bromo-1,1a,3,7b-tetrahydro-2H-cyclopropa[c]quinolin-2-one (20 mg, 0.08 mmol) in anhydrous tetrahydrofuran (0.2 mL) was added n-butyllithium (2.5 M in hexanes, 0.12 mL, 0.3 mmol) at −78° C. under nitrogen atmosphere, followed by the addition of N,N-dimethylformamide (0.1 mL) at −78° C. The resulting mixture was warmed to room temperature and quenched with water; the mixture was extracted with ethyl acetate (2×10 mL). The combined organics were dried with anhydrous sodium sulfate, filtered, and concentrated under reduced pressure. The residue was purified by silica gel column chromatography, eluted with 10% ethyl acetate in petroleum ether to provide the desired product as a white solid (10 mg, 63%). LCMS calculated for C₁₁H₁₀NO₂ (M+H)⁺ m/z=188.1; found 188.0. ¹H NMR (400 MHz, CDCl₃) δ 9.94 (s, 1H), 8.69 (s, 1H), 7.53 (s, 2H), 7.29 (s, 1H), 2.64-2.59 (m, 1H), 2.30-2.24 (m, 1H), 1.80-1.75 (m, 1H), 0.89-0.79 (m, 1H).

### Step 5: N-methyl-5-(4-((2-oxo-1a,2,3,7b-tetrahydro-1H-cyclopropa[c]quinolin-5-yl)methyl)piperazin-1-yl)picolinamide (I-3)

The mixture of 2-oxo-1a,2,3,7b-tetrahydro-1H-cyclopropa[c]quinoline-5-carbaldehyde (30 mg, 0.2 mmol), N-methyl-5-(piperazin-1-yl)picolinamide dihydrochloride (47 mg, 0.2 mmol) and sodium acetate (26 mg, 0.3 mmol) in ethanol (3 mL) was stirred at room temperature for 20 min, followed by the addition of sodium cyanoborohydride (20 mg, 0.3 mmol) and acetic acid (19 mg, 0.3 mmol). The resulting mixture was stirred at room temperature for additional 4 h, then concentrated under vacuum and the residue was purified by reverse flash chromatography (column, C18 silica gel; mobile phase, acetonitrile in water, 5% to 95% gradient over 30 min). The fractions were collected, combined and lyophilized to provide the desired product as a white solid (9 mg). LCMS calculated for C₂₂H₂₆N₅O₂ (M+H)⁺ m/z=392.2; found 392.3. ¹H NMR (400 MHz, DMSO-d₆) δ 9.89 (s, 1H), 8.41-8.38 (m, 1H), 8.25 (d, J=2.8 Hz, 1H), 7.82 (d, J=8.8 Hz, 1H), 7.38 (dd, J=8.8, 2.8 Hz, 1H), 7.32 (d, J=8.0 Hz, 1H), 6.89-6.85 (m, 2H), 3.43 (s, 2H), 3.33-3.30 (m, 4H), 2.78 (d, J=4.8 Hz, 3H), 2.54-2.52 (m, 4H), 2.50-2.43 (m, 1H), 2.08-1.95 (m, 1H), 1.59-1.54 (m, 1H), 0.52-0.49 (m, 1H).

### Example 4: N-methyl-5-(4-((4-oxo-2,3,4,5-tetrahydro-1H-cyclopenta[c]quinolin-7-yl)methyl)piperazin-1-yl)picolinamide (I-4)

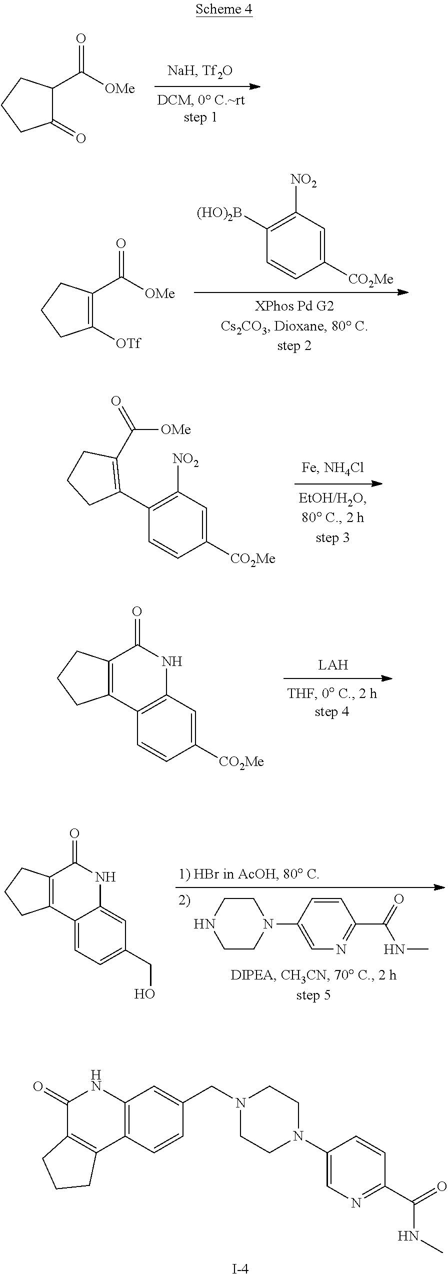

### Step 1: Methyl 2-(((trifluoromethyl) sulfonyl)oxy)cyclopent-1-ene-1-carboxylate

To a mixture of methyl 2-oxocyclopentane-1-carboxylate (5 g, 35.2 mmol) in dichloromethane (50 mL) was added sodium hydride (60%, 1.69 g, 42.2 mmol) in portions at 0° C. under nitrogen atmosphere. The mixture was stirred at the same temperature for 30 min, followed by the addition of trifluoromethanesulfonic anhydride (11.91 g, 42.2 mmol) dropwise at 0° C. The resulting mixture was stirred at room temperature for 16 h and then quenched with water; the mixture was extracted with dichloromethane (3×200 mL). The combined organics were dried with anhydrous sodium sulfate, filtered, and concentrated under reduced pressure. The residue was purified by silica gel column chromatography, eluted with 20% ethyl acetate in petroleum ether to provide the desired product as a colorless oil (8 g, 83%). ¹H NMR (300 MHz, CDCl₃) δ 3.81 (s, 3H), 2.81-2.69 (m, 4H), 2.09-1.99 (m, 2H).

### Step 2: Methyl 4-(2-(methoxycarbonyl)cyclopent-1-en-1-yl)-3-nitrobenzoate

The mixture of methyl 2-(((trifluoromethyl) sulfonyl)oxy)cyclopent-1-ene-1-carboxylate (500 mg, 1.8 mmol), 4-(methoxycarbonyl)-2-nitrophenylboronic acid (492 mg, 2.188 mmol), cesium carbonate (1188 mg, 3.6 mmol) and chloro(2-dicyclohexylphosphino-2′,4′,6′-triisoporpyl-1,1′-biphenyl)[2-(2′-amino-1,1′-biphenyl)]palladium(II) (143 mg, 0.2 mmol) in 1,4-dioxane (10 mL) was stirred at 80° C. for 16 h under nitrogen atmosphere. Upon cooling to room temperature, the mixture was concentrated under reduced pressure. The residue was purified by silica gel column chromatography, eluted with 20% ethyl acetate in petroleum ether to provide the desired product as a white solid (500 mg, 90%). ¹H NMR (400 MHz, CDCl₃) δ 8.70 (d, J=1.6 Hz, 1H), 8.24 (dd, J=8.0, 1.6 Hz, 1H), 7.31 (d, J=8.0 Hz, 1H), 3.98 (s, 3H), 3.50 (s, 3H), 2.91-2.79 (m, 4H), 2.17-2.09 (m, 2H).

### Step 3: Methyl 4-oxo-2, 3, 4,5-tetrahydro-1H-cyclopenta[c]quinoline-7-carboxylate

To a mixture of methyl 4-(2-(methoxycarbonyl)cyclopent-1-en-1-yl)-3-nitrobenzoate (500 mg, 1.6 mmol) in ethanol (15 mL) and water (2.5 mL) were added iron (457 mg, 8.2 mmol) and ammonium chloride (263 mg, 4.9 mmol). The resulting mixture was stirred at 80° C. for 2 h. Upon cooling to room temperature, the mixture was concentrated under reduced pressure. The residue was purified by silica gel column chromatography, eluted with 50% ethyl acetate to provide the desired product as a white solid (250 mg, 63%). LCMS calculated for C₁₄H₁₄NO₃ (M+H)⁺ m/z=244.1; found 244.1. ¹H NMR (400 MHz, CDCl₃) δ 9.41 (brs, 1H), 8.87-7.80 (m, 2H), 7.59 (d, J=8.0 Hz, 1H), 3.97 (s, 3H), 3.25-3.13 (m, 4H), 2.29-2.23 (m, 2H).

### Step 4: 7-(Hydroxymethyl)-1,2,3,5-tetrahydro-4H-cyclopenta[c]quinolin-4-one

To a mixture of methyl 4-oxo-2,3,4,5-tetrahydro-1H-cyclopenta[c]quinoline-7-carboxylate (200 mg, 0.8 mmol) in anhydrous tetrahydrofuran (8 mL) was added lithium aluminum hydride (2.0 M in THF, 0.8 mL, 1.6 mmol) dropwise at 0° C. under nitrogen atmosphere. The resulting mixture was stirred at the same temperature for 1.5 h, and then quenched with water (one drop), 15% sodium hydroxide (one drop) and water (three drops) at 0° C., followed by the addition of anhydrous sodium sulfate (1 g). The mixture was stirred for 10 min at room temperature, filtered, and concentrated under vacuum. The residue was purified by silica gel column chromatography, eluted with 10% methanol in dichloromethane to provide the desired product as a white solid (50 mg, 28%). LCMS calculated for C₁₃H₁₄NO₂ (M+H)⁺ m/z=216.1; found 216.1.

### Step 5: N-methyl-5-(4-((4-oxo-2,3,4,5-tetrahydro-1H-cyclopenta[c]quinolin-7-yl)methyl)piperazin-1-yl)picolinamide (I-4)

The mixture of 7-(hydroxymethyl)-1,2,3,5-tetrahydro-4H-cyclopenta[c]quinolin-4-one (50 mg, 0.2 mmol) was combined with hydrogen bromide (33 wt. % solution in glacial acid, 2 mL) at room temperature; the reaction was then heated at 80° C. under nitrogen atmosphere for 2 h. Upon cooling to room temperature, the reaction was concentrated under reduced pressure. The residue was taken in acetonitrile (3 mL) followed by addition of N-methyl-5-(piperazin-1-yl)picolinamide dihydrochloride (68 mg, 0.2 mmol) and N-ethyl-N-isopropylpropan-2-amine (235 mg, 1.8 mmol). Then the resulted mixture was heated at 70° C. for additional 2 h. The mixture was allowed to cool to room temperature and concentrated under reduced pressure. The residue was purified by prep-HPLC (Column: Xselect CSH C₁₈ OBD Column 30*150 mm 5 um; mobile phase A: water (0.05% trifluoroacetic acid), mobile Phase B: acetonitrile; flow rate: 60 mL/min; gradient: 10% B to 22% B over 7 min); eluted fractions were collected and lyophilized. The residue was re-purified by prep-HPLC (column: XBridge Prep OBD C18 Column, 30*150 mm, 5 um; mobile phase A: water (10 mmol/L ammonium bicarbonate), mobile phase B: acetonitrile; flow rate: 60 mL/min). The fractions were collected, combined and lyophilized to provide the desired product as a white solid (2 mg).

LCMS calculated for C₂₄H₂₈N₅O₂ (M+H)⁺ m/z=418.2; found 418.3. ¹H NMR (400 MHz, DMSO-d₆) δ 11.54 (s, 1H), 8.41-8.38 (m, 1H), 8.26 (d, J=2.8 Hz, 1H), 7.82 (d, J=8.8 Hz, 1H), 7.50 (d, J=8.0 Hz, 1H), 7.39 (dd, J=9.2, 2.4 Hz, 1H), 7.33 (s, 1H), 7.17 (d, J=8.0 Hz, 1H), 3.60 (s, 2H), 3.33-3.29 (m, 4H), 3.08 (t, J=7.6 Hz, 2H), 2.78-2.74 (m, 5H), 2.55-2.53 (m, 4H), 2.14-2.06 (m, 2H).

### Example 5: N-methyl-5-(4-((2′-oxo-1′,4′-dihydro-2′H-spiro[cyclopropane-1,3′-quinolin]-7′-yl)methyl)piperazin-1-yl)picolinamide (I-5)

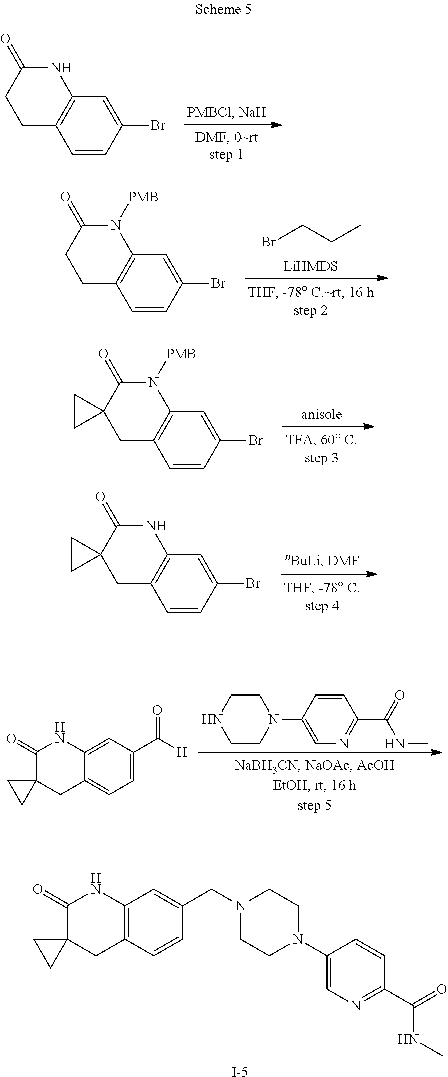

### Step 1: 7-Bromo-1-(4-methoxybenzyl)-3,4-dihydroquinolin-2(1H)-one

To a mixture of 7-bromo-3,4-dihydroquinolin-2(1H)-one (2.5 g, 11.1 mmol) in N,N-dimethylformamide (20 mL) was added sodium hydride (60%, 0.4 g, 16.6 mmol) in portions at 0° C. under the nitrogen atmosphere. The mixture was stirred at the same temperature for 30 min, followed by the addition of 1-(chloromethyl)-4-methoxybenzene (1.91 g, 12.2 mmol). The resulting mixture was stirred at room temperature for 16 h and then quenched with saturated ammonium chloride solution; the mixture was extracted with ethyl acetate (3×100 mL). The combined organics were washed with brine (3×100 mL), dried with anhydrous sodium sulfate. After filtered, the filtrate was concentrated under reduced pressure. The residue was purified by silica gel column chromatography, eluted with 20% ethyl acetate in petroleum ether to provide the desired product as a yellow solid (3.6 g, 94%). LCMS calculated for C₁₇H17BrNO₂ (M+H)⁺ m/z=346.0; found 346.1. ¹H NMR (400 MHz, DMSO-d₆) δ 7.22-7.10 (m, 5H), 6.94-6.85 (m, 2H), 5.10 (s, 2H), 3.73 (s, 3H), 2.93-2.82 (m, 2H), 2.74-2.66 (m, 2H).

### Step 2: 7′-Bromo-1′-(4-methoxybenzyl)-1′,4′-dihydro-2′H-spiro[cyclopropane-1,3′-quinolin]-2′-one

To a mixture of 7-bromo-1-(4-methoxybenzyl)-3,4-dihydroquinolin-2(1H)-one (3.6 g, 10.4 mmol) in anhydrous tetrahydrofuran (20 mL) was added lithium bis(trimethylsilyl)amide (1.0 M in THF, 12.5 mL, 12.5 mmol) at −78° C. under nitrogen atmosphere. The mixture was stirred at the same temperature for 1 h, followed by the addition of 1-bromo-2-chloroethane (4.47 g, 31.2 mmol). The resulting mixture was stirred at room temperature for 16 h, and then quenched with saturated aqueous ammonium chloride solution; the mixture was extracted with ethyl acetate (2×100 mL). The combined organics were dried with anhydrous sodium sulfate, filtered, and concentrated under reduced pressure. The residue was purified by reverse flash (column, C18 silica gel; mobile phase, acetonitrile in water, elution: 0% to 50% gradient over 35 min). The fractions were collected, combined and lyophilized to provide the desired product as a white solid (560 mg, 14%). LCMS calculated for C₁₉H₁₉BrNO₂ (M+H)⁺ m/z=372.1; found 371.9. ¹H NMR (400 MHz, CDCl₃) δ 7.13-7.08 (m, 3H), 7.04 (d, J=2.0 Hz, 1H), 6.94 (d, J=8.0 Hz, 1H), 6.86-6.83 (m, 2H), 5.07 (s, 2H), 3.78 (s, 3H), 2.80 (s, 2H), 1.38-1.35 (m, 2H), 0.78-0.75 (m, 2H).

### Step 3: 7′-Bromo-1′,4′-dihydro-2′H-spiro[cyclopropane-1,3′-quinolin]-2′-one

The mixture of 7′-bromo-1′-(4-methoxybenzyl)-1′,4′-dihydro-2′H-spiro[cyclopropane-1,3′-quinolin]-2′-one (520 mg, 1.4 mmol) and anisole (151 mg, 1.4 mmol) in trifluoroacetic acid (3 mL) was stirred at 60° C. for 3 h. Upon cooling to room temperature, the mixture was concentrated under vacuum. The residue was taken in ethyl acetate (100 mL) and washed with saturated aqueous sodium bicarbonate solution (3×50 mL). The combined organics were dried with anhydrous sodium sulfate, filtered, and concentrated under reduced pressure. The residue was purified by silica gel column chromatography, eluted with 50% petroleum ether in ethyl acetate to provide the desired product as a yellow solid (230 mg, 65%). LCMS calculated for C₁₁H₁₁BrNO (M+H)⁺ m/z=252.0; found 252.1. ¹H NMR (400 MHz, CDCl₃) δ 8.79 (s, 1H), 7.11 (dd, J=8.0, 1.6 Hz, 1H), 6.96-6.94 (m, 2H), 2.83 (s, 2H), 1.41-1.38 (m, 2H), 0.81-0.78 (m, 2H).

### Step 4: 2′-Oxo-1′,4′-dihydro-2′H-spiro[cyclopropane-1,3′-quinoline]-7′-carbaldehyde

To a mixture of 7′-bromo-1′,4′-dihydro-2′H-spiro[cyclopropane-1,3′-quinolin]-2′-one (100 mg, 0.4 mmol) in anhydrous tetrahydrofuran (3 mL) was added n-butyllithium (2.5 M in hexanes, 0.48 mL, 0.1 mmol) at −78° C. under nitrogen atmosphere. The resulting mixture was stirred at the same temperature for 1 h, followed by the addition of N,N-dimethylformamide (144 mg, 2.0 mmol). The resulting mixture was warmed to room temperature and quenched with water; the mixture was extracted with ethyl acetate (3×50 mL). The combined organics were dried with anhydrous sodium sulfate, filtered, and concentrated under reduced pressure. The residue was purified by silica gel column chromatography, eluted with 50% ethyl acetate in petroleum ether to provide the desired product as a yellow solid (40 mg, 50%). LCMS calculated for C₁₂H₁₂NO₂ (M+H)⁺ m/z=202.1; found 202.0. ¹H NMR (400 MHz, DMSO-d₆) δ 10.43 (s, 1H), 9.92 (s, 1H), 7.52 (dd, J=7.6, 1.6 Hz, 1H), 7.39-7.35 (m, 2H), 2.95 (s, 2H), 1.12-1.05 (m, 2H), 0.78-0.75 (m, 2H).

### Step 5: N-methyl-5-(4-((2′-oxo-1′,4′-dihydro-2′H-spiro[cyclopropane-1,3′-quinolin]-7′-yl)methyl)piperazin-1-yl)picolinamide (I-5)

The mixture of 2′-oxo-1′,4′-dihydro-2′H-spiro[cyclopropane-1,3′-quinoline]-7′-carbaldehyde (40 mg, 0.2 mmol), N-methyl-5-(piperazin-1-yl)picolinamide dihydrochloride (58 mg, 0.2 mmol) and sodium acetate (32 mg, 0.4 mmol) in ethanol (1 mL) was stirred for 20 min, followed by the addition of acetic acid (24 mg, 0.4 mmol) and sodium cyanoborohydride (25 mg, 0.4 mmol). The resulting mixture was stirred for 16 h, and then concentrated under reduced pressure and the residue was purified by reverse flash (column, C18 silica gel; mobile phase, acetonitrile in water, 0%-50% gradient over 35 min). The fractions were collected, combined and lyophilized to provide the desired product as a white solid (8.1 mg). LCMS calculated for C₂₃H₂₈N₅O₂ (M+H)⁺ m/z=406.2; found 406.1. ¹H NMR (300 MHz, DMSO-d₆) δ 10.11 (s, 1H), 8.43-8.38 (m, 1H), 8.26 (d, J=2.7 Hz, 1H), 7.83 (d, J=8.7 Hz, 1H), 7.38 (dd, J=9.0, 3.0 Hz, 1H), 7.05 (d, J=7.8 Hz, 1H), 6.90-6.87 (m, 2H), 3.45 (s, 2H), 3.34-3.31 (m, 4H), 2.81-2.78 (m, 5H), 2.55-2.52 (m, 4H), 1.09-1.06 (m, 2H), 0.73-0.69 (m, 2H).

### Example 6: N-methyl-5-(4-((6-oxo-6,7,8,9-tetrahydro-5H-cyclopenta[c][1,5]naphthyridin-3-yl)methyl)piperazin-1-yl)picolinamide (I-6)

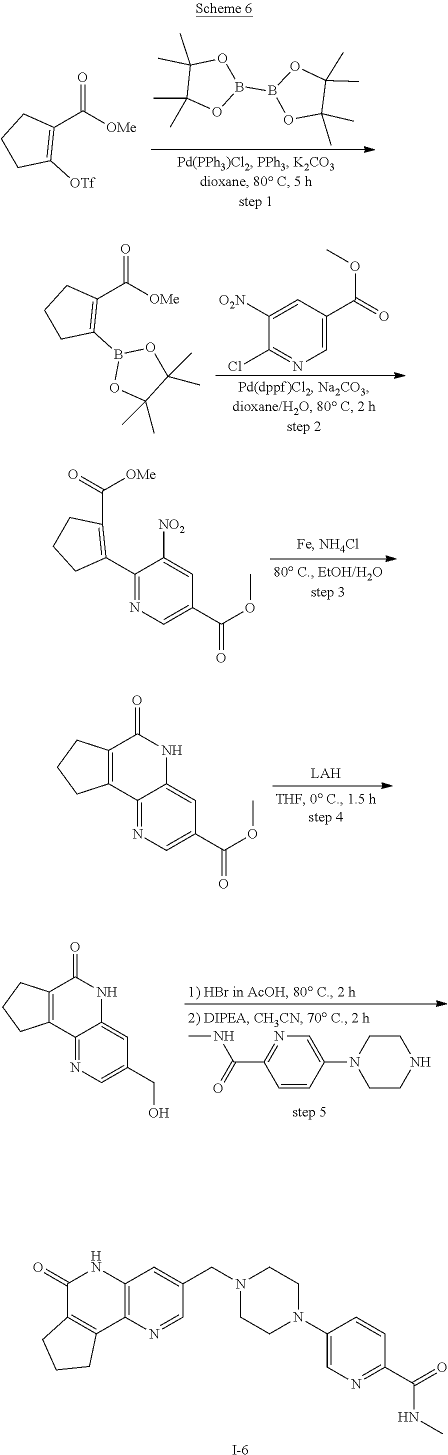

### Step 1: Methyl 2-(4,4,5,5-tetramethyl-1,3,2-dioxaborolan-2-yl)cyclopent-1-ene-1-carboxylate

The mixture of bis(pinacolato)diboron (2.04 g, 8.0 mmol), bis(triphenylphosphine)palladium(II) chloride (0.15 g, 0.2 mmol), triphenylphosphine (0.11 g, 0.4 mmol), methyl 2-(((trifluoromethyl) sulfonyl)oxy)cyclopent-1-ene-1-carboxylate (2 g, 7.3 mmol) and potassium carbonate (1.51 g, 10.9 mmol) in 1,4-dioxane (20 mL) was heated at 80° C. for 5 h under nitrogen atmosphere. Upon cooling to room temperature, the mixture was diluted with ethyl acetate (100 mL). The organic layers were washed with brine (3×50 mL), dried with anhydrous sodium sulfate, filtered, and concentrated under reduced pressure. The residue was purified by silica gel column chromatography, eluted with 40% ethyl acetate in petroleum ether to provide the desired product as a white solid (1.3 g, 71%). LCMS calculated for C₁₃H₂₂BO₄ (M+H)⁺ m/z=253.2; found 253.0.

### Step 2: Methyl 6-(2-(methoxycarbonyl)cyclopent-1-en-1-yl)-5-nitronicotinate

The mixture of methyl 6-chloro-5-nitropyridine-3-carboxylate (600 mg, 2.8 mmol), methyl 2-(4,4,5,5-tetramethyl-1,3,2-dioxaborolan-2-yl)cyclopent-1-ene-1-carboxylate (1.05 g, 4.2 mmol), sodium carbonate (587 mg, 5.5 mmol) and [1,1′-bis(diphenylphosphino)ferrocene]dichloropalladium(II) (225 mg, 0.3 mmol) in 1,4-dioxane (7 mL) and water (1 mL) was heated at 80° C. for 2 h. Upon cooling to room temperature, the mixture was diluted with ethyl acetate (100 mL). The organic layers were washed with brine (3×50 mL), dried with anhydrous sodium sulfate, filtered, and concentrated under reduced pressure. The residue was purified by silica gel column chromatography, eluted with 5% methanol in dichloromethane to provide the desired product as a yellow solid (77 mg, 11%). LCMS calculated for C₁₄H₁₅N₂O₆ (M+H)⁺ m/z=307.1; found 307.0.

### Step 3: methyl 6-oxo-6,7,8,9-tetrahydro-5H-cyclopenta[c][1,5]naphthyridine-3-carboxylate

The mixture of methyl 6-(2-(methoxycarbonyl)cyclopent-1-en-1-yl)-5-nitronicotinate (77 mg, 0.3 mmol), iron (70 mg, 1.3 mmol) and ammonium chloride (40 mg, 0.7 mmol) in ethanol (5 mL) and water (1 mL) was heated at 80° C. for 1.5 h. Upon cooling to room temperature, the mixture was concentrated under reduced pressure. The residue was purified by silica gel column chromatography, eluted with 10% methanol in dichloromethane to provide the desired product as a yellow solid (44 mg, 72%). LCMS calculated for C₁₃H₁₃N₂O₃ (M+H)⁺ m/z=245.1; found 245.0.

### Step 4:3-(Hydroxymethyl)-5,7,8,9-tetrahydro-6H-cyclopenta[c][1,5]naphthyridin-6-one

To a mixture of methyl 6-oxo-6,7,8,9-tetrahydro-5H-cyclopenta[c][1,5]naphthyridine-3-carboxylate (44 mg, 0.2 mmol) in anhydrous tetrahydrofuran (2.5 mL) was added lithium aluminum hydride (2.0 M in THF, 0.15 mL, 0.3 mmol) dropwise at 0° C. under nitrogen atmosphere. The resulting mixture was stirred at the same temperature for 1.5 h, and then quenched with water (one drop), 15% sodium hydroxide (one drop) and water (three drops) at 0° C., followed by the addition of anhydrous sodium sulfate (0.3 g). The mixture was stirred for 10 min at room temperature, filtered, and concentrated under vacuum. The residue was purified by reverse phase flash chromatography (column, C18 silica gel; mobile phase, methanol in water (10 mM ammonium bicarbonate), 10% to 70% gradient over 20 min). The fractions were collected, combined and lyophilized to provide the desired product as a light-yellow solid (17 mg, 44%). LCMS calculated for C₁₂H₁₃N₂O₂ (M+H)⁺ m/z=217.1; found 217.0.

### Step 5: N-methyl-5-(4-((6-oxo-6,7,8,9-tetrahydro-5H-cyclopenta[c][1,5]naphthyridin-3-yl)methyl)piperazin-1-yl)picolinamide (I-6)

The mixture of 3-(hydroxymethyl)-5,7,8,9-tetrahydro-6H-cyclopenta[c][1,5]naphthyridin-6-one (17 mg, 0.1 mmol) was combined with hydrogen bromide (33 wt. % solution in glacial acid, 0.3 mL) at room temperature; the reaction was then heated at 80° C. under nitrogen atmosphere for 2 h. Upon cooling to room temperature, the reaction was concentrated under reduced pressure. The residue was taken in acetonitrile (0.3 mL), followed by addition of N-methyl-5-(piperazin-1-yl)pyridine-2-carboxamide dihydrochloride (24 mg, 0.1 mmol) and N-ethyl-N-isopropylpropan-2-amine (102 mg, 0.8 mmol). Then the resulted mixture was heated at 70° C. for additional 2 h. The mixture was allowed to cool to room temperature and concentrated under reduced pressure. The residue was purified by prep-HPLC (column: Sunfire prep C18 column, 30*150 mm, 5 um; mobile phase A: water (0.1% formic acid), mobile phase B: acetonitrile; flow rate: 60 mL/min; Gradient: 8% B over 17% B in 7 min); eluted fractions were collected and lyophilized to provide the formate salt of the desired product as a white solid (6.4 mg). LCMS calculated for C₂₃H₂₇N₆O₂ (M+H)⁺ m/z=419.2; found 419.1; ¹H NMR (300 MHz, DMSO-d₆) δ 11.70 (s, 1H), 8.43-8.37 (m, 2H), 8.28-8.25 (m, 1H), 7.83 (d, J=8.7 Hz, 1H), 7.67 (d, J=1.8 Hz, 1H), 7.39 (dd, J=9.0, 3.0 Hz, 1H), 3.66 (s, 2H), 3.36-3.33 (m, 4H), 3.18 (t, J=7.5 Hz, 2H), 2.84-2.78 (m, 5H), 2.58-2.55 (m, 4H), 2.17-2.07 (m, 2H).

### Example 7: N-methyl-5-(4-((3-methyl-4-oxo-4,5-dihydro-3H-pyrrolo[2,3-c]quinolin-7-yl)methyl)piperazin-1-yl)picolinamide (I-7)

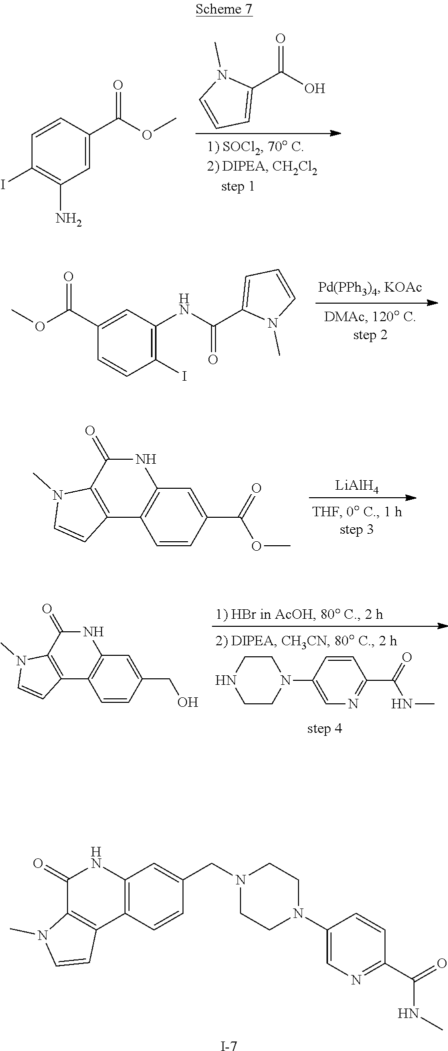

### Step 1: Methyl 4-iodo-3-(1-methyl-1H-pyrrole-2-carboxamido)benzoate

The mixture of 1-methylpyrrole-2-carboxylic acid (6.01 g, 48 mmol) in thionyl chloride (18 mL) and toluene (54 mL) was heated at 70° C. for 2 h. Upon cooling to room temperature, the mixture was concentrated under reduced pressure. The residue was taken in dichloromethane (60 mL), followed by the addition of methyl 3-amino-4-iodobenzoate (13.3 g, 48 mmol) in dichloromethane (120 mL) and N-ethyl-N-isopropylpropan-2-amine (18.61 g, 144 mmol) at 0° C. The mixture was stirred at room temperature for 16 h, and then concentrated under reduced pressure. The residue was purified by silica gel column chromatography, eluted with 50% ethyl acetate in petroleum ether to provide the desired product as a yellow solid (3.8 g, 20%). LCMS calculated for C₁₄H₁₄ IN₂O₃ (M+H)⁺ m/z=385.0; found 385.1. ¹H NMR (400 MHz, CDCl₃) δ 8.93 (d, J=2.0 Hz, 1H), 8.08 (s, 1H), 7.89 (d, J=8.4 Hz, 1H), 7.50 (dd, J=8.4, 2.0 Hz, 1H), 6.89 (dd, J=4.0, 1.6 Hz, 1H), 6.86-6.83 (m, 1H), 6.19 (dd, J=4.0, 2.45 Hz, 1H), 4.01 (s, 3H), 3.92 (s, 3H).

### Step 2: methyl 3-methyl-4-oxo-4,5-dihydro-3H-pyrrolo[2,3-c]quinoline-7-carboxylate

The mixture of methyl 4-iodo-3-(1-methylpyrrole-2-amido)benzoate (3.8 g, 9.9 mmol), tetrakis(triphenylphosphine)palladium (1.14 g, 1 mmol) and potassium acetate (1.55 g, 15.8 mmol) in N,N-dimethylacetamide (76 mL) was heated at 120° C. for 16 h under nitrogen atmosphere. Upon cooling to room temperature, the mixture was diluted with water (150 mL), and then extracted with ethyl acetate (3×150 mL). The combined organic layers were washed with brine (300 mL), dried with anhydrous sodium sulfate. After filtration, the filtrate was concentrated under reduced pressure. The residue was purified by reverse phase flash chromatography (column: WelFlash TM C18-I, 20-40 um, 120 g; mobile phase: methanol in water (0.1% formic acid); 10% to 45% gradient over 20 min). The fractions were collected, combined and lyophilized to provide the desired product as a white solid (220 mg, 8%). LCMS calculated for C₁₄H₁₃N₂O₃ (M+H)⁺ m/z=257.1; found 257.2. ¹H NMR (300 MHz, CDCl₃) δ 9.24 (s, 1H), 7.95 (d, J=1.2 Hz, 1H), 7.93 (d, J=1.2 Hz, 2H), 7.16 (d, J=3.0 Hz, 1H), 6.81 (d, J=3.0 Hz, 1H), 4.26 (s, 3H), 3.99 (s, 3H).

### Step 3: 7-(Hydroxymethyl)-3-methyl-5H-pyrrolo[2,3-c]quinolin-4-one

To a mixture of methyl 3-methyl-4-oxo-5H-pyrrolo[2,3-c]quinoline-7-carboxylate (250 mg, 0.98 mmol) in anhydrous tetrahydrofuran (5 mL) was added lithium aluminum hydride (2.0 M in THF, 0.73 mL, 1.46 mmol) at 0° C. under nitrogen atmosphere. The mixture was stirred at the same temperature for additional 2 h, and then quenched with water (one drop), 15% sodium hydroxide (one drop) and water (three drops) at 0° C., followed by the addition of anhydrous sodium sulfate (1 g). The mixture was stirred for 10 min at room temperature, filtered, and concentrated under vacuum. The residue was purified by reverse flash chromatography (column: WelFlash™ C18-I, 20-40 um, 330 g; mobile phase, methanol in water (10 mM ammonium bicarbonate), 20% to 45% gradient over 20 min). The fractions were collected, combined and lyophilized to provide the desired product as a white solid (150 mg, 67%). LCMS calculated for C₁₃H₁₃N₂O₂ (M+H)⁺ m/z=229.1; found 229.3; ¹H NMR (300 MHz, DMSO-d₆) δ 11.28 (s, 1H), 7.84 (d, J=8.1 Hz, 1H), 7.36 (d, J=2.7 Hz, 1H), 7.32 (s, 1H), 7.10 (d, J=8.1 Hz, 1H), 6.80 (d, J=2.7 Hz, 1H), 5.24 (t, J=5.7 Hz, 1H), 4.54 (d, J=5.7 Hz, 2H), 4.10 (s, 3H).

### Step 4: N-methyl-5-(4-((3-methyl-4-oxo-4,5-dihydro-3H-pyrrolo[2,3-c]quinolin-7-yl)methyl)piperazin-1-yl)picolinamide (I-7)

The mixture of 7-(hydroxymethyl)-3-methyl-5H-pyrrolo[2,3-c]quinolin-4-one (60 mg, 0.3 mmol) was combined with hydrogen bromide (33 wt. % solution in glacial acid, 2 mL) at room temperature; the reaction was then heated at 80° C. under nitrogen atmosphere for 2 h. Upon cooling to room temperature, the reaction was concentrated under reduced pressure. The residue was taken in 1-methylpyrrolidin-2-one (2 mL) followed by addition of N-methyl-5-(piperazin-1-yl)picolinamide dihydrochloride (84 mg, 0.3 mmol) and N-ethyl-N-isopropylpropan-2-amine (201 mg, 1.6 mmol). Then the resulted mixture was heated at 80° C. for additional 2 h. The mixture was allowed to cool to room temperature. The mixture was purified by prep-HPLC (column: YMC-Actus Triart C₁₈ ExRS, 30*150 mm, 5 um; mobile phase A: water (10 mM ammonium bicarbonate), mobile phase B: methanol; flow rate: 60 mL/min; gradient: 50% B to 80% B over 7 min); eluted fractions were collected and lyophilized to provide the desired product as a white solid (41.3 mg). LCMS calculated for C₂₄H₂₇N₆O₂ (M+H)⁺ m/z=431.2; found 431.2; ¹H NMR (300 MHz, DMSO-d₆) δ 11.26 (s, 1H), 8.41 (d, J=5.1 Hz, 1H), 8.27 (d, J=3.0 Hz, 1H), 7.91-7.77 (m, 2H), 7.39 (dd, J=11.4, 2.7 Hz, 2H), 7.33 (d, J=1.5 Hz, 1H), 7.14 (dd, J=7.8, 1.5 Hz, 1H), 6.81 (d, J=2.7 Hz, 1H), 4.10 (s, 3H), 3.58 (s, 2H), 3.38-3.35 (m, 2H), 3.31-3.29 (m, 2H), 2.78 (d, J=4.8 Hz, 3H), 2.54 (d, J=6.0 Hz, 4H).

### Example 8: N-methyl-5-(4-((1-methyl-4-oxo-4,5-dihydro-1H-pyrrolo[3,2-c]quinolin-7-yl)methyl)piperazin-1-yl)picolinamide (I-8)

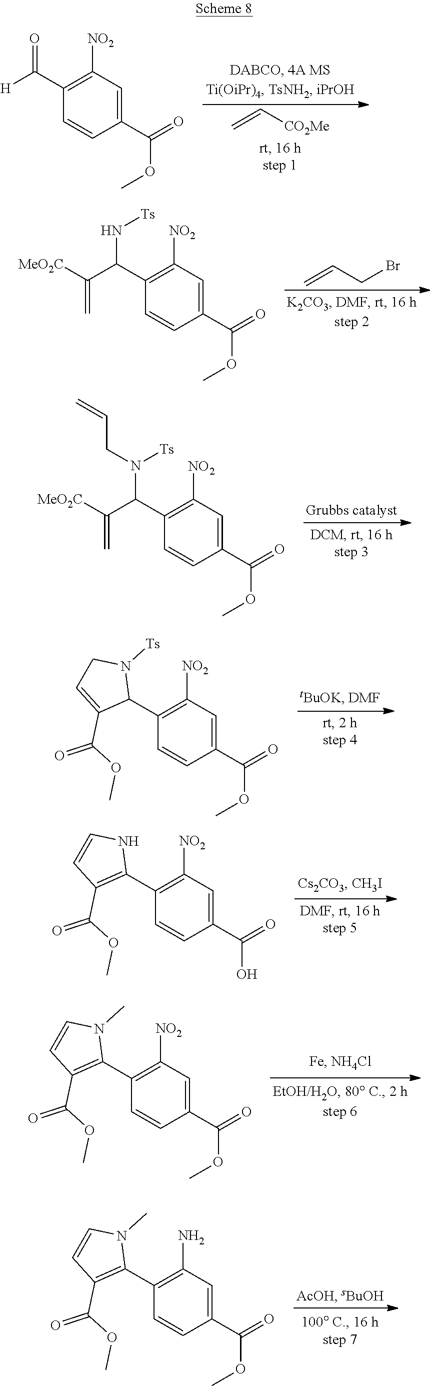

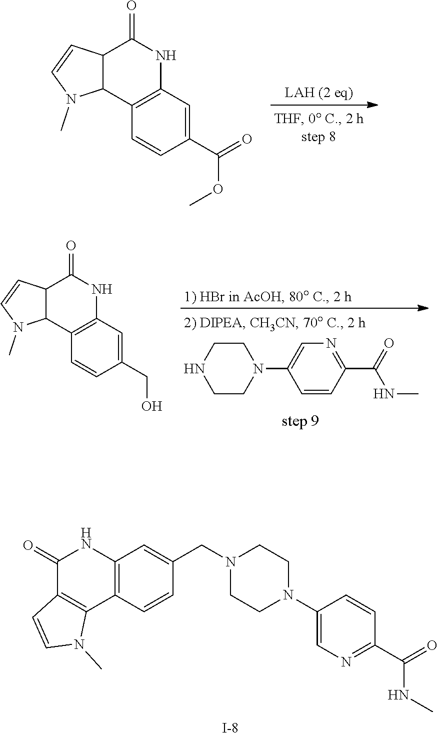

### Step 1: Methyl 4-(2-(methoxycarbonyl)-1-((4-methylphenyl) sulfonamido) allyl)-3-nitrobenzoate

To a mixture of 4-methylbenzenesulfonamide (9 g, 52.6 mmol), 1,4-diazabicyclo[2,2,2]octane (0.88 g, 7.9 mmol) and molecular sieves (4 Å, 10.5 g) in isopropanol (27 mL) were added methyl 4-formyl-3-nitrobenzoate (15.94 g, 76.2 mmol), methyl acrylate (6.79 g, 78.9 mmol) and titanium tetraisopropanolate (0.3 g, 1.1 mmol). The resulting mixture was stirred at room temperature for 16 h, and then filtered over Celite. The Celite was rinsed with dichloromethane (3×30 mL). The solvent was evaporated and the residue was dissolved in ethyl acetate (1000 mL), neutralized with aqueous potassium bisulfate (10%), washed with saturated aqueous sodium bicarbonate (300 mL) and brine (300 mL). The organics were dried with anhydrous sodium sulfate, filtered, and concentrated under reduced pressure. The residue was purified by trituration with pentane/ethyl ether/dichloromethane (5/5/1, 200 mL) to provide the desired product as a yellow solid (8.5 g, 36%). LCMS calculated for C₂₀H21N₂O₈S (M+H)⁺ m/z=449.1; found 449.1 (M+H)+; ¹H NMR (300 MHz, CDCl₃) δ 8.47 (d, J=1.8 Hz, 1H), 8.17 (dd, J=8.4, 1.8 Hz, 1H), 7.83 (d, J=8.1 Hz, 1H), 7.73-7.68 (m, 2H), 7.28-7.25 (m, 2H), 6.25 (s, 1H), 6.13 (d, J=8.7 Hz, 1H), 5.99 (d, J=8.7 Hz, 1H), 5.70 (s, 1H), 3.97 (s, 3H), 3.59 (s, 3H), 2.42 (s, 3H).

### Step 2: Methyl 4-(1-((N-allyl-4-methylphenyl) sulfonamido)-2-(methoxycarbonyl) allyl)-3-nitrobenzoate

To a mixture of methyl 4-(2-(methoxycarbonyl)-1-((4-methylphenyl) sulfonamido) allyl)-3-nitrobenzoate (7.5 g, 16.7 mmol) and potassium carbonate (23.11 g, 167.2 mmol) in N,N-dimethylformamide (200 mL) was added allyl bromide (20.23 g, 167.2 mmol) dropwise at room temperature. The resulting mixture was stirred at the same temperature for 16 h, and then filtered. The filtrate was diluted with ethyl acetate (1000 mL). The organic layer was successively washed with water (500 mL), brine (3×300 mL); and then dried with anhydrous sodium sulfate. After filtration, the filtrate was concentrated under reduced pressure. The residue was purified by silica gel column chromatography, eluted with 40% ethyl acetate in petroleum ether to provide the desired product as a yellow solid (7.4 g, 91%). LCMS calculated for C₂₃H25N₂O₈S (M+H)⁺ m/z=489.1; found 489.2 (M+H)⁺; ¹H NMR (400 MHz, CDCl₃) δ 8.56 (d, J=1.6 Hz, 1H), 8.28 (dd, J=8.0, 2.0 Hz, 1H), 7.92 (d, J=8.0 Hz, 1H), 7.67-763 (m, 2H), 7.26-7.24 (m, 2H), 6.79 (s, 1H), 6.45 (s, 1H), 5.59-5.49 (m, 1H), 5.47 (d, J=1.2 Hz, 1H), 5.04-4.96 (m, 2H), 4.25-4.19 (m, 1H), 4.00-3.94 (m, 4H), 3.53 (s, 3H), 2.42 (s, 3H).

### Step 3: Methyl 2-(4-(methoxycarbonyl)-2-nitrophenyl)-1-tosyl-2,5-dihydro-1H-pyrrole-3-carboxylate

To a mixture of methyl 4-(1-((N-allyl-4-methylphenyl) sulfonamido)-2-(methoxycarbonyl) allyl)-3-nitrobenzoate (6 g, 12.3 mmol) in dichloromethane (500 mL) was added tricyclohexylphosphine[1,3-bis(2,4,6-trimethylphenyl)-4,5-dihydroimidazol-2-ylidene][benzylidine]ruthenium (IV) dichloride (0.52 g, 0.6 mmol) under nitrogen atmosphere. The resulting mixture was stirred at room temperature for 16 h, followed by the addition of (methylsulfinyl) methane (2.4 g, 30.7 mmol). The mixture was stirred at the same temperature for additional 16 h, and then concentrated under reduced pressure. The residue was purified by silica gel column chromatography, eluted with 60% ethyl acetate in petroleum ether to provide the desired product as a white solid (6.1 g). LCMS calculated for C₂₁H₂₁N₂O₈S (M+H)⁺ m/z=461.1; found 460.9 (M+H)+; ¹H NMR (400 MHz, CDCl₃) δ 8.57 (d, J=2.0 Hz, 1H), 8.21 (dd, J=8.0, 1.6 Hz, 1H), 7.83-7.80 (m, 2H), 7.68 (d, J=8.0 Hz, 1H), 7.37-7.35 (m, 2H), 6.79-6.78 (m, 1H), 6.72-6.69 (m, 1H), 4.67-4.61 (m, 1H), 4.39-4.33 (m, 1H), 3.97 (s, 3H), 3.53 (s, 3H), 2.44 (s, 3H).

### Step 4: 4-(3-(Methoxycarbonyl)-1H-pyrrol-2-yl)-3-nitrobenzoic acid

The mixture of methyl 2-(4-(methoxycarbonyl)-2-nitrophenyl)-1-tosyl-2,5-dihydro-1H-pyrrole-3-carboxylate (3 g, 6.5 mmol) and potassium tert-butoxide (3.65 g, 32.5 mmol) in N,N-dimethylformamide (50 mL) was stirred for 2 h at room temperature, and then diluted with ethyl acetate (350 mL). The mixture was neutralized with aqueous potassium bisulfate (1%), washed with sodium bicarbonate (300 mL) and brine (200 mL). The aqueous layers were concentrated under vacuum. The residue was purified by reverse phase flash chromatography (column, C18 silica gel; mobile phase, acetonitrile in water (0.1% formic acid), 10% to 50% gradient over 30 min). The fractions were collected, combined and lyophilized to provide the desired product as a yellow solid (770 mg, 41%). LCMS calculated for C₁₃H₁₁N₂O₆ (M+H)⁺ m/z=291.1; found 290.9; ¹H NMR (400 MHz, DMSO-d₆) δ 13.70 (brs, 1H), 12.00 (s, 1H), 8.48 (d, J=1.6 Hz, 1H), 8.25 (dd, J=8.0, 1.6 Hz, 1H), 7.68 (d, J=8.0 Hz, 1H), 7.01 (t, J=2.8 Hz, 1H), 6.55 (t, J=2.8 Hz, 1H), 3.52 (s, 3H).

### Step 5: Methyl 2-(4-(methoxycarbonyl)-2-nitrophenyl)-1-methyl-1H-pyrrole-3-carboxylate

To a mixture of 4-(3-(methoxycarbonyl)-1H-pyrrol-2-yl)-3-nitrobenzoic acid (720 mg, 2.4 mmol) and cesium carbonate (3084 mg, 9.5 mmol) in N,N-dimethylformamide (30 mL) was added methyl iodide (1344 mg, 9.5 mmol) dropwise at room temperature. The resulting mixture was stirred at the same temperature for additional 2 h, and then diluted with ethyl acetate (150 mL). The organic layers were washed with brine (3×200 mL), and dried with anhydrous sodium sulfate. After filtration, the filtrate was concentrated under reduced pressure. The residue was purified by silica gel column chromatography, eluted with 50% ethyl acetate in petroleum ether to provide the desired product as a yellow solid (630 mg, 84%). LCMS calculated for C₁₅H₁₅N₂O₆ (M+H)⁺ m/z=319.1; found 319.1; ¹H NMR (400 MHz, CDCl₃) δ 8.74 (d, J=2.0 Hz, 1H), 8.33 (dd, J=8.0, 1.6 Hz, 1H), 7.50 (d, J=8.0 Hz, 1H), 6.74 (d, J=3.2 Hz, 1H), 6.66 (d, J=2.8 Hz, 1H), 4.01 (s, 3H), 3.59 (s, 3H), 3.45 (s, 3H).

### Step 6: Methyl 2-(2-amino-4-(methoxycarbonyl)phenyl)-1-methyl-1H-pyrrole-3-carboxylate

The mixture of methyl 2-(4-(methoxycarbonyl)-2-nitrophenyl)-1-methyl-1H-pyrrole-3-carboxylate (590 mg, 1.9 mmol), ammonium chloride (297 mg, 5.6 mmol) and iron (518 mg, 9.3 mmol) in ethanol (10 mL) and water (2 mL) was stirred at 80° C. for 2 h. Upon cooling to room temperature, the mixture was concentrated under vacuum. The residue was purified by silica gel column chromatography, eluted with 10% methanol in dichloromethane to provide the desired product as a yellow solid (519 mg, 97%). LCMS calculated for C₁₅H₁₇N₂O₄ (M+H)⁺ m/z=289.1; found 289.2; ¹H NMR (400 MHz, CDCl₃) δ 7.53-7.51 (m, 2H), 7.14 (d, J=7.6 Hz, 1H), 6.72 (d, J=2.8 Hz, 1H), 6.68 (d, J=2.8 Hz, 1H), 3.91 (s, 3H), 3.67 (s, 3H), 3.42 (s, 3H).

### Step 7: Methyl 1-methyl-4-oxo-4,5-dihydro-1H-pyrrolo[3,2-c]quinoline-7-carboxylate

The mixture of methyl 2-(2-amino-4-(methoxycarbonyl)phenyl)-1-methyl-1H-pyrrole-3-carboxylate (469 mg, 1.6 mmol) and acetic acid (1 mL) in 2-butanol (20 mL) was stirred at 100° C. for 16 h. Upon cooling to room temperature, the mixture was concentrated under vacuum. The residue was purified by silica gel column chromatography, eluted with 10% methanol in dichloromethane to provide the desired product as a yellow solid (324 mg, 78%). LCMS calculated for C₁₄H₁₃N₂O₃ (M+H)+m/z=257.1; found 257.0; ¹H NMR (400 MHz, DMSO-d₆) δ 11.46 (s, 1H), 8.27 (d, J=8.4 Hz, 1H), 8.06 (d, J=1.6 Hz, 1H), 7.75 (dd, J=8.4, 2.0 Hz, 1H), 7.32 (d, J=3.2 Hz, 1H), 6.66 (d, J=3.2 Hz, 1H), 4.19 (s, 3H), 3.89 (s, 3H).

### Step 8: 7-(Hydroxymethyl)-1-methyl-1,5-dihydro-4H-pyrrolo[3,2-c]quinolin-4-one

To the mixture of methyl 1-methyl-4-oxo-4,5-dihydro-1H-pyrrolo[3,2-c]quinoline-7-carboxylate (150 mg, 0.6 mmol) in anhydrous tetrahydrofuran (5 mL) was added lithium aluminum hydride (2.0 M in THF, 0.6 mL, 1.2 mmol) dropsies at 0° C. under nitrogen atmosphere. The resulting mixture was stirred at the same temperature for additional 1 h, and then quenched with water (one drop), 15% sodium hydroxide (one drop) and water (three drops) at 0° C., followed by the addition of anhydrous sodium sulfate (1 g). The mixture was stirred for 10 min at room temperature, filtered, and concentrated under vacuum. The residue was purified by reverse flash chromatography (column, C18 silica gel; mobile phase, methanol in water (10 mM ammonium bicarbonate), 0% to 100% gradient over 20 min). The fractions were collected, combined and lyophilized to provide the desired product as a white solid (70 mg, 53%). LCMS calculated for C₁₃H₁₃N₂O₂ (M+H)⁺ m/z=229.1; found 229.1.

### Step 9: N-methyl-5-(4-((1-methyl-4-oxo-4,5-dihydro-1H-pyrrolo[3,2-c]quinolin-7-yl)methyl)piperazin-1-yl)picolinamide (I-8)

The mixture of 7-(hydroxymethyl)-1-methyl-1,5-dihydro-4H-pyrrolo[3,2-c]quinolin-4-one (60 mg, 0.3 mmol) was combined with hydrogen bromide (33 wt. % solution in glacial acid, 2 mL) at room temperature; the reaction was then heated at 80° C. under nitrogen atmosphere for 2 h. Upon cooling to room temperature, the reaction was concentrated under reduced pressure. The residue was taken in acetonitrile (3 mL), followed by addition of N,N-diisopropylethylamine (272 mg, 2.1 mmol) and N-methyl-5-(piperazin-1-yl)pyridine-2-carboxamide dihydrochloride (116 mg, 0.4 mmol). Then the resulted mixture was heated at 70° C. for additional 2 h. The mixture was allowed to cool to room temperature and concentrated under reduced pressure. The residue was purified by prep-HPLC (column: XBridge Shield RP18 OBD column, 19*150 mm, 5 um; mobile phase A: water (10 mM ammonium bicarbonate), mobile phase B: acetonitrile; flow rate: 25 mL/min; gradient: 19% B to 43% B over 7 min); eluted fractions were collected and lyophilized to provide the desired product as a white solid (14 mg). LCMS calculated for C₂₄H₂₇N₆O₂ (M+H)⁺ m/z=431.2; found 431.0. ¹H NMR (400 MHz, DMSO-d₆) δ 11.19 (s, 1H), 8.41-8.37 (m, 1H), 8.27 (d, J=2.8 Hz, 1H), 8.12 (d, J=8.4 Hz, 1H), 7.83 (d, J=8.8 Hz, 1H), 7.41-7.37 (m, 2H), 7.20-7.14 (m, 2H), 6.58 (d, J=3.2 Hz, 1H), 4.14 (s, 3H), 3.60 (s, 2H), 3.36-3.33 (m, 4H), 2.78 (d, J=5.2 Hz, 3H), 2.57-2.54 (m, 4H).

### Example 9: 5-(4-((3-Ethyl-2,4-dioxo-1,2,3,4-tetrahydropyrido[3,2-d]pyrimidin-7-yl)methyl)piperazin-1-yl)-N-methylpicolinamide (I-9)

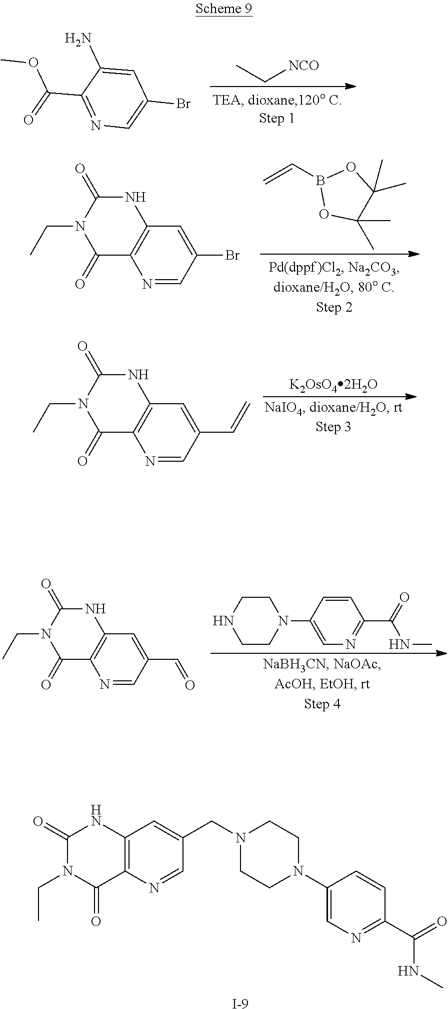

### Step 1: 7-Bromo-3-ethylpyrido[3,2-d]pyrimidine-2,4(1H,3H)-dione

To a mixture of methyl 3-amino-5-bromopicolinate (1 g, 4.3 mmol) in 1,4-dioxane (5 mL) were added isocyanatoethane (0.92 g, 13 mmol) and triethylamine (3.5 g, 34.6 mmol) at room temperature under nitrogen atmosphere. The resulting mixture was stirred at 120° C. for 16 h. Upon cooling to room temperature, the crude product precipitated out; it was collected by filtration followed by trituration with ethyl acetate (10 mL) and dried at room temperature to give the desired product as an off-white solid (1 g, 86%). LCMS calculated for C₉H₉BrN₃O₂ (M+H)⁺ m/z=270.0; found 269.9; ¹H NMR (300 MHz, DMSO-d₆) δ 8.56 (d, J=1.8 Hz, 1H), 7.76 (d, J=1.8 Hz, 1H), 3.92 (q, J=7.2 Hz, 2H), 1.15 (t, J=6.9 Hz, 3H).

### Step 2:3-Ethyl-7-vinylpyrido[3,2-d]pyrimidine-2,4(1H,3H)-dione

The mixture of 7-bromo-3-ethylpyrido[3,2-d]pyrimidine-2,4(1H,3H)-dione (300 mg, 1.1 mmol), 2-ethenyl-4,4,5,5-tetramethyl-1,3,2-dioxaborolane (342 mg, 2.2 mmol), sodium carbonate (235 mg, 2.2 mmol) and [1,1′-Bis(diphenylphosphino)ferrocene]palladium(II) chloride (81 mg, 0.1 mmol) in 1,4-dioxane (5 mL) and water (1 mL) was heated at 80° C. for 16 h under nitrogen atmosphere. Upon cooling to room temperature, the mixture was filtered. The solid was washed with water (3×50 mL) and methanol (2×10 mL); and then dried under vacuum to give the crude desired product as an off-white solid (200 mg). LCMS calculated for C₁₁H₁₂N₃O₂ (M+H)⁺ m/z=218.1; found 218.0.

### Step 3:3-Ethyl-2,4-dioxo-1,2,3,4-tetrahydropyrido[3,2-d]pyrimidine-7-carbaldehyde

To a mixture of 3-ethyl-7-vinylpyrido[3,2-d]pyrimidine-2,4(1H,3H)-dione (350 mg, 1.6 mmol) in 1,4-dioxane (6 mL) and water (2 mL) were added potassium osmate (VI) dihydrate (119 mg, 0.3 mmol) and sodium periodate (1.38 g, 6.4 mmol). The resulting mixture was stirred at room temperature for 2 h, and then filtered. The solid was washed with water (3×30 mL) and methanol (2×30 mL); and then dried under vacuum to give crude the desired product as an off-white solid (200 mg). LCMS calculated for C₁₀H₁₀N₃O₃ (M+H)⁺ m/z=220.1; found 220.1.

### Step 4: 5-(4-((3-Ethyl-2,4-dioxo-1,2,3,4-tetrahydropyrido[3,2-d]pyrimidin-7-yl)methyl)piperazin-1-yl)-N-methylpicolinamide (I-9)

The mixture of 3-ethyl-2,4-dioxo-1,2,3,4-tetrahydropyrido[3,2-d]pyrimidine-7-carbaldehyde (30 mg, 0.1 mmol), N-methyl-5-(piperazin-1-yl)picolinamide dihydrochloride (40 mg, 0.1 mmol) and sodium acetate (22.45 mg, 0.274 mmol) in ethanol (5 mL) was stirred for 20 min at room temperature, followed by the addition of acetic acid (16 mg, 0.3 mmol) and sodium cyanoborohydride (17 mg, 0.3 mmol).

The resulting mixture was stirred for 4 h, and then concentrated under reduced pressure and the residue was purified by prep-HPLC (column: YMC-Actus Triart C18, 30*150 mm, 5 um; mobile phase A: water (0.05% trifluoroacetic acid), mobile phase B: acetonitrile; flow rate: 60 mL/min; gradient: 5% B to 22% B over 5 min); eluted fractions were collected and lyophilized to provide the TFA salt of the desired product as a white solid (5 mg). LCMS calculated for C₂₁H₂₆N₇O₃ (M+H)⁺ m/z=424.2; found 424.0; ¹H NMR (300 MHz, CD₃OD) δ 8.63 (s, 1H), 8.38 (d, J=2.1 Hz, 1H), 7.99 (d, J=8.4 Hz, 1H), 7.86 (d, J=1.8 Hz, 1H), 7.51 (dd, J=9.0, 2.7 Hz, 1H), 4.59 (s, 2H), 4.13 (q, J=7.2 Hz, 2H), 3.71-3.66 (m, 4H), 3.54-3.49 (m, 4H), 2.96 (s, 3H), 1.29 (t, J=7.2 Hz, 3H).

### Example 10: N-methyl-5-(4-((3-methyl-4-oxo-4,5-dihydro-3H-pyrazolo[3,4-c]quinolin-7-yl)methyl)piperazin-1-yl)picolinamide (I-10)

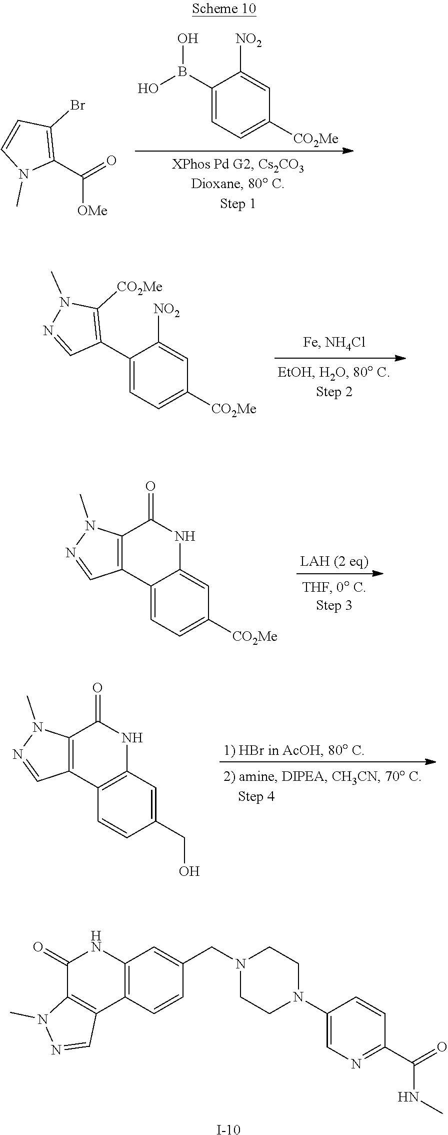

### Step 1: Methyl 4-(4-(methoxycarbonyl)-2-nitrophenyl)-1-methyl-1H-pyrazole-5-carboxylate

The mixture of methyl 4-bromo-1-methyl-1H-pyrazole-5-carboxylate (1 g, 4.6 mmol), (4-(methoxycarbonyl)-2-nitrophenyl) boronic acid (1.23 g, 5.5 mmol), cesium carbonate (2.97 g, 9.1 mmol), and chloro(2-dicyclohexylphosphino-2′,4′,6′-tri-i-propyl-1,1′-biphenyl)(2′-amino-1,1′-biphenyl-2-yl)palladium(II) (0.36 g, 0.5 mmol) in 1,4-dioxane (20 mL) was heated at 80° C. for 2 h under nitrogen atmosphere. Upon cooling to room temperature, the mixture was concentrated under vacuum, and the residue was purified by silica gel column chromatography, eluted with 40% ethyl acetate in petroleum to provide the desired product as an off-white solid (1.1 g, 76%). LCMS calculated for C₁₄H₁₄N₃O₆ (M+H)⁺ m/z=320.1; found 319.9; ¹H NMR (400 MHz, CDCl₃) δ 8.65 (d, J=1.6 Hz, 1H), 8.26 (dd, J=8.0, 1.6 Hz, 1H), 7.54 (s, 1H), 7.46 (d, J=7.6 Hz, 1H), 4.24 (s, 3H), 4.00 (s, 3H), 3.65 (s, 3H).

### Step 2: Methyl 3-methyl-4-oxo-4,5-dihydro-3H-pyrazolo[3,4-c]quinoline-7-carboxylate

The mixture of methyl 4-(4-(methoxycarbonyl)-2-nitrophenyl)-1-methyl-1H-pyrazole-5-carboxylate (1 g, 3.1 mmol), ammonium chloride (503 mg, 9.4 mmol) and iron (875 mg, 15.7 mmol) in ethanol (20 mL) and water (4 mL) was stirred at 80° C. for 2 h. Upon cooling to room temperature, the mixture was concentrated under vacuum and the residue was purified by silica gel column chromatography, eluted with 10% methanol in dichloromethane to give the desired product as a white solid (500 mg, 62%). LCMS calculated for C₁₃H₁₂N₃O₃ (M+H)⁺ m/z=258.1; found 258.0.

### Step 3: 7-(Hydroxymethyl)-3-methyl-3,5-dihydro-4H-pyrazolo[3,4-c]quinolin-4-one

To a mixture of methyl 3-methyl-4-oxo-4,5-dihydro-3H-pyrazolo[3,4-c]quinoline-7-carboxylate (200 mg, 0.8 mmol) in anhydrous tetrahydrofuran (8 mL) was added lithium aluminum hydride (2 M in tetrahydrofuran, 0.58 mL, 1.2 mmol) dropwise at 0° C. under nitrogen atmosphere. The resulting mixture was stirred at the same temperature for additional 1 h, and then quenched by the addition of water (one drop), 15% sodium hydroxide (one drop) and water (three drops) at 0° C., followed by the addition of anhydrous sodium sulfate (1 g). The mixture was stirred for 10 min at room temperature, filtered, and concentrated under vacuum. The residue was purified by reverse flash chromatography (column, C18 silica gel; mobile phase, methanol in water (10 mM ammonium bicarbonate), 10% to 50% gradient over 10 min). The fractions were collected, combined and lyophilized to provide the desired product as a white solid (65 mg, 36%). LCMS calculated for C₁₂H₁₂N₃O₂ (M+H)⁺ m/z=230.1; found 230.0.

### Step 4: N-methyl-5-(4-((3-methyl-4-oxo-4,5-dihydro-3H-pyrazolo[3,4-c]quinolin-7-yl)methyl)piperazin-1-yl)picolinamide (I-10)

The mixture of 7-(hydroxymethyl)-3-methyl-3,5-dihydro-4H-pyrazolo[3,4-c]quinolin-4-one (30 mg, 0.1 mmol), was combined with hydrogen bromide (33 wt. % solution in glacial acid, 2 mL) at room temperature; the reaction was then heated at 80° C. under nitrogen atmosphere for 1 h. Upon cooling to room temperature, the reaction was concentrated under reduced pressure. The residue was taken in acetonitrile (3 mL), followed by the addition of N,N-diisopropylethylamine (169 mg, 1.3 mmol) and N-methyl-5-(piperazin-1-yl)picolinamide dihydrochloride (42 mg, 0.1 mmol). Then the resulted mixture was heated at 70° C. for additional 2 h. The mixture was allowed to cool to room temperature and concentrated under reduced pressure. The residue was purified by prep-HPLC (Column: Kinetex EVO C18 Column, 21.2*150, 5 um; mobile phase A: water (0.05% trifluoroacetic acid), mobile phase B: acetonitrile; flow rate: 25 mL/min; gradient: 5% B to 25% B over 7 min); eluted fractions were collected and lyophilized to provide the TFA salt of the desired product as a white solid (38.7 mg). LCMS calculated for C₂₃H₂₆N₇O₂ (M+H)⁺ m/z=432.2; found 432.0; ¹H NMR (300 MHz, DMSO-d₆) δ 12.01 (s, 1H), 10.09 (s, 1H), 8.46 (s, 1H), 8.45-8.43 (m, 1H), 8.34 (d, J=3.0 Hz, 1H), 8.14 (d, J=8.1 Hz, 1H), 7.89 (d, J=8.7 Hz, 1H), 7.50-7.46 (m, 2H), 7.40 (dd, J=8.1, 1.5 Hz, 1H), 4.48 (s, 2H), 4.33 (s, 3H), 4.14-4.03 (m, 2H), 3.49-3.11 (m, 6H), 3.80 (d, J=4.8 Hz, 3H).

### Example 11: 5-(4-((3-Ethyl-2-oxo-1,2,3,4-tetrahydropyrido[3,2-d]pyrimidin-7-yl)methyl)piperazin-1-yl)-N-methylpicolinamide (I-11)

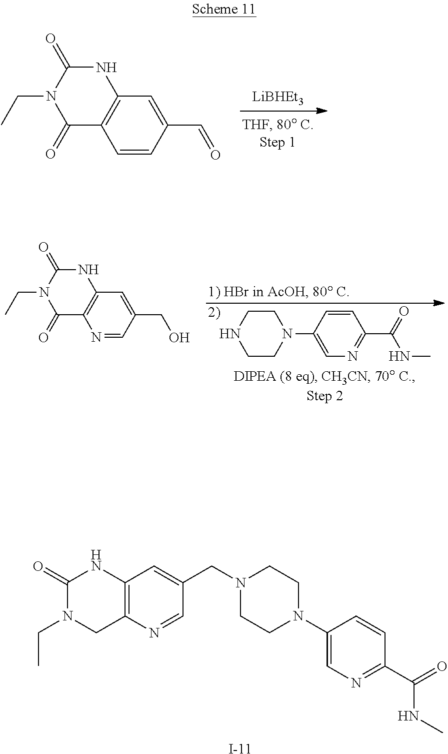

### Step 1:3-ethyl-7-(hydroxymethyl)-3,4-dihydropyrido[3,2-d]pyrimidin-2(1H)-one

To the mixture of 3-ethyl-2,4-dioxo-1,2,3,4-tetrahydropyrido[3,2-d]pyrimidine-7-carbaldehyde (200 mg, 0.9 mmol) in anhydrous tetrahydrofuran (10 mL) was added lithium triethylborohydride (1 M in tetrahydrofuran, 4.6 mL, 4.6 mmol) at 0° C. under nitrogen atmosphere. The resulting mixture was stirred for 1 h at 80° C. Upon cooling to 0° C., the mixture was quenched with saturated aqueous ammonium chloride solution (20 mL); the mixture was extracted with dichloromethane (3×100 mL). The combined organics were dried with anhydrous sodium sulfate, filtered, and concentrated under reduced pressure. The residue was purified by reverse flash chromatography (column, C18 silica gel; mobile phase, acetonitrile in water, 5% to 30% gradient over 15 min). The fractions were collected, combined and lyophilized to provide the desired product as a white solid (20 mg, 11%). LCMS calculated for C₁₀H₁₄N₃O₂ (M+H)⁺ m/z=208.1; found 208.1.

### Step 2: 5-(4-((3-ethyl-2-oxo-1,2,3,4-tetrahydropyrido[3,2-d]pyrimidin-7-yl)methyl)piperazin-1-yl)-N-methylpicolinamide (I-11)

The mixture of 3-ethyl-7-(hydroxymethyl)-3,4-dihydropyrido[3,2-d]pyrimidin-2(1H)-one (26 mg, 0.1 mmol) was combined with hydrogen bromide (33 wt. % solution in glacial acid, 1 mL) at room temperature; the reaction was then heated at 80° C. under nitrogen atmosphere for 2 h. Upon cooling to room temperature, the reaction was concentrated under reduced pressure. The residue was taken in acetonitrile (3 mL), followed by addition of N-methyl-5-(piperazin-1-yl)picolinamide dihydrochloride (37 mg, 0.1 mmol) and N-ethyl-N-isopropylpropan-2-amine (130 mg, 1 mmol). Then the resulted mixture was heated at 70° C. for additional 2 h. The mixture was allowed to cool to room temperature and concentrated under reduced pressure. The residue was purified by prep-HPLC (column: XBridge Prep OBD C18 Column, 30*150 mm, 5 um; mobile phase A: water (10 mmol/L ammonium bicarbonate), mobile phase B: acetonitrile; flow rate: 60 mL/min; gradient: 6% B to 32% B over 8.5 min). The fractions were collected, combined and lyophilized. The residue was purified by prep-HPLC again (column: YMC-Actus Triart C18, 30*150 mm, 5 um; mobile phase A: water (0.5% trifluoroacetate), mobile phase B: acetonitrile; flow rate: 60 mL/min; gradient: 5% B to 20% B over 5 min); eluted fractions were collected and lyophilized to give the TFA salt of the desired product (5.6 mg) as a white solid. LCMS calculated for C₂₁H₂₈N₇O₂ (M+H)⁺ m/z=410.2; found 410.1; ¹H NMR (400 MHz, CD₃OD) δ 8.36 (d, J=2.8 Hz, 1H), 8.23 (d, J=2.0 Hz, 1H), 7.97 (d, J=8.8 Hz, 1H), 7.49 (dd, J=8.8, 3.2 Hz, 1H), 7.31 (d, J=2.0 Hz, 1H), 4.64 (s, 2H), 4.43 (s, 2H), 3.75-3.59 (m, 4H), 3.52-3.47 (m, 6H), 2.93 (s, 3H), 1.22 (t, J=7.2 Hz, 3H).

### Example 12: 5-(4-((3-(2,2-Difluoroethyl)-2,4-dioxo-1,2,3,4-tetrahydroquinazolin-7-yl)methyl)piperazin-1-yl)-N-methylpicolinamide (I-12)

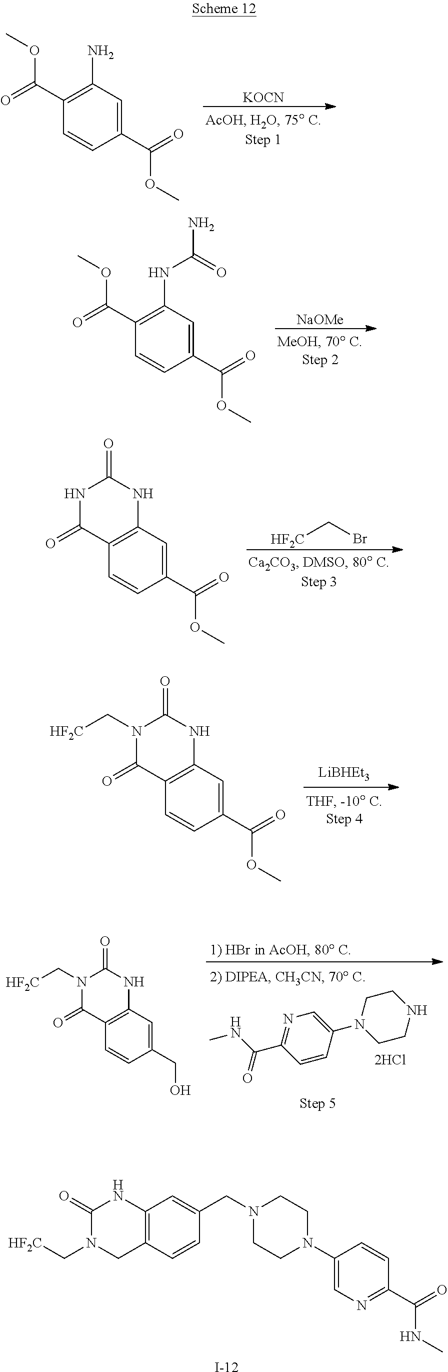

### Step 1: Dimethyl 2-ureidoterephthalate

The mixture of dimethyl 2-aminoterephthalate (3 g, 14.3 mmol) and potassium cyanate (4.07 g, 50.2 mmol) in glacial acetic acid (20 mL) was stirred at 75° C. for 16 h under nitrogen atmosphere. Upon cooling to room temperature, the crude product precipitated out; it was collected by filtration followed by washing with water (2×100 mL) and dried at room temperature to give the desired product as a white solid (2.5 g, 69%). LCMS calculated for C₁₁H₁₃N₂O₅ (M+H)⁺ m/z=253.1; found 253.0.

### Step 2: Methyl 2,4-dioxo-1,2,3,4-tetrahydroquinazoline-7-carboxylate

The mixture of dimethyl 2-ureidoterephthalate (1.5 g, 5.9 mmol) and sodium methoxide (0.64 g, 11.9 mmol) in methanol (30 mL) was stirred at 75° C. for 16 h under nitrogen atmosphere. Upon cooling to 0° C., the mixture was acidified to pH=2 with hydrogen chloride (1 M). The precipitated solids were collected by filtration and washed with water (2×20 mL), methanol (2×20 mL), diethyl ether (2×20 mL); and then dried under vacuum to give the desired product as a white solid (850 mg, 65%). LCMS calculated for C₁₀H₉N₂O₄ (M−H)⁺ m/z=221.1; found 221.1; ¹H NMR (400 MHz, DMSO-d₆) δ 11.48 (s, 1H), 11.32 (s, 1H), 8.00 (d, J=8.4 Hz, 1H), 7.75 (d, J=1.6 Hz, 1H), 7.67 (dd, J=8.4, 1.6 Hz, 1H), 3.90 (s, 3H).

### Step 3: Methyl 3-(2,2-difluoroethyl)-2,4-dioxo-1,2,3,4-tetrahydroquinazoline-7-carboxylate

The mixture of methyl 2,4-dioxo-1,3-dihydroquinazoline-7-carboxylate (150 mg, 0.7 mmol), 2-bromo-1,1-difluoroethane (147 mg, 1.0 mmol) and cesium carbonate (333 mg, 1.0 mmol) in dimethyl sulfoxide (5 mL) was stirred at 80° C. for 16 h. Upon cooling to room temperature, the mixture was filtered and the filtrate was purified by reverse flash chromatography (column, C18 silica gel; mobile phase, acetonitrile in water (5 mM ammonia), gradient 10% to 50% over 10 min). The fractions were collected, combined and lyophilized to provide the desired product as a white solid (100 mg, 52%). LCMS calculated for C₁₂H₁₁F₂N₂O₄ (M−H)⁺ m/z=285.1; found 285.1.

### Step 4:3-(2,2-difluoroethyl)-7-(hydroxymethyl) quinazoline-2,4(1H,3H)-dione

To the mixture of methyl 3-(2,2-difluoroethyl)-2,4-dioxo-1,2,3,4-tetrahydroquinazoline-7-carboxylate (100 mg, 0.4 mmol) in anhydrous tetrahydrofuran (5 mL) was added lithium triethylborohydride (1 M in tetrahydrofuran, 1.1 mL, 1.1 mmol) dropwise at −10° C. under nitrogen atmosphere. The resulting mixture was stirred at the same temperature for additional 30 min; and then quenched with saturated aqueous ammonium chloride solution (10 mL). The mixture was extracted with dichloromethane (2×20 mL). The combined organics were dried with anhydrous sodium sulfate, filtered, and concentrated under reduced pressure. The residue was purified by silica gel column chromatography, eluted with 10% methanol in dichloromethane to provide the desired product as a white solid (47 mg, 52%). LCMS calculated for C₁₁H₁₁F₂N₂O₃ (M−H)⁺ m/z=257.1; found 257.1; ¹H NMR (300 MHz, DMSO-d₆) δ 11.62 (s, 1H), 7.90 (d, J=8.1 Hz, 1H), 7.21 (d, J=1.5 Hz, 1H), 7.15 (dd, J=8.1, 1.5 Hz, 1H), 6.47-6.06 (m, 1H), 5.49 (t, J=5.7 Hz, 1H), 4.59 (d, J=5.7 Hz, 2H), 4.38-4.27 (m, 2H).

### Step 5: 5-(4-((3-(2,2-difluoroethyl)-2,4-dioxo-1,2,3,4-tetrahydroquinazolin-7-yl)methyl)piperazin-1-yl)-N-methylpicolinamide (I-12)

The mixture of 3-(2,2-difluoroethyl)-7-(hydroxymethyl) quinazoline-2,4(1H,3H)-dione (20 mg, 0.1 mmol) was combined with hydrogen bromide (33 wt. % solution in glacial acid, 1 mL) at room temperature; the reaction was then heated at 80° C. under nitrogen atmosphere for 2 h. Upon cooling to room temperature, the reaction was concentrated under reduced pressure. The residue was taken in acetonitrile (1 mL), followed by the addition of N-ethyl-N-isopropylpropan-2-amine (101 mg, 0.8 mmol) and N-methyl-5-(piperazin-1-yl)pyridine-2-carboxamide dihydrochloride (24 mg, 0.1 mmol). Then the resulted mixture was heated at 70° C. for additional 2 h. The mixture was allowed to cool to room temperature and concentrated under reduced pressure. The residue was purified by prep-HPLC (column: Xselect CSH C18 OBD column 30*150 mm 5 um; mobile phase A: water (0.5% trifluoroacetate), mobile phase B: acetonitrile; flow rate: 60 mL/min; gradient: 5% B to 35% B over 9 min); eluted fractions were collected and lyophilized to give the TFA salt of the desired product as a white solid (9 mg). LCMS calculated for C₂₂H₂₅F₂N₆O₃ (M+H)⁺ m/z=459.2; found 459.0; ¹H NMR (400 MHz, DMSO-d₆) δ 11.91 (s, 1H), 10.19 (s, 2H), 8.47-8.43 (m, 1H), 8.34 (d, J=2.8 Hz, 1H), 8.07 (d, J=8.0 Hz, 1H), 7.89 (d, J=8.8 Hz, 1H), 7.48 (dd, J=8.8, 2.8 Hz, 1H), 7.37 (d, J=8.0 Hz, 1H), 7.32 (s, 1H), 6.42-6.12 (m, 1H), 4.49 (s, 2H), 4.39-4.31 (m, 2H), 4.14-4.00 (m, 2H), 3.46-3.11 (m, 6H), 2.79 (d, J=4.8 Hz, 3H); ¹⁹F NMR (377 MHz, DMSO-d₆) δ −74.48, 120.97.

### Example 13: 5-(4-((6-Ethyl-5-oxo-4,5-dihydrothieno[3,2-b]pyridin-2-yl)methyl)piperazin-1-yl)-N-methylpicolinamide (I-13)

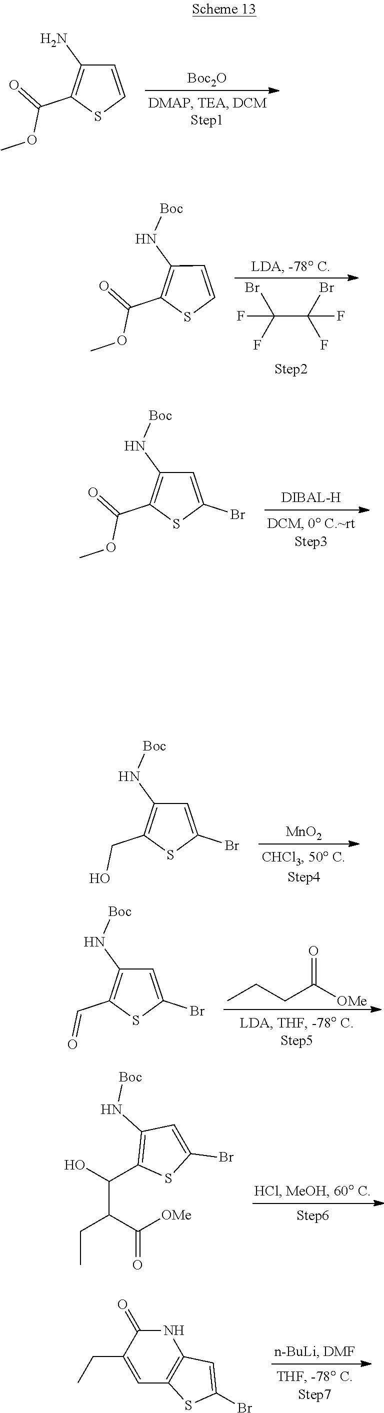

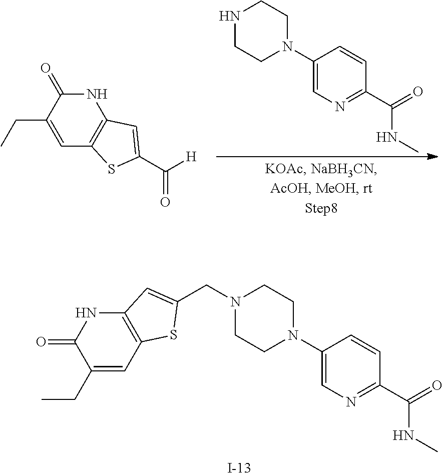

### Step 1: Methyl 3-((tert-butoxycarbonyl)amino)thiophene-2-carboxylate

To a mixture of methyl 3-aminothiophene-2-carboxylate (19 g, 120.87 mmol) and triethylamine (14.68 g, 145 mmol) in dichloromethane (100 mL) were added di-tert-butyl dicarbonate (28.76 g, 133 mmol) and 4-dimethylaminopyridine (0.74 g, 6 mmol); the reaction was stirred at room temperature for 16 h, and then diluted with water (100 mL) and extracted with dichloromethane (3×50 mL). The combined organic layers were washed with brine (3×30 mL), dried with anhydrous sodium sulfate, filtered, and concentrated under reduced pressure. The residue was purified by silica gel column chromatography, eluted with 50% ethyl acetate in petroleum ether to provide the desired product as a white solid (11.2 g, 36%). ¹H NMR (300 MHz, CDCl₃) δ 9.37 (s, 1H), 7.97 (d, J=5.4 Hz, 1H), 7.45 (d, J=5.4 Hz, 1H), 3.90 (s, 3H), 1.54 (s, 9H).

### Step 2: Methyl 5-bromo-3-((tert-butoxycarbonyl)amino)thiophene-2-carboxylate

To a mixture of diisopropylamine (2.83 g, 27.98 mmol) in anhydrous tetrahydrofuran (10 mL) was added n-butyllithium (2.5 M in hexane, 10.88 mL, 27.21 mmol) dropwise at −10° C. under nitrogen atmosphere. The mixture was stirred at −10° C. for 1 h. The fresh made lithium diisopropylamide solution solution was used below. To a mixture of methyl 3-((tert-butoxycarbonyl)amino)thiophene-2-carboxylate (2 g, 7.77 mmol) in anhydrous tetrahydrofuran (10 mL) was added lithium diisopropylamide solution (prepared above) dropwise at −78° C. under nitrogen atmosphere. The mixture was stirred at the same temperature for 1.5 h; followed by the addition of dibromotetrafluoroethane (12.12 g, 46.64 mmol) dropwise at −78° C. The mixture was stirred at −78° C. for additional 2 h; then quenched with saturated aqueous ammonium chloride (100 mL) at 0° C., extracted with ethyl acetate (3×100 mL). The combined organic layers were washed with brine (3×50 mL), dried with anhydrous sodium sulfate. After filtration, the filtrate was concentrated under reduced pressure. The residue was purified by reverse flash chromatography (column, C₁₈ silica gel; mobile phase, acetonitrile in water, 10% to 50% gradient over 30 min). Collected fractions and concentrated under vacuum to provide the desired product as a white solid (1.1 g, 42%). ¹H NMR (300 MHz, CDCl₃) δ 9.34 (s, 1H), 7.98 (s, 1H), 3.87 (s, 3H), 1.53 (s, 9H).

### Step 3: tert-Butyl(5-bromo-2-(hydroxymethyl)thiophen-3-yl) carbamate

To a mixture of methyl 5-bromo-3-((tert-butoxycarbonyl)amino)thiophene-2-carboxylate (1.1 g, 3.27 mmol) in dichloromethane (15 mL) was added diisobutyl aluminium hydride (1.5 M in toluene, 10.91 mL, 16.37 mmol) dropwise at 0° C. under nitrogen atmosphere; the reaction was stirred at room temperature for 2 h; then quenched with water (1 mL), 15% sodium hydroxide (0.5 mL) and water (1 mL) at 0° C. The mixture was stirred at room temperature and filtered. The filtrate was concentrated under reduced pressure to give the desired product as a yellow solid (440 mg, 44%). ¹H NMR (300 MHz, CDCl₃) δ 7.22 (s, 1H), 6.69 (s, 1H), 4.61 (s, 2H), 1.53 (s, 9H).

### Step 4: tert-Butyl(5-bromo-2-formylthiophen-3-yl) carbamate

The mixture of tert-butyl(5-bromo-2-(hydroxymethyl)thiophen-3-yl) carbamate (440 mg, 1.43 mmol) and manganese dioxide (1.24 g, 14.26 mmol) in chloroform (8 mL) was stirred at 50° C. for 16 h. Upon cooling to room temperature, the mixture was filtered and the filtrate was concentrated under vacuum. The residue was purified by silica gel column chromatography, eluted with 30% ethyl acetate in petroleum ether to provide the desired product as a yellow solid (318 mg, 73%). 1H NMR (400 MHz, CDCl₃) δ 9.82 (s, 1H), 9.51 (s, 1H), 8.02 (s, 1H), 1.52 (s, 9H).

### Step 5: Methyl 2-((5-bromo-3-((tert-butoxycarbonyl)amino)thiophen-2-yl)(hydroxy)methyl)butanoate

To a solution of diisopropylamine (256 mg, 2.53 mmol) in anhydrous tetrahydrofuran (2 mL) was added n-butyllithium (2.5 M in hexane, 0.98 mL, 2.451 mmol) dropwise at −78° C. under nitrogen atmosphere. The mixture was stirred at 0° C. for 10 min, followed by the addition of methyl butyrate (250 mg, 2.45 mmol) dropwise at −78° C.; and the mixture was stirred at the same temperature for additional 1 h. Then tert-butyl(5-bromo-2-formylthiophen-3-yl) carbamate (250 mg, 0.82 mmol) in anhydrous tetrahydrofuran (2.5 mL) was added dropwise at −78° C. The resulting mixture was stirred at the same temperature for additional 30 min; and then quenched with saturated aqueous ammonium chloride at 0° C., extracted with ethyl acetate (3×10 mL). The combined organic layers were washed with water (2×10 mL) and brine (2×10 mL), dried with anhydrous sodium sulfate. After filtration, the filtrate was concentrated under reduced pressure to provide the desired product as a yellow solid (318 mg, crude). LCMS calculated for C₁₅H₂₂BrNO₅SNa (M+Na)⁺ m/z=430.0; found 429.9.

### Step 6:2-Bromo-6-ethylthieno[3,2-b]pyridin-5 (4H)-one

The mixture of methyl 2-((5-bromo-3-((tert-butoxycarbonyl)amino)thiophen-2-yl)(hydroxy)methyl)butanoate (268 mg, 0.66 mmol) in dioxane (10 mL) was treated with hydrogen chloride (4 M in dioxane, 0.2 mL, 0.8 mmol); the reaction was stirred at 60° C. for 2 h. Upon cooling to room temperature, the mixture was neutralized with saturated aqueous sodium bicarbonate, and extracted with ethyl acetate (3×30 mL). The combined organic layers were washed with brine (2×20 mL), dried with anhydrous sodium sulfate. After filtration, the filtrate was concentrated under reduced pressure. The residue was purified by silica gel column chromatography, eluted with 30% ethyl acetate in petroleum ether to provide the desired product as a yellow solid (102 mg, 60%). LCMS calculated for C₉H₉BrNOS (M+H)⁺ m/z=258.0; found 257.9.

### Step 7: 6-Ethyl-5-oxo-4,5-dihydrothieno[3,2-b]pyridine-2-carbaldehyde

To the mixture of 2-bromo-6-ethylthieno[3,2-b]pyridin-5 (4H)-one (48 mg, 0.19 mmol) was added n-butyllithium (2.5 M in hexane, 0.22 mL, 0.56 mmol) at −78° C. under nitrogen atmosphere; the reaction was stirred for 1 h, followed by the addition of N,N-dimethylformamide (2 mL). The reaction was stirred at the same temperature for 1 h, and then concentrated under reduced pressure. The residue was purified by reverse flash chromatography (column, C18 silica gel; mobile phase, acetonitrile in water (0.1% trifluoroacetic acid), 5% to 95% gradient over 40 min). The fractions were collected, combined and lyophilized to provide the desired product as a yellow solid (16 mg, 55%). LCMS calculated for C10H10NO₂S (M+H)⁺ m/z=208.0; found 208.0.

### Step 8: 5-(4-((6-Ethyl-5-oxo-4,5-dihydrothieno[3,2-b]pyridin-2-yl)methyl)piperazin-1-yl)-N-methylpicolinamide (I-13)

The mixture of 6-ethyl-5-oxo-4,5-dihydrothieno[3,2-b]pyridine-2-carbaldehyde (16 mg, 0.077 mmol), potassium acetate (25 mg, 0.26 mmol) and N-methyl-5-(piperazin-1-yl)pyridine-2-carboxamide dihydrochloride (23 mg, 0.077 mmol, 1 eq) in ethanol (3 mL) was stirred at room temperature for 20 min, followed by the addition of acetic acid (0.1 mL, 1.75 mmol) and sodium cyanoborohydride (10 mg, 0.15 mmol). The mixture was stirred for 2 h; and then concentrated under reduced pressure. The residue was purified by reverse flash chromatography (column: XBridge Shield RP18 OBD Column, 19*150 mm, 5 um; mobile phase A: water (10 mmol/L ammonium bicarbonate), mobile phase B: methanol; flow rate: 25 mL/min; Gradient: 41% B to 70% B over 8 min); eluted fractions were collected and lyophilized to provide the desired product as a white solid (4.3 mg). LCMS calculated for C₂₁H₂₆N₅O₂S (M+H)⁺ m/z=412.2; found 412.0; ¹H NMR (400 MHz, DMSO-d₆) δ 12.01 (s, 1H), 8.41-8.37 (m, 1H), 8.26 (d, J=2.8 Hz, 1H), 7.82 (d, J=8.8 Hz, 1H), 7.75 (s, 1H), 7.39 (dd, J=8.8, 2.8 Hz, 1H), 6.88 (s, 1H), 3.80 (s, 2H), 3.35-3.32 (m, 4H), 2.78 (d, J=4.8 Hz, 3H), 2.61-2.59 (m, 4H), 2.45 (q, J=7.6 Hz, 2H), 1.12 (t, J=7.6 Hz, 3H).

### Example 14: 5-(4-((4-Ethyl-5-oxo-2,3,5,6-tetrahydropyrano[4,3,2-de]quinolin-8-yl)methyl)piperazin-1-yl)-N-methylpicolinamide (I-14)

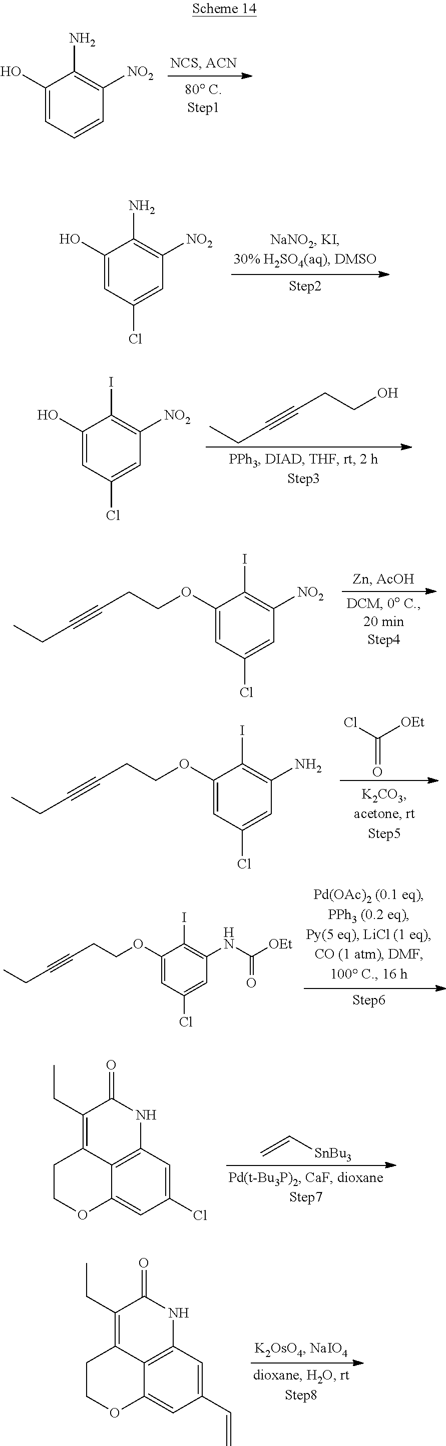

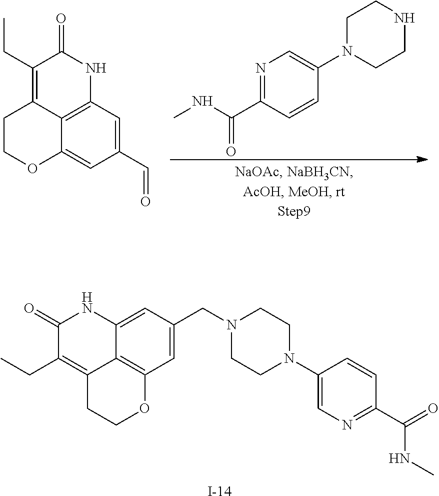

### Step 1:2-Amino-5-chloro-3-nitrophenol

The solution of 2-amino-3-nitrophenol (3 g, 19.4 mmol) and N-chlorosuccinimide (3.12 g, 23.3 mmol) in acetonitrile (100 mL) was refluxed for 3 h. Upon cooling to room temperature, the resulting mixture was concentrated under reduced pressure. The residue was diluted with ethyl acetate (3×100 mL), washed with water (3×100 mL), dried over anhydrous sodium sulfate. After filtration, the filtrate was concentrated under reduced pressure. The residue was purified by silica gel column chromatography, eluted with 30% ethyl acetate in petroleum ether to provide the desired product as a white solid (3.4 g, 92%). LCMS calculated for C₆H₆ClN₂O₃ (M+H)⁺ m/z=189.0; found 188.9; ¹H NMR (400 MHz, DMSO-d₆) δ 11.06 (s, 1H), 7.47 (d, J=2.4 Hz, 1H), 7.04 (s, 2H), 6.86 (d, J=2.4 Hz, 1H).

### Step 2: 5-Chloro-2-iodo-3-nitrophenol

The solution of 2-amino-5-chloro-3-nitrophenol (3 g, 15.9 mmol) in dimethyl sulfoxide (50 mL) and sulfuric acid (30% in water) (50 mL) heated at 50° C. for 1 h. Upon cooling to 0° C., to the above mixture was added sodium nitrite (1.6 g, 23.2 mmol) in water (5 mL) dropwise at 0° C. The resulting mixture was stirred at the same temperature for additional 1 h; followed by the addition of potassium iodide (6.4 g, 38.5 mmol) in water (10 mL) dropwise at 0° C. The resulting mixture was stirred at room temperature for 48 h; and then neutralized with sodium bisulfite, extracted with ethyl acetate (3×70 mL). The combined organic layers were dried with anhydrous sodium sulfate, filtered, and concentrated under reduced pressure. The residue was purified by reverse flash chromatography (column, C18 silica gel; mobile phase, acetonitrile in water (0.1% ammonium hydroxide), 10% to 50% gradient over 50 min). Collected fractions and concentrated under vacuum to provide the desired product as a brown solid (2 g, 42%). ¹H NMR (400 MHz, CDCl₃) δ 7.46 (d, J=2.4 Hz, 1H), 7.25 (d, J=2.4 Hz, 1H), 6.09 (s, 1H).

### Step 3: 5-Chloro-1-(hex-3-yn-1-yloxy)-2-iodo-3-nitrobenzene

To a solution of triphenylphosphine (1.27 g, 4.7 mmol) in tetrahydrofuran (20 mL) was added diisopropyl azodicarboxylate (0.97 g, 4.76 mmol) dropwise at 0° C. under nitrogen atmosphere; The mixture was stirred at the same temperature until the mixture became milky, followed by the addition of 5-chloro-2-iodo-3-nitrophenol (1 g, 3.2 mmol) and 3-hexyn-1-ol (0.38 g, 3.8 mmol) in tetrahydrofuran (5 mL). The mixture was allowed to stir at room temperature for 2 h; and then quenched with water (20 mL), extracted with ethyl acetate (3×20 mL). The combined organic layers were dried with anhydrous sodium sulfate, filtered, and concentrated under reduce pressure. The residue was purified by silica gel column chromatography, eluted with 20% ethyl acetate in petroleum ether to provide the desired product as a white solid (400 mg, 33%). LCMS calculated for C₁₂H₁₂ClINO₃ (M+H)⁺ m/z=380.0; found 379.8. ¹H NMR (300 MHz, CDCl₃) δ 7.34 (d, J=2.1 Hz, 1H), 6.99 (d, J=2.1 Hz, 1H), 4.18 (t, J=7.2 Hz, 2H), 2.81-2.73 (m, 2H), 2.25-2.15 (m, 2H), 1.15 (t, J=7.5 Hz, 3H).

### Step 4: 5-Chloro-3-(hex-3-yn-1-yloxy)-2-iodoaniline

The mixture of 5-chloro-1-(hex-3-yn-1-yloxy)-2-iodo-3-nitrobenzene (500 mg, 1.3 mmol) and zinc (861.2 mg, 13.0 mmol) in dichloromethane (20 mL) was added acetic acid (791.0 mg, 13.0 mmol) dropwise at 0° C. under nitrogen atmosphere. The resulting mixture was stirred at the same temperature for 20 min; and then filtered, the filter cake was washed with ethyl acetate (3×20 mL). The filtrate was neutralized with saturated aqueous sodium bicarbonate solution, extracted with ethyl acetate (3×30 mL). The combined organic layers were washed with brine (2×30 mL), dried with anhydrous sodium sulfate. After filtration, the filtrate was concentrated under reduced pressure. The filtrate was evaporated under reduced pressure to provide the desired product (460 mg, crude). LCMS calculated for C₁₂H₁₄ClINO (M+H)⁺ m/z=350.0; found 350.0.

### Step 5: Ethyl(5-chloro-3-(hex-3-yn-1-yloxy)-2-iodophenyl) carbamate

The mixture of 5-chloro-3-(hex-3-yn-1-yloxy)-2-iodoaniline (460 mg, 1.3 mmol) and potassium carbonate (909 mg, 6.6 mmol) in acetone (10 mL) was added ethyl chloroformate (5.0 g, 46.1 mmol) dropwise at room temperature. The resulting mixture was stirred for 3 days; and then diluted with water (20 mL), extracted with ethyl acetate (3×20 mL). The combined organic layers were washed with brine (2×20 mL), dried with anhydrous sodium sulfate. After filtration, the filtrate was concentrated under reduced pressure. The residue was purified by silica gel column chromatography, eluted with 10% ethyl acetate in petroleum ether to provide the desired product as a white solid (300 mg, 54%). LCMS calculated for C₁₅H₁₈ClINO₃ (M+H)⁺ m/z=422.0; found 421.8; ¹H NMR (400 MHz, CDCl₃) δ 7.88 (d, J=2.4 Hz, 1H), 7.21 (s, 1H), 6.56 (d, J=2.4 Hz, 1H), 4.25 (q, J=7.2 Hz, 2H), 4.09 (t, J=7.2 Hz, 2H), 2.72-2.66 (m, 2H), 2.21-2.13 (m, 2H), 1.34 (t, J=7.2 Hz, 3H), 1.13 (t, J=7.6 Hz, 3H).

### Step 6: 8-Chloro-4-ethyl-2,3-dihydropyrano[4,3,2-de]quinolin-5 (6H)-one

The mixture of ethyl(5-chloro-3-(hex-3-yn-1-yloxy)-2-iodophenyl) carbamate (310 mg, 0.7 mmol), triphenylphosphane (39 mg, 0.15 mmol)palladium acetate (16 mg, 0.07 mmol), lithium chloride (31 mg, 0.7 mmol) and pyridine (291 mg, 3.7 mmol) in N,N-dimethylformamide (10 mL) was bubbled with carbon monoxide for 2 min; and then heated at 100° C. for 16 h. Upon cooling to room temperature, the mixture was diluted with ethyl acetate, washed with brine, dried and filtered. The filtrate was evaporated under reduced pressure. The residue was treated with 10 mL of 1 M sodium hydroxide in water at room temperature for 1 h. The mixture was diluted with ethyl acetate, washed with brine, dried and filtered. The residue was purified by silica gel column chromatography, eluted with 60% ethyl acetate in petroleum ether to provide the desired product as a white solid (150 mg, 82%). LCMS calculated for C₁₃H₁₃ClNO₂ (M+H)⁺ m/z=250.1; found 250.1; ¹H NMR (400 MHz, DMSO-d₆) δ 11.69 (s, 1H), 6.85 (d, J=2.0 Hz, 1H), 6.68 (d, J=2.0 Hz, 1H), 4.31 (t, J=6.0 Hz, 2H), 3.01 (t, J=6.0 Hz, 2H), 2.54 (q, J=7.6 Hz, 2H), 1.01 (t, J=7.6 Hz, 3H).

### Step 7: 4-Ethyl-8-vinyl-2,3-dihydropyrano[4,3,2-de]quinolin-5 (6H)-one

The mixture of 8-chloro-4-ethyl-2,3-dihydropyrano[4,3,2-de]quinolin-5 (6H)-one (140 mg, 0.56 mmol), tributyl(ethenyl) stannane (267 mg, 0.8 mmol), cesium fluoride (187 mg, 1.2 mmol) and bis(tri-tert-butylphosphine)palladium(0) (29 mg, 0.06 mmol) in 1,4-dioxane (3 mL) was stirred for 16 h at 100° C. under nitrogen atmosphere. Upon cooling to room temperature, the mixture was filtered; the filter cake was washed with ethyl acetate (3×20 mL). The filtrate was concentrated under reduced pressure. The residue was purified by Prep-TLC, eluted with 50% ethyl acetate in petroleum ether to provide the desired product as a white solid (65 mg, 48%). LCMS calculated for C₁₅H₁₆NO₂ (M+H)⁺ m/z=242.1; found 242.1; ¹H NMR (400 MHz, CDCl₃) δ 10.69 (s, 1H), 6.87 (s, 2H), 6.70 (dd, J=17.6, 10.8 Hz, 1H), 5.86 (d, J=17.6 Hz, 1H), 5.40 (d, J=10.8 Hz, 1H), 4.39 (t, J=6.0 Hz, 2H), 3.09 (t, J=6.0 Hz, 2H), 2.70 (q, J=7.6 Hz, 2H), 1.16 (t, J=7.6 Hz, 3H).

### Step 8: 4-Ethyl-5-oxo-2,3,5,6-tetrahydropyrano[4,3,2-de]quinoline-8-carbaldehyde

To the mixture of 4-ethyl-8-vinyl-2,3-dihydropyrano[4,3,2-de]quinolin-5 (6H)-one (60 mg, 0.25 mmol) in 1,4-dioxane (6 mL) and water (2 mL) were added sodium periodate (213 mg, 1.0 mmol) and potassium osmate (VI) dihydrate (18 mg, 0.05 mmol). The resulting mixture was stirred at room temperature for 2 h; and then diluted with water (20 mL), extracted with ethyl acetate (3×20 mL). The combined organic layers were washed with brine (1×20 mL), dried with anhydrous sodium sulfate. After filtration, the filtrate was concentrated under reduced pressure. The residue was purified by Prep-TLC, eluted with 50% ethyl acetate in petroleum ether to provide the desired product as a light-yellow solid (40 mg, 66%). LCMS calculated for C₁₄H₁₄NO₃ (M+H)⁺ m/z=244.1; found 244.1; ¹H NMR (400 MHz, CDCl₃) δ 11.36 (s, 1H), 10.01 (s, 1H), 7.38 (d, J=1.2 Hz, 1H), 7.19 (d, J=1.2 Hz, 1H), 4.41 (t, J=6.0 Hz, 2H), 3.10 (t, J=6.0 Hz, 2H), 2.75 (q, J=7.6 Hz, 2H), 1.20 (t, J=7.6 Hz, 3H).

### Step 9: 5-(4-((4-Ethyl-5-oxo-2,3,5,6-tetrahydropyrano[4,3,2-de]quinolin-8-yl)methyl)piperazin-1-yl)-N-methylpicolinamide (I-14)

The mixture of 4-ethyl-5-oxo-2,3,5,6-tetrahydropyrano[4,3,2-de]quinoline-8-carbaldehyde (35 mg, 0.14 mmol), sodium acetate (47.2 mg, 0.58 mmol) and N-methyl-5-(piperazin-1-yl)picolinamide dihydrochloride (44 mg, 0.15 mmol) in methanol (2 mL) was stirred at room temperature for 30 min; followed by the addition of acetic acid (26 mg, 0.42 mmol) and sodium cyanoborohydride (18 mg, 0.28 mmol). The resulting mixture was stirred for additional 2 h; and then concentrated under reduced pressure. The residue was purified by reverse flash chromatography (column: Xselect CSH C18 OBD Column 30*150 mm 5 um; mobile phase A: water (0.05% trifluoroacetic acid), mobile phase B: acetonitrile; flow rate: 60 mL/min; gradient: 10% B to 35% B over 7 min); eluted fractions were collected and lyophilized to provide the TFA salt of the desired product as a white solid (9.9 mg). LCMS calculated for C₂₅H₃₀N₅O₃ (M+H)⁺ m/z=448.2; found 448.2. ¹H NMR (400 MHz, DMSO-d₆) δ 11.91 (s, 1H), 9.94 (s, 1H), 8.48-8.42 (m, 1H), 8.33 (d, J=2.8 Hz, 1H), 7.89 (d, J=8.8 Hz, 1H), 7.47 (dd, J=8.8, 2.8 Hz, 1H), 6.91 (d, J=1.2 Hz, 1H), 6.79 (d, J=1.2 Hz, 1H), 4.39 (s, 2H), 4.33 (t, J=5.6 Hz, 2H), 4.13-4.02 (m, 2H), 3.49-3.37 (m, 2H), 3.26-3.10 (m, 4H), 3.04 (t, J=6.0 Hz, 2H), 2.79 (d, J=4.8 Hz, 3H), 2.55 (q, J=7.2 Hz, 2H), 1.03 (t, J=7.2 Hz, 3H).

### Example 15: 5-(4-((3-Ethyl-2-oxo-2,3-dihydro-1H-pyrimido[4,5,6-de]quinazolin-8-yl)methyl)piperazin-1-yl)-N-methylpicolinamide (I-15)

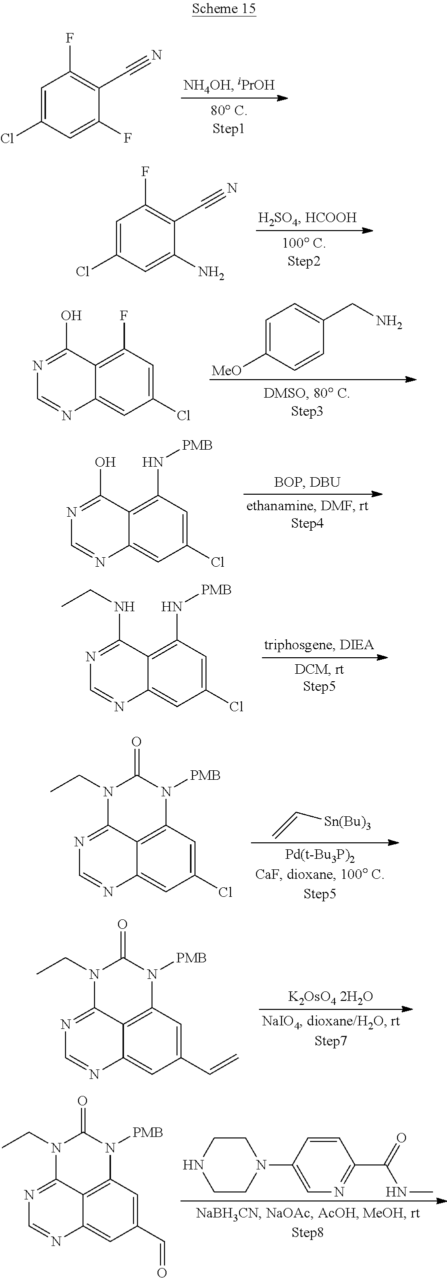

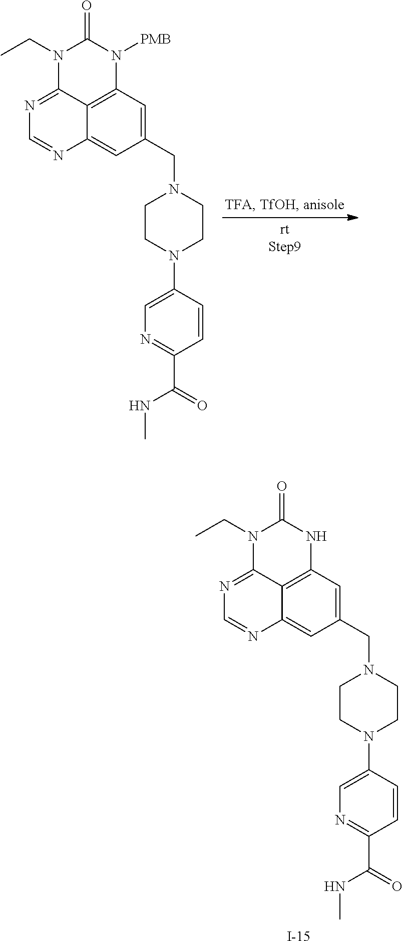

### Step 1:2-Amino-4-chloro-6-fluorobenzonitrile

The mixture of 4-chloro-2,6-difluorobenzonitrile (2 g, 11.5 mmol) and aqueous ammonia (28%, 11.22 mL) in isopropanol (5 mL) was stirred at 80° C. for 18 h. Upon cooling to room temperature, the mixture was poured into water (300 mL) and stirred for 15 min. The precipitated solids were collected by filtration, washed with toluene (2×50 mL) and dried under vacuum to provide the desired product as a white solid (2.2 g). LCMS calculated for C₇H₅ClFN₂ (M+H)⁺ m/z=171.0; found 171.0; ¹H NMR (300 MHz, CDCl₃) δ 6.57-6.56 (m, 1H), 6.55-6.52 (m, 1H), 4.65 (s, 2H).

### Step 2: 7-Chloro-5-fluoroquinazolin-4-ol

The mixture of 2-amino-4-chloro-6-fluorobenzonitrile (2 g, 11.7 mmol) in formic acid (40 mL) and sulfuric acid (98%, 3.2 mL) was stirred 100° C. for 2 h. Upon cooling to 0° C., the mixture was diluted with water (80 mL). The resulting suspension was stirred for 10 minutes then filtered. The solid was washed sequentially with water/isopropanol (16 mL, 1/1), isopropanol/2-methoxy-2-methylpropane (16 mL, 1/1) then 2-methoxy-2-methylpropane (16 mL). The solid was air dried for 10 minutes then dried in a vacuum to provide the desired product as a white solid (1.4 g, 60%). ¹H NMR (400 MHz, DMSO-d₆) δ 12.48 (s, 1H), 8.14 (s, 1H), 7.56-7.55 (m, 1H), 7.49 (dd, J=10.8, 2.0 Hz, 1H).

### Step 3: 7-Chloro-5-{[(4-methoxyphenyl)methyl]amino}quinazolin-4-ol

The mixture of 7-chloro-5-fluoroquinazolin-4-ol (1.2 g, 6.04 mmol) and (4-methoxyphenyl) methanamine (4.14 g, 30.22 mmol) in dimethyl sulfoxide (18 mL) was stirred at 80° C. for 2 h. Upon cooling to room temperature, the mixture was diluted with water (50 mL), ethyl acetate (200 mL) and the two phases separated. The aqueous phase was extracted with ethyl acetate (100 mL). The organic phases were combined, dried with anhydrous sodium sulfate, filtered, and concentrated under reduced pressure. The residue was purified by silica gel column chromatography, eluted with eluted with 10% methanol in dichloromethane to provide the desired product as a white solid (1 g, 52%). LCMS calculated for C₁₆H₁₅ClN₃O₂ (M+H)⁺ m/z=316.1; found 316.0; ¹H NMR (400 MHz, DMSO-d₆) δ 12.17 (s, 1H), 9.09 (t, J=5.6 Hz, 1H), 7.98 (s, 1H), 7.32-7.29 (m, 2H), 6.95-6.91 (m, 2H), 6.69 (d, J=2.0 Hz, 1H), 6.50 (d, J=2.0 Hz, 1H), 4.38 (d, J=5.2 Hz, 2H), 3.74 (s, 3H).

### Step 4: 7-Chloro-N⁴-ethyl-N⁵-(4-methoxybenzyl) quinazoline-4,5-diamine

To the mixture of 7-chloro-5-{[(4-methoxyphenyl)methyl]amino}quinazolin-4-ol (800 mg, 2.5 mmol) in N,N-dimethylformamide (5 mL) were added 1H-benzotriazol-1-yloxytris(dimethylamino) phosphonium hexafluorophosphate (3362 mg, 7.6 mmol) and 1,8-diazabicyclo[5.4.0]undec-7-ene (1273 mg, 8.36 mmol); and the mixture was stirred for 15 min, followed by the addition of ethylamine (571 mg, 12.67 mmol). The resulting mixture was stirred at room temperature for additional 18 h; and then purified directly by reverse-phase flash chromatography (column, C18 silica gel; mobile phase, acetonitrile in water (0.1% formic acid), 10% to 60% gradient over 22 min). The fractions were collected, combined and lyophilized to provide the desired product as a yellow solid (240 mg, 28%). LCMS calculated for C₁₈H₂₀ClN₄O (M+H)⁺ m/z=343.1; found 343.0.

### Step 5: 8-Chloro-3-ethyl-1-(4-methoxybenzyl)-1H-pyrimido[4,5,6-de]quinazolin-2(3H)-one

To the mixture of 7-chloro-N⁴-ethyl-N⁵-(4-methoxybenzyl) quinazoline-4,5-diamine (200 mg, 0.58 mmol) and N-ethyl-N-isopropylpropan-2-amine (754 mg, 5.8 mmol) in dichloromethane (10 mL) was added triphosgene (173 mg, 0.58 mmol) at 0° C. The mixture was allowed to stir at room temperature for 2 h; and then quenched with saturated aqueous ammonium chloride (20 mL), extracted with dichloromethane (3×20 mL). The organic layers were dried with anhydrous sodium sulfate. After filtration, the filtrate was concentrated under reduced pressure. The residue was purified by silica gel column chromatography, eluted with 20% ethyl acetate in petroleum ether to provide the desired product as a white solid (120 mg, 56%). LCMS calculated for C₁₉H₁₈ClN₄O₂ (M+H)⁺ m/z=369.1; found 369.0; ¹H NMR (400 MHz, CDCl₃) δ 8.78 (s, 1H), 7.44 (d, J=1.6 Hz, 1H), 7.28-7.25 (m, 2H), 6.90-6.87 (m, 2H), 6.83 (d, J=1.6 Hz, 1H), 5.22 (s, 2H), 4.39 (q, J=7.2 Hz, 2H), 3.79 (s, 3H), 1.38 (t, J=7.2 Hz, 3H).

### Step 6:3-Ethyl-1-(4-methoxybenzyl)-8-vinyl-1H-pyrimido[4,5,6-de]quinazolin-2(3H)-one

The mixture of 8-chloro-3-ethyl-1-(4-methoxybenzyl)-1H-pyrimido[4,5,6-de]quinazolin-2(3H)-one (110 mg, 0.3 mmol), tributyl(vinyl) stannane (142 mg, 0.45 mmol), cesium fluoride (100 mg, 0.66 mmol) and bis(tri-tert-butylphosphine)palladium(0) (15 mg, 0.03 mmol) in 1,4-dioxane (3 mL) was stirred at 100° C. for 1 h under nitrogen atmosphere. Upon cooling to room temperature, the mixture was concentrated under reduced pressure. The residue was purified by silica gel column chromatography, eluted with 20% ethyl acetate in petroleum ether to provide the desired product as a white solid (80 mg, 74%). LCMS calculated for C₂₁H₂₁N₄O₂ (M+H)⁺ m/z=361.2; found 361.0.

### Step 7:3-Ethyl-1-(4-methoxybenzyl)-2-oxo-2,3-dihydro-1H-pyrimido[4,5,6-de]quinazoline-8-carbaldehyde

The mixture of 3-ethyl-1-(4-methoxybenzyl)-8-vinyl-1H-pyrimido[4,5,6-de]quinazolin-2(3H)-one (70 mg, 0.19 mmol) in 1,4-dioxane (3 mL) and water (1 mL) was treated with potassium osmate (VI) dihydrate (14 mg, 0.04 mmol) and sodium metaperiodate (166 mg, 0.78 mmol); The reaction was stirred at the same temperature for additional 8 h; and then diluted with ethyl acetate (50 mL). The organic layers were washed with water (2×20 mL) and dried with anhydrous sodium sulfate. After filtration, the filtrate was concentrated under reduced pressure, and the residue was purified by silica gel column chromatography, eluted with 50% ethyl acetate in petroleum ether to provide the desired product as a yellow solid (40 mg, 57%). LCMS calculated for C₂₀H₁₉N₄O₃ (M+H)⁺ m/z=363.1; found 363.0.

### Step 8: 5-(4-((3-Ethyl-1-(4-methoxybenzyl)-2-oxo-2,3-dihydro-1H-pyrimido[4,5,6-de]quinazolin-8-yl)methyl)piperazin-1-yl)-N-methylpicolinamide

The mixture of 3-ethyl-1-(4-methoxybenzyl)-2-oxo-2,3-dihydro-1H-pyrimido[4,5,6-de]quinazoline-8-carbaldehyde (35 mg, 0.1 mmol), N-methyl-5-(piperazin-1-yl)picolinamide dihydrochloride (28 mg, 0.1 mmol), sodium acetate (47 mg, 0.49 mmol) in methanol (2 mL) was stirred at room temperature for 1 h; followed by the addition of sodium cyanoborohydride (12 mg, 0.19 mmol) and acetic acid (12 mg, 0.19 mmol). The resulting mixture was stirred at the same temperature for additional 1 h; and then concentrated under vacuum and the residue was purified by silica gel column chromatography, eluted with 10% methanol in dichloromethane to provide the desired product as a white solid (40 mg, 73%). LCMS calculated for C₃₁H₃₅N₈O₃ (M+H)⁺ m/z=567.3; found 567.2.

### Step 9: 5-(4-((3-Ethyl-2-oxo-2,3-dihydro-1H-pyrimido[4,5,6-de]quinazolin-8-yl)methyl)piperazin-1-yl)-N-methylpicolinamide (I-15)

The mixture of 5-(4-((3-ethyl-1-(4-methoxybenzyl)-2-oxo-2,3-dihydro-1H-pyrimido[4,5,6-de]quinazolin-8-yl)methyl)piperazin-1-yl)-N-methylpicolinamide (40 mg, 0.07 mmol) in 2,2,2-trifluoroacetic acid (5 mL) were treated with trifluoromethanesulfonic acid (0.5 mL) and anisole (0.3 mL) at room temperature; the reaction was stirred at the same temperature for 18 h; and then concentrated under reduced pressure. The residue was purified by reverse flash chromatography (column: Xselect CSH C₁₈ OBD column 30*150 mm 5 um; mobile phase A: water (0.05% trifluoroacetic acid), mobile phase B: acetonitrile; flow rate: 60 mL/min; gradient: 5% B to 29% B over 7 min); eluted fractions were collected and lyophilized to give the TFA salt of the desired product as a white solid (5.5 mg). LCMS calculated for C₂₃H₂₇N₈O₂ (M+H)⁺ m/z=447.2; found 447.1; ¹H NMR (400 MHz, DMSO-d₆) & 11.76 (s, 1H), 10.18 (s, 1H), 8.77 (s, 1H), 8.46-8.42 (m, 1H), 8.34 (d, J=2.8 Hz, 1H), 7.89 (d, J=8.8 Hz, 1H), 7.49-7.46 (m, 2H), 6.98 (s, 1H), 4.53 (s, 2H), 4.15 (q, J=7.2 Hz, 2H), 4.10-4.03 (m, 2H), 3.51-3.16 (m, 6H), 2.79 (d, J=4.8 Hz, 3H), 1.23 (t, J=7.2 Hz, 3H).

### Example 16: 5-(4-((3-ethyl-2,4-dioxo-1,2,3,4-tetrahydroquinazolin-7-yl)methyl)piperazin-1-yl)-N,6-dimethylpicolinamide (I-16)

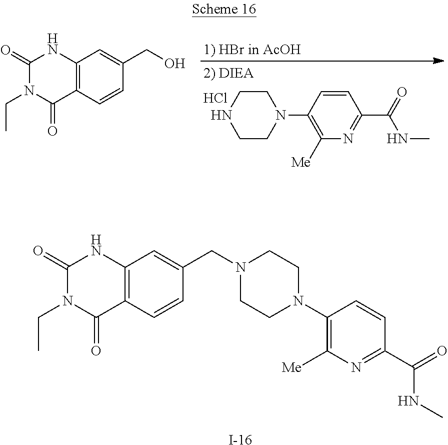

To 3-ethyl-7-(hydroxymethyl) quinazoline-2,4(1H,3H)-dione (see Example 1, step 2, 97 mg, 0.44 mmol) was added hydrogen bromide (33 wt. % solution in glacial acid, 3 mL) at room temperature; the reaction mixture was then heated at 80° C. under nitrogen atmosphere for 2 h. Upon cooling to room temperature, the reaction mixture was concentrated under reduced pressure. The residue was taken in acetonitrile (3 mL) followed by addition of N,6-dimethyl-5-(piperazin-1-yl)pyridine-2-carboxamide hydrochloride (130 mg, 0.48 mmol) and N-ethyl-N-isopropylpropan-2-amine (359 mg, 2.8 mmol). Then the resulted mixture was heated at 70° C. for additional 2 h. The mixture was allowed to cool to room temperature and concentrated under reduced pressure. The crude product was purified by reverse flash chromatography (column: Xselect CSH C18 OBD Column 30*150 mm 5 um; mobile phase A: water (0.05% trifluoroacetic acid), mobile phase B: acetonitrile; flow rate: 60 mL/min; gradient: 10% B to 35% B over 7 min); eluted fractions were collected and lyophilized to give the TFA salt of the desired product as a white solid (78.2 mg). LCMS calculated for C₂₃H₂₉N₆O₃ (M+H)⁺ m/z=437.2; found 437.2; ¹H NMR (400 MHz, DMSO-d₆) δ 11.72 (s, 1H), 10.03 (s, 1H), 8.48-8.44 (m, 1H), 8.06 (d, J=8.0 Hz, 1H), 7.83 (d, J=8.4 Hz, 1H), 7.56 (d, J=8.4 Hz, 1H), 7.36 (d, J=8.4 Hz, 1H), 7.31 (s, 1H), 4.52 (s, 2H), 3.95 (q, J=7.2 Hz, 2H), 3.42-3.34 (m, 6H), 3.05-2.97 (m, 2H), 2.81 (d, J=4.8 Hz, 3H), 2.52 (s, 3H), 1.16 (t, J=7.2 Hz, 3H).

### Example 17: 5-(4-((3-Ethyl-2-oxo-2,3-dihydro-1H-pyrimido[4,5,6-de]quinazolin-8-yl)methyl)piperazin-1-yl)-N,6-dimethylpicolinamide (I-17)

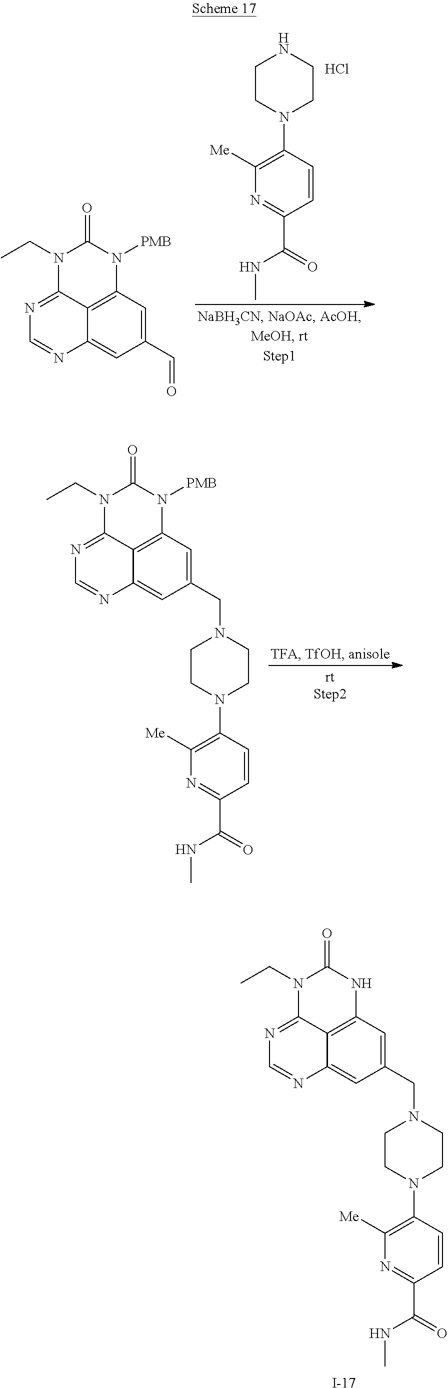

### Step 1: 5-(4-((3-Ethyl-1-(4-methoxybenzyl)-2-oxo-2,3-dihydro-1H-pyrimido[4,5,6-de]quinazolin-8-yl)methyl)piperazin-1-yl)-N,6-dimethylpicolinamide

The mixture of 3-ethyl-1-(4-methoxybenzyl)-2-oxo-2,3-dihydro-1H-pyrimido[4,5,6-de]quinazoline-8-carbaldehyde (Example 15, step 7, 40 mg, 0.1 mmol), sodium acetate (45 mg, 0.55 mmol) and N,6-dimethyl-5-(piperazin-1-yl)pyridine-2-carboxamide hydrochloride (45 mg, 0.16 mmol) in methanol (3 mL) was stirred at room temperature for 30 min, followed by the addition of acetic acid (20 mg, 0.33 mmol) and sodium cyanoborohydride (14 mg, 0.22 mmol). The resulting mixture was stirred for additional 1 h, then concentrated under vacuum and the residue was purified by silica gel column chromatography, eluted with 10% methanol in dichloromethane to provide the desired product as a white solid (30 mg, 47%). LCMS calculated for C₃₂H₃₇N₈O₃ (M+H)⁺ m/z=581.3; found 581.4.

### Step 2: 5-(4-((3-Ethyl-2-oxo-2,3-dihydro-1H-pyrimido[4,5,6-de]quinazolin-8-yl)methyl)piperazin-1-yl)-N,6-dimethylpicolinamide (I-17)

The mixture of 5-(4-((3-ethyl-1-(4-methoxybenzyl)-2-oxo-2,3-dihydro-1H-pyrimido[4,5,6-de]quinazolin-8-yl)methyl)piperazin-1-yl)-N,6-dimethylpicolinamide (30 mg, 0.05 mmol) in 2,2,2-trifluoroacetic acid (3 mL) were treated with anisole (0.6 mL) and trifluoromethanesulfonic acid (0.6 mL) at room temperature. The reaction mixture was stirred at the same temperature for 18 h, and then concentrated under reduced pressure. The residue was purified by reverse flash chromatography (column: XBridge Prep C18 OBD Column, 30*100 mm, 5 um; mobile phase A: water (10 mmol/L ammonium bicarbonate), mobile phase B: acetonitrile; flow rate: 60 mL/min; gradient: 22% B to 48% B in 7 min); eluted fractions were collected and concentrated under vacuum. The residue was lyophilized with water (0.05% 2,2,2-trifluoroacetic acid) and acetonitrile to give the TFA salt of the desired product as a white solid. LCMS calculated for C₂₄H₂₉N₈O₂ (M+H)⁺ m/z=461.2; found 461.2; ¹H NMR (400 MHz, CD₃OD) δ 8.83 (s, 1H), 7.90 (d, J=8.0 Hz, 1H), 7.57 (d, J=8.4 Hz, 1H), 7.53 (s, 1H), 7.14 (s, 1H), 4.59 (s, 2H), 4.33 (q, J=7.2 Hz, 2H), 3.56-3.49 (m, 4H), 3.29-3.24 (m, 4H), 2.94 (s, 3H), 2.59 (s, 3H), 1.34 (t, J=7.2 Hz, 3H).

### Example 18: 5-(4-((3-Ethyl-2-oxo-2,3-dihydro-1H-pyrimido[4,5,6-de]quinazolin-8-yl)methyl)piperazin-1-yl)-N-methyl-6-(trifluoromethyl)picolinamide (I-18)

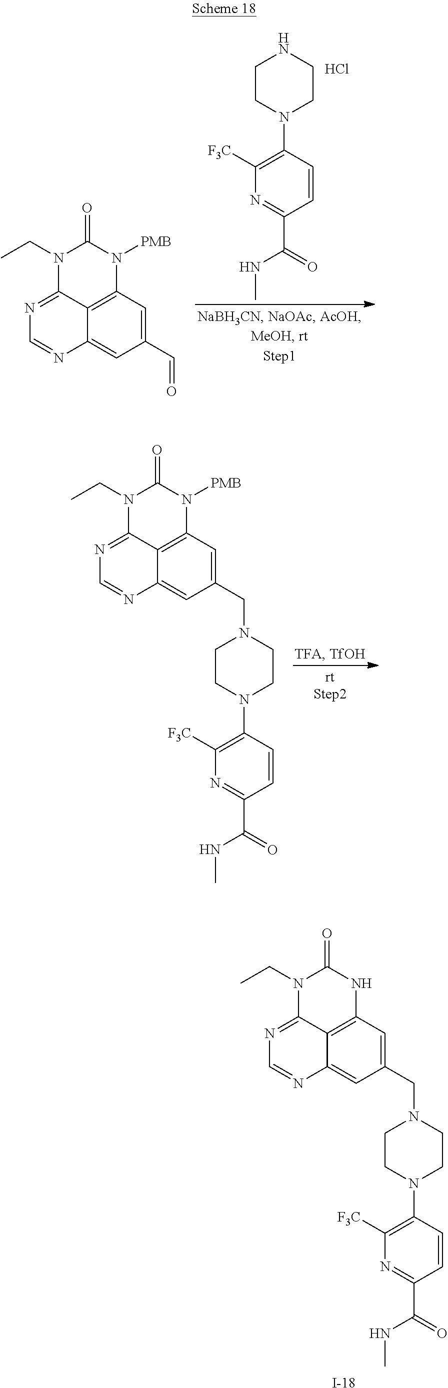

### Step 1: 5-(4-((3-Ethyl-1-(4-methoxybenzyl)-2-oxo-2,3-dihydro-1H-pyrimido[4,5,6-de]quinazolin-8-yl)methyl)piperazin-1-yl)-N-methyl-6-(trifluoromethyl)picolinamide

The mixture of 3-ethyl-1-(4-methoxybenzyl)-2-oxo-2,3-dihydro-1H-pyrimido[4,5,6-de]quinazoline-8-carbaldehyde (Example 15, step 7, 40 mg, 0.1 mmol), sodium acetate (45 mg, 0.55 mmol) and N-methyl-5-(piperazin-1-yl)-6-(trifluoromethyl)pyridine-2-carboxamide hydrogen chloride (54 mg, 0.16 mmol) in methanol (3 mL) was stirred at room temperature for 30 min, followed by the addition of acetic acid (20 mg, 0.33 mmol) and sodium cyanoborohydride (14 mg, 0.22 mmol). The resulting mixture was stirred for additional 1 h and then concentrated under vacuum. The residue was purified by silica gel column chromatography, eluted with 10% methanol in dichloromethane to provide the desired product as a white solid (18 mg, 26%). LCMS calculated for C₃₂H₃₄F₃N₈O₃ (M+H)⁺ m/z=635.3; found 635.2; ¹H NMR (400 MHz, DMSO-d₆) δ 8.72 (s, 1H), 8.43-8.39 (m, 1H), 8.21 (d, J=8.4 Hz, 1H), 7.99 (d, J=8.8 Hz, 1H), 7.34-7.30 (m, 3H), 7.00 (s, 1H), 6.89 (d, J=7.2 Hz, 2H), 5.26 (s, 2H), 4.25 (q, J=7.2 Hz, 2H), 3.67 (s, 2H), 3.63 (s, 3H), 2.90-2.88 (m, 4H), 2.83 (d, J=4.8 Hz, 3H), 2.45-2.42 (m, 4H), 1.27 (t, J=7.2 Hz, 3H).

### Step 2: 5-(4-((3-Ethyl-2-oxo-2,3-dihydro-1H-pyrimido[4,5,6-de]quinazolin-8-yl)methyl)piperazin-1-yl)-N-methyl-6-(trifluoromethyl)picolinamide (I-18)

To a solution of 5-(4-((3-ethyl-1-(4-methoxybenzyl)-2-oxo-2,3-dihydro-1H-pyrimido[4,5,6-de]quinazolin-8-yl)methyl)piperazin-1-yl)-N-methyl-6-(trifluoromethyl)picolinamide (18 mg, 0.03 mmol) in 2,2,2-trifluoroacetic acid (3 mL) was added trifluoromethanesulfonic acid (0.6 mL). The mixture was stirred at the room temperature for 18 h, and then concentrated under reduced pressure. The residue was purified by reverse-phase prep HPLC (column: SunFire Prep C18 OBD column 30*150 mm 5 um; mobile phase A: water (0.05% trifluoroacetic acid), mobile phase B: acetonitrile; flow rate: 60 mL/min); eluted fractions were collected and lyophilized to give the TFA salt of the desired product as a white solid (6.0 mg). LCMS calculated for C₂₄H₂₆F₃N₈O₂ (M+H)⁺ m/z=515.2; found 515.2. ¹H NMR (400 MHz, CD₃OD) δ 8.79 (s, 1H), 8.30 (d, J=8.4 Hz, 1H), 8.06 (d, J=8.4 Hz, 1H), 7.50 (d, J=1.2 Hz, 1H), 8.10 (d, J=1.2 Hz, 1H), 4.55 (s, 2H), 4.31 (q, J=7.2 Hz, 2H), 3.50-3.45 (m, 4H), 3.34-3.32 (m, 4H), 2.97 (s, 3H), 1.33 (t, J=7.2 Hz, 3H).

### Example 19: 5-(4-((3-ethyl-2-oxo-2,3-dihydro-1H-pyrimido[4,5,6-de]quinazolin-8-yl)methyl)piperazin-1-yl)-6-fluoro-N-methylpicolinamide (I-19)

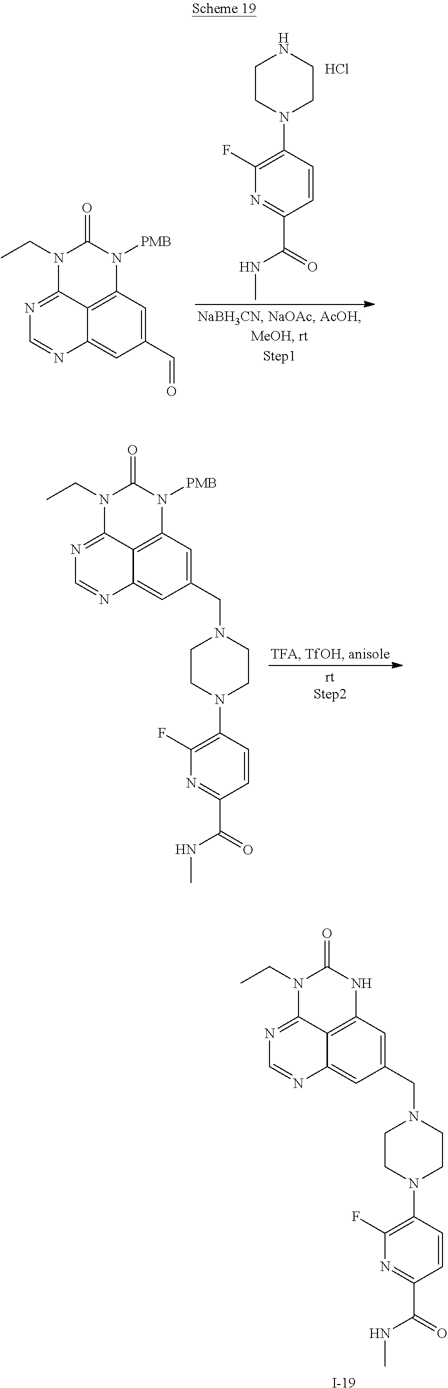

### Step 1: 5-(4-((3-ethyl-1-(4-methoxybenzyl)-2-oxo-2,3-dihydro-1H-pyrimido[4,5,6-de]quinazolin-8-yl)methyl)piperazin-1-yl)-6-fluoro-N-methylpicolinamide

The mixture of 3-ethyl-1-(4-methoxybenzyl)-2-oxo-2,3-dihydro-1H-pyrimido[4,5,6-de]quinazoline-8-carbaldehyde (Example 15, step 7, 40 mg, 0.1 mmol), sodium acetate (45 mg, 0.55 mmol) and 6-fluoro-N-methyl-5-(piperazin-1-yl)picolinamide hydrochloride (45 mg, 0.16 mmol) in in methanol (3 mL) was stirred at room temperature for 30 min, followed by the addition of acetic acid (20 mg, 0.33 mmol) and sodium cyanoborohydride (14 mg, 0.22 mmol). The resulting mixture was stirred for additional 1 h, and then concentrated under vacuum. The residue was purified by silica gel column chromatography, eluted with 10% methanol in dichloromethane to provide the desired product as a white solid (30 mg, 46%). LCMS calculated for C₃₁H₃₄FN₈O₃ (M+H)⁺ m/z=585.3; found 585.4.

### Step 2: 5-(4-((3-ethyl-2-oxo-2,3-dihydro-1H-pyrimido[4,5,6-de]quinazolin-8-yl)methyl)piperazin-1-yl)-6-fluoro-N-methylpicolinamide (I-19)

The mixture of 5-(4-((3-ethyl-1-(4-methoxybenzyl)-2-oxo-2,3-dihydro-1H-pyrimido[4,5,6-de]quinazolin-8-yl)methyl)piperazin-1-yl)-6-fluoro-N-methylpicolinamide (30 mg, 0.05 mmol) in 2,2,2-trifluoroacetic acid (3 mL) were treated with anisole (0.6 mL) and trifluoromethanesulfonic acid (0.6 mL) at room temperature. The reaction was stirred at the same temperature for 18 h, concentrated under reduced pressure, and diluted with dichloromethane (20 mL). The pH value was basified to pH 8 with saturated sodium bicarbonate. The resulting mixture was extracted with dichloromethane (3×20 mL). The organic layers were dried with anhydrous sodium sulfate, filtered, and concentrated under reduced pressure. The residue was purified by silica gel column chromatography, eluted with 10% methanol in dichloromethane. The fractions were concentrated. The residue was lyophilized with water (0.05% 2,2,2-trifluoroacetic acid) and acetonitrile to provide the TFA salt of the desired product as a white solid (10 mg). LCMS calculated for C₂₃H₂₆FN₈O₂ (M+H)⁺ m/z=465.2; found 465.3; ¹H NMR (300 MHz, CD₃OD) δ 8.85 (s, 1H), 7.96 (dd, J=8.1, 1.2 Hz, 1H), 7.67-7.60 (m, 1H), 7.53 (d, J=1.5 Hz, 1H), 7.14 (d, J=1.2 Hz, 1H), 4.57 (s, 2H), 4.35 (q, J=6.9 Hz, 2H), 3.57-3.47 (m, 8H), 2.93 (s, 3H), 1.36 (t, J=6.9 Hz, 3H).

### Example 20: N,6-dimethyl-5-(4-((6-oxo-6,7,8,9-tetrahydro-5H-cyclopenta[c][1,5]naphthyridin-3-yl)methyl)piperazin-1-yl)picolinamide (I-20)

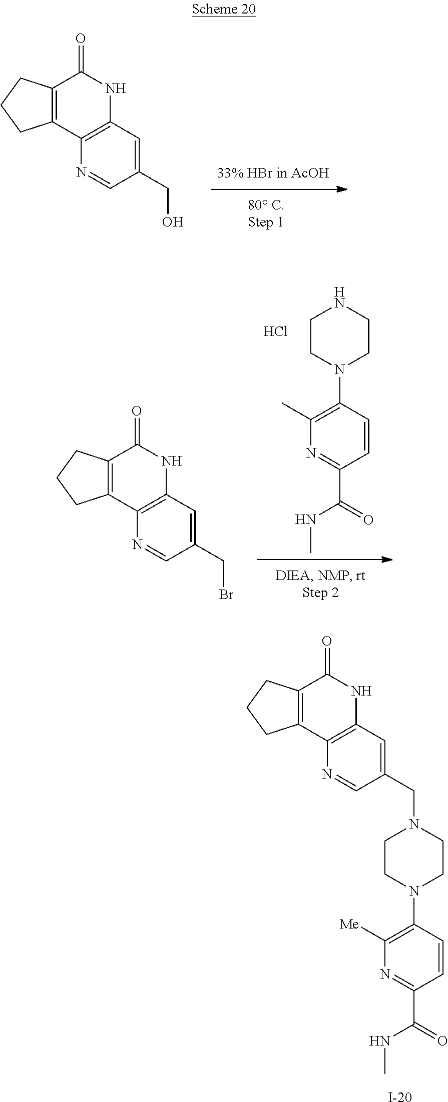

### Step 1:3-(bromomethyl)-5,7,8,9-tetrahydro-6H-cyclopenta[c][1,5]naphthyridin-6-one

The mixture of 3-(hydroxymethyl)-5,7,8,9-tetrahydro-6H-cyclopenta[c][1,5]naphthyridin-6-one (Example 6, step 4, 40 mg, 0.19 mmol) combined with hydrogen bromide (33 wt. % solution in glacial acid, 1 mL) at room temperature; the reaction mixture was then heated at 80° C. under nitrogen atmosphere for 2 h. Upon cooling to room temperature, the reaction mixture was concentrated under reduced pressure to provide the desired product as a yellow solid (50 mg, crude). LCMS calculated for C₁₂H₁₂BrN₂O (M+H)⁺ m/z=279.0; found 279.0, 281.0.

### Step 2: N,6-dimethyl-5-(4-((6-oxo-6,7,8,9-tetrahydro-5H-cyclopenta[c][1,5]naphthyridin-3-yl)methyl)piperazin-1-yl)picolinamide (I-20)

The mixture of 3-(bromomethyl)-5,7,8,9-tetrahydro-6H-cyclopenta[c][1,5]naphthyridin-6-one (24 mg, 0.1 mmol), N-ethyl-N-isopropylpropan-2-amine (88 mg, 0.7 mmol) and N,6-dimethyl-5-(piperazin-1-yl)pyridine-2-carboxamide hydrochloride (23 mg, 0.1 mmol) in 1-methylpyrrolidin-2-one (0.5 mL) was stirred at room temperature for 4 h. The crude product was purified by reverse flash chromatography (column: Xselect CSH C₁₈ OBD Column 30*150 mm 5 um; mobile phase A: water (0.05% trifluoroacetic acid), mobile phase B: acetonitrile; flow rate: 60 mL/min; gradient: 10% B to 25% B over 7 min); eluted fractions were collected and lyophilized to provide the TFA salt of the desired product as a white solid (3.8 mg). LCMS calculated for C₂₄H₂₉N₆O₂ (M+H)⁺ m/z=433.2; found 433.1; ¹H NMR (300 MHz, DMSO-d₆) δ 12.06 (s, 1H), 9.99 (s, 1H), 8.59 (d, J=1.8 Hz, 1H), 8.49-8.44 (m, 1H), 7.85-7.83 (m, 2H), 7.57 (d, J=8.4 Hz, 1H), 4.59 (s, 2H), 3.52-3.30 (m, 6H), 3.22-3.17 (m, 2H), 3.05-2.96 (m, 2H), 2.88-2.80 (m, 5H), 2.53 (s, 3H), 2.20-2.10 (m, 2H).

### Example 21: 5-(4-((3-ethyl-8-fluoro-2,4-dioxo-1,2,3,4-tetrahydroquinazolin-7-yl)methyl)piperazin-1-yl)-N,6-dimethylpicolinamide (I-21)

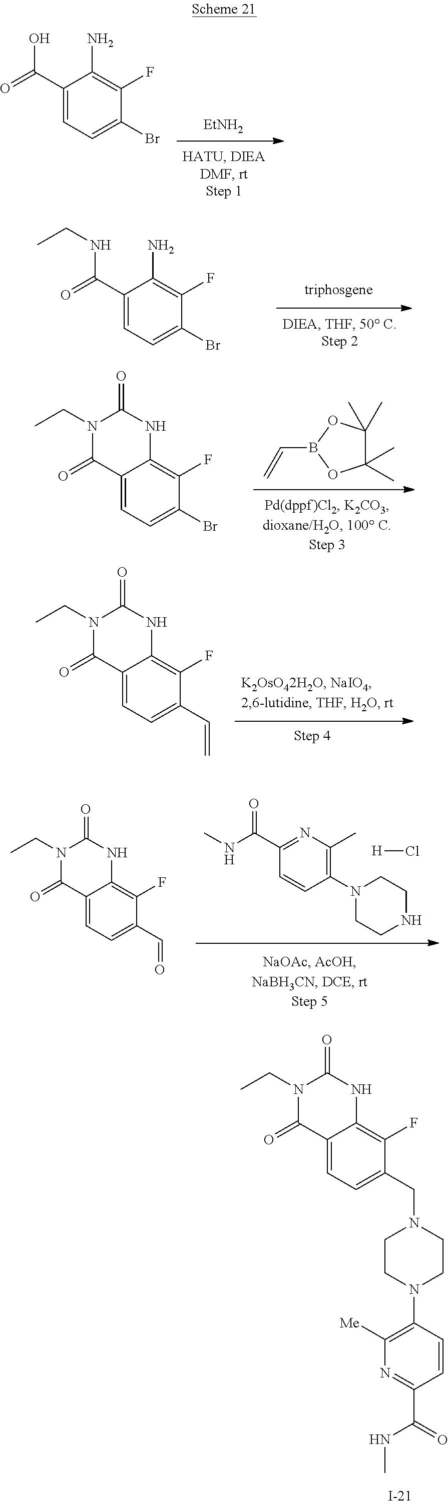

### Step 1:2-Amino-4-bromo-N-ethyl-3-fluorobenzamide

The mixture of 2-amino-4-bromo-3-fluorobenzoic acid (5 g, 21.37 mmol) in N,N-dimethylformamide (50 mL) was treated with N,N,N,N-tetramethyl-O-(7-azabenzotriazol-1-yl) uronium hexafluorophospate (9.75 g, 25.64 mmol) at room temperature for 20 min, followed by the addition of ethylamine (2 M in tetrahydrofuran, 16 mL, 32 mmol) and N-ethyl-N-isopropylpropan-2-amine (5.52 g, 42.73 mmol). The resulting mixture was stirred at the same temperature for 16 h, and then diluted with water (300 mL). The aqueous layer was extracted with ethyl acetate (3×100 mL). The combined organics were dried with anhydrous sodium sulfate. After filtered, the filtrate was concentrated under reduced pressure. The residue was purified by silica gel column chromatography, eluted with 60% ethyl acetate in petroleum ether to provide the desired product as a white solid (3.4 g, 61%). LCMS calculated for C₉H₁₁BrFN₂O (M+H)⁺ m/z=261.0; found 261.0.

### Step 2: 7-Bromo-3-ethyl-8-fluoroquinazoline-2,4(1H,3H)-dione

The mixture of 2-amino-4-bromo-N-ethyl-3-fluorobenzamide (2.7 g, 10.34 mmol) in anhydrous tetrahydrofuran (50 mL) was treated with triphosgene (3.07 g, 10.34 mmol) and N-ethyl-N-isopropylpropan-2-amine (2.67 g, 20.68 mmol) at 0° C. The reaction mixture was stirred for 0.5 h at room temperature and then heated at 50° C. for 1 h. Upon cooling to room temperature, the mixture was concentrated under reduced pressure. The residue was purified by silica gel column chromatography, eluted with 20% ethyl acetate in dichloromethane to provide the desired product as a white solid (1.6 g, 54%). LCMS calculated for C₁₀H₇BrFN₂O₂ (M−H)⁻ m/z=285.0; found 285.1; ¹H NMR (300 MHz, DMSO-d₆) δ 11.75 (s, 1H), 7.69-7.66 (m, 1H), 7.48-7.43 (m, 1H), 3.92 (q, J=6.9 Hz, 2H), 1.15 (t, J=6.9 Hz, 3H).

### Step 3:3-Ethyl-8-fluoro-7-vinylquinazoline-2,4(1H,3H)-dione

The mixture of 7-bromo-3-ethyl-8-fluoroquinazoline-2,4(1H,3H)-dione (1.9 g, 6.62 mmol), 2-ethenyl-4,4,5,5-tetramethyl-1,3,2-dioxaborolane (1.53 g, 9.93 mmol, 1.5 equiv), 1,1′-bis(diphenylphosphino)ferrocene-palladium(II) dichloride dichloromethane complex (0.54 g, 0.66 mmol) and potassium carbonate (1.83 g, 13.24 mmol) in 1,4-dioxane (20 mL) and water (4 mL) was stirred at 80° C. for 2 h under nitrogen atmosphere. Upon cooling to room temperature, the mixture was concentrated under reduced pressure, and the residue was purified by silica gel column chromatography, eluted with 20% ethyl acetate in dichloromethane to provide the desired product as a white solid (1 g, 65%). LCMS calculated for C₁₂H₁₀FN₂O₂ (M−H)⁻ m/z=233.1; found 232.9; ¹H NMR (400 MHz, CDCl₃) δ 8.76 (s, 1H), 7.86 (d, J=8.4 Hz, 1H), 7.33-7.29 (m, 1H), 6.92 (dd, J=18.0, 11.2 Hz, 1H), 6.01 (d, J=18.0 Hz, 1H), 5.61 (d, J=11.2 Hz, 1H), 4.14 (q, J=7.2 Hz, 2H), 1.31 (t, J=7.2 Hz, 3H).

### Step 4:3-Ethyl-8-fluoro-2,4-dioxo-1,2,3,4-tetrahydroquinazoline-7-carbaldehyde

The mixture of 3-ethyl-8-fluoro-7-vinylquinazoline-2,4(1H,3H)-dione (950 mg, 4.06 mmol) and 2,6-lutidine (869 mg, 8.11 mmol) in 1,4-dioxane (10 mL) and water (2 mL) was treated with potassium osmate (VI) dihydrate (299 mg, 0.81 mmol) and sodium metaperiodate (3.47 g, 16.22 mmol) at room temperature for 2 h. The reaction mixture was then diluted with ethyl acetate (50 mL). The organic layers were washed with water (2×20 mL) and dried with anhydrous sodium sulfate. After filtration, the filtrate was concentrated under reduced pressure, and the residue was purified by silica gel column chromatography, eluted with 40% ethyl acetate in petroleum ether to provide the desired product as a white solid (800 mg, 84%). LCMS calculated for C₁₁H₈FN₂O₃ (M−H)⁻ m/z=235.1; found 235.0; ¹H NMR (400 MHz, DMSO-d₆) δ 11.87 (s, 1H), 10.29 (s, 1H), 7.89-7.87 (m, 1H), 7.56-7.52 (m, 1H), 3.95 (q, J=7.2 Hz, 2H), 1.17 (t, J=7.2 Hz, 3H).

### Step 5: 5-(4-((3-Ethyl-8-fluoro-2,4-dioxo-1,2,3,4-tetrahydroquinazolin-7-yl)methyl)piperazin-1-yl)-N,6-dimethylpicolinamide (I-21)

The mixture of 3-ethyl-8-fluoro-2,4-dioxo-1,2,3,4-tetrahydroquinazoline-7-carbaldehyde (26 mg, 0.11 mmol), N,6-dimethyl-5-(piperazin-1-yl)picolinamide hydrochloride (30 mg, 0.11 mmol) and sodium acetate (45 mg, 0.56 mmol) in 1,2-dichloroethane (1 mL) was stirred at room temperature for 20 min, followed by the addition of sodium cyanoborohydride (14 mg, 0.22 mmol) and acetic acid (13 mg, 0.22 mmol). The resulting mixture was stirred at room temperature for additional 16 h, which was then concentrated under vacuum. The residue was purified by reverse flash chromatography (column: Xbridge Shield RP18 OBD Column, 19*150 mm, 5 um; mobile phase A: water (10 mmol/L ammonium bicarbonate), mobile phase B: acetonitrile; flow rate: 25 mL/min; gradient: 25% B to 52% B over 7 min); eluted fractions were collected and concentration under vacuum. The residue was lyophilized with water (0.05% 2,2,2-trifluoroacetic acid) and acetonitrile to provide the TFA salt of the desired product as a white solid (2.3 mg). LCMS calculated for C₂₃H₂₈FN₆O₃ (M+H)⁺ m/z=455.2; found 455.3; ¹H NMR (400 MHz, DMSO-d₆) δ 11.81 (s, 1H), 8.48-8.44 (m, 1H), 7.89-7.82 (m, 2H), 7.55 (d, J=8.0 Hz, 1H), 7.42-7.38 (m, 1H), 4.54 (s, 2H), 3.96 (q, J=6.8 Hz, 2H), 3.46-3.11 (m, 8H), 2.81 (d, J=4.8 Hz, 3H), 2.52 (s, 3H), 1.17 (t, J=6.8 Hz, 3H).

### Example 22: 5-(4-((3-ethyl-5-fluoro-2,4-dioxo-1,2,3,4-tetrahydroquinazolin-7-yl)methyl)piperazin-1-yl)-N,6-dimethylpicolinamide (I-22)

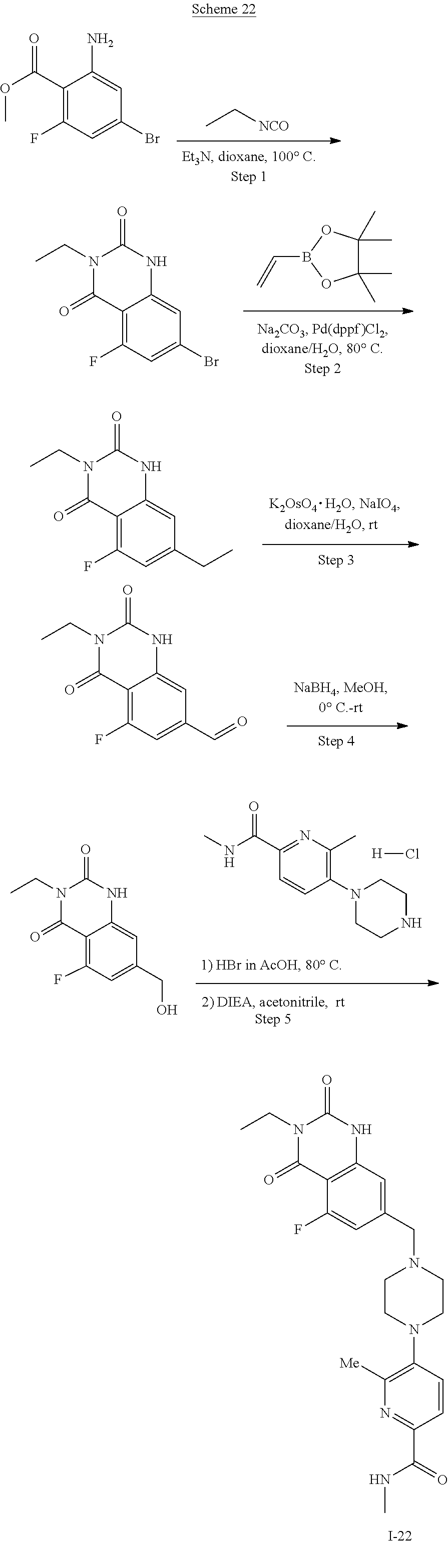

### Step 1: 7-Bromo-3-ethyl-5-fluoroquinazoline-2,4(1H,3H)-dione

To a mixture of methyl 2-amino-4-bromo-6-fluorobenzoate (4.5 g, 18.14 mmol) in 1,4-dioxane (70 mL) were added isocyanatoethane (3.87 g, 54.42 mmol) and triethylamine (9.18 g, 90.71 mmol) at 0° C. under nitrogen atmosphere. The resulting mixture was stirred at 100° C. for 16 h. Upon cooling to room temperature, the resulting mixture was concentrated under reduced pressure. The residue was purified by silica gel column chromatography, eluted with 40% ethyl acetate in petroleum to provide the desired product as a white solid (3.0 g, 58%). LCMS calculated for C₁₀H₉BrFN₂O₂ (M+H)⁺ m/z=287.0; found 286.9; ¹H NMR (400 MHz, CDCl₃) δ 10.36 (s, 1H), 7.10-7.06 (m, 2H), 4.13 (q, J=6.8 Hz, 2H), 1.31 (t, J=6.8 Hz, 3H).

### Step 2:3-Ethyl-5-fluoro-7-vinylquinazoline-2,4(1H,3H)-dione

The mixture of 7-bromo-3-ethyl-5-fluoroquinazoline-2,4(1H,3H)-dione (2.8 g, 9.75 mmol), 2-ethenyl-4,4,5,5-tetramethyl-1,3,2-dioxaborolane (2.25 g, 14.63 mmol), sodium carbonate (2.07 g, 19.51 mmol) and 1,1′-bis(diphenylphosphino)ferrocene-palladium(II) dichloride dichloromethane complex (0.79 g, 0.98 mmol) in 1,4-dioxane (50 mL) and water (10 mL) was stirred at 80° C. for 4 h under nitrogen atmosphere. Upon cooling to room temperature, the mixture was diluted with water (150 mL), and then extracted with ethyl acetate (3×200 mL). The combined organic layers were dried with anhydrous sodium sulfate. After filtration, the filtrate was concentrated under reduced pressure. The residue was purified by silica gel column chromatography, eluted with 50% ethyl acetate in dichloromethane to provide the desired product as a brown solid (2.7 g, crude). LCMS calculated for C₁₂H₁₂FN₂O₂ (M+H)⁺ m/z=235.1; found 235.1.

### Step 3:3-Ethyl-5-fluoro-2,4-dioxo-1,2,3,4-tetrahydroquinazoline-7-carbaldehyde

To a solution of 3-ethyl-5-fluoro-7-vinylquinazoline-2,4(1H,3H)-dione (2.5 g, 10.67 mmol) and sodium metaperiodate (6.85 g, 32.02 mmol) in 1,4-dioxane (40 mL) and water (8 mL) was added potassium osmate (VI) dihydrate (0.79 g, 2.14 mmol). The resulting mixture was stirred at room temperature for 4 h; and then diluted with ethyl acetate (100 mL). The organic layers were washed with water (2×50 mL) and dried with anhydrous sodium sulfate. After filtration, the filtrate was concentrated under reduced pressure, and the residue was purified by silica gel column chromatography, eluted with 40% ethyl acetate in dichloromethane to provide the desired product as a yellow solid (1.1 g, 44%). LCMS calculated for C₁₁H₈FN₂O₃ (M−H)⁻ m/z=235.1; found 234.8; ¹H NMR (300 MHz, CDCl₃) δ 10.11 (s, 1H), 9.40 (s, 1H), 7.48-7.44 (m, 2H), 4.23 (q, J=6.9 Hz, 2H), 1.42 (t, J=6.9 Hz, 3H).

### Step 4:3-Ethyl-5-fluoro-7-(hydroxymethyl) quinazoline-2,4(1H,3H)-dione

To a mixture of 3-ethyl-5-fluoro-2,4-dioxo-1,2,3,4-tetrahydroquinazoline-7-carbaldehyde (1 g, 4.23 mmol) in methanol (15 mL) was added sodium borohydride (0.64 g, 16.94 mmol) in portions at 0° C. The resulting mixture was stirred at room temperature for 1 h, and then quenched with water (5 mL). The resulting mixture was concentrated under reduced pressure. The residue was purified by silica gel column chromatography, eluted with 10% methanol in dichloromethane to provide the desired product as a grey solid (872 mg, 86%). LCMS calculated for C₁₁H₁₂FN₂O₃ (M+H)+m/z=239.1; found 239.0; ¹H NMR (400 MHz, DMSO-d₆) δ 11.52 (s, 1H), 6.98 (s, 1H), 6.85 (d, J=12.0 Hz, 1H), 5.51 (t, J=6.0 Hz, 1H), 4.53 (d, J=6.0 Hz, 2H), 3.88 (q, J=7.2 Hz, 2H), 1.13 (t, J=7.2 Hz, 3H).

### Step 5: 5-(4-((3-ethyl-5-fluoro-2,4-dioxo-1,2,3,4-tetrahydroquinazolin-7-yl)methyl)piperazin-1-yl)-N,6-dimethylpicolinamide (I-22)

The mixture of 3-ethyl-5-fluoro-7-(hydroxymethyl) quinazoline-2,4(1H,3H)-dione (300 mg, 1.26 mmol) in was combined with hydrogen bromide (33 wt. % solution in glacial acid, 5 mL) at room temperature. The reaction mixture was then heated at 80° C. under nitrogen atmosphere for 2 h. Upon cooling to room temperature, the reaction mixture was concentrated under reduced pressure. The residue was taken in acetonitrile (10 mL) followed by addition of N,6-dimethyl-5-(piperazin-1-yl)picolinamide hydrochloride (358 mg, 1.32 mmol) and N-ethyl-N-isopropylpropan-2-amine (1.3 g, 10.07 mmol). The resulting mixture was stirred at room temperature for additional 16 h, and then concentrated under reduced pressure. The residue was purified by silica gel column chromatography, eluted with 7% methanol in dichloromethane. The fractions were concentrated under vacuum. The residue was lyophilized with water (0.05% 2,2,2-trifluoroacetic acid) and acetonitrile to provide the TFA salt of the desired product as a white solid (415.4 mg). LCMS calculated for C₂₃H₂₈FN₆O₃ (M+H)⁺ m/z=455.2; found 455.1; ¹H NMR (400 MHz, CD₃OD) δ 7.89 (dd, J=8.4, 3.2 Hz, 1H), 7.55 (d, J=8.4 Hz, 1H), 7.14-7.10 (m, 2H), 4.42 (s, 2H), 4.04 (q, J=6.8 Hz, 2H), 3.49-3.41 (m, 4H), 3.28-3.23 (m, 4H), 2.94 (s, 3H), 2.58 (s, 3H), 1.27 (t, J=6.8 Hz, 3H).

### Example 23: N-methyl-5-(4-((6-oxo-6,7,8,9-tetrahydro-5H-cyclopenta[c][1,5]naphthyridin-3-yl)methyl)piperazin-1-yl)-6-(trifluoromethyl)picolinamide (I-23)

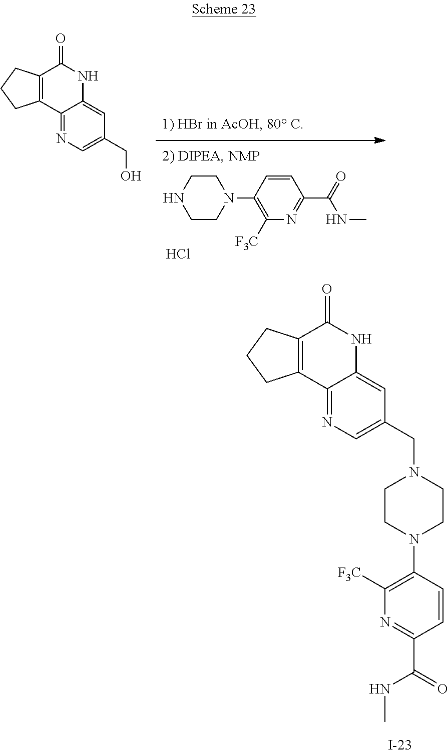

The mixture of 3-(hydroxymethyl)-5,7,8,9-tetrahydro-6H-cyclopenta[c][1,5]naphthyridin-6-one (Example 6, step 4, 25 mg, 0.12 mmol) was combined with hydrogen bromide (33 wt. % solution in glacial acid, 1 mL) at room temperature. The reaction mixture was then heated at 80° C. under nitrogen atmosphere for 2 h. Upon cooling to room temperature, the reaction mixture was concentrated under reduced pressure. The residue was taken in 1-methylpyrrolidin-2-one (0.8 mL) followed by addition of N-methyl-5-(piperazin-1-yl)-6-(trifluoromethyl)picolinamide hydrochloride (38 mg, 0.12 mmol) and N-ethyl-N-isopropylpropan-2-amine (231 mg, 1.80 mmol). The resulting mixture was stirred at room temperature for 16 h, and then purified by prep-HPLC (column: Sunfire prep C18 column, 30*150 mm, 5 um; mobile phase A: water (0.05% trifluoroacetic acid), mobile phase B: acetonitrile; flow rate: 60 mL/min; gradient: 8% B to 32% B in 7 min); eluted fractions were collected and lyophilized to provide the TFA salt of the desired product as a white solid (18.3 mg). LCMS calculated for C24H26F3N₆₀₂ (M+H)⁺ m/z=487.2; found 487.0; ¹H NMR (400 MHz, DMSO-d₆) δ 12.02 (s, 1H), 10.03 (s, 1H), 8.58 (s, 1H), 8.49-8.45 (m, 1H), 8.25 (d, J=8.4 Hz, 1H), 8.10 (d, J=8.4 Hz, 1H), 7.83 (s, 1H), 4.59 (s, 2H), 3.53-3.44 (m, 2H), 3.34-3.24 (m, 4H), 3.21-3.17 (m, 4H), 2.86-2.83 (m, 5H), 2.18-2.10 (m, 2H).

### Example 24: 6-Fluoro-N-methyl-5-(4-((6-oxo-6,7,8,9-tetrahydro-5H-cyclopenta[c][1,5]naphthyridin-3-yl)methyl)piperazin-1-yl)picolinamide (I-24)

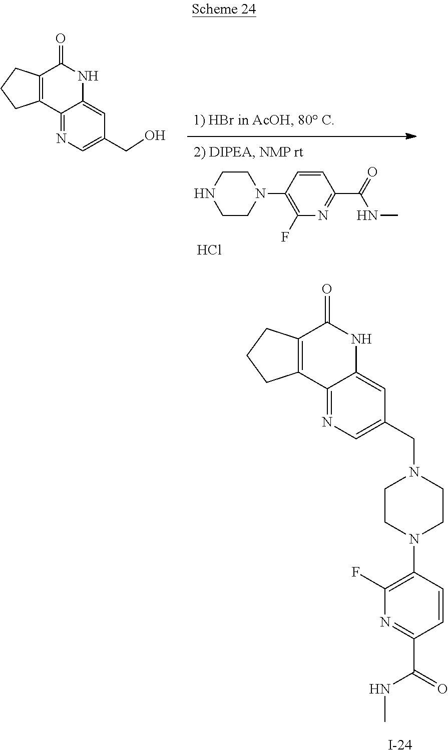

The mixture of 3-(hydroxymethyl)-5,7,8,9-tetrahydro-6H-cyclopenta[c][1,5]naphthyridin-6-one (Example 6, step 4, 20 mg, 0.09 mmol) was combined with hydrogen bromide (33 wt. % solution in glacial acid, 1 mL) at room temperature; the reaction was then heated at 80° C. under nitrogen atmosphere for 2 h. Upon cooling to room temperature, the reaction was concentrated under reduced pressure. The residue was taken in 1-methylpyrrolidin-2-one (0.8 mL) followed by addition of 6-fluoro-N-methyl-5-(piperazin-1-yl)picolinamide hydrochloride (25 mg, 0.09 mmol) and N-ethyl-N-isopropylpropan-2-amine (185 mg, 1.4 mmol). The resulting mixture was stirred at room temperature for 16 h and then purified by prep-HPLC (column: SunFire C18 OBD Prep Column, 19*250 mm, 5 um; Mobile Phase A: water (0.05% trifluoroacetic acid), Mobile Phase B: acetonitrile; flow rate: 60 mL/min; gradient: 8% B to 33% B in 8 min); eluted fractions were collected and lyophilized to provide the TFA salt of the desired product as a white solid (20.9 mg) LCMS calculated for C₂₃H₂₆FN₆O₂ (M+H)⁺ m/z=437.2; found 437.3; ¹H NMR (400 MHz, CD₃OD) δ 8.63 (d, J=1.2 Hz, 1H), 7.95-7.92 (m, 2H), 7.63-7.59 (m, 1H), 4.57 (s, 2H), 3.53-3.48 (m, 8H), 3.34-3.31 (m, 2H), 2.99-2.96 (m, 2H), 2.91 (s, 3H), 2.30-2.22 (m, 2H).

### Example 25: 5-(4-((4-Fluoro-6-oxo-6,7,8,9-tetrahydro-5H-cyclopenta[c][1,6]naphthyridin-3-yl)methyl)piperazin-1-yl)-N,6-dimethylpicolinamide (I-25)

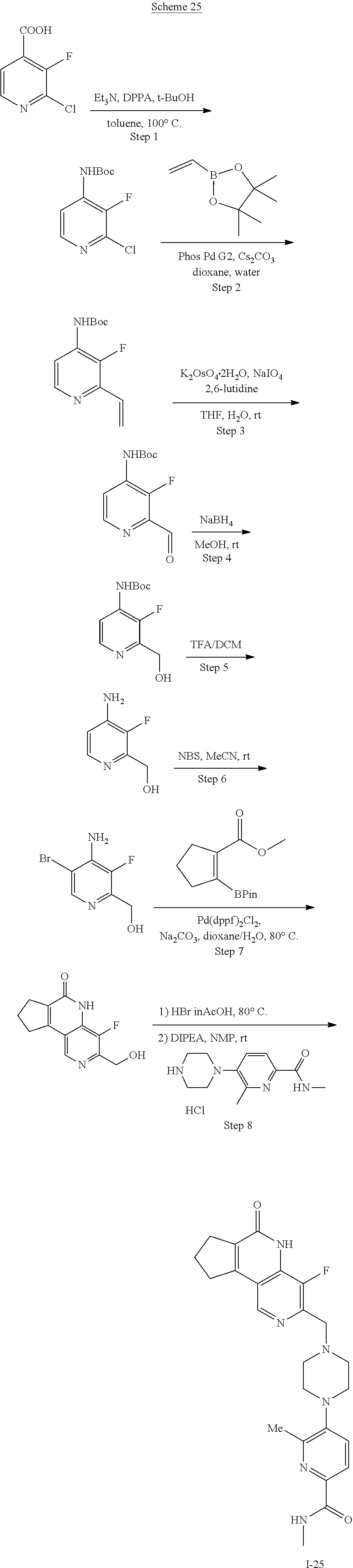

### Step 1: Tert-butyl(2-chloro-3-fluoropyridin-4-yl) carbamate

The mixture of 2-chloro-3-fluoroisonicotinic acid (10 g, 56.97 mmol), and trimethylamine (23.76 mL, 170.9 mmol) in dry toluene (70 mL) was treated with diphenylphosphoryl azide (23.52 g, 85.45 mmol) at 0° C. for 20 min under nitrogen atmosphere, followed by the addition of t-BuOH (70 mL) dropwise at the same temperature. The resulting mixture was heated at 100° C. for additional 6 h. Upon cooling to room temperature, the mixture was concentrated under reduced pressure, diluted with water (200 mL) and extracted with ethyl acetate (2×200 mL). The combined organic layers were washed with brine (2×100 mL), and dried over anhydrous sodium sulfate. After filtration, the filtrate was concentrated under reduced pressure; the residue was purified by silica gel column chromatography, and eluted with 20% ethyl acetate in petroleum ether to provide the desired product as a white solid (4.9 g, 35%). LCMS calculated for C₁₀H₁₃ClFN₂O₂ (M+H)⁺ m/z=247.1; found 247.0; ¹H NMR (400 MHz, CDCl₃) δ 8.13-8.04 (m, 2H), 7.00 (s, 1H), 1.54 (s, 9H).

### Step 2: Tert-butyl(3-fluoro-2-vinylpyridin-4-yl) carbamate

The mixture of tert-butyl(2-chloro-3-fluoropyridin-4-yl) carbamate (4.4 g, 17.8 mmol), 2-ethenyl-4,4,5,5-tetramethyl-1,3,2-dioxaborolane (3.3 g, 21.4 mmol), cesium carbonate (17.44 g, 53.51 mmol) and chloro(2-dicyclohexylphosphino-2′,4′,6′-triisoporpyl-1,1′-biphenyl)[2-(2′-amino-1,1′-biphenyl)]palladium(II) (702 mg, 0.89 mmol) in dioxane (40 mL) and water (8 mL) was heated at 80° C. for 2 h under nitrogen atmosphere. Upon cooling to room temperature, the resulting mixture was concentrated under reduced pressure. The residue was purified by silica gel column chromatography, and eluted with 20% ethyl acetate in petroleum ether to provide the desired product as a white solid (3.3 g, 77%). LCMS calculated for C₁₂H₁₆FN₂O₂ (M+H)⁺ m/z=239.1; found 239.1. ¹H NMR (300 MHz, CDCl₃) δ 8.34 (d, J=5.4 Hz, 1H), 8.08 (dd, J=5.4, 5.4 Hz, 1H), 7.09-7.00 (m, 2H), 6.50 (dd, J=17.4, 1.8 Hz, 1H), 5.68 (dd, J=11.1, 1.8 Hz, 1H), 1.55 (s, 9H).

### Step 3: Tert-butyl(3-fluoro-2-formylpyridin-4-yl) carbamate

The mixture of tert-butyl(3-fluoro-2-vinylpyridin-4-yl) carbamate (3.3 g, 13.85 mmol), 2,6-lutidine (2.97 g, 27.70 mmol) and potassium osmate (VI) dihydrate (1.02 g, 2.77 mmol) in dioxane (100 mL) and water (30 mL) was stirred for 30 min at room temperature, followed by the addition of sodium periodate (11.85 g, 55.40 mmol) in portions at room temperature. After stirring for additional 3 h, the mixture was diluted with water (200 mL) and extracted with ethyl acetate (2×200 mL). The combined organic layers were washed with brine (200 mL), and dried over anhydrous sodium sulfate. After filtration, the filtrate was concentrated under reduced pressure. The residue was purified by silica gel column chromatography, and eluted with 20% ethyl acetate in petroleum ether to provide the desired product as a white solid (1.58 g, 47%). LCMS calculated for C₁₁H₁₄FN₂O₃ (M+H)⁺ m/z=241.1; found 241.1; ¹H NMR (400 MHz, CDCl₃) δ 10.15 (s, 1H), 8.46 (d, J=5.6 Hz, 1H), 8.37 (dd, J=5.6, 5.6 Hz, 1H), 7.07 (s, 1H), 1.56 (s, 9H).

### Step 4: Tert-butyl(3-fluoro-2-(hydroxymethyl)pyridin-4-yl) carbamate

The mixture of tert-butyl(3-fluoro-2-formylpyridin-4-yl) carbamate (1.58 g, 6.58 mmol) was treated with sodium borohydride (373 mg, 9.9 mmol) in methanol (15 mL) in portions at 0° C. After stirring at room temperature for 1 h, the reaction was quenched with water (5 mL) at 0° C. The resulting mixture was concentrated under reduced pressure. The residue was purified by silica gel column chromatography, and eluted with 15% methanol in dichloromethane to provide the desired product as a white solid (1 g, 63%). LCMS calculated for C₁₁H₁₆FN₂O₃ (M+H)⁺ m/z=243.1; found 243.3; ¹H NMR (400 MHz, CDCl₃) δ 8.25 (d, J=5.6 Hz, 1H), 8.08 (dd, J=5.6, 5.6 Hz, 1H), 6.98 (s, 1H), 4.79 (d, J=2.0 Hz, 2H), 1.55 (s, 9H).

### Step 5: (4-Amino-3-fluoropyridin-2-yl)methanol

The mixture of tert-butyl(3-fluoro-2-(hydroxymethyl)pyridin-4-yl) carbamate (1 g, 4.13 mmol) in dichloromethane (10 mL) was treated with 2,2,2-trifluoroacetic acid (4 mL) at room temperature. After stirring for 16 h, the mixture was concentrated under vacuum and diluted with dichloromethane (50 mL), neutralized with sodium carbonate, and extracted with dichloromethane (3×50 mL). The combined organic layers were dried over anhydrous sodium sulfate. After filtration, the filtrate was concentrated under reduced pressure. The residue was purified by silica gel column chromatography, and eluted with 50% ethyl acetate in petroleum ether to provide the desired product as a white solid (420 mg, 72%). LCMS calculated for C₆H₈FN₂O (M+H)⁺ m/z=143.1; found 143.0; ¹H NMR (400 MHz, CD₃OD) δ 7.83 (d, J=5.6 Hz, 1H), 6.73-6.70 (m, 1H), 4.64 (d, J=2.4 Hz, 2H).

### Step 6: (4-Amino-5-bromo-3-fluoropyridin-2-yl)methanol

The mixture of (4-amino-3-fluoropyridin-2-yl)methanol (350 mg, 2.46 mmol) in acetonitrile (4 mL) was treated with 1-bromopyrrolidine-2,5-dione (438 mg, 2.46 mmol) at room temperature. After stirring for 16 h, the mixture was concentrated under vacuum and the residue was purified by silica gel column chromatography, and eluted with 50% ethyl acetate in petroleum ether to provide the desired product as a white solid (370 mg, 68%). LCMS calculated for C₆H₇BrFN₂O (M+H)⁺ m/z=221.0; found 220.9; ¹H NMR (400 MHz, CD₃OD) δ 8.10 (s, 1H), 4.63 (d, J=2.8 Hz, 2H).

### Step 7: 4-Fluoro-3-(hydroxymethyl)-5,7,8,9-tetrahydro-6H-cyclopenta[c][1,6]naphthyridin-6-one

The mixture of methyl 2-(4,4,5,5-tetramethyl-1,3,2-dioxaborolan-2-yl)cyclopent-1-ene-1-carboxylate (200 mg, 0.79 mmol), (4-amino-5-bromo-3-fluoropyridin-2-yl)methanol (175 mg, 0.79 mmol), 1,1′-bis(diphenylphosphino)ferrocene-palladium(II) dichloride dichloromethane complex (63 mg, 0.08 mmol) and sodium carbonate (168 mg, 1.59 mmol) in dioxane (5 mL) and added water (1 mL) was stirred for 16 h at 80° C. under nitrogen atmosphere. Upon cooling to room temperature, the mixture was concentrated under reduced pressure. The residue was purified by silica gel column chromatography, and eluted with 90% ethyl acetate in dichloromethane (0˜90%) to provide the desired product as a yellow solid (150 mg, 81%). LCMS calculated for C₁₂H₁₂FN₂O₂ (M+H)⁺ m/z=235.1; found 235.0.

### Step 8: 5-(4-((4-Fluoro-6-oxo-6,7,8,9-tetrahydro-5H-cyclopenta[c][1,6]naphthyridin-3-yl)methyl)piperazin-1-yl)-N,6-dimethylpicolinamide (I-25)

The mixture of 4-fluoro-3-(hydroxymethyl)-5,7,8,9-tetrahydro-6H-cyclopenta[c][1,6]naphthyridin-6-one (50 mg, 0.21 mmol) was combined with hydrogen bromide (33 wt. % solution in glacial acid, 1 mL) at room temperature. The reaction was then heated at 80° C. under nitrogen atmosphere for 2 h. Upon cooling to room temperature, the reaction was concentrated under reduced pressure. The residue was taken in 1-methylpyrrolidin-2-one (0.8 mL) followed by addition of N,6-dimethyl-5-(piperazin-1-yl)picolinamide hydrochloride (58 mg, 0.21 mmol), N-ethyl-N-isopropylpropan-2-amine (552 mg, 4.2 mmol). The resulting mixture was stirred at room temperature for 16 h; and then purified by prep-HPLC (column: SunFire Prep C18 OBD column 30*150 mm 5 um; mobile phase A: water (0.05% trifluoroacetic acid), mobile phase B: acetonitrile; flow rate: 60 mL/min); eluted fractions were collected and lyophilized to provide the TFA salt of the desired product as a white solid (25.8 mg). LCMS calculated for C₂₄H₂₈FN₆O₂ (M+H)⁺ m/z=451.2; found 451.1; ¹H NMR (400 MHz, DMSO-d₆) δ 12.38 (s, 1H), 8.71 (s, 1H), 8.47-8.43 (m, 1H), 7.84 (d, J=8.1 Hz, 1H), 7.55 (d, J=8.1 Hz, 1H), 4.73 (s, 2H), 3.54-3.51 (m, 4H), 3.23-3.19 (m, 6H), 2.83-2.79 (m, 5H), 2.52 (s, 3H), 2.20-2.13 (m, 2H).

### Example 26: 5-(4-((4-Fluoro-6-oxo-6,7,8,9-tetrahydro-5H-cyclopenta[c][1,6]naphthyridin-3-yl)methyl)piperazin-1-yl)-N-methylpicolinamide (I-26)

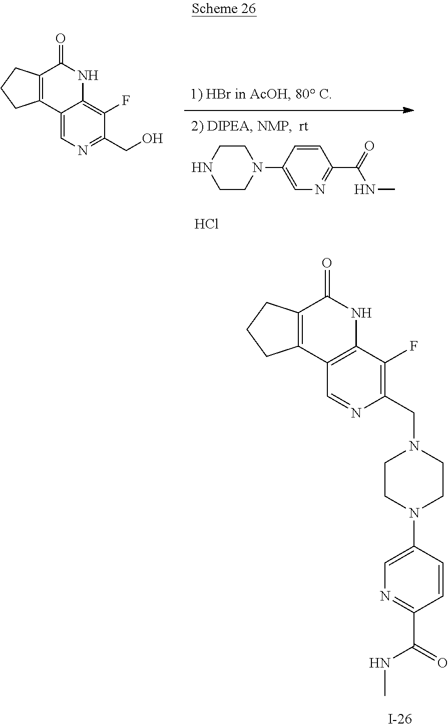

The mixture of 4-fluoro-3-(hydroxymethyl)-5,7,8,9-tetrahydro-6H-cyclopenta[c][1,6]naphthyridin-6-one (30 mg, 0.13 mmol) was combined with hydrogen bromide (33 wt. % solution in glacial acid, 1 mL) at room temperature. The reaction was then heated at 80° C. under nitrogen atmosphere for 2 h. Upon cooling to room temperature, the reaction was concentrated under reduced pressure. The residue was taken in 1-methylpyrrolidin-2-one (1 mL) followed by addition of N-methyl-5-(piperazin-1-yl)picolinamide dihydrochloride (37 mg, 0.13 mmol), N-ethyl-N-isopropylpropan-2-amine (335 mg, 2.6 mmol). The resulting mixture was stirred at room temperature for 16 h, and then purified by prep-HPLC (column: Xselect CSH C₁₈ OBD Column 30*150 mm 5 um; mobile phase A: water (0.05% trifluoroacetic acid), mobile phase B: acetonitrile; flow rate: 60 mL/min; gradient: 5% B to 40% B in 7 min); eluted fractions were collected and lyophilized to provide the TFA salt of the desired product as a white solid (2.2 mg). LCMS calculated for C₂₃H₂₆FN₆O₂ (M+H)⁺ m/z=437.2; found 437.0; ¹H NMR (400 MHz, CD₃OD) δ 8.73 (s, 1H), 8.37 (s, 1H), 7.97 (d, J=8.8 Hz, 1H), 7.50 (d, J=8.4 Hz, 1H), 4.75 (s, 2H), 3.75-3.69 (m, 4H), 3.66-3.61 (m, 4H), 3.29-3.25 (m, 2H), 2.95-2.91 (m, 5H), 2.32-2.24 (m, 2H).

### Example 27: 6-Fluoro-5-(4-((4-fluoro-6-oxo-6,7,8,9-tetrahydro-5H-cyclopenta[c][1,6]naphthyridin-3-yl)methyl)piperazin-1-yl)-N-methylpicolinamide (I-27)

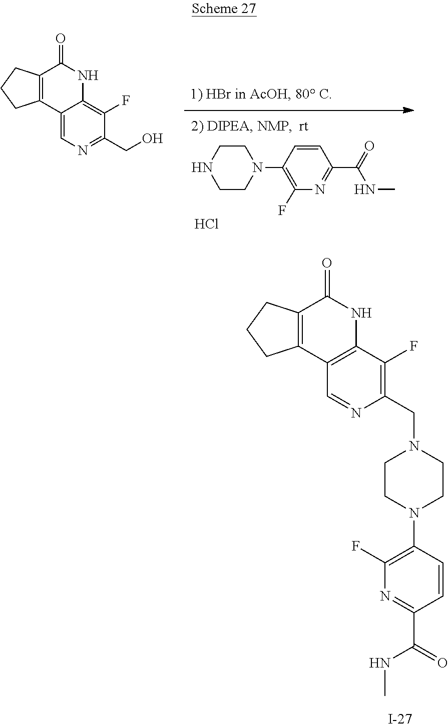

The mixture of 4-fluoro-3-(hydroxymethyl)-5,7,8,9-tetrahydro-6H-cyclopenta[c][1,6]naphthyridin-6-one (30 mg, 0.13 mmol) was combined with hydrogen bromide (33 wt. % solution in glacial acid, 1 mL) at room temperature; the reaction was then heated at 80° C. under nitrogen atmosphere for 2 h. Upon cooling to room temperature, the reaction was concentrated under reduced pressure. The residue was taken in 1-methylpyrrolidin-2-one (1 mL) followed by addition of 6-fluoro-N-methyl-5-(piperazin-1-yl)picolinamide hydrochloride (35 mg, 0.13 mmol) and N-ethyl-N-isopropylpropan-2-amine (335 mg, 2.6 mmol). The resulting mixture was stirred at room temperature for 16 h; and then purified by prep-HPLC (column: Xselect CSH C18 OBD Column 30*150 mm 5 um; mobile phase A: water (0.05% trifluoroacetic acid), mobile phase B: acetonitrile; flow rate: 60 mL/min; gradient: 10% B to 45% B in 7 min); eluted fractions were collected and lyophilized to provide the TFA salt of the desired product as a white solid (4.1 mg). LCMS calculated for C₂₃H₂₅F₂N₆O₂ (M+H)⁺ m/z=455.2; found 455.0; ¹H NMR (300 MHz, CD₃OD) δ 8.75 (s, 1H), 7.99-7.96 (m, 1H), 7.69-7.63 (m, 1H), 4.73 (s, 2H), 3.64-3.56 (m, 8H), 3.31-3.27 (m, 2H), 2.98-2.94 (m, 5H), 2.36-2.28 (m, 2H).

### Example 28: 5-(4-((4-Fluoro-6-oxo-6,7,8,9-tetrahydro-5H-cyclopenta[c][1,6]naphthyridin-3-yl)methyl)piperazin-1-yl)-N-methyl-6-(trifluoromethyl)picolinamide (I-28)

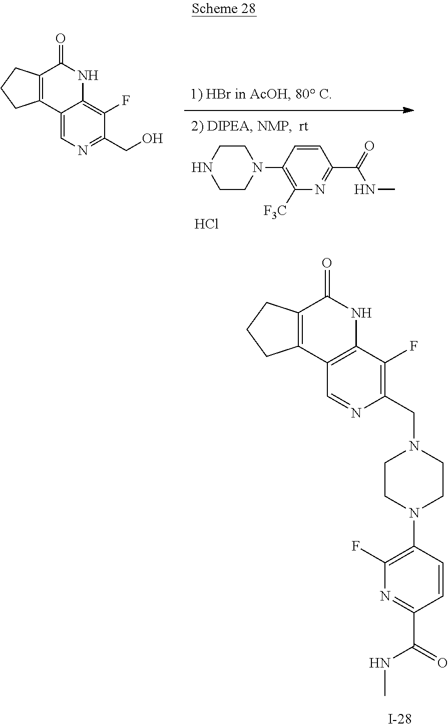

The mixture of 4-fluoro-3-(hydroxymethyl)-5,7,8,9-tetrahydro-6H-cyclopenta[c][1,6]naphthyridin-6-one (30 mg, 0.13 mmol) was combined with hydrogen bromide (33 wt. % solution in glacial acid, 1 mL) at room temperature. The reaction was then heated at 80° C. under nitrogen atmosphere for 2 h. Upon cooling to room temperature, the reaction was concentrated under reduced pressure. The residue was taken in 1-methylpyrrolidin-2-one (1 mL) followed by addition of N-methyl-5-(piperazin-1-yl)-6-(trifluoromethyl)picolinamide hydrochloride (41 mg, 0.13 mmol), and N-ethyl-N-isopropylpropan-2-amine (335 mg, 2.6 mmol). The resulting mixture was stirred at room temperature for 16 h; and then purified by prep-HPLC (column: Sunfire prep C₁₈ column, 30*150 mm, 5 um; mobile phase A: water (0.05% trifluoroacetic acid), mobile phase B: acetonitrile; flow rate: 60 mL/min; gradient: 11% B to 30% B in 7 min); eluted fractions were collected and lyophilized to provide the TFA salt of the desired product as a white solid (18.3 mg). LCMS calculated for C₂₄H₂₅F₄N₆O₂ (M+H)⁺ m/z=505.2; found 505.3; ¹H NMR (400 MHz, CD₃OD) δ 8.73 (s, 1H), 8.32 (d, J=8.4 Hz, 1H), 8.11 (d, J=8.8 Hz, 1H), 4.79 (s, 2H), 3.67-3.63 (m, 4H), 3.42-3.39 (m, 4H), 3.29-3.26 (m, 2H), 2.97 (s, 3H), 2.95-2.91 (m, 2H), 2.32-2.25 (m, 2H).

### Example 29: 5-(4-((3-Ethyl-2,4-dioxo-1,2,3,4-tetrahydroquinazolin-7-yl)methyl)piperazin-1-yl)-6-fluoro-N-methylpicolinamide (I-29)

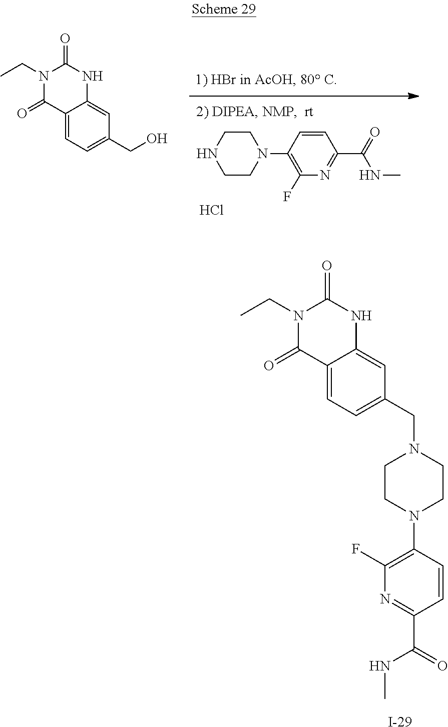

The mixture of 3-ethyl-7-(hydroxymethyl) quinazoline-2,4(1H,3H)-dione (Example 1, step 2, 20 mg, 0.09 mmol) was combined with hydrogen bromide (33 wt. % solution in glacial acid, 1 mL) at room temperature. The reaction was then heated at 80° C. under nitrogen atmosphere for 2 h. Upon cooling to room temperature, the reaction was concentrated under reduced pressure. The residue was taken in 1-methylpyrrolidin-2-one (0.8 mL) followed by addition of 6-fluoro-N-methyl-5-(piperazin-1-yl)picolinamide hydrochloride (27 mg, 0.1 mmol) and N-ethyl-N-isopropylpropan-2-amine (234 mg, 1.8 mmol). The resulting mixture was stirred at room temperature for 16 h, and then purified by prep-HPLC (column: Xselect CSH F-Phenyl OBD column, 19*250 mm, 5 um; mobile phase A: water (0.05% trifluoroacetic acid), mobile phase B: acetonitrile; flow rate: 25 mL/min; gradient: 29% B to 52% B over 9 min); eluted fractions were collected and lyophilized to provide the TFA salt of the desired product as a white solid (24.7 mg). LCMS calculated for C₂₂H₂₆FN₆O₃ (M+H)⁺ m/z=441.2; found 441.3; ¹H NMR (400 MHz, DMSO-d₆) δ 11.67 (s, 1H), 10.08 (s, 1H), 8.45-8.41 (m, 1H), 8.04-8.02 (m, 1H), 7.88 (d, J=7.6 Hz, 1H), 7.69-7.64 (m, 1H), 7.32-7.27 (m, 2H), 4.47 (s, 2H), 4.95 (q, J=7.2 Hz, 2H), 3.45-3.16 (m, 8H), 2.77 (d, J=4.8 Hz, 3H), 1.16 (t, J=7.2 Hz, 3H).

### Example 30: N,6-dimethyl-5-(4-((6-oxo-6,7,8,9-tetrahydro-5H-cyclopenta[c][1,6]naphthyridin-3-yl)methyl)piperazin-1-yl)picolinamide (1-30)

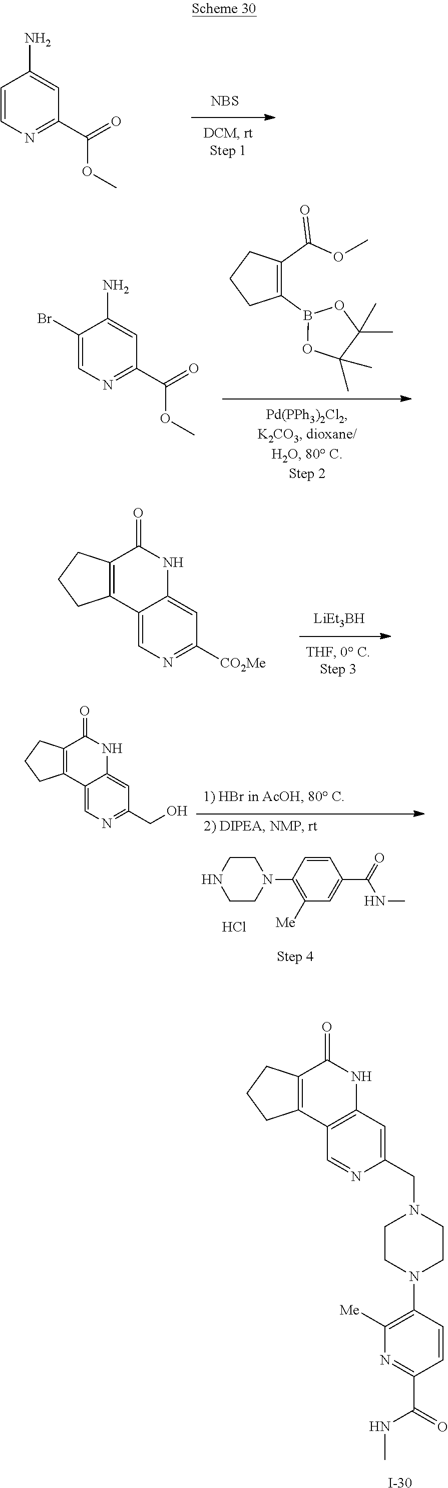

### Step 1: Methyl 4-amino-5-bromopicolinate

The mixture of methyl 4-aminopicolinate (5 g, 32.86 mmol) in 1,2-dichloroethane (80 mL) was treated with 1-bromopyrrolidine-2,5-dione (5 g, 32.86 mmol) in portions at 0° C. under nitrogen atmosphere, and stirred at room temperature for 16 h. The reaction mixture was then diluted with water (150 mL). The resulting mixture was extracted with dichloromethane (3×100 mL), and dried over sodium sulfate and filtered. The filtrate was concentrated under vacuum and the residue was purified by silica gel column chromatography, and eluted with 10% dichloromethane in methanol to provide the desired product as a white solid (3.2 g, 42%). LCMS calculated for C₇H₈BrN₂O₂ (M+H)⁺ m/z=231.0; found 231.1; ¹H NMR (300 MHz, CDCl₃) δ 8.33 (s, 1H), 7.42 (s, 1H), 6.64 (s, 2H), 3.82 (s, 3H).

### Step 2: Methyl 6-oxo-6,7,8,9-tetrahydro-5H-cyclopenta[c][1,6]naphthyridine-3-carboxylate

The mixture of methyl 2-(4,4,5,5-tetramethyl-1,3,2-dioxaborolan-2-yl)cyclopent-1-ene-1-carboxylate (500 mg, 1.98 mmol), methyl 4-amino-5-bromopicolinate (458 mg, 1.98 mmol), [1,1′-bis(diphenylphosphino)ferrocene]dichloropalladium(II) (161 mg, 0.20 mmol) and sodium carbonate (420 mg, 3.97 mmol) in dioxane (10 mL)) and water (2 mL) was stirred at 80° C. for 16 h under nitrogen atmosphere. Upon cooling to room temperature, the mixture was concentrated under vacuum and the residue was purified by reverse-phase flash chromatography (column: C18 silica gel; mobile phase: acetonitrile in water: elution: 5% to 50% gradient over 30 min; detector: UV 254 nm). The fractions were collected, combined and lyophilized to provide the desired product as a white solid (250 mg, 51%). LCMS calculated for C₁₃H₁₃N₂O₃ (M+H)⁺ m/z=245.1; found 245.0; ¹H NMR (400 MHz, CDCl₃) δ 10.59 (s, 1H), 8.85 (s, 1H), 8.05 (s, 1H), 4.05 (s, 3H), 3.28-3.24 (m, 2H), 3.05-3.01 (m, 2H), 2.32-2.29 (m, 2H).

### Step 3:3-(Hydroxymethyl)-5,7,8,9-tetrahydro-6H-cyclopenta[c][1,6]naphthyridin-6-one

The mixture of methyl 6-oxo-6,7,8,9-tetrahydro-5H-cyclopenta[c][1,6]naphthyridine-3-carboxylate (100 mg, 0.41 mmol) in anhydrous tetrahydrofuran (3 mL) was treated with lithium triethylhydroborate (1 M in tetrahydrofuran, 1.64 mL, 1.64 mmol) dropwise at 0° C. under nitrogen atmosphere. The resulting mixture was stirred at the same temperature for 1 h, which was then quenched with saturated aqueous solution of ammonium chloride, and extracted with dichloromethane (2×50 mL). The combined organics were dried over anhydrous sodium sulfate, filtered, and concentrated under reduced pressure. The residue was purified by silica gel column chromatography, and eluted with 10% dichloromethane in methanol to provide the desired product as a yellow solid (77 mg, 86%). LCMS calculated for C₁₂H₁₃N₂O₂ (M+H)⁺ m/z=217.1; found 217.0.

### Step 4: N,6-dimethyl-5-(4-((6-oxo-6,7,8,9-tetrahydro-5H-cyclopenta[c][1,6]naphthyridin-3-yl)methyl)piperazin-1-yl)picolinamide (I-30)

The mixture of 3-(hydroxymethyl)-5,7,8,9-tetrahydro-6H-cyclopenta[c][1,6]naphthyridin-6-one (35 mg, 0.16 mmol) was combined with hydrogen bromide (33 wt. % solution in glacial acid, 1 mL) at room temperature. The reaction was then heated at 80° C. under nitrogen atmosphere for 2 h. Upon cooling to room temperature, the reaction was concentrated under reduced pressure. The residue was taken in 1-methylpyrrolidin-2-one (1 mL) followed by addition of N,6-dimethyl-5-(piperazin-1-yl)picolinamide hydrochloride (44 mg, 0.16 mmol), N-ethyl-N-isopropylpropan-2-amine (418 mg, 3.2 mmol). The resulting mixture was stirred at room temperature for 16 h, and then purified by prep-HPLC (column: Xselect CSH C18 OBD Column 30*150 mm 5 um; mobile phase A: water (0.05% trifluoroacetic acid), mobile phase B: acetonitrile; flow rate: 60 mL/min; gradient: 13% B to 25% B in 8 min); eluted fractions were collected and lyophilized to provide the TFA salt of the desired product as a white solid (13.6 mg). LCMS calculated for C₂₄H₂₉N₆O₂ (M+H)⁺ m/z=433.2; found 433.1; ¹H NMR (400 MHz, DMSO-d₆) δ 12.20 (s, 1H), 10.34 (s, 1H), 8.86 (s, 1H), 8.46-8.43 (m, 1H), 7.84 (d, J=8.0 Hz, 1), 7.55 (d, J=8.0 Hz, 1H), 7.39 (s, 1H), 4.62 (s, 2H), 3.45-3.42 (m, 4H), 3.24-3.13 (m, 6H), 2.82-2.77 (m, 5H), 2.52 (s, 3H), 1.28-1.25 (m, 2H).

### Example 31: 5-(4-((3-Ethyl-2,4-dioxo-1,2,3,4-tetrahydroquinazolin-7-yl)methyl)piperazin-1-yl)-N-methyl-6-(trifluoromethyl)picolinamide (I-31)

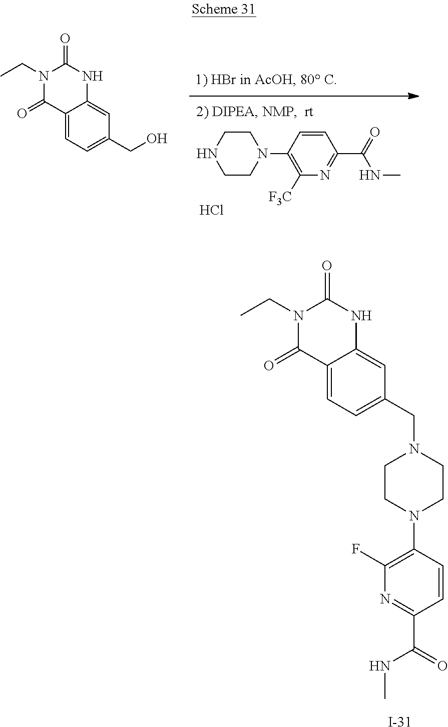

The mixture of 3-ethyl-7-(hydroxymethyl) quinazoline-2,4(1H,3H)-dione (Example 1, step 2, 20 mg, 0.09 mmol) was combined with hydrogen bromide (33 wt. % solution in glacial acid, 1 mL) at room temperature. The reaction was then heated at 80° C. under nitrogen atmosphere for 2 h. Upon cooling to room temperature, the reaction was concentrated under reduced pressure. The residue was taken in 1-methylpyrrolidin-2-one (0.8 mL) followed by addition of N-methyl-5-(piperazin-1-yl)-6-(trifluoromethyl)picolinamide hydrochloride (32 mg, 0.1 mmol) and N-ethyl-N-isopropylpropan-2-amine (234 mg, 1.8 mmol). The resulting mixture was stirred at room temperature for 16 h, and then purified by Prep-HPLC with the following conditions (column: Xselect CSH C₁₈ OBD Column 30*150 mm 5 um; mobile phase A: water (0.05% trifluoroacetate), mobile phase B: acetonitrile; flow rate: 60 mL/min; gradient: 10% B to 35% B over 7 min); eluted fractions were collected and lyophilized to give the TFA salt of the desired product as a white solid (14.7 mg). LCMS calculated for C₂₃H₂₆F₃N₆O₃ (M+H)⁺ m/z=491.2; found 491.3; ¹H NMR (400 MHz, CD₃OD) δ 8.31 (d, J=8.4 Hz, 1H), 8.16 (d, J=8.0 Hz, 1H), 8.07 (d, J=8.4 Hz, 1H), 7.39 (dd, J=8.4, 1.6 Hz, 1H), 7.33 (d, J=1.6 Hz, 1H), 4.48 (s, 2H), 4.08 (q, J=7.2 Hz, 2H), 3.48-3.43 (m, 4H), 3.34-3.31 (m, 4H), 2.97 (s, 3H), 1.25 (t, J=7.2 Hz, 3H).

### Example 32: 5-(4-((5-chloro-3-ethyl-2,4-dioxo-1,2,3,4-tetrahydroquinazolin-7-yl)methyl)piperazin-1-yl)-N,6-dimethylpicolinamide (I-32)

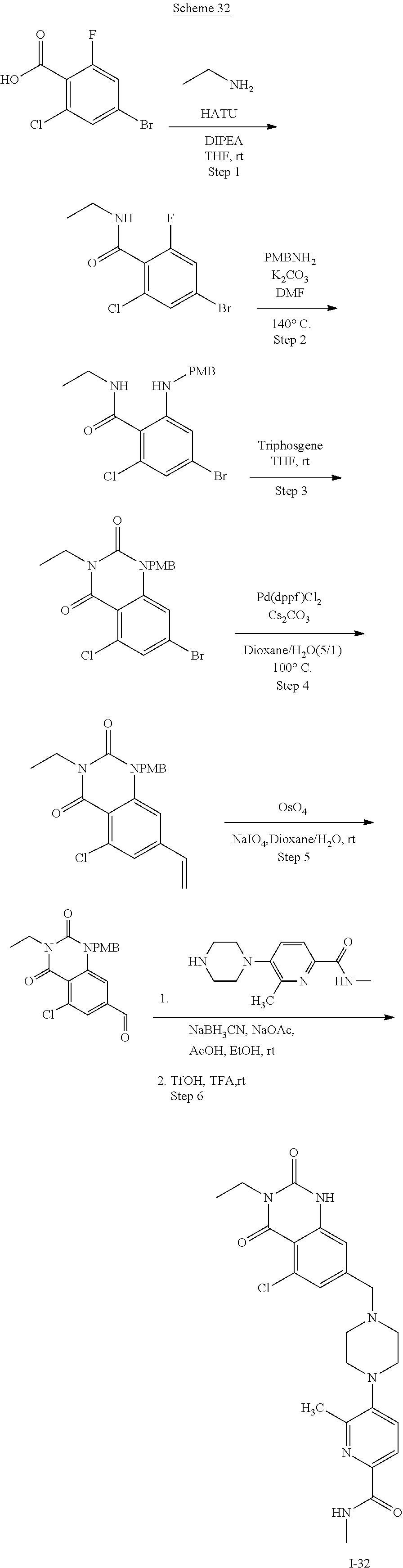

### Step 1: 4-bromo-2-chloro-N-ethyl-6-fluorobenzamide

To a mixture of 4-bromo-2-chloro-6-fluorobenzoic acid (2.0 g, 7.89 mmol) in tetrahydrofuran (34 mL) were added 1-[Bis(dimethylamino)methylene]-1H-1,2,3-triazolo[4,5-b]pyridinium 3-oxid hexafluorophosphate (3.6 g, 9.47 mmol), N-ethyl-N-isopropylpropan-2-amine (2.75 mL, 15.78 mmol) and 2 M solution of ethylamine in THF (5.92 mL, 11.84 mmol) at room temperature. The resulting mixture was stirred at room temperature for 16 h. Upon completion of reaction, the resulting mixture was concentrated under reduced pressure. The residue was purified by silica gel column chromatography, eluted with 20% ethyl acetate in hexanes to provide the desired product as a white solid (1.77 g, 80%). LCMS calculated for C₉H₉BrClFNO (M+H)⁺ m/z=280.0; found 279.9.

### Step 2: 4-bromo-2-chloro-N-ethyl-6-((4-methoxybenzyl)amino)benzamide

The mixture of 4-bromo-2-chloro-N-ethyl-6-fluorobenzamide (1.77 g, 4.17 mmol), potassium carbonate (2.62 g, 18.93 mmol), 4-Methoxybenzylamine (1.81 mL, 13.88 mmol) in dimethylformamide (40 mL) was stirred at 140° C. for 8 h. Upon cooling to room temperature, the mixture was diluted with ethyl acetate (80 mL) and washed with water (4×50 mL). The organic layer was dried with anhydrous sodium sulfate. After filtration, the filtrate was concentrated under reduced pressure. The residue was purified by silica gel column chromatography, eluted with 0 to 30% ethyl acetate in hexanes to provide the desired product as a white solid (1.37 g, 55%). LCMS calculated for C₁₇H₁₉BrClN₂O₂ (M+H)⁺ m/z=397.0; found 397.0.

### Step 3: 7-bromo-5-chloro-3-ethyl-1-(4-methoxybenzyl) quinazoline-2,4(1H,3H)-dione

To a solution of 4-bromo-2-chloro-N-ethyl-6-((4-methoxybenzyl)amino)benzamide (1.37 g, 3.44 mmol) in tetrahydrofuran (34 mL) was added N,N-Diisopropylethylamine (6.0 mL, 34.45 mmol) and triphosgene (1.52 g, 5.17 mmol) at 0° C. The resulting mixture was stirred at room temperature for 16 h, and then quenched with saturated solution of ammonium chloride in water (20 mL). The organic layers were washed with brine (20 mL) and dried with anhydrous sodium sulfate. After filtration, the filtrate was concentrated under reduced pressure, and the residue was purified by silica gel column chromatography, eluted with 0% to 30% ethyl acetate in hexane to provide the desired product as a light yellow solid (0.45 g, 32%). LCMS calculated for C₁₈H₁₇BrClN₂O₃ (M+H)⁺ m/z=423.0; found 423.0.

### Step 4: 5-chloro-3-ethyl-1-(4-methoxybenzyl)-7-vinylquinazoline-2,4(1H,3H)-dione

The mixture of 7-bromo-5-chloro-3-ethyl-1-(4-methoxybenzyl) quinazoline-2,4(1H,3H)-dione (370 mg, 0.87 mmol), 2-ethenyl-4,4,5,5-tetramethyl-1,3,2-dioxaborolane (200.59 μL, 1.14 mmol), cesium carbonate (853.6 mg, 2.62 mmol) and 1,1′-bis(diphenylphosphino)ferrocene-palladium(II) dichloride dichloromethane complex (142.6 mg, 0.17 mmol) in 1,4-dioxane (8.7 mL) and water (1.5 mL) was stirred at 80° C. for 4 h under nitrogen atmosphere. Upon cooling to room temperature, the mixture was diluted with water (15 mL), and then extracted with ethyl acetate (3×10 mL). The combined organic layers were dried with anhydrous sodium sulfate. After filtration, the filtrate was concentrated under reduced pressure. The residue was purified by silica gel column chromatography, eluted with 0 to 20% ethyl acetate in hexane to provide the desired product as a yellow oil (68.3 mg, crude). LCMS calculated for C₂₀H₂₀ClN₂O₃ (M+H)⁺ m/z=371.1; found 371.1.

### Step 5: 5-chloro-3-ethyl-1-(4-methoxybenzyl)-2,4-dioxo-1,2,3,4-tetrahydroquinazoline-7-carbaldehyde

To a solution of 3-ethyl-5-fluoro-7-vinylquinazoline-2,4(1H,3H)-dione (68.3 mg, 0.18 mmol) and sodium metaperiodate (157.6 mg, 0.74 mmol) in 1,4-dioxane (1.8 mL) and water (0.3 mL) was added 4% solution of osium tetraoxide in water (233.8 μL, 0.04 mmol). The resulting mixture was stirred at room temperature for 5 h, and then diluted with ethyl acetate (10 mL). The organic layers were washed with water (2×5 mL) and dried with anhydrous sodium sulfate. After filtration, the filtrate was concentrated under reduced pressure, and the residue was purified by silica gel column chromatography, eluted with 50% ethyl acetate in hexane to provide the desired product as a white solid (12 mg, 18%). LCMS calculated for C₁₉H₁₈ClN₂O₄ (M+H)⁺ m/z=373.1; found 373.1.

### Step 6: 5-(4-((5-chloro-3-ethyl-2,4-dioxo-1,2,3,4-tetrahydroquinazolin-7-yl)methyl)piperazin-1-yl)-N,6-dimethylpicolinamide (I-32)

To a solution of 5-chloro-3-ethyl-1-(4-methoxybenzyl)-2,4-dioxo-1,2,3,4-tetrahydroquinazoline-7-carbaldehyde (12 mg, 0.03 mmol) in ethanol (0.4 mL) was added sodium cyanoborohydride (5.6 mg, mmol), sodium acetate (6.8 mg), acetic acid (5.6 μL) at room temperature. The resulting mixture was stirred at room temperature for 16 h; and then solvent was removed under reduced pressure, and the crude was dissolved in a mixture of triflic acid (0.2 mL) and trifluoroacetic acid (2.1 mL) at room temperature. The resulting mixture was stirred at room temperature for 2 h. The mixture was purified by prep-HPLC (column: SunFire Prep C18 OBD column 30*150 mm 5 um; mobile phase A: water (0.05% trifluoroacetic acid), mobile phase B: acetonitrile; flow rate: 60 mL/min); eluted fractions were collected and lyophilized to provide the TFA salt of the desired product as a white solid (2.0 mg). LCMS calculated for C₂₃H₂₈ClN₆O₃ (M+H)⁺ m/z=471.1; found 471.2.

### Example 33: 5-(4-((3-Ethyl-5-fluoro-2,4-dioxo-1,2,3,4-tetrahydroquinazolin-7-yl)methyl)piperazin-1-yl)-6-fluoro-N-methylpicolinamide (I-33)

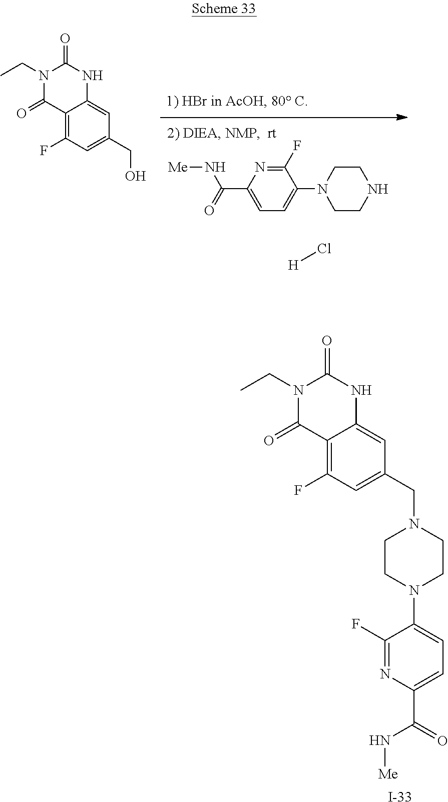

The mixture of 3-ethyl-5-fluoro-7-(hydroxymethyl) quinazoline-2,4(1H,3H)-dione (Example 22, step 4, 100 mg, 0.42 mmol) was combined with hydrogen bromide (33 wt. % solution in glacial acid, 2 mL) at room temperature; the reaction was then heated at 80° C. under nitrogen atmosphere for 2 h. Upon cooling to room temperature, the reaction was concentrated under reduced pressure. The residue was taken in 1-methylpyrrolidin-2-one (2 mL), followed by addition of 6-fluoro-N-methyl-5-(piperazin-1-yl)picolinamide hydrochloride (116 mg, 0.42 mmol) and N-ethyl-N-isopropyl-2-methylpropan-1-amine (542 mg, 4.2 mmol). The resulting mixture was stirred at room temperature for 16 h, and then purified by prep-HPLC (column: Xselect CSH C18 OBD Column 30*150 mm 5 um; mobile phase A: water (0.05% trifluoroacetic acid), mobile phase B: acetonitrile; flow rate: 60 mL/min; gradient: 16% B to 30% B over 7 min); eluted fractions were collected and lyophilized to provide the TFA salt of the desired product as a white solid (93 mg). LCMS calculated for C₂₂H₂₅F₂N₆O₃ (M+H)⁺ m/z=459.2; found 459.1; ¹H NMR (300 MHz, CD₃OD) δ 7.96 (d, J=7.8 Hz, 1H), 7.70-7.58 (m, 1H), 7.18-7.06 (m, 2H), 4.47 (s, 2H), 4.07 (q, J=6.9 Hz, 2H), 3.58-3.46 (s, 8H), 2.93 (s, 3H), 1.26 (t, J=6.9 Hz, 3H).

### Example 34: 5-(4-((3-Ethyl-6-fluoro-1-methyl-4-oxo-1,3,4,5-tetrahydropyrazolo[3,4,5-de]quinazolin-7-yl)methyl)piperazin-1-yl)-N,6-dimethylpicolinamide (I-34)

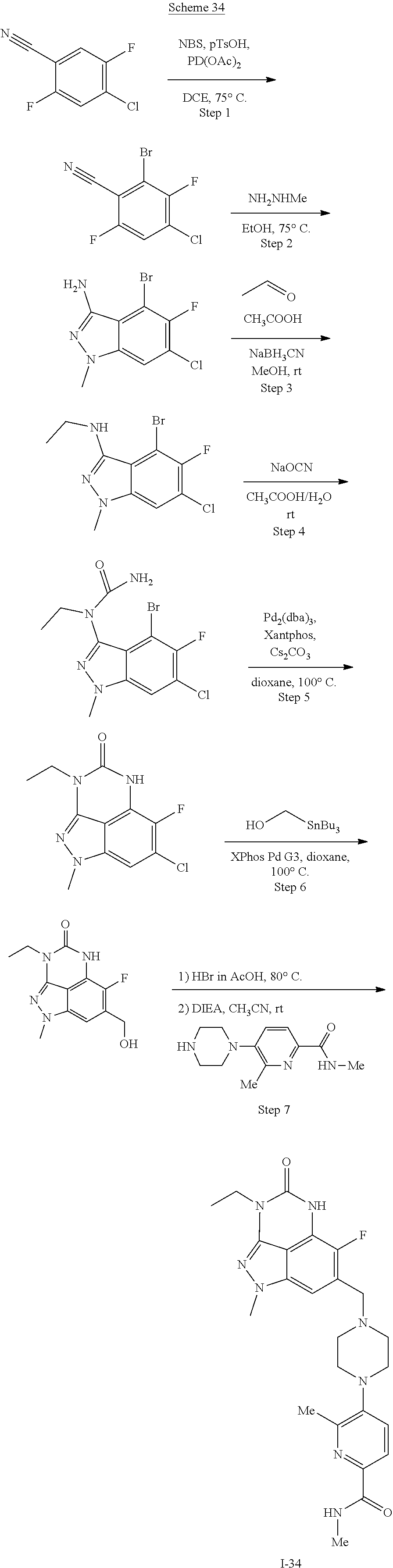

### Step 1:2-bromo-4-chloro-3,6-difluorobenzonitrile

A mixture of 4-chloro-2,5-difluorobenzonitrile (6 g, 34.57 mmol), N-bromosuccinimide (6.77 g, 38.03 mmol), palladium(II) acetate (0.78 g, 3.46 mmol) and p-toluenesulfonic acid (3.29 g, 17.29 mmol) in 1,2-dichloroethane (60 mL) was stirred at 70° C. for 12 h under nitrogen atmosphere. Upon cooling to room temperature, the reaction mixture was concentrated under reduced pressure. The resulting residue was purified by silica gel column chromatography, eluted with 50% ethyl acetate in petroleum ether to provide the desired product as a white solid (2.2 g, 25%). ¹H NMR (300 MHz, CDCl₃) δ 7.36 (dd, J=7.8, 5.4 Hz, 1H).

### Step 2: 4-bromo-6-chloro-5-fluoro-1-methyl-1H-indazol-3-amine

A mixture of 2-bromo-4-chloro-3,6-difluorobenzonitrile (2 g, 7.92 mmol) and methyl hydrazine (40% in water, 844 mg, 7.33 mmol) in ethanol (8 mL) was stirred at 80° C. for 16 h. Upon cooling to room temperature, the reaction mixture was diluted with water (200 mL) and extracted with ethyl acetate (3×300 mL). The combined organic layers were washed with brine (2×100 mL) and dried over anhydrous sodium sulfate. After filtration, the filtrate was concentrated under reduced pressure. The resulting residue was purified by silica gel column chromatography, eluted with 30% ethyl acetate in petroleum ether to provide the desired product as a white solid (1.8 g, 82%). LCMS calculated for C₈H₇BrClFN₃ (M+H)⁺ m/z=277.9; found 280.0; ¹H NMR (400 MHz, CDCl₃) δ 7.19 (d, J=2.8 Hz, 1H), 3.79 (s, 3H).

### Step 3: 4-bromo-6-chloro-N-ethyl-5-fluoro-1-methyl-1H-indazol-3-amine

A mixture of 4-bromo-6-chloro-5-fluoro-1-methylindazol-3-amine (1 g, 3.59 mmol) and acetaldehyde (127 mg, 2.87 mmol) in methanol (3 mL) was treated with acetic acid (103 mg, 1.72 mmol) dropwise for 1 h; followed by the addition of sodium cyanoborohydride (108 mg, 1.72 mmol) at 0° C. The resulting mixture was stirred for additional 1 h; and then concentrated under reduced pressure. The resulting residue was purified by silica gel column chromatography, eluted with 30% ethyl acetate in petroleum ether to provide the desired product as a white solid (300 mg, 27%). LCMS calculated for C₁₀H₁₁BrClFN₃ (M+H)⁺ m/z=306.0; found 306.0; ¹H NMR (400 MHz, CDCl₃) δ 7.15 (d, J=5.6 Hz, 1H), 3.79 (s, 3H), 3.42 (q, J=7.2 Hz, 2H), 1.33 (t, J=7.2 Hz, 3H).

### Step 4: 1-(4-bromo-6-chloro-5-fluoro-1-methyl-1H-indazol-3-yl)-1-ethylurea

A mixture of 4-bromo-6-chloro-N-ethyl-5-fluoro-1-methylindazol-3-amine (300 mg, 0.98 mmol) in acetic acid (6 mL) and water (2 mL) was treated with sodium cyanate (191 mg, 2.94 mmol) for 3 h at room temperature; and then diluted with water (10 mL), extracted with ethyl acetate (3×60 mL). The combined organic layers were washed with saturated sodium bicarbonate (2×60 mL), dried over anhydrous sodium sulfate. After filtration, the filtrate was concentrated under reduced pressure. The resulting residue was purified by silica gel column chromatography, eluted with 30% ethyl acetate in petroleum ether to provide the desired product as a white solid (200 mg, 58%). LCMS calculated for C₁₁H₁₂BrClFN₄O (M+H)⁺ m/z=349.0; found 349.0; ¹H NMR (400 MHz, CDCl₃) δ 7.46 (d, J=5.6 Hz, 1H), 4.38 (s, 2H), 4.05 (s, 3H), 3.33 (q, J=7.2 Hz, 2H), 1.18 (t, J=7.2 Hz, 3H).

### Step 5: 7-chloro-3-ethyl-6-fluoro-1-methyl-1,5-dihydropyrazolo[3,4,5-de]quinazolin-4 (3H)-one

A mixture of 1-(4-bromo-6-chloro-5-fluoro-1-methylindazol-3-yl)-1-ethylurea (180 mg, 0.52 mmol), cesium carbonate (335 mg, 1.03 mmol), tris(dibenzylideneacetone) dipalladium (236 mg, 0.26 mmol) and 4,5-bis(diphenylphosphino)-9,9-dimethylxanthene (298 mg, 0.52 mmol) in dioxane (5 mL) was stirred at 100° C. for 16 h under nitrogen atmosphere. Upon cooling to room temperature, the reaction mixture was concentrated under vacuum. The resulting residue was purified by silica gel column chromatography, eluted with 50% ethyl acetate in petroleum ether to provide the desired product as a yellow solid (120 mg, 87%). LCMS calculated for C₁₁H₁₁ClFN₄O (M+H)⁺ m/z=269.1; found 269.1; ¹H NMR (400 MHz, CDCl₃) δ 7.41 (s, 1H), 6.66 (d, J=3.6 Hz, 1H), 3.97 (q, J=7.2 Hz, 2H), 3.81 (s, 3H), 1.36 (t, J=7.2 Hz, 3H).

### Step 6:3-ethyl-6-fluoro-7-(hydroxymethyl)-1-methyl-1,5-dihydropyrazolo[3,4,5-de]quinazolin-4 (3H)-one

A mixture of 7-chloro-3-ethyl-6-fluoro-1-methyl-1,5-dihydropyrazolo[3,4,5-de]quinazolin-4 (3H)-one (110 mg, 0.41 mmol), methanesulfonato (2-dicyclohexylphosphino-2′,4′,6′-tri-i-propyl-1,1′-biphenyl)(2′-amino-1,1′-biphenyl-2-yl)palladium(II) (69 mg, 0.082 mmol) and (tributylstannyl)methanol (263 mg, 0.82 mmol) in 1,4-dioxane (5 mL) was stirred at 100° C. for 2 h under nitrogen atmosphere. Upon cooling to room temperature, the reaction mixture was concentrated under reduced pressure. The resulting residue was purified by silica gel column chromatography, eluted with 10% methanol in dichloromethane to provide the desired product as a white solid (80 mg, 74%). LCMS calculated for C₁₂H₁₄FN₄O₂ (M+H)⁺ m/z=265.1; found 265.2;

### Step 7: 5-(4-((3-ethyl-6-fluoro-1-methyl-4-oxo-1,3,4,5-tetrahydropyrazolo[3,4,5-de]quinazolin-7-yl)methyl)piperazin-1-yl)-N,6-dimethylpicolinamide (I-34)

A mixture of 3-ethyl-6-fluoro-7-(hydroxymethyl)-1-methyl-1,5-dihydropyrazolo[3,4,5-de]quinazolin-4 (3H)-one (80 mg, 0.3 mmol) was combined with hydrogen bromide (33 wt. % solution in glacial acid, 3 mL) at room temperature. The reaction mixture was then heated at 80° C. under nitrogen atmosphere for 2 h. Upon cooling to room temperature, the reaction mixture was concentrated under reduced pressure. The residue was taken in acetonitrile (10 mL), followed by addition of N,6-dimethyl-5-(piperazin-1-yl)pyridine-2-carboxamide (81.6 mg, 0.35 mmol) and N,N-Diisopropylethylamine (783 mg, 6.1 mmol). The resulting mixture was stirred at room temperature for additional 16 h, and then concentrated under reduced pressure. The residue was purified by silica gel column chromatography, eluted with 10% methanol in dichloromethane. After concentration, the residue was purified by reversed-phase flash chromatography with the following conditions: (column, C18 silica gel; mobile phase, acetonitrile in water (0.1% trifluoroacetate), 5% to 50% gradient in 30 min; detector, UV 254 nm). The fractions were collected, combined and lyophilized to provide the TFA salt of the desired product as a white solid (84 mg). LCMS calculated for C₂₄H₃₀FN₈O₂ (M+H)⁺ m/z=481.2; found 481.2; ¹H NMR (400 MHz, CD₃OD) δ 7.90 (d, J=8.4 Hz, 1H), 7.57 (d, J=8.4 Hz, 1H), 6.92 (d, J=3.6 Hz, 1H), 4.56 (s, 2H), 3.93 (q, J=7.2 Hz, 2H), 3.87 (s, 3H), 3.76-3.59 (m, 2H), 3.58-3.37 (m, 4H), 3.25-3.05 (m, 2H), 2.94 (s, 3H), 2.60 (s, 3H), 1.32 (t, J=7.2 Hz, 3H).

### Example 35: 5-(4-((3-ethyl-9-fluoro-2-oxo-2,3-dihydro-1H-pyrimido[4,5,6-de]quinazolin-8-yl)methyl)piperazin-1-yl)-N,6-dimethylpicolinamide (I-35)

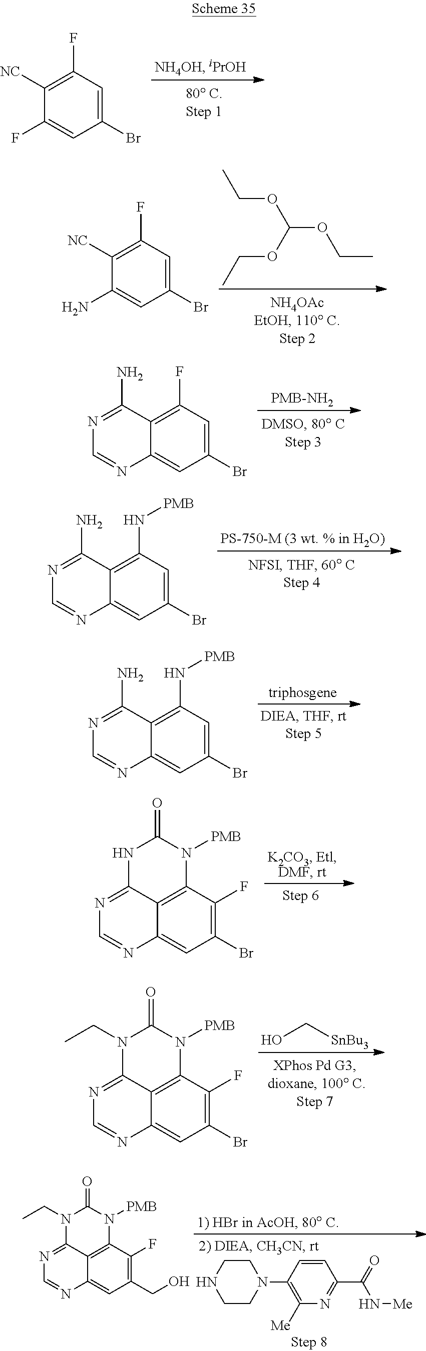

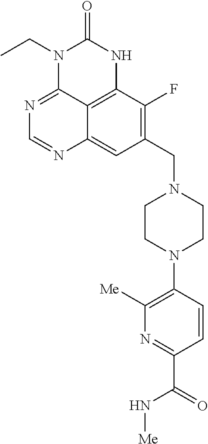 [SMILES: CCN1C(=O)Nc2c(F)c(CN3CCN(c4ccc(C(=O)NC)nc4C)CC3)cc3ncnc1c23]

### Step 1:2-amino-4-bromo-6-fluorobenzonitrile

A mixture of 4-bromo-2,6-difluorobenzonitrile (10 g, 45.87 mmol) and ammonium hydroxide (16.08 g, 458.7 mmol) in propan-2-ol (25 mL) was stirred at 80° C. for 16 h. Upon cooling to room temperature, the reaction mixture was diluted with water (500 mL). The precipitated solids were collected by filtration and washed with water (2×100 mL). The residue was purified by silica gel column chromatography, eluted with 20% ethyl acetate in petroleum ether to provide the desired product as a white solid (5.8 g, 59%). LCMS calculated for C₇H₃BrFN₂ (M−H)⁻ m/z=213.0; found 212.9.

### Step 2: 7-bromo-5-fluoroquinazolin-4-amine

A mixture of 2-amino-4-bromo-6-fluorobenzonitrile (3.9 g, 18.14 mmol), ammonium acetate (21 g, 272.1 mmol) and triethyl orthoformate (40.32 g, 272.1 mmol) in ethanol (40 mL) was stirred at 110° C. for 16 h. Upon cooling to room temperature, the reaction mixture was concentrated under reduced pressure. The residue was purified by silica gel column chromatography, eluted with 50% ethyl acetate in petroleum ether to provide the desired product as a yellow solid (3.3 g, 75%). LCMS calculated for C₈H₆BrFN₃ (M+H)⁺ m/z=242.0; found 242.0.

### Step 3: 7-bromo-N⁵-(4-methoxybenzyl) quinazoline-4,5-diamine

A mixture of 7-bromo-5-fluoroquinazolin-4-amine (1 g, 4.13 mmol) and (4-methoxyphenyl) methanamine (763.2 mg, 6.2 mmol) in dimethyl sulfoxide (10 mL) was stirred for 5 h at 80° C. Upon cooling to room temperature, the reaction mixture was diluted with water (100 mL), extracted with ethyl acetate (3×100 mL) and dried over anhydrous sodium sulfate. After filtration, the filtrate was concentrated under reduced pressure. The residue was purified by silica gel column chromatography, eluted with 80% ethyl acetate in dichloromethane to afford a yellow solid (1 g, 67%). LCMS calculated for C₁₆H₁₆BrN₄O (M+H)⁺ m/z=359.1; found 359.0.

### Step 4: 7-bromo-6-fluoro-N⁵-(4-methoxybenzyl) quinazoline-4,5-diamine

A mixture of 7-bromo-N⁵-(4-methoxybenzyl) quinazoline-4,5-diamine (1 g, 2.78 mmol) in PS-750-M (3% in water, 12 mL) was stirred at room temperature for 2 min under nitrogen atmosphere, followed by the addition of N-fluorobenzenesulfonimide (2.19 g, 6.96 mmol) in THF (12 mL) in portions over 5 min at room temperature. The resulting mixture was stirred at 60° C. for additional 16 h. Upon cooling to room temperature, the reaction mixture was concentrated under reduced pressure. The residue was purified by silica gel column chromatography, eluted with 7% methanol in dichloromethane to afford the desired product (450 mg, 43%) as a yellow solid. LCMS calculated for C₁₆H₁₅BrFN₄O (M+H)⁺ m/z=377.0; found 377.0; ¹H NMR (300 MHz, CDCl₃) δ 8.49 (s, 1H), 7.91 (d, J=6.6 Hz, 1H), 7.59 (s, 2H), 7.26-7.19 (m, 2H), 6.96-6.88 (m, 2H), 4.10 (d, J=6.3 Hz, 2H), 3.85 (s, 3H). ¹⁹F NMR (377 MHz, CDCl₃) δ −130.20.

### Step 5: 8-bromo-9-fluoro-1-(4-methoxybenzyl)-1H-pyrimido[4,5,6-de]quinazolin-2 (3H)-one

A mixture of 7-bromo-6-fluoro-N⁵-[(4-methoxyphenyl)methyl]quinazoline-4,5-diamine (710 mg, 1.88 mmol), triphosgene (1117 mg, 3.76 mmol) and N,N-diisopropylethylamine (486.5 mg, 3.76 mmol) in tetrahydrofuran (10 mL) was stirred for 1 h at room temperature under nitrogen atmosphere; and then quenched with sat. ammonium chloride (aq.) at 0° C. The precipitated solids were collected by filtration and washed with water (3×100 mL). The residue was purified by trituration with ethyl acetate (100 mL). The resulting product was a yellow solid (400 mg, 53%) which was used in the next step without further purification. LCMS calculated for C₁₇H₁₃BrFN₄O₂ (M+H)⁺ m/z=403.0; found 403.1.

### Step 6: 8-bromo-3-ethyl-9-fluoro-1-(4-methoxybenzyl)-1H-pyrimido[4,5,6-de]quinazolin-2(3H)-one

A mixture of 8-bromo-9-fluoro-1-(4-methoxybenzyl)-1H-pyrimido[4,5,6-de]quinazolin-2(3H)-one (170 mg, 0.42 mmol) and potassium carbonate (116.5 mg, 0.84 mmol) in N,N-dimethylformamide (3 mL) was treated with ethyl iodide (131.5 mg, 0.84 mmol) at 0° C. The resulting mixture was stirred at room temperature for 16 h; and then diluted with water (30 mL), extracted with ethyl acetate (3×50 mL). The combined organic layers were washed with brine (2×50 mL), dried over anhydrous sodium sulfate. After filtration, the filtrate was concentrated under reduced pressure. The resulting residue was purified by silica gel column chromatography, eluted with 10% methanol in dichloromethane to afford a yellow solid (145 mg, 80%). LCMS calculated for C₁₉H₁₇BrFN₄O₂ (M+H)⁺ m/z=431.1; found 431.0.

### Step 7:3-ethyl-9-fluoro-8-(hydroxymethyl)-1-(4-methoxybenzyl)-1H-pyrimido[4,5,6-de]quinazolin-2(3H)-one

A mixture of 8-bromo-3-ethyl-9-fluoro-1-(4-methoxybenzyl)-1H-pyrimido[4,5,6-de]quinazolin-2(3H)-one (126 mg, 0.29 mmol), XPhos Pd G3 (50 mg, 0.06 mmol) and (tributylstannyl)methanol (141 mg, 0.44 mmol) in dioxane (2 mL) was stirred for 2 h at 100° C. under nitrogen atmosphere. Upon cooling to room temperature, the mixture was concentrated under reduced pressure. The residue was purified by silica gel column chromatography, eluted with 10% methanol in dichloromethane to provide the desired product (80 mg, 72%) as a white solid. LCMS calculated for C₂₀H₂₀FN₄O₃ (M+H)⁺ m/z=383.2; found 383.0.

### Step 8: 5-(4-((3-ethyl-9-fluoro-2-oxo-2,3-dihydro-1H-pyrimido[4,5,6-de]quinazolin-8-yl)methyl)piperazin-1-yl)-N,6-dimethylpicolinamide (1-35)

A mixture of 3-ethyl-9-fluoro-8-(hydroxymethyl)-1-(4-methoxybenzyl)-1H-pyrimido[4,5,6-de]quinazolin-2(3H)-one (80 mg, 0.21 mmol) was combined with hydrogen bromide (33 wt. % solution in glacial acid, 3 mL) at room temperature. The reaction was then heated at 80° C. under nitrogen atmosphere for 2 h. Upon cooling to room temperature, the reaction was concentrated under reduced pressure. The residue was taken in acetonitrile (10 mL), followed by addition of N,6-dimethyl-5-(piperazin-1-yl)pyridine-2-carboxamide (63 mg, 0.27 mmol) and N,N-diisopropylethylamine (290 mg, 2.25 mmol). The resulting mixture was stirred at room temperature for additional 16 h, and then concentrated under reduced pressure. The residue was purified by silica gel column chromatography, eluted with 10% methanol in dichloromethane. After concentration, the residue was purified by reversed-phase flash chromatography with the following conditions: (column, C18 silica gel; mobile phase, acetonitrile in water (0.1% trifluoroacetic acid), 5% to 50% gradient in 30 min; detector, UV 254 nm). The fractions were collected, combined and lyophilized to provide the TFA salt of the desired product as a light-yellow solid (86 mg). LCMS calculated for C₂₄H₂₈FN₈O₂ (M+H)⁺ m/z=479.2; found 479.3. ¹H NMR (400 MHz, CD₃OD) δ 8.78 (s, 1H), 7.90 (d, J=8.4 Hz, 1H), 7.61-7.53 (m, 2H), 4.71 (s, 2H), 4.31 (q, J=7.2 Hz, 2H), 3.66-3.56 (m, 4H), 3.34-3.24 (m, 4H), 2.94 (s, 3H), 2.60 (s, 3H), 1.33 (t, J=7.2 Hz, 3H).

### Example 36: 5-(4-((3-Ethyl-5-fluoro-2,4-dioxo-1,2,3,4-tetrahydroquinazolin-7-yl)methyl)piperazin-1-yl)-N,6-bis(methyl-d3)picolinamide (I-36)

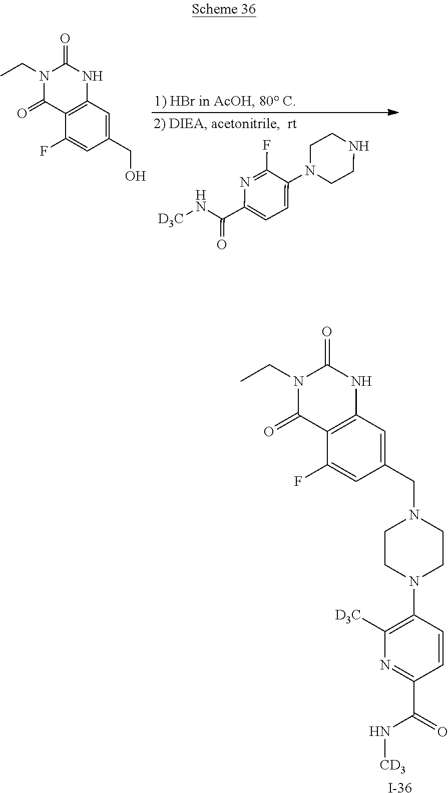

To 3-ethyl-5-fluoro-7-(hydroxymethyl)-1H-quinazoline-2,4-dione (Example 22, Step 4:106 mg, 0.45 mmol) was added hydrogen bromide (33 wt. % solution in glacial acid, 1.5 mL) at room temperature. The reaction mixture was then heated at 80° C. under nitrogen atmosphere for 2 h. Upon cooling to room temperature, the mixture was concentrated under reduced pressure. The residue was taken in acetonitrile (5 mL), followed by addition of N,6-bis(methyl-d₃)-5-(piperazin-1-yl)picolinamide (107 mg, 0.45 mmol) and N-ethyl-N-isopropylpropan-2-amine (0.8 mL). The resulting mixture was stirred at room temperature for additional 16 h, and then concentrated under reduced pressure. The resulting residue was purified by Prep-HPLC with the following conditions (column: YMC-Actus Triart C18, 30*150 mm, 5 um; mobile phase A: water (0.1% trifluoroacetic acid), mobile phase B: acetonitrile; Flow rate: 50 mL/min; Gradient: 12% B to 35% B over 7 min, detector, UV 254/220 nm). Eluted fractions were collected and lyophilized to give the TFA salt of the desired product as a white solid (66 mg). LCMS calculated for C₂₃H₂₂D₆FN₆O₃ (M+H)⁺ m/z=461.3; found 461.1; ¹H NMR (400 MHz, CD₃OD) δ 7.91 (d, J=8.4 Hz, 1H), 7.58 (d, J=8.4 Hz, 1H), 7.17-7.09 (m, 2H), 4.49 (s, 2H), 4.05 (q, J=7.2 Hz, 2H), 3.62-3.46 (m, 4H), 3.30-3.17 (m, 4H), 1.24 (t, J=7.2 Hz, 3H).

### Example 37. 5-(4-((5-(Difluoromethyl)-3-ethyl-2,4-dioxo-1,2,3,4-tetrahydroquinazolin-7-yl)methyl)piperazin-1-yl)-N,6-dimethylpicolinamide (I-37)

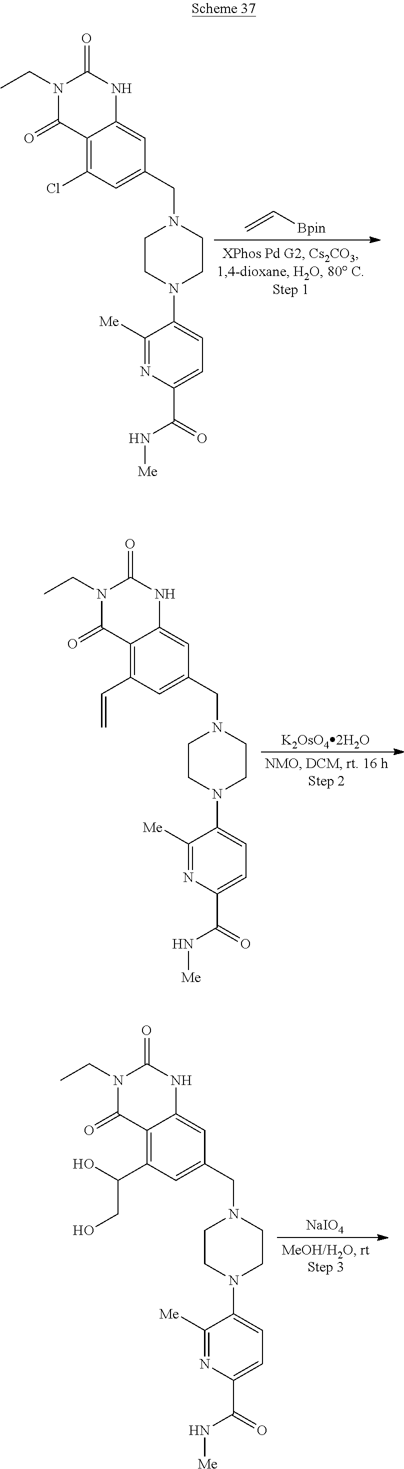

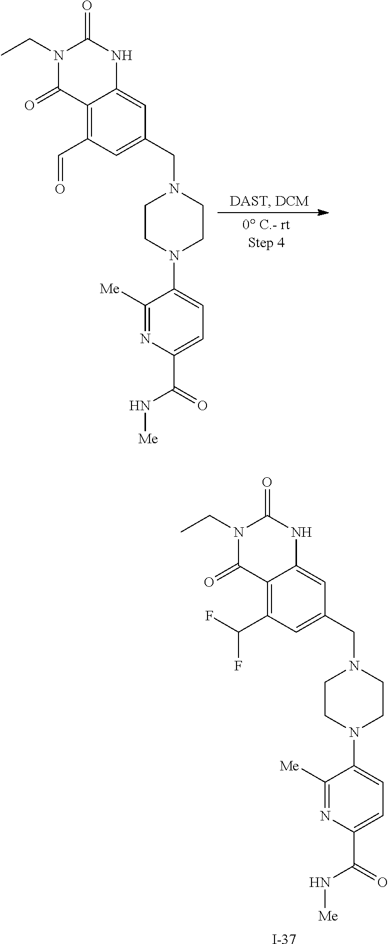

### Step 1: 5-(4-((3-Ethyl-2,4-dioxo-5-vinyl-1,2,3,4-tetrahydroquinazolin-7-yl)methyl)piperazin-1-yl)-N,6-dimethylpicolinamide

A mixture of 5-{4-[(5-chloro-3-ethyl-2,4-dioxo-1H-quinazolin-7-yl)methyl]piperazin-1-yl}-N,6-dimethylpyridine-2-carboxamide (Example 32:440 mg, 0.93 mmol), 2-ethenyl-4,4,5,5-tetramethyl-1,3,2-dioxaborolane (216 mg, 1.4 mmol), chloro(2-dicyclohexylphosphino-2′,4′,6′-tri-i-propyl-1,1′-biphenyl)(2′-amino-1,1′-biphenyl-2-yl)palladium(II) (73 mg, 0.09 mmol), and cesium carbonate (609 mg, 1.87 mmol) in dioxane (5 mL) and water (1 mL) was stirred at 80° C. for 2 h under nitrogen atmosphere. Upon cooling to room temperature, the reaction mixture was concentrated under reduced pressure. The resulting residue was purified by silica gel column chromatography, eluted with 6% methanol in dichloromethane to provide the desired product as a light-yellow solid (350 mg, 81%). LCMS calculated for C₂₅H₃₁N₆O₃ (M+H)⁺ m/z=463.2; found 463.2.

### Step 2: 5-(4-((5-(1,2-Dihydroxyethyl)-3-ethyl-2,4-dioxo-1,2,3,4-tetrahydroquinazolin-7-yl)methyl)piperazin-1-yl)-N,6-dimethylpicolinamide

A mixture of 5-(4-((3-ethyl-2,4-dioxo-5-vinyl-1,2,3,4-tetrahydroquinazolin-7-yl)methyl)piperazin-1-yl)-N,6-dimethylpicolinamide (150 mg, 0.32 mmol) and potassium osmate (VI) dihydrate (12 mg, 0.03 mmol) in dichloromethane (15 mL) and tert-butanol (1.5 mL) was treated with 4-methylmorpholine N-oxide (228 mg, 0.97 mmol) in portions at room temperature under nitrogen atmosphere. The reaction mixture was stirred for 16 h; then concentrated under reduced pressure. The resulting residue was purified by silica gel column chromatography, eluted with 10% methanol in dichloromethane to provide the desired product as a white solid (100 mg, 62%). LCMS calculated for C₂₅H₃₃N₆O₅ (M+H)⁺ m/z=497.2; found 497.3.

### Step 3: 5-(4-((3-Ethyl-5-formyl-2,4-dioxo-1,2,3,4-tetrahydroquinazolin-7-yl)methyl)piperazin-1-yl)-N,6-dimethylpicolinamide

A mixture of 5-(4-((5-(1,2-dihydroxyethyl)-3-ethyl-2,4-dioxo-1,2,3,4-tetrahydroquinazolin-7-yl)methyl)piperazin-1-yl)-N,6-dimethylpicolinamide (100 mg, 0.2 mmol) in methanol (2.5 mL) and water (2.5 mL) was treated with sodium periodate (64.6 mg, 0.3 mmol) in portions at room temperature. The reaction mixture was stirred for 30 min; then concentrated under reduced pressure. The resulting residue was purified by silica gel column chromatography, eluted with 10% methanol in dichloromethane to provide the desired product as a white solid (80 mg, 86%). LCMS calculated for C₂₄H₂₉N₆O₄ (M+H)⁺ m/z=465.2; found 465.3.

### Step 4: 5-(4-((5-(Difluoromethyl)-3-ethyl-2,4-dioxo-1,2,3,4-tetrahydroquinazolin-7-yl)methyl)piperazin-1-yl)-N,6-dimethylpicolinamide (I-37)

A mixture of 5-(4-((3-ethyl-5-formyl-2,4-dioxo-1,2,3,4-tetrahydroquinazolin-7-yl)methyl)piperazin-1-yl)-N,6-dimethylpicolinamide (130 mg, 0.28 mmol) in dichloromethane (5 mL, 78.6 mmol) was added diethylaminosulfur trifluoride (226 mg, 1.4 mmol) dropwise portions at 0° C. under nitrogen atmosphere. The reaction mixture was stirred for 24 h at room temperature under nitrogen atmosphere; then quenched with saturated ammonium chloride (1 mL) at 0° C., and concentrated under reduced pressure. The resulting residue was purified by silica gel column chromatography, eluted with 10% methanol in dichloromethane to provide the desired product. The product was re-purified via reversed-phase flash chromatography with the following conditions: column, C18 silica gel; mobile phase, acetonitrile in water (0.1% trifluoroacetic acid), 10% to 50% gradient in 25 min; detector, UV 254 nm. The fractions were combined and lyophilized to provide the TFA salt of the desired product as a white solid (85 mg). LCMS calculated for C₂₄H₂₉F₂N₆O₃ (M+H)⁺ m/z=487.2; found 487.2. ¹H NMR (400 MHz, CD₃OD) δ 8.07-7.87 (m, 2H), 7.78 (d, J=1.2 Hz, 1H), 7.57 (d, J=8.4 Hz, 1H), 7.49 (d, J=1.2 Hz, 1H), 4.58 (s, 2H), 4.07 (q, J=7.2 Hz, 2H), 3.61-3.45 (m, 4H), 3.35-3.17 (m, 4H), 2.94 (s, 3H), 2.60 (s, 3H), 1.25 (t, J=7.2 Hz, 3H).

### Example 38: 5-(4-((8-Fluoro-5-methoxy-3-methyl-2,4-dioxo-1,2,3,4-tetrahydroquinazolin-7-yl)methyl)piperazin-1-yl)-N,6-dimethylpicolinamide (I-38)

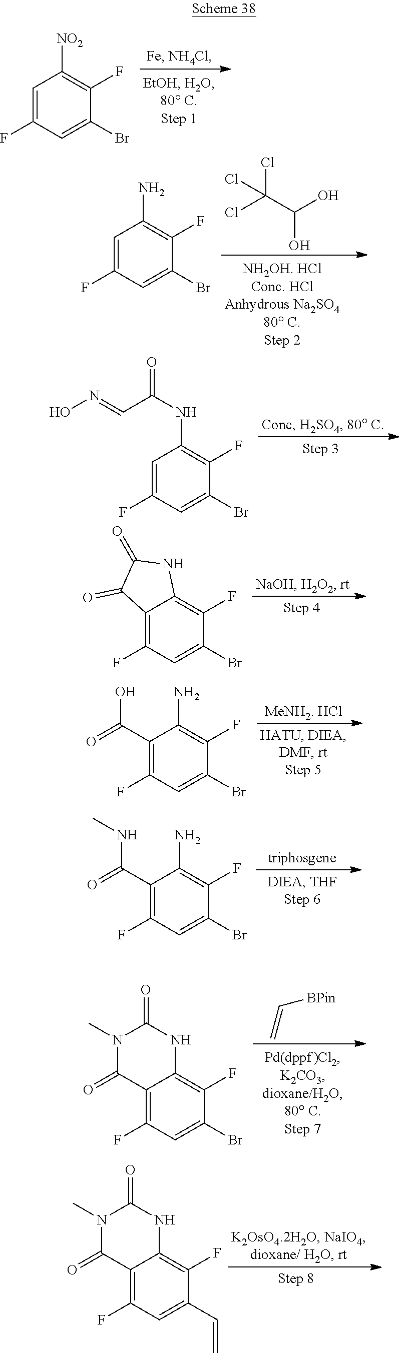

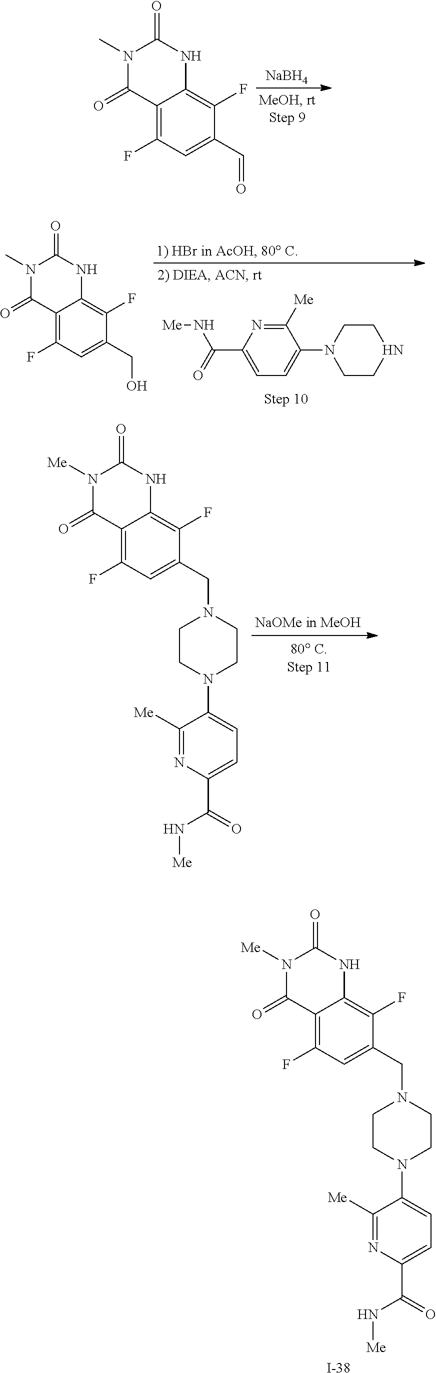

### Step 1:3-Bromo-2,5-difluoroaniline

A mixture of 1-bromo-2,5-difluoro-3-nitrobenzene (20 g, 84.0 mmol), iron (23.5 g, 420.2 mmol) and ammonium chloride (13.5 g, 252.1 mmol) in ethanol (180 mL) and water (20 mL) was stirred at 80° C. for 2 h. Upon cooling to room temperature, the reaction mixture was filtered, the filter cake was washed with ethanol (3×100 mL). The filtrate was concentrated under reduced pressure. The resulting residue was purified by silica gel column chromatography, eluted with 15% ethyl acetate in petroleum ether to provide the desired product as yellow solid (16.5 g, 94%). LCMS calculated for C₆H₅BrF₂N (M+H)⁺ m/z=208.0; found 207.9. ¹H NMR (300 MHz, CDCl₃) δ 6.67-6.61 (m, 1H), 6.49-6.42 (m, 1H).

### Step 2: (E)-N-(3-bromo-2,5-difluorophenyl)-2-(hydroxyimino) acetamide

To a mixture of 3-bromo-2,5-difluoroaniline (16.4 g, 78.8 mmol), chloral hydrate (19.6 g, 118.3 mmol), hydroxylamine hydrochloride (17.5 g, 252.3 mmol), sodium sulfate (67.2 g, 473.1 mmol) in water (180 mL) was added hydrochloric acid (6M, 4.1 mL). The reaction mixture was stirred at 80° C. for 5 h. Upon cooling to room temperature, the precipitate was collected by filtration, washed with water (3×100 mL), and dried under vacuum to provide the desired product as an off-white solid (20 g, 90%). LCMS calculated for C₈H₆BrF₂N₂O₂ (M+H)⁺ m/z=279.0; found 279.1.

### Step 3: 6-Bromo-4,7-difluoroindoline-2,3-dione

A mixture of (E)-N-(3-bromo-2,5-difluorophenyl)-2-(hydroxyimino) acetamide (10 g, 35.8 mmol) in concentrated sulfuric acid (100 mL) was stirred for 3 h at 80° C. Upon cooling to 0° C.; the mixture was neutralized to pH=7 with 20% sodium hydroxide aqueous solution, and then extracted with ethyl acetate (3×200 mL). The combined organic layers were dried over anhydrous sodium sulfate. After filtration, the filtrate was concentrated under reduced pressure. The resulting residue was purified by silica gel column chromatography, eluted with 50% ethyl acetate in petroleum ether to provide the desired product as a yellow solid (8.3 g, 88%). LCMS calculated for C₈H₃BrF₂NO₂ (M+H)⁺ m/z=261.9; found 261.8. ¹H NMR (300 MHz, CDCl₃) δ 7.09-7.05 (m, 1H).

### Step 4:2-Amino-4-bromo-3,6-difluorobenzoic acid

A mixture of 6-bromo-4,7-difluoroindoline-2,3-dione (5 g, 19.1 mmol) and sodium hydroxide (7.1 g, 177.5 mmol) in water (50 mL) was treated with hydrogen peroxide (30%, 3.38 g, 99.2 mmol) dropwise at 0° C. The reaction mixture was stirred at room temperature for 16 h; and then neutralized to pH 7 with conc. hydrochloric acid. The resulting mixture was concentrated under reduced pressure. The residue was purified by reversed-phase flash chromatography with the following conditions: column, C18 silica gel; mobile phase, acetonitrile in water (0.1% formic acid), 5% to 55% gradient in 20 min; detector, UV 254 nm. The fractions were collected, combined and lyophilized to provide the desired product as a yellow solid (2.2 g, 46%). LCMS calculated for C₇H₃BrF₂NO₂ (M−H)⁻ m/z=249.9; found 249.8.

### Step 5:2-Amino-4-bromo-3,6-difluoro-N-methylbenzamide

A mixture of 2-amino-4-bromo-3,6-difluorobenzoic acid (3 g, 11.9 mmol) and 2-(7-azabenzotriazol-1-yl)-N,N,N′,N′-tetramethyluronium hexafluorophosphate (5.43 g, 14.3 mmol) in N,N-dimethylformamide (60 mL) was stirred for 20 min at room temperature. Then methylamine hydrochloride (0.96 g, 14.3 mmol) was added, followed by N,N-diisopropylethylamine (4.61 g, 35.7 mmol). The reaction mixture was stirred for additional 16 h; then diluted with water (200 mL) and extracted with ethyl acetate (3×300 mL). The combined organic layers were washed with brine (2×500 mL), dried over anhydrous sodium sulfate. After filtration, the filtrate was concentrated under reduced pressure. The resulting residue was purified by silica gel column chromatography, eluted with 50% ethyl acetate in petroleum ether to provide the desired product as a white solid (2.1 g, 67%). LCMS calculated for C₈H₈BrF₂N₂O (M+H)⁺ m/z=265.0; found 265.0. ¹H NMR (400 MHz, CDCl₃) δ 6.66 (s, 1H), 6.53 (dd, J=12.0, 5.2 Hz, 1H), 6.26 (s, 2H), 2.97 (s, 3H).

### Step 6: 7-Bromo-5,8-difluoro-3-methylquinazoline-2,4(1H,3H)-dione

A mixture of 2-amino-4-bromo-3,6-difluoro-N-methylbenzamide (2 g, 7.5 mmol) and N,N-diisopropylethylamine (1.95 g, 15.1 mmol) in tetrahydrofuran (60 mL) was treated with triphosgene (2.24 g, 7.5 mmol) at 0° C. The reaction mixture was stirred for 1 h at room temperature; then concentrated under reduced pressure. The resulting residue was purified by silica gel column chromatography, eluted with 7% methanol in dichloromethane to provide the desired product as a white solid (2 g, 91%). LCMS calculated for C₉H₄BrF₂N₂O₂ (M−H)⁻ m/z=289.0; found 289.1. ¹H NMR (400 MHz, DMSO-d₆) δ 11.89 (s, 1H), 7.48 (dd, J=12.0, 5.2 Hz, 1H), 3.26 (s, 3H).

### Step 7:5,8-Difluoro-3-methyl-7-vinylquinazoline-2,4(1H,3H)-dione

A mixture of 7-bromo-5,8-difluoro-3-methylquinazoline-2,4(1H,3H)-dione (2.2 g, 7.5 mmol), 2-ethenyl-4,4,5,5-tetramethyl-1,3,2-dioxaborolane (1.75 g, 11.3 mmol), 1,1′-bis(diphenylphosphino)ferrocene-palladium(II) dichloride dichloromethane complex (0.62 g, 0.7 mmol) and potassium carbonate (2.1 g, 15.1 mmol) in dioxane (30 mL) and water (6 mL) was stirred for 4 h at 80° C. under nitrogen atmosphere. Upon cooling to room temperature, the reaction mixture was concentrated under reduced pressure. The resulting residue was purified by silica gel column chromatography, eluted with 60% ethyl acetate in dichloromethane to provide the desired product as a yellow solid (1.4 g, 78%). LCMS calculated for C₁₁H₉F₂N₂O₂ (M+H)⁺ m/z=239.1; found 239.2. ¹H NMR (400 MHz, DMSO-d₆) δ 11.63 (s, 1H), 7.30 (dd, J=12.0, 5.2 Hz, 1H), 6.87 (dd, J=17.6, 11.2 Hz, 1H), 6.18 (d, J=17.6 Hz, 1H), 5.68 (d, J=11.2 Hz, 1H), 3.21 (s, 3H).

### Step 8:5,8-Difluoro-3-methyl-2,4-dioxo-1,2,3,4-tetrahydroquinazoline-7-carbaldehyde

A mixture of 5,8-difluoro-3-methyl-7-vinylquinazoline-2,4(1H,3H)-dione (1.2 g, 5.0 mmol) and potassium osmate (VI) dihydrate (0.19 g, 0.5 mmol), sodium periodate (2.16 g, 10.1 mmol) in dioxane (30 mL) and water (6 mL) was stirred for 2 h at room temperature. The resulting mixture was concentrated under reduced pressure. The resulting residue was purified by silica gel column chromatography, eluted with 10% methanol in dichloromethane to provide the desired product as a white solid (0.9 g, 74%). LCMS calculated for C₁₀H₅F₂N₂O₃ (M−H)⁻ m/z=239.0; found 238.9. ¹H NMR (400 MHz, DMSO-d₆) δ 12.00 (s, 1H), 10.22 (s, 1H), 7.23 (dd, J=12.0, 5.2 Hz, 1H), 3.23 (s, 3H).

### Step 9:5,8-Difluoro-7-(hydroxymethyl)-3-methylquinazoline-2,4(1H,3H)-dione

A mixture of 5,8-difluoro-3-methyl-2,4-dioxo-1,2,3,4-tetrahydroquinazoline-7-carbaldehyde (850 mg, 3.54 mmol) in methanol (25 mL) was treated with sodium borohydride (268 mg, 7.1 mmol) portion-wise at 0° C. The reaction mixture was stirred at room temperature for 30 min; then concentrated under reduced pressure. The resulting residue was purified by silica gel column chromatography, eluted with 10% methanol in dichloromethane to provide the desired product as a white solid (500 mg, 56%). LCMS calculated for C₁₀H₉F₂N₂O₃ (M+H)⁺ m/z=243.1; found 243.0.

### Step 10: 5-(4-((5,8-Difluoro-3-methyl-2,4-dioxo-1,2,3,4-tetrahydroquinazolin-7-yl)methyl)piperazin-1-yl)-N,6-dimethylpicolinamide

A mixture of 5,8-difluoro-7-(hydroxymethyl)-3-methylquinazoline-2,4(1H,3H)-dione (120 mg, 0.5 mmol) and hydrogen bromide (33 wt. % solution in glacial acid, 2 mL) was stirred for 2 h at 80° C. Upon cooling to room temperature, the reaction mixture was concentrated under reduced pressure. To the residue was added acetonitrile (2 mL), followed by addition of N,6-dimethyl-5-(piperazin-1-yl)picolinamide (128 mg, 0.5 mmol) and N-ethyl-N-isopropylpropan-2-amine (512 mg, 4.0 mmol). The resulting mixture was stirred for additional 16 h; then concentrated under reduced pressure. The resulting residue was purified by silica gel column chromatography, eluted with 10% methanol in dichloromethane to provide the desired product as a white solid (190 mg, 84%). LCMS calculated for C₂₂H₂₅F₂N₆O₃ (M+H)⁺ m/z=459.2; found 459.0.

### Step 11: 5-(4-((8-fluoro-5-methoxy-3-methyl-2,4-dioxo-1,2,3,4-tetrahydroquinazolin-7-yl)methyl)piperazin-1-yl)-N,6-dimethylpicolinamide (I-38)

A mixture of 5-(4-((5,8-difluoro-3-methyl-2,4-dioxo-1,2,3,4-tetrahydroquinazolin-7-yl)methyl)piperazin-1-yl)-N,6-dimethylpicolinamide (120 mg, 0.26 mmol) and sodium methoxide (33 wt. % solution in methanol, 5 mL) was stirred for 2 h at 80° C. Upon cooling to room temperature, the reaction mixture was concentrated under reduced pressure. The resulting residue was purified by Prep-HPLC with the following conditions (Column: XBridge Prep Phenyl OBD Column, 19*150 mm, 5 um; mobile Phase A: water (0.05% trifluoroacetic acid), mobile phase B: acetonitrile; Flow rate: 25 mL/min; gradient: 5% B to 20% B over 8 min, detector: UV 254/220 nm). Eluted fractions were collected and lyophilized to give the TFA salt of the desired product as a white solid (63 mg). LCMS calculated for C₂₃H₂₈FN₆O₄ (M+H)⁺ m/z=471.2; found 471.2. ¹H NMR (300 MHz, CD₃OD) δ 7.90 (d, J=8.1 Hz, 1H), 7.58 (d, J=8.4 Hz, 1H), 6.95 (d, J=5.1 Hz, 1H), 4.59 (s, 2H), 3.97 (s, 3H), 3.67-3.56 (m, 4H), 3.36 (s, 3H), 3.31-3.21 (m, 4H), 2.94 (s, 3H), 2.60 (s, 3H).

### Example 39: 5-(4-((5-chloro-3-ethyl-8-fluoro-2,4-dioxo-1,2,3,4-tetrahydroquinazolin-7-yl)methyl)piperazin-1-yl)-N,6-dimethylpicolinamide (I-39)

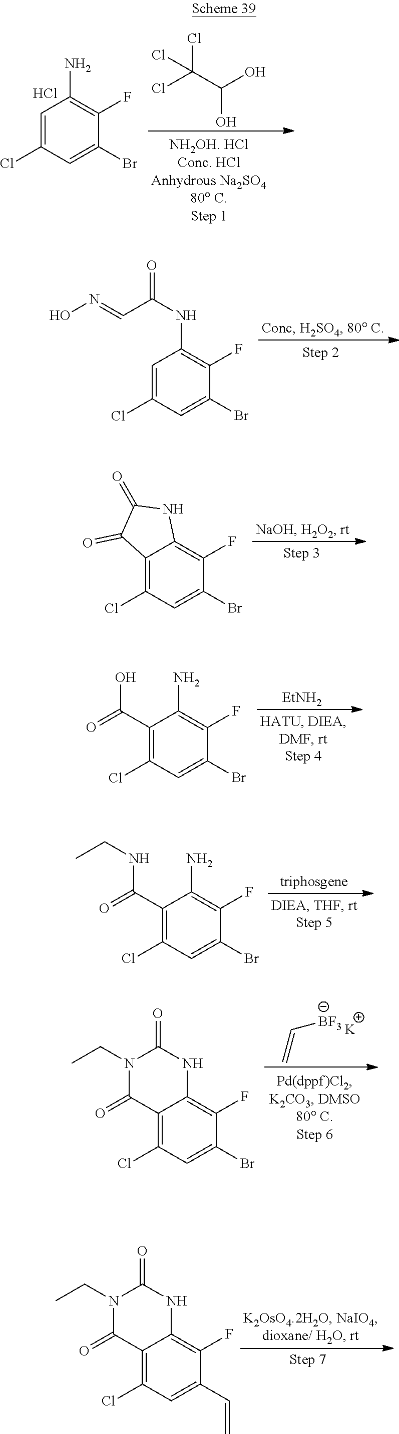

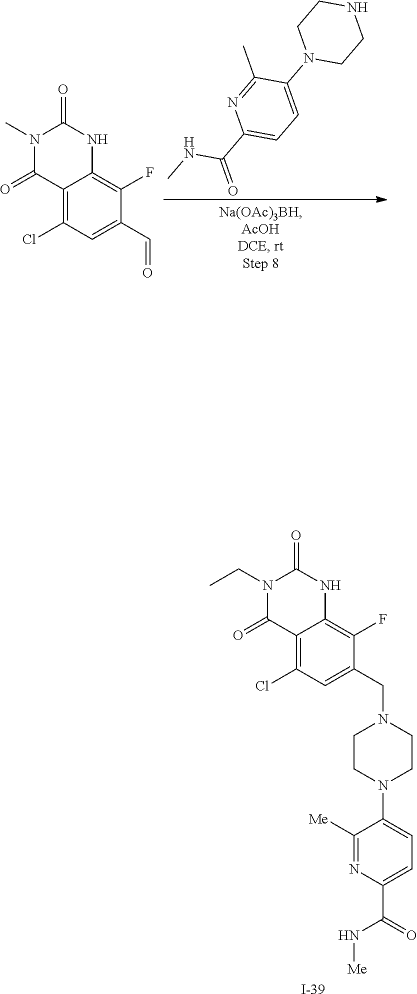

### Step 1: (E)-N-(3-bromo-5-chloro-2-fluorophenyl)-2-(hydroxyimino) acetamide

A mixture of sodium sulfate (32.66 g, 229.96 mmol) in water (280 mL) was treated chloral hydrate (9.51 g, 57.49 mmol) in portions at 0° C., followed by the addition of 3-bromo-5-chloro-2-fluoroaniline hydrochloride (10 g, 38.33 mmol), conc. hydrochloric acid (2.38 g, 65.15 mmol) and hydroxylamine hydrochloride (8.52 g, 122.643 mmol) at 0° C. The reaction mixture was stirred at 80° C. for 5 h. Upon cooling to room temperature, the precipitate was collected by filtration, washed with water (3×400 mL), and dried under vacuum to afford a yellow solid (10 g, 88%). LCMS calculated for C₈H₄BrClFN₂O₂ (M−H)⁻ m/z=292.9; found 292.9; ¹H NMR (300 MHz, CDCl₃) δ 8.57 (s, 1H), 8.46 (dd, J=6.3, 2.4 Hz, 1H), 8.06 (s, 1H), 7.32 (dd, J=5.7, 2.4 Hz, 1H).

### Step 2: 6-bromo-4-chloro-7-fluoroindoline-2,3-dione

A mixture of (E)-N-(3-bromo-5-chloro-2-fluorophenyl)-2-(hydroxyimino) acetamide (10 g, 33.8 mmol) in concentrated sulfuric acid (25 mL) was stirred for 3 h at 80° C. Upon cooling to room temperature, the mixture was poured into ice/water (500 mL). The precipitate was collected by filtration and washed with water (3×100 mL). The resulting residue was purified by silica gel column chromatography, eluted with 50% ethyl acetate in dichloromethane to afford as a brown reddish solid (5.48 g, 58%). LCMS calculated for C₈H₁BrClFNO₂ (M−H)⁻ m/z=275.9; found 275.8; ¹H NMR (300 MHz, DMSO-d₆) δ 11.92 (s, 1H), 7.53 (d, J=5.1 Hz, 1H).

### Step 3:2-Amino-4-bromo-6-chloro-3-fluorobenzoic acid

To a mixture of 6-bromo-4-chloro-7-fluoroindoline-2,3-dione (5.3 g, 19.03 mmol) and sodium hydroxide (7.08 g, 177 mmol) in water (85 mL) was added hydrogen peroxide (30%, 11.2 g, 98.8 mmol) dropwise at 0° C. The reaction mixture was allowed to warm to room temperature, and stirred for 5 h. The mixture was washed with ethyl acetate (100 mL). The aqueous was then neutralized to pH 7 with con. hydrochloric acid at 0° C. The resulting mixture was concentrated under reduced pressure. To the resulting residue was added ethyl acetate (500 mL). The mixture was stirred for 2 h; and then filtered. The filter-cake was washed with ethyl acetate (3×100 mL). The combined filtrate was concentrated under reduced pressure to afford a yellow solid (4.7 g, 92%). LCMS calculated for C₇H₃BrClFNO₂ (M−H)⁻ m/z=265.9; found 265.9.

### Step 4:2-Amino-4-bromo-6-chloro-N-ethyl-3-fluorobenzamide

To a solution of 2-amino-4-bromo-6-chloro-3-fluorobenzoic acid (3.8 g, 14.15 mmol) in N,N-dimethylformamide (40 mL) was added 2-(7-azabenzotriazol-1-yl)-N,N,N′,N′-tetramethyluronium hexafluorophosphate (6.46 g, 16.99 mmol). The mixture was stirred for 15 min at room temperature. Ethylamine hydrochloride (1.73 g, 21.2 mmol) was added, followed by N,N-diisopropylethylamine (5.49 g, 42.4 mmol). The reaction mixture was stirred for additional 2 h; and then diluted with water (500 mL) and extracted with ethyl acetate (3×300 mL). The combined organic layers were washed with brine (3×300 mL), dried over anhydrous sodium sulfate. After filtration, the filtrate was concentrated under reduced pressure. The resulting residue was purified by silica gel column chromatography, eluted with 50% ethyl acetate in petroleum ether to afford as a light-yellow solid (3.5 g, 84%). LCMS calculated for C₉H₁₀BrClFN₂O (M+H)⁺ m/z=295.0; found 295.0.

### Step 5: 7-Bromo-5-chloro-3-ethyl-8-fluoroquinazoline-2,4(1H,3H)-dione

A mixture of N,N-diisopropylethylamine (3.06 g, 23.69 mmol) and 2-amino-4-bromo-6-chloro-N-ethyl-3-fluorobenzamide (3.5 g, 11.84 mmol) in tetrahydrofuran (40 mL) was treated with triphosgene (3.51 g, 11.84 mmol) in portions at 0° C. The reaction was allowed to warm to room temperature and stirred for 1 h. Then the reaction was quenched with sat. ammonium chloride (aq.) at 0° C., and extracted with ethyl acetate (3×100 mL). The combined organic layers were washed with brine (2×100 mL), dried over anhydrous sodium sulfate. After filtration, the filtrate was concentrated under reduced pressure. The resulting residue was purified by silica gel column chromatography, eluted with 50% ethyl acetate in dichloromethane to afford a white solid (3 g, 79%). LCMS calculated for C₁₀H₆BrClFN₂O₂ (M−H)⁻ m/z=318.9; found 318.7. ¹H NMR (400 MHz, CDCl₃) δ 8.59 (s, 1H), 7.43 (d, J=6.0 Hz, 1H), 4.11 (q, J=7.2 Hz, 2H), 1.30 (t, J=7.2 Hz, 3H).

### Step 6: 5-Chloro-3-ethyl-8-fluoro-7-vinylquinazoline-2,4(1H,3H)-dione

A mixture of 7-bromo-5-chloro-3-ethyl-8-fluoroquinazoline-2,4(1H,3H)-dione (2 g, 6.2 mmol), potassium carbonate (2.58 g, 18.66 mmol), 1,1′-bis(diphenylphosphino)ferrocene-palladium(II) dichloride dichloromethane complex (1.01 g, 1.24 mmol) and potassium ethenyltrifluoroboranuide (2.5 g, 18.66 mmol) in dimethyl sulfoxide (20 mL) was stirred at 80° C. for 1 h under nitrogen atmosphere. Upon cooling to room temperature, the reaction was diluted with water (200 mL), and extracted with ethyl acetate (3×200 mL). The combined organic layers were washed with brine (2×200 mL), dried over anhydrous sodium sulfate. After filtration, the filtrate was concentrated under reduced pressure. The resulting residue was purified by silica gel column chromatography, eluted with 20% ethyl acetate in dichloromethane to afford as a yellow solid (1.38 g, 83%). LCMS calculated for C₁₂H₁₁ClFN₂O₂ (M+H)⁺ m/z=269.0; found 268.9.

### Step 7: 5-Chloro-3-ethyl-8-fluoro-2,4-dioxo-1,2,3,4-tetrahydroquinazoline-7-carbaldehyde

A mixture of 5-chloro-3-ethyl-8-fluoro-7-vinylquinazoline-2,4(1H,3H)-dione (1.36 g, 5.06 mmol) and potassium osmate (VI) dihydrate (0.37 g, 1.01 mmol) in dioxane (15 mL) and water (5 mL) was stirred for 0.5 h at room temperature. Sodium periodate (4.33 g, 20.25 mmol) was added. The reaction mixture was stirred for additional 2 h; and then concentrated under reduced pressure. The resulting residue was purified by silica gel column chromatography, eluted with 50% ethyl acetate in dichloromethane to afford as an off-white solid (1 g, 73%). LCMS calculated for C₁₁H₇ClFN₂O₃ (M−H)⁻ m/z=269.0; found 269.0. ¹H NMR (400 MHz, CDCl₃) δ 10.37 (s, 1H), 8.17 (s, 1H), 7.64 (d, J=6.0 Hz, 1H), 4.12 (q, J=7.2 Hz, 2H), 1.31 (t, J=7.2 Hz, 3H).

### Step 8: 5-(4-((5-Chloro-3-ethyl-8-fluoro-2,4-dioxo-1,2,3,4-tetrahydroquinazolin-7-yl)methyl)piperazin-1-yl)-N,6-dimethylpicolinamide (I-39)

A mixture of 5-chloro-3-ethyl-8-fluoro-2,4-dioxo-1,2,3,4-tetrahydroquinazoline-7-carbaldehyde (80 mg, 0.3 mmol) and N,6-dimethyl-5-(piperazin-1-yl)picolinamide (88 mg, 0.33 mmol) in 1,2-dichloroethane (2 mL) was stirred for 30 min. Acetic acid (1.78 mg, 0.03 mmol) was added, followed by sodium triacetoxyborohydride (251 mg, 1.18 mmol). After stirring for additional 16 h at room temperature, the reaction mixture was concentrated under reduced pressure. The residue was purified by silica gel column chromatography, eluted with 7% methanol in dichloromethane. The product was re-purified by reversed-phase flash chromatography with the following conditions: column, C18 silica gel; mobile phase, acetonitrile in water (0.1% trifluoroacetic acid), 5% to 50% gradient over 35 min; detector, UV 254 nm. The fractions were collected, combined and lyophilized to afford the TFA salt of the desired product as a white solid (63 mg). LCMS calculated for C₂₃H₂₇ClFN₆O₃ (M+H)⁺ m/z=489.2; found 489.2. ¹H NMR (300 MHz, CD₃OD) δ 7.93 (d, J=8.4 Hz, 1H), 7.58 (d, J=8.4 Hz, 1H), 7.48 (d, J=6.0 Hz, 1H), 4.55 (s, 2H), 4.09 (q, J=7.2 Hz, 2H), 3.60-3.50 (m, 4H), 3.33-3.25 (m, 4H), 2.97 (s, 3H), 2.62 (s, 3H), 1.28 (t, J=7.2 Hz, 3H).

### Example 40. 5-(4-((5-Cyclopropyl-3-ethyl-8-fluoro-2,4-dioxo-1,2,3,4-tetrahydroquinazolin-7-yl)methyl)piperazin-1-yl)-N,6-dimethylpicolinamide (I-40)

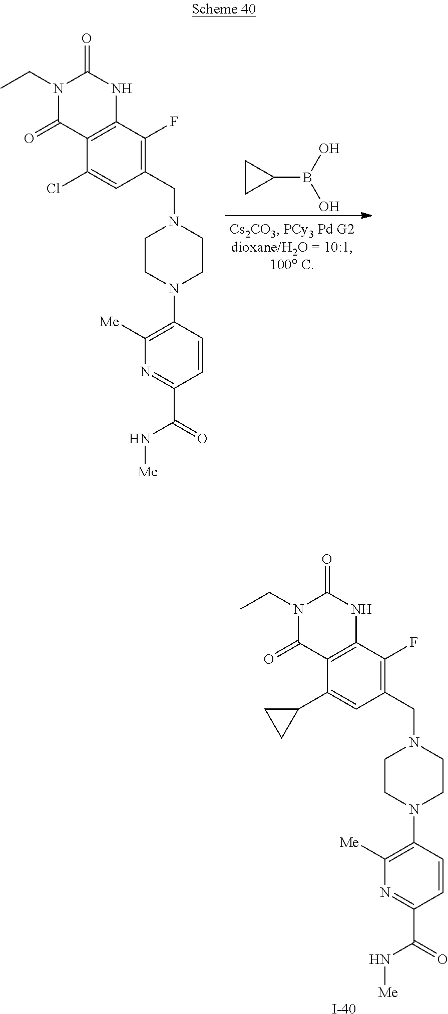

A mixture of 5-{4-[(5-chloro-3-ethyl-8-fluoro-2,4-dioxo-1H-quinazolin-7-yl)methyl]piperazin-1-yl}-N,6-dimethylpyridine-2-carboxamide (Example 39, 100 mg, 0.2 mmol), cyclopropylboronic acid (53 mg, 0.6 mmol), cesium carbonate (200 mg, 0.6 mmol) and (chloro[(tricyclohexylphosphine)-2-(2′-aminobiphenyl)]palladium(II)) (36 mg, 0.06 mmol) in dioxane (3 mL) and water (0.3 mL) was stirred for 16 h at 100° C. under nitrogen atmosphere. Upon cooling to room temperature, the mixture was concentrated under reduced pressure. The resulting residue was purified by silica gel column chromatography, eluted with 10% methanol in dichloromethane to provide the crude product. The crude product was purified by reversed-phase flash chromatography with the following conditions: column, C₁₈ silica gel; mobile phase, acetonitrile in water (0.1% trifluoroacetic acid), 10% to 50% gradient over 25 min, detector, UV 254 nm. The fractions were collected, combined and lyophilized to provide the TFA salt of the desired product as a white solid (70 mg). LCMS calculated for C₂₆H₃₂FN₆O₃ (M+H)⁺ m/z=495.3; found 495.2; ¹H NMR (400 MHz, Methanol-d₄) δ 7.90 (d, J=8.4 Hz, 1H), 7.56 (d, J=8.4 Hz, 1H), 7.02 (d, J=6.4 Hz, 1H), 4.55 (s, 2H), 4.07 (q, J=7.2 Hz, 2H), 3.67-3.52 (m, 6H), 3.26-3.15 (m, 3H), 2.94 (s, 3H), 2.59 (s, 3H), 1.26 (t, J=7.2 Hz, 3H), 1.12-1.06 (m, 2H), 0.82-0.77 (m, 2H).

### Example 41. 5-(4-((3-Ethyl-8-fluoro-5-(hydroxymethyl)-2,4-dioxo-1,2,3,4-tetrahydroquinazolin-7-yl)methyl)piperazin-1-yl)-N,6-dimethylpicolinamide (I-41)

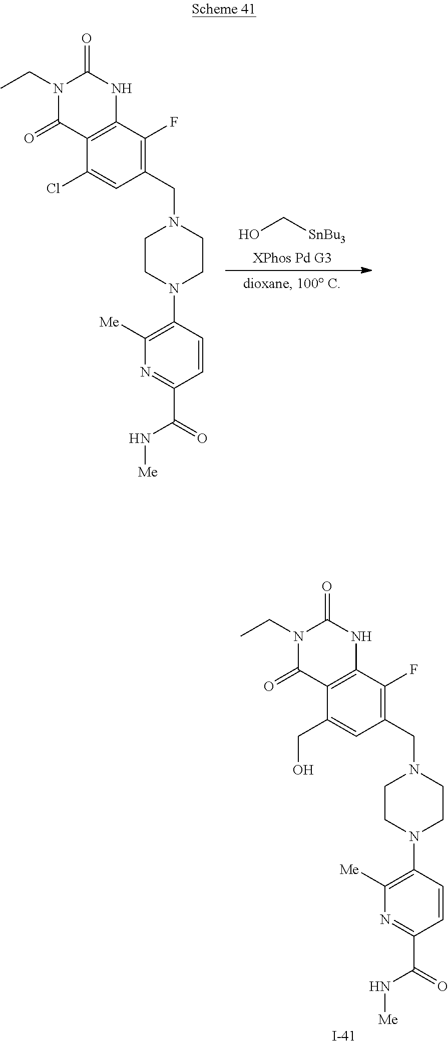

A mixture of 5-{4-[(5-chloro-3-ethyl-8-fluoro-2,4-dioxo-1H-quinazolin-7-yl)methyl]piperazin-1-yl}-N,6-dimethylpyridine-2-carboxamide (Example 39, 200 mg, 0.4 mmol), (tributylstannyl)methanol (197 mg, 0.6 mmol) and methanesulfonato (2-dicyclohexylphosphino-2′,4′,6′-tri-i-propyl-1,1′-biphenyl)(2′-amino-1,1′-biphenyl-2-yl)palladium(II) (69 mg, 0.08 mmol) in 1,4-dioxane (4 mL) was stirred for 2 h at 100° C. under nitrogen atmosphere. Upon cooling to room temperature, the mixture was concentrated under reduced pressure. The resulting residue was purified by silica gel column chromatography, eluted with 7% methanol in dichloromethane to provide the desired product as yellow solid. The product was re-purified by reversed-phase flash chromatography with the following conditions: column, C18 silica gel; mobile phase, acetonitrile in water (0.1% trifluoroacetic acid), 5% to 95% gradient in 22 min; detector, UV 254 nm. The fractions were collected, combined and lyophilized to afford the TFA salt of the desired product as a white solid (68 mg). LCMS calculated for C₂₄H₃₀FN₆O₄ (M+H)⁺ m/z=485.2; found 485.2; ¹H NMR (400 MHz, Methanol-d₄) δ 7.90 (d, J=8.4 Hz, 1H), 7.66 (d, J=6.4 Hz, 1H), 7.57 (d, J=8.4 Hz, 1H), 5.14 (s, 2H), 4.64 (s, 2H), 4.06 (q, J=7.2 Hz, 2H), 3.67-3.52 (m, 6H), 3.26-3.21 (m, 2H), 2.94 (s, 3H), 2.59 (s, 3H), 1.24 (t, J=7.2 Hz, 3H).

### Example 42. 5-(4-((5-(Cyanomethyl)-3-ethyl-8-fluoro-2,4-dioxo-1,2,3,4-tetrahydroquinazolin-7-yl)methyl)piperazin-1-yl)-N,6-dimethylpicolinamide (I-42)

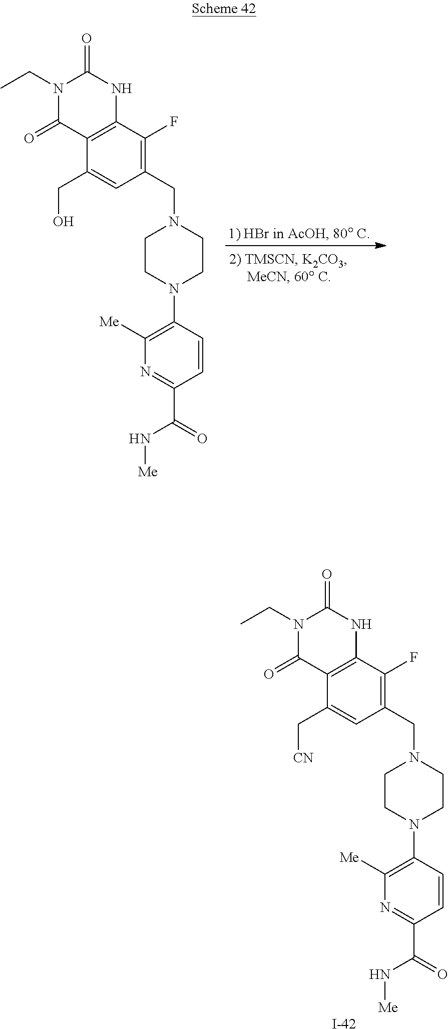

To 5-(4-{[3-ethyl-8-fluoro-5-(hydroxymethyl)-2,4-dioxo-1H-quinazolin-7-yl]methyl}piperazin-1-yl)-N,6-dimethylpyridine-2-carboxamide (Example 41, 103 mg, 0.21 mmol) was added hydrogen bromide (33 wt. % solution in glacial acid, 1 mL) at room temperature. The reaction mixture was heated at 80° C. for 1 h. Upon cooling to room temperature, the reaction was concentrated under reduced pressure. The resulting mixture was diluted with water (20 mL). The mixture was basified to pH 9 with potassium carbonate. The resulting mixture was extracted with ethyl acetate (3×20 mL). The combined organic layers were dried over anhydrous sodium sulfate. After filtration, the filtrate was concentrated under reduced pressure. To the resulting residue was charged acetonitrile (2 mL), followed by trimethylsilyl cyanide (32 mg, 0.32 mmol) and potassium carbonate (88 mg, 0.64 mmol). The resulting mixture was stirred at 60° C. for additional 16 h. Upon cooling to room temperature, the resulting mixture was concentrated under reduced pressure. The resulting residue was purified by silica gel column chromatography, eluted with 7% methanol in dichloromethane to provide crude product. The crude product was purified by reversed-phase flash chromatography with the following conditions: column, C18 silica gel; mobile phase, acetonitrile in water (0.1% trifluoroacetic acid), 5% to 95% gradient over 25 min; detector, UV 254 nm. The fractions were collected, combined and lyophilized to provide the TFA salt of the desired product as a white solid (56 mg). LCMS calculated for C₂₅H₂₉FN₇O₃ (M+H)⁺ m/z=494.2; found 494.2; ¹H NMR (400 MHz, Methanol-d₄) δ 7.90 (d, J=8.4 Hz, 1H), 7.57 (d, J=8.4 Hz, 1H), 7.46 (d, J=6.0 Hz, 1H), 4.64 (s, 2H), 4.46 (s, 2H), 4.06 (q, J=7.2 Hz, 2H), 3.62-3.43 (m, 6H), 3.24-3.06 (m, 2H), 2.94 (d, J=0.8 Hz, 3H), 2.60 (s, 3H), 1.25 (t, J=7.2 Hz, 3H).

### Example 43: 5-(4-((5-Chloro-3-methyl-2,4-dioxo-1,2,3,4-tetrahydroquinazolin-7-yl)methyl)piperazin-1-yl)-N-ethyl-6-methylpicolinamide (I-43)

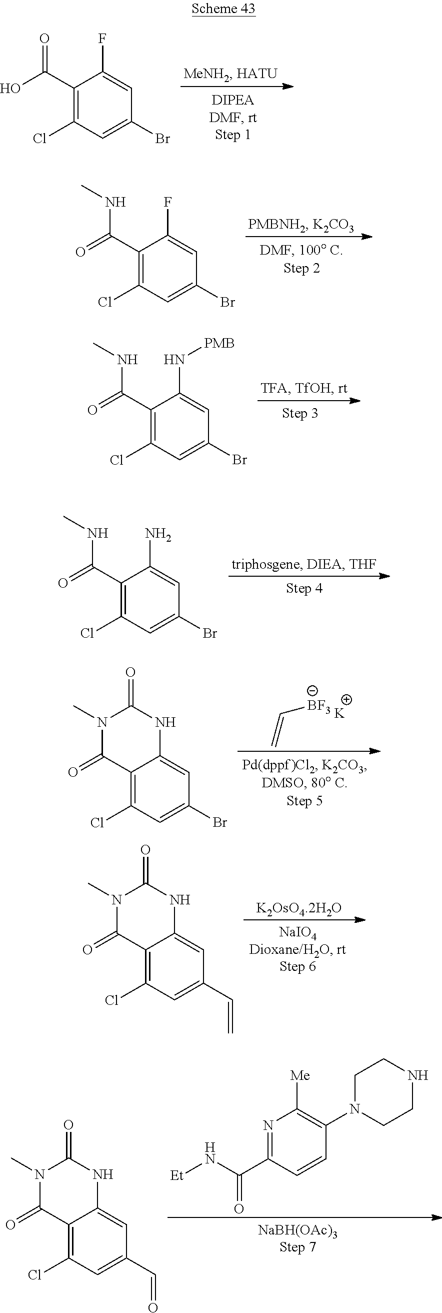

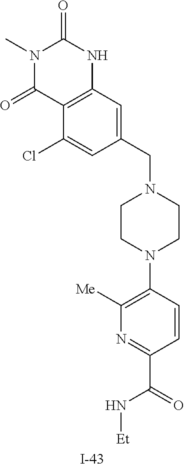

### Step 1: 4-Bromo-2-chloro-6-fluoro-N-methylbenzamide

A mixture of 4-bromo-2-chloro-6-fluorobenzoic acid (9 g, 35.5 mmol) and 2-(7-azabenzotriazol-1-yl)-N,N,N′,N′-tetramethyluronium hexafluorophosphate (16.2 g, 42.6 mmol) in N,N-dimethylformamide (90 mL) was stirred at room temperature for 30 min. Methylamine (2 M in tetrahydrofuran, 19.5 mL, 39.1 mmol) was added, followed by N,N-diisopropylethylamine (13.8 g, 106.5 mmol). The reaction mixture was stirred for additional 2 h; and then diluted with water (1000 mL), extracted with ethyl acetate (3×500 mL). The combined organic layers were washed with brine (2×500 mL), dried over anhydrous sodium sulfate. After filtration, the filtrate was concentrated under reduced pressure. The resulting residue was purified by silica gel column chromatography, eluted with 50% ethyl acetate in petroleum ether to afford the desired product as a white solid (8.03 g, 85%). LCMS calculated for C₈H₇BrClFNO (M+H)⁺ m/z=265.9; found 265.8. ¹H NMR (400 MHz, CDCl₃) δ 7.39 (d, J=1.6 Hz, 1H), 7.23 (dd, J=8.4, 1.6 Hz, 1H), 5.87 (s, 1H), 3.02 (d, J=4.8 Hz, 3H). ¹⁹F NMR (377 MHz, CDCl₃) δ −110.81.

### Step 2: 4-Bromo-2-chloro-6-((4-methoxybenzyl)amino)-N-methylbenzamide

A mixture of 4-bromo-2-chloro-6-fluoro-N-methylbenzamide (3.6 g, 13.36 mmol), potassium carbonate (7.4 g, 53.4 mmol) and 4-methoxybenzylamine (3.67 g, 26.7 mmol) in N,N-dimethylformamide (36 mL) was stirred at 100° C. for 16 h. Upon cooling to room temperature, the reaction mixture was diluted with water (400 mL), and extracted with ethyl acetate (3×400 mL). The combined organic layers were washed with brine (400 mL), dried over anhydrous sodium sulfate. After filtration, the filtrate was concentrated under reduced pressure. The resulting residue was purified by reversed-phase flash chromatography with the following conditions: column, C₁₈ silica gel; mobile phase, methanol in water (0.1% trifluoroacetic acid), 50% to 95% gradient in 30 min; detector, UV 254 nm. The fractions were collected, combined and lyophilized to provide the desired product as a brown solid (4.45 g, 86%). LCMS calculated for C₁₆H₁₇BrClN₂O₂ (M+H)⁺ m/z=383.0; found 383.0. ¹H NMR (400 MHz, CDCl₃) δ 7.25-7.20 (m, 2H), 6.91-6.85 (m, 2H), 6.82 (d, J=1.6 Hz, 1H), 6.67 (d, J=1.6 Hz, 1H), 6.07 (s, 1H), 4.22 (s, 2H), 3.80 (s, 3H), 2.99 (d, J=4.8 Hz, 3H).

### Step 3:2-Amino-4-bromo-6-chloro-N-methylbenzamide

A mixture of 4-bromo-2-chloro-6-((4-methoxybenzyl)amino)-N-methylbenzamide (4.3 g, 9.9 mmol) in trifluoroacetic acid (8 mL) was treated with trifluoromethanesulfonic acid (2.5 mL) dropwise at room temperature. The reaction mixture was stirred at room temperature for 2 h; and then poured into a saturated sodium bicarbonate aqueous solution (200 mL). The mixture was extracted with dichloromethane (3×200 mL). The combined organic layers were washed with brine (200 mL), dried over anhydrous sodium sulfate. After filtration, the filtrate was concentrated under reduced pressure. The resulting residue was purified by silica gel column chromatography, eluted with 50% ethyl acetate in petroleum ether to afford the desired product as a yellow solid (2.26 g, 86%). LCMS calculated for C₈H₉BrClN₂O (M+H)⁺ m/z=263.0; found 262.9; ¹H NMR (400 MHz, CDCl₃) δ 6.87 (d, J=1.6 Hz, 1H), 6.75 (d, J=1.6 Hz, 1H), 6.08 (s, 1H), 4.73 (s, 2H), 3.01 (d, J=4.8 Hz, 3H).

### Step 4: 7-Bromo-5-chloro-3-methylquinazoline-2,4(1H,3H)-dione

A mixture of 2-amino-4-bromo-6-chloro-N-methylbenzamide (2.06 g, 7.74 mmol) and N,N-diisopropylethylamine (3 g, 23.2 mmol) in tetrahydrofuran (40 mL) was treated triphosgene (2.76 g, 9.3 mmol) in portions at 0° C. The reaction mixture was stirred at room temperature for 40 min; and then quenched with a saturated ammonium chloride aqueous solution at 0° C. The mixture was extracted with ethyl acetate (3×200 mL). The combined organic layers were washed with brine (300 mL), dried over anhydrous sodium sulfate. After filtration, the filtrate was concentrated under reduced pressure to afford the desired product as a yellow solid (2.57 g). The crude product was used in the next step directly without further purification. LCMS calculated for C₉H₇BrClN₂O₂ (M+H)⁺ m/z=288.9; found 288.8. ¹H NMR (400 MHz, CDCl₃) δ 9.70 (s, 1H), 7.41 (d, J=1.6 Hz, 1H), 7.21 (d, J=1.6 Hz, 1H), 3.44 (s, 3H).

### Step 5: 5-Chloro-3-methyl-7-vinylquinazoline-2,4(1H,3H)-dione

A mixture of 7-bromo-5-chloro-3-methylquinazoline-2,4(1H,3H)-dione (2.47 g, 8.53 mmol), potassium ethenyltrifluoroboranuide (3.43 g, 25.6 mmol), 1,1′-Bis(diphenylphosphino)ferrocene-palladium(II) dichloride dichloromethane complex (695 mg, 0.85 mmol) and potassium carbonate (3.54 g, 25.6 mmol) in dimethyl sulfoxide (30 mL) was stirred at 80° C. for 16 h under nitrogen atmosphere. Upon cooling to room temperature, the reaction mixture was diluted with water (400 mL), and extracted with ethyl acetate (3×400 mL). The combined organic layers were washed with brine (400 mL), dried over anhydrous sodium sulfate. After filtration, the filtrate was concentrated under reduced pressure. The resulting residue was purified by silica gel column chromatography, eluted with 50% ethyl acetate in dichloromethane to afford the desired product as a light-yellow solid (1.17 g, 58%). LCMS calculated for C₁₁H₁₀ClN₂O₂ (M+H)⁺ m/z=237.0; found 237.2. ¹H NMR (400 MHz, DMSO-d₆) δ 11.53 (s, 1H), 7.39 (d, J=1.6 Hz, 1H), 7.11 (d, J=1.6 Hz, 1H), 6.74 (dd, J=17.6, 10.9, 1H), 6.01 (d, J=17.6, 1H), 5.53 (d, J=10.9 Hz, 1H), 3.21 (s, 3H).

### Step 6: 5-Chloro-3-methyl-2,4-dioxo-1,2,3,4-tetrahydroquinazoline-7-carbaldehyde

A mixture of 5-chloro-3-methyl-7-vinylquinazoline-2,4(1H,3H)-dione (960 mg, 4.06 mmol) in dioxane (25 mL) and water (5 mL) were treated with potassium osmate (VI) dihydrate (299 mg, 0.8 mmol) at room temperature. The mixture was stirred at room temperature for 30 min; and then sodium periodate (3.47 g, 16.2 mmol) was added. The reaction mixture was stirred for additional 1 h; and then concentrated under reduced pressure. The resulting residue was purified by silica gel column chromatography, eluted with 25% ethyl acetate in dichloromethane to afford the desired product as a green solid (870 mg, 90%). LCMS calculated for C₁₀H₆ClN₂O₃ (M−H)⁻ m/z=237.0; found 236.9. ¹H NMR (400 MHz, DMSO-d₆) δ 11.83 (s, 1H), 10.00 (s, 1H), 7.67 (d, J=1.6 Hz, 1H), 7.58 (d, J=1.6 Hz, 1H), 3.23 (s, 3H).

### Step 7: 5-(4-((5-Chloro-3-methyl-2,4-dioxo-1,2,3,4-tetrahydroquinazolin-7-yl)methyl)piperazin-1-yl)-N-ethyl-6-methylpicolinamide (I-43)

A solution of 5-chloro-3-methyl-2,4-dioxo-1,2,3,4-tetrahydroquinazoline-7-carbaldehyde (100 mg, 0.42 mmol) in 1,2-dichloroethane (3 mL) was treated with N-ethyl-6-methyl-5-(piperazin-1-yl)pyridine-2-carboxamide (135 mg, 0.55 mmol) at room temperature. The mixture was stirred for 1 h. The reaction was cooled to 0° C., and sodium triacetoxyborohydride (133 mg, 0.63 mmol) was charged in portions. The reaction mixture was stirred for additional 1 h; and then concentrated under reduced pressure. The resulting residue was purified by silica gel column chromatography, eluted with 10% methanol in dichloromethane to provide the desired product. The product was re-purified by reversed-phase flash chromatography with the following conditions: column, C18 silica gel; mobile phase, acetonitrile in water (0.1% trifluoroacetic acid), 5% to 95% gradient in 20 min; detector, UV 254 nm. The fractions were collected, combined and lyophilized to provide the TFA salt of the desired product as a white solid (55 mg). LCMS calculated for C₂₃H₂₈ClN₆O₃ (M+H)⁺ m/z=471.2; found 471.2. ¹H NMR (400 MHz, CD₃OD) δ 7.91 (d, J=8.4 Hz, 1H), 7.57 (d, J=8.4 Hz, 1H), 7.46 (d, J=1.6 Hz, 1H), 7.27 (d, J=1.6 Hz, 1H), 4.48 (s, 2H), 3.58-3.49 (m, 4H), 3.43 (q, J=7.2 Hz, 2H), 3.36 (s, 3H), 3.31-3.19 (m, 4H), 2.60 (s, 3H), 1.22 (t, J=7.2 Hz, 3H).

### Example 44: 5-(4-((3-ethyl-2,4-dioxo-1,2,3,4-tetrahydroquinazolin-7-yl)methyl)piperazin-1-yl)-N,6-bis(methyl-d3)picolinamide (I-44)

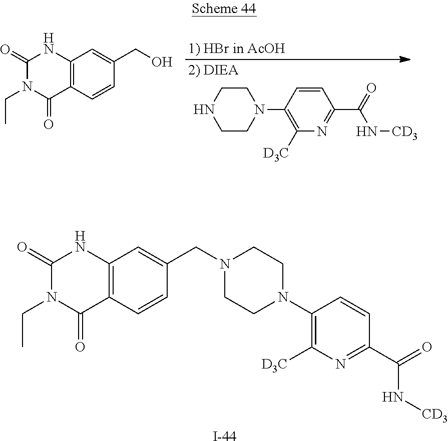

The trifluoroacetic acid (TFA) salt of the desired compound was prepared according to the procedure described in Example 16, using N,6-bis(methyl-d₃)-5-(piperazin-1-yl)picolinamide instead of N,6-dimethyl-5-(piperazin-1-yl)pyridine-2-carboxamide hydrochloride as the starting material. LCMS calculated for C₂₃H₂₃D₆N₆O₃ (M+H)⁺ m/z=443.3; found 443.1; ¹H NMR (400 MHz, CD₃OD) δ 8.17 (d, J=8.0 Hz, 1H), 7.91 (d, J=8.4 Hz, 1H), 7.57 (d, J=8.4 Hz, 1H), 7.39 (dd, J=8.0, 1.6 Hz, 1H), 7.34 (s, 1H), 4.53 (s, 2H), 4.08 (q, J=7.2 Hz, 2H), 3.63-3.47 (m, 6H), 3.23-3.08 (m, 2H), 1.25 (t, J=7.2 Hz, 3H).

### Example 45: 5-[4-[(12-ethyl-11-oxo-2,3,10,12-tetrazatricyclo[7.3.1.05,13]trideca-1,3,5,7,9 (13)-pentaen-7-yl)methyl]piperazin-1-yl]-N,6-dimethyl-pyridine-2-carboxamid (I-45)

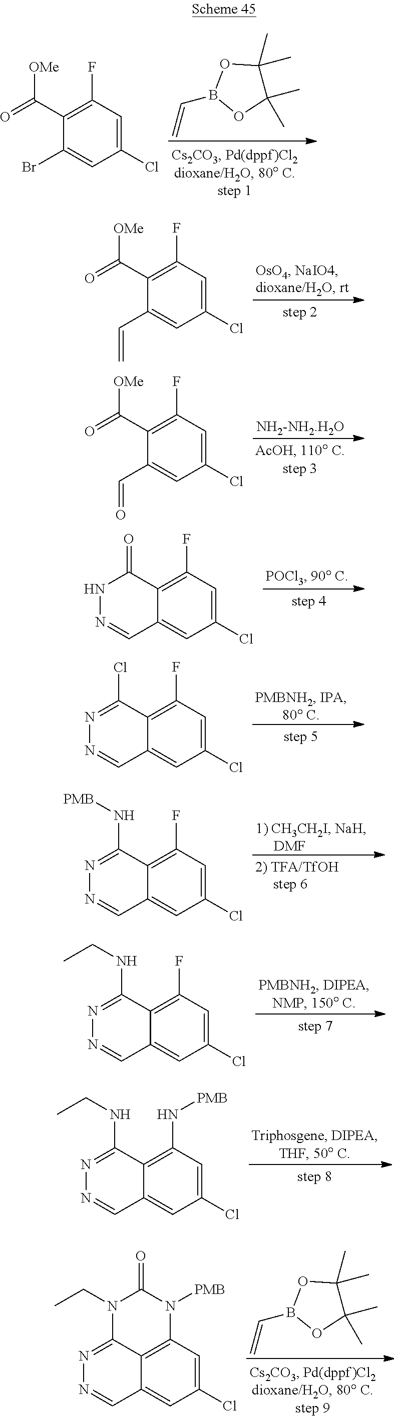

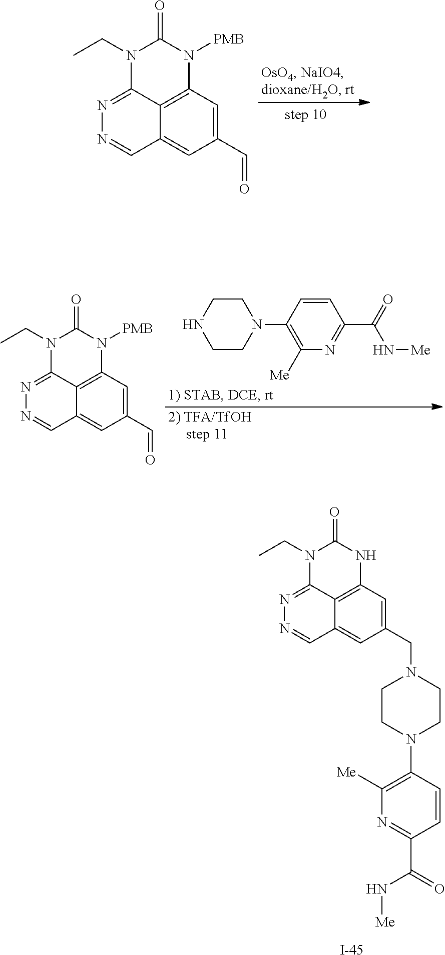

### Step 1: Methyl 4-chloro-2-fluoro-6-vinyl-benzoate

A mixture of methyl 2-bromo-4-chloro-6-fluoro-benzoate (3.50 g, 13.09 mmol), 4,4,5,5-tetramethyl-2-vinyl-1,3,2-dioxaborolane (2.42 g, 15.70 mmol), dipotassium carbonate (5.43 g, 39.26 mmol) and [1,1′-bis(diphenylphosphino)ferrocene]dichloropalladium(II) (1.07 g, 1.31 mmol) in 40 mL 1,4-dioxane and 10 mL water was bubbled with N₂ for 5 min. The resulting reaction mixture was stirred at 80° C. for 4 h. After cooling to room temperature, the reaction mixture was diluted with water and extracted with ethyl acetate (2×50 mL). The combined organics were dried with anhydrous sodium sulfate, filtered, and concentrated under reduced pressure. The residue was purified by silica gel column chromatography, eluted with hexanes, DCM and ethyl acetate to provide the desired product (2.55 g, 90%).

### Step 2: Methyl 4-chloro-2-fluoro-6-formyl-benzoate

To a mixture of methyl 4-chloro-2-fluoro-6-vinyl-benzoate (2.65 g, 12.35 mmol) in 1,4-dioxane (100 mL) and water (20 mL) was added sodium periodate (7.92 g, 37.04 mmol). Osmium tetroxide (4 wt % in H₂O, 4.00 g, 0.63 mmol) was then added dropwise. After stirring at room temperature for 4 h, the reaction mixture was diluted with water (100 mL) and extracted with ethyl acetate (3×100 mL). The combined organic layers were washed with brine (100 mL), dried over anhydrous sodium sulfate. After filtration, the filtrate was concentrated under reduced pressure. The resulting residue was purified by silica gel column chromatography, eluted with DCM and ethyl acetate to provide the desired product (2.34 g, 87.5%).

### Step 3: 6-Chloro-8-fluoro-2H-phthalazin-1-one

To a stirred solution of methyl 4-chloro-2-fluoro-6-formyl-benzoate (2.34 g, 10.80 mmol) in 50 mL AcOH was added hydrazine hydrate (0.81 g, 16.21 mmol) dropwise. Then the reaction mixture was stirred at 110° C. for 2h. Upon cooling to room temperature, the reaction mixture was concentrated under reduced pressure. Then the crude product was washed with sat. NaHCO₃(aq) and extracted with EtOAc 3 times. The combined organics were washed with sat. NaCl (aq), dried over anhydrous sodium sulfate, filtered, and concentrated under reduced pressure. The resulting residue was purified by silica gel column chromatography, eluted with DCM and ethyl acetate to provide the desired product (1.45 g, 67%). LCMS calculated for C₈H₅ClFN₂O (M+H)⁺ m/z=199.0; found 199.0.

### Step 4:1,6-Dichloro-8-fluoro-phthalazine

A mixture of 6-chloro-8-fluoro-2H-phthalazin-1-one (750 mg, 3.78 mmol) and phosphoryl trichloride (3.00 mL, 32.09 mmol) was stirred at 90° C. for 2 h. The reaction was cooled to room temperature and concentrated under reduced pressure. The residue was dissolved in DCM, washed with saturated sodium bicarbonate and brine. The separated organic layer was dried over anhydrous sodium sulfate, filtered, and concentrated under reduced pressure. The resulting residue was purified by silica column chromatography (0˜50% EtOAc/DCM) to afford the desired product (610 mg, 80%). LCMS calculated for C₈H₄C₁₂FN₂ (M+H)⁺ m/z=217.0; found 217.1.

### Step 5: 6-Chloro-8-fluoro-N-[(4-methoxyphenyl)methyl]phthalazin-1-amine

To a stirred solution of 1,6-dichloro-8-fluoro-phthalazine (600.00 mg, 2.76 mmol) in 10 mL IPA was added 4-methoxybenzylamine (455 mg, 3.32 mmol) and the resulting mixture was stirred at 80° C. for 16 h. After cooling to room temperature, the reaction mixture was concentrated. The resulting residue was purified by silica column chromatography (5˜75% EtOAc/Hexane) to afford the desired product (615 mg, 70%). LCMS calculated for C₁₆H₁₄ClFN₃O (M+H)⁺ m/z=318.1; found 318.0.

### Step 6: 6-Chloro-N-ethyl-8-fluoro-phthalazin-1-amine

To a stirred solution of 6-chloro-8-fluoro-N-[(4-methoxyphenyl)methyl]phthalazin-1-amine (600 mg, 1.89 mmol) in 10 mL DMF was added sodium hydride (67.97 mg, 2.83 mmol) at 0° C. and the resulting mixture was stirred for 30 min. Iodoethane (441.77 mg, 2.83 mmol) was then added, the resulting mixture was allowed to warm to room temperature and stirred for 2 h. The reaction was quenched with sat. NH₄Cl (aq) and extracted with EtOAc (3×). The combined organic layers were dried over anhydrous sodium sulfate, filtered and concentrated under reduced pressure. The residue was treated with 2,2,2-trifluoroacetic acid (1.45 mL) and trifluoromethanesulfonic acid (0.17 mL). The mixture was stirred at room temperature for 1 h, and then concentrated under reduced pressure. The resulting residue was dissolved in DCM, washed with sat. NaHCO₃(aq) and brine. The separated organic layer was dried over anhydrous sodium sulfate, filtered, and concentrated under reduced pressure to afford the desired product (410 mg) which was directly used in the next step without further purification. LCMS calculated for C₁₀H₁₀ClFN₃ (M+H)⁺ m/z=226.1; found 226.0.

### Step 7: 6-Chloro-N₁-ethyl-N₈-[(4-methoxyphenyl)methyl]phthalazine-1,8-diamine

To a stirred solution of 6-chloro-N-ethyl-8-fluoro-phthalazin-1-amine (500 mg, 2.22 mmol) in 10 mL NMP was added 4-methoxybenzylamine (607.9 mg, 4.43 mmol) and N-ethyl-N-isopropyl-propan-2-amine (859.14 mg, 6.65 mmol). The resulting mixture was stirred at 150° C. for 2 h. The reaction was cooled to room temperature and diluted with water. The resulting precipitate was collected by filtration to afford the desired product as a pale-yellow solid (602 mg, 79%). LCMS calculated for C₁₈H₂₀ClN₄O (M+H)⁺ m/z=343.1; found 343.1.

### Step 8: 7-Chloro-12-ethyl-10-[(4-methoxyphenyl)methyl]-2,3,10,12-tetrazatricyclo[7.3.1.05,13]trideca-1,3,5 (13), 6,8-pentaen-11-one

To a mixture of 6-chloro-N₁-ethyl-N₈-[(4-methoxyphenyl)methyl]phthalazine-1,8-diamine (600 mg, 1.75 mmol) and N,N-diisopropylethylamine (531.3 mg, 4.11 mmol) in 10 mL THF was added bis(trichloromethyl) carbonate (363.55 mg, 1.23 mmol) at 0° C. The mixture was stirred at 50° C. for 1 h. After cooling to room temperature, the reaction was quenched with saturated aqueous NH₄Cl (20 mL), extracted with EtOAc (3×20 mL). The organic layers were dried over anhydrous sodium sulfate. After filtration, the filtrate was concentrated under reduced pressure. The resulting residue was purified by silica gel column chromatography, eluted with 0˜60% ethyl acetate in hexanes to provide the desired product (525 mg, 81%). LCMS calculated for C₁₉H₁₈ClN₄O₂ (M+H)⁺ m/z=369.1; found 369.1.

### Step 9:12-Ethyl-10-[(4-methoxyphenyl)methyl]-7-vinyl-2,3,10,12-tetrazatricyclo[7.3.1.05,13]trideca-1,3,5 (13), 6,8-pentaen-11-one

A mixture of 7-chloro-12-ethyl-10-[(4-methoxyphenyl)methyl]-2,3,10,12-tetrazatricyclo[7.3.1.05,13]trideca-1,3,5 (13),6,8-pentaen-11-one (550 mg, 1.49 mmol), 4,4,5,5-tetramethyl-2-vinyl-1,3,2-dioxaborolane (459.35 mg, 2.98 mmol), cesium carbonate (1457.64 mg, 4.47 mmol) and [1,1′-bis(diphenylphosphino)ferrocene]dichloropalladium(II) (109.12 mg, 0.15 mmol) in 10 mL 1,4-dioxane and 2 mL water was bubbled with N₂ for 5 min. The resulting reaction mixture was stirred at 80° C. for 4 h. After cooling to room temperature, the reaction mixture was diluted with water and extracted with ethyl acetate (2×20 mL). The combined organics were dried with anhydrous sodium sulfate, filtered, and concentrated under reduced pressure. The resulting residue was purified by silica gel column chromatography, eluted with hexanes, DCM and ethyl acetate to provide the desired product (450 mg, 83%). LCMS calculated for C₂₁H₂₁N₄O₂ (M+H)⁺ m/z=361.2; found 361.1.

### Step 10:12-Ethyl-10-[(4-methoxyphenyl)methyl]-11-oxo-2,3,10,12-tetrazatricyclo[7.3.1.05,13]trideca-1,3,5 (13), 6,8-pentaene-7-carbaldehyde

To a mixture of 12-ethyl-10-[(4-methoxyphenyl)methyl]-7-vinyl-2,3,10,12-tetrazatricyclo[7.3.1.05,13]trideca-1,3,5 (13),6,8-pentaen-11-one (500 mg, 1.39 mmol) in 1,4-dioxane (30 mL) and water (10 mL) was added sodium periodate (890.2 mg, 4.16 mmol). Osmium tetroxide (4 wt % in H2O, 444.9 mg, 0.07 mmol) was added dropwise. After stirring at room temperature for 4 h, the reaction mixture was diluted with water (50 mL) and extracted with ethyl acetate (3×50 mL). The combined organic layers were washed with brine (50 mL), dried over anhydrous sodium sulfate. After filtration, the filtrate was concentrated under reduced pressure. The resulting residue was purified by silica gel column chromatography, eluted with DCM and ethyl acetate to provide the desired product (275 mg, 54%). LCMS calculated for C₂₀H₁₉N₄O₃ (M+H)⁺ m/z=363.1; found 363.1.

### Step 11: 5-[4-[(12-Ethyl-11-oxo-2,3,10,12-tetrazatricyclo[7.3.1.05,13]trideca-1,3,5,7,9 (13)-pentaen-7-yl)methyl]piperazin-1-yl]-N,6-dimethyl-pyridine-2-carboxamide (I-45)

To a stirred solution of 12-ethyl-10-[(4-methoxyphenyl)methyl]-11-oxo-2,3,10,12-tetrazatricyclo[7.3.1.05,13]trideca-1,3,5 (13),6,8-pentaene-7-carbaldehyde (30 mg, 0.08 mmol) and N,6-dimethyl-5-piperazin-1-yl-pyridine-2-carboxamide (23 mg, 0.10 mmol) in 1 mL DCE was added sodium triacetoxyborohydride (52.6 mg, 0.25 mmol) at room temperature. After stirring at room temperature for 2 h, the reaction mixture was diluted with sat. NaHCO₃(aq) and extracted with DCM. The combined organics were washed with brine, dried over anhydrous sodium sulfate, filtered, and concentrated under reduced pressure. The residue was treated with 2,2,2-trifluoroacetic acid (1.50 mL) and trifluoromethanesulfonic acid (0.40 mL). The mixture was stirred at 50° C. for 1 h, and then concentrated. The resulting residue was purified by prep-HPLC (Column: Sunfire prep C18 column; mobile phase A: water (0.05% trifluoroacetic acid), mobile Phase B: acetonitrile; flow rate: 60 mL/min; gradient: 5% B to 30% B over 7 min). Eluted fractions were collected and lyophilized to afford the TFA salt of the desired product. LCMS calculated for C₂₄H₂₉N₈O₂ (M+H)⁺ m/z=461.2; found 461.1.

### Example 46: 5-(4-((3-Ethyl-8-fluoro-5-(methoxymethyl)-2,4-dioxo-1,2,3,4-tetrahydroquinazolin-7-yl)methyl)piperazin-1-yl)-N,6-dimethylpicolinamide (I-46)

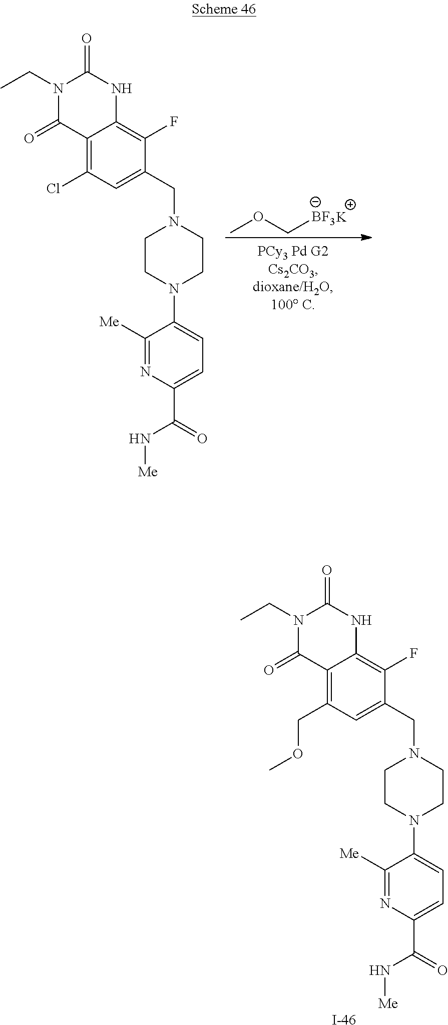

A mixture of 5-(4-((5-chloro-3-ethyl-8-fluoro-2,4-dioxo-1,2,3,4-tetrahydroquinazolin-7-yl)methyl)piperazin-1-yl)-N,6-dimethylpicolinamide (Example 39, 80 mg, 0.16 mmol), potassium trifluoro(methoxymethyl) boranuide (49.7 mg, 0.33 mmol), PCy3 Pd G2 (29 mg, 0.05 mmol) and cesium carbonate (107 mg, 0.33 mmol) in dioxane (2 mL) and water (0.4 mL) was stirred at 100° C. for 16 h under nitrogen atmosphere. Upon cooling to room temperature, the reaction mixture was concentrated under reduced pressure and the resulting residue was purified by silica gel column chromatography, eluted with 7% methanol in dichloromethane. The product was re-purified by reversed-phase flash chromatography with the following conditions: column, C18 silica gel; mobile phase, acetonitrile in water (0.1% trifluoroacetic acid), 5% to 50% gradient over 30 min; detector, UV 254 nm. The fractions were collected, combined and lyophilized to afford the TFA salt of the desired product as a white solid (46 mg). LCMS calculated for C₂₅H₃₂FN₆O₄ (M+H)⁺ m/z=499.2; found 499.3; ¹H NMR (400 MHz, CD₃OD) δ 7.90 (d, J=8.4 Hz, 1H), 7.64 (d, J=6.8 Hz, 1H), 7.56 (d, J=8.4 Hz, 1H), 5.03 (s, 2H), 4.62 (s, 2H), 4.05 (q, J=7.2 Hz, 2H), 3.67-3.50 (m, 7H), 3.30-3.13 (m, 4H), 2.94 (s, 3H), 2.59 (s, 3H), 1.24 (t, J=7.2 Hz, 3H).

### Example 47: 5-(4-((5-(Difluoromethyl)-3-methyl-2,4-dioxo-1,2,3,4-tetrahydroquinazolin-7-yl)methyl)piperazin-1-yl)-N,6-dimethylpicolinamide (I-47)

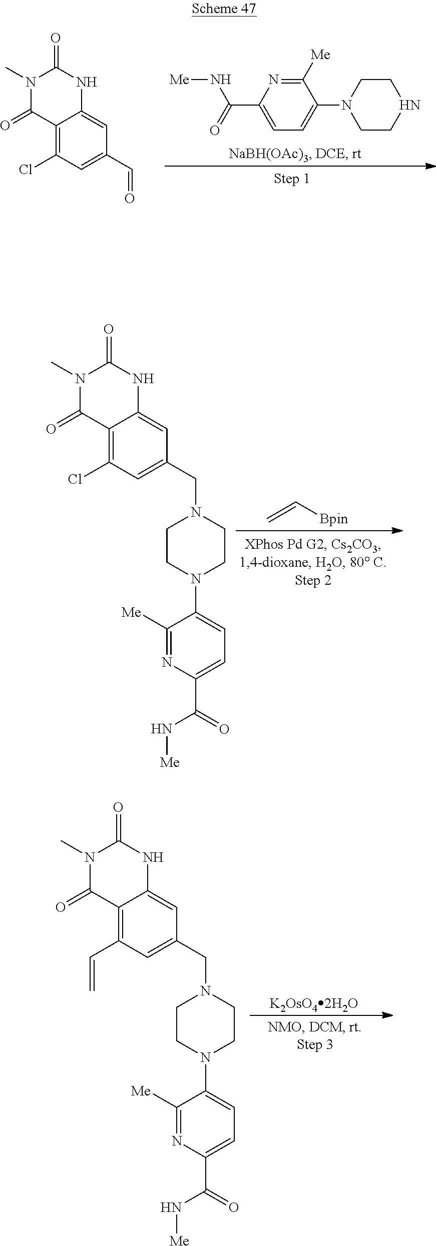

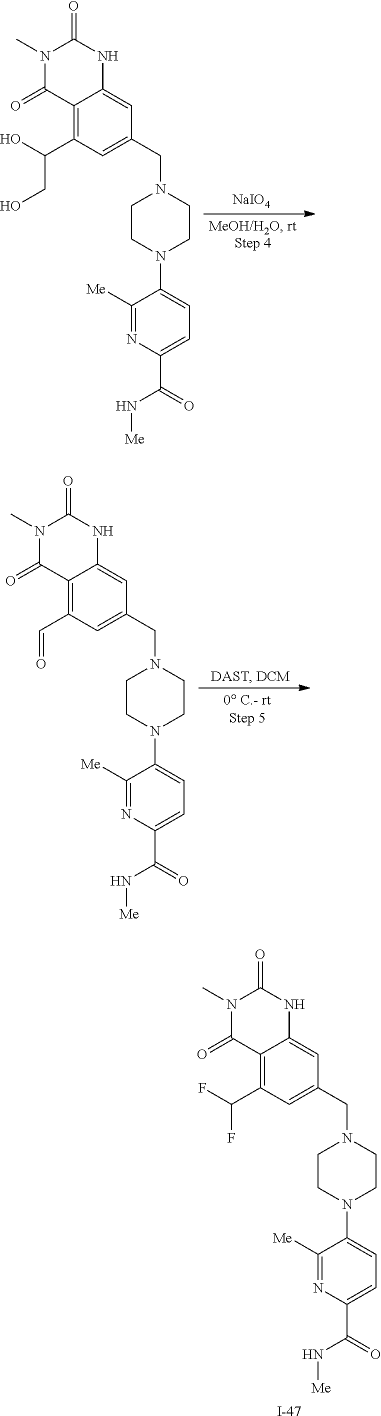

### Step 1: 5-(4-((5-Chloro-3-methyl-2,4-dioxo-1,2,3,4-tetrahydroquinazolin-7-yl)methyl)piperazin-1-yl)-N,6-dimethylpicolinamide

A mixture of 5-chloro-3-methyl-2,4-dioxo-1,2,3,4-tetrahydroquinazoline-7-carbaldehyde (Example 43, Step 6:660 mg, 2.77 mmol) and N,6-dimethyl-5-(piperazin-1-yl)pyridine-2-carboxamide (777.6 mg, 3.32 mmol) in 1,2-dichloroethane (10 mL) was stirred for 16 h at room temperature. The reaction was cooled to 0° C. and treated with sodium triacetoxyborohydride (1.17 g, 5.53 mmol). The reaction mixture was stirred for additional 2 h; and then concentrated under reduced pressure. The resulting residue was purified by silica gel column chromatography, eluted with 7% methanol in dichloromethane to afford a yellow solid (775 mg, 61%). LCMS calculated for C₂₂H₂₆ClN₆O₃ (M+H)⁺ m/z=457.2; found 457.2; ¹H NMR (400 MHz, CDCl₃) δ 9.63 (s, 1H), 8.05-7.95 (m, 2H), 7.35 (d, J=8.4 Hz, 1H), 7.30 (d, J=1.2 Hz, 1H), 7.13 (s, 1H), 3.62 (s, 2H), 3.45 (s, 3H), 3.14-2.92 (m, 7H), 2.79-2.59 (m, 4H), 2.52 (s, 3H).

### Step 2: N,6-Dimethyl-5-(4-((3-methyl-2,4-dioxo-5-vinyl-1,2,3,4-tetrahydroquinazolin-7-yl)methyl)piperazin-1-yl)picolinamide

A mixture of 5-(4-((5-chloro-3-methyl-2,4-dioxo-1,2,3,4-tetrahydroquinazolin-7-yl)methyl)piperazin-1-yl)-N,6-dimethylpicolinamide (750 mg, 1.64 mmol), 2-ethenyl-4,4,5,5-tetramethyl-1,3,2-dioxaborolane (303 mg, 1.97 mmol), XPhos Pd G2 (257 mg, 0.33 mmol) and cesium carbonate (1.07 g, 3.28 mmol) in dioxane (15 mL) and water (3 mL) was stirred for 4 h at 80° C. under nitrogen atmosphere. Upon cooling to room temperature, the reaction mixture was concentrated under reduced pressure. The resulting residue was purified by silica gel column chromatography, eluted with 10% methanol in dichloromethane to afford a light-yellow solid (670 mg, 91%). LCMS calculated for C₂₄H₂₉N₆O₃ (M+H)⁺ m/z=449.2; found 449.2.

### Step 3: 5-(4-((5-(1,2-Dihydroxyethyl)-3-methyl-2,4-dioxo-1,2,3,4-tetrahydroquinazolin-7-yl)methyl)piperazin-1-yl)-N,6-dimethylpicolinamide

To a mixture of N,6-dimethyl-5-(4-((3-methyl-2,4-dioxo-5-vinyl-1,2,3,4-tetrahydroquinazolin-7-yl)methyl)piperazin-1-yl)picolinamide (600 mg, 1.34 mmol) and 4-methylmorpholine N-oxide (470 mg, 4.0 mmol) in dichloromethane (12 mL) and tert-butanol (1.79 mL) was added potassium osmate (VI) dihydrate (49.3 mg, 0.13 mmol). The resulting mixture was stirred for 16 h at room temperature under nitrogen atmosphere. The reaction was then concentrated under reduced pressure and the residue was purified by silica gel column chromatography, eluted with 8% methanol in dichloromethane to afford a brown solid (550 mg, 76%). LCMS calculated for C₂₄H₃₁N₆O₅ (M+H)⁺ m/z=483.2; found 483.3.

### Step 4: 5-(4-((5-Formyl-3-methyl-2,4-dioxo-1,2,3,4-tetrahydroquinazolin-7-yl)methyl)piperazin-1-yl)-N,6-dimethylpicolinamide

To a mixture of 5-(4-((5-(1,2-dihydroxyethyl)-3-methyl-2,4-dioxo-1,2,3,4-tetrahydroquinazolin-7-yl)methyl)piperazin-1-yl)-N,6-dimethylpicolinamide (500 mg, 1.04 mmol) in methanol (9 mL) and water (9 mL) was added sodium periodate (221 mg, 1.04 mmol) in portions at room temperature. After stirring for 30 min, the reaction was diluted with water (100 mL) and extracted with ethyl acetate (3×100 mL). The combined organic layers were washed with brine (2×100 mL), dried over anhydrous sodium sulfate. After filtration, the filtrate was concentrated under reduced pressure. The resulting residue was purified by silica gel column chromatography, eluted with 10% methanol in dichloromethane to afford a white solid (430 mg, 84%). LCMS calculated for C₂₃H₂₇N₆O₄ (M+H)⁺ m/z=451.2; found 451.2.

### Step 5: 5-(4-((5-(Difluoromethyl)-3-methyl-2,4-dioxo-1,2,3,4-tetrahydroquinazolin-7-yl)methyl)piperazin-1-yl)-N,6-dimethylpicolinamide (I-47)

To a mixture of 5-(4-((5-formyl-3-methyl-2,4-dioxo-1,2,3,4-tetrahydroquinazolin-7-yl)methyl)piperazin-1-yl)-N,6-dimethylpicolinamide (350 mg, 0.78 mmol) in dichloromethane (15 mL) was added diethylaminosulfur trifluoride (1.25 g, 7.8 mmol) dropwise at 0° C. under nitrogen atmosphere. After stirring for 16 h at room temperature, the reaction was quenched with sat. NH₄Cl (aq) and extracted with dichloromethane (3×30 mL). The combined organic layers were washed with brine, dried over anhydrous sodium sulfate. After filtration, the filtrate was concentrated under reduced pressure. The resulting residue was purified by silica gel column chromatography, eluted with 10% methanol in dichloromethane. The product was re-purified by reversed-phase flash chromatography with the following conditions: column, C18 silica gel; mobile phase, acetonitrile in water (0.1% trifluoroacetic acid), 5% to 50% gradient over 30 min; detector, UV 254 nm. The fractions were collected, combined and lyophilized to afford the TFA salt of the desired product as a white solid (349 mg). LCMS calculated for C₂₃H₂₇F₂N₆O₃ (M+H)⁺ m/z=473.2; found 473.3. ¹H NMR (400 MHz, CD₃OD) δ 8.07-7.87 (m, 2H), 7.78 (d, J=1.2 Hz, 1H), 7.57 (d, J=8.4 Hz, 1H), 7.50 (d, J=1.2 Hz, 1H), 4.58 (s, 2H), 3.60-3.48 (m, 4H), 3.39 (s, 3H), 3.29-3.14 (m, 4H), 2.94 (s, 3H), 2.60 (s, 3H).

### Example 48: 5-(4-((5-chloro-3-ethyl-2,4-dioxo-1,2,3,4-tetrahydroquinazolin-7-yl)methyl)piperazin-1-yl)-N-ethyl-6-methylpicolinamide (I-48)

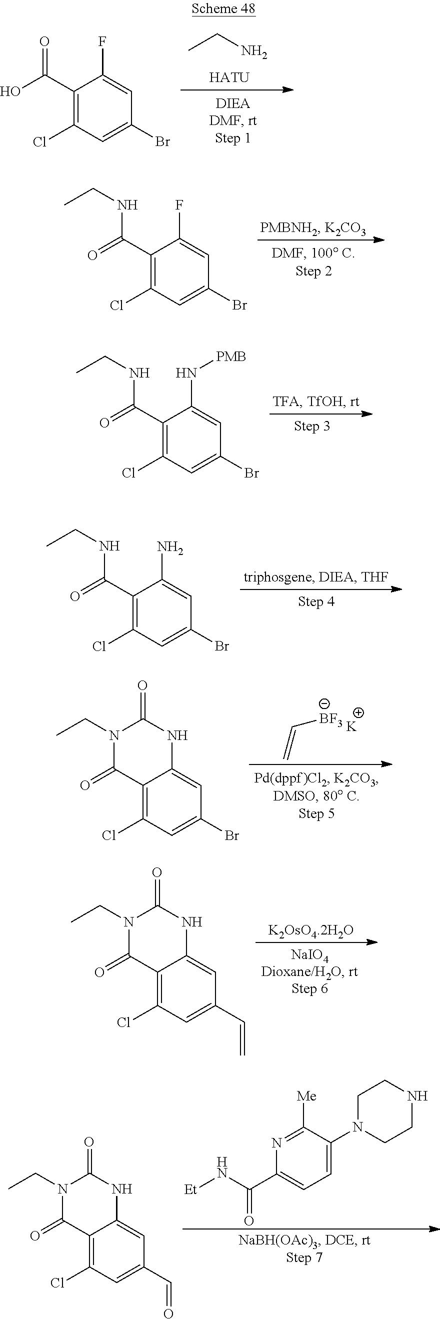

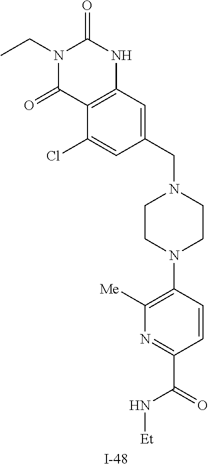

### Step 1: 4-Bromo-2-chloro-N-ethyl-6-fluorobenzamide

A mixture of 4-bromo-2-chloro-6-fluorobenzoic acid (10 g, 39.5 mmol) and 2-(7-azabenzotriazol-1-yl)-N,N,N′,N′-tetramethyluronium hexafluorophosphate (18 g, 47.4 mmol) in N,N-dimethylformamide (100 mL) was stirred for 30 min at room temperature. Ethylamine (2.0 M in THF, 60 mL, 120 mmol) was added, followed by N,N-diisopropylethylamine (15.3 g, 118.4 mmol). The reaction mixture was stirred for 16 h; and then diluted with water (1 L) and extracted with ethyl acetate (3×800 mL). The combined organic layers were washed with brine (2×800 mL), dried over anhydrous sodium sulfate. After filtration, the filtrate was concentrated under reduced pressure and the resulting residue was purified by silica gel column chromatography, eluted with 25% ethyl acetate in petroleum ether to afford a white solid (9.7 g, 88%). LCMS calculated for C₉H9BrClFNO (M+H)⁺ m/z=280.0; found 279.9.

### Step 2: 4-Bromo-2-chloro-N-ethyl-6-((4-methoxybenzyl)amino)benzamide

A mixture of 4-bromo-2-chloro-N-ethyl-6-fluorobenzamide (9.7 g, 36.4 mmol), potassium carbonate (10.06 g, 72.8 mmol) and 4-methoxybenzylamine (6.72 g, 54.6 mmol) in N,N-dimethylformamide (100 mL) was stirred at 100° C. for 16 h. Upon cooling to room temperature, the reaction mixture was diluted with water (1 L) and extracted with ethyl acetate (3×800 mL). The combined organic layers were washed with brine (800 mL), dried over anhydrous sodium sulfate. After filtration, the filtrate was concentrated under reduced pressure. The resulting residue was purified by silica gel column chromatography, eluted with 15% ethyl acetate in petroleum ether to afford a yellow solid (13 g, 90%). LCMS calculated for C₁₇H₁₉BrClN₂O₂ (M+H)⁺ m/z=397.0; found 397.2. ¹H NMR (400 MHz, DMSO-d₆) δ 8.54 (t, J=5.6 Hz, 1H), 7.28-7.20 (m, 2H), 6.93-6.85 (m, 2H), 6.81 (d, J=1.6 Hz, 1H), 6.58 (d, J=1.6 Hz, 1H), 6.03 (t, J=6.0 Hz, 1H), 4.27 (d, J=6.0 Hz, 2H), 3.73 (s, 3H), 3.30-3.24 (m, 1H), 1.15 (t, 7.2 Hz, 3H).

### Step 3:2-Amino-4-bromo-6-chloro-N-ethylbenzamide

To a solution of 4-bromo-2-chloro-N-ethyl-6-((4-methoxybenzyl)amino)benzamide (13 g, 32.7 mmol) in trifluoroacetic acid (30 mL) at 0° C. was added trifluoromethanesulfonic acid (10 mL). The mixture was allowed to warm to room temperature and stirred for 16 h. The reaction was then concentrated under reduced pressure. The residue was diluted with dichloromethane (300 mL) and neutralized to pH=8 with saturated sodium bicarbonate (aq.). The separated aqueous layer was further extracted with dichloromethane (3×300 mL). The combined organic layers were dried over anhydrous sodium sulfate. After filtration, the filtrate was concentrated under reduced pressure. The resulting residue was purified by silica gel column chromatography, eluted with 50% ethyl acetate in petroleum ether to afford as a yellow solid (8.2 g, 89%). LCMS calculated for C₉H₁₁BrClN₂O (M+H)⁺ m/z=277.0; found 277.2. ¹H NMR (300 MHz, CDCl₃) δ 6.88 (d, J=1.2 Hz, 1H), 6.76 (d, J=1.2 Hz, 1H), 3.50 (q, J=7.2 Hz, 2H), 1.26 (t, J=7.2 Hz, 3H).

### Step 4: 7-Bromo-5-chloro-3-ethylquinazoline-2,4(1H,3H)-dione

To a mixture of 2-amino-4-bromo-6-chloro-N-ethylbenzamide (8.2 g, 29.5 mmol) and N,N-diisopropylethylamine (11.4 g, 88.5 mmol) in tetrahydrofuran (100 ml) at 0° C. was added triphosgene (10.5 g, 35.4 mmol) in portions. After stirring for 1 h at room temperature; the reaction was quenched with sat. NH₄Cl (aq.) and extracted with ethyl acetate (3×200 mL). The combined organic layers were dried over anhydrous sodium sulfate. After filtration, the filtrate was concentrated under reduced pressure. The residue was purified by silica gel column chromatography, eluted with 50% ethyl acetate in petroleum ether to afford a yellow solid (7.95 g, 89%). LCMS calculated for C₁₀H₉BrClN₂O₂ (M+H)⁺ m/z=303.0; found 303.0. ¹H NMR (300 MHz, CDCl₃) δ 9.39 (s, 1H), 7.41 (d, J=1.2 Hz, 1H), 7.14 (d, J=1.2 Hz, 1H), 4.11 (q, J=7.2 Hz, 2H), 1.30 (t, J=7.2 Hz, 3H).

### Step 5: 5-Chloro-3-ethyl-7-vinylquinazoline-2,4(1H,3H)-dione

A mixture of 7-bromo-5-chloro-3-ethylquinazoline-2,4(1H,3H)-dione (7.95 g, 26.3 mmol), potassium ethenyltrifluoroboranuide (4.24 g, 31.6 mmol), 1,1′-bis(diphenylphosphino)ferrocene-palladium(II) dichloride dichloromethane complex (2.15 g, 2.6 mmol) and potassium carbonate (10.9 g, 79.1 mmol) in dimethyl sulfoxide (80 mL) was stirred at 80° C. for 16 h under nitrogen atmosphere. Upon cooling to room temperature, the reaction mixture was diluted with water (800 mL) and extracted with ethyl acetate (3×800 mL). The combined organic layers were washed with brine (2×800 mL), dried over anhydrous sodium sulfate. After filtration, the filtrate was concentrated under reduced pressure. The resulting residue was purified by silica gel column chromatography, eluted with 35% ethyl acetate in dichloromethane to afford a white solid (4 g, 60%). LCMS calculated for C₁₂H₁₀ClN₂O₂ (M−H)⁻ m/z=249.1; found 249.0. ¹H NMR (400 MHz, CDCl₃) δ 8.85 (s, 1H), 7.30 (d, J=1.2 Hz, 1H), 6.87 (d, J=1.2 Hz, 1H), 6.66 (dd, J=17.6, 10.8 Hz, 1H), 5.92 (d, J=17.6 Hz, 1H), 5.54 (d, J=10.8 Hz, 1H), 4.11 (q, J=7.2 Hz, 2H), 1.30 (t, J=7.2 Hz, 3H).

### Step 6: 5-Chloro-3-ethyl-2,4-dioxo-1,2,3,4-tetrahydroquinazoline-7-carbaldehyde

A mixture of 5-chloro-3-ethyl-7-vinylquinazoline-2,4(1H,3H)-dione (500 mg, 2.0 mmol) and potassium osmate (VI) dihydrate (147 mg, 0.4 mmol) in dioxane (20 mL) and water (4 mL) was stirred for 30 min at room temperature. Sodium periodate (1.7 g, 8.0 mmol) was then added. The reaction mixture was stirred for additional 2 h, and concentrated under reduced pressure. The resulting residue was purified by silica gel column chromatography, eluted with 40% ethyl acetate in dichloromethane to afford an off-white solid (400 mg, 79%). LCMS calculated for C₁₁H₈ClN₂O₃ (M−H)⁻ m/z=251.0; found 251.0. ¹H NMR (400 MHz, CDCl₃) δ 10.16 (s, 1H), 10.04 (s, 1H), 7.72 (d, J=1.2 Hz, 1H), 7.48 (d, J=1.2 Hz, 1H), 4.16 (q, J=7.2 Hz, 2H), 1.34 (t, J=7.2 Hz, 3H).

### Step 7: 5-(4-((5-Chloro-3-ethyl-2,4-dioxo-1,2,3,4-tetrahydroquinazolin-7-yl)methyl)piperazin-1-yl)-N-ethyl-6-methylpicolinamide (I-48)

To a solution of 5-chloro-3-ethyl-2,4-dioxo-1H-quinazoline-7-carbaldehyde (100 mg, 0.40 mmol) in 1,2-dichloroethane (5 mL) was added N-ethyl-6-methyl-5-(piperazin-1-yl)pyridine-2-carboxamide (108.1 mg, 0.44 mmol). The reaction was stirred at room temperature for 1 h. To the above mixture was added sodium triacetoxyborohydride (109 mg, 0.52 mmol). The reaction was stirred for additional 2 h, and then concentrated under reduced pressure. The residue was purified by Prep-HPLC with the following conditions (Column: Xselect CSH C18 OBD column, 30*150 mm 5 um; mobile Phase A: water (0.05% trifluoroacetate), mobile Phase B: acetonitrile; flow rate: 60 mL/min; Gradient: 8% B to 33% B over 9 min, detector, UV 254/220 nm). Eluted fractions were collected and lyophilized to give the TFA salt of the desired product as a light-yellow solid (156 mg). LCMS calculated for C₂₄H₃₀CIN₆O₃ (M+H)⁺ m/z=485.2; found 485.0; ¹H NMR (300 MHz, CD₃OD) δ 7.91 (d, J=8.4 Hz, 1H), 7.58 (d, J=8.4 Hz, 1H), 7.46 (d, J=1.5 Hz, 1H), 7.27 (d, J=1.5 Hz, 1H), 4.48 (s, 2H), 4.05 (q, J=7.2 Hz, 2H), 3.60-3.49 (m, 4H), 3.43 (q, J=7.2 Hz, 2H), 3.30-3.17 (m, 4H), 2.60 (s, 3H), 1.27-1.18 (m, 6H).

### Example 49: 5-(4-((5-Cyclopropyl-8-fluoro-3-methyl-2,4-dioxo-1,2,3,4-tetrahydroquinazolin-7-yl)methyl)piperazin-1-yl)-N,6-dimethylpicolinamide (I-49)

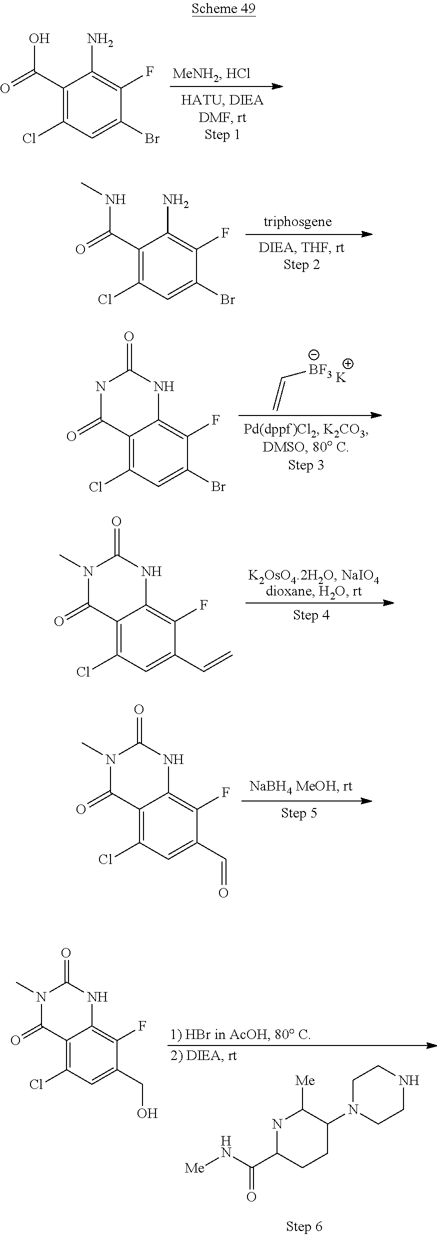

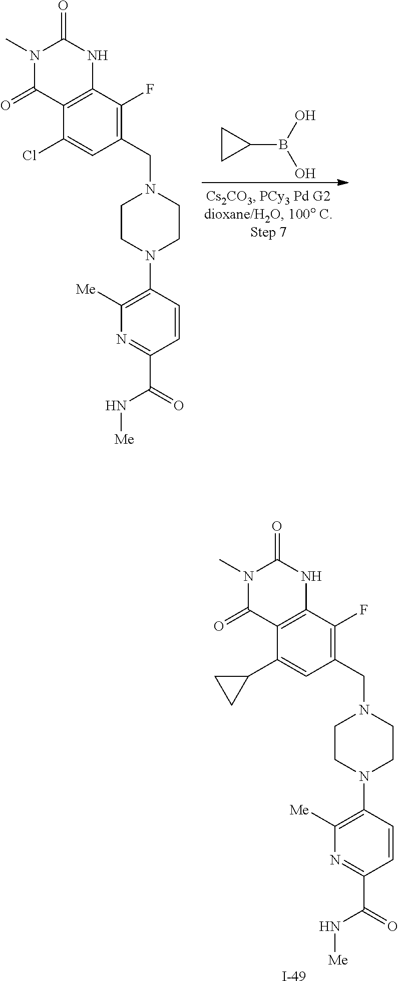

### Step 1:2-Amino-4-bromo-6-chloro-3-fluoro-N-methylbenzamide

To a solution of 2-amino-4-bromo-6-chloro-3-fluorobenzoic acid (Example 39, Step 3:2.2 g, 8.2 mmol) in N,N-dimethylformamide (30 mL) was added 2-(7-azabenzotriazol-1-yl)-N,N,N′,N′-tetramethyluronium hexafluorophosphate (3.74 g, 9.83 mmol). The mixture was stirred at room temperature for 15 min. Methylamine hydrochloride (0.83 g, 12.29 mmol) was added, followed by N,N-diisopropylethylamine (2.12 g, 16.39 mmol). After stirring for additional 16 h, the reaction mixture was diluted with water (300 mL) and extracted with ethyl acetate (3×300 mL). The combined organic layers were washed with brine (2×300 mL), dried over anhydrous Na₂SO₄. After filtration, the filtrate was concentrated under reduced pressure. The resulting residue was purified by silica gel column chromatography, eluted with 50% ethyl acetate in petroleum ether to afford the desired product as a yellow solid (1.5 g, 65%). LCMS calculated for C₈H₈BrClFN₂O (M+H)⁺ m/z=280.9; found 280.9; ¹H NMR (400 MHz, CDCl₃) δ 6.89 (d, J=5.6 Hz, 1H), 6.16 (s, 1H), 4.58 (brs, 2H), 3.02 (d, J=4.8 Hz, 3H).

### Step 2: 7-Bromo-5-chloro-8-fluoro-3-methylquinazoline-2,4(1H,3H)-dione

To a mixture of N,N-diisopropylethylamine (2.85 g, 22.02 mmol) and 2-amino-4-bromo-6-chloro-3-fluoro-N-methylbenzamide (3.1 g, 11.01 mmol) in tetrahydrofuran (60 mL) at 0° C. was added triphosgene (3.27 g, 11.01 mmol) in portions. The reaction was allowed to warm to room temperature and stirred for 1 h. Sat. NH₄Cl (aq) (20 mL) was charged to the reaction mixture. The precipitate was collected by filtration, washed with water (3×20 mL) and ethyl acetate (3×20 mL). The resulting cake was dried under vacuum to give the desired product as a white solid (2.7 g, 80%). LCMS calculated for C₉H₄BrClFN₂O₂ (M−H)⁻ m/z=304.9; found 304.8; ¹H NMR (400 MHz, CDCl₃) δ 8.13 (s, 1H), 7.44 (d, J=6.0 Hz, 1H), 3.44 (s, 3H).

### Step 3: 5-Chloro-8-fluoro-3-methyl-7-vinylquinazoline-2,4(1H,3H)-dione

A mixture of 7-bromo-5-chloro-8-fluoro-3-methyl-1H-quinazoline-2,4-dione (5 g, 16.26 mmol), potassium ethenyltrifluoroboranuide (4.36 g, 32.52 mmol), potassium carbonate (6.74 g, 48.78 mmol) and 1,1′-bis(diphenylphosphino)ferrocene-palladium(II) dichloride dichloromethane complex (2.65 g, 3.25 mmol) in dimethyl sulfoxide (50 mL) was stirred at 80° C. for 1 h under nitrogen atmosphere. Upon cooling to room temperature, the reaction was diluted with water (500 mL) and extracted with ethyl acetate (5×500 mL). The combined organic layers were washed with brine (2×400 mL), dried over anhydrous sodium sulfate. After filtration, the filtrate was concentrated under reduced pressure. The resulting residue was purified by silica gel column chromatography, eluted with 20% ethyl acetate in dichloromethane to give the desired product as a brown solid (3 g, 72%). LCMS calculated for C₁₁H₇ClFN₂O₂ (M−H)⁻ m/z=253.0; found 253.0.

### Step 4: 5-Chloro-8-fluoro-3-methyl-2,4-dioxo-1,2,3,4-tetrahydroquinazoline-7-carbaldehyde

A mixture of 5-chloro-7-ethenyl-8-fluoro-3-methyl-1H-quinazoline-2,4-dione (2.9 g, 11.39 mmol) and potassium osmate (VI) dihydrate (0.84 g, 2.28 mmol) in 1,4-dioxane (30 mL) and water (10 mL) was stirred for 30 min at room temperature. Sodium periodate (9.74 g, 45.55 mmol) was charged. The reaction mixture was stirred for additional 2 h, and then concentrated under reduced pressure. The resulting residue was purified by silica gel column chromatography, eluted with 50% ethyl acetate in dichloromethane to give the desired product as an orange solid (1.8 g, 62%). LCMS calculated for C₁₀H₅ClFN₂O₃ (M−H)⁻ m/z=255.0; found 254.9.

### Step 5: 5-Chloro-8-fluoro-7-(hydroxymethyl)-3-methylquinazoline-2,4(1H,3H)-dione

A mixture of 5-chloro-8-fluoro-3-methyl-2,4-dioxo-1H-quinazoline-7-carbaldehyde (400 mg, 1.6 mmol) in methanol (10 mL) was treated with sodium borohydride (118 mg, 3.12 mmol) in portions at 0° C. The resulting mixture was stirred at room temperature for 1 h; and then quenched with water (5 mL). The resulting mixture was concentrated under reduced pressure. The resulting residue was purified by silica gel column chromatography, eluted with 10% methanol in dichloromethane to provide the desired product as a yellow solid (325 mg, 81%). LCMS calculated for C₁₀H₇ClFN₂O₃ (M−H)⁻ m/z=257.0; found 256.8.

### Step 6: 5-(4-((5-Chloro-8-fluoro-3-methyl-2,4-dioxo-1,2,3,4-tetrahydroquinazolin-7-yl)methyl)piperazin-1-yl)-N,6-dimethylpicolinamide

To 5-chloro-8-fluoro-7-(hydroxymethyl)-3-methyl-1H-quinazoline-2,4-dione (300 mg, 1.16 mmol) in was added hydrogen bromide (33 wt. % solution in glacial acid, 5 mL). The reaction was heated at 80° C. under nitrogen atmosphere for 2 h. Upon cooling to room temperature, the reaction was concentrated under reduced pressure. To the residue was added acetonitrile (5 mL), followed by N,6-dimethyl-5-(piperazin-1-yl)picolinamide (236 mg, 1.1 mmol) and N-ethyl-N-isopropylpropan-2-amine (1.3 g, 11.6 mmol). The resulting mixture was stirred at room temperature for 16 h, and then concentrated under reduced pressure. The resulting residue was purified by silica gel column chromatography, eluted with 7% methanol in dichloromethane to provide the desired product as a yellow solid (240 mg, 50%). LCMS calculated for C₂₂H₂₅ClFN₆O₃ (M+H)⁺ m/z=475.2; found 475.2. ¹H NMR (400 MHz, CD₃OD) δ 7.91 (d, J=8.4 Hz, 1H), 7.55 (d, J=8.4 Hz, 1H), 7.02 (d, J=6.4 Hz, 1H), 4.56 (s, 2H), 3.63-3.50 (m, 6H), 3.37 (s, 3H), 3.25-3.16 (m, 2H), 2.95 (s, 3H), 2.60 (s, 3H).

### Step 7: 5-(4-((5-Cyclopropyl-8-fluoro-3-methyl-2,4-dioxo-1,2,3,4-tetrahydroquinazolin-7-yl)methyl)piperazin-1-yl)-N,6-dimethylpicolinamide (I-49)

A mixture of 5-(4-((5-chloro-8-fluoro-3-methyl-2,4-dioxo-1,2,3,4-tetrahydroquinazolin-7-yl)methyl)piperazin-1-yl)-N,6-dimethylpicolinamide (100 mg, 0.21 mmol), cyclopropylboronic acid (54 mg, 0.63 mmol), cesium carbonate (206 mg, 0.63 mmol) and PCy₃ Pd G2 (50 mg, 0.08 mmol) in dioxane (3 mL) and water (0.3 mL) was stirred at 100° C. for 16 h under nitrogen atmosphere. Upon cooling to room temperature, the reaction mixture was concentrated under reduced pressure. The resulting residue was purified by silica gel column chromatography and eluted with 10% methanol in dichloromethane to provide the desired product. The product was re-purified by reversed-phase flash chromatography with the following conditions: column, C18 silica gel; mobile phase, acetonitrile in water (0.1% trifluoroacetic acid), 10% to 50% gradient in 25 min, detector, UV 254 nm. The fractions were collected, combined and lyophilized to provide the TFA salt of the desired product as a white solid (56 mg). LCMS calculated for C₂₅H₃₀FN₆O₃ (M+H)⁺ m/z=481.2; found 481.2. ¹H NMR (400 MHz, CD₃OD) δ 7.90 (d, J=8.4 Hz, 1H), 7.56 (d, J=8.4 Hz, 1H), 7.02 (d, J=6.4 Hz, 1H), 4.55 (s, 2H), 3.65-3.52 (m, 6H), 3.39 (s, 3H), 3.27-3.17 (m, 3H), 2.94 (s, 3H), 2.59 (s, 3H), 1.12-1.06 (m, 2H), 0.82-0.76 (m, 2H).

### Example 50: 5-(4-((3-Ethyl-9-fluoro-5-methoxy-2-oxo-2,3-dihydro-1H-pyrimido[4,5,6-de]quinazolin-8-yl)methyl)piperazin-1-yl)-N,6-dimethylpicolinamide (I-50)

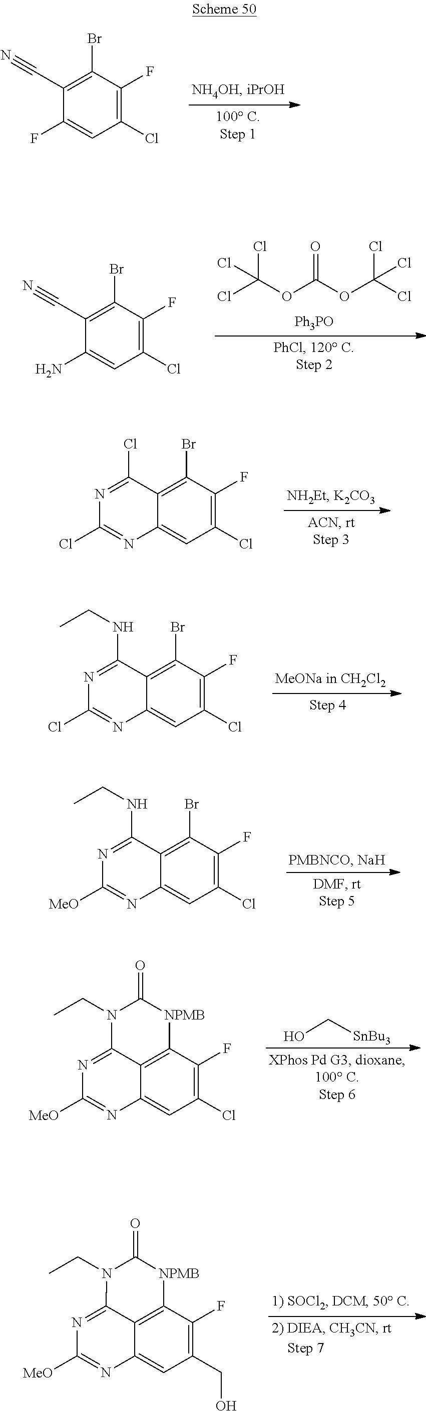

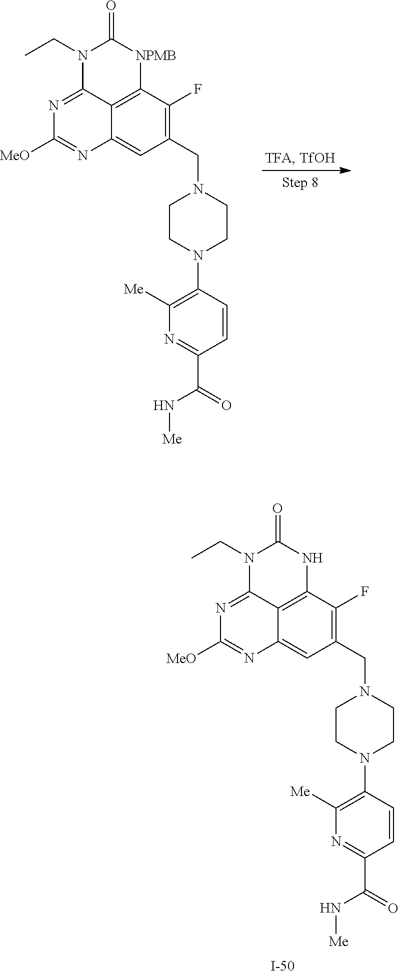

### Step 1: 6-Amino-2-bromo-4-chloro-3-fluorobenzonitrile

To a solution of 2-bromo-4-chloro-3,6-difluorobenzonitrile (Example 34, Step 1: 2.0 g, 7.92 mmol) in isopropanol (4 mL) was added a NH₄OH solution (28% NH₃ in H₂O, 20 mL). The mixture was stirred at 100° C. for 16 h. Upon cooling to room temperature, the reaction mixture was extracted with ethyl acetate (3×50 mL). The combined organic layers were dried over anhydrous sodium sulfate and filtered. The filtrate was concentrated under vacuum. The residue was purified by silica gel column chromatography, eluted with 20% ethyl acetate in petroleum ether to afford the desired product as a white solid (1.1 g, 56%). LCMS calculated for CH₄BrClFN₂ (M+H)⁺ m/z=248.9; found 248.9; ¹H NMR (300 MHz, CDCl₃) δ 6.78 (d, J=5.4 Hz, 1H), 4.50 (brs, 2H).

### Step 2: 5-Bromo-2,4,7-trichloro-6-fluoroquinazoline

To a mixture of triphenylphosphine oxide (4.46 g, 16.03 mmol) in chlorobenzene (40 mL) at 0° C. was added triethylamine (0.2 mL, 1.44 mmol), followed by triphosgene (2.38 g, 8.02 mmol). The reaction mixture was stirred at room temperature for 30 min. Then 6-amino-2-bromo-4-chloro-3-fluorobenzonitrile (2 g, 8.02 mmol) was added. The mixture was stirred at 120° C. for 2 h. Upon cooling to room temperature, the mixture was diluted with water (80 mL) and extracted with dichloromethane (3×100 mL). The combined organic layers were dried over anhydrous sodium sulfate. After filtration, the filtrate was concentrated under reduced pressure. The residue was purified by silica gel column chromatography, eluted with 10% ethyl acetate in petroleum ether to afford the desired product as a white solid (1.7 g, 64%). ¹H NMR (300 MHz, CDCl₃) δ 8.11 (d, J=6.9 Hz, 1H).

### Step 3: 5-Bromo-2,7-dichloro-N-ethyl-6-fluoroquinazolin-4-amine

To a mixture of 5-bromo-2,4,7-trichloro-6-fluoroquinazoline (2.2 g, 6.66 mmol) and potassium carbonate (2.76 g, 19.98 mmol) in acetonitrile (22 mL) was added ethylamine (2 M in tetrahydrofuran, 5 mL, 10 mmol) dropwise at 0° C. After stirring at room temperature for 2 h, the reaction mixture was concentrated under vacuum. The residue was purified by silica gel column chromatography, eluted with 20% ethyl acetate in petroleum ether to afford the desired product as a yellow solid (2.1 g, 93%). LCMS calculated for C₁₀H₈BrCl₂FN₃ (M+H)⁺ m/z=337.9; found 337.9.

### Step 4: 5-Bromo-7-chloro-N-ethyl-6-fluoro-2-methoxyquinazolin-4-amine

The mixture of 5-bromo-2,7-dichloro-N-ethyl-6-fluoroquinazolin-4-amine (1.2 g, 3.54 mmol), sodium methanolate (382.48 mg, 7.08 mmol) in dichloromethane (12 mL) was stirred for 16 h at room temperature. Water (10 mL) was then added. The mixture was extracted with ethyl acetate (3×20 mL). The combined organic layers were dried over anhydrous sodium sulfate. After filtration, the filtrate was concentrated under reduced pressure. The residue was purified by silica gel column chromatography, eluted with 30% ethyl acetate in petroleum ether to afford the desired product as a white solid (513 mg, 43%). LCMS calculated for C₁₁H₁₁BrClFN₃O (M+H)⁺ m/z=334.0; found 333.9; ¹H NMR (400 MHz, CDCl₃) δ 7.85 (brs, 1H), 7.73 (d, J=6.8 Hz, 1H), 4.01 (s, 3H), 3.71-3.64 (m, 2H), 1.37 (t, J=7.2 Hz, 3H).

### Step 5: 8-Chloro-3-ethyl-9-fluoro-5-methoxy-1-(4-methoxybenzyl)-1H-pyrimido[4,5,6-de]quinazolin-2(3H)-one

To a solution of 5-bromo-7-chloro-N-ethyl-6-fluoro-2-methoxyquinazolin-4-amine (200 mg, 0.6 mmol) in N,N-dimethylformamide (2 ml) at 0° C. under nitrogen atmosphere was added sodium hydride (60% dispersion in mineral oil, 48 mg, 1.2 mmol). After stirring at 0° C. for 30 min, the reaction mixture was treated with 1-(isocyanatomethyl)-4-methoxybenzene (146 mg, 0.9 mmol). The resulting mixture was allowed to warm to room temperature and stirred for additional 16 h. The mixture was then diluted with dichloromethane (20 mL), washed with brine (3×10 mL). The separated organic layer was dried over anhydrous sodium sulfate. After filtration, the filtrate was concentrated under reduced pressure. The residue was purified by silica gel column chromatography, eluted with 50% ethyl acetate in petroleum ether to afford the desired product as a white solid (130 mg, 52%). LCMS calculated for C₂₀H₁₉ClFN₄O₃ (M+H)⁺ m/z=417.1; found 417.1.

### Step 6:3-Ethyl-9-fluoro-8-(hydroxymethyl)-5-methoxy-1-(4-methoxybenzyl)-1H-pyrimido[4,5,6-de]quinazolin-2(3H)-one

The mixture of 8-chloro-3-ethyl-9-fluoro-5-methoxy-1-(4-methoxybenzyl)-1H-pyrimido[4,5,6-de]quinazolin-2(3H)-one (110 mg, 0.26 mmol), methanesulfonato (2-dicyclohexylphosphino-2′,4′,6′-tri-i-propyl-1,1′-biphenyl)(2′-amino-1,1′-biphenyl-2-yl)palladium(II) (45 mg, 0.05 mmol) and (tributylstannyl)methanol (169 mg, 0.52 mmol) in dioxane (2 mL) was stirred at 100° C. for 2 h under nitrogen atmosphere. Upon cooling to room temperature, the reaction mixture was concentrated under reduced pressure. The residue was purified by silica gel column chromatography, eluted with 13% methanol in dichloromethane to afford the desired product as a white solid (77 mg, 71%). LCMS calculated for C₂₁H₂₂FN₄O₄ (M+H)⁺ m/z=413.2; found 413.2.

### Step 7: 5-(4-((3-Ethyl-9-fluoro-5-methoxy-1-(4-methoxybenzyl)-2-oxo-2,3-dihydro-1H-pyrimido[4,5,6-de]quinazolin-8-yl)methyl)piperazin-1-yl)-N,6-dimethylpicolinamide

To a solution of 3-ethyl-9-fluoro-8-(hydroxymethyl)-5-methoxy-1-(4-methoxybenzyl)-1H-pyrimido[4,5,6-de]quinazolin-2(3H)-one (55 mg, 0.13 mmol) in dichloromethane (2 mL) was added sulfurous dichloride (158.64 mg, 1.33 mmol), followed by one drop of N,N-dimethylformamide. The mixture was stirred at 50° C. for 2 h under nitrogen atmosphere. Up cooling to room temperature, the reaction mixture was concentrated under vacuum. To the residue was added acetonitrile (3 mL), followed by N,6-dimethyl-5-(piperazin-1-yl)pyridine-2-carboxamide (41 mg, 0.17 mmol) and N-ethyl-N-isopropylpropan-2-amine (172 mg, 1.33 mmol). The resulting mixture was stirred at room temperature for 16 h; and then concentrated under vacuum. The residue was purified by Prep-TLC (5% methanol in dichloromethane) to afford the desired product as a white solid (50 mg, 60%). LCMS calculated for C₃₃H₃₈FN₈O₄ (M+H)⁺ m/z=629.3; found 629.4.

### Step 8: 5-(4-((3-ethyl-9-fluoro-5-methoxy-2-oxo-2,3-dihydro-1H-pyrimido[4,5,6-de]quinazolin-8-yl)methyl)piperazin-1-yl)-N,6-dimethylpicolinamide (I-50)

To a solution of 5-(4-((3-ethyl-9-fluoro-5-methoxy-1-(4-methoxybenzyl)-2-oxo-2,3-dihydro-1H-pyrimido[4,5,6-de]quinazolin-8-yl)methyl)piperazin-1-yl)-N,6-dimethylpicolinamide (50 mg, 0.08 mmol) in 2,2,2-trifluoroacetic acid (1 mL) was added trifluoromethanesulfonic acid (0.2 mL) at room temperature. After stirring at room temperature for 16 h, the mixture was concentrated under vacuum. The residue was purified by reversed-phase flash chromatography with the following conditions: column, C18 silica gel; mobile phase, acetonitrile in water (0.1% trifluoroacetic acid), 10% to 50% gradient over 10 min; detector, UV 254 nm. The collected fractions were lyophilized to afford the TFA salt of the desired product as a white solid (30 mg). LCMS calculated for C₂₅H₃₀FN₈O₃ (M+H)⁺ m/z=509.2; found 509.2; ¹H NMR (400 MHz, DMSO-d₆) δ 11.83 (s, 1H), 10.11 (s, 1H), 8.47 (q, J=4.8 Hz, 1H), 7.83 (d, J=8.0 Hz, 1H), 7.56 (d, J=8.4 Hz, 1H), 7.39 (d, J=5.2 Hz, 1H), 4.61 (s, 2H), 4.10 (q, J=7.2 Hz, 2H), 3.95 (s, 3H), 3.56-3.27 (m, 6H), 3.12-2.99 (m, 2H), 2.81 (d, J=4.8 Hz, 3H), 2.52 (s, 3H), 1.21 (t, J=7.2 Hz, 3H).

### Example 51: 5-(4-((3-Ethyl-9-fluoro-6-methyl-2,5-dioxo-2,3,5,6-tetrahydro-1H-pyrimido[4,5,6-de]quinazolin-8-yl)methyl)piperazin-1-yl)-N,6-dimethylpicolinamide (I-51)

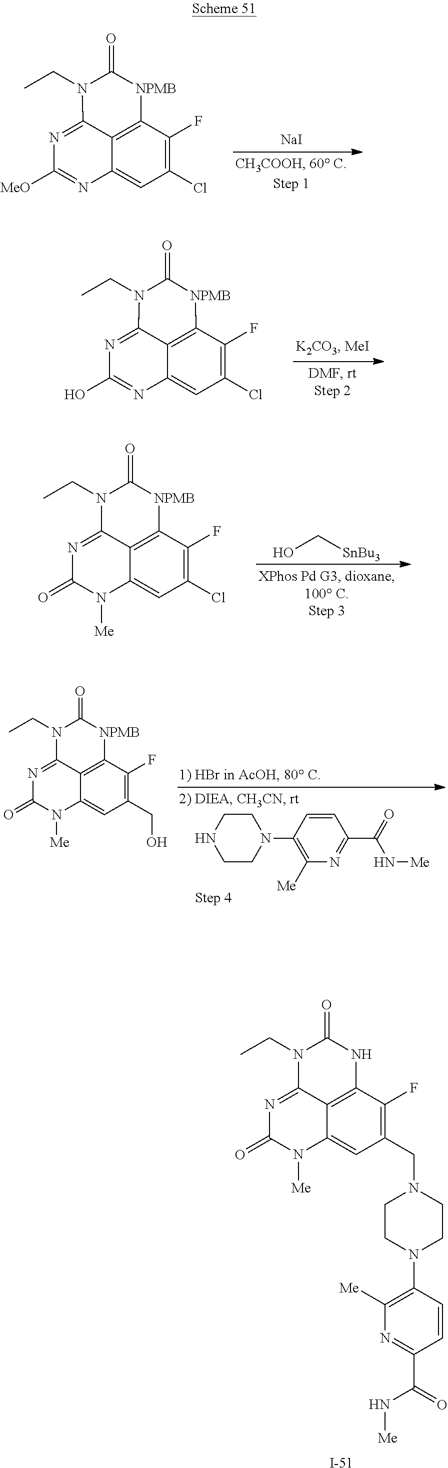

### Step 1: 8-Chloro-3-ethyl-9-fluoro-5-hydroxy-1-(4-methoxybenzyl)-1H-pyrimido[4,5,6-de]quinazolin-2(3H)-one

The mixture of 8-chloro-3-ethyl-9-fluoro-5-methoxy-1-(4-methoxybenzyl)-1H-pyrimido[4,5,6-de]quinazolin-2(3H)-one (Example 50, Step 5:130 mg, 0.31 mmol) and sodium iodide (234 mg, 1.56 mmol) in acetic acid (3 mL) was stirred for 1 h at 60° C. Upon cooling to room temperature, the reaction mixture was treated with a saturated aqueous sodium sulfite solution (5 mL) followed by a saturated aqueous sodium bicarbonate solution (20 mL). The mixture was extracted with ethyl acetate (3×20 mL). The combined organic layers were washed with brine (30 mL), dried over anhydrous sodium sulfate. After filtration, the filtrate was concentrated under reduced pressure. The residue was purified by silica gel column chromatography, eluted with 10% methanol in dichloromethane to afford the desired product as a white solid (100 mg, 80%). LCMS calculated for C₁₉H₁₇ClFN₄O₃ (M+H)⁺ m/z=403.1; found 403.1.

### Step 2: 8-Chloro-3-ethyl-9-fluoro-1-(4-methoxybenzyl)-6-methyl-1H-pyrimido[4,5,6-de]quinazoline-2,5 (3H,6H)-dione

To a mixture of 8-chloro-3-ethyl-9-fluoro-5-hydroxy-1-(4-methoxybenzyl)-1H-pyrimido[4,5,6-de]quinazolin-2(3H)-one (110 mg, 0.27 mmol) and potassium carbonate (45 mg, 0.33 mmol) in N,N-dimethylformamide (3 mL) was added iodomethane (97 mg, 0.68 mmol) at 0° C. The reaction mixture was stirred at room temperature for 3 h, and then diluted with ethyl acetate (20 mL) and washed with brine (3×10 mL). The separated organic layer was dried over sodium sulfate and filtered. The filtrate was concentrated under vacuum. The residue was purified by silica gel column chromatography, eluted with 50% ethyl acetate in dichloromethane to afford the desired product as a white solid (98 mg, 86%). LCMS calculated for C₂₀H₁₉ClFN₄O₃ (M+H)⁺ m/z=417.1; found 417.1; ¹H NMR (300 MHz, CDCl₃) δ 7.27-7.24 (m, 2H), 6.91-6.86 (m, 3H), 5.50 (d, J=2.4 Hz, 2H), 4.41 (q, J=7.2 Hz, 2H), 3.80 (s, 3H), 3.59 (s, 3H), 1.36 (t, J=7.2 Hz, 3H).

### Step 3:3-Ethyl-9-fluoro-8-(hydroxymethyl)-1-(4-methoxybenzyl)-6-methyl-1H-pyrimido[4,5,6-de]quinazoline-2,5 (3H,6H)-dione

The mixture of 8-chloro-3-ethyl-9-fluoro-1-(4-methoxybenzyl)-6-methyl-1H-pyrimido[4,5,6-de]quinazoline-2,5 (3H,6H)-dione (60 mg, 0.14 mmol), (tributylstannyl)methanol (92 mg, 0.29 mmol) and methanesulfonato (2-dicyclohexylphosphino-2′,4′,6′-tri-i-propyl-1,1′-biphenyl)(2′-amino-1,1′-biphenyl-2-yl)palladium(II) (24 mg, 0.03 mmol) in dioxane (3 mL) was stirred at 100° C. for 2 h under nitrogen atmosphere. Upon cooling to room temperature, the reaction mixture was concentrated under reduced pressure. The residue was purified by silica gel column chromatography, eluted with 13% methanol in dichloromethane to afford the desired product as a white solid (55 mg, 93%). LCMS calculated for C₂₁H₂₂FN₄O₄ (M+H)⁺ m/z=413.2; found 413.3; ¹H NMR (300 MHz, CDCl₃) δ 7.25-7.23 (m, 2H), 7.05-7.02 (m, 1H), 6.88-6.86 (m, 2H), 5.49 (s, 2H), 4.89 (s, 2H), 4.20 (q, J=7.2 Hz, 2H), 3.80 (s, 3H), 3.65 (s, 3H), 1.37 (t, J=7.2 Hz, 3H).

### Step 4: 5-(4-((3-Ethyl-9-fluoro-6-methyl-2,5-dioxo-2,3,5,6-tetrahydro-1H-pyrimido[4,5,6-de]quinazolin-8-yl)methyl)piperazin-1-yl)-N,6-dimethylpicolinamide (I-51)

To 3-Ethyl-9-fluoro-8-(hydroxymethyl)-1-(4-methoxybenzyl)-6-methyl-1H-pyrimido[4,5,6-de]quinazoline-2,5 (3H,6H)-dione (60 mg, 0.15 mmol) was added hydrogen bromide (33 wt. % solution in glacial acid, 2 mL) at room temperature. The reaction mixture was stirred at 80° C. under nitrogen atmosphere for 2 h. Upon cooling to room temperature, the reaction mixture was concentrated under reduced pressure. To the residue was added acetonitrile (3 mL), followed by N,6-dimethyl-5-(piperazin-1-yl)picolinamide (44 mg, 0.19 mmol) and N-ethyl-N-isopropylpropan-2-amine (94 mg, 0.73 mmol). The resulting mixture was stirred at room temperature for 16 h, and then concentrated under vacuum. The residue was purified by Prep-HPLC (column: XBridge Prep Phenyl OBD Column 19*250 mm, 5 um; mobile phase A: water (0.05% trifluoroacetic acid), mobile phase B: acetonitrile; flow rate: 60 mL/min; gradient: 7% B to 22% B over 8 min; detector: UV 254/220 nm). The collected fractions were lyophilized to provide the TFA salt of the desired product as a white solid (35 mg). LCMS calculated for C₂₅H₃₀FN₈O₃ (M+H)⁺ m/z=509.2; found 509.2; ¹H NMR (300 MHz, CD₃OD) δ 7.93 (d, J=8.4 Hz, 1H), 7.61 (d, J=8.4 Hz, 1H), 7.21-7.18 (m, 1H), 4.70 (s, 2H), 4.27 (q, J=6.9 Hz, 2H), 3.70-3.65 (m, 4H), 3.64 (s, 3H), 3.41-3.35 (m, 4H), 2.96 (s, 3H), 2.63 (s, 3H), 1.32 (t, J=6.9 Hz, 3H).

### Example 52: 5-(4-((3-Ethyl-9-fluoro-5-methyl-2-oxo-2,3-dihydro-1H-pyrimido[4,5,6-de]quinazolin-8-yl)methyl)piperazin-1-yl)-N,6-dimethylpicolinamide (I-52)

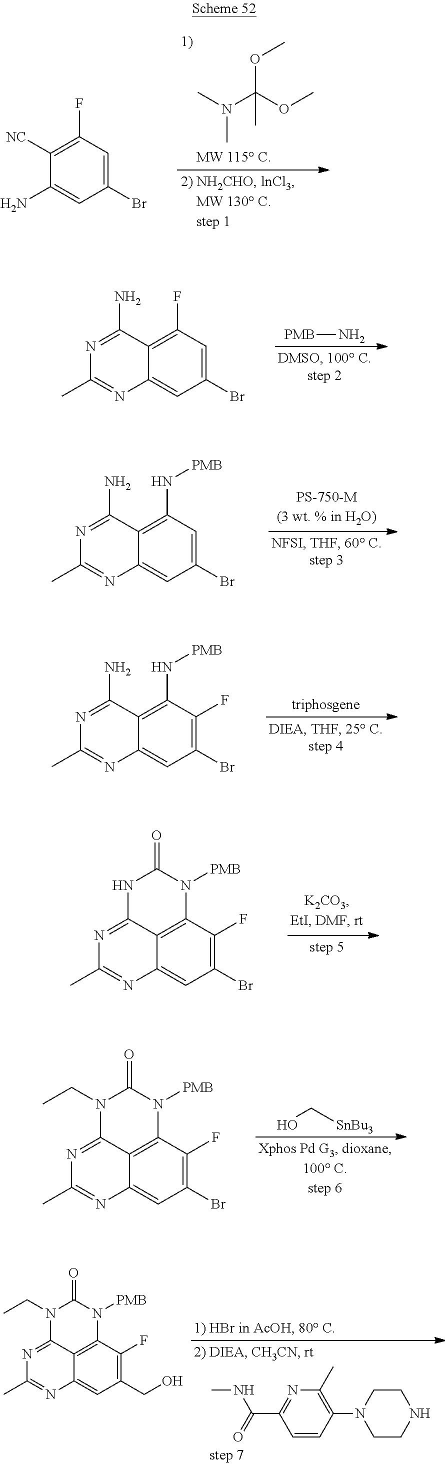

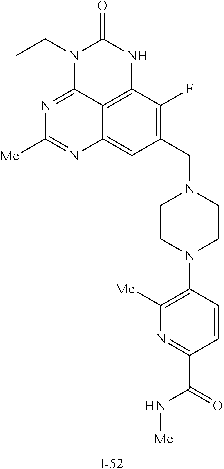

### Step 1: 7-Bromo-5-fluoro-2-methylquinazolin-4-amine

A vail containing a suspension of 2-amino-4-bromo-6-fluorobenzonitrile (3.1 g, 14.42 mmol) in (1,1-dimethoxyethyl) dimethylamine (4.8 g, 36.05 mmol) was sealed and subjected to microwave irradiation (400 W) at 115° C. for 2 min. The mixture was cooled to room temperature and concentrated. To the residue was added formamide (30 mL), followed by indium chloride (3.19 g, 14.42 mmol). The resulting mixture was subjected to microwave irradiation (400W) at 130° C. for 1 h. After cooling to room temperature, the mixture was diluted with water (150 mL) and extracted with ethyl acetate (3×100 mL). The combined organic phases were dried over anhydrous sodium sulfate, filtered and concentrated. The residue was purified by silica gel column chromatography, eluted with 5% methanol in dichloromethane to give the desired product as a white solid (2 g, 54%). LCMS calculated for C₉H₈BrFN₃ (M+H)⁺ m/z=256.0; found 256.0.

### Step 2: 7-Bromo-N⁵-(4-methoxybenzyl)-2-methylquinazoline-4,5-diamine

To a solution of 7-bromo-5-fluoro-2-methylquinazolin-4-amine (1 g, 3.91 mmol) in dimethyl sulfoxide (20 mL) was added (4-methoxyphenyl) methanamine (1.61 g, 11.72 mmol). The reaction was stirred at 100° C. for 16 h. After cooling to room temperature, the mixture was diluted with water (100 mL), and extracted with ethyl acetate (3×100 mL). The combined organic layer was dried over anhydrous sodium sulfate. After filtration, the filtrate was concentrated under reduced pressure. The residue was purified by silica gel column chromatography, eluted with 50% ethyl acetate in petroleum ether to give the desired product as a yellow solid (500 mg, 34%). LCMS calculated for C₁₇H₁₈BrN₄O (M+H)⁺ m/z=373.1; found 373.2.

### Step 3: 7-Bromo-6-fluoro-N°-(4-methoxybenzyl)-2-methylquinazoline-4,5-diamine

To a screw-cap vial equipped with a magnetic stir bar was added 7-bromo-N⁵-(4-methoxybenzyl)-2-methylquinazoline-4,5-diamine (200 mg, 0.54 mmol) and PS-750-M (3 wt % in water, 0.3 mL). The vial was sealed with a Teflon-lined septum, evacuated and backfilled with nitrogen (this process was repeated a total of three times). A solution of N-fluorobenzenesulfonimide (211 mg, 0.67 mmol) in anhydrous tetrahydrofuran (0.2 mL) was added. The resulting mixture was stirred at 60° C. for 16 h. After cooling to room temperature, the reaction was concentrated. The residue was purified by silica gel column chromatography, eluted with 10% methanol in dichloromethane to give the desired product as a yellow solid (100 mg, 48%). LCMS calculated for C₁₇H₁₇BrFN₄O (M+H)⁺ m/z=391.1; found 390.9.

### Step 4: 8-Bromo-9-fluoro-1-(4-methoxybenzyl)-5-methyl-1H-pyrimido[4,5,6-de]quinazolin-2(3H)-one

To a stirred solution of 7-bromo-6-fluoro-N⁵-(4-methoxybenzyl)-2-methylquinazoline-4,5-diamine (400 mg, 1.022 mmol) and N-ethyl-N-isopropylpropan-2-amine (264 mg, 2.04 mmol) in tetrahydrofuran (7 mL) was added triphosgene (303 mg, 1.02 mmol) in portions at 0° C. The reaction was stirred at room temperature for 1 h. The resulting mixture was concentrated. The residue was purified by silica gel column chromatography, eluted with 50% ethyl acetate in petroleum ether to give the desired product as a white solid (400 mg, 50%). LCMS calculated for C₁₈H₁₅BrFN₄O₂ (M+H)⁺ m/z=417.0; found 417.0.

### Step 5: 8-Bromo-3-ethyl-9-fluoro-1-(4-methoxybenzyl)-5-methyl-1H-pyrimido[4,5,6-de]quinazolin-2(3H)-one

To a stirred mixture of 8-bromo-9-fluoro-1-(4-methoxybenzyl)-5-methyl-1H-pyrimido[4,5,6-de]quinazolin-2(3H)-one (350 mg, 0.84 mmol) and potassium carbonate (232 mg, 1.68 mmol) in N,N-dimethylformamide (10 mL) was added iodoethane (196 mg, 1.26 mmol) dropwise at 0° C. After stirring at room temperature for 3 h, the mixture was diluted with water (30 mL) and extracted with ethyl acetate (3×50 mL). The combined organic layer was dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (0.1% formic acid), 30% to 80% gradient over 20 min; detector, UV 254 nm. The fractions were collected and concentrated to give the desired product as a light-yellow solid (200 mg, 54%). LCMS calculated for C₂₀H₁₉BrFN₄O₂ (M+H)⁺ m/z=445.1; found 445.1.

### Step 6:3-Ethyl-9-fluoro-8-(hydroxymethyl)-1-(4-methoxybenzyl)-5-methyl-1H-pyrimido[4,5,6-de]quinazolin-2(3H)-one

To a screw-cap vial equipped with a magnetic stir bar was added 8-bromo-3-ethyl-9-fluoro-1-(4-methoxybenzyl)-5-methyl-1H-pyrimido[4,5,6-de]quinazolin-2 (3H)-one (200 mg, 0.45 mmol), XPhos Pd G3 (76 mg, 0.09 mmol) and (tributylstannyl)methanol (173 mg, 0.54 mmol). The vial was sealed with a Teflon-lined septum, evacuated and backfilled with nitrogen (this process was repeated a total of three times). 1,4-Dioxane (3 mL) was added. The reaction was stirred at 100° C. for 3 h. After cooling to room temperature, the mixture was concentrated. The residue was purified by silica gel column chromatography, eluted with 10% methanol in dichloromethane to give the desired product as a light-yellow solid (130 mg, 73%). LCMS calculated for C₂₁H₂₂FN₄O₃ (M+H)⁺ m/z=397.2; found 397.0.

### Step 7: 5-(4-((3-Ethyl-9-fluoro-5-methyl-2-oxo-2,3-dihydro-1H-pyrimido[4,5,6-de]quinazolin-8-yl)methyl)piperazin-1-yl)-N,6-dimethylpicolinamide (I-52)

To 3-ethyl-9-fluoro-8-(hydroxymethyl)-1-(4-methoxybenzyl)-5-methyl-1H-pyrimido[4,5,6-de]quinazolin-2(3H)-one (150 mg, 0.38 mmol) was added hydrogen bromide (33 wt. % solution in glacial acid, 4 mL) at room temperature. The reaction mixture was heated at 80° C. under nitrogen atmosphere for 2 h. Upon cooling to room temperature, the reaction mixture was concentrated under reduced pressure. To the residue was added acetonitrile (5 mL), followed by N,6-dimethyl-5-(piperazin-1-yl)picolinamide (106 mg, 0.45 mmol) and N-ethyl-N-isopropylpropan-2-amine (488 mg, 3.79 mmol). The resulting mixture was stirred at room temperature for 16 h, and then was concentrated under vacuum. The residue was purified by reversed-phase flash chromatography (C18 silica gel; mobile phase, acetonitrile in water (0.1% trifluoroacetic acid), 10% to 50% gradient over 10 min; detector, UV 254 nm. The collected fractions were lyophilized to provide the TFA salt of the desired product as a white solid (67 mg). LCMS calculated for C₂₅H₃₀FN₈O₂ (M+H)⁺ m/z=493.2; found 493.2; ¹H NMR (300 MHz, CD₃OD) δ 7.92 (d, J=8.1 Hz, 1H), 7.60-7.52 (m, 2H), 4.70 (s, 2H), 4.40 (q, J=6.6 Hz, 2H), 3.62-3.50 (m, 4H), 3.31-3.26 (m, 4H), 2.97 (s, 3H), 2.80 (s, 3H), 2.62 (s, 3H), 1.38 (t, J=6.6 Hz, 3H).

### Example 53: 5-(4-((3-Ethyl-9-fluoro-2-oxo-2,3-dihydro-1H-pyrimido[4,5,6-de]quinazolin-8-yl)methyl-d2)piperazin-1-yl)-N,6-dimethylpicolinamide (I-53)

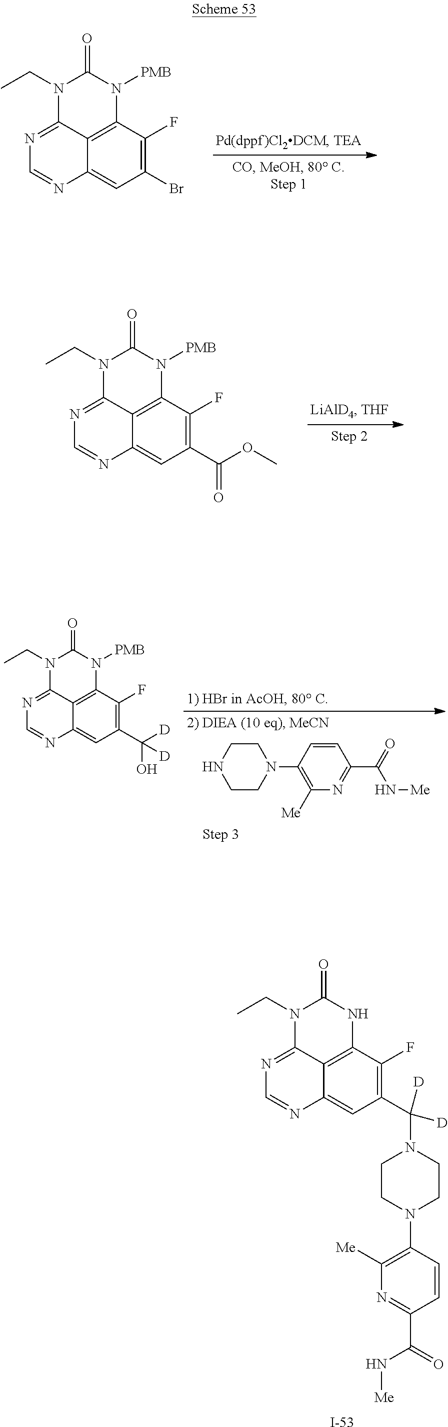

### Step 1: Methyl 3-ethyl-9-fluoro-1-(4-methoxybenzyl)-2-oxo-2,3-dihydro-1H-pyrimido[4,5,6-de]quinazoline-8-carboxylate

The mixture of 8-bromo-3-ethyl-9-fluoro-1-(4-methoxybenzyl)-1H-pyrimido[4,5,6-de]quinazolin-2(3H)-one (Example 35, Step 6:9 g, 20.87 mmol), triethylamine (10.56 g, 104.35 mmol) and bis(diphenylphosphino)ferrocene-palladium(II) dichloride dichloromethane complex (0.17 g, 0.21 mmol) in methanol (250 mL) was stirred at 80° C. for 16 h under carbon monoxide atmosphere (10 atm). Upon cooling to room temperature, the reaction mixture was concentrated under reduced pressure. The residue was purified by silica gel column chromatography, eluted with 10% methanol in dichloromethane to afford the desired product as a brown solid (8 g, 84%). LCMS calculated for C₂₁H₂₀FN₄O₄ (M+H)⁺ m/z=411.1; found 411.1; ¹H NMR (300 MHz, CDCl₃) δ 8.79 (s, 1H), 7.93 (d, J=5.1 Hz, 1H), 7.32-7.29 (m, 2H), 6.89-6.83 (m, 2H), 5.51 (d, J=2.4 Hz, 2H), 4.39 (q, J=6.9 Hz, 2H), 3.99 (s, 3H), 3.79 (s, 3H), 1.39 (t, J=6.9 Hz, 3H).

### Step 2:3-Ethyl-9-fluoro-8-(hydroxymethyl-d2)-1-(4-methoxybenzyl)-1H-pyrimido[4,5,6-de]quinazolin-2(3H)-one

The mixture of methyl 3-ethyl-9-fluoro-1-(4-methoxybenzyl)-2-oxo-2,3-dihydro-1H-pyrimido[4,5,6-de]quinazoline-8-carboxylate (50 mg, 0.12 mmol) in anhydrous tetrahydrofuran (2 mL) was treated with lithium aluminum deuteride (1 M in tetrahydrofuran, 0.073 mL, 0.073 mmol) at 0° C. under nitrogen atmosphere. After stirring for another 30 min at 0° C., the reaction was quenched with a saturated aqueous solution of ammonium chloride, and concentrated under reduced pressure. The residue was purified by silica gel column chromatography, eluted with 5% methanol in dichloromethane to afford the desired product as a white solid (45 mg, 96%). LCMS calculated for C₂₀H₁₈D₂FN₄O₃ (M+H)⁺ m/z=385.2; found 385.1.

### Step 3: 5-(4-((3-Ethyl-9-fluoro-2-oxo-2,3-dihydro-1H-pyrimido[4,5,6-de]quinazolin-8-yl)methyl-d₂)piperazin-1-yl)-N,6-dimethylpicolinamide (I-53)

3-Ethyl-9-fluoro-8-(hydroxymethyl-d₂)-1-(4-methoxybenzyl)-1H-pyrimido[4,5,6-de]quinazolin-2(3H)-one (90 mg, 0.23 mmol) was treated with hydrogen bromide (33 wt. % solution in glacial acid, 3 mL) at room temperature. The mixture was stirred at 80° C. under nitrogen atmosphere for 2 h. Upon cooling to room temperature, the reaction mixture was concentrated under reduced pressure. To the residue was added acetonitrile (3 mL), followed by N,6-dimethyl-5-(piperazin-1-yl)picolinamide (104 mg, 0.44 mmol) and N-ethyl-N-isopropylpropan-2-amine (440 mg, 3.41 mmol). The resulting mixture was stirred at room temperature for 16 h, and concentrated under reduced pressure. The residue was purified by silica gel column chromatography, eluted with 10% methanol in dichloromethane to give the crude product which was further purified by reversed-phase flash chromatography with the following conditions: column, C18 silica gel; mobile phase, acetonitrile in water (0.1% trifluoroacetic acid), 10% to 50% over 10 min; detector, UV 254 nm. The collected fractions were lyophilized to afford the TFA salt of the desired product as a white solid (50 mg). LCMS calculated for C₂₄H₂₆D₂FN₈O₂ (M+H)⁺ m/z=481.2; found 481.2; ¹H NMR (400 MHz, CD₃OD) δ 8.81 (s, 1H), 7.91 (d, J=8.4 Hz, 1H), 7.60-7.56 (m, 2H), 4.32 (q, J=7.2 Hz, 2H), 3.65-3.57 (m, 4H), 3.29-3.25 (m, 4H), 2.94 (s, 3H), 2.60 (s, 3H), 1.34 (t, J=7.2 Hz, 3H).

### Example 54: 5-(4-((9-Ethyl-6-fluoro-3-methyl-8-oxo-8,9-dihydro-7H-pyridazino[3,4,5-de]quinazolin-5-yl)methyl)piperazin-1-yl)-N,6-dimethylpicolinamide (I-54)

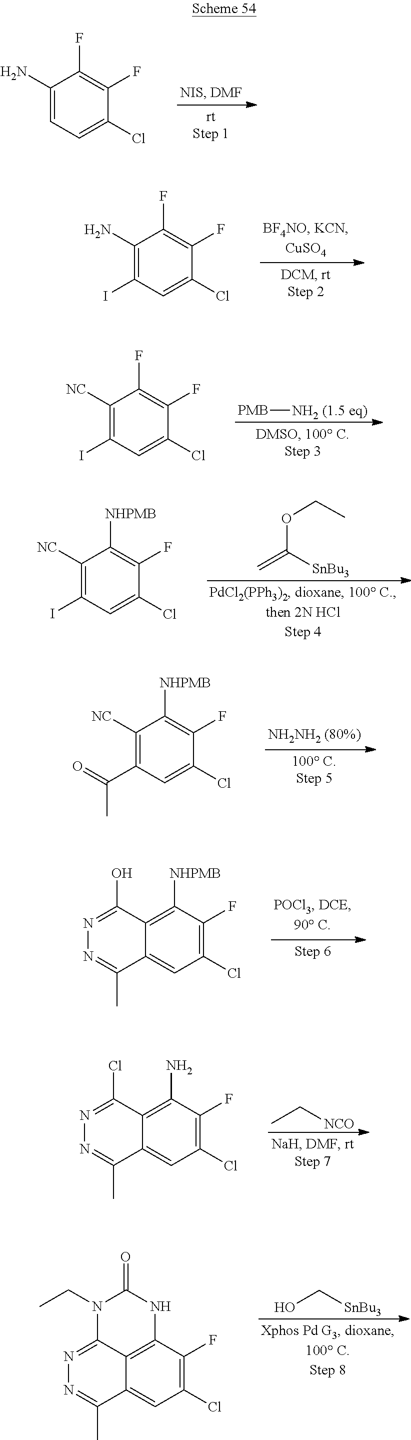

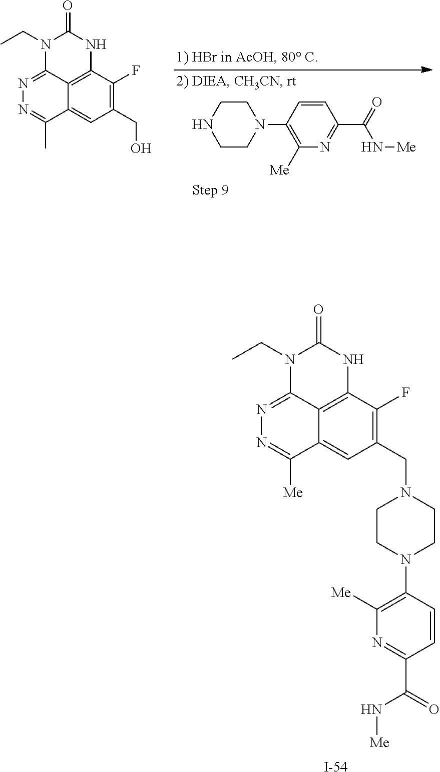

### Step 1: 4-Chloro-2,3-difluoro-6-iodoaniline

To a solution of 4-chloro-2,3-difluoroaniline (5 g, 30.57 mmol) in N,N-dimethylformamide (100 mL) was added 1-iodopyrrolidine-2,5-dione (20.63 g, 91.72 mmol) at room temperature. The mixture was stirred for additional 2 h and then diluted with water (100 mL). The aqueous layer was extracted with ethyl acetate (3×100 mL). The combined organic layers were washed with brine (2×50 mL), dried over anhydrous sodium sulfate. After filtration, the filtrate was concentrated under reduced pressure. The residue was purified by silica gel column chromatography, eluted with 10% ethyl acetate in petroleum ether to give the desired product as a yellow oil (6 g, 68%). ¹H NMR (300 MHz, DMSO-d₆) δ 7.63 (dd, J=7.5, 2.4 Hz, 1H), 5.69 (s, 2H).

### Step 2: 4-Chloro-2,3-difluoro-6-iodobenzonitrile

To a solution of 4-chloro-2,3-difluoro-6-iodoaniline (23 g, 79.46 mmol) in dichloromethane (250 mL) was added nitrosonium tetrafluoroborate (10.21 g, 87.41 mmol, 1.1) at room temperature. After stirring at room temperature for 1 h, the reaction mixture was cooled to 0° C. Potassium cyanide (10.35 g, 158.92 mmol) was added in portions. Then a solution of cupric sulfate (25.36 g, 158.92 mmol) in water (100 mL) was added dropwise over 30 min. After stirring for additional 40 min at 0° C., the mixture was allowed to warm to room temperature and stirred for 1 h. The mixture was diluted with dichloromethane (200 mL) and cooled to 0° C. A saturated aqueous sodium bicarbonate solution (200 mL) was added. The separated aqueous layer was extracted with dichloromethane (3×300 mL). The combined organic layers were washed with brine (2×300 mL), dried over anhydrous sodium sulfate. After filtration, the filtrate was concentrated under reduced pressure. The residue was purified by silica gel column chromatography, eluted with 10% ethyl acetate in petroleum ether to give the desired product as an orange solid (7.5 g, 32%). ¹H NMR (300 MHz, CDCl₃) δ 7.81 (dd, J=6.0, 2.1 Hz, 1H).

### Step 3: 4-Chloro-3-fluoro-6-iodo-2-((4-methoxybenzyl)amino)benzonitrile

To a solution of 4-chloro-2,3-difluoro-6-iodobenzonitrile (2.4 g, 8.02 mmol) in dimethyl sulfoxide (25 mL) was added (4-methoxyphenyl) methanamine (1.65 g, 12.02 mmol) at room temperature. The mixture was stirred at 100° C. for 1 h. After cooling to room temperature, the mixture was diluted with water (300 mL) and extracted with ethyl acetate (3×300 mL). The combined organic layers were washed with brine (3×100 mL), dried over anhydrous sodium sulfate. After filtration, the filtrate was concentrated. The residue was purified by reversed-phase flash chromatography with the following conditions: column, C18 silica gel; mobile phase, acetonitrile in Water (10 mmol/L ammonium bicarbonate), 70% to 90% over 20 min; detector, UV 254 nm. The fractions were collected and concentrated to give the desired product as a yellow solid (2 g, 60%). ¹H NMR (300 MHz, CDCl₃) δ 7.28-7.24 (m, 2H), 7.21 (d, J=6.0 Hz, 1H), 6.91-6.87 (m, 2H), 4.64 (s, 2H), 3.81 (s, 3H).

### Step 4: 6-Acetyl-4-chloro-3-fluoro-2-((4-methoxybenzyl)amino)benzonitrile

To a screw-cap vial equipped with a magnetic stir bar was added 4-chloro-3-fluoro-6-iodo-2-((4-methoxybenzyl)amino)benzonitrile (500 mg, 1.2 mmol), bis(triphenylphosphine)palladium(II) dichloride (126 mg, 0.18 mmol) and tributyl(1-ethoxyethenyl) stannane (650 mg, 1.8 mmol). The vial was sealed with a Teflon-lined septum, evacuated and backfilled with nitrogen (this process was repeated a total of three times). 1,4-Dioxane (10 mL) was added. The reaction was stirred at 100° C. for 2 h. After cooling to room temperature, the reaction mixture was treated with hydrogen chloride (2N in water, 1.20 mL, 2.4 mmol). The mixture was stirred at room temperature for 1 h. A saturated aqueous sodium bicarbonate solution (100 mL) was added. The mixture was extracted with ethyl acetate (3×100 mL). The combined organic layers were washed with brine (300 mL), dried over anhydrous sodium sulfate. After filtration, the filtrate was concentrated. The residue was purified by silica gel column chromatography, eluted with 20% ethyl acetate in petroleum ether to give the desired product as a brown solid (300 mg, 75%). LCMS calculated for C₁₇H₁₃ClFN₂O₂ (M−H)⁻ m/z=331.1; found 331.1; ¹H NMR (400 MHz, CDCl₃) δ 7.26-7.22 (m, 3H), 6.90-6.87 (m, 2H), 5.01 (s, 1H), 4.65 (s, 2H), 3.80 (s, 3H), 2.59 (s, 3H).

### Step 5: 6-Chloro-7-fluoro-8-((4-methoxybenzyl)amino)-4-methylphthalazin-1-ol

The solution of 6-acetyl-4-chloro-3-fluoro-2-((4-methoxybenzyl)amino)benzonitrile (500 mg, 1.5 mmol) in hydrazine hydrate (80% in water, 5 mL) was stirred at 110° C. for 1 h. After cooling to room temperature, the mixture was diluted with water (50 mL) and extracted with dichloromethane (3×50 mL). The combined organic layers were dried over anhydrous sodium sulfate. After filtration, the filtrate was concentrated. The residue was purified by silica gel column chromatography, eluted with 20% ethyl acetate in dichloromethane to give the desired product as an off-white solid (380 mg, 73%). LCMS calculated for C₁₇H₁₆ClFN₃O₂ (M+H)⁺ m/z=348.1; found 348.0; ¹H NMR (400 MHz, DMSO-d₆) δ 12.40 (s, 1H), 9.62-9.58 (m, 1H), 7.27-7.24 (m, 2H), 7.13 (d, J=6.4 Hz, 1H), 6.91-6.89 (m, 2H), 4.62-4.59 (m, 2H), 3.72 (s, 3H), 2.38 (s, 3H).

### Step 6:4,7-Dichloro-6-fluoro-1-methylphthalazin-5-amine

To a solution of 6-chloro-7-fluoro-8-((4-methoxybenzyl)amino)-4-methylphthalazin-1-ol (300 mg, 0.86 mmol) in 1,2-dichloroethane (20 mL) was added phosphoryl trichloride (6.35 g, 41.42 mmol) dropwise at room temperature. After stirring at 90° C. for 1 h, the mixture was cooled to room temperature and concentrated. To the residue was added ethyl acetate (50 mL) followed by saturated aqueous sodium bicarbonate (50 mL). The separated aqueous layer was extracted with ethyl acetate (3×50 mL). The combined organic layers were dried over anhydrous sodium sulfate. After filtration, the filtrate was concentrated. The residue was purified by silica gel column chromatography, eluted with 15% ethyl acetate in dichloromethane to give the desired product as a light-yellow solid (120 mg, 56%). LCMS calculated for C₉H₇C₁₂FN₃ (M+H)⁺ m/z=246.0; found 246.2.

### Step 7: 5-Chloro-9-ethyl-6-fluoro-3-methyl-7H-pyridazino[3,4,5-de]quinazolin-8 (9H)-one

To a solution of 4,7-dichloro-6-fluoro-1-methylphthalazin-5-amine (120 mg, 0.49 mmol) in N,N-dimethylformamide (3 mL) was added sodium hydride (60% dispersion in mineral oil, 39 mg, 0.98 mmol) at 0° C. After stirring for 30 min, the mixture was treated with isocyanatoethane (52 mg, 0.73 mmol). The resulting mixture was stirred at room temperature for 1 h and then quenched with saturated aqueous ammonium chloride (10 mL). The mixture was extracted with ethyl acetate (3×30 mL). The combined organic layers were dried over anhydrous sodium sulfate. After filtration, the filtrate was concentrated. The residue was purified by Prep-TLC (35% ethyl acetate in dichloromethane) to give the desired product as a light brown solid (70 mg, 51%). LCMS calculated for C₁₂H₁₁ClFN₄O (M+H)⁺ m/z=281.1; found 281.1.

### Step 8:9-Ethyl-6-fluoro-5-(hydroxymethyl)-3-methyl-7H-pyridazino[3,4,5-de]quinazolin-8 (9H)-one

To a screw-cap vial equipped with a magnetic stir bar was added 5-chloro-9-ethyl-6-fluoro-3-methyl-7H-pyridazino[3,4,5-de]quinazolin-8 (9H)-one (60 mg, 0.21 mmol), XPhos Pd G3 (36 mg, 0.04 mmol) and (tributylstannyl)methanol (103 mg, 0.32 mmol). The vial was sealed with a Teflon-lined septum, evacuated and backfilled with nitrogen (this process was repeated a total of three times). 1,4-Dioxane (3 mL) was added. The resulting mixture was stirred at 100° C. for 1 h. After cooling to room temperature, the reaction was concentrated. The residue was purified by reversed-phase flash chromatography with the following conditions: column, C18 silica gel; mobile phase, acetonitrile in water (0.1% formic acid), 5% to 50% gradient over 20 min; detector, UV 254 nm. The fractions were collected and concentrated to give the desired product as a white solid (50 mg, 85%). LCMS calculated for C₁₃H₁₄FN402 (M+H)⁺ m/z=277.1; found 277.2.

### Step 9: 5-(4-((9-Ethyl-6-fluoro-3-methyl-8-oxo-8,9-dihydro-7H-pyridazino[3,4,5-de]quinazolin-5-yl)methyl)piperazin-1-yl)-N,6-dimethylpicolinamide (I-54)

To 9-ethyl-6-fluoro-5-(hydroxymethyl)-3-methyl-7H-pyridazino[3,4,5-de]quinazolin-8 (9H)-one (25 mg, 0.09 mmol) was added hydrogen bromide (33 wt. % solution in glacial acid, 1 mL) at room temperature. The reaction mixture was stirred at 80° C. under nitrogen atmosphere for 2 h. Upon cooling to room temperature, the reaction mixture was concentrated under reduced pressure. To the residue was added acetonitrile (2 mL), followed by N,6-dimethyl-5-(piperazin-1-yl)picolinamide (23 mg, 0.1 mmol), and N-ethyl-N-isopropylpropan-2-amine (117 mg, 0.9 mmol). The resulting mixture was stirred at room temperature for 16 h; and then concentrated under vacuum. The residue was purified by reversed-phase flash chromatography (C18 silica gel; mobile phase, acetonitrile in water (0.1% trifluoroacetic acid), 10% to 50% gradient over 20 min; detector, UV 254 nm. The collected fractions were lyophilized to provide the TFA salt of the desired product as a white solid (13.6 mg). LCMS calculated for C₂₅H₃₀FN₈O₂ (M+H)⁺ m/z=493.2; found 493.3; ¹H NMR (400 MHz, CD₃OD) δ 8.15 (d, J=7.2 Hz, 1H), 7.89 (d, J=8.4 Hz, 1H), 7.56 (d, J=8.0 Hz, 1H), 4.67 (s, 2H), 4.29 (q, J=7.2 Hz, 2H), 3.52-3.49 (m, 4H), 3.29-3.26 (m, 4H), 2.96 (s, 3H), 2.94 (s, 3H), 2.59 (s, 3H), 1.36 (t, J=7.2 Hz, 3H).

### Example 55: N-cyclopropyl-5-(4-((3-ethyl-9-fluoro-2-oxo-2,3-dihydro-1H-pyrimido[4,5,6-de]quinazolin-8-yl)methyl)piperazin-1-yl)-6-methylpicolinamide (I-55)

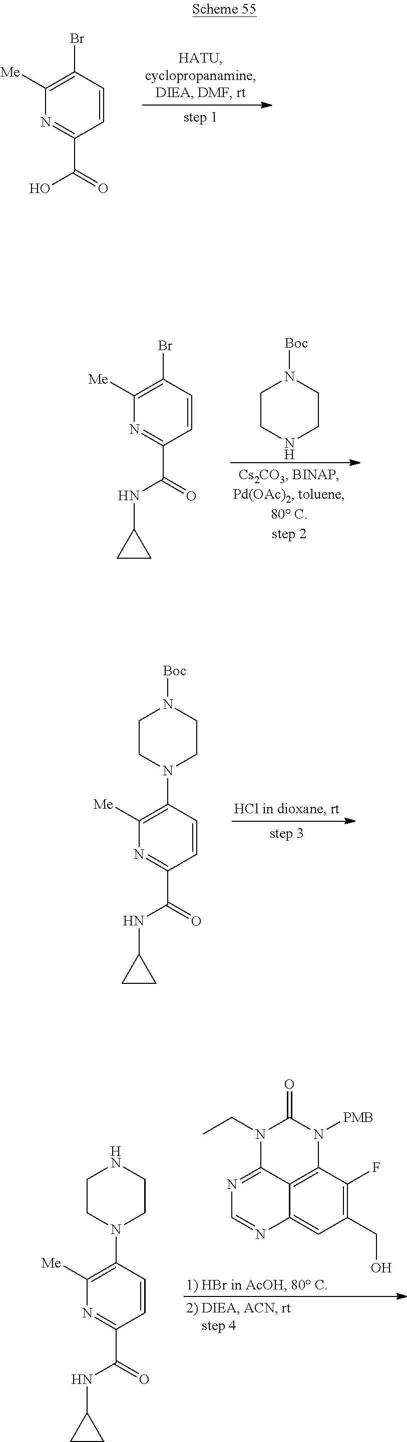

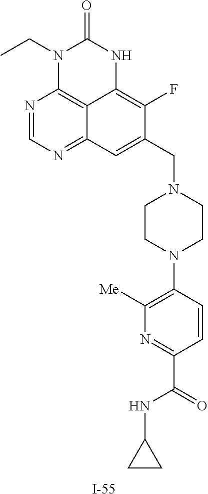

### Step 1: 5-Bromo-N-cyclopropyl-6-methylpicolinamide

To a solution of 5-bromo-6-methylpicolinic acid (2 g, 9.26 mmol) in N,N-dimethylformamide (30 mL) was added 2-(7-azabenzotriazol-1-yl)-N,N,N′,N′-tetramethyluronium hexafluorophosphate (4.22 g, 11.11 mmol). The mixture was stirred at room temperature for 30 min. N-ethyl-N-isopropylpropan-2-amine (5.98 g, 46.29 mmol) was added followed by cyclopropanamine (0.79 g, 13.89 mmol). After stirring at room temperature for 2 h, the reaction was diluted with water (300 mL) and extracted with ethyl acetate (3×150 mL). The combined organic layers were washed with brine (800 mL), dried over anhydrous sodium sulfate. After filtration, the filtrate was concentrated. The residue was purified by silica gel column chromatography, eluted with 30% ethyl acetate in petroleum ether to give the desired product as a yellow oil (1.83 g, 77%). LCMS calculated for C₁₀H₁₂BrN₂O (M+H)⁺ m/z=255.0; found 255.0.

### Step 2: Tert-butyl 4-(6-(cyclopropylcarbamoyl)-2-methylpyridin-3-yl)piperazine-1-carboxylate

To a round bottom flask equipped with a magnetic stir bar was added 5-bromo-N-cyclopropyl-6-methylpicolinamide (1.83 g, 7.17 mmol), cesium carbonate (4.67 g, 14.35 mmol), racemic-2,2′-bis(diphenylphosphino)-1,1′-binaphthyl (0.67 g, 1.07 mmol), palladium acetate (0.16 g, 0.72 mmol) and tert-butyl piperazine-1-carboxylate (2 g, 10.76 mmol). The flask was sealed with a rubber septum, evacuated and backfilled nitrogen (this process was repeated a total of three times). Toluene (20 mL) was added. The reaction was heated at 80° C. for 16 h. After cooling to room temperature, the mixture was concentrated. The residue was purified by silica gel column chromatography, eluted with 30% ethyl acetate in petroleum ether to give the desired product as a brown yellow oil (2.25 g, 87%). LCMS calculated for C₁₉H₂₉N₄O₃ (M+H)⁺ m/z=361.2; found 361.2; ¹H NMR (400 MHz, CDCl₃) δ 7.99 (d, J=8.4 Hz, 1H), 7.32 (d, J=8.4 Hz, 1H), 3.61-3.59 (m, 4H), 2.91-2.89 (m, 5H), 2.52 (s, 3H), 1.49 (s, 9H), 0.89-0.84 (m, 2H), 0.68-0.64 (m, 2H).

### Step 3: N-cyclopropyl-6-methyl-5-(piperazin-1-yl)picolinamide

The mixture of tert-butyl 4-(6-(cyclopropylcarbamoyl)-2-methylpyridin-3-yl)piperazine-1-carboxylate (2.25 g, 6.24 mmol) in hydrogen chloride (4 M in 1,4-dioxane, 30 mL) was stirred at room temperature for 1 h. The resulting mixture was concentrated. The residue was treated with saturated aqueous sodium bicarbonate (100 mL) and extracted with dichloromethane (5×80 mL). The combined organic layers were dried over anhydrous sodium sulfate and filtered. The filtrate was concentrated under reduced pressure. The residue was purified by silica gel column chromatography, eluted with 10% ammonia (7 N in methanol) in dichloromethane to give the desired product as a light-yellow solid (1.58 g, 97%). LCMS calculated for C₁₄H₂₁N₄O (M+H)⁺ m/z=261.2; found 261.2.

### Step 4: N-cyclopropyl-5-(4-((3-ethyl-9-fluoro-2-oxo-2,3-dihydro-1H-pyrimido[4,5,6-de]quinazolin-8-yl)methyl)piperazin-1-yl)-6-methylpicolinamide (1-55)

To 3-ethyl-9-fluoro-8-(hydroxymethyl)-1-(4-methoxybenzyl)-1H-pyrimido[4,5,6-de]quinazolin-2(3H)-one (Example 35, Step 7:150 mg, 0.46 mmol) was added hydrogen bromide (33 wt. % solution in glacial acid, 4 mL) at room temperature. The reaction mixture was stirred at 80° C. under nitrogen atmosphere for 2 h. Upon cooling to room temperature, the reaction mixture was concentrated under reduced pressure. To the residue was added acetonitrile (5 mL), followed by N-cyclopropyl-6-methyl-5-(piperazin-1-yl)picolinamide (144.13 mg, 0.55 mmol) and N-ethyl-N-isopropylpropan-2-amine (596 mg, 4.61 mmol). The resulting mixture was stirred at room temperature for 16 h; and then concentrated under vacuum. The residue was purified by silica gel column chromatography, eluted with 10% methanol in dichloromethane to give the crude product which was further purified by reversed-phase flash chromatography with the following conditions: column, C18 silica gel; mobile phase, acetonitrile in water (0.1% trifluoroacetic acid), 10% to 50% over 10 min; detector, UV 254 nm. The collected fractions were lyophilized to afford the TFA salt of the desired product as a white solid (192.5 mg). LCMS calculated for C₂₆H₃₀FN₈O₂ (M+H)⁺ m/z=505.2; found 505.3; ¹H NMR (400 MHz, CD₃OD) δ 8.77 (s, 1H), 7.91 (d, J=8.4 Hz, 1H), 7.58-7.56 (m, 2H), 4.70 (s, 2H), 4.31 (q, J=7.2 Hz, 2H), 3.63-3.58 (m, 4H), 3.29-3.25 (m, 4H), 2.88-2.82 (m, 1H), 2.58 (s, 3H), 1.33 (t, J=7.2 Hz, 3H), 0.86-0.81 (m, 2H), 0.68-0.64 (m, 2H).

### Example 56: N-cyclopropyl-5-(4-((9-ethyl-6-fluoro-3-methyl-8-oxo-8,9-dihydro-7H-pyridazino[3,4,5-de]quinazolin-5-yl)methyl)piperazin-1-yl)-6-methylpicolinamide (I-56)

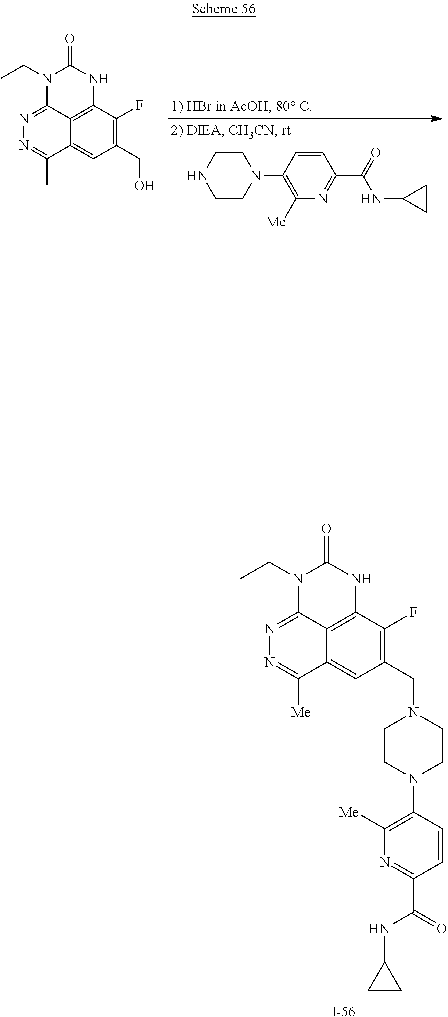

To 9-ethyl-6-fluoro-5-(hydroxymethyl)-3-methyl-7H-pyridazino[3,4,5-de]quinazolin-8 (9H)-one (Example 54, Step 8:25 mg, 0.09 mmol) was added hydrogen bromide (33 wt. % solution in glacial acid, 1 mL) at room temperature. The reaction mixture was heated at 80° C. under nitrogen atmosphere for 2 h. Upon cooling to room temperature, the reaction mixture was concentrated under reduced pressure. To the residue was added acetonitrile (3 mL); followed by N-cyclopropyl-6-methyl-5-(piperazin-1-yl)picolinamide (Example 55, Step 3:26 mg, 0.1 mmol) and N-ethyl-N-isopropylpropan-2-amine (117 mg, 0.9 mmol). The resulting mixture was stirred at room temperature for 16 h, and then concentrated under vacuum. The residue was purified by silica gel column chromatography, eluted with 10% methanol in dichloromethane give the crude product which was further purified by reversed-phase flash chromatography with the following conditions: column, C18 silica gel; mobile phase, acetonitrile in water (0.1% trifluoroacetic acid), 10% to 50% over 20 min; detector, UV 254 nm. The collected fractions were lyophilized to afford the TFA salt of the desired product as a white solid (8.5 mg). LCMS calculated for C₂₇H₃₂FN₈O₂ (M+H)⁺ m/z=519.3; found 519.3; ¹H NMR (400 MHz, CD₃OD) δ 8.15 (d, J=5.6 Hz, 1H), 7.90 (d, J=8.0 Hz, 1H), 7.56 (d, J=8.0 Hz, 1H), 4.65 (s, 2H), 4.29 (q, J=7.2 Hz, 2H), 3.51-3.48 (m, 4H), 3.29-3.26 (m, 4H), 2.96 (s, 3H), 2.88-2.82 (m, 1H), 2.58 (s, 3H), 1.35 (t, J=7.2 Hz, 3H), 0.87-0.81 (m, 2H), 0.67-0.64 (m, 2H).

### Example 57: 5-(4-((3-Ethyl-9-fluoro-2-oxo-2,3-dihydro-1H-pyrimido[4,5,6-de]quinazolin-8-yl)methyl)piperazin-1-yl)-N-methoxy-6-methylpicolinamide (I-57)

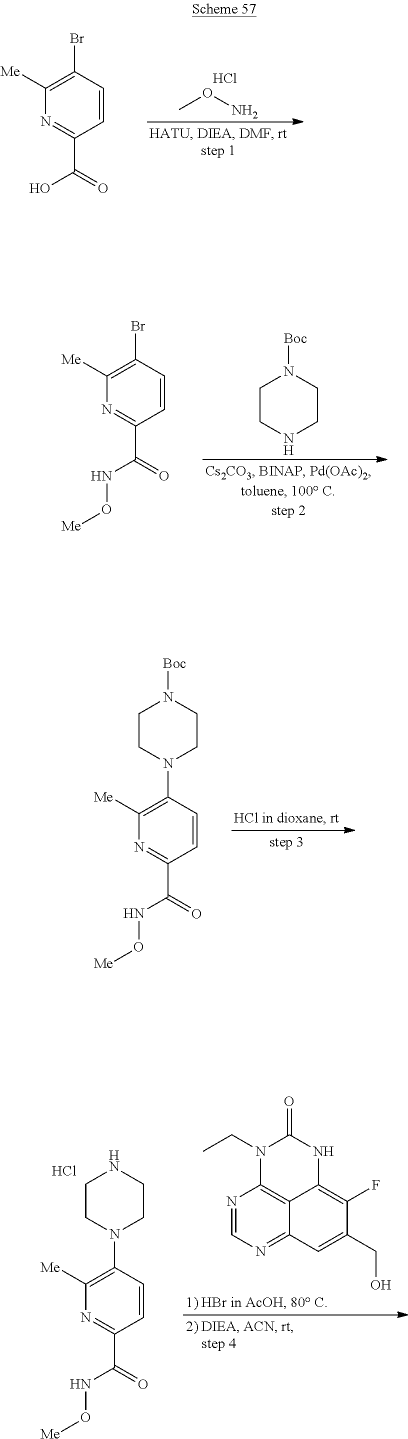

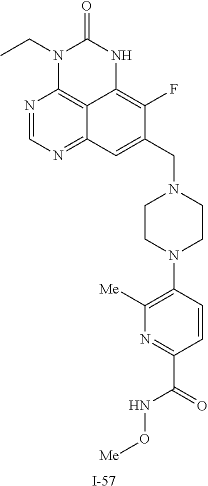

### Step 1: 5-Bromo-N-methoxy-6-methylpicolinamide

To a solution of 5-bromo-6-methylpicolinic acid (1 g, 4.63 mmol) in N,N-dimethylformamide (10 mL) was added 2-(7-azabenzotriazol-1-yl)-N,N,N′,N′-tetramethyluronium hexafluorophosphate (2.11 g, 5.56 mmol). The mixture was stirred at room temperature for 30 min. N-ethyl-N-isopropylpropan-2-amine (2.99 g, 23.15 mmol) was added followed by O-methylhydroxylamine hydrochloride (0.58 g, 6.94 mmol). After stirring at room temperature for 2 h, the reaction was diluted with water (50 mL) and extracted with ethyl acetate (3×30 mL). The combined organic layers were washed with brine (50 mL), dried over anhydrous sodium sulfate. After filtration, the filtrate was concentrated. The residue was purified by silica gel column chromatography, eluted with 30% ethyl acetate in petroleum ether to give the desired product as a yellow solid (0.9 g, 80%). LCMS calculated for C₈H₁₀BrN₂O₂ (M+H)⁺ m/z=245.0; found 245.0.

### Step 2: Tert-butyl 4-(6-(methoxycarbamoyl)-2-methylpyridin-3-yl)piperazine-1-carboxylate

To a round bottom flask equipped with a magnetic stir bar was added 5-bromo-N-methoxy-6-methylpicolinamide (450 mg, 1.84 mmol), cesium carbonate (1197 mg, 3.67 mmol), racemic-2,2′-bis(diphenylphosphino)-1,1′-binaphthyl (172 mg, 0.28 mmol), palladium acetate (41 mg, 0.18 mmol) and tert-butyl piperazine-1-carboxylate (513 mg, 2.75 mmol). The flask was sealed with a rubber septum, evacuated and backfilled nitrogen (this process was repeated a total of three times). Toluene (5 mL) was added. The reaction was stirred at 100° C. for 16 h. After cooling to room temperature, the mixture was concentrated. The residue was purified by silica gel column chromatography, eluted with 30% ethyl acetate in petroleum ether to give the desired product as a yellow solid (123 mg, 19%). LCMS calculated for C₁₇H₂₇N₄O₄ (M+H)⁺ m/z=351.2; found 351.4.

### Step 3: N-methoxy-6-methyl-5-(piperazin-1-yl)picolinamide hydrochloride

The mixture of tert-butyl 4-(6-(methoxycarbamoyl)-2-methylpyridin-3-yl)piperazine-1-carboxylate (123 mg, 0.35 mmol) in hydrogen chloride (4 M in 1,4-dioxane, 2 mL) was stirred at room temperature for 1 h. The resulting mixture was concentrated to give a white solid (100 mg) which was used directly in the next step without further purification. LCMS calculated for C₁₂H₁₉N₄O₂ (M+H)⁺ m/z=251.2; found 251.3.

### Step 4: 5-(4-((3-Ethyl-9-fluoro-2-oxo-2,3-dihydro-1H-pyrimido[4,5,6-de]quinazolin-8-yl)methyl)piperazin-1-yl)-N-methoxy-6-methylpicolinamide (I-57)

To 3-ethyl-9-fluoro-8-(hydroxymethyl)-1-(4-methoxybenzyl)-1H-pyrimido[4,5,6-de]quinazolin-2(3H)-one (Example 35, Step 7:100 mg, 0.38 mmol) was added hydrogen bromide (33 wt. % solution in glacial acid, 3 mL) at room temperature. The reaction mixture was stirred at 80° C. under nitrogen atmosphere for 2 h. Upon cooling to room temperature, the reaction mixture was concentrated under reduced pressure. To the residue was added acetonitrile (5 mL); followed by N-methoxy-6-methyl-5-(piperazin-1-yl)picolinamide hydrochloride (100 mg, 0.35 mmol) and N-ethyl-N-isopropylpropan-2-amine (492 mg, 3.82 mmol). The resulting mixture was stirred at room temperature for 16 h; and then concentrated under vacuum. The residue was purified by silica gel column chromatography, eluted with 10% methanol in dichloromethane give the crude product which was further purified by reversed-phase flash chromatography with the following conditions: column, C₁₈ silica gel; mobile phase, acetonitrile in water (0.1% trifluoroacetic acid), 10% to 50% over 30 min; detector, UV 254 nm. The collected fractions were lyophilized to afford the TFA salt of the desired product as a white solid (111.8 mg). LCMS calculated for C₂₄H₂₈FN₈O₃ (M+H)⁺ m/z=495.2; found 495.3; ¹H NMR (300 MHz, CD₃OD) δ 8.81 (s, 1H), 7.91 (d, J=8.4 Hz, 1H), 7.62-7.58 (m, 2H), 4.74 (s, 2H), 4.34 (q, J=6.9 Hz, 2H), 3.83 (s, 3H), 3.65-3.61 (m, 4H), 3.31-3.24 (m, 4H), 2.61 (s, 3H), 1.36 (t, J=6.9 Hz, 3H).

### Example 58: N-(cyclopropylmethoxy)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide (I-58)

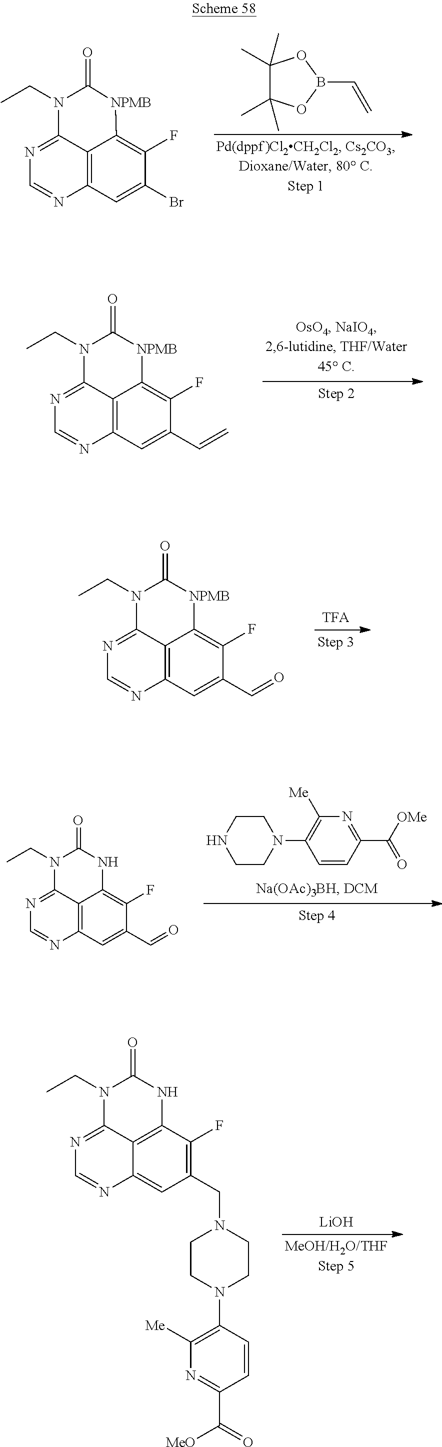

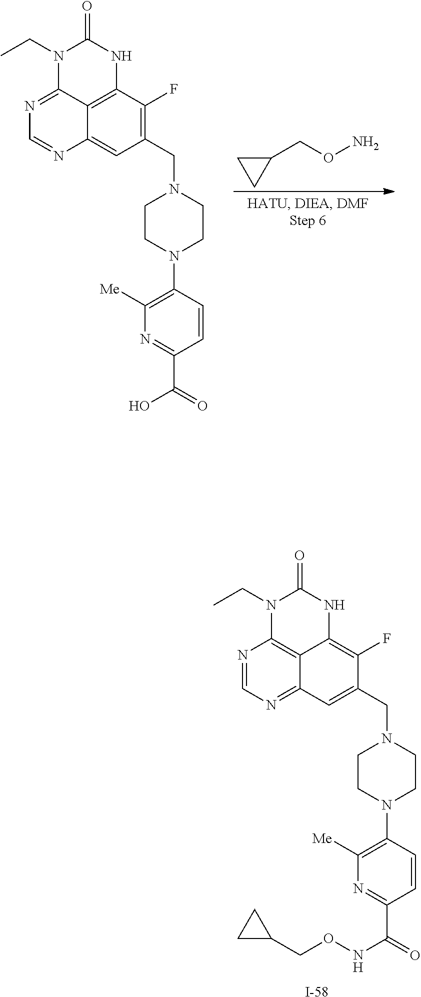

### Step 1:3-Ethyl-9-fluoro-1-(4-methoxybenzyl)-8-vinyl-1H-pyrimido[4,5,6-de]quinazolin-2(3H)-one

To a 500-mL round bottom flask equipped with a magnetic stirring bar was added 8-bromo-3-ethyl-9-fluoro-1-(4-methoxybenzyl)-1H-pyrimido[4,5,6-de]quinazolin-2(3H)-one (Example 35, Step 6:5.85 g, 13.56 mmol), Pd (dppf) C₁₂ CH₂Cl₂ (1.11 g, 1.36 mmol), vinylboronic acid pinacol ester (6.89 g, 27.13 mmol) and cesium carbonate (8.84 g, 27.13 mmol). The flask was sealed with a rubber septum, evacuated and backfilled nitrogen (this process was repeated a total of three times). 1,4-Dioxane (100 mL) was added followed by water (20 mL). The mixture was stirred at 80° C. for 4 h. After cooling to room temperature, the reaction mixture was diluted with water (295 mL). The resulting precipitate was collected by filtration and dried under vacuum. The crude solid was purified by silica gel column chromatography (0-100% EtOAc in hexanes) to provide the desired product as a slight yellow solid (3.00 g, 58%). LCMS calculated for C₂₁H₂₀FN₄O₂ (M+H)⁺ m/z=379.2; found 379.2.

### Step 2:3-Ethyl-9-fluoro-1-(4-methoxybenzyl)-2-oxo-2,3-dihydro-1H-pyrimido[4,5,6-de]quinazoline-8-carbaldehyde

To 3-ethyl-9-fluoro-1-(4-methoxybenzyl)-8-vinyl-1H-pyrimido[4,5,6-de]quinazolin-2(3H)-one (4.70 g, 12.42 mmol) was added THF (90 mL) and water (30 mL) at room temperature. Then sodium periodate (13.28 g, 62.1 mmol) was added followed by osmium tetraoxide (4% in water, 3.25 mL, 0.50 mmol) and 2,6-dimethylpyridine (2.86 mL, 24.84 mmol). The reaction mixture was stirred at 45° C. for 18 hours. After cooling to room temperature, the reaction mixture was diluted with water (200 mL). The resulting precipitate was collected by filtration and washed with water (250 mL). To the precipitate was added water (800 mL). The mixture was stirred at room temperature for 1 hour. The solid was collected by filtration and dry under to provide the desired product as a slight yellow solid (3.92 g, 83%). LCMS calculated for C₂₀H₁₈FN₄O₃ (M+H)⁺ m/z=381.1; found 381.1.

### Step 3:3-Ethyl-9-fluoro-2-oxo-2,3-dihydro-1H-pyrimido[4,5,6-de]quinazoline-8-carbaldehyde

To 3-ethyl-9-fluoro-1-(4-methoxybenzyl)-2-oxo-2,3-dihydro-1H-pyrimido[4,5,6-de]quinazoline-8-carbaldehyde (3.92 g, 10.31 mmol) was added trifluoroacetic acid (100.0 mL) at room temperature. The resulting mixture was stirred at 75° C. for 18 hours before cooling to room temperature. To the reaction mixture was added dichloromethane (50 mL) and water (300 mL). The separated aqueous phase was neutralized with saturated aqueous sodium bicarbonate solution until pH between 7 and 8. The resulting mixture was extracted with 10% methanol in dichloromethane (8× 200 mL). The combined organic layers were concentrated to provide the desired product as a slight yellow solid (1.98 g, 72%). LCMS calculated for C₁₂H₁₀FN₄O₂ (M+H)⁺ m/z=261.1; found 261.1.

### Step 4: Methyl 5-(4-((3-ethyl-9-fluoro-2-oxo-2,3-dihydro-1H-pyrimido[4,5,6-de]quinazolin-8-yl)methyl)piperazin-1-yl)-6-methylpicolinate

To a stirred mixture of 3-ethyl-9-fluoro-2-oxo-2,3-dihydro-1H-pyrimido[4,5,6-de]quinazoline-8-carbaldehyde (817 mg, 3.14 mmol) and methyl 6-methyl-5-(piperazin-1-yl) picolinate (Intermediate 7:923 mg, 3.92 mmol) in dichloromethane (30 mL) was added methanol (5 mL) followed by acetic acid (19 mg, 0.31 mmol). The mixture was stirred for 1 h at room temperature. Then sodium triacetoxyborohydride (2.66 g, 12.56 mmol) was added. The resulting mixture was stirred for additional 16 h, and then was concentrated. The residue was purified by silica gel column chromatography, eluted with 10% MeOH in CH₂Cl₂ to give the desired product as a white solid (950 mg, 63%). LCMS calculated for C24H27FN703 (M+H)⁺ m/z=480.2; found 480.1.

### Step 5: 5-(4-((3-Ethyl-9-fluoro-2-oxo-2,3-dihydro-1H-pyrimido[4,5,6-de]quinazolin-8-yl)methyl)piperazin-1-yl)-6-methylpicolinic acid

To a mixture of methyl 5-(4-((3-ethyl-9-fluoro-2-oxo-2,3-dihydro-1H-pyrimido[4,5,6-de]quinazolin-8-yl)methyl)piperazin-1-yl)-6-methylpicolinate (850 mg, 1.77 mmol) in methanol (5 mL) was added tetrahydrofuran (2 mL) and water (3 mL). Then lithium hydroxide monohydrate (149 mg, 3.55 mmol) was added. The reaction was stirred at 70° C. for 2 h. After cooling to room temperature, the mixture was concentrated. The residue was neutralized with hydrogen chloride (1 M, 3.6 mL) at 0° C. The resulting mixture was concentrated to give the crude desired product as a yellow solid (0.95 g) which was used directly without further purification. LCMS calculated for C₂₃H₂₅FN₇O₃ (M+H)⁺ m/z=466.2; found 466.2.

### Step 6: N-(cyclopropylmethoxy)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide (I-58)

To a vial was added 5-(4-((3-ethyl-9-fluoro-2-oxo-2,3-dihydro-1H-pyrimido[4,5,6-de]quinazolin-8-yl)methyl)piperazin-1-yl)-6-methylpicolinic acid (12.00 mg, 0.026 mmol), O-(cyclopropylmethyl) hydroxylamine hydrochloride (4.78 mg, 0.039 mmol), and [dimethylamino (triazolo[4,5-b]pyridin-3-yloxy)methylene]-dimethyl-ammonium hexafluorophosphate (14.70 mg, 0.039 mmol). DMF (0.5 mL) was added, followed by N,N-diisopropylethylamine (17.0 mg, 0.13 mmol). After stirring at room temperature for 15 min, the reaction mixture was purified by prep-HPLC (Column: Sunfire prep C18 column; mobile phase A: water (0.05% trifluoroacetic acid), mobile Phase B: acetonitrile; flow rate: 60 mL/min; gradient: 5% B to 30% B over 7 min). Eluted fractions were collected and lyophilized to afford the TFA salt of the desired product as a white solid. LCMS calculated for C₂₇H₃₂FN₈O₃ (M+H)⁺ m/z=535.3; found 535.3.

### Examples 59-78

Examples 59-78 in Table 3 of were prepared according to the procedure described in Example 58, using the corresponding amines instead of O-(cyclopropylmethyl) hydroxylamine hydrochloride.

TABLE 3

| | | LCMS |
|---|---|---|
| | | (M + H)⁺ |
| Example | Chemical Structure & Name | m/z |
| 59 | 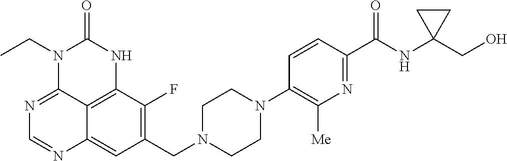 [SMILES: CCN1C(=O)Nc2c(F)c(CN3CCN(c4ccc(C(=O)NC5(CO)CC5)nc4C)CC3)cc3ncnc1c23] 5-(4-((3-ethyl-9-fluoro-2-oxo-2,3-dihydro-1H-pyrimido[4,5,6- de]quinazolin-8-yl)methyl)piperazin-1-yl)-N-(1- (hydroxymethyl)cyclopropyl)-6-methylpicolinamide | 535.3 |
| 60 | 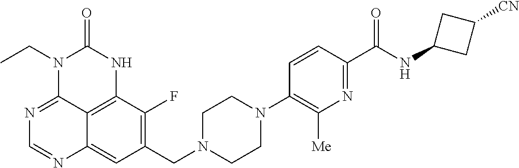 [SMILES: CCN1C(=O)Nc2c(F)c(CN3CCN(c4ccc(C(=O)N[C@H]5C[C@H](C#N)C5)nc4C)CC3)cc3ncnc1c23] N-((1r,3r)-3-cyanocyclobutyl)-5-(4-((3-ethyl-9-fluoro-2-oxo-2,3- dihydro-1H-pyrimido[4,5,6-de]quinazolin-8-yl)methyl)piperazin-1- yl)-6-methylpicolinamide | 544.3 |
| 61 | 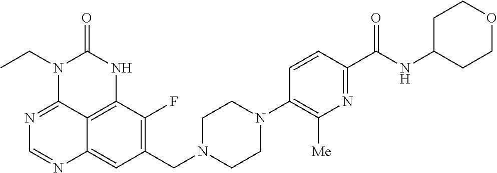 [SMILES: CCN1C(=O)Nc2c(F)c(CN3CCN(c4ccc(C(=O)NC5CCOCC5)nc4C)CC3)cc3ncnc1c23] 5-(4-((3-ethyl-9-fluoro-2-oxo-2,3-dihydro-1H-pyrimido[4,5,6- de]quinazolin-8-yl)methyl)piperazin-1-yl)-6-methyl-N-(tetrahydro- 2H-pyran-4-yl)picolinamide | 549.3 |
| 62 | 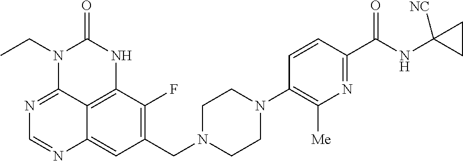 [SMILES: CCN1C(=O)Nc2c(F)c(CN3CCN(c4ccc(C(=O)NC5(C#N)CC5)nc4C)CC3)cc3ncnc1c23] N-(1-cyanocyclopropyl)-5-(4-((3-ethyl-9-fluoro-2-oxo-2,3-dihydro- 1H-pyrimido[4,5,6-de]quinazolin-8-yl)methyl)piperazin-1-yl)-6- methylpicolinamide | 530.2 |
| 63 | 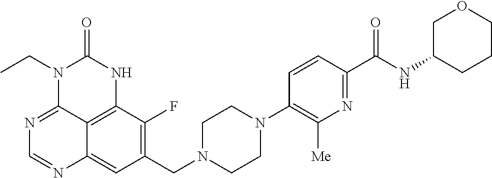 [SMILES: CCN1C(=O)Nc2c(F)c(CN3CCN(c4ccc(C(=O)N[C@H]5CCCOC5)nc4C)CC3)cc3ncnc1c23] (S)-5-(4-((3-ethyl-9-fluoro-2-oxo-2,3-dihydro-1H-pyrimido[4,5,6- de]quinazolin-8-yl)methyl)piperazin-1-yl)-6-methyl-N-(tetrahydro- 2H-pyran-3-yl)picolinamide | 549.3 |
| 64 | 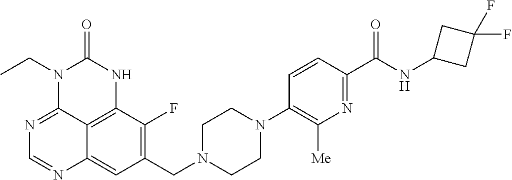 [SMILES: CCN1C(=O)Nc2c(F)c(CN3CCN(c4ccc(C(=O)NC5CC(F)(F)C5)nc4C)CC3)cc3ncnc1c23] N-(3,3-difluorocyclobutyl)-5-(4-((3-ethyl-9-fluoro-2-oxo-2,3- dihydro-1H-pyrimido[4,5,6-de]quinazolin-8-yl)methyl)piperazin-1- yl)-6-methylpicolinamide | 555.2 |
| 65 | 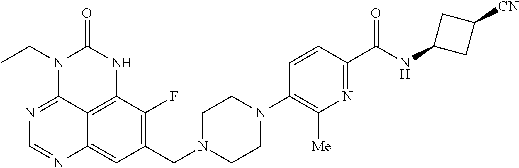 [SMILES: CCN1C(=O)Nc2c(F)c(CN3CCN(c4ccc(C(=O)N[C@H]5C[C@@H](C#N)C5)nc4C)CC3)cc3ncnc1c23] N-((1s,3s)-3-cyanocyclobutyl)-5-(4-((3-ethyl-9-fluoro-2-oxo-2,3- dihydro-1H-pyrimido[4,5,6-de]quinazolin-8-yl)methyl)piperazin-1- yl)-6-methylpicolinamide | 544.3 |
| 66 | 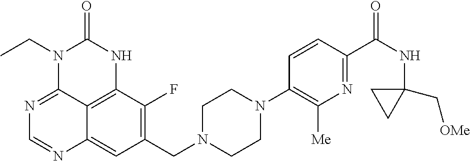 [SMILES: CCN1C(=O)Nc2c(F)c(CN3CCN(c4ccc(C(=O)NC5(COC)CC5)nc4C)CC3)cc3ncnc1c23] 5-(4-((3-ethyl-9-fluoro-2-oxo-2,3-dihydro-1H-pyrimido[4,5,6- de]quinazolin-8-yl)methyl)piperazin-1-yl)-N-(1- (methoxymethyl)cyclopropyl)-6-methylpicolinamide | 549.3 |
| 67 | 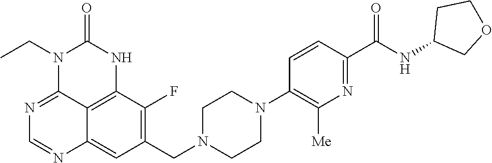 [SMILES: CCN1C(=O)Nc2c(F)c(CN3CCN(c4ccc(C(=O)N[C@@H]5CCOC5)nc4C)CC3)cc3ncnc1c23] (R)-5-(4-((3-ethyl-9-fluoro-2-oxo-2,3-dihydro-1H-pyrimido[4,5,6- de]quinazolin-8-yl)methyl)piperazin-1-yl)-6-methyl-N- (tetrahydrofuran-3-yl)picolinamide | 535.3 |
| 68 | 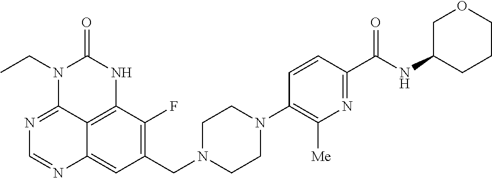 [SMILES: CCN1C(=O)Nc2c(F)c(CN3CCN(c4ccc(C(=O)N[C@@H]5CCCOC5)nc4C)CC3)cc3ncnc1c23] (R)-5-(4-((3-ethyl-9-fluoro-2-oxo-2,3-dihydro-1H-pyrimido[4,5,6- de]quinazolin-8-yl)methyl)piperazin-1-yl)-6-methyl-N-(tetrahydro- 2H-pyran-3-yl)picolinamide | 549.3 |
| 69 | 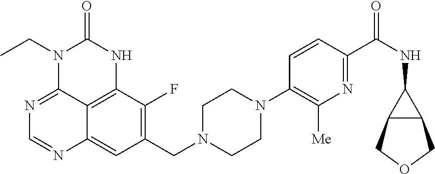 [SMILES: CCN1C(=O)Nc2c(F)c(CN3CCN(c4ccc(C(=O)N[C@@H]5[C@@H]6COC[C@@H]65)nc4C)CC3)cc3ncnc1c23] N-((1R,5S,6s)-3-oxabicyclo[3.1.0]hexan-6-yl)-5-(4-((3-ethyl-9- fluoro-2-oxo-2,3-dihydro-1H-pyrimido[4,5,6-de]quinazolin-8- yl)methyl)piperazin-1-yl)-6-methylpicolinamide | 547.3 |
| 70 | 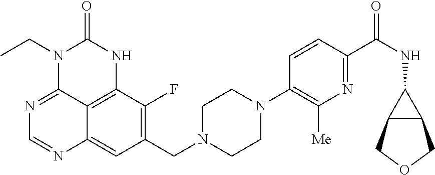 [SMILES: CCN1C(=O)Nc2c(F)c(CN3CCN(c4ccc(C(=O)N[C@H]5[C@@H]6COC[C@@H]65)nc4C)CC3)cc3ncnc1c23] N-((1R,5S,6r)-3-oxabicyclo[3.1.0]hexan-6-yl)-5-(4-((3-ethyl-9- fluoro-2-oxo-2,3-dihydro-1H-pyrimido[4,5,6-de]quinazolin-8- yl)methyl)piperazin-1-yl)-6-methylpicolinamide | 547.3 |
| 71 | 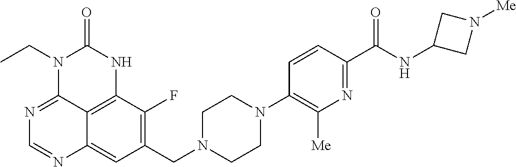 [SMILES: CCN1C(=O)Nc2c(F)c(CN3CCN(c4ccc(C(=O)NC5CN(C)C5)nc4C)CC3)cc3ncnc1c23] 5-(4-((3-ethyl-9-fluoro-2-oxo-2,3-dihydro-1H-pyrimido[4,5,6- de]quinazolin-8-yl)methyl)piperazin-1-yl)-6-methyl-N-(1- methylazetidin-3-yl)picolinamide | 534.3 |
| 72 | 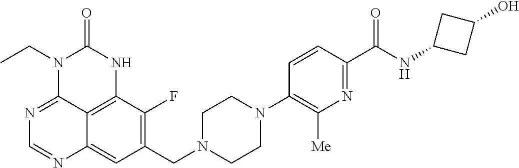 [SMILES: CCN1C(=O)Nc2c(F)c(CN3CCN(c4ccc(C(=O)N[C@H]5C[C@@H](O)C5)nc4C)CC3)cc3ncnc1c23] 5-(4-((3-ethyl-9-fluoro-2-oxo-2,3-dihydro-1H-pyrimido[4,5,6- de]quinazolin-8-yl)methyl)piperazin-1-yl)-N-((1s,3s)-3- hydroxycyclobutyl)-6-methylpicolinamide | 535.3 |
| 73 | 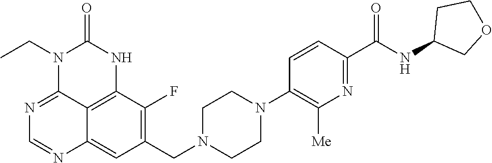 [SMILES: CCN1C(=O)Nc2c(F)c(CN3CCN(c4ccc(C(=O)N[C@H]5CCOC5)nc4C)CC3)cc3ncnc1c23] (S)-5-(4-((3-ethyl-9-fluoro-2-oxo-2,3-dihydro-1H-pyrimido[4,5,6- de]quinazolin-8-yl)methyl)piperazin-1-yl)-6-methyl-N- (tetrahydrofuran-3-yl)picolinamide | 535.3 |
| 74 | 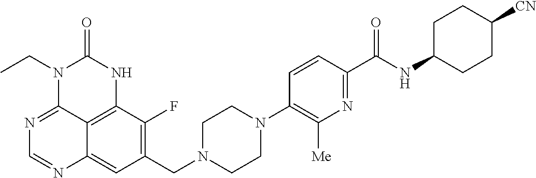 [SMILES: CCN1C(=O)Nc2c(F)c(CN3CCN(c4ccc(C(=O)N[C@H]5CC[C@@H](C#N)CC5)nc4C)CC3)cc3ncnc1c23] N-((1s,4s)-4-cyanocyclohexyl)-5-(4-((3-ethyl-9-fluoro-2-oxo-2,3- dihydro-1H-pyrimido[4,5,6-de]quinazolin-8-yl)methyl)piperazin-1- yl)-6-methylpicolinamide | 572.3 |
| 75 | 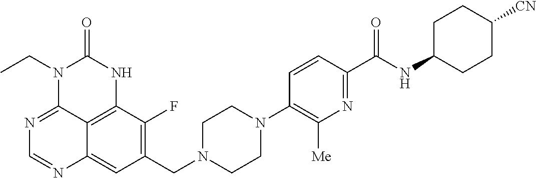 [SMILES: CCN1C(=O)Nc2c(F)c(CN3CCN(c4ccc(C(=O)N[C@H]5CC[C@H](C#N)CC5)nc4C)CC3)cc3ncnc1c23] N-((1r,4r)-4-cyanocyclohexyl)-5-(4-((3-ethyl-9-fluoro-2-oxo-2,3- dihydro-1H-pyrimido[4,5,6-de]quinazolin-8-yl)methyl)piperazin-1- yl)-6-methylpicolinamide | 572.3 |
| 76 | 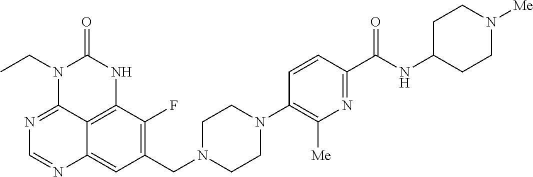 [SMILES: CCN1C(=O)Nc2c(F)c(CN3CCN(c4ccc(C(=O)NC5CCN(C)CC5)nc4C)CC3)cc3ncnc1c23] 5-(4-((3-ethyl-9-fluoro-2-oxo-2,3-dihydro-1H-pyrimido[4,5,6- de]quinazolin-8-yl)methyl)piperazin-1-yl)-6-methyl-N-(1- methylpiperidin-4-yl)picolinamide | 562.3 |
| 77 | 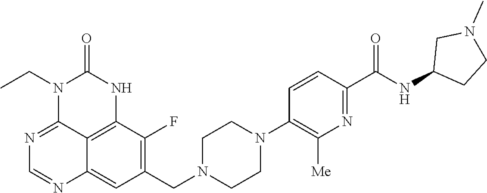 [SMILES: CCN1C(=O)Nc2c(F)c(CN3CCN(c4ccc(C(=O)N[C@@H]5CCN(C)C5)nc4C)CC3)cc3ncnc1c23] (R)-5-(4-((3-ethyl-9-fluoro-2-oxo-2,3-dihydro-1H-pyrimido[4,5,6- de]quinazolin-8-yl)methyl)piperazin-1-yl)-6-methyl-N-(1- methylpyrrolidin-3-yl)picolinamide | 548.3 |
| 78 | 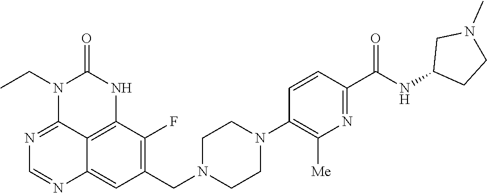 [SMILES: CCN1C(=O)Nc2c(F)c(CN3CCN(c4ccc(C(=O)N[C@H]5CCN(C)C5)nc4C)CC3)cc3ncnc1c23] (S)-5-(4-((3-ethyl-9-fluoro-2-oxo-2,3-dihydro-1H-pyrimido[4,5,6- de]quinazolin-8-yl)methyl)piperazin-1-yl)-6-methyl-N-(1- methylpyrrolidin-3-yl)picolinamide | 548.3 |

### Example 79: N-(1-(cyanomethyl)cyclopropyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide (I-79)

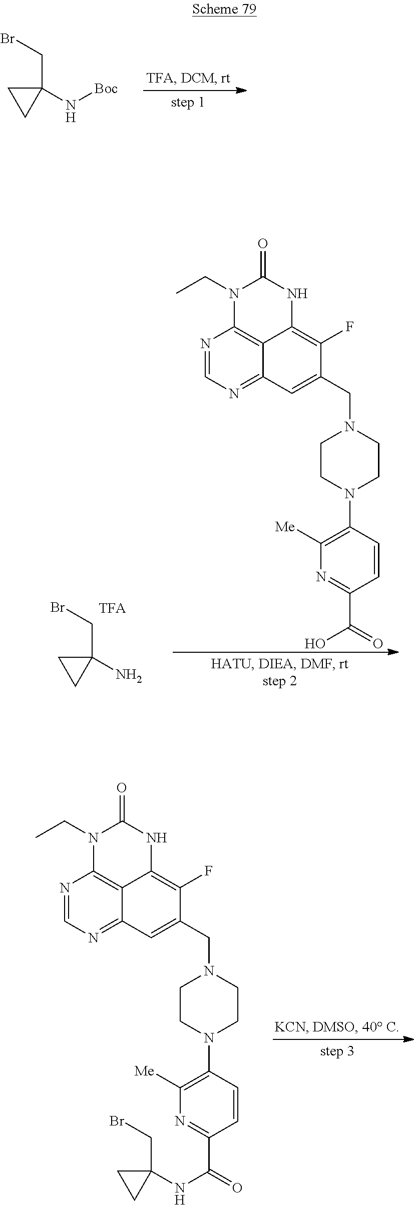 [SMILES: CC(C)(C)OC(=O)NC1(CBr)CC1 ; NC1(CBr)CC1 ; CCN1C(=O)Nc2c(F)c(CN3CCN(c4ccc(C(=O)O)nc4C)CC3)cc3ncnc1c23 ; CCN1C(=O)Nc2c(F)c(CN3CCN(c4ccc(C(=O)NC5(CBr)CC5)nc4C)CC3)cc3ncnc1c23]

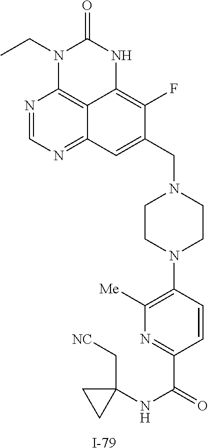

### Step 1: 1-(Bromomethyl)cyclopropan-1-amine

To a mixture of tert-butyl(1-(bromomethyl)cyclopropyl) carbamate (300 mg, 1.2 mmol) in dichloromethane (3 mL) was added 2,2,2-trifluoroacetic acid (1 mL). The mixture was stirred at room temperature for 16 h. The resulting mixture was concentrated to give the TFA salt of the desired product as a light-yellow oil (300 mg).

### Step 2: N-(1-(bromomethyl)cyclopropyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide

To a mixture of 5-(4-((3-ethyl-9-fluoro-2-oxo-2,3-dihydro-1H-pyrimido[4,5,6-de]quinazolin-8-yl)methyl)piperazin-1-yl)-6-methylpicolinic acid (Example 58, Step 5:250 mg, 0.54 mmol) in N,N-dimethylformamide (5 mL) was added 2-(7-azabenzotriazol-1-yl)-N,N,N′,N′-tetramethyluronium hexafluorophosphate (245 mg, 0.64 mmol). The mixture was stirred at room temperature for 15 min. The TFA salt of 1-(bromomethyl)cyclopropan-1-amine (170 mg, 0.64 mmol) was added followed by N-ethyl-N-isopropylpropan-2-amine (347 mg, 2.68 mmol). The resulting mixture was stirred at room temperature for additional 2 h, and then was diluted with water (50 mL). The aqueous layer was extracted with ethyl acetate (3×50 mL). The combined organic layers were washed with brine (80 mL), dried over anhydrous sodium sulfate. After filtration, the filtrate was concentrated. The residue was purified by silica gel column chromatography, eluted with 10% methanol in dichloromethane to give the desired product as a yellow solid (180 mg, 56%). LCMS calculated for C₂₇H₃₁BrFN₈O₂ (M+H)⁺ m/z=597.2; found 596.9.

### Step 3: N-(1-(cyanomethyl)cyclopropyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide (1-79)

To a mixture of N-(1-(bromomethyl)cyclopropyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide (180 mg, 0.30 mmol) in dimethyl sulfoxide (5 mL) was added potassium cyanide (59 mg, 0.9 mmol). The mixture was stirred at 40° C. for 3 h. After cooling to room temperature, the mixture was diluted with water (15 mL) and extracted with ethyl acetate (3×50 mL). The combined organic layers were washed with brine (80 mL), dried over anhydrous sodium sulfate. After filtration, the filtrate was concentrated. The residue was purified by silica gel column chromatography, eluted with 10% methanol in dichloromethane to give the crude product which was further purified by reversed-phase flash chromatography with the following conditions: column, C18 silica gel; mobile phase, acetonitrile in water (0.1% trifluoroacetic acid), 10% to 50% over 30 min; detector, UV 254 nm. The collected fractions were lyophilized to afford the TFA salt of the desired product as a white solid. LCMS calculated for C₂₈H₃₁FN₉O₂ (M+H)⁺ m/z=544.3; found 544.3. ¹H NMR (400 MHz, CD₃OD) δ 8.78 (s, 1H), 7.92 (d, J=8.4 Hz, 1H), 7.58-7.56 (m, 2H), 4.71 (s, 2H), 4.31 (q, J=7.2 Hz, 2H), 3.63-3.59 (m, 4H), 3.29-3.24 (m, 4H), 2.95 (s, 2H), 2.60 (s, 3H), 1.33 (t, J=7.2 Hz, 3H), 1.02-1.00 (m, 4H).

### Example 80: N-cyclopropyl-5-(4-((9-ethyl-6-fluoro-2-methyl-3,8-dioxo-2,7,8,9-tetrahydro-3H-pyridazino[3,4,5-de]quinazolin-5-yl)methyl)piperazin-1-yl)-6-methylpicolinamide (I-80)

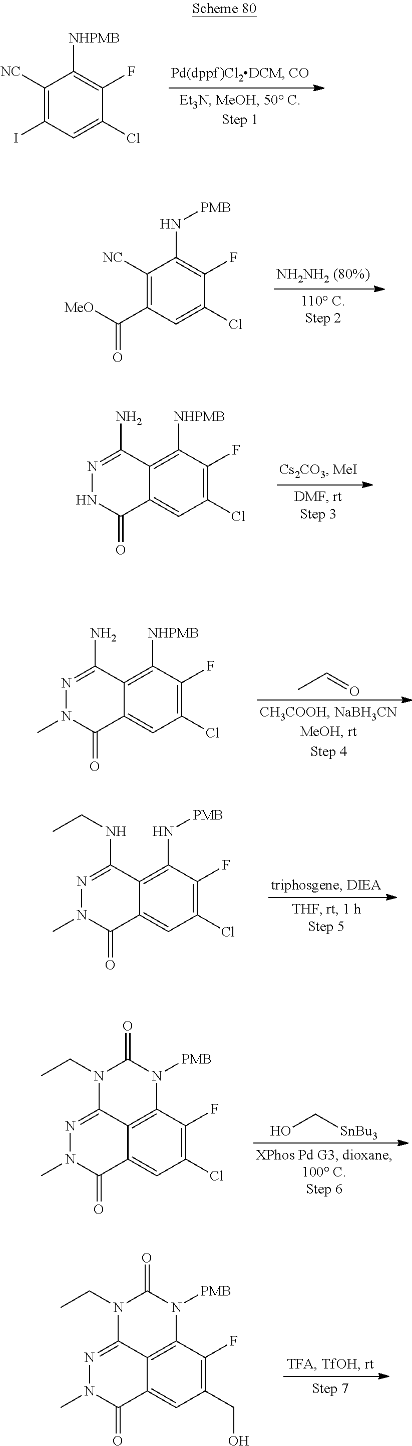

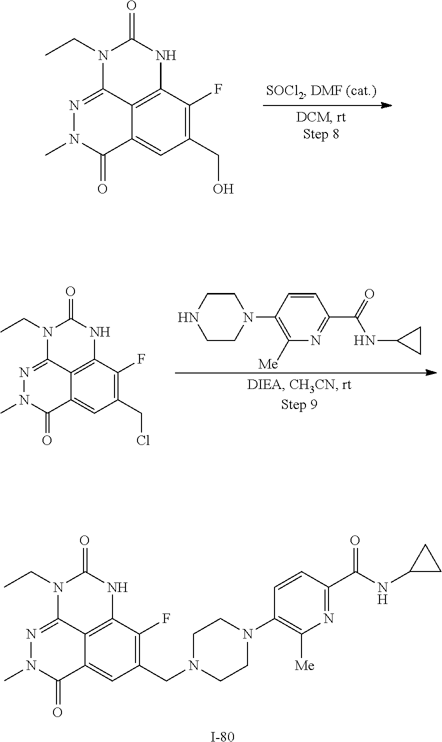

### Step 1: Methyl 5-chloro-2-cyano-4-fluoro-3-((4-methoxybenzyl)amino)benzoate

To a pressure vessel were added 4-chloro-3-fluoro-6-iodo-2-((4-methoxybenzyl)amino)benzonitrile (Example 54, Step 3:1 g, 2.4 mmol), triethylamine (1.21 g, 12 mmol), 1,1′-bis(diphenylphosphino)ferrocene-palladium(II) dichloride dichloromethane complex (196 mg, 0.24 mmol) and methanol (30 mL). The reaction was stirred at 50° C. for 16 h under carbon monoxide atmosphere (10 atm). After cooling to room temperature, the mixture was concentrated under reduced pressure. The residue was purified by silica gel column chromatography, eluted with 20% ethyl acetate/dichloromethane (3/1) in petroleum ether to give the desired product as an orange solid (0.83 g, 99%). ¹H NMR (300 MHz, CDCl₃) δ 7.45 (d, J=6.3 Hz, 1H), 7.27-7.24 (m, 2H), 6.90-6.87 (m, 2H), 4.93 (s, 1H), 4.64 (d, J=2.7 Hz, 2H), 3.95 (s, 3H), 3.80 (s, 3H).

### Step 2: 4-Amino-7-chloro-6-fluoro-5-((4-methoxybenzyl)amino) phthalazin-1 (2H)-one

The mixture of methyl 5-chloro-2-cyano-4-fluoro-3-((4-methoxybenzyl)amino)benzoate (400 mg, 1.15 mmol) in hydrazine hydrate (80% in water, 4 mL) was stirred at 110° C. for 2 h. After cooling to room temperature, the mixture was concentrated under reduced pressure. The residue was purified by silica gel column chromatography, eluted with 20% ethyl acetate in petroleum ether to give the desired product as a brown solid (300 mg, 75%). LCMS calculated for C₁₆H₁₃ClFN₄O₂ (M−H)⁻ m/z=347.1; found 347.0.

### Step 3: 4-Amino-7-chloro-6-fluoro-5-((4-methoxybenzyl)amino)-2-methylphthalazin-1 (2H)-one

To a solution of 4-amino-7-chloro-6-fluoro-5-((4-methoxybenzyl)amino) phthalazin-1 (2H)-one (900 mg, 2.58 mmol) and cesium carbonate (1.01 g, 3.1 mmol) in N,N-dimethylformamide (10 mL) was added methyl iodide (366 mg, 2.58 mmol) dropwise at 0° C. The resulting mixture was stirred at room temperature for 2 h, and then was diluted with water (100 mL). The resulting mixture was extracted with ethyl acetate (3×100 mL). The combined organic layers were washed with brine (3×100 mL), dried over anhydrous sodium sulfate. After filtration, the filtrate was concentrated under reduced pressure. The residue was purified by silica gel column chromatography, eluted with 10% ethyl acetate in dichloromethane to give the desired product as a yellow solid (400 mg, 43%). LCMS calculated for C₁₇H₁₇ClFN₄O₂ (M+H)⁺ m/z=363.1; found 363.2.

### Step 4: 7-Chloro-4-(ethylamino)-6-fluoro-5-((4-methoxybenzyl)amino)-2-methylphthalazin-1 (2H)-one

To a mixture of 4-amino-7-chloro-6-fluoro-5-((4-methoxybenzyl)amino)-2-methylphthalazin-1 (2H)-one (300 mg, 0.83 mmol) in methanol (5 mL) was added acetaldehyde (55 mg, 1.24 mmol). The mixture was stirred at room temperature for 30 min, followed by the addition of acetic acid (99 mg, 1.65 mmol). The resulting mixture was stirred at room temperature for additional 1 h. To the above mixture was added sodium cyanoborohydride (104 mg, 1.65 mmol) at 0° C. The resulting mixture was stirred at room temperature for 16 h, and concentrated under reduced pressure. The residue was purified by silica gel column chromatography, eluted with 20% ethyl acetate in petroleum ether to give the desired product as a yellow solid (100 mg, 31%). LCMS calculated for C₁₉H₂₁ClFN₄O₂ (M+H)⁺ m/z=391.1; found 391.1.

### Step 5: 5-Chloro-9-ethyl-6-fluoro-7-(4-methoxybenzyl)-2-methyl-2,9-dihydro-3H-pyridazino[3,4,5-de]quinazoline-3,8 (7H)-dione

To a mixture of 7-chloro-4-(ethylamino)-6-fluoro-5-((4-methoxybenzyl)amino)-2-methylphthalazin-1 (2H)-one (100 mg, 0.26 mmol) and N-ethyl-N-isopropylpropan-2-amine (66 mg, 0.51 mmol) in anhydrous tetrahydrofuran (0.5 mL) was added triphosgene (76 mg, 0.26 mmol) in portions at 0° C. The resulting mixture was stirred at room temperature for 1 h, and then was quenched with saturated aqueous ammonium chloride. The resulting mixture was extracted with ethyl acetate (4×10 mL). The combined organic layers were dried over anhydrous sodium sulfate. After filtration, the filtrate was concentrated under reduced pressure. The residue was purified by Prep-TLC (20% ethyl acetate in petroleum ether) to give the desired product as a yellow solid (50 mg, 47%). LCMS calculated for C₂₀H₁₉ClFN₄O₃ (M+H)⁺ m/z=417.1; found 417.1.

### Step 6:9-Ethyl-6-fluoro-5-(hydroxymethyl)-7-(4-methoxybenzyl)-2-methyl-2,9-dihydro-3H-pyridazino[3,4,5-de]quinazoline-3,8 (7H)-dione

To a screw-cap vial equipped with a magnetic stir bar was added 5-chloro-9-ethyl-6-fluoro-7-(4-methoxybenzyl)-2-methyl-2,9-dihydro-3H-pyridazino[3,4,5-de]quinazoline-3,8 (7H)-dione (50 mg, 0.12 mmol), XPhos Pd G3 (20 mg, 0.02 mmol) and (tributylstannyl)methanol (77 mg, 0.24 mmol). The vial was sealed with a Teflon-lined septum, evacuated and backfilled with nitrogen (this process was repeated a total of three times). 1,4-Dioxane (2 mL) was added. The resulting mixture was stirred at 100° C. for 1.5 h. After cooling to room temperature, the mixture was concentrated. The residue was purified by silica gel column chromatography, eluted with 30% ethyl acetate in petroleum ether to give the desired product as a yellow oil. LCMS calculated for C₂₁H₂₂FN₄O₄ (M+H)⁺ m/z=413.2; found 413.2.

### Step 7:9-Ethyl-6-fluoro-5-(hydroxymethyl)-2-methyl-2,9-dihydro-3H-pyridazino[3,4,5-de]quinazoline-3,8 (7H)-dione

To a mixture of 9-ethyl-6-fluoro-5-(hydroxymethyl)-7-(4-methoxybenzyl)-2-methyl-2,9-dihydro-3H-pyridazino[3,4,5-de]quinazoline-3,8 (7H)-dione (23 mg, 0.06 mmol) in 2,2,2-trifluoroacetic acid (0.5 mL) was added trifluoromethanesulfonic acid (0.1 mL) at room temperature. The reaction was stirred at room temperature for 1 h, and then was concentrated. The mixture was neutralized with saturated aqueous sodium bicarbonate. The resulting mixture was extracted with ethyl acetate (3×15 mL), dried over anhydrous sodium sulfate. After filtration, the filtrate was concentrated under reduced pressure. The residue was purified by silica gel column chromatography, eluted with 10% methanol in dichloromethane to give the desired product as a yellow solid (15 mg, 92%). LCMS calculated for C₁₃H₁₂FN403 (M−H)⁻ m/z=291.1; found 291.0.

### Step 8: 5-(Chloromethyl)-9-ethyl-6-fluoro-2-methyl-2,9-dihydro-3H-pyridazino[3,4,5-de]quinazoline-3,8 (7H)-dione

To a mixture of 9-ethyl-6-fluoro-5-(hydroxymethyl)-7-(4-methoxybenzyl)-2-methyl-2,9-dihydro-3H-pyridazino[3,4,5-de]quinazoline-3,8 (7H)-dione (13 mg, 0.04 mmol) in dichloromethane (2 mL) was added thionyl chloride (32 mg, 0.27 mmol) followed by one drop of N,N-dimethylformamide. The mixture was stirred at room temperature for 4 h, and then concentrated to give the crude product as a yellow solid (15 mg) which was used directly in the next step without further purification. LCMS calculated for C₁₃H₁₁ClFN₄O₂ (M−H)⁻ m/z=309.1; found 309.0.

### Step 9: N-cyclopropyl-5-(4-((9-ethyl-6-fluoro-2-methyl-3,8-dioxo-2,7,8,9-tetrahydro-3H-pyridazino[3,4,5-de]quinazolin-5-yl)methyl)piperazin-1-yl)-6-methylpicolinamide (I-80)

To a mixture of 5-(chloromethyl)-9-ethyl-6-fluoro-2-methyl-2,9-dihydro-3H-pyridazino[3,4,5-de]quinazoline-3,8 (7H)-dione (15 mg, 0.05 mmol) and N-cyclopropyl-6-methyl-5-(piperazin-1-yl)picolinamide (Example 55, Step 3:25 mg, 0.1 mmol) in acetonitrile (2 mL) was added N-ethyl-N-isopropylpropan-2-amine (125 mg, 0.97 mmol). The mixture was stirred at room temperature for 16 h and then was stirred at 60° C. for 2 h. After cooling to room temperature, the mixture was concentrated. The residue was purified by silica gel column chromatography, eluted with 10% methanol in dichloromethane to give the crude product which was further purified by reversed-phase flash chromatography with the following conditions: column, C18 silica gel; mobile phase, acetonitrile in water (0.1% trifluoroacetic acid), 10% to 40% over 15 min; detector, UV 254 nm. The collected fractions were lyophilized to afford the TFA salt of the desired product as a yellow solid. LCMS calculated for C₂₇H₃₂FN₈O₃ (M+H)⁺ m/z=535.3; found 535.3. ¹H NMR (400 MHz, CD₃OD) δ 7.91 (d, J=8.4 Hz, 1H), 7.56 (d, J=8.4 Hz, 1H), 7.34 (d, J=5.2 Hz, 1H), 4.50 (s, 2H), 3.81 (q, J=7.2 Hz, 2H), 3.63-3.49 (m, 4H), 3.30-3.20 (m, 4H), 3.16 (s, 3H), 2.89-2.82 (m, 1H), 2.57 (s, 3H), 1.27 (t, J=7.2 Hz, 3H), 0.87-0.80 (m, 2H), 0.69-0.63 (m, 2H).

### Example 81: 4-[4-[(6-Ethyl-10-fluoro-7-oxo-2,4,6,8-tetrazatricyclo[7.3.1.05,13]trideca-1,3,5 (13),9,11-pentaen-11-yl)methyl]piperazin-1-yl]-N,3-dimethyl-benzamide (I-81)

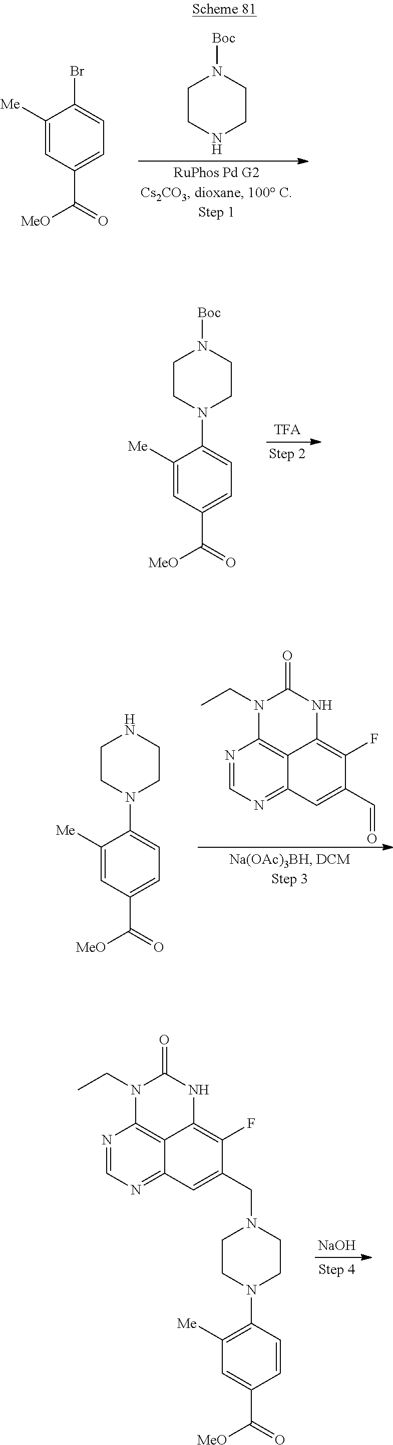

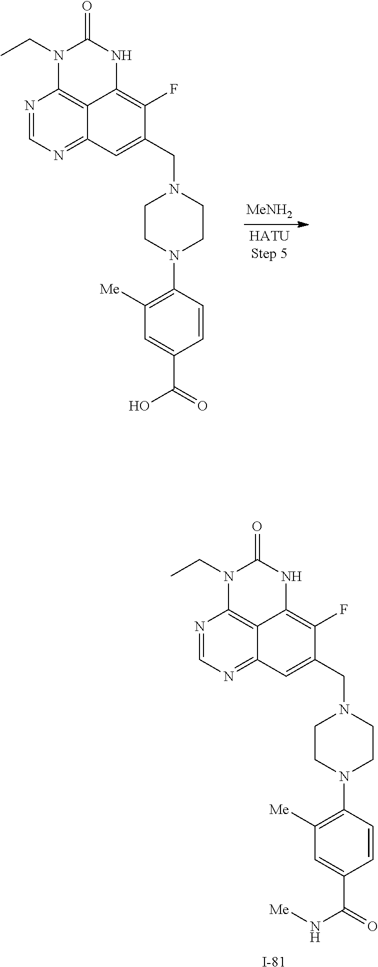

### Step 1: tert-Butyl 4-(4-methoxycarbonyl-2-methyl-phenyl)piperazine-1-carboxylate

To a round bottom flask equipped with a magnetic stir bar was added methyl 4-bromo-3-methyl-benzoate (2.00 g, 8.73 mmol), tert-butyl piperazine-1-carboxylate (1.95 g, 10.48 mmol), [2-(2-aminophenyl)phenyl]-chloro-palladium; dicyclohexyl-[2-(2,6-diisopropoxyphenyl)phenyl]phosphane (1.36 g, 1.75 mmol) and Cs₂CO₃ (8.53 g, 26.19 mmol). The flask was sealed with a rubber septum, evacuated and backfilled nitrogen (this process was repeated a total of three times). 1,4-Dioxane (40 mL) was added. The reaction was stirred at 100° C. for 3 h. Upon cooling to room temperature, the reaction mixture was diluted with water and extracted with CH₂Cl₂ (3×). The combined organic layers were washed with brine, dried with anhydrous Na₂SO₄, filtered, and concentrated. The residue was purified by silica gel column chromatography (40 g, 0-50% EtOAc in Hexane) to provide the desired product (1.89 g, 65%). LCMS calculated for C₁₈H₂₇N₂O₄ (M+H)⁺ m/z=335.2 found 335.2.

### Step 2: Methyl 3-methyl-4-piperazin-1-yl-benzoate

To a stirred solution of tert-butyl 4-(4-methoxycarbonyl-2-methyl-phenyl)piperazine-1-carboxylate (1.80 g, 5.38 mmol) in DCM (15 mL) was added TFA (5 mL). The resulting mixture was stirred at room temperature for 4 h. The reaction mixture was concentrated. To the residue was added sat. NaHCO₃ aq. solution (50 mL), followed by water (50 mL). The precipitate was collected by filtration and dried under vacuum to afford the desired product (1.20 g, 95%). LCMS calculated for C₁₃H₁₉N₂O₂ (M+H)⁺ m/z=235.1; found 235.1.

### Step 3: Methyl 4-(4-((3-ethyl-9-fluoro-2-oxo-2,3-dihydro-1H-pyrimido[4,5,6-de]quinazolin-8-yl)methyl)piperazin-1-yl)-3-methylbenzoate

To a stirred solution of 3-ethyl-9-fluoro-2-oxo-2,3-dihydro-1H-pyrimido[4,5,6-de]quinazoline-8-carbaldehyde (Example 58, Step 3:250 mg, 0.96 mmol) in DCM (5 mL) was added methyl 3-methyl-4-piperazin-1-yl-benzoate (236 mg, 1.01 mmol) followed by triethylamine (145.8 mg, 1.44 mmol). The resulting mixture was stirred at room temperature for 1 h. Then sodium triacetoxyborohydride (488 mg, 2.31 mmol) was added. After stirring at room temperature for 12 h, the reaction was diluted with sat. NaHCO₃ aq. solution (10 mL). The mixture was filtered. The filter cake was rinsed with water and dried under vacuum to afford the desired product (280 mg, 61%). LCMS calculated for C₂₅H₂₈FN₆O₃ (M+H)⁺ m/z=479.2; found 479.1.

### Step 4: 4-(4-((3-Ethyl-9-fluoro-2-oxo-2,3-dihydro-1H-pyrimido[4,5,6-de]quinazolin-8-yl)methyl)piperazin-1-yl)-3-methylbenzoic acid

To a stirred solution of methyl 4-(4-((3-ethyl-9-fluoro-2-oxo-2,3-dihydro-1H-pyrimido[4,5,6-de]quinazolin-8-yl)methyl)piperazin-1-yl)-3-methylbenzoate (250 mg, 0.52 mmol) in THF (2 mL) was added MeOH (1 mL) followed by 2M aqueous NaOH (1.31 mL, 2.62 mmol). The resulting mixture was stirred at 60° C. for 30 min. Upon cooling to room temperature, the reaction mixture was treated with 4N aqueous HCl (1 mL). The precipitate was collected by filtration and dried under vacuum to afford the desired product (172 mg, 71%). LCMS calculated for C₂₄H₂₆FN₆O₃ (M+H)⁺ m/z=465.2; found 465.1.

### Step 5: 4-(4-((3-Ethyl-9-fluoro-2-oxo-2,3-dihydro-1H-pyrimido[4,5,6-de]quinazolin-8-yl)methyl)piperazin-1-yl)-N,3-dimethylbenzamide (I-81)

To a solution of 4-(4-((3-ethyl-9-fluoro-2-oxo-2,3-dihydro-1H-pyrimido[4,5,6-de]quinazolin-8-yl)methyl)piperazin-1-yl)-3-methylbenzoic acid (15.0 mg, 0.03 mmol) in DMF (1 mL) was added [dimethylamino (triazolo[4,5-b]pyridin-3-yloxy)methylene]-dimethyl-ammonium hexafluorophosphate (18.4 mg, 0.05 mmol), N-ethyl-N-isopropyl-propan-2-amine (28.2 uL, 0.16 mmol) and methylamine (2.0 M in THF) (30 uL, 0.06 mmol). After stirring at room temperature for 30 min, the reaction mixture was purified by prep-HPLC (Column: Sunfire prep C18 column; mobile phase A: water (0.05% trifluoroacetic acid), mobile Phase B: acetonitrile; flow rate: 60 mL/min; gradient: 5% B to 30% B over 7 min). Eluted fractions were collected and lyophilized to afford the TFA salt of the desired product as a white solid. LCMS calculated for C₂₅H₂₉FN₇O₂ (M+H)⁺ m/z=478.2; found 478.2.

### Examples 82-84

Examples 82-84 in Table 4 were prepared according to the procedure described in Example 81, using the corresponding amines instead of methylamine.

TABLE 4

| | | LCMS |
|---|---|---|
| | | (M + H)⁺ |
| Example | Chemical Structure & Name | m/z |
| 82 | 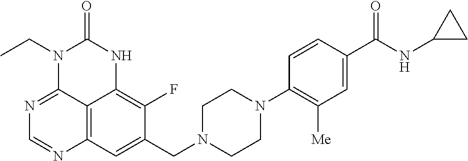 [SMILES: CCN1C(=O)Nc2c(F)c(CN3CCN(c4ccc(C(=O)NC5CC5)cc4C)CC3)cc3ncnc1c23] N-cyclopropyl-4-(4-((3-ethyl-9-fluoro-2-oxo-2,3-dihydro-1H- pyrimido[4,5,6-de]quinazolin-8-yl)methyl)piperazin-1-yl)-3- methylbenzamide | 504.3 |
| 83 | 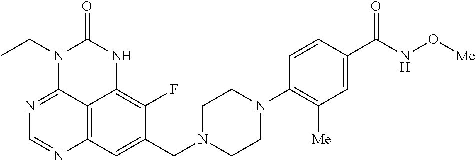 [SMILES: CCN1C(=O)Nc2c(F)c(CN3CCN(c4ccc(C(=O)NOC)cc4C)CC3)cc3ncnc1c23] 4-(4-((3-ethyl-9-fluoro-2-oxo-2,3-dihydro-1H-pyrimido[4,5,6- de]quinazolin-8-yl)methyl)piperazin-1-yl)-N-methoxy-3- methylbenzamide | 494.2 |
| 84 | 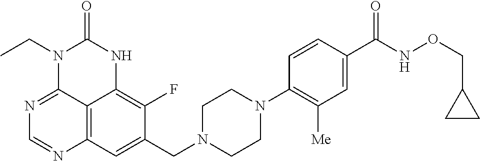 [SMILES: CCN1C(=O)Nc2c(F)c(CN3CCN(c4ccc(C(=O)NOCC5CC5)cc4C)CC3)cc3ncnc1c23] N-(cyclopropylmethoxy)-4-(4-((3-ethyl-9-fluoro-2-oxo-2,3- dihydro-1H-pyrimido[4,5,6-de]quinazolin-8-yl)methyl)piperazin-1- yl)-3-methylbenzamide | 534.3 |

### Example 85: 4-(4-((3-Ethyl-9-fluoro-2-oxo-2,3-dihydro-1H-pyrimido[4,5,6-de]quinazolin-8-yl)methyl)piperazin-1-yl)-3-fluoro-N-methylbenzamide (I-85)

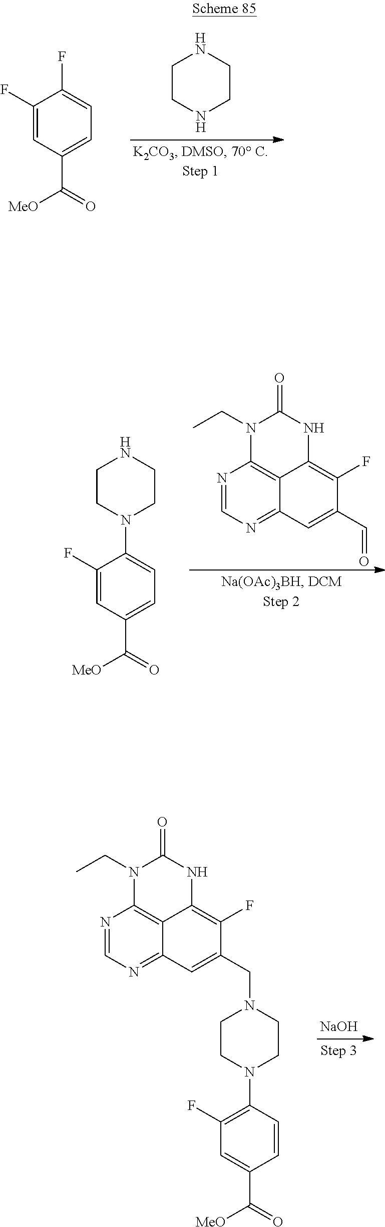

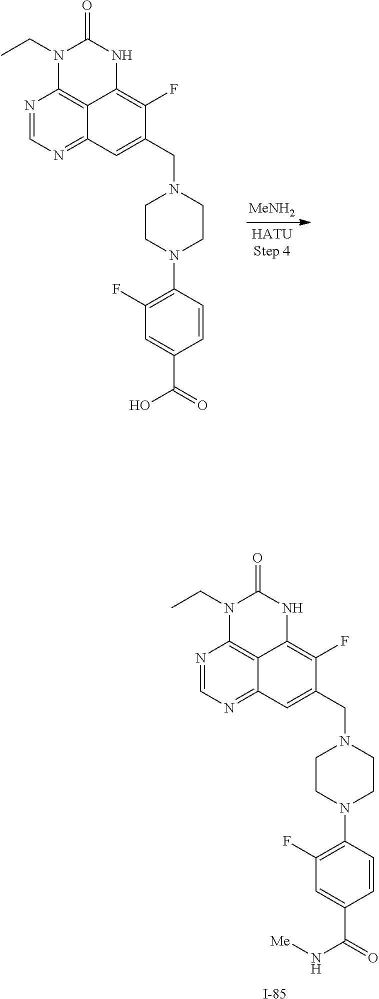

### Step 1: Methyl 3-fluoro-4-(piperazin-1-yl)benzoate

To a stirred solution of methyl 3,4-difluorobenzoate (5.00 g, 29.05 mmol) in DMSO (30 mL) was added piperazine (3.75 g, 43.57 mmol) and K₂CO₃ (10.04 g, 72.62 mmol). The resulting mixture was stirred at 70° C. for 4h. Upon cooling to room temperature, the reaction mixture was poured into water (100 mL). The precipitate was collected by filtration and dried under vacuum to afford the desired product (5.90 g, 85%). LCMS calculated for C₁₂H₁₆FN₂O₂ (M+H)⁺ m/z=239.1; found 239.1.

### Step 2: Methyl 4-(4-((3-ethyl-9-fluoro-2-oxo-2,3-dihydro-1H-pyrimido[4,5,6-de]quinazolin-8-yl)methyl)piperazin-1-yl)-3-fluorobenzoate

To a stirred solution of 3-ethyl-9-fluoro-2-oxo-2,3-dihydro-1H-pyrimido[4,5,6-de]quinazoline-8-carbaldehyde (Example 58, Step 3:250 mg, 0.96 mmol) in DCM (5 mL) was added methyl 3-fluoro-4-piperazin-1-yl-benzoate (251.8 mg, 1.06 mmol) followed by triethylamine (145.8 mg, 1.44 mmol). The resulting mixture was stirred at room temperature for 1 h. Then sodium triacetoxyborohydride (488 mg, 2.31 mmol) was added. After stirring at room temperature for 12 h, the reaction was diluted with sat. NaHCO₃ aq. solution (10 mL). The mixture was filtered. The filter cake was rinsed with water and dried under vacuum to afford the desired product (265 mg, 57%). LCMS calculated for C₂₄H₂₅F₂N₆O₃ (M+H)⁺ m/z=483.2; found 483.1.

### Step 3: 4-(4-((3-Ethyl-9-fluoro-2-oxo-2,3-dihydro-1H-pyrimido[4,5,6-de]quinazolin-8-yl)methyl)piperazin-1-yl)-3-fluorobenzoic acid

To a stirred solution of methyl 4-(4-((3-ethyl-9-fluoro-2-oxo-2,3-dihydro-1H-pyrimido[4,5,6-de]quinazolin-8-yl)methyl)piperazin-1-yl)-3-fluorobenzoate (250 mg, 0.52 mmol) in THF (2 mL) was added MeOH (1 mL) followed by 2M aqueous NaOH (1.31 mL, 2.62 mmol). The resulting mixture was stirred at 60° C. for 30 min. Upon cooling to room temperature, the reaction mixture was treated with 4N aqueous HCl (1 mL). The precipitate was collected by filtration and dried under vacuum to afford the desired product (144 mg, 59%). LCMS calculated for C₂₃H₂₃F₂N₆O₃ (M+H)⁺ m/z=469.2; found 469.1.

### Step 4: 4-(4-((3-Ethyl-9-fluoro-2-oxo-2,3-dihydro-1H-pyrimido[4,5,6-de]quinazolin-8-yl)methyl)piperazin-1-yl)-3-fluoro-N-methylbenzamide (1-85)

To a solution of 4-(4-((3-ethyl-9-fluoro-2-oxo-2,3-dihydro-1H-pyrimido[4,5,6-de]quinazolin-8-yl)methyl)piperazin-1-yl)-3-fluorobenzoic acid (15 mg, 0.03 mmol) in DMF (1 mL) was added [dimethylamino (triazolo[4,5-b]pyridin-3-yloxy)methylene]-dimethyl-ammonium hexafluorophosphate (18.4 mg, 0.05 mmol), N-ethyl-N-isopropyl-propan-2-amine (28.2 uL, 0.16 mmol) and methylamine (2.0 M in THF) (30 uL, 0.06 mmol). After stirring at room temperature for 30 min, the reaction mixture was purified by prep-HPLC (Column: Sunfire prep C₁₈ column; mobile phase A: water (0.05% trifluoroacetic acid), mobile Phase B: acetonitrile; flow rate: 60 mL/min; gradient: 5% B to 30% B over 7 min). Eluted fractions were collected and lyophilized to afford the TFA salt of the desired product as a white solid. LCMS calculated for C₂₄H₂₆F₂N₇O₂ (M+H)⁺ m/z=482.2; found 482.1.

### Examples 86-88

Examples 86-88 in Table 5 were prepared according to the procedure described in Example 85, using the corresponding amines instead of methylamine.

TABLE 5

| | | LCMS |
|---|---|---|
| | | (M + H)⁺ |
| Example | Chemical Structure & Name | m/z |
| 86 | 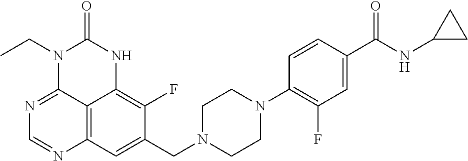 [SMILES: CCN1C(=O)Nc2c(F)c(CN3CCN(c4ccc(C(=O)NC5CC5)cc4F)CC3)cc3ncnc1c23] N-cyclopropyl-4-(4-((3-ethyl-9-fluoro-2-oxo-2,3-dihydro-1H- pyrimido[4,5,6-de]quinazolin-8-yl)methyl)piperazin-1-yl)-3- fluorobenzamide | 508.2 |
| 87 | 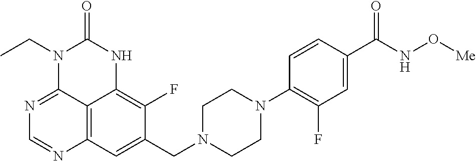 [SMILES: CCN1C(=O)Nc2c(F)c(CN3CCN(c4ccc(C(=O)NOC)cc4F)CC3)cc3ncnc1c23] 4-(4-((3-ethyl-9-fluoro-2-oxo-2,3-dihydro-1H-pyrimido[4,5,6- de]quinazolin-8-yl)methyl)piperazin-1-yl)-3-fluoro-N- methoxybenzamide | 498.2 |
| 88 | 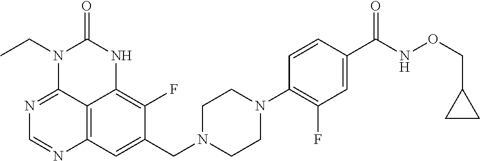 [SMILES: CCN1C(=O)Nc2c(F)c(CN3CCN(c4ccc(C(=O)NOCC5CC5)cc4F)CC3)cc3ncnc1c23] N-(cyclopropylmethoxy)-4-(4-((3-ethyl-9-fluoro-2-oxo-2,3- dihydro-1H-pyrimido[4,5,6-de]quinazolin-8-yl)methyl)piperazin-1- yl)-3-fluorobenzamide | 538.2 |

### Example 89: 4-(4-((3-Ethyl-9-fluoro-2-oxo-2,3-dihydro-1H-pyrimido[4,5,6-de]quinazolin-8-yl)methyl)piperazin-1-yl)-2,3-difluoro-N-methylbenzamide (I-89)

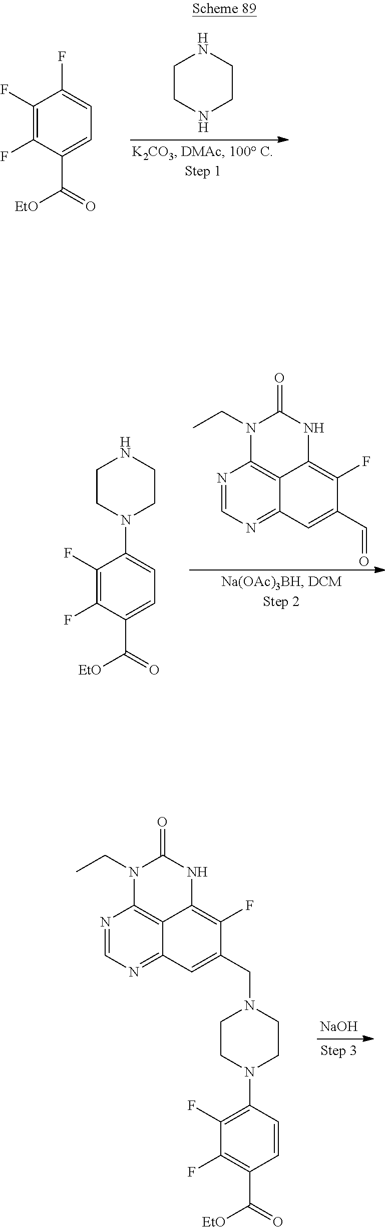

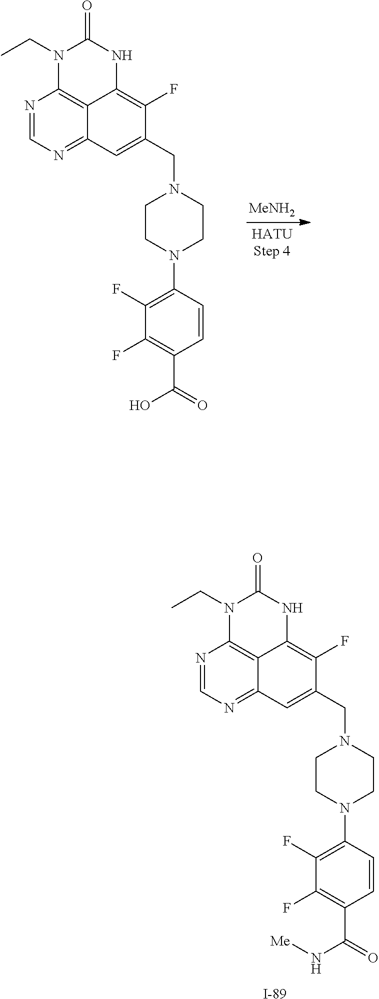

### Step 1: Ethyl 2,3-difluoro-4-(piperazin-1-yl)benzoate

To a stirred solution of ethyl 2,3,4-trifluorobenzoate (1.00 g, 4.90 mmol) in DMAc (10 mL) was added piperazine (1.27 g, 14.70 mmol) and K₂CO₃ (1.69 g, 12.25 mmol). The resulting mixture was stirred at 100° C. for 20 min. Upon cooling to room temperature, the reaction mixture was poured into water (50 mL) and extracted with EtOAc (3×50 mL). The combined organic layers were washed with brine, dried with anhydrous Na₂SO₄, filtered, and concentrated to provide the crude product (1.10 g) which was used directly in the next step without further purification. LCMS calculated for C₁₃H₁₇F₂N₂O₂ (M+H)⁺ m/z=271.1; found 271.1.

### Step 2: Ethyl 4-(4-((3-ethyl-9-fluoro-2-oxo-2,3-dihydro-1H-pyrimido[4,5,6-de]quinazolin-8-yl)methyl)piperazin-1-yl)-2,3-difluorobenzoate

To a stirred solution of 3-ethyl-9-fluoro-2-oxo-2,3-dihydro-1H-pyrimido[4,5,6-de]quinazoline-8-carbaldehyde (Example 58, Step 3:250 mg, 0.96 mmol) in DCM (5 mL) was added ethyl 2,3-difluoro-4-(piperazin-1-yl)benzoate (285.6 mg, 1.06 mmol) followed by triethylamine (145.8 mg, 1.44 mmol). The resulting mixture was stirred at room temperature for 1 h. Then sodium triacetoxyborohydride (488 mg, 2.31 mmol) was added. After stirring at room temperature for 12 h, the reaction was diluted with sat. NaHCO₃ aq. solution (10 mL). The mixture was filtered. The filter cake was rinsed with water and dried under vacuum to afford the desired product (277 mg, 56%). LCMS calculated for C₂₅H₂₆F₃N₆O₃ (M+H)⁺ m/z=515.2; found 515.1

### Step 3: 4-(4-((3-Ethyl-9-fluoro-2-oxo-2,3-dihydro-1H-pyrimido[4,5,6-de]quinazolin-8-yl)methyl)piperazin-1-yl)-2,3-difluorobenzoic acid

To a stirred solution of ethyl 4-(4-((3-ethyl-9-fluoro-2-oxo-2,3-dihydro-1H-pyrimido[4,5,6-de]quinazolin-8-yl)methyl)piperazin-1-yl)-2,3-difluorobenzoate (250 mg, 0.49 mmol) in THF (2 mL) was added MeOH (1 mL) followed by 2M aqueous NaOH (1.21 mL, 2.42 mmol). The resulting mixture was stirred at 60° C. for 30 min. Upon cooling to room temperature, the reaction mixture was treated with 4N aqueous HCl (1 mL). The precipitate was collected by filtration and dried under vacuum to afford the desired product (133 mg). LCMS calculated for C₂₃H₂₂F₃N₆O₃ (M+H)⁺ m/z=487.2; found 487.1.

### Step 4: 4-(4-((3-Ethyl-9-fluoro-2-oxo-2,3-dihydro-1H-pyrimido[4,5,6-de]quinazolin-8-yl)methyl)piperazin-1-yl)-2,3-difluoro-N-methylbenzamide (I-89)

To a solution of 4-(4-((3-ethyl-9-fluoro-2-oxo-2,3-dihydro-1H-pyrimido[4,5,6-de]quinazolin-8-yl)methyl)piperazin-1-yl)-2,3-difluorobenzoic acid (15 mg, 0.03 mmol) in DMF (1 mL) was added [dimethylamino (triazolo[4,5-b]pyridin-3-yloxy)methylene]-dimethyl-ammonium hexafluorophosphate (18.4 mg, 0.05 mmol), N-ethyl-N-isopropyl-propan-2-amine (28.2 uL, 0.16 mmol) and methylamine (2.0 M in THF) (30 uL, 0.06 mmol). After stirring at room temperature for 30 min, the reaction mixture was purified by prep-HPLC (Column: Sunfire prep C18 column; mobile phase A: water (0.05% trifluoroacetic acid), mobile Phase B: acetonitrile; flow rate: 60 mL/min; gradient: 5% B to 30% B over 7 min). Eluted fractions were collected and lyophilized to afford the TFA salt of the desired product as a white solid. LCMS calculated for C₂₄H₂₅F₃N₇O₂ (M+H)⁺ m/z=500.2; found 500.1.

### Examples 90-92

Examples 90-92 in Table 6 were prepared according to the procedure described in Example 89, using the corresponding amines instead of methylamine.

TABLE 6

| | | LCMS |
|---|---|---|
| | | (M + H)⁺ |
| Example | Chemical Structure & Name | m/z |
| 90 | 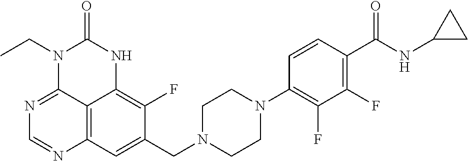 [SMILES: CCN1C(=O)Nc2c(F)c(CN3CCN(c4ccc(C(=O)NC5CC5)c(F)c4F)CC3)cc3ncnc1c23] N-cyclopropyl-4-(4-((3-ethyl-9-fluoro-2-oxo-2,3-dihydro-1H- pyrimido[4,5,6-de]quinazolin-8-yl)methyl)piperazin-1-yl)-2,3- difluorobenzamide | 526.2 |
| 91 | 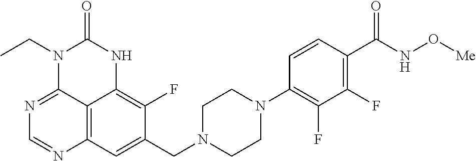 [SMILES: CCN1C(=O)Nc2c(F)c(CN3CCN(c4ccc(C(=O)NOC)c(F)c4F)CC3)cc3ncnc1c23] 4-(4-((3-ethyl-9-fluoro-2-oxo-2,3-dihydro-1H-pyrimido[4,5,6- de]quinazolin-8-yl)methyl)piperazin-1-yl)-2,3-difluoro-N- methoxybenzamide | 516.2 |
| 92 | 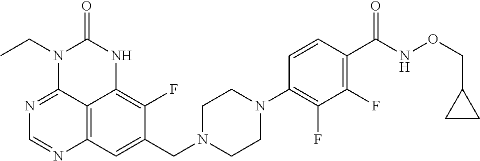 [SMILES: CCN1C(=O)Nc2c(F)c(CN3CCN(c4ccc(C(=O)NOCC5CC5)c(F)c4F)CC3)cc3ncnc1c23] N-(cyclopropylmethoxy)-4-(4-((3-ethyl-9-fluoro-2-oxo-2,3- dihydro-1H-pyrimido[4,5,6-de]quinazolin-8-yl)methyl)piperazin-1- yl)-2,3-difluorobenzamide | 556.2 |

### Example 93: N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-3-methylbenzamide (I-93)

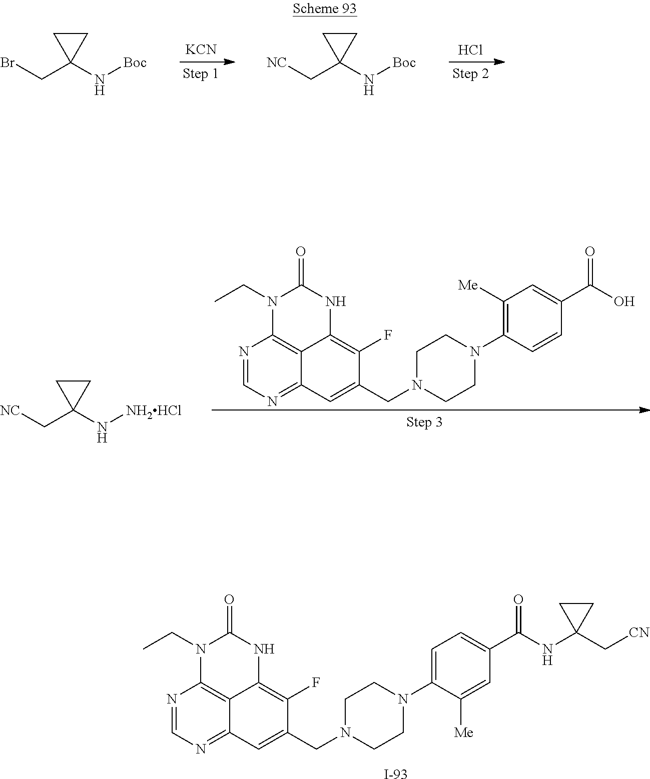

### Step 1: tert-Butyl N-[1-(cyanomethyl)cyclopropyl]carbamate

To a stirred solution of tert-butyl N-[1-(bromomethyl)cyclopropyl]carbamate (500.00 mg, 2.00 mmol) in DMSO (5 mL) was added potassium cyanide (390.49 mg, 6.00 mmol). The resulting mixture was stirred at 60° C. for 3 h. Upon cooling to the room temperature, the reaction was diluted with water and extracted with EtOAc. The organic layer was washed with brine, dried over anhydrous Na₂SO₄, filtered and concentrated to afford the desired product (385 mg). LCMS calculated for C₁₀H₁₇N₂O₂ (M+H)⁺ m/z=197.1; found 197.1.

### Step 2:2-(1-Aminocyclopropyl) acetonitrile hydrochloride

To a stirred solution of tert-butyl N-[1-(cyanomethyl)cyclopropyl]carbamate (150 mg, 0.76 mmol) in DCM (10 mL) was added 4N HCl in dioxane (2.5 mL). The resulting mixture was stirred at room temperature for 30 min. The precipitate was collected by filtration and dried under vacuum to afford the desired product (77 mg, 76%).

### Step 3: N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-3-methylbenzamide (I-93)

To a solution of 4-(4-((3-ethyl-9-fluoro-2-oxo-2,3-dihydro-1H-pyrimido[4,5,6-de]quinazolin-8-yl)methyl)piperazin-1-yl)-3-methylbenzoic acid (Example 81, Step 4:20 mg, 0.04 mmol) in DMF (1 mL) was added [dimethylamino (triazolo[4,5-b]pyridin-3-yloxy)methylene]-dimethyl-ammonium hexafluorophosphate (24.5 mg, 0.06 mmol), N-ethyl-N-isopropyl-propan-2-amine (37.6 uL, 0.22 mmol) and 2-(1-aminocyclopropyl) acetonitrile hydrochloride (11.4 mg, 0.09 mmol). After stirring at room temperature for 30 min, the reaction mixture was purified by prep-HPLC (Column: Sunfire prep C₁₈ column; mobile phase A: water (0.05% trifluoroacetic acid), mobile Phase B: acetonitrile; flow rate: 60 mL/min; gradient: 5% B to 30% B over 7 min). Eluted fractions were collected and lyophilized to afford the TFA salt of the desired product as a white solid. LCMS calculated for C₂₉H₃₂FN₈O₂ (M+H)⁺ m/z=543.3; found 543.3.

### Example 94: N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-3-fluorobenzamide (I-94)

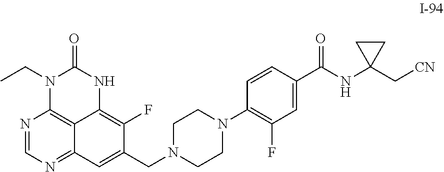 [SMILES: CCN1C(=O)Nc2c(F)c(CN3CCN(c4ccc(C(=O)NC5(CC#N)CC5)cc4F)CC3)cc3ncnc1c23]

Example 94 was prepared according to the procedure described in Example 93, using 4-(4-((3-ethyl-9-fluoro-2-oxo-2,3-dihydro-1H-pyrimido[4,5,6-de]quinazolin-8-yl)methyl)piperazin-1-yl)-3-fluorobenzoic acid (Example 85, Step 3). LCMS calculated for C₂₈H₂₉F₂N₈O₂ (M+H)⁺ m/z=547.2; found 547.2.

### Example 95: N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-2,3-difluorobenzamide (I-95)

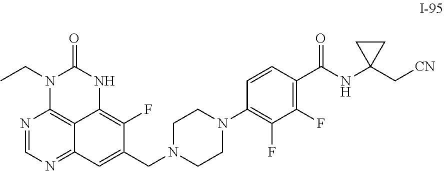 [SMILES: CCN1C(=O)Nc2c(F)c(CN3CCN(c4ccc(C(=O)NC5(CC#N)CC5)c(F)c4F)CC3)cc3ncnc1c23]

Example 95 was prepared according to the procedure described in Example 93, using 4-(4-((3-ethyl-9-fluoro-2-oxo-2,3-dihydro-1H-pyrimido[4,5,6-de]quinazolin-8-yl)methyl)piperazin-1-yl)-2,3-difluorobenzoic acid (Example 89, Step 3). LCMS calculated for C₂₈H₂₈F₃N₈O₂ (M+H)⁺ m/z=565.2; found 565.2.

### Example 96: N-cyclobutyl-5-(4-((3-ethyl-9-fluoro-2-oxo-2,3-dihydro-1H-pyrimido[4,5,6-de]quinazolin-8-yl)methyl)piperazin-1-yl)-6-methylpicolinamide (I-96)

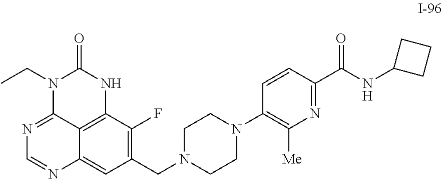 [SMILES: CCN1C(=O)Nc2c(F)c(CN3CCN(c4ccc(C(=O)NC5CCC5)nc4C)CC3)cc3ncnc1c23]

Examples 96 was prepared according to the procedure described in Example 58, using cyclobutylamine hydrochloride instead of O-(cyclopropylmethyl) hydroxylamine hydrochloride. LCMS calculated for C₂₇H₃₂FN₈O₂ (M+H)⁺ m/z=519.3; found 519.3.

### Example A. In-Cell Western Measurement of PARylation in PARP1 WT and Knockout Cells

The purpose of this cell-based assay is to measure the potency and selectivity of synthesized PARP inhibitors in suppressing hydrogen peroxide-induced PARylation in cells with and without PARP1 expression. Several cell lines were used in this assay. The human PARP1 knockout HEK-293T cell line (#ab266598) was purchased from Abcam, and its corresponding wide-type control is the human wild-type HEK-293T cell line (#ab255449). The HAP1 isogenic cell lines with and without the PARP1 gene (#HZGHC003943c006) were purchased from Horizon Discovery. HeLa cells (wide-type with PARP1 gene) were purchased from ATCC. Cells were cultured using complete medium containing 10% fetal bovine serum. One day before the assay, cells were seeded in 96-well plates at the density of 20,000 cells per well in 100 uL complete medium. Compounds were dissolved using DMSO and diluted to the working concentrations using complete medium. Cells were pre-treated with compounds for 30 min and then stimulated with 10 mM hydrogen peroxide for 15 min. After stimulation, cells were immediately fixed using ice-cold methanol for 20 min. After fixation, cells were washed using 1×PBST buffer for 3 times, 5 min each time, to eliminate residual methanol. Cells were blocked for 2 h at room temperature in blocking buffer (LI-COR Biosciences, #927-70001). Primary antibody was diluted in diluent buffer (LI-COR Biosciences, #927-75001, 1:200) at 50 uL per well. Cells were incubated with the primary antibody at 4° C. for 18 h. The wells were then washed using 1×PBST for 3 times, 5 min each time. IRDye® 680RD Goat anti-Mouse IgG (H+L) secondary antibody was diluted at 1/2000 using the diluent buffer and added at 100 μL per well. Secondary antibody incubation was for 1 h at room temperature. Cells were then washed using 1×PBST for 3 times, 5 min each time. The signal was detected and analyzed using LI-COR Odyssey DLx Imaging system. Data were collected and further processed using GraphPad Prism for IC₅₀ estimation.

Results of the assay described above for HeLa_WT are presented in Table 2. Compounds denoted of the present disclosure showed IC₅₀ values in the following ranges:
A: IC₅₀≤10 nM;
B: 10 nM<IC₅₀≤100 nM;
C: 100 nM<IC₅₀≤500 nM;
D: 500 nM<IC₅₀≤1,000 nM;
E: 1,000 nM<IC₅₀≤10,000 nM.

TABLE 2

| Compound | HeLa_WT_IC₅₀ (nM) |
|---|---|
| I-1 | A |
| I-2 | B |
| I-3 | C |
| I-4 | A |
| I-5 | B |
| I-6 | A |
| I-7 | A |
| I-8 | A |
| I-9 | C |
| I-10 | B |
| I-11 | B |
| I-12 | A |
| I-13 | A |
| I-14 | A |
| I-15 | A |
| I-16 | A |
| I-17 | A |
| I-18 | A |
| I-19 | A |
| I-20 | A |
| I-21 | A |
| I-22 | A |
| I-23 | A |
| I-24 | A |
| I-25 | A |
| I-26 | A |
| I-27 | A |
| I-28 | A |
| I-29 | A |
| I-30 | A |
| I-31 | A |
| I-32 | A |
| I-33 | A |
| I-34 | A |
| I-35 | A |
| I-36 | A |
| I-37 | A |
| I-38 | A |
| I-39 | A |
| I-40 | A |
| I-41 | A |
| I-42 | A |
| I-43 | A |
| I-44 | A |
| I-45 | A |
| I-46 | A |
| I-47 | A |
| I-48 | A |
| I-49 | A |
| I-50 | A |
| I-51 | B |
| I-52 | A |
| I-54 | A |
| I-55 | A |
| I-56 | A |
| I-57 | A |
| I-58 | A |
| I-59 | A |
| I-60 | A |
| I-62 | A |
| I-63 | A |
| I-65 | A |
| I-66 | A |
| I-67 | A |
| I-69 | B |
| I-70 | A |
| I-71 | A |
| I-73 | A |
| I-74 | B |
| I-75 | B |
| I-77 | B |
| I-78 | A |
| I-79 | A |
| I-80 | B |
| I-82 | A |
| I-86 | B |
| I-90 | A |
| I-93 | A |
| I-94 | A |
| I-95 | B |
| I-96 | A |

### Example B. High Content Imaging Measurement of PARylation in Cells with and without the PARP1 Gene

The purpose of this cellular assay is to measure inhibitory activity of PARP inhibitors on hydrogen peroxide-induced PARylation in cells with and without the PARP1 gene. Multiple cell lines with different genetic background were tested in this assay. The protocol described below is for Hela cells. Compounds were dissolved in DMSO and kept at −20° C. for long term storage. The assay was performed using 96-well plates with 10,000-20,000 cells per well. Cells were cultured overnight before compound treatment and hydrogen peroxide stimulation. Compounds were diluted using complete medium to final concentrations and added to cells 30 min before hydrogen peroxide stimulation. PARylation signal was induced using hydrogen peroxide at a final concentration of 10 mM for 15 min. After signal induction, cells were immediately fixed using ice-cold methanol for 20 min. These cells were then washed 3 times using 1×PBST buffer, 5 min each time, to eliminate residual methanol. Plates were then blocked for 2 h in blocking buffer (5% goat serum and 0.1% Triton X-100 in PBST). Primary antibody (clone 10H from Trevigen) was diluted to a final concentration of 5 μg/ml using blocking buffer. Primary antibodies were added at 50 uL per well. The plates were kept at 4° C. for 18 h without shaking. Cells were then washed using 1×PBST buffer for 3 times, 5 min each. Goat anti-mouse IgG (H+L), F(ab′)2 Fragment (Alexa Fluor® 488 Conjugate) secondary antibodies were purchased from Cell Signaling Technology. Secondary antibodies were diluted at 1/2000 using the blocking buffer and added at 50 uL per well. The secondary antibody incubation was for 1 h at room temperature with gentle orbital shaking. Cells were then washed using 1×PBST buffer for 3 times, 5 min each. Nucleus staining was performed using DAPI. Fluorescence images were collected using the PICO imaging system with 10× objective lens. Images were analyzed using the CellReporterXpress Image Acquisition and Analysis Software purchased from Molecular Devices. Data were transferred and further processed using GraphPad Prism for IC50 estimation.

### Example C. Cell-Titer Glo Measurement of Cytotoxicity in BRCA2 Isogenic Cells

The purpose of this cellular assay is to measure cytotoxicity and cell killing activity of selected PARP1 inhibitors in DLD1 isogenic cells with and without the BRCA2 genes. The BRCA2 knockout DLD1 cell line (#HD 105-007) and its isogenic wide-type control were purchased from Horizon Discovery. Cells were cultured using RPMI 1640 complete medium containing 10% fetal bovine serum and Penicillin-Streptomycin. One day before the assay, cells were seeded at 50-300 cells per well in 96-well plates. Compounds were dissolved in DMSO and diluted into complete medium to working concentrations and added to each well. The treatment was for 7 days. At the end of this assay, Promega Cell-titer Glo reagents (#G7573) were added at 100 μL per well. Plates were kept on orbital shaker for 2 min. These plates were then kept in the CO2 incubator for another 10 minutes. Luminescence signal was measured using the i3× plate reader. Data were further analyzed using GraphPad Prism for IC50 estimation.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula I: 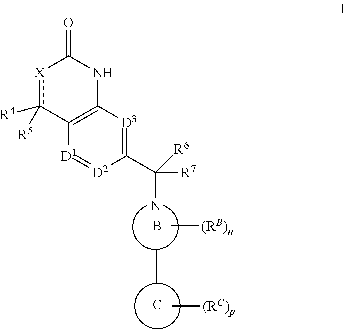
or a pharmaceutically acceptable salt thereof, wherein: [symbol]
is a single or double bond;
X is —C(R¹)═, —C(R¹R²)—, or —N(R^{a})—, as valency allows;
when X is —C(R¹)—, then one of (i)-(iii) applies:
(i)R⁵ is absent;
R¹ and R⁴ are taken together with the carbon atoms to which they are attached to form 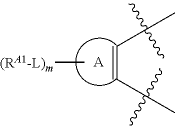
fused to the depicted lactam ring, wherein
Ring A is 5-membered partially unsaturated monocyclic carbocyclyl or 5-membered monocyclic heteroaryl having 1-4 heteroatoms independently selected from nitrogen, oxygen, and sulfur;
(ii)R⁵ is absent;
R⁴ and L^{D1}-R⁸ are taken together with the carbon atoms to which they are attached to form an optionally substituted ring selected from 5- to 7-membered partially unsaturated carbocyclyl or 5- to 7-membered partially unsaturated monocyclic heterocyclyl having 1-3 heteroatoms independently selected from nitrogen, oxygen, and sulfur; or
(iii)R⁵ is absent;
D¹ is S or NR, and D² is absent;
when X is —C(R¹R²)— or —N(R^{a})—:
R¹ and R² are each independently hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or
R¹ and R² are taken together with the carbon atom to which they are attached to form an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur; or
R² and R⁴ are taken together with the carbon atoms to which they are attached to form 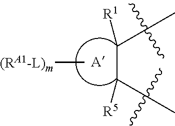
fused to the depicted lactam ring, wherein
Ring A′ is 3- to 7-membered saturated or partially unsaturated monocyclic carbocyclyl or 3- to 7-membered saturated or partially unsaturated monocyclic heterocyclyl having 1-3 heteroatoms independently selected from nitrogen, oxygen, and sulfur;
R^{a} is hydrogen or —L^{R3}-R³,
L^{R3} is a covalent bond or optionally substituted bivalent C₁₋₆ aliphatic;
R³ is hydrogen or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
R⁴ and R⁵ are each independently hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or
R⁴ and R⁵ are taken together with the carbon atom *C to which they are attached to form *C═O, *C═S, *C═NRL, or an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur; or
R⁵ is absent; R⁴ and L^{D1}-R⁸ are taken together with the carbon to which they are attached to form an optionally substituted ring selected from phenyl, 5- to 6-membered monocyclic heteroaryl having 1-4 heteroatoms independently selected from nitrogen, oxygen, and sulfur;
R^{L} is hydrogen, —CN, —OR^{L1}, or optionally substituted C₁₋₆ alkyl;
R^{L1} is hydrogen, C₁₋₆ alkyl, or C₁₋₆ haloalkyl;
each L is independently a covalent bond or optionally substituted bivalent C₁₋₆ aliphatic;
each R^{A1} is independently halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
R⁶ and R⁷ are each independently hydrogen, halogen, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur; or
R⁶ and R⁷ are taken together with the carbon to which they are attached to form an optionally substituted ring selected from 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur;
D¹ is C-L^{D1}-R⁸, N, NR, or S;
D² is absent, C-L^{D2}-R⁹, or N, wherein when D¹ is S or NR, D² is absent;
D³ is CR¹⁰ or N;
L^{D1} is a covalent bond or optionally substituted bivalent C₁₋₆ aliphatic;
L^{D2} is a covalent bond or optionally substituted bivalent C₁₋₆ aliphatic;
R⁸ is hydrogen, halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur;
R⁹ and R¹⁰ are each independently hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, or an optionally substituted group selected from C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur;
Ring B is 3- to 7-membered saturated or partially unsaturated monocyclic heterocyclylene having 1-3 heteroatoms independently selected from nitrogen, oxygen, and sulfur, 6- to 10-membered saturated or partially unsaturated bicyclic heterocyclylene having 1-4 heteroatoms independently selected from nitrogen, oxygen, and sulfur, or 9- to 16-membered saturated or partially unsaturated polycyclic heterocyclylene having 1-5 heteroatoms independently selected from nitrogen, oxygen, and sulfur;
Ring C is phenyl, 8- to 10-membered bicyclic aryl, 10- to 14-membered polycyclic aryl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 10- to 16-membered polycyclic heteroaryl having 1-5 heteroatoms independently selected from nitrogen, oxygen, and sulfur, or 6- to 10-membered saturated or partially unsaturated bicyclic heterocyclyl having 1-4 heteroatoms independently selected from nitrogen, oxygen, and sulfur;
each R^{B} is independently —L^{RB}-R¹¹;
each L^{RB} is independently a covalent bond or optionally substituted bivalent C₁₋₆ aliphatic;
each R^{C} is independently -L^{RC}-R¹²;
each L^{RC} is independently a covalent bond or optionally substituted bivalent C₁₋₆ aliphatic;
R¹¹ and R¹² are each independently halogen, ═O, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
a R^{B} and a R^{C} are taken together with their intervening atoms to form Ring D fused with one or both of Ring B and Ring C, wherein
Ring D is an optionally substituted ring selected from 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 6- to 8-membered saturated or partially unsaturated bicyclic heterocyclyl having 1-4 heteroatoms independently selected from nitrogen, oxygen, and sulfur, phenyl, and 5- to 6-membered monocyclic heteroaryl having 1-4 heteroatoms independently selected from nitrogen, oxygen, and sulfur;
each R is independently hydrogen or an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
two R when attached to the same nitrogen atom are taken together to form optionally substituted 3- to 7-membered saturated or partially unsaturated monocyclic heterocyclyl having 0-2 additional heteroatoms independently selected from nitrogen, oxygen, and sulfur;
each R′ is independently an optionally substituted group selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur; or
two R′ when attached to the same nitrogen atom are taken together to form optionally substituted 3- to 7-membered saturated or partially unsaturated monocyclic heterocyclyl having 0-2 additional heteroatoms independently selected from nitrogen, oxygen, and sulfur;
each R^{m} is independently —OH, —CN, or R;
m is 0, 1, 2, 3, or 4;
n is 0, 1, 2, 3, or 4; and
p is 0, 1, 2, 3, 4, or 5.

2. The compound of claim 1, or a pharmaceutically acceptable salt thereof, wherein: [symbol]
is a single or double bond;
X is —C(R¹)═, —C(R¹R²)—, or —N(R^{a})—, as valency allows;
when X is —C(R¹)═, then one of (i)-(iii) applies:
(i)R⁵ is absent;
R¹ and R⁴ are taken together with the carbon atoms to which they are attached to form 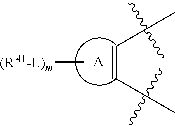
fused to the depicted lactam ring, wherein
Ring A is 5-membered partially unsaturated monocyclic carbocyclyl or 5-membered monocyclic heteroaryl having 1-4 heteroatoms independently selected from nitrogen, oxygen, and sulfur;
(ii)R⁵ is absent;
R⁴ and L^{D1}-R⁸ are taken together with the carbon atoms to which they are attached to form a 5- to 7-membered partially unsaturated carbocyclyl or 5- to 7-membered partially unsaturated monocyclic heterocyclyl having 1-3 heteroatoms independently selected from nitrogen, oxygen, and sulfur, wherein the 5- to 7-membered partially unsaturated carbocyclyl or 5- to 7-membered partially unsaturated monocyclic heterocyclyl are each optionally substituted by 1, 2, 3, or 4 independently selected R^{4A} substituents; or
(iii)R⁵ is absent;
D¹ is S or NR, and D² is absent;
when X is —C(R¹R²)— or —N(R^{a})—:
R¹ and R² are each independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{1A} substituents; or
R¹ and R² are taken together with the carbon atom to which they are attached to form a 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl having 1-3 heteroatoms independently selected from nitrogen, oxygen, and sulfur, or a 6- to 10-membered saturated or partially unsaturated bicyclic heterocyclyl having 1-4 heteroatoms independently selected from nitrogen, oxygen, and sulfur, wherein the 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl, and 6- to 10-membered saturated or partially unsaturated bicyclic heterocyclyl are each optionally substituted with 1, 2, 3, or 4 independently selected R^{1A} substituents; or
R² and R⁴ are taken together with the carbon atoms to which they are attached to form 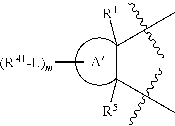
fused to the depicted lactam ring, wherein
Ring A′ is 3- to 7-membered saturated or partially unsaturated monocyclic carbocyclyl or 3- to 7-membered saturated or partially unsaturated monocyclic heterocyclyl having 1-3 heteroatoms independently selected from nitrogen, oxygen, and sulfur;
R^{a} is hydrogen or —L^{R3}-R³,
L^{R3} is a covalent bond or a bivalent C₁₋₆ aliphatic, wherein the bivalent C₁₋₆ aliphatic is optionally substituted with 1, 2, 3, or 4 independently selected RN substituents;
R³ is selected from hydrogen, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{3A} substituents;
R⁴ and R⁵ are each independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{4A} substituents; or
R⁴ and R⁵ are taken together with the carbon atom *C to which they are attached to form *C—O, *C═S, *C═NR^{L}, a 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl having 1-3 heteroatoms independently selected from nitrogen, oxygen, and sulfur, or 6- to 10-membered saturated or partially unsaturated bicyclic heterocyclyl having 1-4 heteroatoms independently selected from nitrogen, oxygen, and sulfur, wherein the 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl, and 6- to 10-membered saturated or partially unsaturated bicyclic heterocyclyl are each optionally substituted with 1, 2, 3, or 4 independently selected R^{4A} substituents; or
R⁵ is absent, and R⁴ and L^{D1}-R⁸, taken together with the carbon to which they are attached, form a group selected from phenyl and 5- to 6-membered monocyclic heteroaryl having 1-4 heteroatoms independently selected from nitrogen, oxygen, and sulfur, wherein the phenyl and 5- to 6-membered monocyclic heteroaryl having 1-4 heteroatoms independently selected from nitrogen, oxygen, and sulfur are each optionally substituted with 1, 2, 3, or 4 independently selected R^{4A} substituents;
R^{L} is hydrogen, —CN, —OR^{L1}, or C₁₋₆ alkyl, wherein the C₁₋₆ alkyl is optionally substituted with 1, 2, 3, or 4 independently selected RN substituents;
R^{L1} is hydrogen, C₁₋₆ alkyl, or C₁₋₆ haloalkyl;
each L is independently a covalent bond or a bivalent C₁₋₆ aliphatic, wherein the bivalent C₁₋₆ aliphatic is optionally substituted with 1, 2, 3, or 4 independently selected RN substituents;
each R^{A1} is independently selected from halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{B1} substituents;
R⁶ and R⁷ are each independently hydrogen, halogen, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{6A} substituents; or
R⁶ and R⁷ are taken together with the carbon to which they are attached to form a 3- to 7-membered saturated or partially unsaturated monocyclic carbocyclyl, a 6- to 10-membered saturated or partially unsaturated bicyclic carbocyclyl, a 3- to 7-membered saturated or partially unsaturated monocyclic heterocyclyl having 1-3 heteroatoms independently selected from nitrogen, oxygen, and sulfur, or a 6- to 10-membered saturated or partially unsaturated bicyclic heterocyclyl having 1-4 heteroatoms independently selected from nitrogen, oxygen, and sulfur, wherein the 3- to 7-membered saturated or partially unsaturated monocyclic carbocyclyl, 6- to 10-membered saturated or partially unsaturated bicyclic carbocyclyl, 3- to 7-membered saturated or partially unsaturated monocyclic heterocyclyl, and 6- to 10-membered saturated or partially unsaturated bicyclic heterocyclyl are each optionally substituted with 1, 2, 3, or 4 independently selected R^{6A} substituents;
D¹ is C-L^{D1}-R⁸, N, NR, or S;
D² is absent, C-L^{D2}-R⁹, or N, wherein when D¹ is S or NR, D² is absent;
D³ is CR¹⁰ or N;
L^{D1} is a covalent bond or a bivalent C₁₋₆ aliphatic, wherein the bivalent C₁₋₆ aliphatic is optionally substituted with 1, 2, 3, or 4 independently selected RN substituents;
L^{D2} is a covalent bond or a bivalent C₁₋₆ aliphatic, wherein the bivalent C₁₋₆ aliphatic is optionally substituted with 1, 2, 3, or 4 independently selected RN substituents;
R⁸ is selected from hydrogen, halogen, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{8A} substituents;
R⁹ and R¹⁰ are each independently selected from hydrogen, halogen, —CN, —OR, —SR, —N(R)₂, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —NRS(O)N(R)₂, —NRS(O)R′, —NRS(O)₂N(R)₂, —S(O)N(R)₂, C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, and 6- to 8-membered saturated or partially unsaturated bicyclic heterocyclyl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, and 6- to 8-membered saturated or partially unsaturated bicyclic heterocyclyl are each optionally substituted with 1, 2, 3, or 4 independently selected R^{9A} substituents;
Ring B is 3- to 7-membered saturated or partially unsaturated monocyclic heterocyclylene having 1-3 heteroatoms independently selected from nitrogen, oxygen, and sulfur, 6- to 10-membered saturated or partially unsaturated bicyclic heterocyclylene having 1-4 heteroatoms independently selected from nitrogen, oxygen, and sulfur, or 9- to 16-membered saturated or partially unsaturated polycyclic heterocyclylene having 1-5 heteroatoms independently selected from nitrogen, oxygen, and sulfur;
Ring C is phenyl, 8- to 10-membered bicyclic aryl, 10- to 14-membered polycyclic aryl, 5- to 6-membered monocyclic heteroaryl having 1-4 heteroatoms independently selected from nitrogen, oxygen, and sulfur, 8- to 10-membered bicyclic heteroaryl having 1-4 heteroatoms independently selected from nitrogen, oxygen, and sulfur, 10- to 16-membered polycyclic heteroaryl having 1-5 heteroatoms independently selected from nitrogen, oxygen, and sulfur, or 6- to 10-membered saturated or partially unsaturated bicyclic heterocyclyl having 1-4 heteroatoms independently selected from nitrogen, oxygen, and sulfur;
each R^{B} is independently —L^{RB}-R¹¹;
each L^{RB} is independently a covalent bond or a bivalent C₁₋₆ aliphatic, wherein the bivalent C₁₋₆ aliphatic is optionally substituted with 1, 2, 3, or 4 independently selected RN substituents;
each R^{C} is independently —L^{RC}-R¹²;
each L^{RC} is independently a covalent bond or a bivalent C₁₋₆ aliphatic, wherein the bivalent C₁₋₆ aliphatic is optionally substituted with 1, 2, 3, or 4 independently selected RN substituents;
R¹¹ and R¹² are each independently selected from halogen, ═O, —CN, —OR, —SR, —N(R)₂, —N⁺(R)₃, —NO₂, —C(O)R′, —C(O)OR, —C(O)N(R)₂, —OC(O)R′, —OC(O)N(R)₂, —OC(O)OR, —OSO₂R′, —OSO₂N(R)₂, —N(R)C(O)R′, —N(R)SO₂R′, —S(O)R′, —SO₂R′, —SO₂N(R)₂, —SO₃R′, —NHOR, —C(O)NR(OR), —NRC(O)OR, —NRC(O)N(R)₂, —C(═NR^{m})R′, —C(═NR^{m})N(R)₂, —NRC(═NR^{m})N(R)₂, —NRC(═NR^{m})R′, —NRS(O)N(R)₂, —NRS(O)R′, —NRS(O)(═NR^{m})R′, —NRS(O)₂N(R)₂, —S(O)N(R)₂, —OS(O)(═R^{m})R′, —S(O)(═NR^{m})R′, —P(O)(R)₂, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected R^{11A} substituents; or
a R^{B} and a R^{C} are taken together with their intervening atoms to form Ring D fused with one or both of Ring B and Ring C, wherein
Ring D is selected from 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl having 1-3 heteroatoms independently selected from nitrogen, oxygen, and sulfur, 6- to 8-membered saturated or partially unsaturated bicyclic heterocyclyl having 1-4 heteroatoms independently selected from nitrogen, oxygen, and sulfur, phenyl, and 5- to 6-membered monocyclic heteroaryl having 1-4 heteroatoms independently selected from nitrogen, oxygen, and sulfur, wherein the 3- to 7-membered saturated or partially unsaturated monocyclic carbocyclyl, 6- to 8-membered saturated or partially unsaturated bicyclic carbocyclyl, 3- to 7-membered saturated or partially unsaturated monocyclic heterocyclyl, 6- to 8-membered saturated or partially unsaturated bicyclic heterocyclyl, phenyl, and 5- to 6-membered monocyclic heteroaryl are each optionally substituted with 1, 2, 3, or 4 independently selected R^{D1} substituents;
each R is independently selected from hydrogen, C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected RN substituents; or
two R when attached to the same nitrogen atom are taken together to form a 3- to 7-membered saturated or partially unsaturated monocyclic heterocyclyl having 0-2 additional heteroatoms independently selected from nitrogen, oxygen, and sulfur, wherein the 3- to 7-membered saturated or partially unsaturated monocyclic heterocyclyl is optionally substituted with 1, 2, 3, or 4 independently selected RN substituents;
each R′ is independently selected from C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl having 1-3 heteroatoms independently selected from nitrogen, oxygen, and sulfur, 6- to 10-membered saturated or partially unsaturated bicyclic heterocyclyl having 1-4 heteroatoms independently selected from nitrogen, oxygen, and sulfur, 5- to 6-membered monocyclic heteroaryl having 1-4 heteroatoms independently selected from nitrogen, oxygen, and sulfur, and 8- to 10-membered bicyclic heteroaryl having 1-4 heteroatoms independently selected from nitrogen, oxygen, and sulfur, wherein the C₁₋₆ aliphatic, 3- to 7-membered saturated or partially unsaturated monocyclic carbocyclyl, 6- to 10-membered saturated or partially unsaturated bicyclic carbocyclyl, phenyl, 8- to 10-membered bicyclic aryl, 3- to 7-membered saturated or partially unsaturated monocyclic heterocyclyl, 6- to 10-membered saturated or partially unsaturated bicyclic heterocyclyl, 5- to 6-membered monocyclic heteroaryl, and 8- to 10-membered bicyclic heteroaryl are each optionally substituted with 1, 2, 3, or 4 independently selected RN substituents; or
two R′ when attached to the same nitrogen atom are taken together to a 3- to 7-membered saturated or partially unsaturated monocyclic heterocyclyl having 0-2 additional heteroatoms independently selected from nitrogen, oxygen, and sulfur, wherein the 3- to 7-membered saturated or partially unsaturated monocyclic heterocyclyl is optionally substituted with 1, 2, 3, or 4 independently selected RN substituents;
each R^{1A}, R^{3A}, R^{4A}, R^{6A}, R^{8A}, R^{9A}, R^{11A}, R^{B1}, R^{D1} and R^{N} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH—CHPh, —(CH₂)₀₋₄O(CH₂)₀₋₁-pyridyl, —NO₂, —CN, —N³, —(CH₂)₀₋₄N(R^{∘})₂, —(CH₂)₀₋₄N(R^{∘})C(O)R^{∘}, —N(R^{∘})C(S)R^{∘}, —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene)C(O)O—N(R^{∘})₂;
each R^{∘} is independently hydrogen, C₁₋₆ aliphatic, —CH₂Ph, —O(CH₂)₀₋₁Ph, —CH₂ (5- to 6-membered heteroaryl ring), or a 3- to 6-membered saturated, partially unsaturated, or aryl ring having 0-4 heteroatoms independently selected from nitrogen, oxygen, or sulfur;
or two independent occurrences of R^{∘}, taken together with their intervening atoms, form a 3- to 12-membered saturated, partially unsaturated, or aryl mono- or bicyclic ring having 0-4 heteroatoms independently selected from nitrogen, oxygen, or sulfur;
each R^{m} is independently —OH, —CN, or R;
m is 0, 1, 2, 3, or 4;
n is 0, 1, 2, 3, or 4; and
p is 0, 1, 2, 3, 4, or 5.

3. The compound of claim 1, wherein the compound is of Formula VIII: 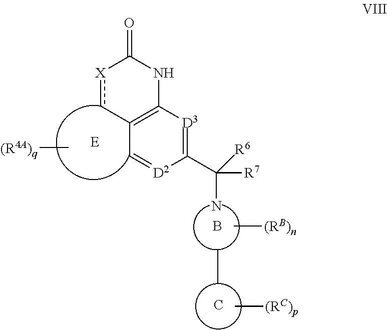
or a pharmaceutically acceptable salt thereof, wherein:
when X is —C(R¹)═, Ring E is selected from 5- to 7-membered partially unsaturated carbocyclyl and 5- to 7-membered partially unsaturated monocyclic heterocyclyl having 1-3 heteroatoms independently selected from nitrogen, oxygen, and sulfur;
when X is —C(R¹R²)— or —N(R^{a})—, Ring E is selected from phenyl and 5- to 6-membered monocyclic heteroaryl having 1-4 heteroatoms independently selected from nitrogen, oxygen, and sulfur;
q is 0, 1, 2, 3, or 4;
each R^{4A} is independently selected from halogen, —(CH₂)₀₋₄R^{∘}, —(CH₂)₀₋₄OR^{∘}, —O(CH₂)₀₋₄R^{∘}, —O—(CH₂)₀₋₄C(O)OR^{∘}, —(CH₂)₀₋₄CH(OR^{∘})₂, —(CH₂)₀₋₄SR^{∘}, —(CH₂)₀₋₄Ph, —(CH₂)₀₋₄O(CH₂)₀₋₁Ph, —CH═CHPh, —(CH₂)₀₋₄O(CH₂)₀₋₁-pyridyl, —NO₂, —CN, —N₃, —(CH₂)₀₋₄N(R^{∘})₂, —(CH₂)₀₋₄N(R^{∘})C(O)R^{∘}), —N(R^{∘})C(S)R^{∘}), —(CH₂)₀₋₄N(R^{∘})C(O)NR^{∘}₂, —N(R^{∘})C(S)NR^{∘}₂, —(CH₂)₀₋₄N(R^{∘})C(O)OR^{∘}, —N(R^{∘})N(R^{∘})C(O)R^{∘}, —N(R^{∘})N(R^{∘})C(O)NR^{∘}₂, —N(R^{∘})N(R^{∘})C(O)OR^{∘}, —(CH₂)₀₋₄C(O)R^{∘}, —C(S)R^{∘}, —(CH₂)₀₋₄C(O)OR^{∘}, —(CH₂)₀₋₄C(O)SR^{∘}, —(CH₂)₀₋₄C(O)OSiR^{∘}₃, —(CH₂)₀₋₄OC(O)R^{∘}, —OC(O)(CH₂)₀₋₄SR^{∘}, —(CH₂)₀₋₄SC(O)R^{∘}, —(CH₂)₀₋₄C(O)NR^{∘}₂, —C(S)NR^{∘}₂, —C(S)SR^{∘}, —SC(S)SR^{∘}, —(CH₂)₀₋₄OC(O)NR^{∘}₂, —C(O)N(OR^{∘})R^{∘}, —C(O)C(O)R^{∘}, —C(O)CH₂C(O)R^{∘}, —C(NOR^{∘})R^{∘}, —(CH₂)₀₋₄SSR^{∘}, —(CH₂)₀₋₄S(O)₂R^{∘}, —(CH₂)₀₋₄S(O)(═NR^{∘})R^{∘}, —(CH₂)₀₋₄S(O)₂OR^{∘}, —(CH₂)₀₋₄OS(O)₂R^{∘}, —(CH₂)₀₋₄—S(O)₂NR^{∘}₂, —(CH₂)₀₋₄S(O)(═NR^{∘})NR^{∘}₂, —(CH₂)₀₋₄S(O)R^{∘}, —N(R^{∘})S(O)₂NR^{∘}₂, —N(R^{∘})S(O)₂R^{∘}, —N(R^{∘})S(O)(═NR^{∘})R^{∘}, —N(OR^{∘})R^{∘}, —C(NH)NR^{∘}₂, —P(O)₂R^{∘}, —P(O)R^{∘}₂, —OP(O)R^{∘}₂, —OP(O)(OR^{∘})₂, —SiR^{∘}₃, —(C₁₋₄ straight or branched) alkylene)O—N(R^{∘})₂, and —(C₁₋₄ straight or branched) alkylene) C(O)O—N(R^{∘})₂; and
each R^{∘} is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur;
or two independent occurrences of R^{∘}, taken together with their intervening atoms, form a 3- to 12-membered saturated, partially unsaturated, or aryl mono- or bicyclic ring having 0-4 heteroatoms independently selected from nitrogen, oxygen, or sulfur.

4. The compound of claim 1, or a pharmaceutically acceptable salt thereof, wherein X is —N(R^{a})—.

5. The compound of claim 1, or a pharmaceutically acceptable salt thereof, wherein R^{a} is —L^{R3}-R³.

6. The compound of claim 1, or a pharmaceutically acceptable salt thereof, wherein L^{R3} is a covalent bond.

7. The compound of claim 1, or a pharmaceutically acceptable salt thereof, wherein R³ is hydrogen or optionally substituted C₁₋₆ aliphatic.

8. The compound of claim 1, or a pharmaceutically acceptable salt thereof, wherein R^{a} is —CH₂CH₃.

9. The compound of claim 3, or a pharmaceutically acceptable salt thereof, wherein Ring E is a pyrimidine, pyrimidinone, pyridazine, or pyridazinone ring.

10. The compound of claim 3, or a pharmaceutically acceptable salt thereof, wherein Ring E is a pyrimidine ring.

11. The compound of claim 3, or a pharmaceutically acceptable salt thereof, wherein q is 0, 1, or 2.

12. The compound of claim 2, or a pharmaceutically acceptable salt thereof, wherein each R^{4A} is independently selected from C₁₋₆ aliphatic and —OC₁₋₆ aliphatic.

13. The compound of claim 2, or a pharmaceutically acceptable salt thereof, wherein each R^{4A} is independently selected from methyl and methoxy.

14. The compound of claim 1, or a pharmaceutically acceptable salt thereof, wherein R⁶ is hydrogen, deuterium, or optionally substituted C₁₋₆ aliphatic.

15. The compound of claim 1, or a pharmaceutically acceptable salt thereof, wherein R⁶ is hydrogen or deuterium.

16. The compound of claim 1, or a pharmaceutically acceptable salt thereof, wherein R⁷ is hydrogen, deuterium, or optionally substituted C₁₋₆ aliphatic.

17. The compound of claim 1, or a pharmaceutically acceptable salt thereof, wherein R⁷ is hydrogen or deuterium.

18. The compound of claim 1, or a pharmaceutically acceptable salt thereof, wherein R⁶ and R⁷ are each hydrogen.

19. The compound of claim 1, or a pharmaceutically acceptable salt thereof, wherein R⁶ and R⁷ are each deuterium.

20. The compound of claim 1, or a pharmaceutically acceptable salt thereof, wherein D² is C—L^{D2}-R⁹.

21. The compound of claim 1, or a pharmaceutically acceptable salt thereof, wherein L^{D2} is a covalent bond.

22. The compound of claim 1, or a pharmaceutically acceptable salt thereof, wherein R⁹ is hydrogen.

23. The compound of claim 1, or a pharmaceutically acceptable salt thereof, wherein D² is CH.

24. The compound of claim 1, or a pharmaceutically acceptable salt thereof, wherein D² is N.

25. The compound of claim 1, or a pharmaceutically acceptable salt thereof, wherein D³ is CR¹⁰.

26. The compound of claim 1, or a pharmaceutically acceptable salt thereof, wherein R¹⁰ is hydrogen or halogen.

27. The compound of claim 1, or a pharmaceutically acceptable salt thereof, wherein R¹⁰ is fluoro.

28. The compound of claim 1, or a pharmaceutically acceptable salt thereof, wherein D³ is CF.

29. The compound of claim 1, or a pharmaceutically acceptable salt thereof, wherein Ring B is 3- to 7-membered saturated or partially unsaturated monocyclic heterocyclylene having 1-3 heteroatoms independently selected from nitrogen, oxygen, and sulfur.

30. The compound of claim 1, or a pharmaceutically acceptable salt thereof, wherein Ring B is 6-membered saturated or partially unsaturated monocyclic heterocyclylene having 1-3 nitrogen atoms.

31. The compound of claim 1, or a pharmaceutically acceptable salt thereof, wherein n is 0 or 2.

32. The compound of claim 1, or a pharmaceutically acceptable salt thereof, wherein n is 0.

33. The compound of claim 1, or a pharmaceutically acceptable salt thereof, wherein

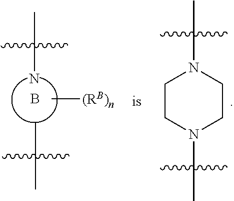

34. The compound of claim 1, or a pharmaceutically acceptable salt thereof, wherein Ring C is 5- to 6-membered monocyclic heteroaryl having 1-4 heteroatoms independently selected from nitrogen, oxygen, and sulfur.

35. The compound of claim 1, or a pharmaceutically acceptable salt thereof, wherein Ring C is 6-membered monocyclic heteroaryl having 1-2 nitrogen atoms.

36. The compound of claim 1, or a pharmaceutically acceptable salt thereof, wherein Ring C is phenyl.

37. The compound of claim 1, or a pharmaceutically acceptable salt thereof, wherein Ring C is

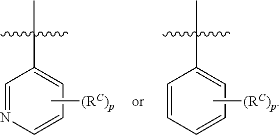

38. The compound of claim 1, or a pharmaceutically acceptable salt thereof, wherein p is 1, 2, or 3.

39. The compound of claim 1, or a pharmaceutically acceptable salt thereof, wherein p is 2.

40. The compound of claim 1, or a pharmaceutically acceptable salt thereof, wherein p is 3.

41. The compound of claim 1, or a pharmaceutically acceptable salt thereof, wherein 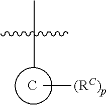
is selected from

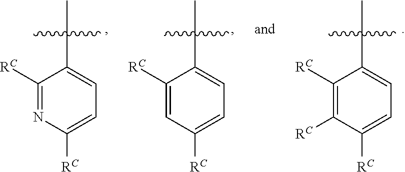

42. The compound of claim 1, or a pharmaceutically acceptable salt thereof, wherein each L^{RC} is a covalent bond.

43. The compound of claim 1, or a pharmaceutically acceptable salt thereof, wherein each R¹² is independently selected from halogen, C₁₋₆ aliphatic, —C(O)N(R)₂, and —C(O)NR(OR).

44. The compound of claim 1, or a pharmaceutically acceptable salt thereof, wherein each R¹² is independently selected from fluoro, methyl, —C(O)NHR, and —C(O)NH(OR).

45. The compound of claim 1, or a pharmaceutically acceptable salt thereof, wherein each R is independently selected from hydrogen, C₁₋₆ aliphatic, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.

46. The compound of claim 1, or a pharmaceutically acceptable salt thereof, wherein 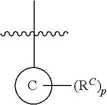
is selected from 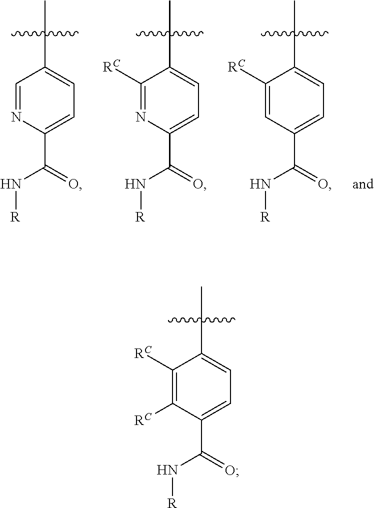
each R^{C} is independently selected from methyl and fluoro; and
each R is independently selected from hydrogen, C₁₋₆ aliphatic, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur.

47. The compound of claim 1, or a pharmaceutically acceptable salt thereof, wherein each R is independently selected from methyl, cyclopropyl, methoxy, cyclopropylmethoxy, cyanocyclopropyl, cyanomethylcyclopropyl, hydroxymethylcyclopropyl, methoxymethylcyclopropyl, cyclobutyl, cyanocyclobutyl, hydroxycyclobutyl, difluorocyclobutyl, cyanocyclohexyl, tetrahydropyranyl, tetrahydrofuranyl, 3-oxabicyclo[3.1.0]hexanyl, methylpyrrolidinyl, and methylpiperidinyl.

48. The compound of claim 1, or a pharmaceutically acceptable salt thereof, wherein,

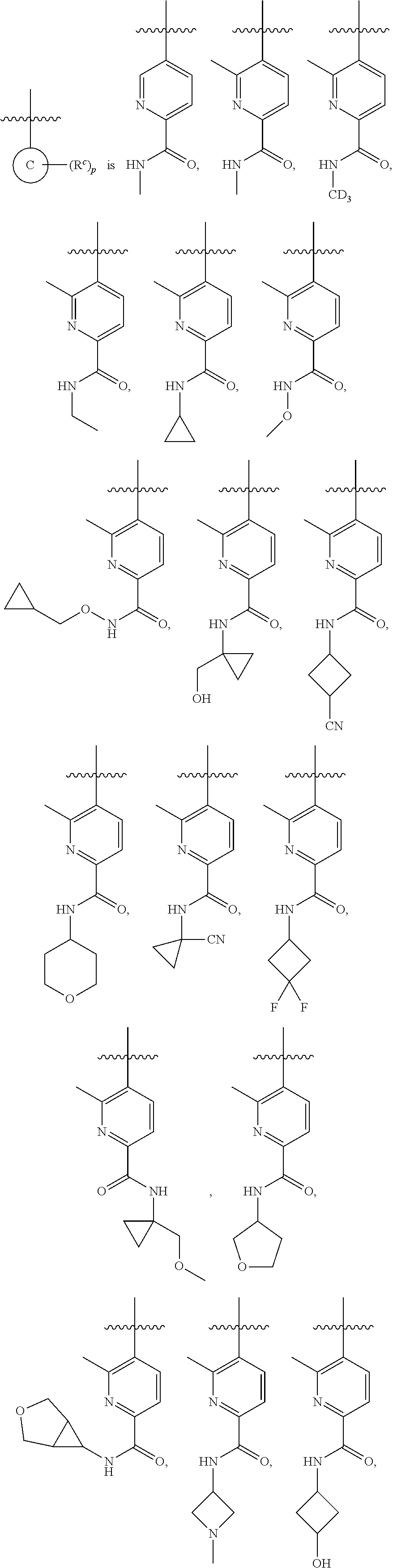

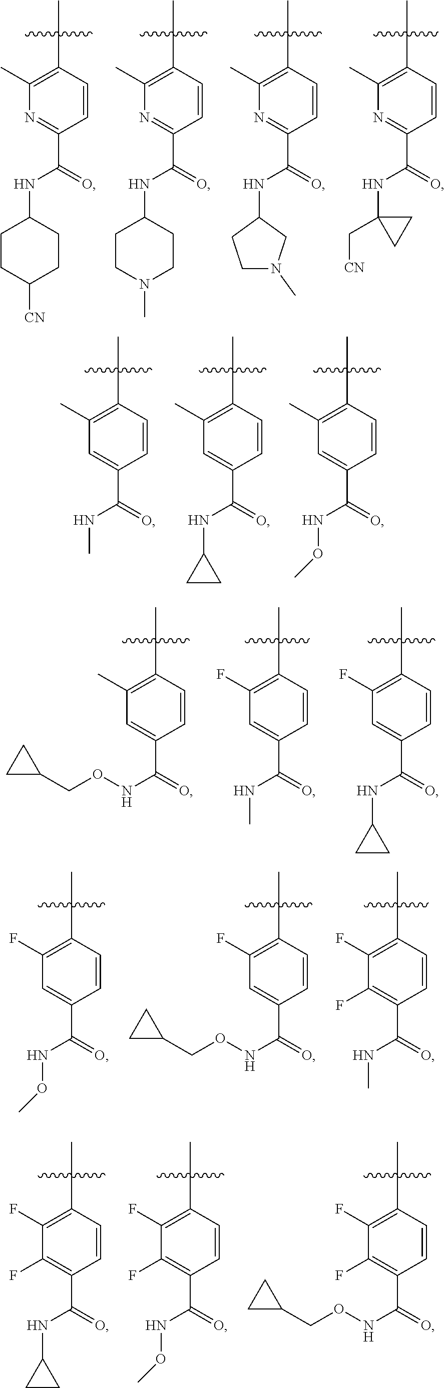

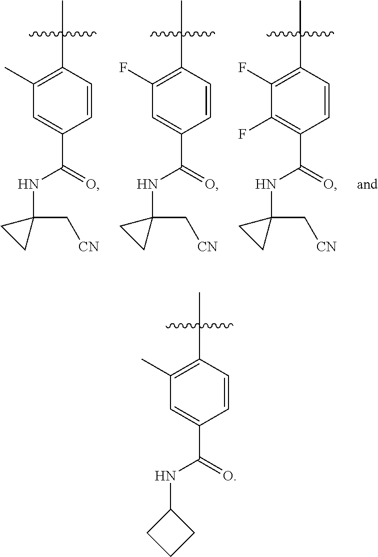

49. The compound of claim 3, or a pharmaceutically acceptable salt thereof, wherein:
X is —N(R^{a})—;
R^{a} is —L^{R3}-R³;
L^{R3} is a covalent bond;
R³ is hydrogen or optionally substituted C₁₋₆ aliphatic;
Ring E is selected from phenyl and 5- to 6-membered monocyclic heteroaryl having 1-4 heteroatoms independently selected from nitrogen, oxygen, and sulfur;
each R^{4A} is independently selected from C₁₋₆ aliphatic and —OC₁₋₆ aliphatic;
R⁶ is hydrogen, deuterium, or optionally substituted C₁₋₆ aliphatic;
R⁷ is hydrogen, deuterium, or optionally substituted C₁₋₆ aliphatic;
D² is CH;
D³ is CR¹⁰;
R¹⁰ is hydrogen or halogen;
Ring B is 3- to 7-membered saturated or partially unsaturated monocyclic heterocyclylene having 1-3 heteroatoms independently selected from nitrogen, oxygen, and sulfur;
Ring C is 5- to 6-membered monocyclic heteroaryl having 1-4 heteroatoms independently selected from nitrogen, oxygen, and sulfur;
each L^{RC} is a covalent bond;
each R¹² is independently selected from halogen, C₁₋₆ aliphatic, —C(O)N(R)₂, and —C(O)NR(OR);
each R is independently selected from hydrogen, C₁₋₆ aliphatic, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur;
n is 0 or 2;
p is 1, 2, or 3; and
q is 0, 1, or 2.

50. The compound of claim 3, or a pharmaceutically acceptable salt thereof, wherein:
X is —N(R^{a})—;
R^{a} is —L^{R3}-R³;
L^{R3} is a covalent bond;
R³ is hydrogen or optionally substituted C₁₋₆ aliphatic;
Ring E is a pyrimidine, pyrimidinone, pyridazine, or pyridazinone ring;
each R^{4A} is independently selected from C₁₋₆ aliphatic and —OC₁₋₆ aliphatic;
R⁶ is hydrogen, deuterium, or optionally substituted C₁₋₆ aliphatic;
R⁷ is hydrogen, deuterium, or optionally substituted C₁₋₆ aliphatic;
D² is CH;
D³ is CR¹⁰;
R¹⁰ is hydrogen or halogen;
Ring B is 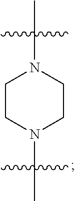 [SMILES: *N1CCN(*)CC1]
Ring C is phenyl or 5- to 6-membered monocyclic heteroaryl having 1-4 heteroatoms independently selected from nitrogen, oxygen, and sulfur;
each L^{RC} is a covalent bond;
each R¹² is independently selected from halogen, C₁₋₆ aliphatic, —C(O)N(R)₂, and —C(O)NR(OR);
each R is independently selected from hydrogen, C₁₋₆ aliphatic, 3- to 7-membered saturated or partially unsaturated monocyclic carbocyclyl, 3- to 7-membered saturated or partially unsaturated monocyclic heterocyclyl having 1-3 heteroatoms independently selected from nitrogen, oxygen, and sulfur, and 6- to 10-membered saturated or partially unsaturated bicyclic heterocyclyl having 1-4 heteroatoms independently selected from nitrogen, oxygen, and sulfur;
n is 0;
p is 1, 2, or 3; and
q is 0, 1, or 2.

51. The compound of claim 1, wherein the compound is of Formula IX-a: 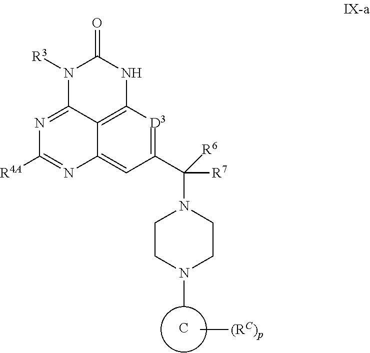
or a pharmaceutically acceptable salt thereof.

52. The compound of claim 1- or 2, wherein the compound is selected from:
5-(4-((3-ethyl-2,4-dioxo-1,2,3,4-tetrahydroquinazolin-7-yl)methyl)piperazin-1-yl)-N-methylpicolinamide;
5-(4-((3-ethyl-2-oxo-1,2,3,4-tetrahydroquinazolin-7-yl)methyl)piperazin-1-yl)-N-methylpicolinamide;
N-methyl-5-(4-((2-oxo-1a,2,3,7b-tetrahydro-1H-cyclopropa[c]quinolin-5-yl)methyl)piperazin-1-yl)picolinamide;
N-methyl-5-(4-((4-oxo-2,3,4,5-tetrahydro-1H-cyclopenta[c]quinolin-7-yl)methyl)piperazin-1-yl)picolinamide;
N-methyl-5-(4-((2′-oxo-1′,4′-dihydro-2′H-spiro[cyclopropane-1,3′-quinolin]-7′-yl)methyl)piperazin-1-yl)picolinamide;
N-methyl-5-(4-((6-oxo-6,7,8,9-tetrahydro-5H-cyclopenta[c][1,5]naphthyridin-3-yl)methyl)piperazin-1-yl)picolinamide;
N-methyl-5-(4-((3-methyl-4-oxo-4,5-dihydro-3H-pyrrolo[2,3-c]quinolin-7-yl)methyl)piperazin-1-yl)picolinamide;
N-methyl-5-(4-((1-methyl-4-oxo-4,5-dihydro-1H-pyrrolo[3,2-c]quinolin-7-yl)methyl)piperazin-1-yl)picolinamide;
5-(4-((3-ethyl-2,4-dioxo-1,2,3,4-tetrahydropyrido[3,2-d]pyrimidin-7-yl)methyl)piperazin-1-yl)-N-methylpicolinamide;
N-methyl-5-(4-((3-methyl-4-oxo-4,5-dihydro-3H-pyrazolo[3,4-c]quinolin-7-yl)methyl)piperazin-1-yl)picolinamide;
5-(4-((3-ethyl-2-oxo-1,2,3,4-tetrahydropyrido[3,2-d]pyrimidin-7-yl)methyl)piperazin-1-yl)-N-methylpicolinamide;
5-(4-((3-(2,2-difluoroethyl)-2,4-dioxo-1,2,3,4-tetrahydroquinazolin-7-yl)methyl)piperazin-1-yl)-N-methylpicolinamide;
5-(4-((6-ethyl-5-oxo-4,5-dihydrothieno[3,2-b]pyridin-2-yl)methyl)piperazin-1-yl)-N-methylpicolinamide;
5-(4-((4-ethyl-5-oxo-2,3,5,6-tetrahydropyrano[4,3,2-de]quinolin-8-yl)methyl)piperazin-1-yl)-N-methylpicolinamide;
5-(4-((3-ethyl-2-oxo-2,3-dihydro-1H-pyrimido[4,5,6-de]quinazolin-8-yl)methyl)piperazin-1-yl)-N-methylpicolinamide;
5-(4-((3-ethyl-2,4-dioxo-1,2,3,4-tetrahydroquinazolin-7-yl)methyl)piperazin-1-yl)-N,6-dimethylpicolinamide;
5-(4-((3-ethyl-2-oxo-2,3-dihydro-1H-pyrimido[4,5,6-de]quinazolin-8-yl)methyl)piperazin-1-yl)-N,6-dimethylpicolinamide;
5-(4-((3-ethyl-2-oxo-2,3-dihydro-1H-pyrimido[4,5,6-de]quinazolin-8-yl)methyl)piperazin-1-yl)-N-methyl-6-(trifluoromethyl)picolinamide;
5-(4-((3-ethyl-2-oxo-2,3-dihydro-1H-pyrimido[4,5,6-de]quinazolin-8-yl)methyl)piperazin-1-yl)-6-fluoro-N-methylpicolinamide;
N,6-dimethyl-5-(4-((6-oxo-6,7,8,9-tetrahydro-5H-cyclopenta[c][1,5]naphthyridin-3-yl)methyl)piperazin-1-yl)picolinamide;
5-(4-((3-ethyl-8-fluoro-2,4-dioxo-1,2,3,4-tetrahydroquinazolin-7-yl)methyl)piperazin-1-yl)-N,6-dimethylpicolinamide;
5-(4-((3-ethyl-5-fluoro-2,4-dioxo-1,2,3,4-tetrahydroquinazolin-7-yl)methyl)piperazin-1-yl)-N,6-dimethylpicolinamide;
N-methyl-5-(4-((6-oxo-6,7,8,9-tetrahydro-5H-cyclopenta[c][1,5]naphthyridin-3-yl)methyl)piperazin-1-yl)-6-(trifluoromethyl)picolinamide;
6-fluoro-N-methyl-5-(4-((6-oxo-6,7,8,9-tetrahydro-5H-cyclopenta[c][1,5]naphthyridin-3-yl)methyl)piperazin-1-yl)picolinamide;
5-(4-((4-fluoro-6-oxo-6,7,8,9-tetrahydro-5H-cyclopenta[c][1,6]naphthyridin-3-yl)methyl)piperazin-1-yl)-N,6-dimethylpicolinamide;
5-(4-((4-fluoro-6-oxo-6,7,8,9-tetrahydro-5H-cyclopenta[c][1,6]naphthyridin-3-yl)methyl)piperazin-1-yl)-N-methylpicolinamide;
6-fluoro-5-(4-((4-fluoro-6-oxo-6,7,8,9-tetrahydro-5H-cyclopenta[c][1,6]naphthyridin-3-yl)methyl)piperazin-1-yl)-N-methylpicolinamide;
5-(4-((4-fluoro-6-oxo-6,7,8,9-tetrahydro-5H-cyclopenta[c][1,6]naphthyridin-3-yl)methyl)piperazin-1-yl)-N-methyl-6-(trifluoromethyl)picolinamide;
5-(4-((3-ethyl-2,4-dioxo-1,2,3,4-tetrahydroquinazolin-7-yl)methyl)piperazin-1-yl)-6-fluoro-N-methylpicolinamide;
N,6-dimethyl-5-(4-((6-oxo-6,7,8,9-tetrahydro-5H-cyclopenta[c][1,6]naphthyridin-3-yl)methyl)piperazin-1-yl)picolinamide;
5-(4-((3-ethyl-2,4-dioxo-1,2,3,4-tetrahydroquinazolin-7-yl)methyl)piperazin-1-yl)-N-methyl-6-(trifluoromethyl)picolinamide;
5-(4-((5-chloro-3-ethyl-2,4-dioxo-1,2,3,4-tetrahydroquinazolin-7-yl)methyl)piperazin-1-yl)-N,6-dimethylpicolinamide; and
5-(4-((3-ethyl-5-fluoro-2,4-dioxo-1,2,3,4-tetrahydroquinazolin-7-yl)methyl)piperazin-1-yl)-6-fluoro-N-methylpicolinamide;
or a pharmaceutically acceptable salt thereof.

53. The compound of claim 1- or 2, wherein the compound is selected from:
5-(4-((3-ethyl-6-fluoro-1-methyl-4-oxo-1,3,4,5-tetrahydropyrazolo[3,4,5-de]quinazolin-7-yl)methyl)piperazin-1-yl)-N,6-dimethylpicolinamide;
5-(4-((3-ethyl-9-fluoro-2-oxo-2,3-dihydro-1H-pyrimido[4,5,6-de]quinazolin-8-yl)methyl)piperazin-1-yl)-N,6-dimethylpicolinamide;
5-(4-((3-ethyl-5-fluoro-2,4-dioxo-1,2,3,4-tetrahydroquinazolin-7-yl)methyl)piperazin-1-yl)-N,6-bis(methyl-d3)picolinamide;
5-(4-((5-(difluoromethyl)-3-ethyl-2,4-dioxo-1,2,3,4-tetrahydroquinazolin-7-yl)methyl)piperazin-1-yl)-N,6-dimethylpicolinamide;
5-(4-((8-fluoro-5-methoxy-3-methyl-2,4-dioxo-1,2,3,4-tetrahydroquinazolin-7-yl)methyl)piperazin-1-yl)-N,6-dimethylpicolinamide;
5-(4-((5-chloro-3-ethyl-8-fluoro-2,4-dioxo-1,2,3,4-tetrahydroquinazolin-7-yl)methyl)piperazin-1-yl)-N,6-dimethylpicolinamide;
5-(4-((5-cyclopropyl-3-ethyl-8-fluoro-2,4-dioxo-1,2,3,4-tetrahydroquinazolin-7-yl)methyl)piperazin-1-yl)-N,6-dimethylpicolinamide;
5-(4-((3-ethyl-8-fluoro-5-(hydroxymethyl)-2,4-dioxo-1,2,3,4-tetrahydroquinazolin-7-yl)methyl)piperazin-1-yl)-N,6-dimethylpicolinamide;
5-(4-((5-(cyanomethyl)-3-ethyl-8-fluoro-2,4-dioxo-1,2,3,4-tetrahydroquinazolin-7-yl)methyl)piperazin-1-yl)-N,6-dimethylpicolinamide;
5-(4-((5-chloro-3-methyl-2,4-dioxo-1,2,3,4-tetrahydroquinazolin-7-yl)methyl)piperazin-1-yl)-N-ethyl-6-methylpicolinamide;
5-(4-((3-ethyl-2,4-dioxo-1,2,3,4-tetrahydroquinazolin-7-yl)methyl)piperazin-1-yl)-N,6-bis(methyl-d3)picolinamide;
5-[4-[(12-ethyl-11-oxo-2,3,10,12-tetrazatricyclo[7.3.1.05,13]trideca-1,3,5,7,9 (13)-pentaen-7-yl)methyl]piperazin-1-yl]-N,6-dimethyl-pyridine-2-carboxamid;
5-(4-((3-ethyl-8-fluoro-5-(methoxymethyl)-2,4-dioxo-1,2,3,4-tetrahydroquinazolin-7-yl)methyl)piperazin-1-yl)-N,6-dimethylpicolinamide;
5-(4-((5-(difluoromethyl)-3-methyl-2,4-dioxo-1,2,3,4-tetrahydroquinazolin-7-yl)methyl)piperazin-1-yl)-N,6-dimethylpicolinamide;
5-(4-((5-chloro-3-ethyl-2,4-dioxo-1,2,3,4-tetrahydroquinazolin-7-yl)methyl)piperazin-1-yl)-N-ethyl-6-methylpicolinamide; and
5-(4-((5-cyclopropyl-8-fluoro-3-methyl-2,4-dioxo-1,2,3,4-tetrahydroquinazolin-7-yl)methyl)piperazin-1-yl)-N,6-dimethylpicolinamide;
or a pharmaceutically acceptable salt thereof.

54. The compound of claim 1, wherein the compound is selected from:
5-(4-((3-ethyl-9-fluoro-5-methoxy-2-oxo-2,3-dihydro-1H-pyrimido[4,5,6-de]quinazolin-8-yl)methyl)piperazin-1-yl)-N,6-dimethylpicolinamide;
5-(4-((3-ethyl-9-fluoro-6-methyl-2,5-dioxo-2,3,5,6-tetrahydro-1H-pyrimido[4,5,6-de]quinazolin-8-yl)methyl)piperazin-1-yl)-N,6-dimethylpicolinamide;
5-(4-((3-ethyl-9-fluoro-5-methyl-2-oxo-2,3-dihydro-1H-pyrimido[4,5,6-de]quinazolin-8-yl)methyl)piperazin-1-yl)-N,6-dimethylpicolinamide;
5-(4-((3-ethyl-9-fluoro-2-oxo-2,3-dihydro-1H-pyrimido[4,5,6-de]quinazolin-8-yl)methyl-d2)piperazin-1-yl)-N,6-dimethylpicolinamide;
5-(4-((9-ethyl-6-fluoro-3-methyl-8-oxo-8,9-dihydro-7H-pyridazino[3,4,5-de]quinazolin-5-yl)methyl)piperazin-1-yl)-N,6-dimethylpicolinamide;
N-cyclopropyl-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-cyclopropyl-5-(4-((9-ethyl-6-fluoro-3-methyl-8-oxo-8,9-dihydro-7H-pyridazino[3,4,5-de]quinazolin-5-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-N-methoxy-6-methylpicolinamide;
N-(cyclopropylmethoxy)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-N-(1-(hydroxymethyl)cyclopropyl)-6-methylpicolinamide;
N-((1r,3r)-3-cyanocyclobutyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydro-2H-pyran-4-yl)picolinamide;
N-(1-cyanocyclopropyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
(S)-5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydro-2H-pyran-3-yl)picolinamide;
N-(3,3-difluorocyclobutyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-((1s,3s)-3-cyanocyclobutyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-N-(1-(methoxymethyl)cyclopropyl)-6-methylpicolinamide;
(R)-5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydrofuran-3-yl)picolinamide;
(R)-5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydro-2H-pyran-3-yl)picolinamide;
N-((1R,5S,6s)-3-oxabicyclo[3.1.0]hexan-6-yl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-((1R,5S,6r)-3-oxabicyclo[3.1.0]hexan-6-yl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-6-methyl-N-(1-methylazetidin-3-yl)picolinamide;
5-(4-((3-ethyl-9-fluoro-2-oxo-2,3-dihydro-1H-pyrimido[4,5,6-de]quinazolin-8-yl)methyl)piperazin-1-yl)-N-((1s,3s)-3-hydroxycyclobutyl)-6-methylpicolinamide;
(S)-5-(4-((3-ethyl-9-fluoro-2-oxo-2,3-dihydro-1H-pyrimido[4,5,6-de]quinazolin-8-yl)methyl)piperazin-1-yl)-6-methyl-N-(tetrahydrofuran-3-yl)picolinamide;
N-((1s,4s)-4-cyanocyclohexyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-((1r,4r)-4-cyanocyclohexyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
5-(4-((3-ethyl-9-fluoro-2-oxo-2,3-dihydro-1H-pyrimido[4,5,6-de]quinazolin-8-yl)methyl)piperazin-1-yl)-6-methyl-N-(1-methylpiperidin-4-yl)picolinamide;
(R)-5-(4-((3-ethyl-9-fluoro-2-oxo-2,3-dihydro-1H-pyrimido[4,5,6-de]quinazolin-8-yl)methyl)piperazin-1-yl)-6-methyl-N-(1-methylpyrrolidin-3-yl)picolinamide;
(S)-5-(4-((3-ethyl-9-fluoro-2-oxo-2,3-dihydro-1H-pyrimido[4,5,6-de]quinazolin-8-yl)methyl)piperazin-1-yl)-6-methyl-N-(1-methylpyrrolidin-3-yl)picolinamide;
N-(1-(cyanomethyl)cyclopropyl)-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
N-cyclopropyl-5-(4-((9-ethyl-6-fluoro-2-methyl-3,8-dioxo-2,7,8,9-tetrahydro-3H-pyridazino[3,4,5-de]quinazolin-5-yl)methyl)piperazin-1-yl)-6-methylpicolinamide;
4-[4-[(6-ethyl-10-fluoro-7-oxo-2,4,6,8-tetrazatricyclo[7.3.1.05,13]trideca-1,3,5 (13),9,11-pentaen-11-yl)methyl]piperazin-1-yl]-N,3-dimethyl-benzamide;
N-cyclopropyl-4-(4-((3-ethyl-9-fluoro-2-oxo-2,3-dihydro-1H-pyrimido[4,5,6-de]quinazolin-8-yl)methyl)piperazin-1-yl)-3-methylbenzamide;
4-(4-((3-ethyl-9-fluoro-2-oxo-2,3-dihydro-1H-pyrimido[4,5,6-de]quinazolin-8-yl)methyl)piperazin-1-yl)-N-methoxy-3-methylbenzamide;
N-(cyclopropylmethoxy)-4-(4-((3-ethyl-9-fluoro-2-oxo-2,3-dihydro-1H-pyrimido[4,5,6-de]quinazolin-8-yl)methyl)piperazin-1-yl)-3-methylbenzamide;
4-(4-((3-ethyl-9-fluoro-2-oxo-2,3-dihydro-1H-pyrimido[4,5,6-de]quinazolin-8-yl)methyl)piperazin-1-yl)-3-fluoro-N-methylbenzamide;
N-cyclopropyl-4-(4-((3-ethyl-9-fluoro-2-oxo-2,3-dihydro-1H-pyrimido[4,5,6-de]quinazolin-8-yl)methyl)piperazin-1-yl)-3-fluorobenzamide;
4-(4-((3-ethyl-9-fluoro-2-oxo-2,3-dihydro-1H-pyrimido[4,5,6-de]quinazolin-8-yl)methyl)piperazin-1-yl)-3-fluoro-N-methoxybenzamide;
N-(cyclopropylmethoxy)-4-(4-((3-ethyl-9-fluoro-2-oxo-2,3-dihydro-1H-pyrimido[4,5,6-de]quinazolin-8-yl)methyl)piperazin-1-yl)-3-fluorobenzamide;
4-(4-((3-ethyl-9-fluoro-2-oxo-2,3-dihydro-1H-pyrimido[4,5,6-de]quinazolin-8-yl)methyl)piperazin-1-yl)-2,3-difluoro-N-methylbenzamide;
N-cyclopropyl-4-(4-((3-ethyl-9-fluoro-2-oxo-2,3-dihydro-1H-pyrimido[4,5,6-de]quinazolin-8-yl)methyl)piperazin-1-yl)-2,3-difluorobenzamide;
4-(4-((3-ethyl-9-fluoro-2-oxo-2,3-dihydro-1H-pyrimido[4,5,6-de]quinazolin-8-yl)methyl)piperazin-1-yl)-2,3-difluoro-N-methoxybenzamide;
N-(cyclopropylmethoxy)-4-(4-((3-ethyl-9-fluoro-2-oxo-2,3-dihydro-1H-pyrimido[4,5,6-de]quinazolin-8-yl)methyl)piperazin-1-yl)-2,3-difluorobenzamide;
N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-3-methylbenzamide;
N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-3-fluorobenzamide;
N-(1-(cyanomethyl)cyclopropyl)-4-(4-((3-ethyl-9-fluoro-2-oxo-2,3-dihydro-1H-pyrimido[4,5,6-de]quinazolin-8-yl)methyl)piperazin-1-yl)-2,3-difluorobenzamide; and
N-cyclobutyl-5-(4-((3-ethyl-9-fluoro-2-oxo-2,3-dihydro-1H-pyrimido[4,5,6-de]quinazolin-8-yl)methyl)piperazin-1-yl)-6-methylpicolinamide;
or a pharmaceutically acceptable salt thereof.

55. A pharmaceutical composition comprising a compound of claim 1, or a pharmaceutically acceptable salt thereof, and a pharmaceutically acceptable carrier.

56. A method of inhibiting PARP1, comprising administering to a subject the compound of claim 1, or a pharmaceutically acceptable salt thereof.

57. A method of treating a disease, disorder, or condition associated with PARP1, comprising administering to a subject in need thereof the compound of claim 1, or a pharmaceutically acceptable salt thereof.

58. A method of treating cancer, comprising administering to a subject in need thereof the compound of claim 1, or a pharmaceutically acceptable salt thereof.
